(19) United States
(12) Patent Application Publication
(10) Pub. No.: US 2023/0257387 A1
(43) Pub. Date: Aug. 17, 2023

(54) ORGANIC COMPOUND, ELECTRONIC ELEMENT AND ELECTRONIC DEVICE THEREOF

(71) Applicant: SHAANXI LIGHTE OPTOELECTRONICS MATERIAL CO., LTD., Xi’an (CN)

(72) Inventors: Na YUE, Xi'an (CN); Zhengshen HUA, Xi'an (CN); Youngkook KIM, Xi'an (CN); Yingwen LI, Xi'an (CN)

(21) Appl. No.: 18/012,913

(22) PCT Filed: Mar. 18, 2022
(86) PCT No.: PCT/CN2022/081751

§ 371 Date: Dec. 23, 2022

(30) Foreign Application Priority Data
Apr. 8, 2021 (CN) 202110377495.X

(51) Int. Cl. C07D 487/06; H10K 85/60

(52) U.S. Cl. CPC C07D 487/06; H10K 85/6572; H10K 85/636; H10K 85/6574; H10K 85/631; H10K 85/633; H10K 85/6576; H10K 85/626; C07B 2200/05

(57) ABSTRACT

The present disclosure provides an organic compound and an electronic element and electronic device thereof, and belongs to the technical field of organic electroluminescence. The organic compound of the present disclosure includes a fused conjugated ring system and stereo-arylamino, which can effectively improve the thermal stability, membrane stability, and carrier mobility of the material. When used for an organic light-emitting device (OLED), the organic compound of the present disclosure can improve the light-emitting efficiency and life span of the OLED.

### CROSS REFERENCE TO RELATED APPLICATION

The present application claims priority of Chinese Patent Application 202110377495.X filed to the China National Intellectual Property Administration (CNIPA) on Apr. 8, 2021, which is incorporated herein by reference in its entirety.

### TECHNICAL FIELD

The present application relates to the technical field of organic electroluminescent materials, and in particular to an organic compound, and an electronic element and electronic device thereof.

### BACKGROUND

Currently, organic light-emitting elements (OLEDs) have been widely used in mobile phones, computers, lighting, and other fields due to their advantages such as high luminance, fast response, and wide adaptability. Organic electroluminescent elements (OLEDs) are thin-film elements manufactured from organic light-emitting materials, and can emit light under the excitation of an electric field. In addition to an electrode material film layer, an organic electroluminescent element needs to have different organic functional material layers. π-bond or anti-π-bond orbitals of organic functional materials lead to shifted valences and conductivity, and the overlap of the orbitals leads to a highest occupied molecular orbital (HOMO) and a lowest unoccupied molecular orbital (LUMO), which achieves charge transfer through intermolecular transition.

In a structure of an OLED, an electron blocking layer (EBL) is arranged to block electrons transmitted from an organic light-emitting layer, thereby ensuring that an electron and a hole can be efficiently recombined in the organic light-emitting layer; the EBL can also block excitons diffused from the organic light-emitting layer to reduce the triplet-state quenching of the excitons, thereby ensuring the light-emitting efficiency of the OLED; and a compound of the EBL has a relatively-high LUMO value, which can effectively block the transmission and diffusion of electrons and excitons from the organic light-emitting layer to an anode.

The continuous improvement of performance of OLEDs requires not only the innovation in the structure and manufacturing process for OLEDs, but also the continuous research and innovation of organic electroluminescent materials. At present, in order to improve the performance of an OLED by changing organic functional materials, it is necessary to reduce a driving voltage of the element and improve the light-emitting efficiency and service life of the element.

### SUMMARY

The present disclosure is intended to overcome the above-mentioned deficiencies in the prior art, and to provide an organic compound, and an electronic element and electronic device thereof.

According to a first aspect of the present disclosure, an organic compound is provided, which has a general structure shown in chemical formula 1:

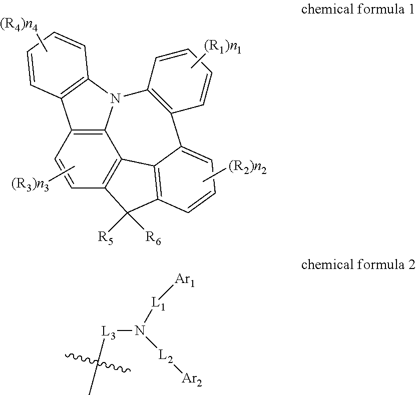

wherein R₅ and R₆ are the same or different, and are each independently selected from the group consisting of alkyl with 1 to 10 carbon atoms, aryl with 6 to 20 carbon atoms, haloalkyl with 1 to 10 carbon atoms, cycloalkyl with 3 to 10 carbon atoms, heteroaryl with 3 to 20 carbon atoms, hydrogen, deuterium, halogen, and cyano; or R₅ and R₆ are optionally connected to each other to form a substituted or unsubstituted 5- to 18-membered aliphatic ring or 5- to 18-membered aromatic ring together with the carbon atom to which they are jointly connected, and a substituent in the 5- to 18-membered aliphatic ring or 5- to 18-membered aromatic ring is independently selected from the group consisting of deuterium, halogen, cyano, alkyl with 1 to 10 carbon atoms, haloalkyl with 1 to 10 carbon atoms, and deuterated alkyl with 1 to 10 carbon atoms;

R₁, R₂, R₃, and R₄ are the same or different, and are each independently selected from the group consisting of hydrogen, deuterium, halogen, cyano, alkyl with 1 to 10 carbon atoms, cycloalkyl with 3 to 10 carbon atoms, aryl with 6 to 20 carbon atoms, heteroaryl with 3 to 12 carbon atoms, and a group shown in chemical formula 2; and one, two, three, or four of R₁, R₂, R₃, and R₄ are the group shown in chemical formula 2;

R₁, R₂, R₃, and R₄ are collectively represented by Rᵢ, and n₁ to n₄ are collectively represented by nᵢ; nᵢ indicates a number of Rᵢ, and i is a variable of 1, 2, 3, or 4; when i is 1 or 4, nᵢ is selected from the group consisting of 1, 2, 3, and 4; when i is 2, nᵢ is selected from the group consisting of 1, 2, and 3; when i is 3, nᵢ is selected from the group consisting of 1 and 2; and when nᵢ is greater than 1, any two nᵢ values are the same or different;

L₁, L₂, and L₃ are the same or different, and are each independently selected from the group consisting of a single bond, substituted or unsubstituted arylene with 6 to 30 carbon atoms, and substituted or unsubstituted heteroarylene with 3 to 30 carbon atoms;

Ar₁ and Ar₂ are the same or different, and are each independently selected from the group consisting of substituted or unsubstituted aryl with 6 to 30 carbon atoms and substituted or unsubstituted heteroaryl with 3 to 30 carbon atoms;

substituents in L₁, L₂, L₃, Ar₁, and Ar₂ are the same or different, and are each independently selected from the group consisting of deuterium, halogen, cyano, heteroaryl with 3 to 20 carbon atoms, aryl with 6 to 20 carbon atoms, alkyl with 1 to 10 carbon atoms, haloalkyl with 1 to 10 carbon atoms, deuterated alkyl with 1 to 10 carbon atoms, alkoxy with 1 to 10 carbon atoms, cycloalkyl with 3 to 10 carbon atoms, alkylthio with 1 to 10 carbon atoms, trialkylsilyl with 3 to 12 carbon atoms, arylsilyl with 6 to 18 carbon atoms, aryloxy with 6 to 20 carbon atoms, and arylthio with 6 to 20 carbon atoms; or any two adjacent substituents in L₁, L₂, L₃, Ar₁, and Ar₂ are optionally connected to each other to form a 5- to 13-membered aliphatic ring or a 5- to 13-membered aromatic ring.

The organic compound of the present disclosure has a fused-ring parent nucleus of carbazolo-fluorene, and the parent nucleus is a large non-planar conjugated system with high hole mobility, which can effectively improve the steric hindrance of a material to avoid the compound stacking, thereby improving the stability of film formation. In addition, arylamino is linked to the fused ring, which can further effectively reduce the interaction among molecules of the large non-planar conjugated system and reduce the molecule stacking; and a substituent in the arylamino can be adjusted to further improve the hole-transporting ability and reduce an energy gap between a singlet state and a triplet state, such that the organic compound has excellent hole-transporting performance. The organic compound of the present disclosure can be used in a hole transport layer (HTL) or an EBL (also known as hole adjustment layer) of an OLED to reduce the driving voltage of the OLED and improve the light-emitting efficiency and service life of the OLEDs.

According to a second aspect of the present disclosure, an electronic element is provided, including an anode, a cathode, and at least one functional layer between the anode and the cathode, wherein the functional layer includes the organic compound described above.

According to a third aspect of the present disclosure, an electronic device is provided, including the electronic element described above.

It should be understood that the above general description and the following detailed description are merely exemplary and explanatory, and should not be construed as a limitation to the present disclosure.

### BRIEF DESCRIPTION OF THE DRAWINGS

The accompanying drawings incorporated into the specification and constituting a part of the specification illustrate the embodiments of the present disclosure, and are used together with the description to explain the principles of the present disclosure. In these accompanying drawings, similar reference numerals represent similar elements. The accompanying drawings in the following description illustrate some rather than all of the embodiments of the present disclosure. Other accompanying drawings can be derived by persons of ordinary skill in the art based on these accompanying drawings without creative efforts.

FIG. 1 is a schematic structural diagram of an embodiment of the OLED of the present disclosure.

FIG. 2 is a schematic structural diagram of an electronic device in an embodiment of the present disclosure.

### REFERENCE NUMERALS

100 anode; 200 cathode; 300 functional layer; 310 hole injection layer (HIL); 320 HTL; 330 EBL (also known as hole adjustment layer); 340 organic electroluminescent layer; 350 electron transport layer (ETL); 360 electron injection layer (EIL); and 400 electronic device.

### DETAILED DESCRIPTION

Exemplary embodiments will be described below comprehensively with reference to the accompanying drawings. However, the exemplary embodiments can be implemented in various forms and should not be construed as being limited to examples described herein. On the contrary, these embodiments are provided such that the present disclosure is comprehensive and complete, and fully conveys the concept of the exemplary embodiments to those skilled in the art. The described features, structures, or characteristics may be incorporated into one or more embodiments in any suitable manner. In the following description, many specific details are provided to give a full understanding of the embodiments of the present disclosure.

In the figures, a thickness of each of regions and layers may be exaggerated for clarity. The same reference numerals in the figures indicate the same or similar structures, and thus their detailed descriptions will be omitted.

The described features, structures, or characteristics may be incorporated into one or more embodiments in any suitable manner. In the following description, many specific details are provided to give a full understanding of the embodiments of the present disclosure. However, those skilled in the art will be aware that the technical solutions of the present disclosure may be practiced without one or more of the specific details, or other methods, components, materials, and the like may be used. In other cases, well-known structures, materials, or operations are not shown or described in detail to avoid obscuring the main technical ideas of the present disclosure.

The present disclosure provides an organic compound, with a general structure shown in chemical formula 1:

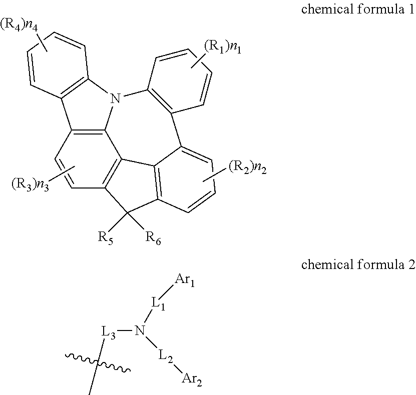

wherein R₅ and R₆ are the same or different, and are each independently selected from the group consisting of alkyl with 1 to 10 carbon atoms, haloalkyl with 1 to 10 carbon atoms, cycloalkyl with 3 to 10 carbon atoms, aryl with 6 to 20 carbon atoms, heteroaryl with 3 to 20 carbon atoms, hydrogen, deuterium, halogen, and cyano; or R₅ and R₆ are optionally connected to each other to form a substituted or unsubstituted 5- to 18-membered aliphatic ring or 5- to 18-membered aromatic ring together with the carbon atom to which they are jointly connected, and a substituent in the 5- to 18-membered aliphatic ring or 5- to 18-membered aromatic ring is independently selected from the group consisting of deuterium, halogen, cyano, alkyl with 1 to 10 carbon atoms, haloalkyl with 1 to 10 carbon atoms, and deuterated alkyl with 1 to 10 carbon atoms;

R₁, R₂, R₃, and R₄ are the same or different, and are each independently selected from the group consisting of hydrogen, deuterium, halogen, cyano, alkyl with 1 to 10 carbon atoms, cycloalkyl with 3 to 10 carbon atoms, aryl with 6 to 20 carbon atoms, heteroaryl with 3 to 12 carbon atoms, and a group shown in chemical formula 2; and one, two, three, or four of R₁, R₂, R₃, and R₄ are the group shown in chemical formula 2;

R₁, R₂, R₃, and R₄ are collectively represented by Rᵢ, and n₁ to n₄ are collectively represented by nᵢ; nᵢ indicates a number of Rᵢ, and i is a variable of 1, 2, 3, or 4; when i is 1 or 4, nᵢ is selected from the group consisting of 1, 2, 3, and 4; when i is 2, nᵢ is selected from the group consisting of 1, 2, and 3; when i is 3, nᵢ is selected from the group consisting of 1 and 2; and when nᵢ is greater than 1, any two nᵢ values are the same or different;

L₁, L₂, and L₃ are the same or different, and are each independently selected from the group consisting of a single bond, substituted or unsubstituted arylene with 6 to 30 carbon atoms, and substituted or unsubstituted heteroarylene with 3 to 30 carbon atoms;

Ar₁ and Ar₂ are the same or different, and are each independently selected from the group consisting of substituted or unsubstituted aryl with 6 to 30 carbon atoms and substituted or unsubstituted heteroaryl with 3 to 30 carbon atoms;

substituents in L₁, L₂, L₃, Ar₁, and Ar₂ are the same or different, and are each independently selected from the group consisting of deuterium, halogen, cyano, heteroaryl with 3 to 20 carbon atoms, aryl with 6 to 20 carbon atoms, alkyl with 1 to 10 carbon atoms, haloalkyl with 1 to 10 carbon atoms, deuterated alkyl with 1 to 10 carbon atoms, alkoxy with 1 to 10 carbon atoms, cycloalkyl with 3 to 10 carbon atoms, alkylthio with 1 to 10 carbon atoms, trialkylsilyl with 3 to 12 carbon atoms, arylsilyl with 6 to 18 carbon atoms, aryloxy with 6 to 20 carbon atoms, and arylthio with 6 to 20 carbon atoms; or any two adjacent substituents in L₁, L₂, L₃, Ar₁, and Ar₂ are optionally connected to each other to form a 5- to 13-membered aliphatic ring or a 5- to 13-membered aromatic ring.

Optionally, only one of the R₁, R₂, R₃, and R₄ is the group shown in chemical formula 2, and the rest may all be hydrogen.

Optionally, the organic compound may have a structure shown in formula 1A, 2A, 3A, or 4A below:

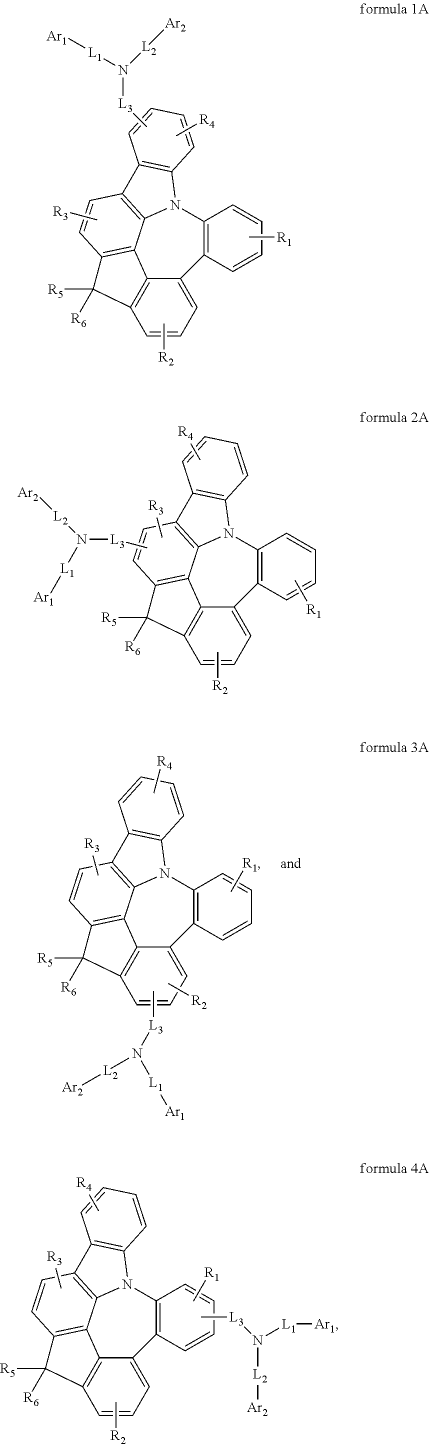

wherein R₁, R₂, R₃, and R₄ each independently selected from the group consisting of hydrogen, deuterium, cyano, fluorine, methyl, ethyl, isopropyl, tert-butyl, trifluoromethyl, trideuteromethyl, trimethylsilyl, phenyl, naphthyl, biphenyl, pyridyl, pyrimidinyl, quinolinyl, isoquinolinyl, carbazolyl, dibenzofuranyl, dibenzothienyl, dimethylfluorenyl, and N-phenylcarbazolyl.

Further optionally, the organic compound may have a structure selected from the group consisting of the following structures:

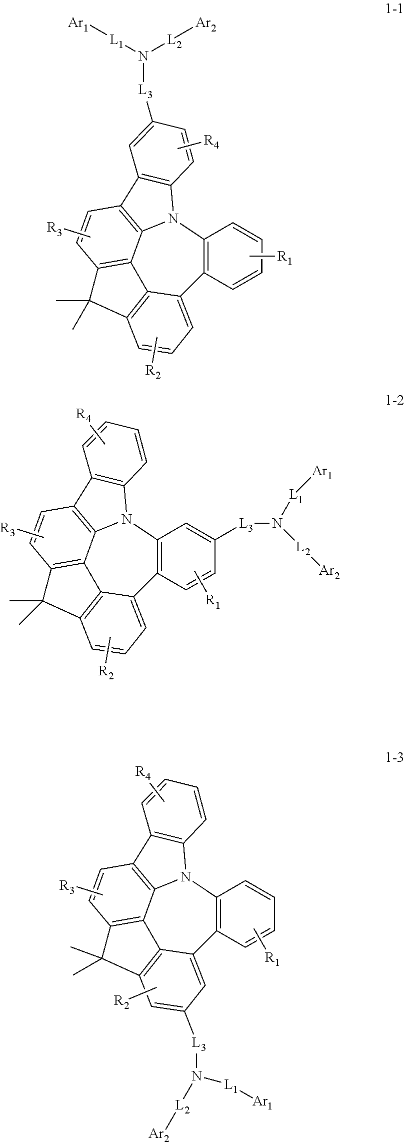

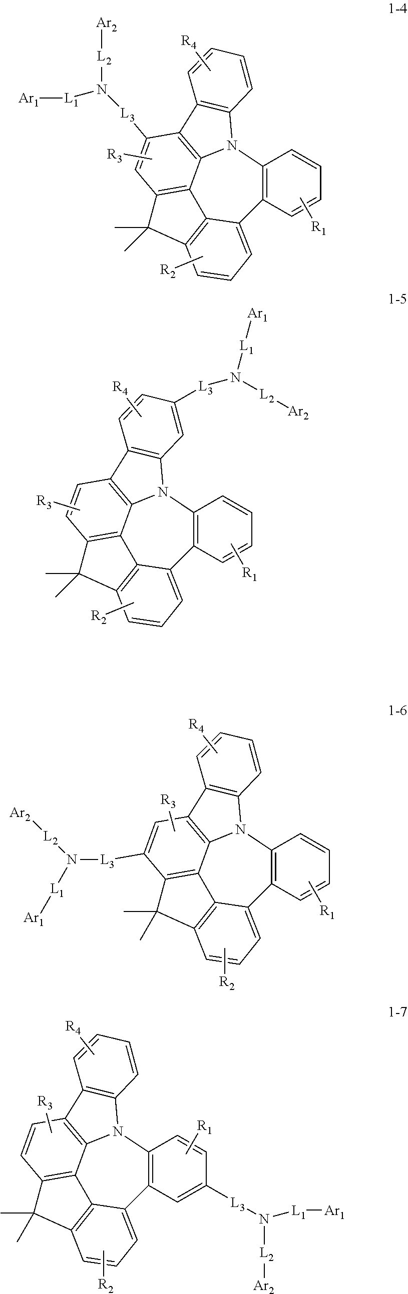

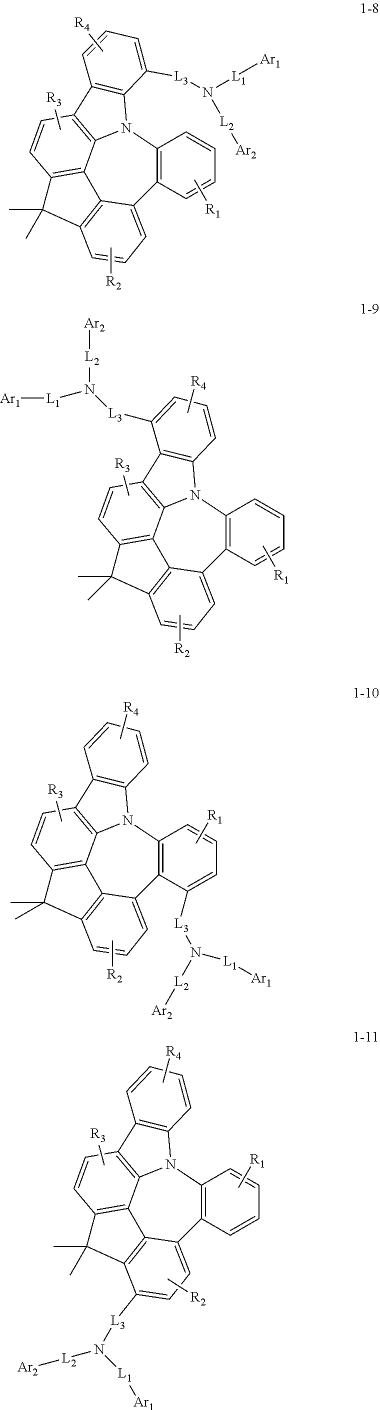

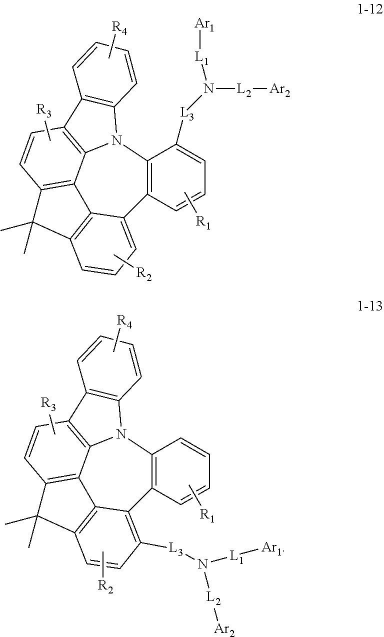

The description manners used in the present disclosure such as “ . . . is (are) each independently selected from the group consisting of” and “each . . . is independently selected from the group consisting of” can be used interchangeably, and should be understood in a broad sense, which can mean that, in different groups, specific options expressed by the same symbol do not affect each other; or in the same group, specific options expressed by the same symbol do not affect each other. For example,“

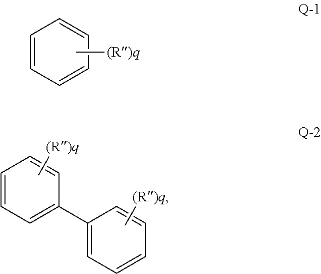

wherein q is independently 0, 1, 2, or 3 and substituents R″ each are independently selected from the group consisting of hydrogen, deuterium, fluorine, and chlorine” means that, in formula Q-1, there are q substituents R″ on the benzene ring, the substituents R″ can be the same or different, and options for each substituent R″ do not affect each other; and in formula Q-2, there are q substituents R″ on each benzene ring of the biphenyl, the numbers q of substituents R″ on the two benzene rings can be the same or different, the substituents R″ can be the same or different, and options for each substituent R″ do not affect each other.

In the present disclosure, the term “substituted or unsubstituted” means that a functional group after the term may have or may not have a substituent (hereinafter, for ease of description, substituents are collectively referred to as Rc). For example, the “substituted or unsubstituted aryl” refers to an aryl having one or more substituent Rc or unsubstituted aryl. For example, the substituents Rc each selected from the group consisting of deuterium, halogen, cyano, heteroaryl with 3 to 20 carbon atoms, aryl with 6 to 20 carbon atoms, alkyl with 1 to 10 carbon atoms, haloalkyl with 1 to 10 carbon atoms, deuterated alkyl with 1 to 10 carbon atoms, alkoxy with 1 to 10 carbon atoms, cycloalkyl with 3 to 10 carbon atoms, alkylthio with 1 to 10 carbon atoms, trialkylsilyl with 3 to 12 carbon atoms, arylsilyl with 6 to 18 carbon atoms, aryloxy with 6 to 20 carbon atoms, and arylthio with 6 to 20 carbon atoms. A substituted functional group may have one or more of the above-mentioned substituents Rc, wherein when two substituents Rc are connected to the same atom, these two substituents Rc may exist independently or connected to each other to form a ring with the atom to which they are jointly connected; and when there are two adjacent substituents Rc on the group, the two adjacent substituents Rc may exist independently or form a fused ring with the group.

In the present disclosure, the number of carbon atoms in a substituted or unsubstituted functional group refers to the number of all carbon atoms. For example, if L₃ is substituted arylene with 12 carbon atoms, the number of all carbon atoms in the arylene and substituents thereon is 12. For example, if Ar₁ is

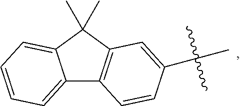 [SMILES: *c1ccc2c(c1)C(C)(C)c1ccccc1-2]

the number of carbon atoms in Ar₁ is 15; and if L₃ is

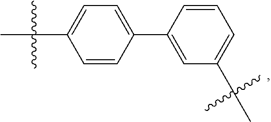 [SMILES: *c1ccc(-c2cccc(*)c2)cc1]

the number of carbon atoms in L₃ is 12.

In the present disclosure, the alkyl may include linear alkyl or branched alkyl. The alkyl may have 1 to 10 carbon atoms, and the numerical range “1 to 10” refers to any integer in the range. Optionally, the alkyl is alkyl with 1 to 4 carbon atoms, and specific examples may include, but are not limited to, methyl, ethyl, propyl, isopropyl, n-butyl, isobutyl, sec-butyl, and tert-butyl.

In the present disclosure, the cycloalkyl refers to saturated hydrocarbyl with an alicyclic structure, including monocyclic and fused-ring structures. The cycloalkyl may have 3 to 10 carbon atoms, and the numerical range “3 to 10” refers to any integer in the range. The cycloalkyl may have a spiro-ring system (with two rings sharing one carbon atom), a fused ring system (with two rings sharing two carbon atoms), or a bridged ring system (with two rings sharing three or more carbon atoms). Specific examples of cycloalkyl may include, but are not limited to, cyclohexyl, cyclopentyl, and adamantyl.

In the present disclosure, the aryl refers to any functional group or substituent derived from an aromatic carbocyclic ring. The aryl may refer to a monocyclic aryl group (such as phenyl) or a polycyclic aryl group. In other words, the aryl may refer a monocyclic aryl group, a fused-ring aryl group, two or more monocyclic aryl groups that are conjugated through carbon-carbon bonds, a monocyclic aryl group and a fused-ring aryl group that are conjugated through carbon-carbon bonds, and two or more fused-ring aryl groups that are conjugated through carbon-carbon bonds. That is, unless otherwise specified, two or more aromatic groups that are conjugated through carbon-carbon bonds can also be regarded as the aryl of the present disclosure. For example, the fused-ring aryl group may include a bicyclic fused aryl group (such as naphthyl) and a tricyclic fused aryl group (such as phenanthryl, fluorenyl, and anthracenyl). The aryl does not include heteroatoms such as B, N, O, S, P, Se, and Si. Examples of the aryl may include, but are not limited to, phenyl, naphthyl, fluorenyl, anthracenyl, phenanthryl, biphenyl, terphenyl, tetraphenyl, pentaphenyl, benzo[9,10]phenanthryl, pyrenyl, benzofluoranthenyl, and chrysenyl. The substituted or unsubstituted aryl of the present disclosure may include 6 to 30 carbon atoms. In some embodiments, the substituted or unsubstituted aryl may include 6 to 25 carbon atoms; in some embodiments, the substituted or unsubstituted aryl may include 6 to 20 carbon atoms; in some embodiments, the substituted or unsubstituted aryl may include 6 to 18 carbon atoms; and in some embodiments, the substituted or unsubstituted aryl may include 6 to 15 carbon atoms. For example, there can be 6, 10, 12, 13, 14, 15, 16, 18, 20, 24, 25, or 30 carbon atoms in the aryl, and there can also be any other number of carbon atoms in the aryl, which will not be listed here. In the present disclosure, the biphenyl can be construed as phenyl-substituted aryl, and can also be construed as unsubstituted aryl.

The arylene involved in the present disclosure refers to a divalent or multivalent group obtained after one or more hydrogen atoms are further removed from aryl.

In the present disclosure, the substituted aryl may refer to aryl in which one or more hydrogen atoms are substituted by a group such as deuterium, halogen, cyano, aryl, heteroaryl, trialkylsilyl, alkyl, cycloalkyl, or alkoxy. It should be understood that the number of carbon atoms in the substituted aryl refers to the total number of carbon atoms in the aryl and substituents thereon. For example, in substituted aryl with 18 carbon atoms, there are a total of 18 carbon atoms in the aryl and substituents thereon.

In the present disclosure, the fluorenyl is substituted, and two substituents are connected to each other to form a spiro structure. Specific examples of substituted fluorenyl may include, but are not limited to, the following structures:

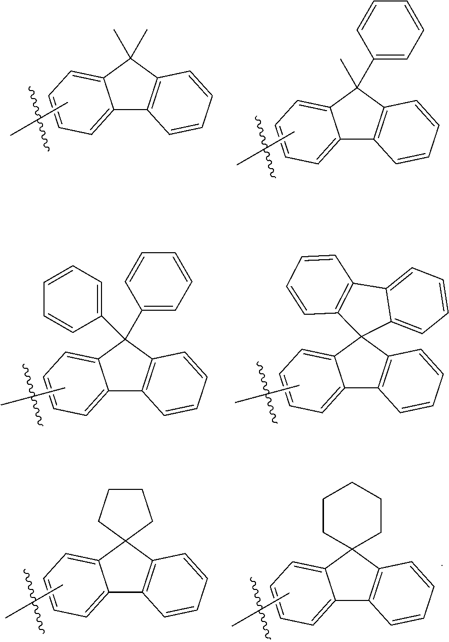

In the present disclosure, specific examples of aryl with 6 to 20 carbon atoms as a substituent may include, but are not limited to, phenyl, naphthyl, anthracenyl, phenanthryl, dimethylfluorenyl, and biphenyl.

In the present disclosure, the heteroaryl refers to a monovalent aromatic ring with 1, 2, 3, 4, 5, 6, 7, or more heteroatoms or a derivative thereof. The heteroatoms may be at least one selected from the group consisting of B, O, N, P, Si, Se, and S. The heteroaryl can be monocyclic heteroaryl or polycyclic heteroaryl. In other words, the heteroaryl may refer to a single aromatic ring system or multiple aromatic ring systems conjugated through carbon-carbon bonds, wherein each aromatic ring system is an aromatic monocyclic ring or an aromatic fused ring. For example, the heteroaryl may include, but is not limited to, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silylfluorenyl, dibenzofuranyl, N-arylcarbazolyl (such as N-phenylcarbazolyl), N-heteroarylcarbazolyl (such as N-pyridylcarbazolyl), and N-alkylcarbazolyl (such as N-methylcarbazolyl). The thienyl, furyl, phenanthrolinyl, and the like are heteroaryl with a single aromatic ring system; and the N-phenylcarbazolyl, N-pyridylcarbazolyl, and the like are heteroaryl with multiple ring systems conjugated through carbon-carbon bonds. The substituted or unsubstituted heteroaryl of the present disclosure may include 3 to 30 carbon atoms. In some embodiments, the substituted or unsubstituted heteroaryl may include 5 to 25 carbon atoms; in some embodiments, the substituted or unsubstituted heteroaryl may include 5 to 20 carbon atoms; in some embodiments, the substituted or unsubstituted heteroaryl may include 5 to 18 carbon atoms; and in some embodiments, the substituted or unsubstituted heteroaryl may include 5 to 12 carbon atoms. For example, there can be 3, 4, 5, 7, 12, 13, 14, 15, 16, 18, 20, 24, 25, or 30 carbon atoms in the substituted or unsubstituted heteroaryl, and there can also be any other number of carbon atoms in the substituted or unsubstituted heteroaryl, which will not be listed here.

The heteroarylene involved in the present disclosure refers to a divalent or multivalent group obtained after one or more hydrogen atoms are further removed from heteroaryl.

In the present disclosure, the substituted heteroaryl may refer to heteroaryl in which one or more hydrogen atoms are substituted by a group such as deuterium, halogen, cyano, aryl, heteroaryl, trialkylsilyl, alkyl, cycloalkyl, or alkoxy. It should be understood that the number of carbon atoms in the substituted heteroaryl refers to the total number of carbon atoms in the heteroaryl and substituents thereon.

In the present disclosure, specific examples of heteroaryl with 3 to 20 carbon atoms as a substituent may include, but are not limited to, carbazolyl, dibenzofuranyl, dibenzothienyl, pyridyl, quinolinyl, isoquinolinyl, quinoxalinyl, and quinazolinyl.

In the present disclosure, the halogen may include fluorine, iodine, bromine, chlorine, or the like.

In the present disclosure, specific examples of the trialkylsilyl with 3 to 12 carbon atoms may include, but are not limited to, trimethylsilyl and triethylsilyl.

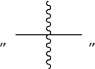

In the present disclosure, a non-positional bond refers to a single bond extending from a ring system, which means that one end of the bond can be attached to any position in the ring system through which the bond penetrates, and the other end is attached to the remaining part in the compound molecule. For example, as shown in the following formula (f), the naphthyl represented by the formula (f) is attached to the remaining part in the molecule through two non-positional bonds that penetrate through the bicyclic ring, which indicates any possible attachment modes shown in formula (f-1) to formula (f-10).

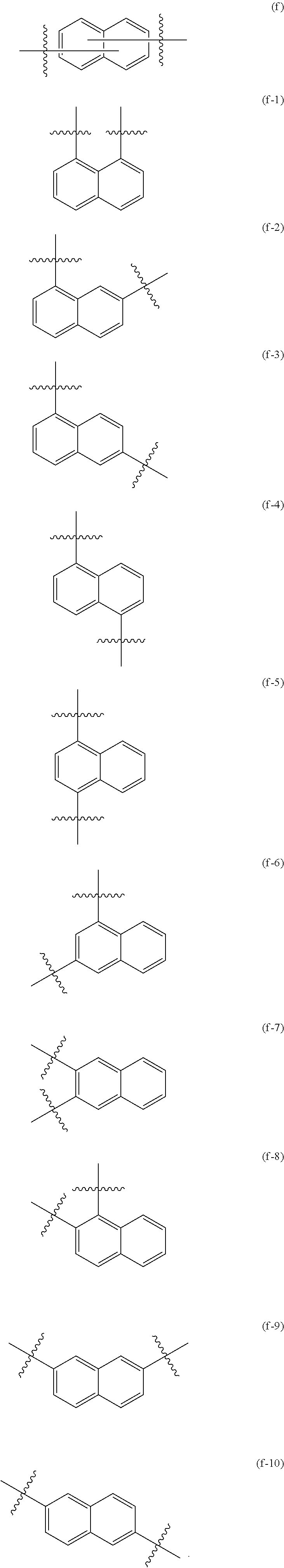

For example, as shown in the following formula (X′), the dibenzofuranyl represented by the formula (X′) is attached to the remaining part in the molecule through a non-positional bond extending from the middle of a benzene ring at a side, which indicates any possible attachment modes shown in formula (X′-1) to formula (X′-4).

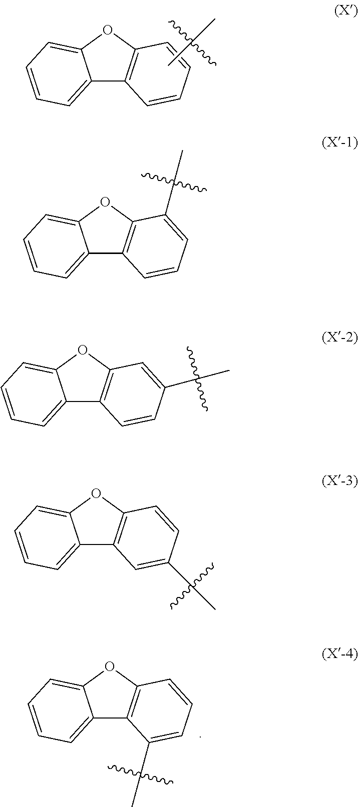

In the present disclosure, a non-positional substituent refers to a substituent linked through a single bond extending from the center of a ring system, which means that the substituent can be attached to any possible position in the ring system. For example, as shown in the following formula (Y), the substituent R′ represented by the formula (Y) is attached to a quinoline ring through a non-positional bond, which indicates any possible attachment modes shown in formula (Y-1) to formula (Y-7).

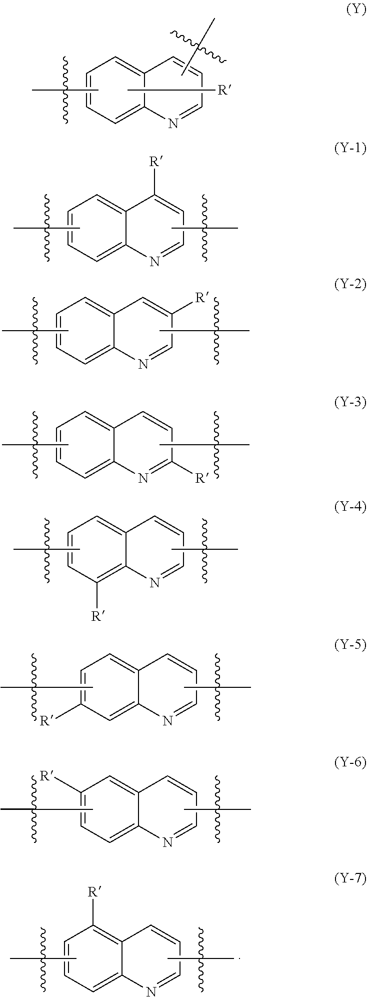

In one embodiment of the present disclosure, L₁, L₂, and L₃ are the same or different, and are each independently selected from the group consisting of a single bond, substituted or unsubstituted arylene with 6 to 18 carbon atoms, and substituted or unsubstituted heteroarylene with 5 to 18 carbon atoms.

Optionally, substituents in L₁, L₂, and L₃ are the same or different, and are each independently selected from the group consisting of deuterium, halogen, cyano, phenyl, trialkylsilyl with 3 to 8 carbon atoms, alkyl with 1 to 4 carbon atoms, haloalkyl with 1 to 4 carbon atoms, deuterated alkyl with 1 to 4 carbon atoms, alkoxy with 1 to 4 carbon atoms, alkylthio with 1 to 4 carbon atoms, phenyl, naphthyl, biphenyl, anthracenyl, phenanthryl, pyridyl, dibenzothienyl, dibenzofuranyl, and carbazolyl.

In one embodiment of the present disclosure, L₁, L₂, and L₃ are each independently selected from the group consisting of a single bond, substituted or unsubstituted phenylene, substituted or unsubstituted naphthylene, substituted or unsubstituted biphenylene, substituted or unsubstituted anthracenylene, substituted or unsubstituted phenanthrylene, substituted or unsubstituted dibenzofuranylene, substituted or unsubstituted dibenzothienylene, substituted or unsubstituted fluorenylene, substituted or unsubstituted carbazolylene, and a group obtained by linking two or three of the above groups through a single bond; and

optionally, substituents in L₁, L₂, and L₃ are each independently selected from the group consisting of deuterium, fluorine, cyano, methyl, ethyl, n-propyl, isopropyl, tert-butyl, trifluoromethyl, trideuteromethyl, trimethylsilyl, cyclopentyl, phenyl, biphenyl, naphthyl, anthracenyl, phenanthryl, dibenzothienyl, dibenzofuranyl, carbazolyl, and pyridyl.

In one embodiment of the present disclosure, L₁, L₂, and L₃ are each independently selected from the group consisting of a single bond and a substituted or unsubstituted group V; an unsubstituted group V are selected from the group consisting of the following groups:

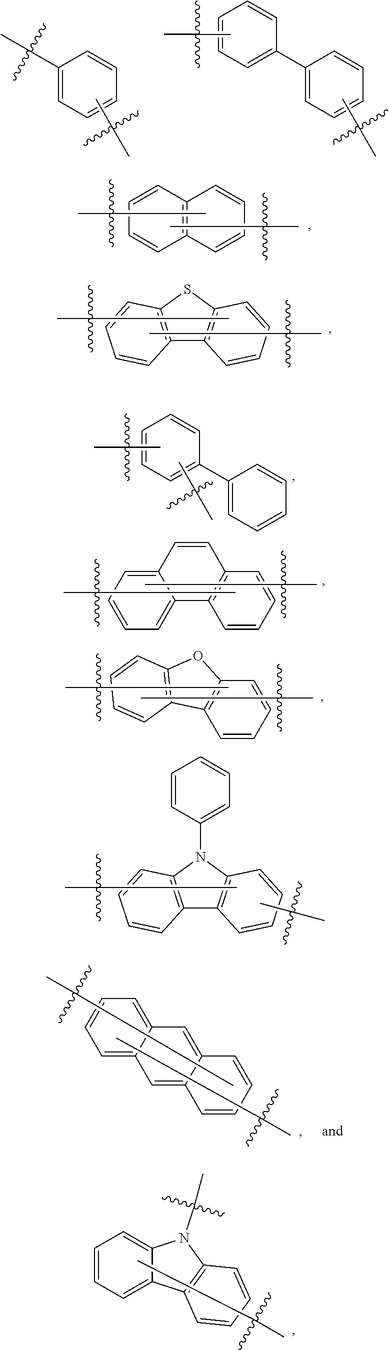

wherein, [symbol] represents a chemical bond; a substituted group V may have one or more substituents, and the one or more substituents are each independently selected from the group consisting of deuterium, cyano, fluorine, methyl, ethyl, n-propyl, isopropyl, tert-butyl, trifluoromethyl, trideuteromethyl, trimethylsilyl, phenyl, naphthyl, and pyridyl; and when the group V has two or more substituents, the two or more substituents are the same or different.

In the present disclosure, the “more” refers to two or more.

Optionally, L₁, L₂, and L₃ are each independently selected from the group consisting of a single bond and the following groups:

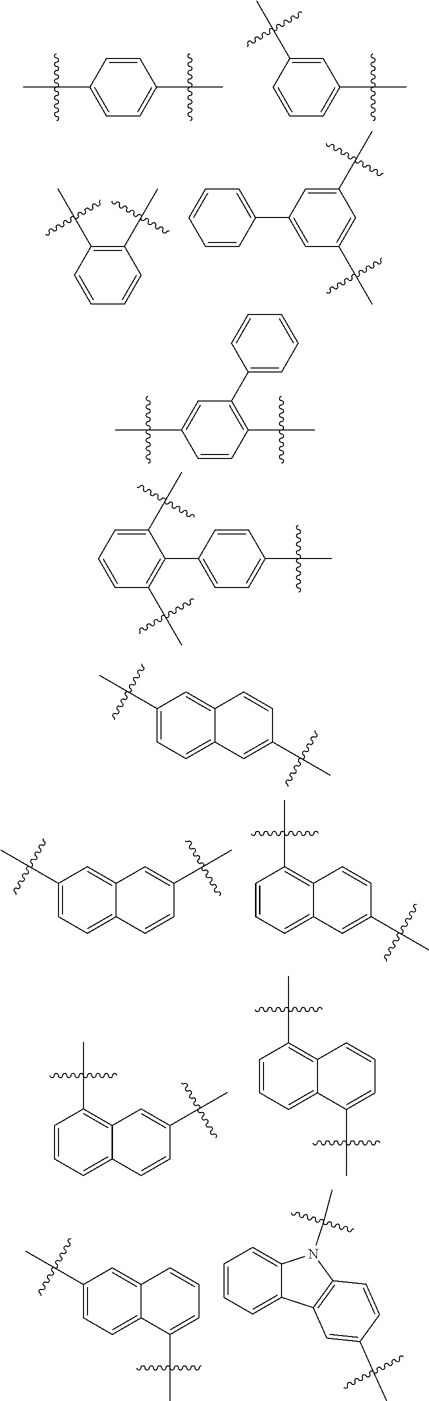

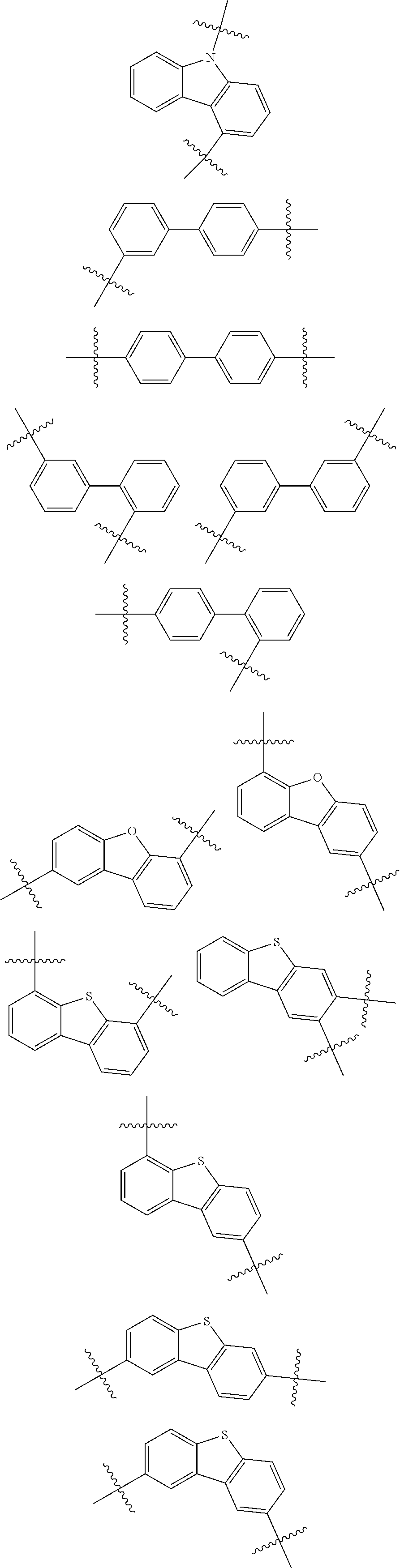

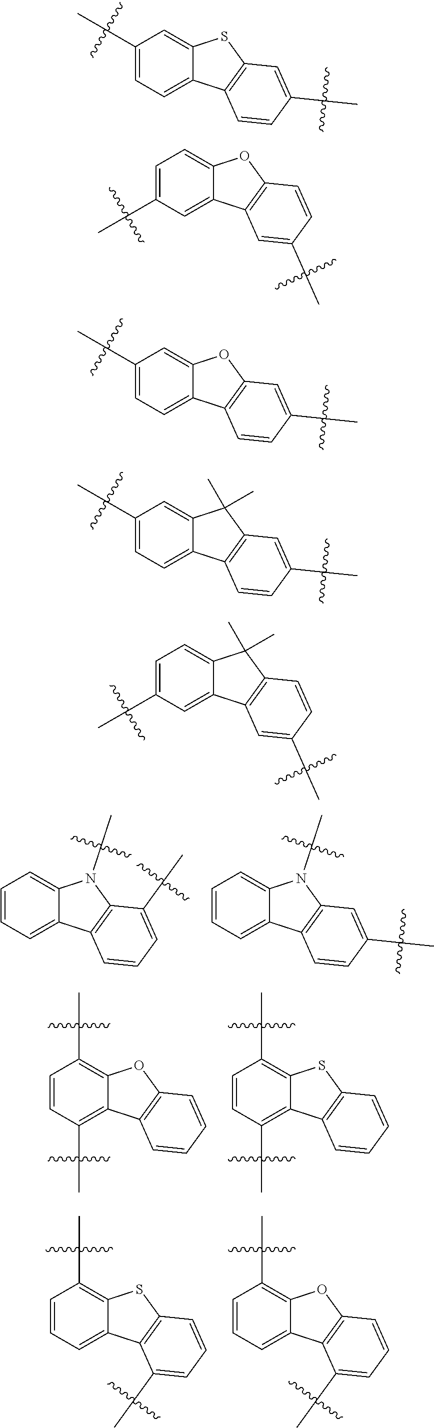

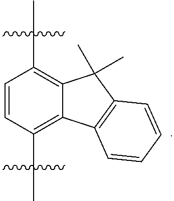 [SMILES: *c1ccc(*)c2c1-c1ccccc1C2(C)C]

In one embodiment of the present disclosure, Ar₁ and Ar₂ are the same or different, and are each independently selected from the group consisting of substituted or unsubstituted aryl with 6 to 25 carbon atoms and substituted or unsubstituted heteroaryl with 5 to 25 carbon atoms.

Optionally, substituents in Ar₁ and Ar₂ are each independently selected from the group consisting of deuterium, halogen, cyano, aryl with 6 to 15 carbon atoms, heteroaryl with 5 to 12 carbon atoms, alkyl with 1 to 4 carbon atoms, trialkylsilyl with 3 to 8 carbon atoms, cycloalkyl with 5 to 10 carbon atoms, haloalkyl with 1 to 4 carbon atoms, deuterated alkyl with 1 to 4 carbon atoms, alkoxy with 1 to 4 carbon atoms, and alkylthio with 1 to 4 carbon atoms.

Optionally, substituents in Ar₁ and Ar₂ are each independently selected from the group consisting of deuterium, cyano, fluorine, methyl, ethyl, n-propyl, isopropyl, tert-butyl, trifluoromethyl, trideuteromethyl, trimethylsilyl, methoxy, isopropoxy, phenyl, cyclohexyl, phenyl, naphthyl, fluorenyl, dibenzothienyl, dibenzofuranyl, phenanthryl, and carbazolyl.

Optionally, Ar₁ and Ar₂ are each independently selected from the group consisting of substituted or unsubstituted phenyl, substituted or unsubstituted biphenyl, substituted or unsubstituted terphenyl, substituted or unsubstituted naphthyl, substituted or unsubstituted phenanthryl, substituted or unsubstituted anthracenyl, substituted or unsubstituted dibenzofuranyl, substituted or unsubstituted dibenzothienyl, substituted or unsubstituted fluorenyl, substituted or unsubstituted carbazolyl, substituted or unsubstituted pyridyl, substituted or unsubstituted spirobifluorenyl, substituted or unsubstituted phenothiazinyl, and substituted or unsubstituted phenoxthiyl.

Optionally, Ar₁ and Ar₂ are each independently a substituted or unsubstituted group W; an unsubstituted group W are selected from the group consisting of the following groups:

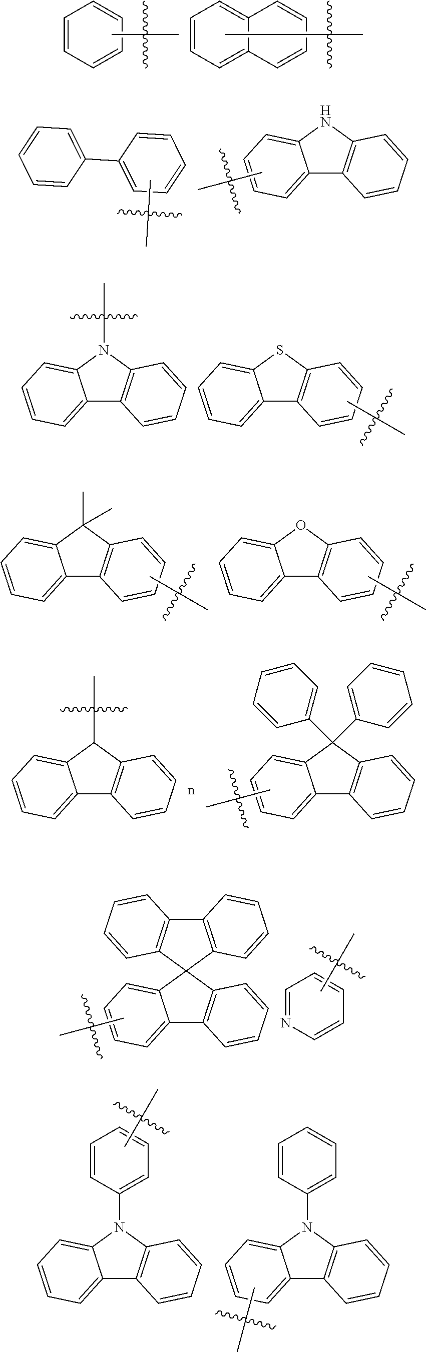

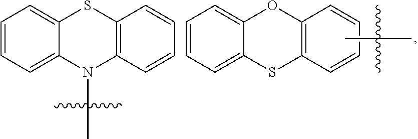

wherein,

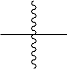

represents a chemical bond; a substituted group W may have one or more substituents, and the one or more substituents are each independently selected from the group consisting of deuterium, cyano, fluorine, methyl, ethyl, n-propyl, isopropyl, tert-butyl, trifluoromethyl, trideuteromethyl, trimethylsilyl, methoxy, isopropoxy, phenyl, cyclohexyl, phenyl, naphthyl, fluorenyl, dibenzothienyl, dibenzofuranyl, phenanthryl, and carbazolyl; and when the group W has two or more substituents, the two or more substituents are the same or different.

Further optionally, Ar₁ and Ar₂ are each independently selected from the group consisting of the following groups:

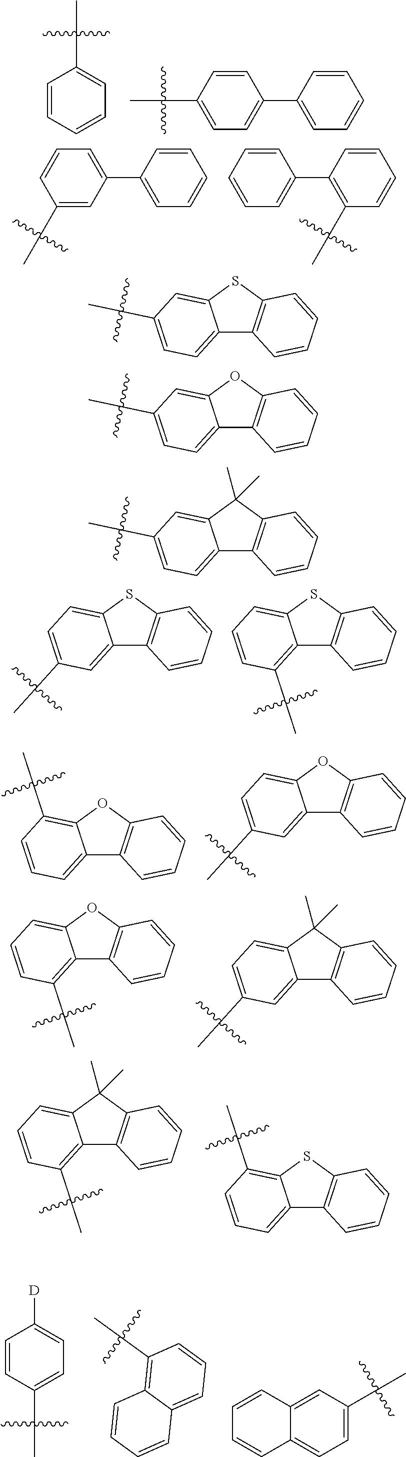

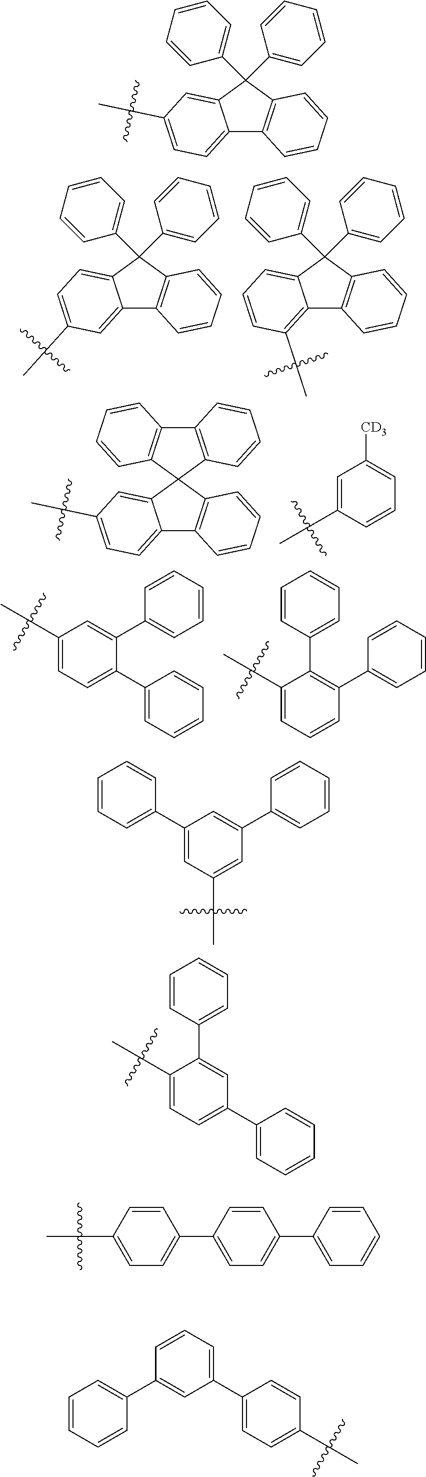

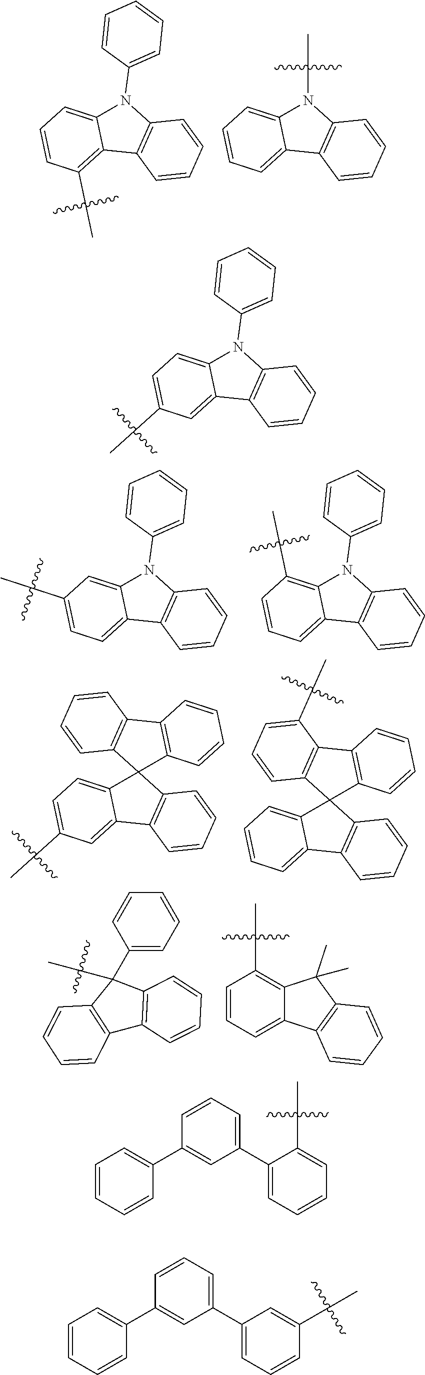

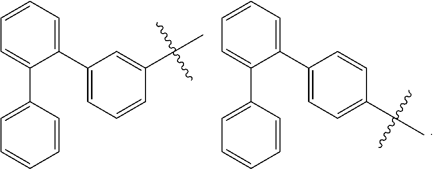 [SMILES: *c1cccc(-c2ccccc2-c2ccccc2)c1 ; *c1ccc(-c2ccccc2-c2ccccc2)cc1]

In one embodiment of the present disclosure, R₁, R₂, R₃, and R₄ are each independently selected from the group consisting of hydrogen, a group shown in chemical formula 2, deuterium, cyano, fluorine, methyl, ethyl, isopropyl, tert-butyl, trifluoromethyl, trideuteromethyl, trimethylsilyl, phenyl, naphthyl, biphenyl, dimethylfluorenyl, pyridyl, pyrimidinyl, quinolinyl, isoquinolinyl, carbazolyl, dibenzofuranyl, dibenzothienyl, dimethylfluorenyl, and N-phenylcarbazolyl, and only one of R₁, R₂, R₃, and R₄ is the group shown in chemical formula 2.

In one embodiment of the present disclosure, R₅ and R₆ are each independently selected from the group consisting of alkyl with 1 to 4 carbon atoms and aryl with 6 to 12 carbon atoms; or R₅ and R₆ are connected to each other to form an unsubstituted 5- to 10-membered aliphatic ring or a substituted or unsubstituted 9- to 14-membered aromatic ring together with carbon atoms attached to the two, and a substituent in the 9- to 14-membered aromatic ring are independently selected from the group consisting of deuterium, halogen, cyano, and alkyl with 1 to 4 carbon atoms.

In one embodiment of the present disclosure, R₅ and R₆ are each independently selected from the group consisting of methyl, ethyl, n-propyl, isopropyl, tert-butyl, trifluoromethyl, trideuteromethyl, phenyl, naphthyl, biphenyl, terphenyl, fluorenyl, dimethylfluorenyl, anthracenyl, phenanthryl, pyridyl, dibenzothienyl, dibenzofuranyl, and carbazolyl; or R₅ and R₆ are optionally connected to each other to form a fluorene ring, cyclopentane, cyclohexane, or

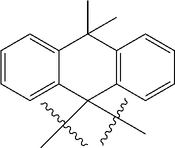 [SMILES: *C1(*)c2ccccc2C(C)(C)c2ccccc21]

together with the carbon atom to which they are jointly connected.

In one embodiment of the present disclosure, R₅ and R₆ are each independently selected from the group consisting of methyl and the following groups:

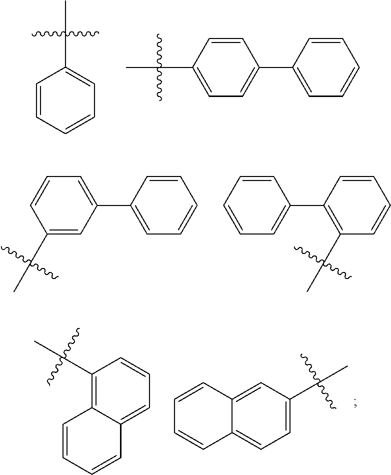

or R₅ and R₆ are optionally connected to form the following spiro-ring together with the carbon atom to which they are jointly connected:

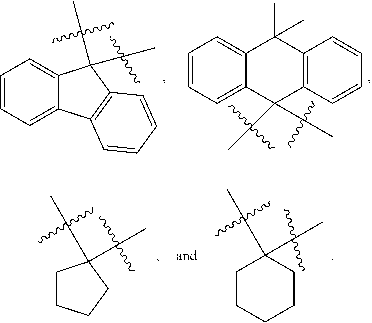

Optionally, the organic compound is selected from the group consisting of the following compounds:

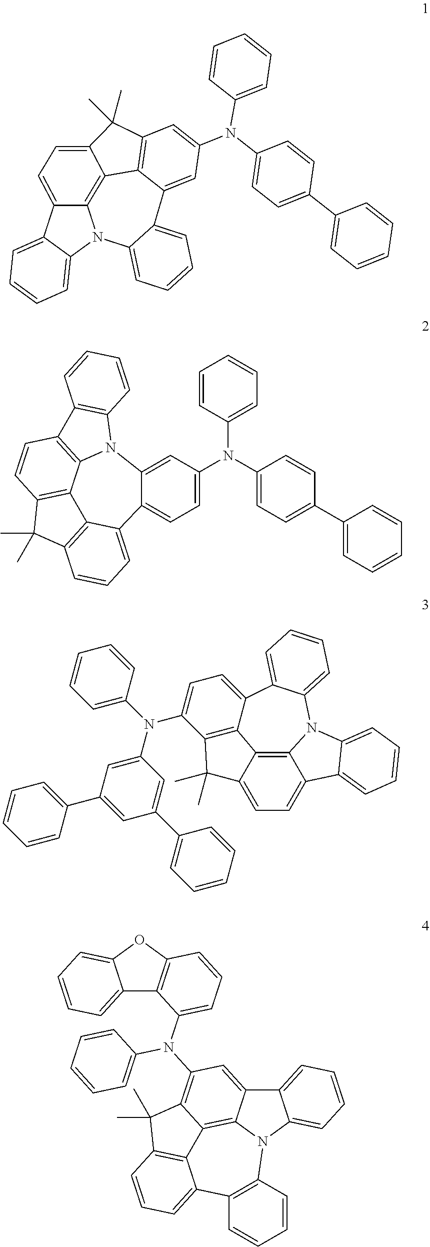 [SMILES: CC1(C)c2cc(N(c3ccccc3)c3ccc(-c4ccccc4)cc3)cc3c2-c2c1ccc1c4ccccc4n(c21)-c1ccccc1-3 ; CC1(C)c2cccc3c2-c2c1ccc1c4ccccc4n(c21)-c1cc(N(c2ccccc2)c2ccc(-c4ccccc4)cc2)ccc1-3 ; CC1(C)c2ccc3c4ccccc4n4c3c2-c2c(ccc(N(c3ccccc3)c3cc(-c5ccccc5)cc(-c5ccccc5)c3)c21)-c1ccccc1-4 ; CC1(C)c2cccc3c2-c2c1c(N(c1ccccc1)c1cccc4oc5ccccc5c14)cc1c4ccccc4n(c21)-c1ccccc1-3]

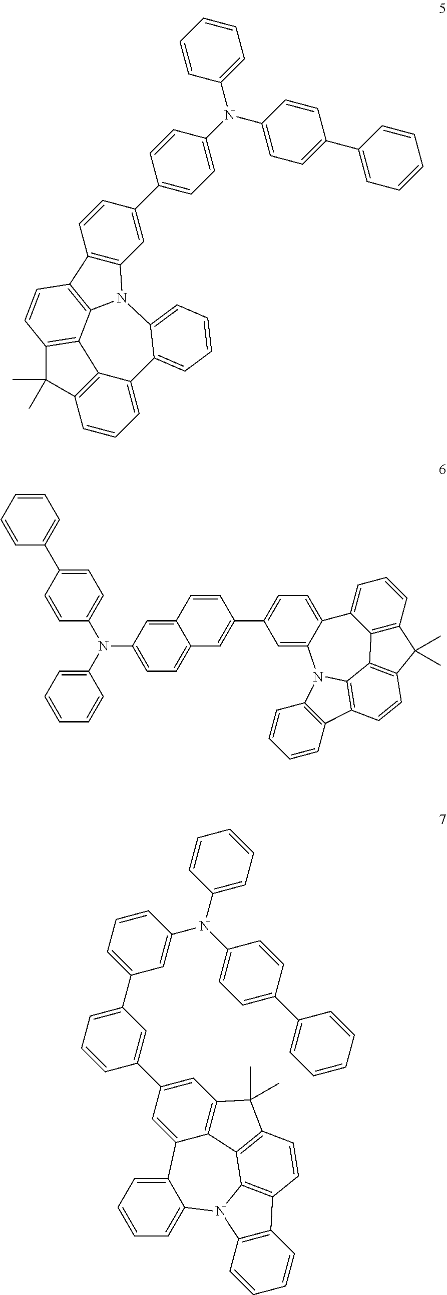 [SMILES: CC1(C)c2cccc3c2-c2c1ccc1c4ccc(-c5ccc(N(c6ccccc6)c6ccc(-c7ccccc7)cc6)cc5)cc4n(c21)-c1ccccc1-3 ; CC1(C)c2cccc3c2-c2c1ccc1c4ccccc4n(c21)-c1cc(-c2ccc4cc(N(c5ccccc5)c5ccc(-c6ccccc6)cc5)ccc4c2)ccc1-3 ; CC1(C)c2cc(-c3cccc(-c4cccc(N(c5ccccc5)c5ccc(-c6ccccc6)cc5)c4)c3)cc3c2-c2c1ccc1c4ccccc4n(c21)-c1ccccc1-3]

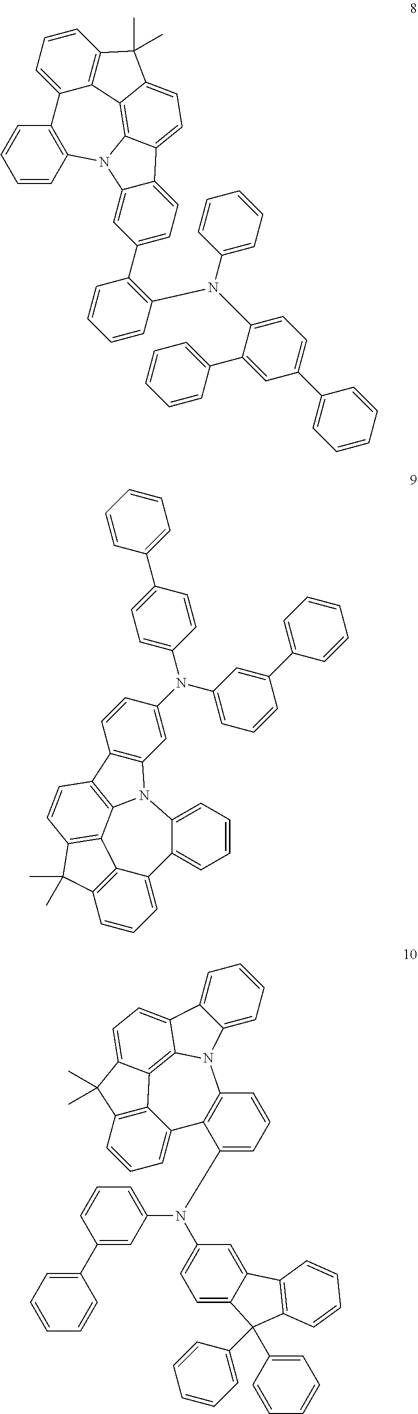 [SMILES: CC1(C)c2cccc3c2-c2c1ccc1c4ccc(-c5ccccc5N(c5ccccc5)c5ccc(-c6ccccc6)cc5-c5ccccc5)cc4n(c21)-c1ccccc1-3 ; CC1(C)c2cccc3c2-c2c1ccc1c4ccc(N(c5ccc(-c6ccccc6)cc5)c5cccc(-c6ccccc6)c5)cc4n(c21)-c1ccccc1-3 ; CC1(C)c2cccc3c2-c2c1ccc1c4ccccc4n(c21)-c1cccc(N(c2cccc(-c4ccccc4)c2)c2ccc4c(c2)-c2ccccc2C4(c2ccccc2)c2ccccc2)c1-3]

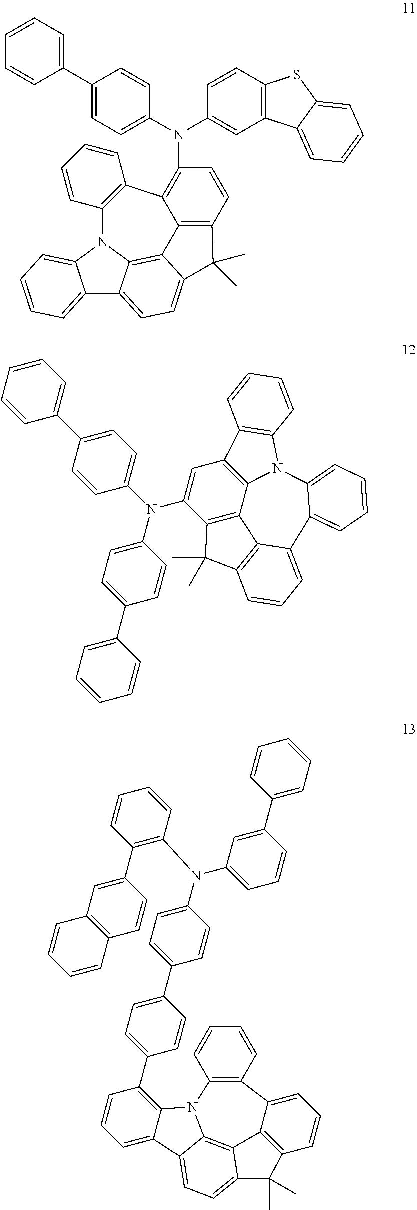

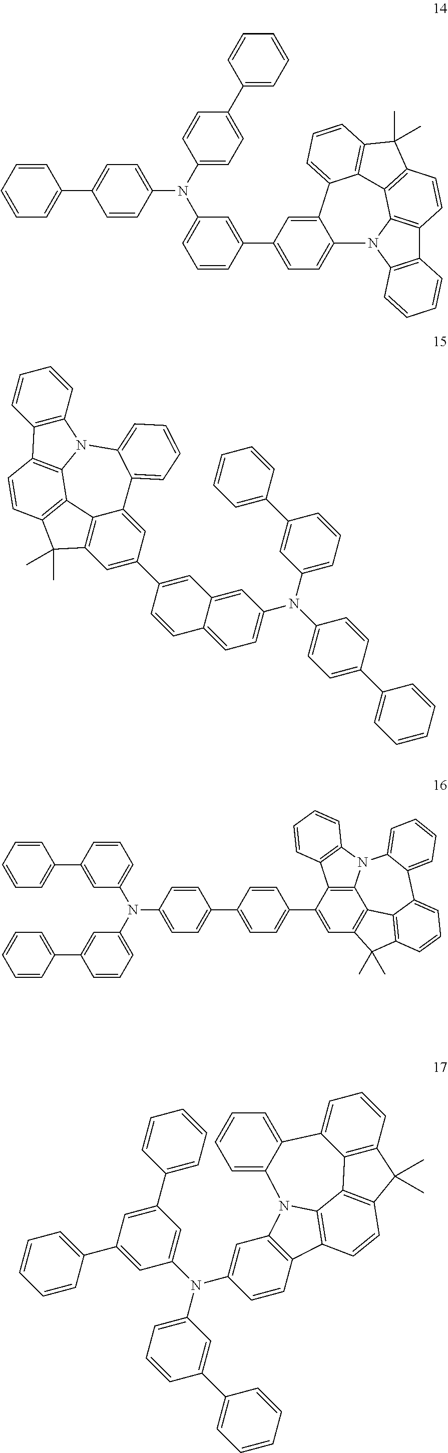 [SMILES: CC1(C)c2cccc3c2-c2c1ccc1c4ccccc4n(c21)-c1ccc(-c2cccc(N(c4ccc(-c5ccccc5)cc4)c4ccc(-c5ccccc5)cc4)c2)cc1-3 ; CC1(C)c2cc(-c3ccc4ccc(N(c5ccc(-c6ccccc6)cc5)c5cccc(-c6ccccc6)c5)cc4c3)cc3c2-c2c1ccc1c4ccccc4n(c21)-c1ccccc1-3 ; CC1(C)c2cccc3c2-c2c1cc(-c1ccc(-c4ccc(N(c5cccc(-c6ccccc6)c5)c5cccc(-c6ccccc6)c5)cc4)cc1)c1c4ccccc4n(c21)-c1ccccc1-3 ; CC1(C)c2cccc3c2-c2c1ccc1c4ccc(N(c5cccc(-c6ccccc6)c5)c5cc(-c6ccccc6)cc(-c6ccccc6)c5)cc4n(c21)-c1ccccc1-3]

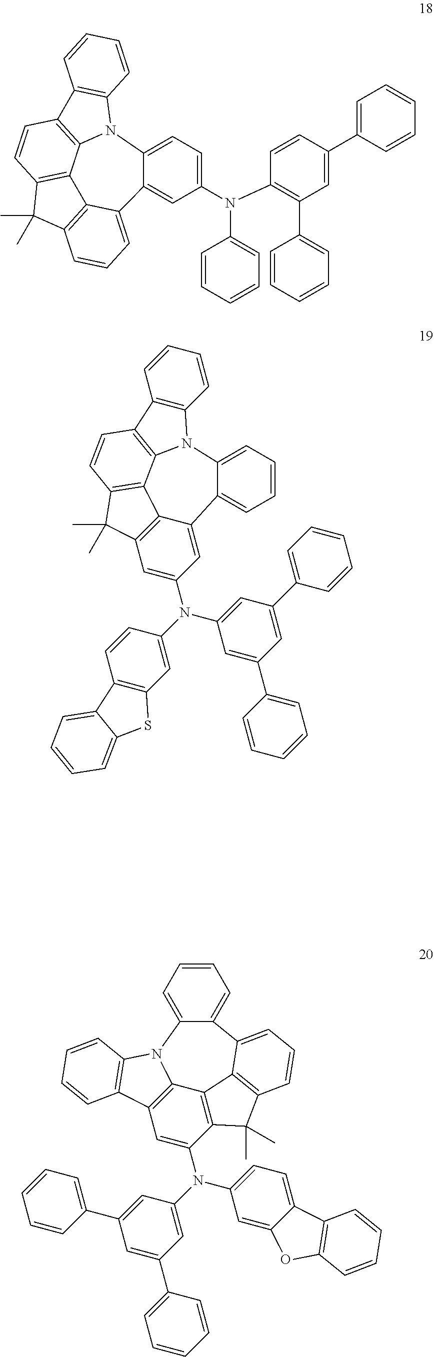 [SMILES: CC1(C)c2cccc3c2-c2c1ccc1c4ccccc4n(c21)-c1ccc(N(c2ccccc2)c2ccc(-c4ccccc4)cc2-c2ccccc2)cc1-3 ; CC1(C)c2cc(N(c3cc(-c4ccccc4)cc(-c4ccccc4)c3)c3ccc4c(c3)sc3ccccc34)cc3c2-c2c1ccc1c4ccccc4n(c21)-c1ccccc1-3 ; CC1(C)c2cccc3c2-c2c1c(N(c1cc(-c4ccccc4)cc(-c4ccccc4)c1)c1ccc4c(c1)oc1ccccc14)cc1c4ccccc4n(c21)-c1ccccc1-3]

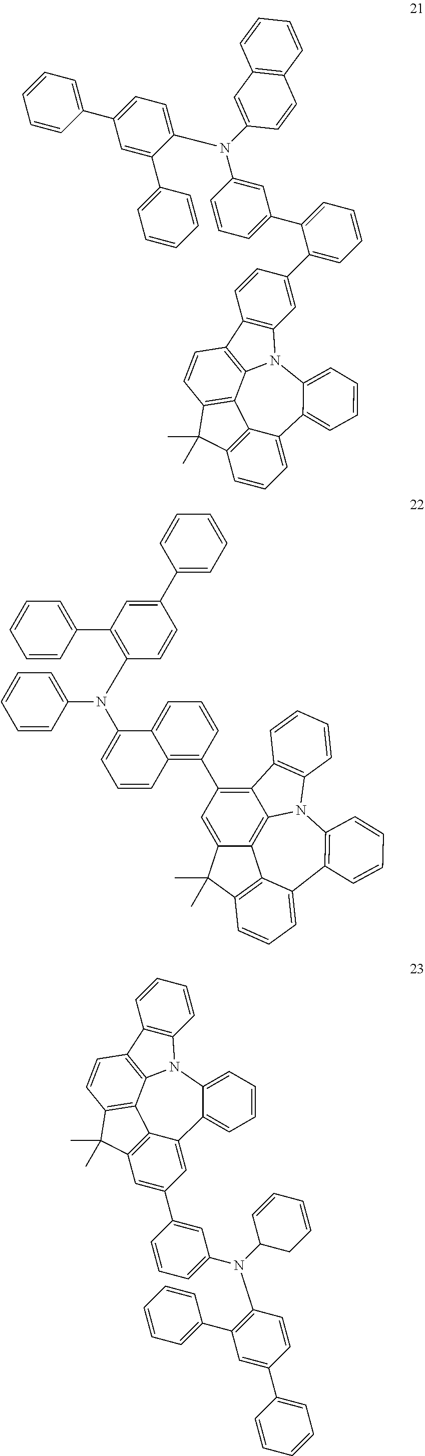

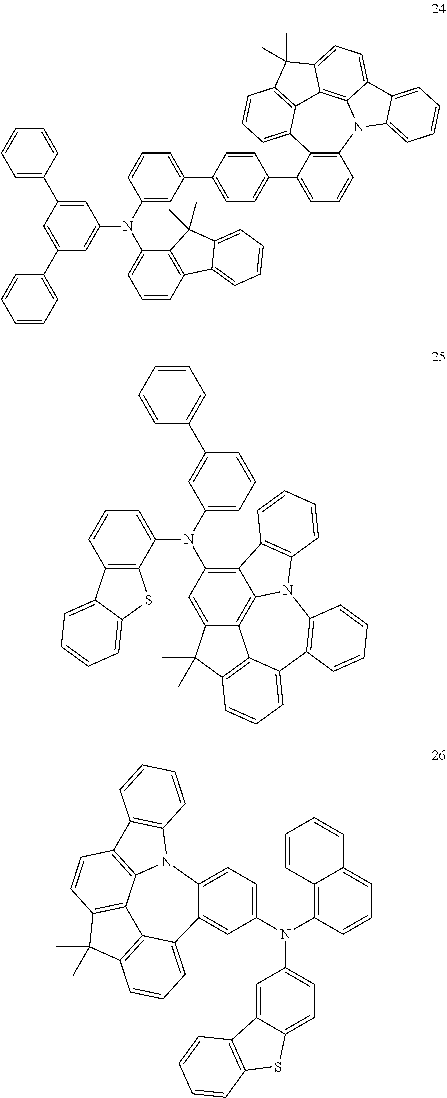 [SMILES: CC1(C)c2cccc3c2-c2c1ccc1c4ccccc4n(c21)-c1cccc(-c2ccc(-c4cccc(N(c5cc(-c6ccccc6)cc(-c6ccccc6)c5)c5cccc6c5C(C)(C)c5ccccc5-6)c4)cc2)c1-3 ; CC1(C)c2cccc3c2-c2c1cc(N(c1cccc(-c4ccccc4)c1)c1cccc4c1sc1ccccc14)c1c4ccccc4n(c21)-c1ccccc1-3 ; CC1(C)c2cccc3c2-c2c1ccc1c4ccccc4n(c21)-c1ccc(N(c2ccc4sc5ccccc5c4c2)c2cccc4ccccc24)cc1-3]

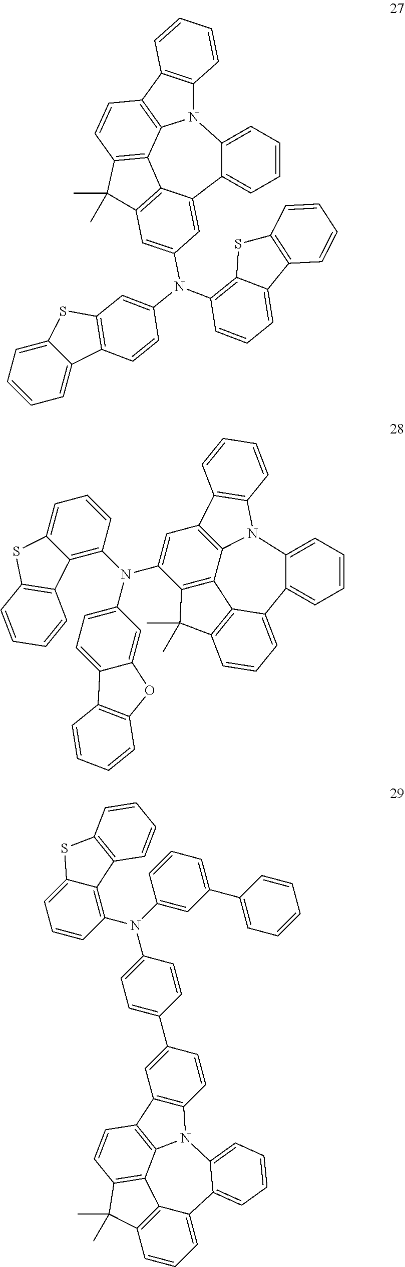

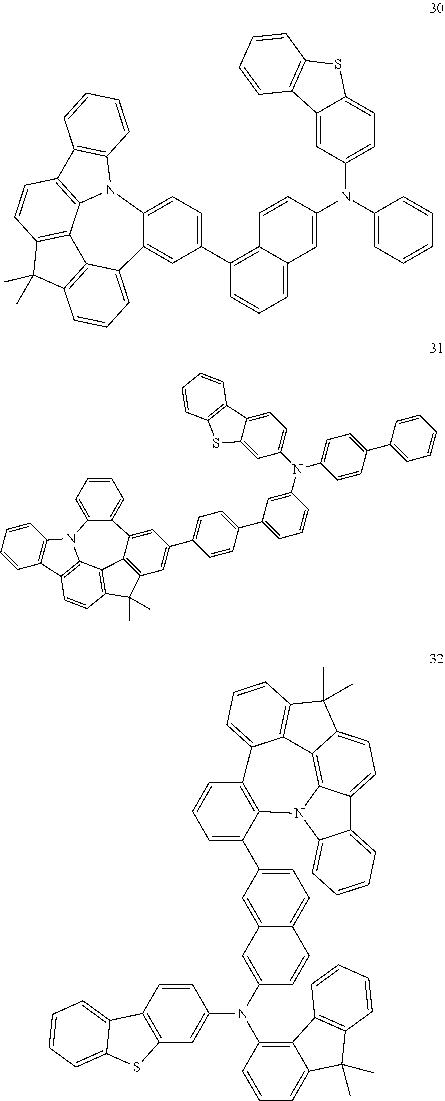 [SMILES: CC1(C)c2cccc3c2-c2c1ccc1c4ccccc4n(c21)-c1ccc(-c2cccc4cc(N(c5ccccc5)c5ccc6sc7ccccc7c6c5)ccc24)cc1-3 ; CC1(C)c2cc(-c3ccc(-c4cccc(N(c5ccc(-c6ccccc6)cc5)c5ccc6c(c5)sc5ccccc56)c4)cc3)cc3c2-c2c1ccc1c4ccccc4n(c21)-c1ccccc1-3 ; CC1(C)c2ccccc2-c2c(N(c3ccc4ccc(-c5cccc6c5-n5c7ccccc7c7ccc8c(c75)-c5c-6cccc5C8(C)C)cc4c3)c3ccc4c(c3)sc3ccccc34)cccc21]

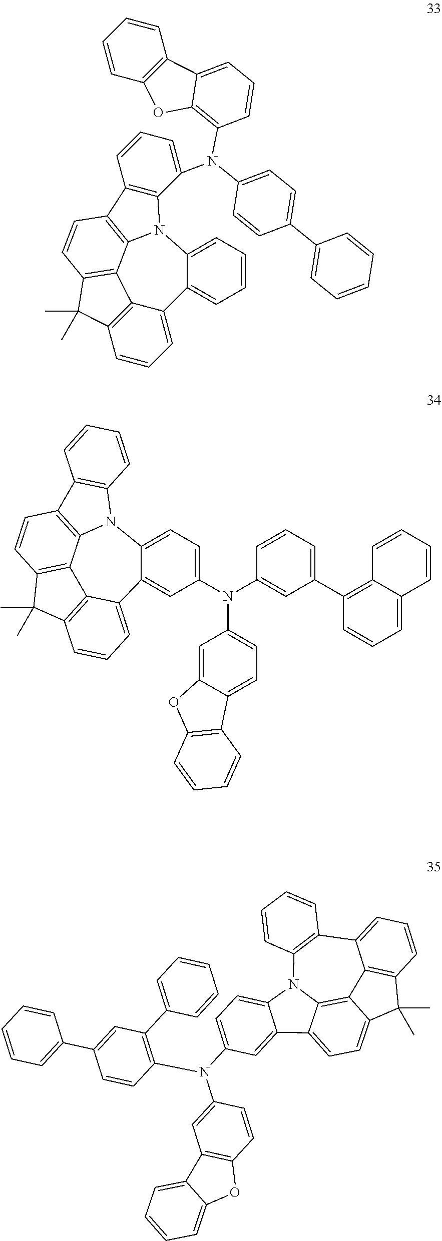 [SMILES: CC1(C)c2cccc3c2-c2c1ccc1c4cccc(N(c5ccc(-c6ccccc6)cc5)c5cccc6c5oc5ccccc56)c4n(c21)-c1ccccc1-3 ; CC1(C)c2cccc3c2-c2c1ccc1c4ccccc4n(c21)-c1ccc(N(c2cccc(-c4cccc5ccccc45)c2)c2ccc4c(c2)oc2ccccc24)cc1-3 ; CC1(C)c2cccc3c2-c2c1ccc1c4cc(N(c5ccc6oc7ccccc7c6c5)c5ccc(-c6ccccc6)cc5-c5ccccc5)ccc4n(c21)-c1ccccc1-3]

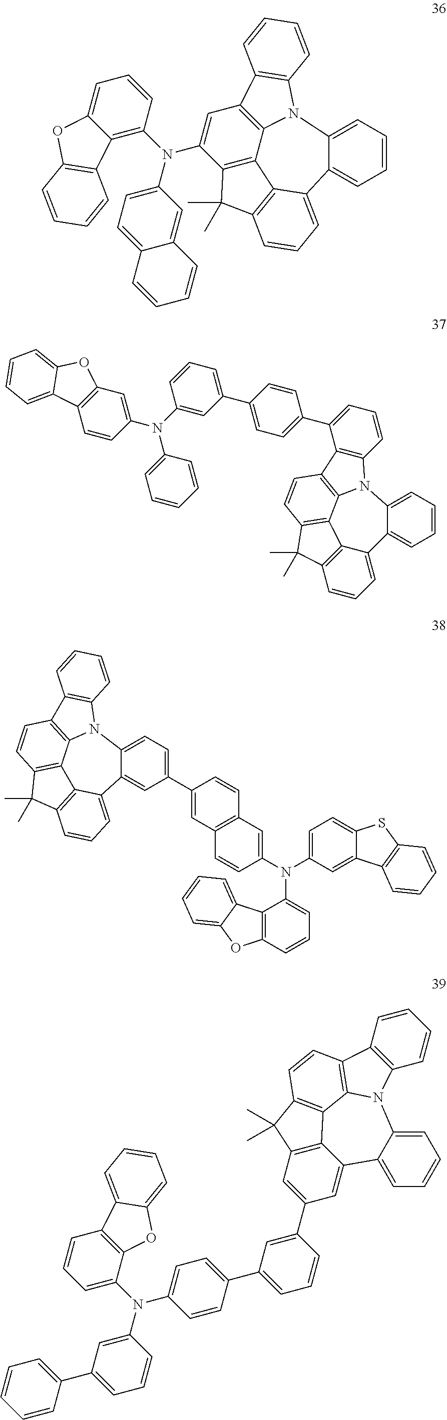 [SMILES: CC1(C)c2cccc3c2-c2c1c(N(c1ccc4ccccc4c1)c1cccc4oc5ccccc5c14)cc1c4ccccc4n(c21)-c1ccccc1-3 ; CC1(C)c2cccc3c2-c2c1ccc1c4c(-c5ccc(-c6cccc(N(c7ccccc7)c7ccc8c(c7)oc7ccccc78)c6)cc5)cccc4n(c21)-c1ccccc1-3 ; CC1(C)c2cccc3c2-c2c1ccc1c4ccccc4n(c21)-c1ccc(-c2ccc4cc(N(c5ccc6sc7ccccc7c6c5)c5cccc6oc7ccccc7c56)ccc4c2)cc1-3 ; CC1(C)c2cc(-c3cccc(-c4ccc(N(c5cccc(-c6ccccc6)c5)c5cccc6c5oc5ccccc56)cc4)c3)cc3c2-c2c1ccc1c4ccccc4n(c21)-c1ccccc1-3]

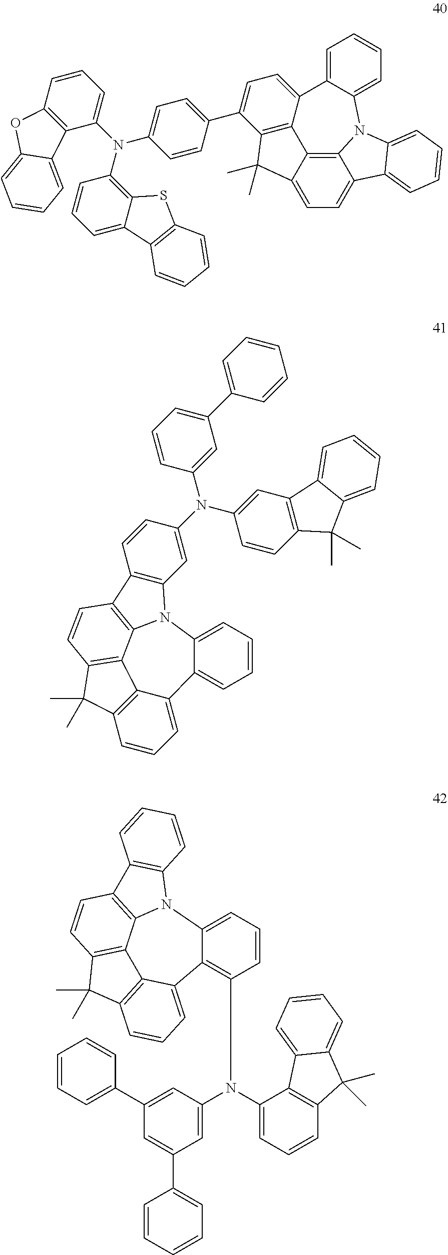

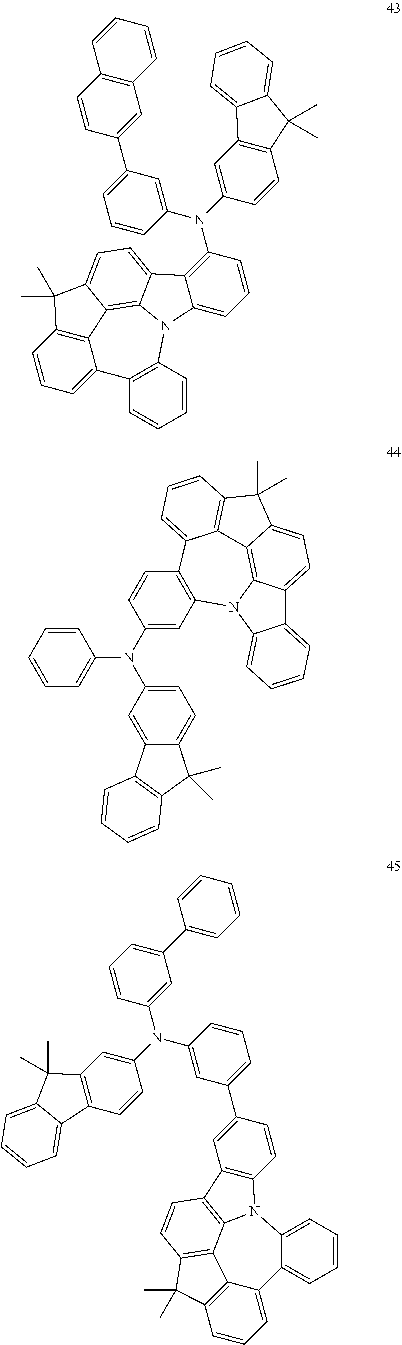

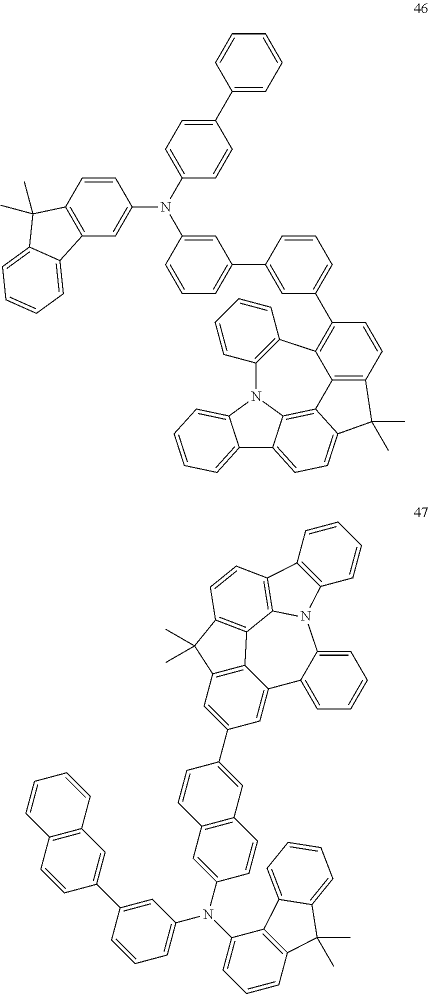

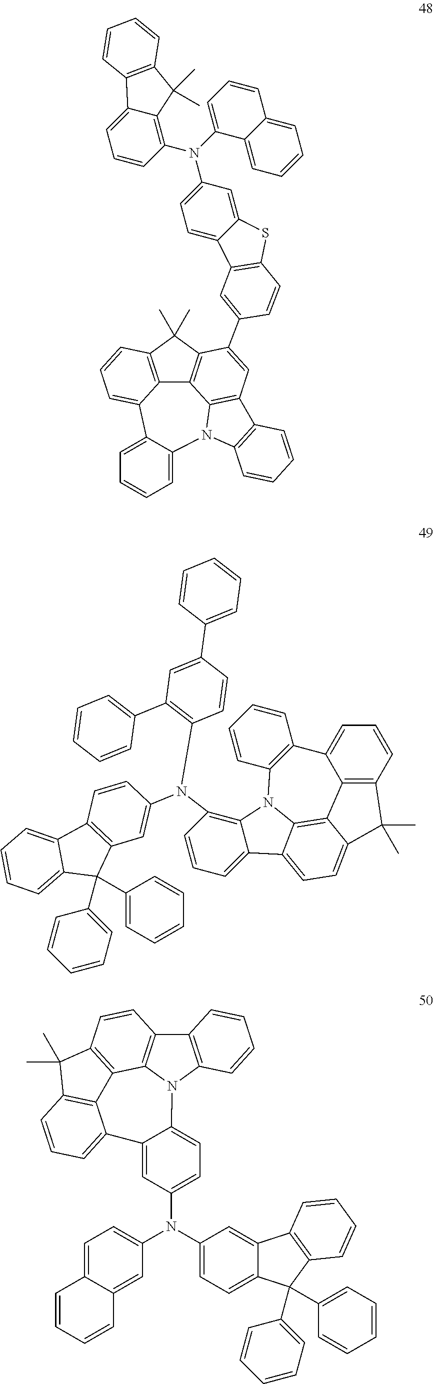

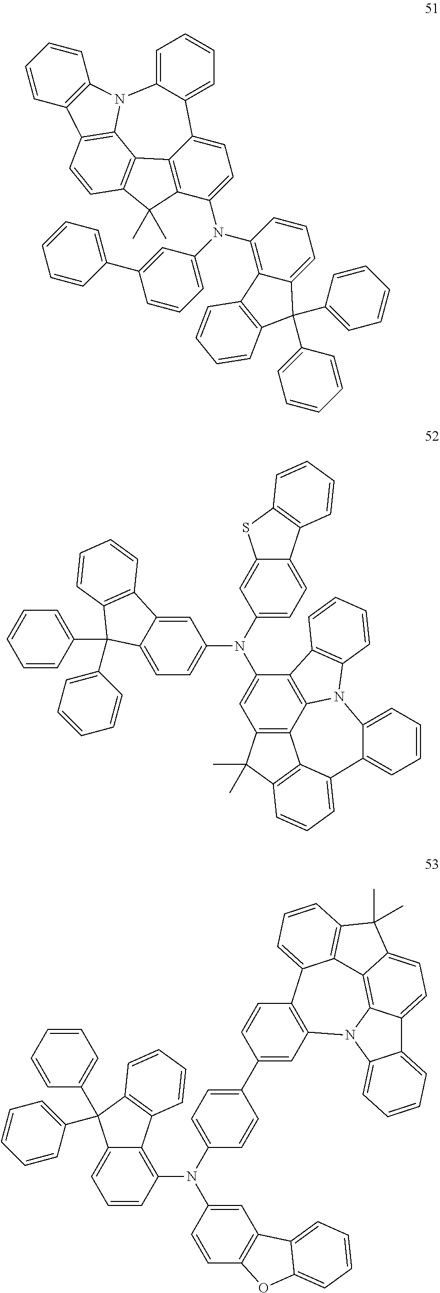 [SMILES: CC1(C)c2ccc3c4ccccc4n4c3c2-c2c(ccc(N(c3cccc(-c5ccccc5)c3)c3cccc5c3-c3ccccc3C5(c3ccccc3)c3ccccc3)c21)-c1ccccc1-4 ; CC1(C)c2cccc3c2-c2c1cc(N(c1ccc4c(c1)-c1ccccc1C4(c1ccccc1)c1ccccc1)c1ccc4c(c1)sc1ccccc14)c1c4ccccc4n(c21)-c1ccccc1-3 ; CC1(C)c2cccc3c2-c2c1ccc1c4ccccc4n(c21)-c1cc(-c2ccc(N(c4ccc5oc6ccccc6c5c4)c4cccc5c4-c4ccccc4C5(c4ccccc4)c4ccccc4)cc2)ccc1-3]

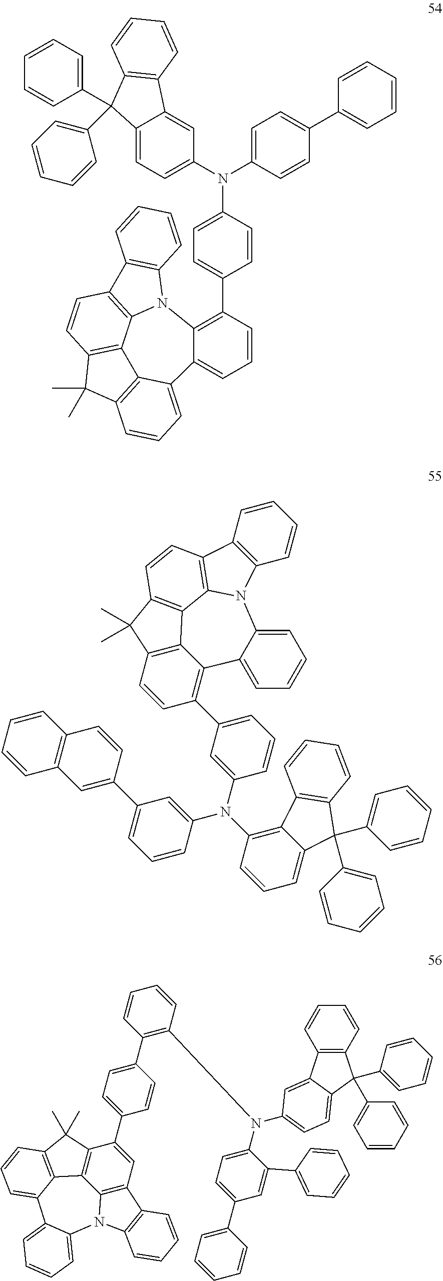 [SMILES: CC1(C)c2cccc3c2-c2c1ccc1c4ccccc4n(c21)-c1c(-c2ccc(N(c4ccc(-c5ccccc5)cc4)c4ccc5c(c4)-c4ccccc4C5(c4ccccc4)c4ccccc4)cc2)cccc1-3 ; CC1(C)c2ccc(-c3cccc(N(c4cccc(-c5ccc6ccccc6c5)c4)c4cccc5c4-c4ccccc4C5(c4ccccc4)c4ccccc4)c3)c3c2-c2c1ccc1c4ccccc4n(c21)-c1ccccc1-3 ; CC1(C)c2cccc3c2-c2c1c(-c1ccc(-c4ccccc4N(c4ccc5c(c4)-c4ccccc4C5(c4ccccc4)c4ccccc4)c4ccc(-c5ccccc5)cc4-c4ccccc4)cc1)cc1c4ccccc4n(c21)-c1ccccc1-3]

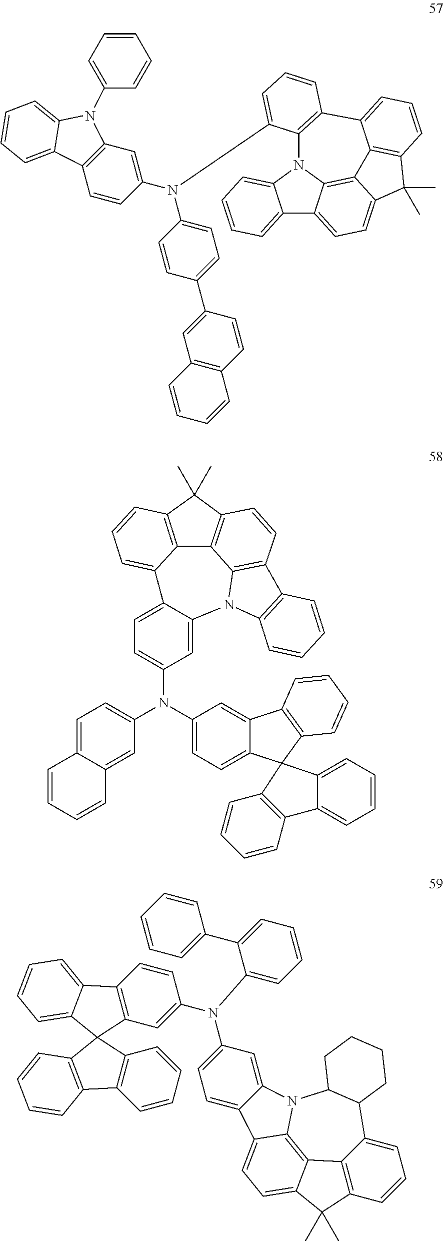

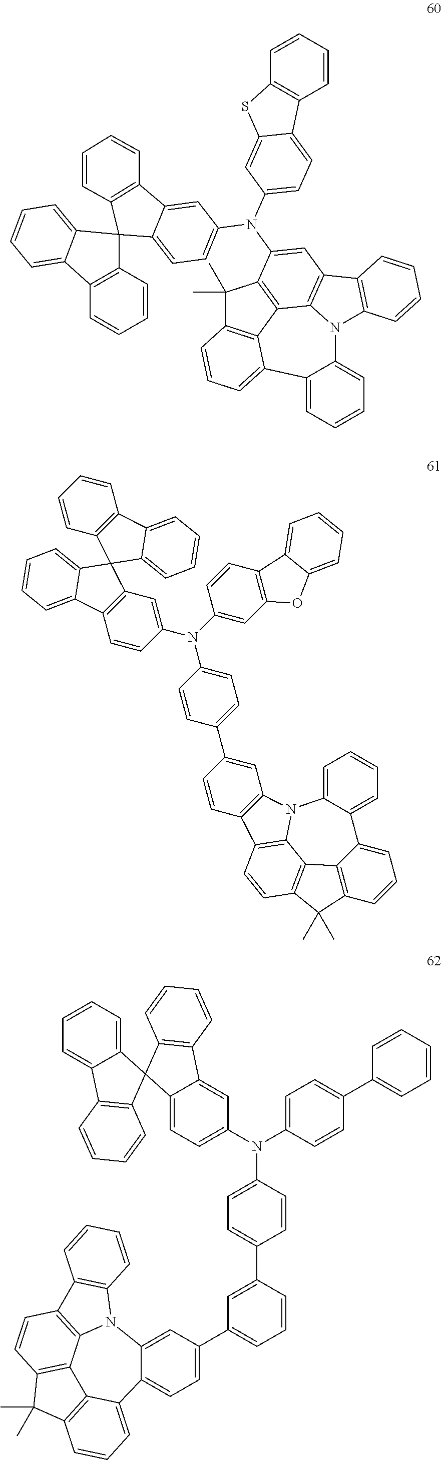 [SMILES: CC1(C)c2cccc3c2-c2c1c(N(c1ccc4c(c1)-c1ccccc1C41c4ccccc4-c4ccccc41)c1ccc4c(c1)sc1ccccc14)cc1c4ccccc4n(c21)-c1ccccc1-3 ; CC1(C)c2cccc3c2-c2c1ccc1c4ccc(-c5ccc(N(c6ccc7c(c6)C6(c8ccccc8-c8ccccc86)c6ccccc6-7)c6ccc7c(c6)oc6ccccc67)cc5)cc4n(c21)-c1ccccc1-3 ; CC1(C)c2cccc3c2-c2c1ccc1c4ccccc4n(c21)-c1cc(-c2cccc(-c4ccc(N(c5ccc(-c6ccccc6)cc5)c5ccc6c(c5)-c5ccccc5C65c6ccccc6-c6ccccc65)cc4)c2)ccc1-3]

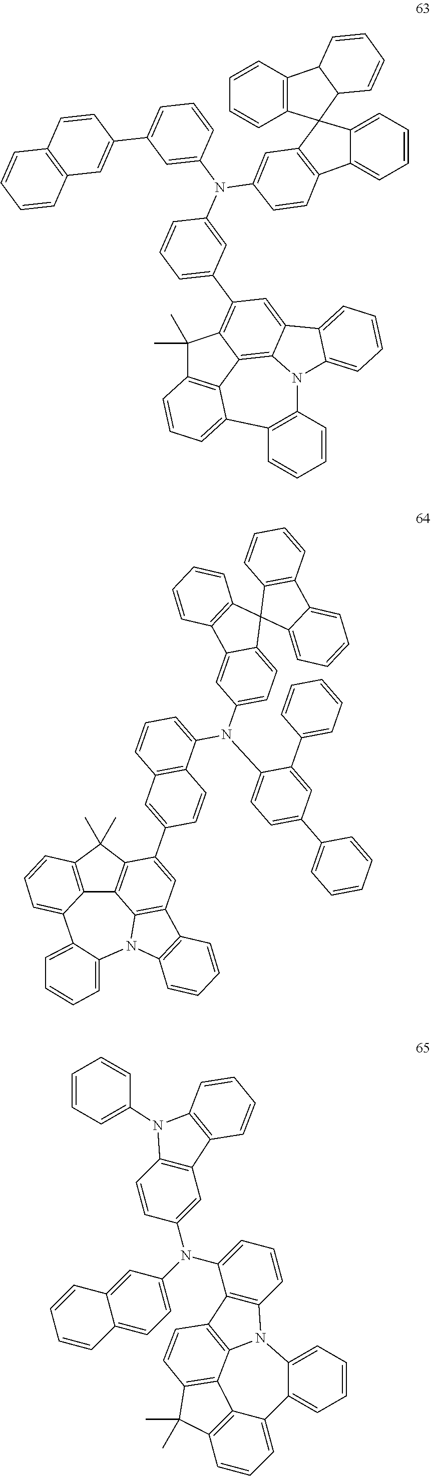 [SMILES: CC1(C)c2cccc3c2-c2c1c(-c1cccc(N(c4cccc(-c5ccc6ccccc6c5)c4)c4ccc5c(c4)C4(c6ccccc6-5)c5ccccc5C5C=CC=CC54)c1)cc1c4ccccc4n(c21)-c1ccccc1-3 ; CC1(C)c2cccc3c2-c2c1c(-c1ccc4c(N(c5ccc6c(c5)-c5ccccc5C65c6ccccc6-c6ccccc65)c5ccc(-c6ccccc6)cc5-c5ccccc5)cccc4c1)cc1c4ccccc4n(c21)-c1ccccc1-3 ; CC1(C)c2cccc3c2-c2c1ccc1c4c(N(c5ccc6ccccc6c5)c5ccc6c(c5)c5ccccc5n6-c5ccccc5)cccc4n(c21)-c1ccccc1-3]

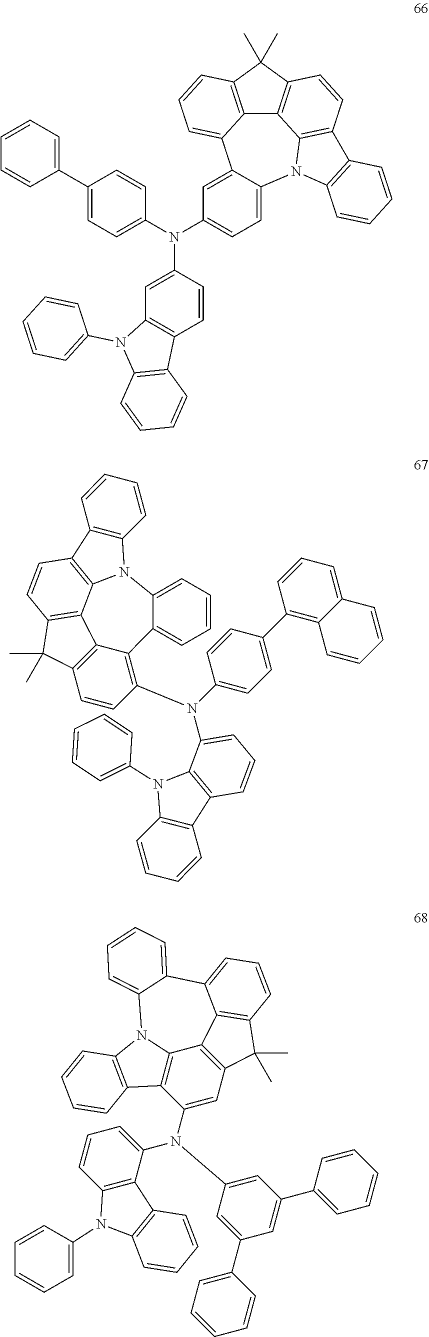 [SMILES: CC1(C)c2cccc3c2-c2c1ccc1c4ccccc4n(c21)-c1ccc(N(c2ccc(-c4ccccc4)cc2)c2ccc4c5ccccc5n(-c5ccccc5)c4c2)cc1-3 ; CC1(C)c2ccc(N(c3ccc(-c4cccc5ccccc45)cc3)c3cccc4c5ccccc5n(-c5ccccc5)c34)c3c2-c2c1ccc1c4ccccc4n(c21)-c1ccccc1-3 ; CC1(C)c2cccc3c2-c2c1cc(N(c1cc(-c4ccccc4)cc(-c4ccccc4)c1)c1cccc4c1c1ccccc1n4-c1ccccc1)c1c4ccccc4n(c21)-c1ccccc1-3]

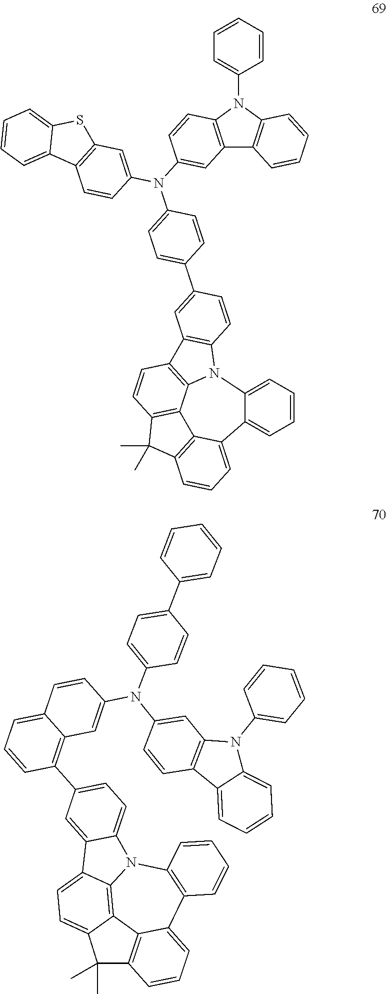

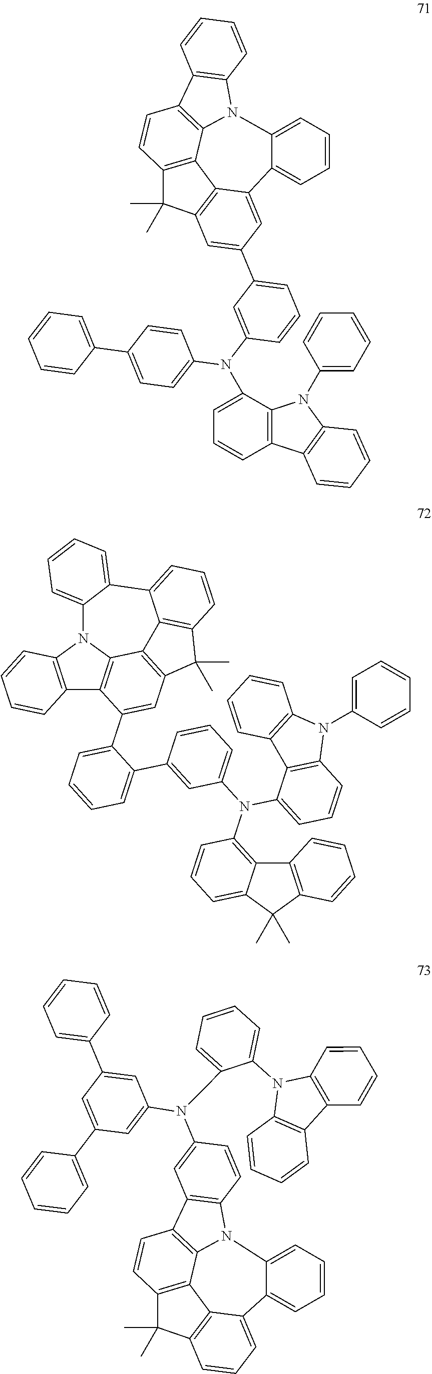 [SMILES: CC1(C)c2cc(-c3cccc(N(c4ccc(-c5ccccc5)cc4)c4cccc5c6ccccc6n(-c6ccccc6)c45)c3)cc3c2-c2c1ccc1c4ccccc4n(c21)-c1ccccc1-3 ; CC1(C)c2ccccc2-c2c(N(c3cccc(-c4ccccc4-c4cc5c6c7c4c4ccccc4n7-c4ccccc4-c4cccc(c4-6)C5(C)C)c3)c3cccc4c3c3ccccc3n4-c3ccccc3)cccc21 ; CC1(C)c2cccc3c2-c2c1ccc1c4cc(N(c5cc(-c6ccccc6)cc(-c6ccccc6)c5)c5ccccc5-n5c6ccccc6c6ccccc65)ccc4n(c21)-c1ccccc1-3]

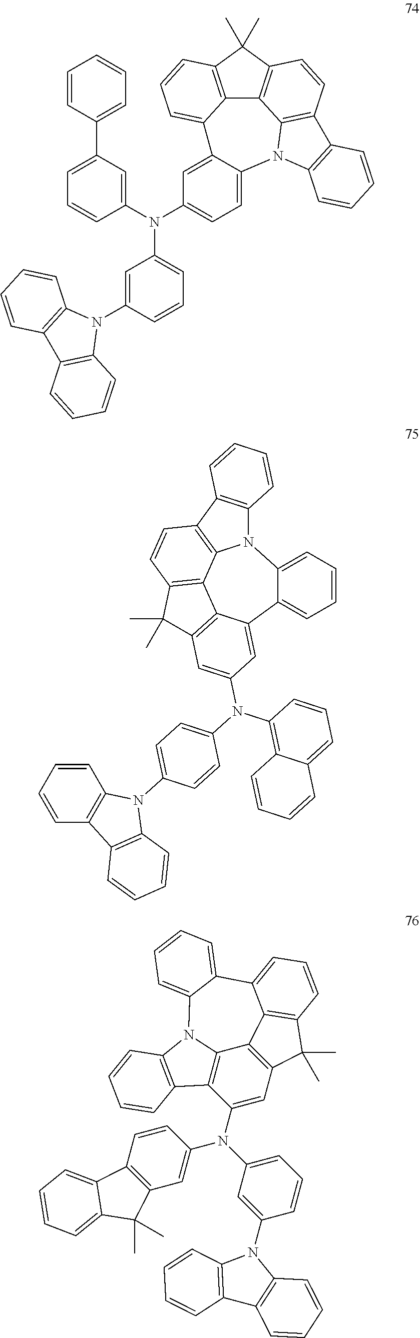 [SMILES: CC1(C)c2cccc3c2-c2c1ccc1c4ccccc4n(c21)-c1ccc(N(c2cccc(-c4ccccc4)c2)c2cccc(-n4c5ccccc5c5ccccc54)c2)cc1-3 ; CC1(C)c2cc(N(c3ccc(-n4c5ccccc5c5ccccc54)cc3)c3cccc4ccccc34)cc3c2-c2c1ccc1c4ccccc4n(c21)-c1ccccc1-3 ; CC1(C)c2ccccc2-c2ccc(N(c3cccc(-n4c5ccccc5c5ccccc54)c3)c3cc4c5c6c3c3ccccc3n6-c3ccccc3-c3cccc(c3-5)C4(C)C)cc21]

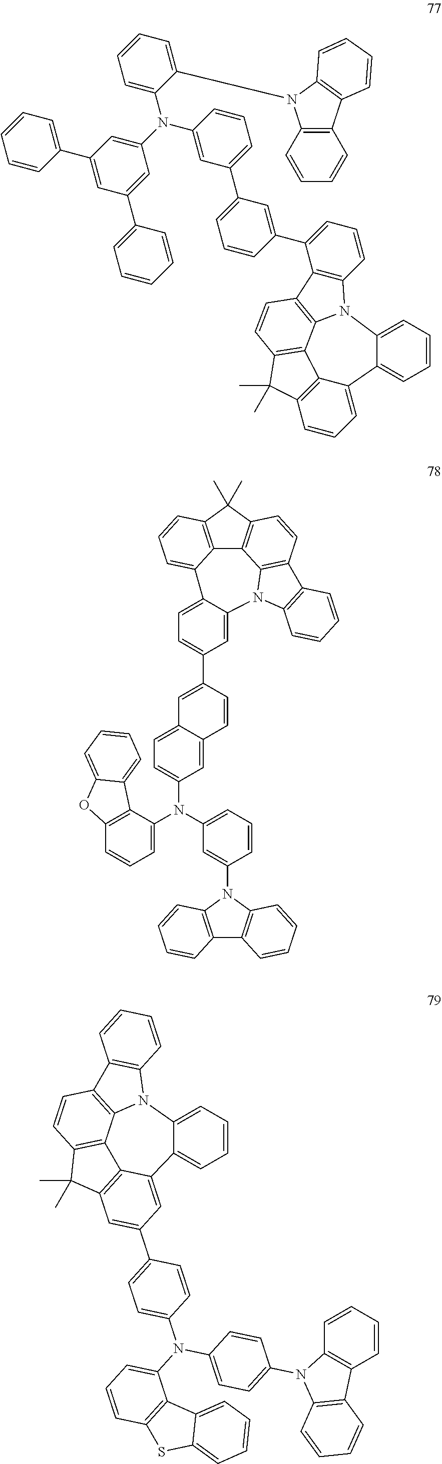 [SMILES: CC1(C)c2cccc3c2-c2c1ccc1c4c(-c5cccc(-c6cccc(N(c7cc(-c8ccccc8)cc(-c8ccccc8)c7)c7ccccc7-n7c8ccccc8c8ccccc87)c6)c5)cccc4n(c21)-c1ccccc1-3 ; CC1(C)c2cccc3c2-c2c1ccc1c4ccccc4n(c21)-c1cc(-c2ccc4cc(N(c5cccc(-n6c7ccccc7c7ccccc76)c5)c5cccc6oc7ccccc7c56)ccc4c2)ccc1-3 ; CC1(C)c2cc(-c3ccc(N(c4ccc(-n5c6ccccc6c6ccccc65)cc4)c4cccc5sc6ccccc6c45)cc3)cc3c2-c2c1ccc1c4ccccc4n(c21)-c1ccccc1-3]

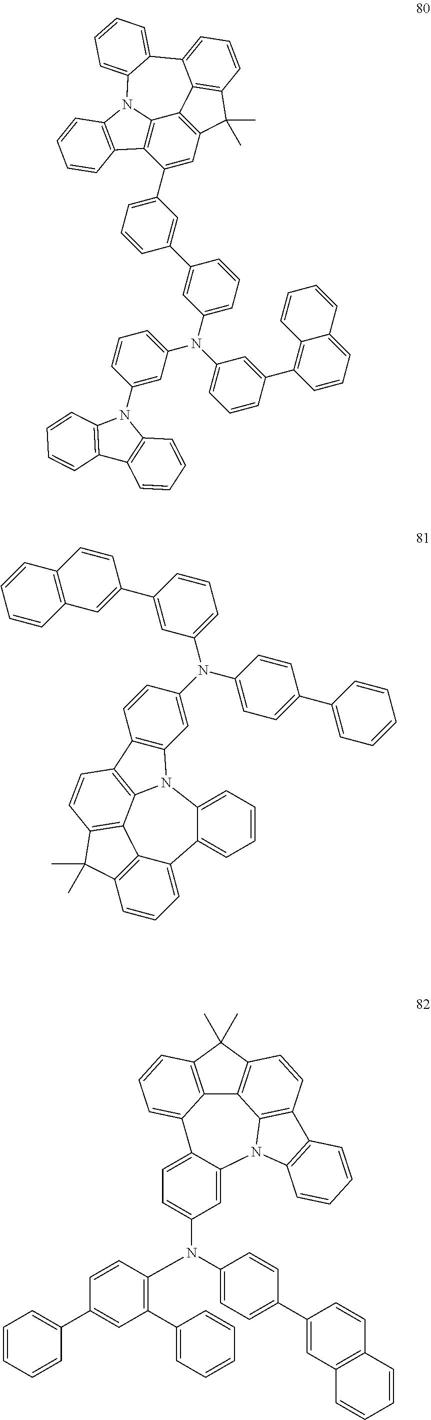 [SMILES: CC1(C)c2cccc3c2-c2c1cc(-c1cccc(-c4cccc(N(c5cccc(-c6cccc7ccccc67)c5)c5cccc(-n6c7ccccc7c7ccccc76)c5)c4)c1)c1c4ccccc4n(c21)-c1ccccc1-3 ; CC1(C)c2cccc3c2-c2c1ccc1c4ccc(N(c5ccc(-c6ccccc6)cc5)c5cccc(-c6ccc7ccccc7c6)c5)cc4n(c21)-c1ccccc1-3 ; CC1(C)c2cccc3c2-c2c1ccc1c4ccccc4n(c21)-c1cc(N(c2ccc(-c4ccc5ccccc5c4)cc2)c2ccc(-c4ccccc4)cc2-c2ccccc2)ccc1-3]

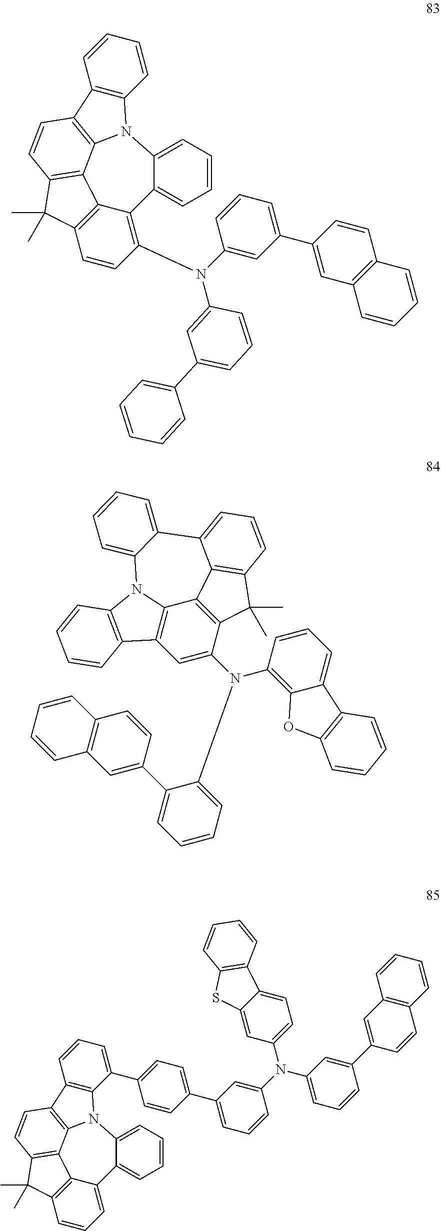 [SMILES: CC1(C)c2ccc(N(c3cccc(-c4ccccc4)c3)c3cccc(-c4ccc5ccccc5c4)c3)c3c2-c2c1ccc1c4ccccc4n(c21)-c1ccccc1-3 ; CC1(C)c2cccc3c2-c2c1c(N(c1ccccc1-c1ccc4ccccc4c1)c1cccc4c1oc1ccccc14)cc1c4ccccc4n(c21)-c1ccccc1-3 ; CC1(C)c2cccc3c2-c2c1ccc1c4cccc(-c5ccc(-c6cccc(N(c7cccc(-c8ccc9ccccc9c8)c7)c7ccc8c(c7)sc7ccccc78)c6)cc5)c4n(c21)-c1ccccc1-3]

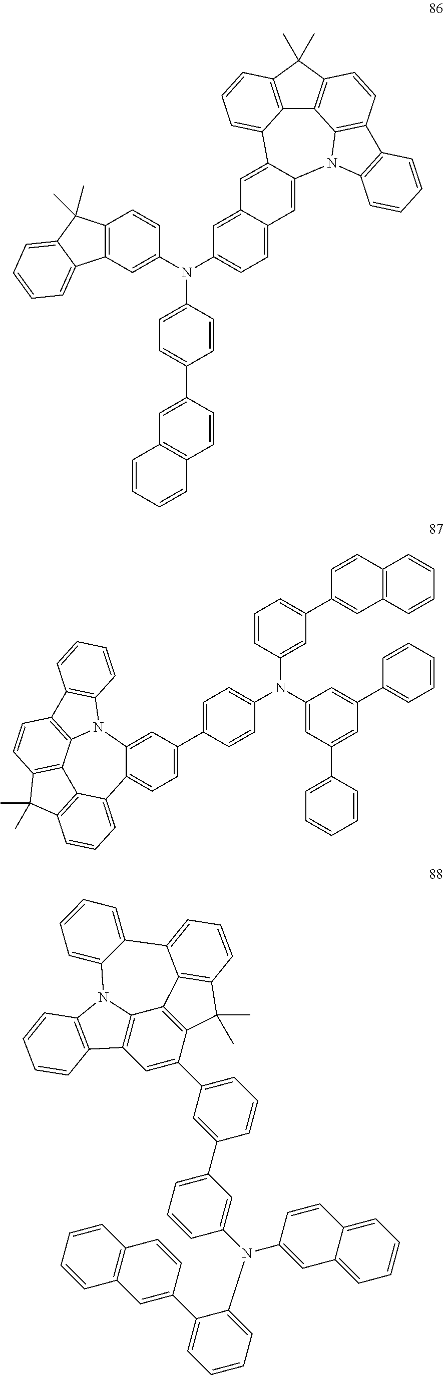 [SMILES: CC1(C)c2ccccc2-c2cc(N(c3ccc(-c4ccc5ccccc5c4)cc3)c3ccc4cc5c(cc4c3)-c3cccc4c3-c3c(ccc6c7ccccc7n-5c36)C4(C)C)ccc21 ; CC1(C)c2cccc3c2-c2c1ccc1c4ccccc4n(c21)-c1cc(-c2ccc(N(c4cccc(-c5ccc6ccccc6c5)c4)c4cc(-c5ccccc5)cc(-c5ccccc5)c4)cc2)ccc1-3 ; CC1(C)c2cccc3c2-c2c1c(-c1cccc(-c4cccc(N(c5ccc6ccccc6c5)c5ccccc5-c5ccc6ccccc6c5)c4)c1)cc1c4ccccc4n(c21)-c1ccccc1-3]

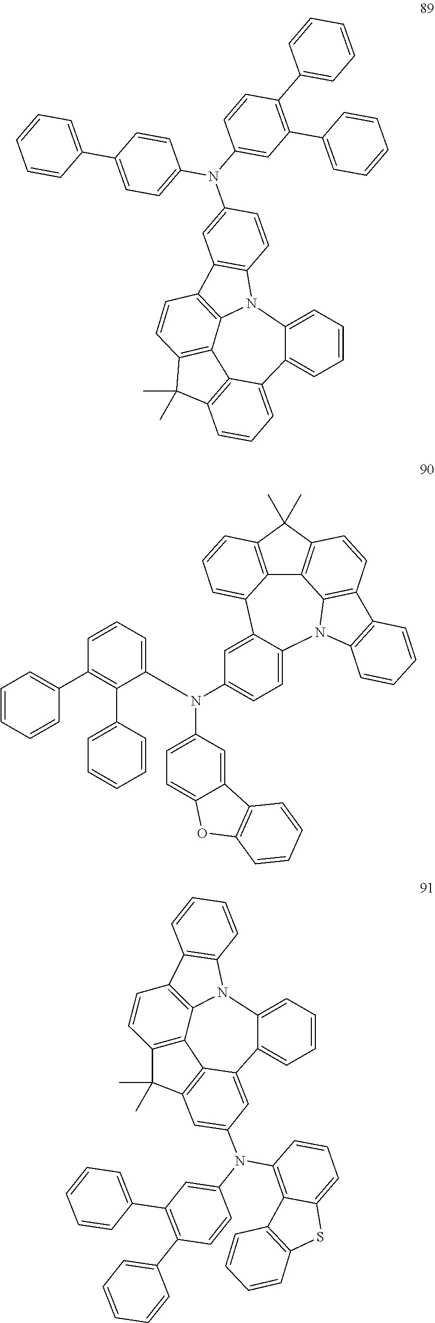 [SMILES: CC1(C)c2cccc3c2-c2c1ccc1c4cc(N(c5ccc(-c6ccccc6)cc5)c5ccc(-c6ccccc6)c(-c6ccccc6)c5)ccc4n(c21)-c1ccccc1-3 ; CC1(C)c2cccc3c2-c2c1ccc1c4ccccc4n(c21)-c1ccc(N(c2ccc4oc5ccccc5c4c2)c2cccc(-c4ccccc4)c2-c2ccccc2)cc1-3 ; CC1(C)c2cc(N(c3ccc(-c4ccccc4)c(-c4ccccc4)c3)c3cccc4sc5ccccc5c34)cc3c2-c2c1ccc1c4ccccc4n(c21)-c1ccccc1-3]

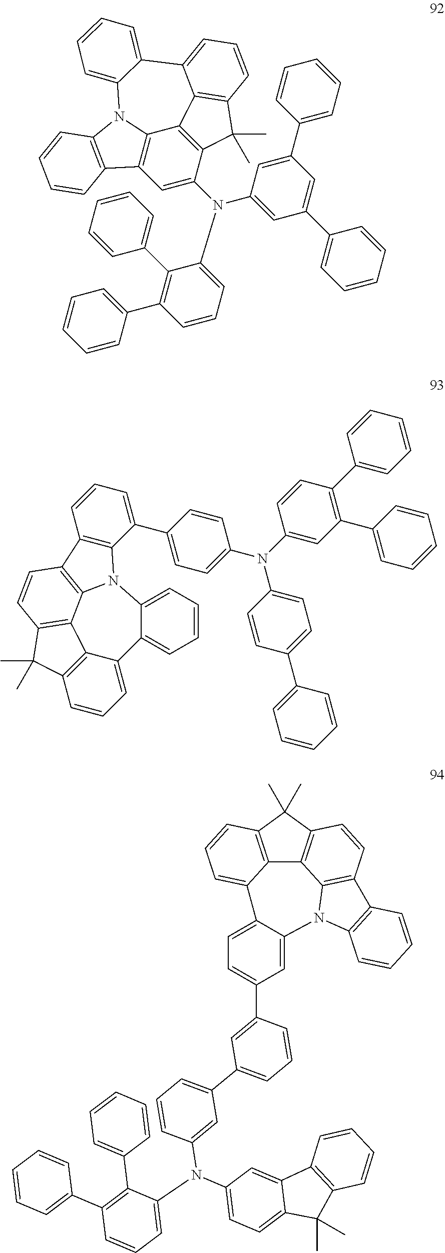

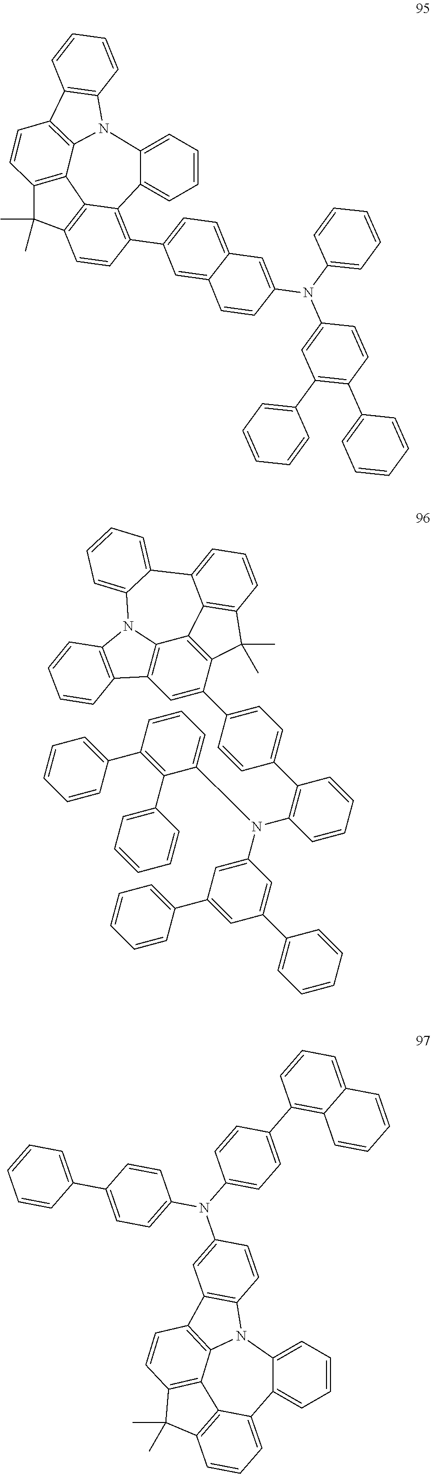 [SMILES: CC1(C)c2ccc(-c3ccc4cc(N(c5ccccc5)c5ccc(-c6ccccc6)c(-c6ccccc6)c5)ccc4c3)c3c2-c2c1ccc1c4ccccc4n(c21)-c1ccccc1-3 ; CC1(C)c2cccc3c2-c2c1c(-c1ccc(-c4ccccc4N(c4cc(-c5ccccc5)cc(-c5ccccc5)c4)c4cccc(-c5ccccc5)c4-c4ccccc4)cc1)cc1c4ccccc4n(c21)-c1ccccc1-3 ; CC1(C)c2cccc3c2-c2c1ccc1c4cc(N(c5ccc(-c6ccccc6)cc5)c5ccc(-c6cccc7ccccc67)cc5)ccc4n(c21)-c1ccccc1-3]

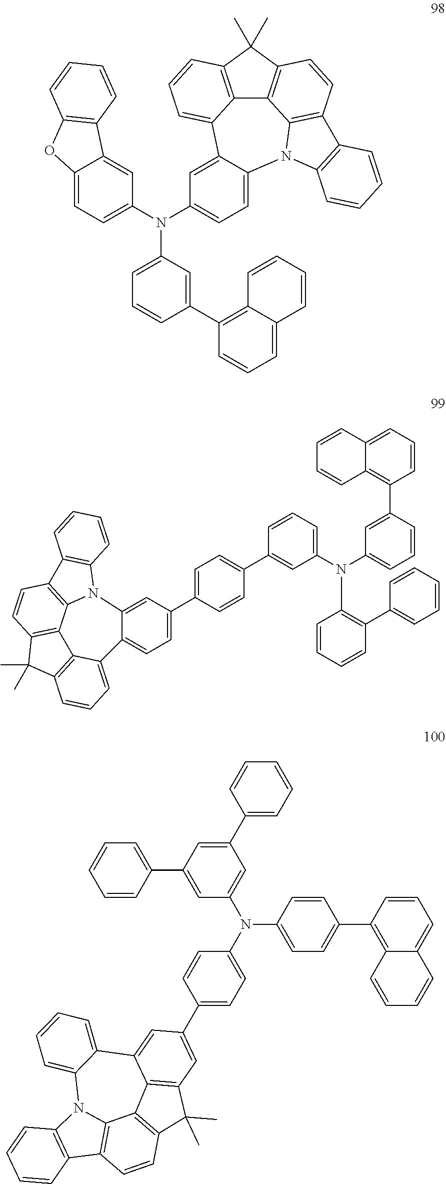 [SMILES: CC1(C)c2cccc3c2-c2c1ccc1c4ccccc4n(c21)-c1ccc(N(c2cccc(-c4cccc5ccccc45)c2)c2ccc4oc5ccccc5c4c2)cc1-3 ; CC1(C)c2cccc3c2-c2c1ccc1c4ccccc4n(c21)-c1cc(-c2ccc(-c4cccc(N(c5cccc(-c6cccc7ccccc67)c5)c5ccccc5-c5ccccc5)c4)cc2)ccc1-3 ; CC1(C)c2cc(-c3ccc(N(c4ccc(-c5cccc6ccccc56)cc4)c4cc(-c5ccccc5)cc(-c5ccccc5)c4)cc3)cc3c2-c2c1ccc1c4ccccc4n(c21)-c1ccccc1-3]

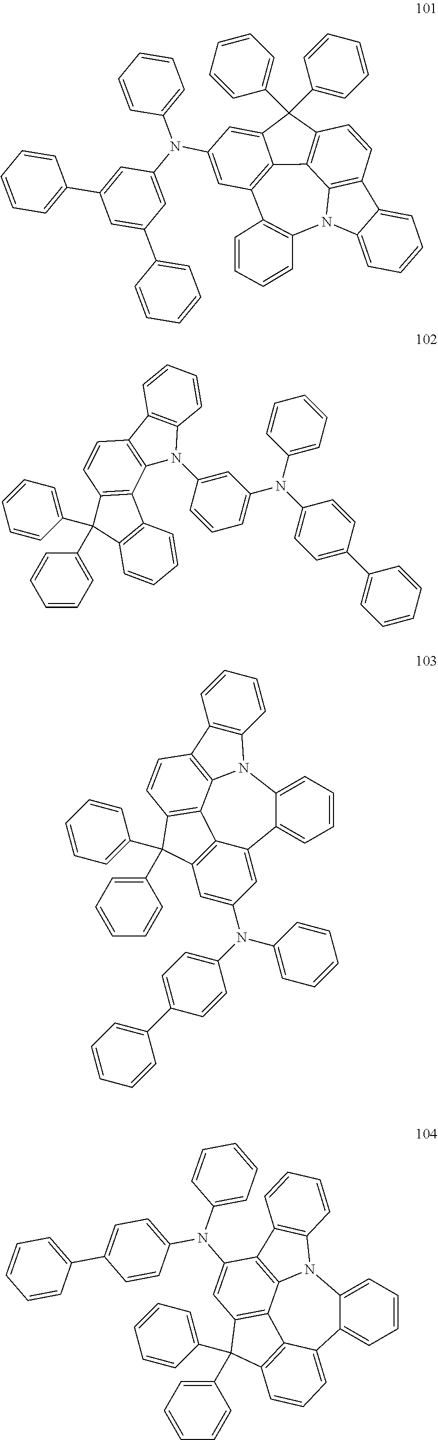

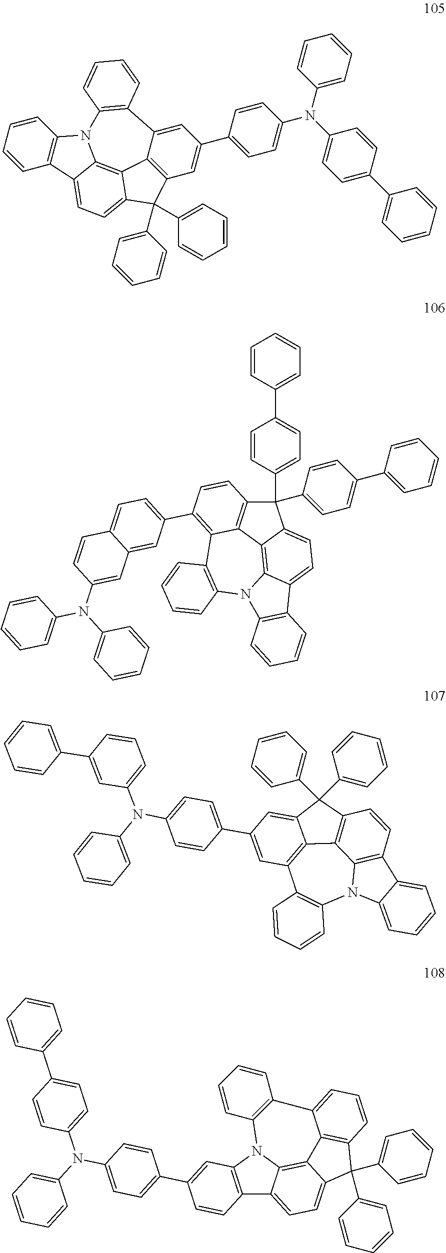 [SMILES: c1ccc(-c2ccc(N(c3ccccc3)c3ccc(-c4cc5c6c(c4)C(c4ccccc4)(c4ccccc4)c4ccc7c8ccccc8n(c7c4-6)-c4ccccc4-5)cc3)cc2)cc1 ; c1ccc(-c2ccc(C3(c4ccc(-c5ccccc5)cc4)c4ccc(-c5ccc6ccc(N(c7ccccc7)c7ccccc7)cc6c5)c5c4-c4c3ccc3c6ccccc6n(c43)-c3ccccc3-5)cc2)cc1 ; c1ccc(-c2cccc(N(c3ccccc3)c3ccc(-c4cc5c6c(c4)C(c4ccccc4)(c4ccccc4)c4ccc7c8ccccc8n(c7c4-6)-c4ccccc4-5)cc3)c2)cc1 ; c1ccc(-c2ccc(N(c3ccccc3)c3ccc(-c4ccc5c6ccc7c8c6n(c5c4)-c4ccccc4-c4cccc(c4-8)C7(c4ccccc4)c4ccccc4)cc3)cc2)cc1]

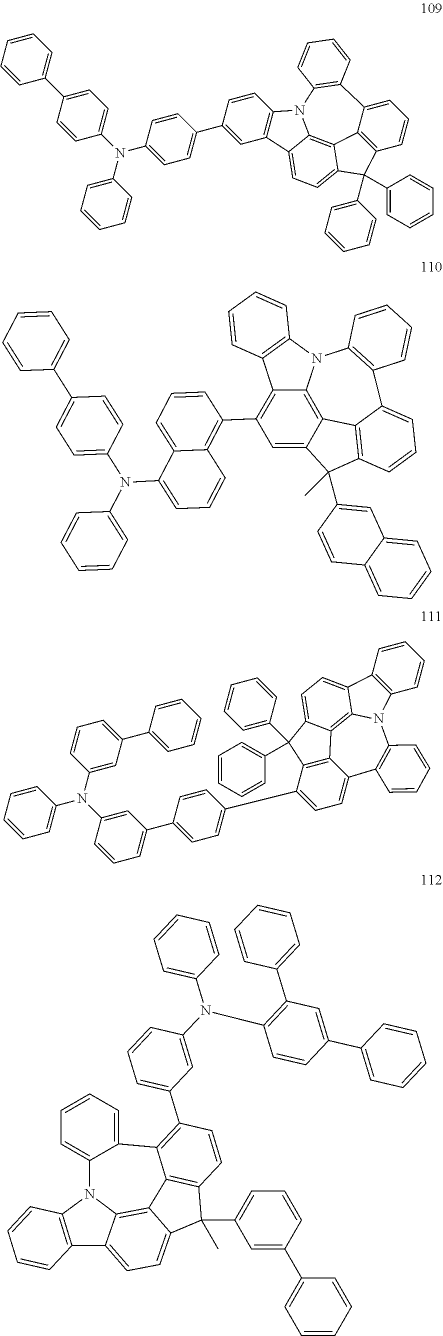

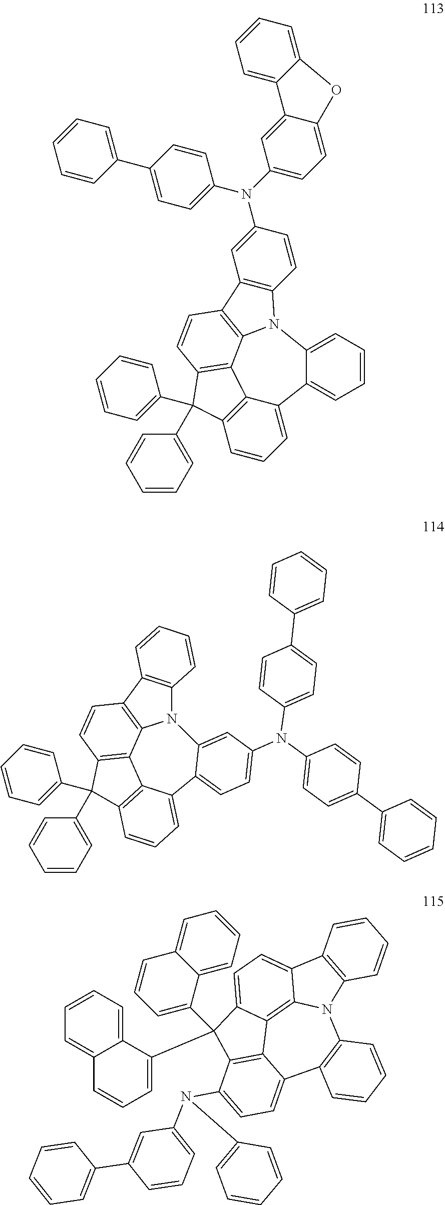 [SMILES: c1ccc(-c2ccc(N(c3ccc4oc5ccccc5c4c3)c3ccc4c(c3)c3ccc5c6c3n4-c3ccccc3-c3cccc(c3-6)C5(c3ccccc3)c3ccccc3)cc2)cc1 ; c1ccc(-c2ccc(N(c3ccc(-c4ccccc4)cc3)c3ccc4c(c3)-n3c5ccccc5c5ccc6c(c53)-c3c-4cccc3C6(c3ccccc3)c3ccccc3)cc2)cc1 ; c1ccc(-c2cccc(N(c3ccccc3)c3ccc4c5c3C(c3cccc6ccccc36)(c3cccc6ccccc36)c3ccc6c7ccccc7n(c6c3-5)-c3ccccc3-4)c2)cc1]

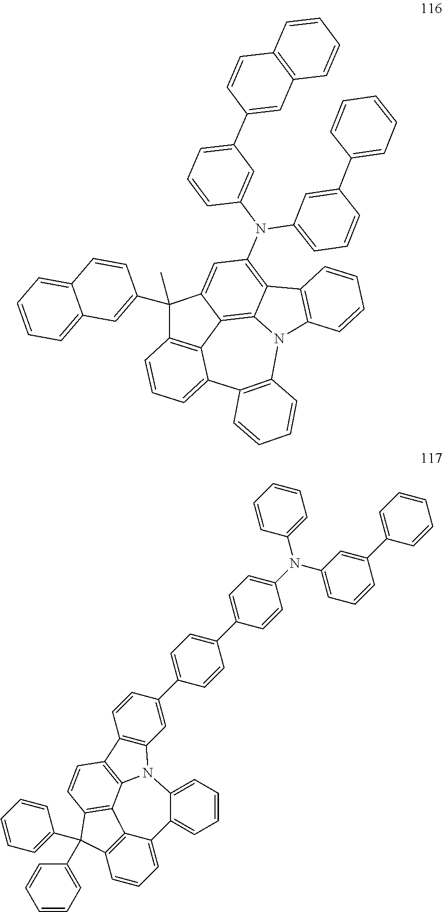 [SMILES: CC1(c2ccc3ccccc3c2)c2cccc3c2-c2c1cc(N(c1cccc(-c4ccccc4)c1)c1cccc(-c4ccc5ccccc5c4)c1)c1c4ccccc4n(c21)-c1ccccc1-3 ; c1ccc(-c2cccc(N(c3ccccc3)c3ccc(-c4ccc(-c5ccc6c7ccc8c9c7n(c6c5)-c5ccccc5-c5cccc(c5-9)C8(c5ccccc5)c5ccccc5)cc4)cc3)c2)cc1]

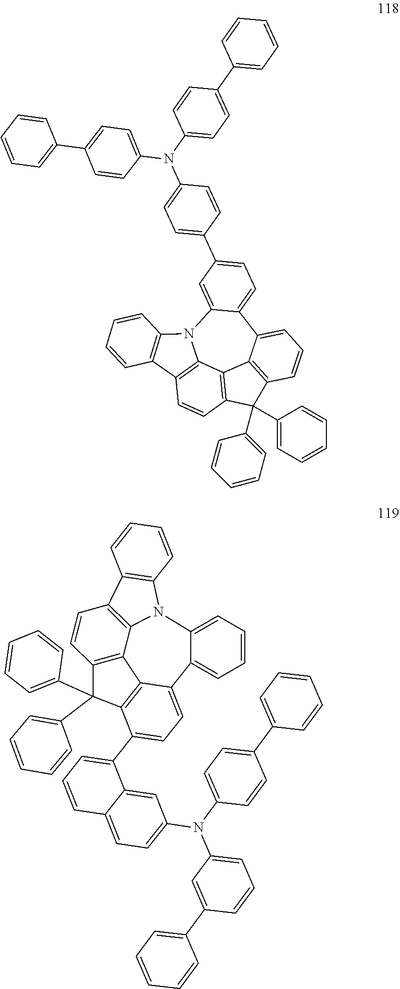 [SMILES: c1ccc(-c2ccc(N(c3ccc(-c4ccccc4)cc3)c3ccc(-c4ccc5c(c4)-n4c6ccccc6c6ccc7c(c64)-c4c-5cccc4C7(c4ccccc4)c4ccccc4)cc3)cc2)cc1 ; c1ccc(-c2ccc(N(c3cccc(-c4ccccc4)c3)c3ccc4cccc(-c5ccc6c7c5C(c5ccccc5)(c5ccccc5)c5ccc8c9ccccc9n(c8c5-7)-c5ccccc5-6)c4c3)cc2)cc1]

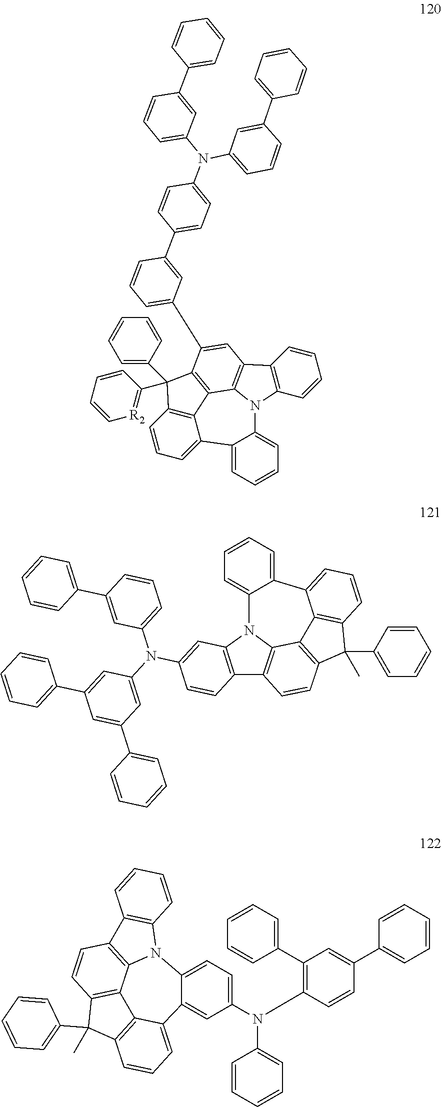

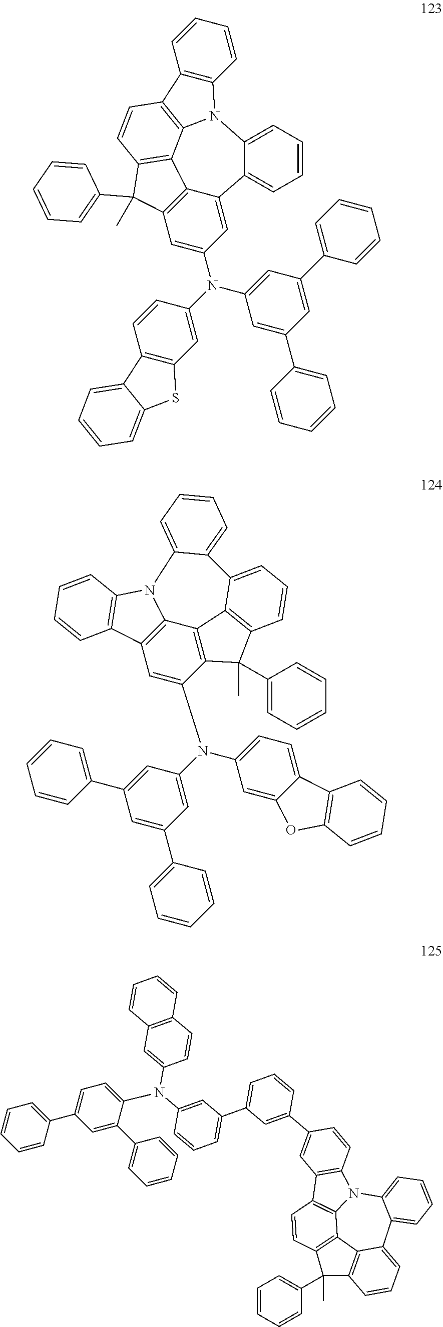 [SMILES: CC1(c2ccccc2)c2cc(N(c3cc(-c4ccccc4)cc(-c4ccccc4)c3)c3ccc4c(c3)sc3ccccc34)cc3c2-c2c1ccc1c4ccccc4n(c21)-c1ccccc1-3 ; CC1(c2ccccc2)c2cccc3c2-c2c1c(N(c1cc(-c4ccccc4)cc(-c4ccccc4)c1)c1ccc4c(c1)oc1ccccc14)cc1c4ccccc4n(c21)-c1ccccc1-3 ; CC1(c2ccccc2)c2cccc3c2-c2c1ccc1c4cc(-c5cccc(-c6cccc(N(c7ccc8ccccc8c7)c7ccc(-c8ccccc8)cc7-c7ccccc7)c6)c5)ccc4n(c21)-c1ccccc1-3]

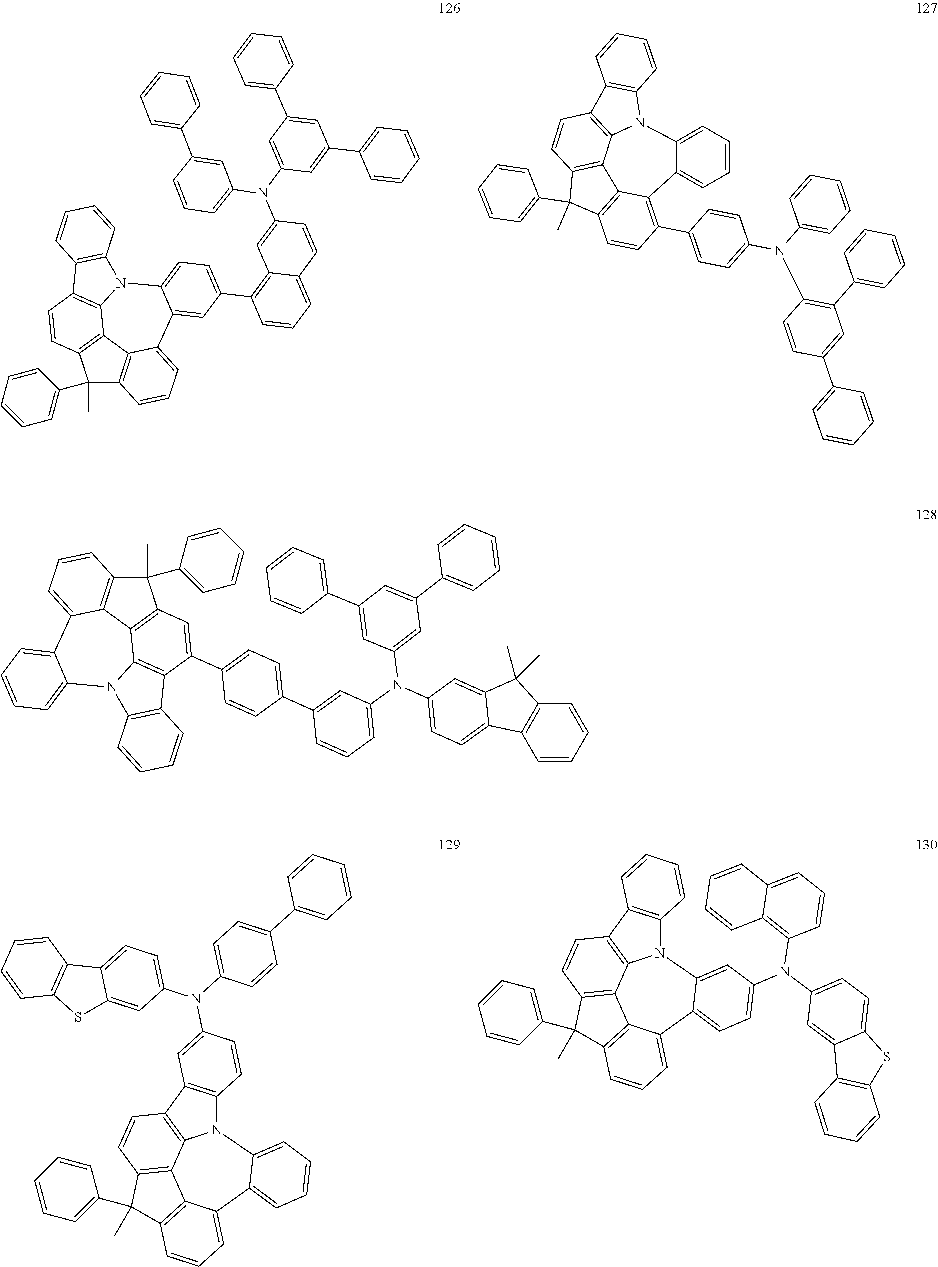

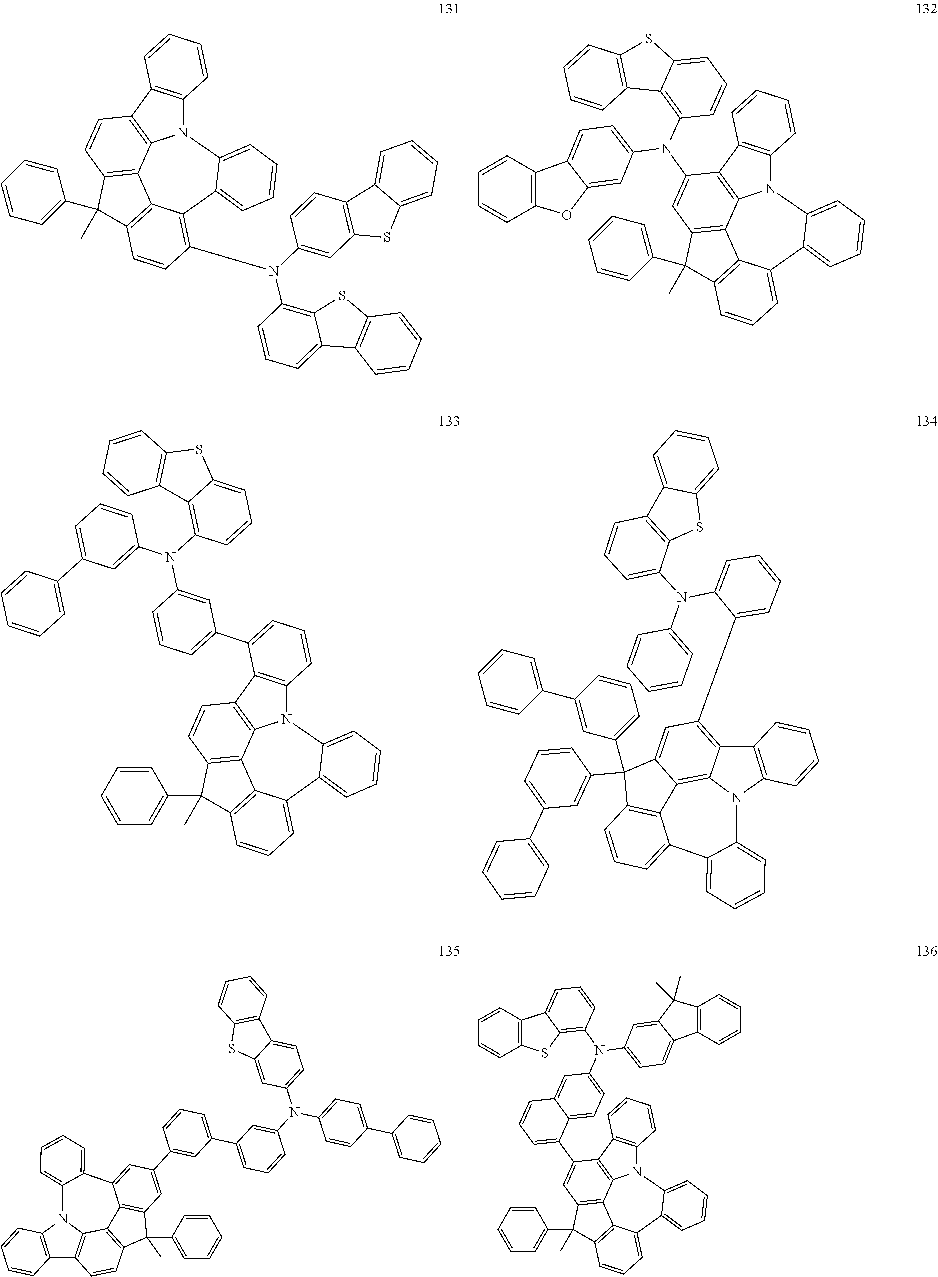

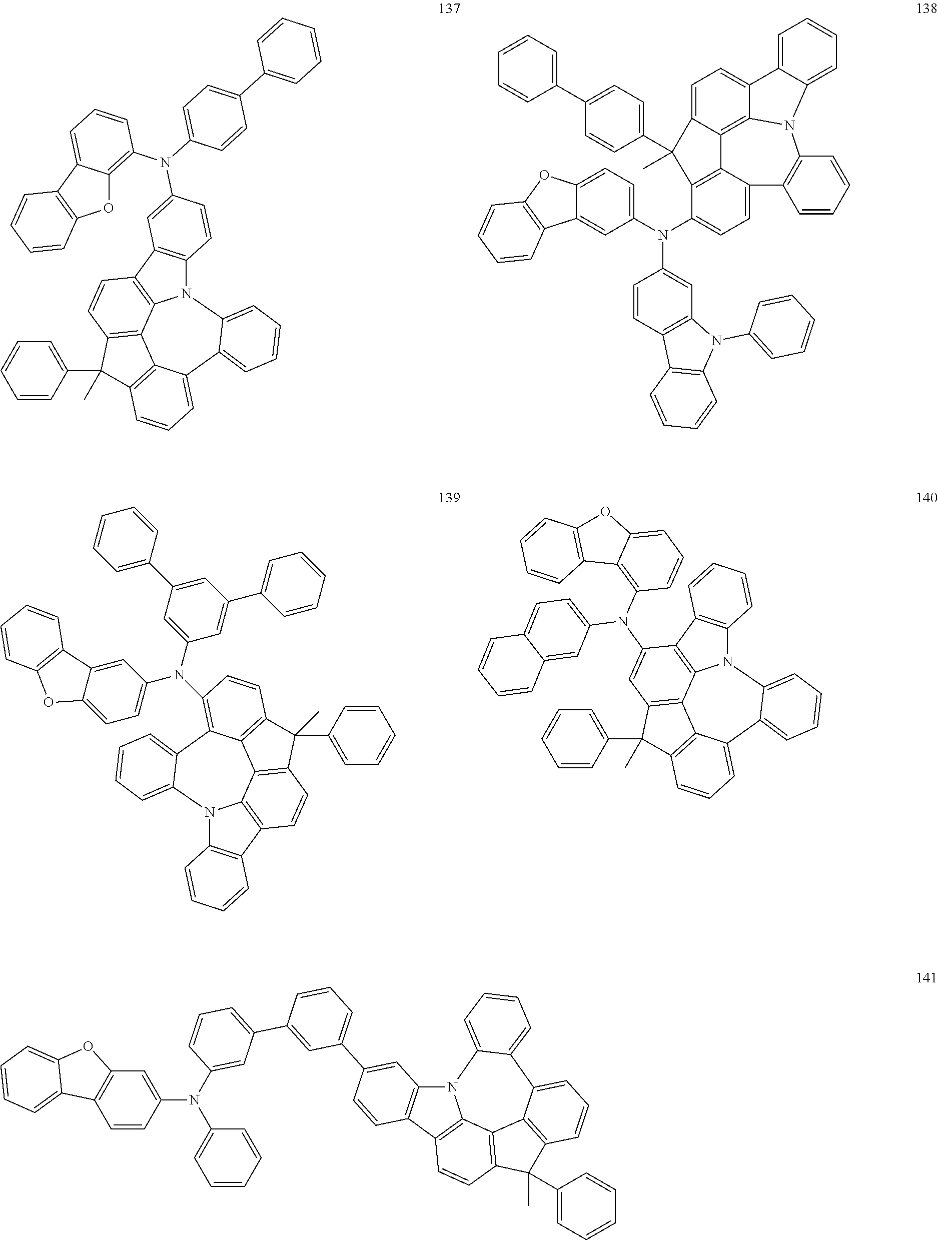

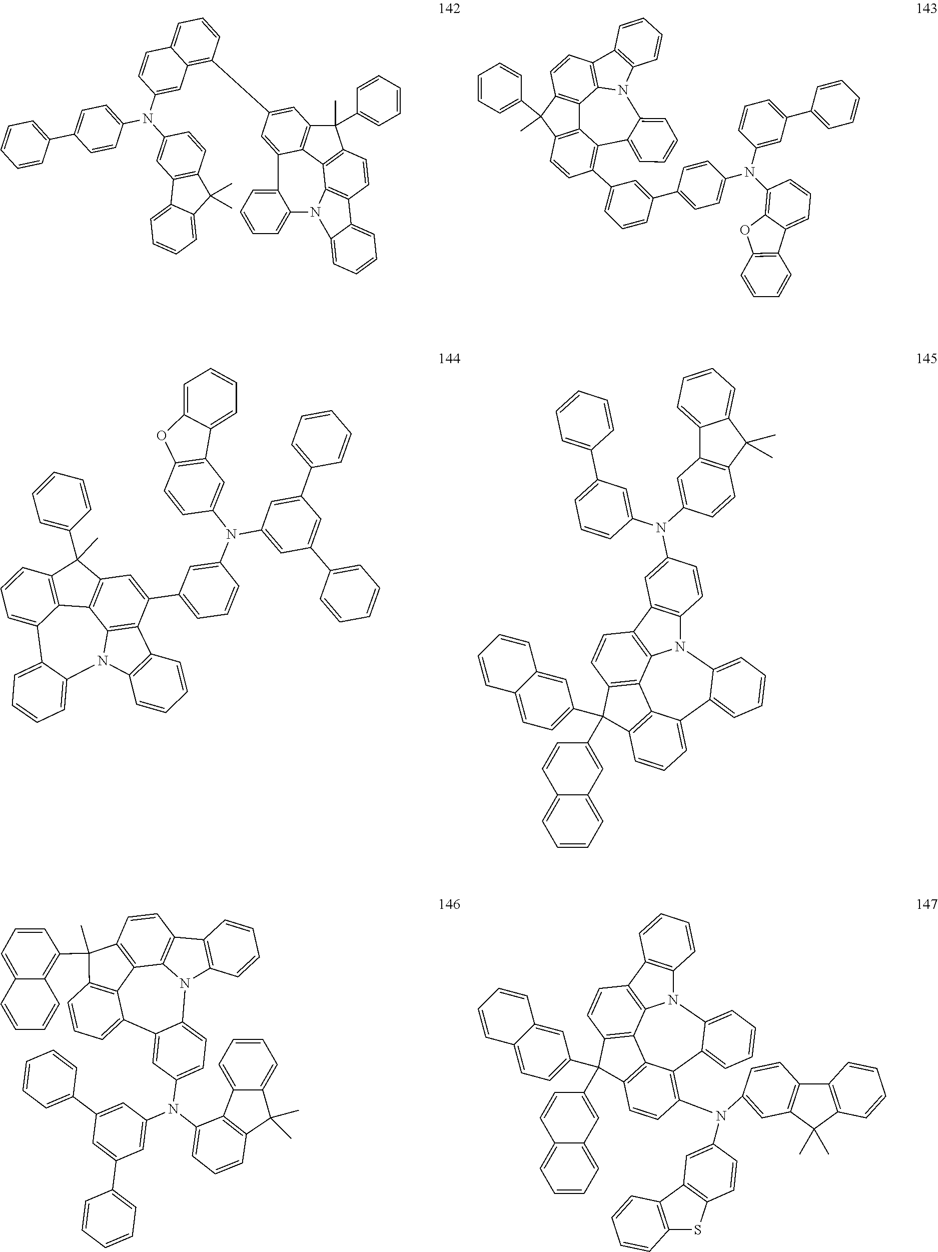

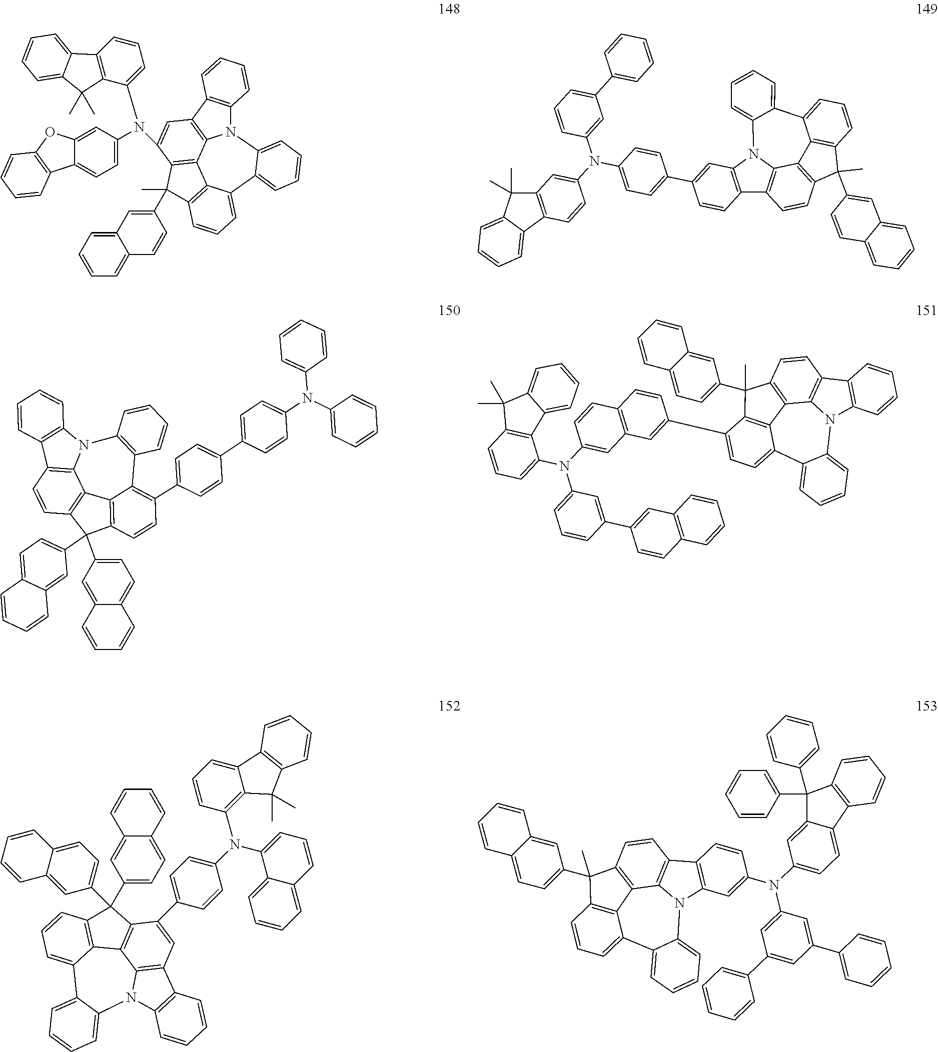

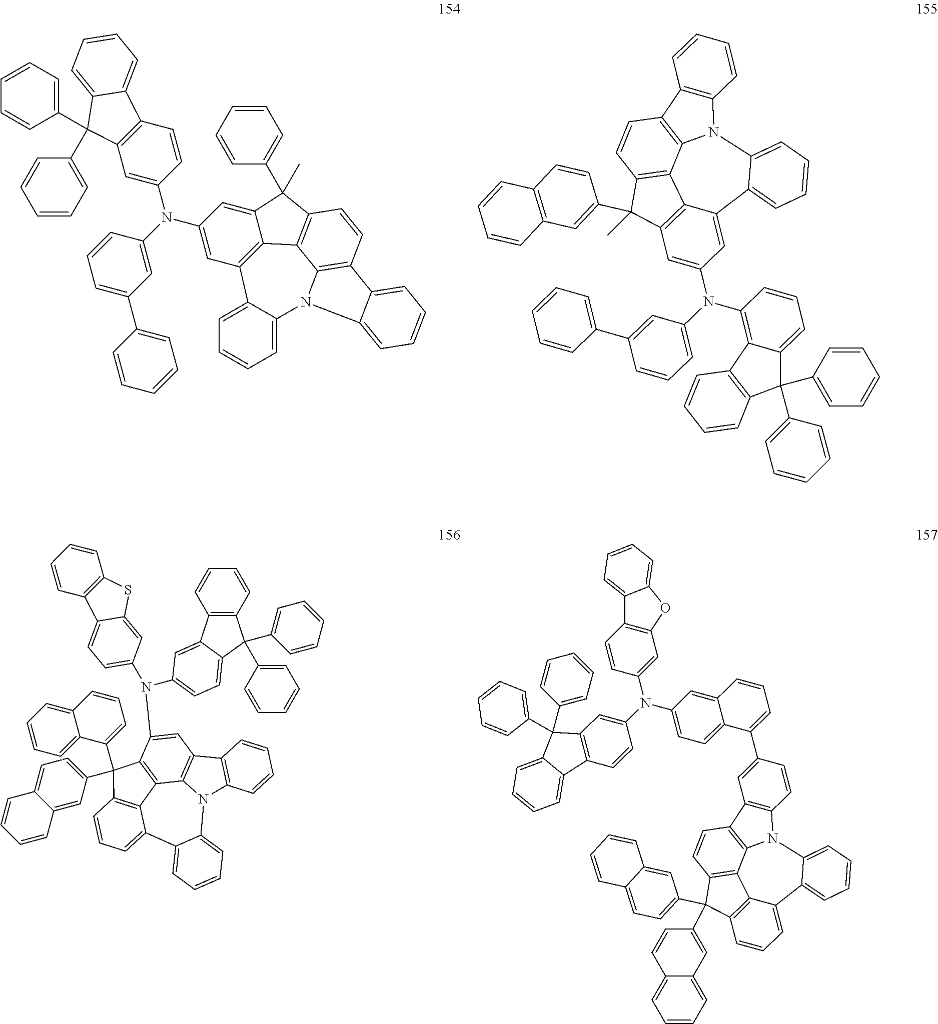

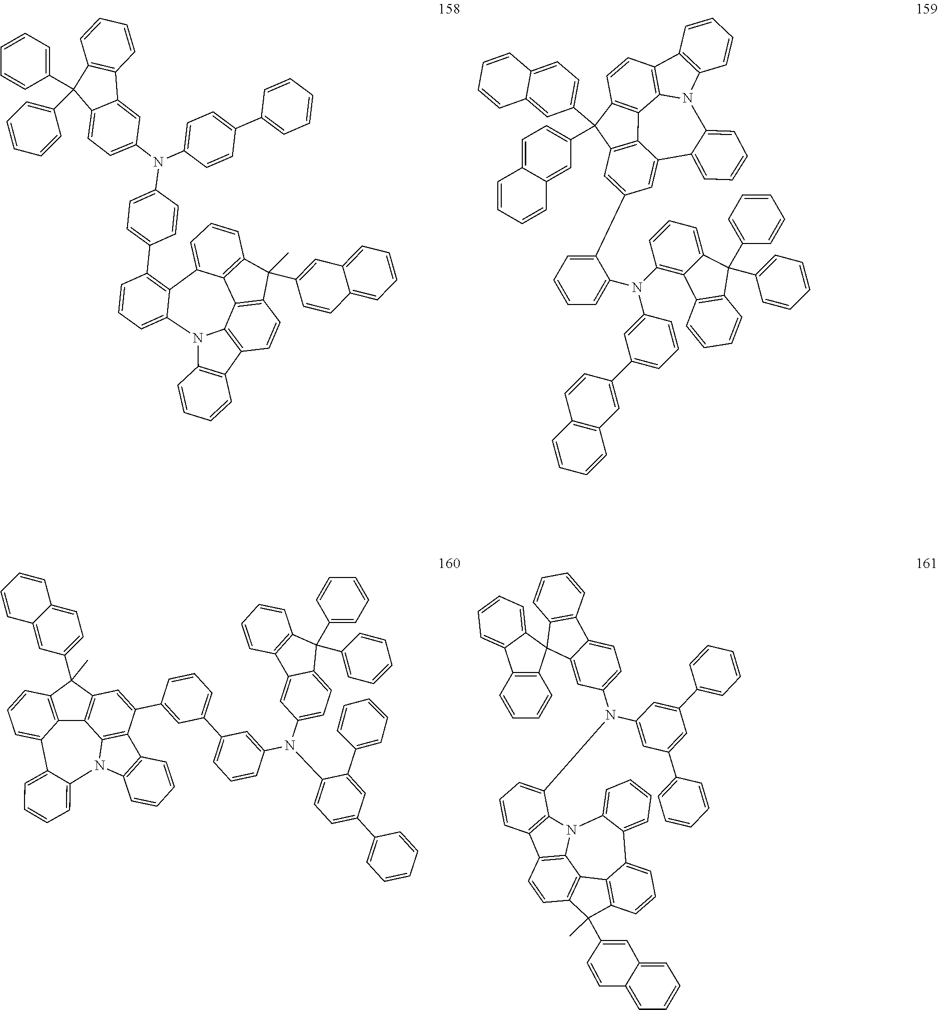

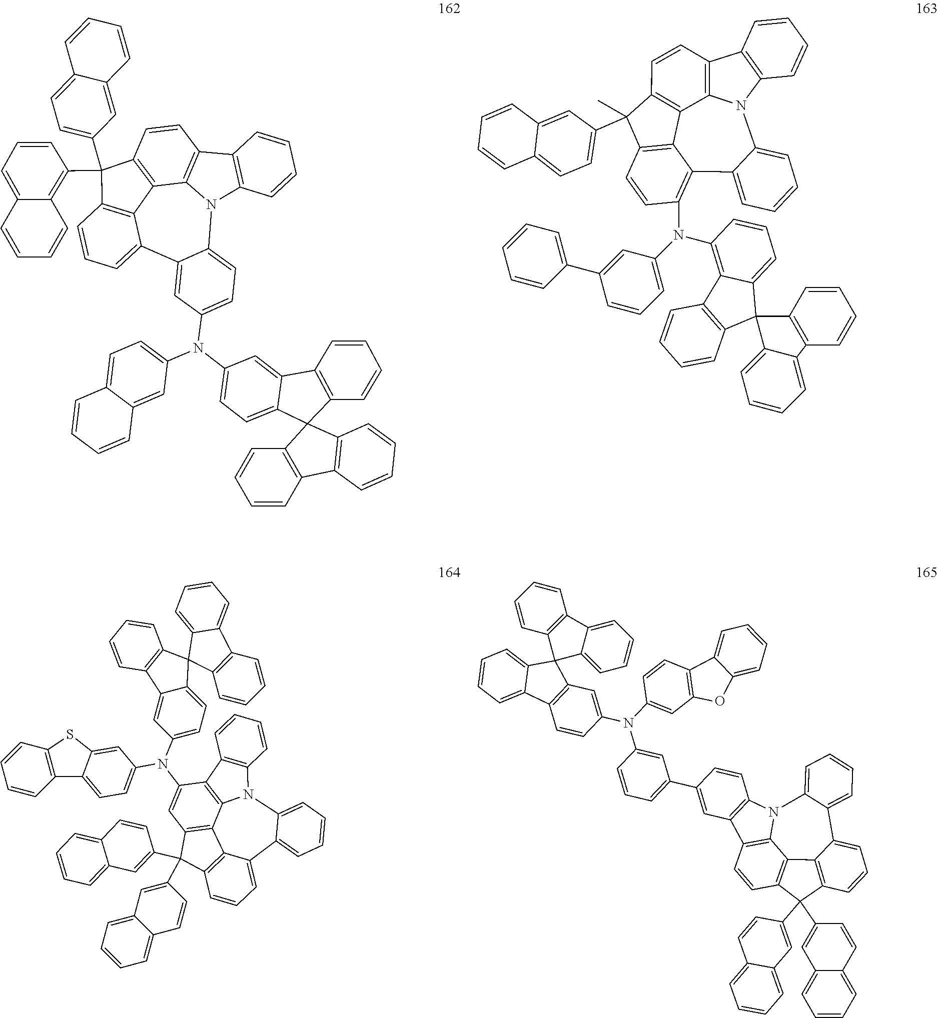

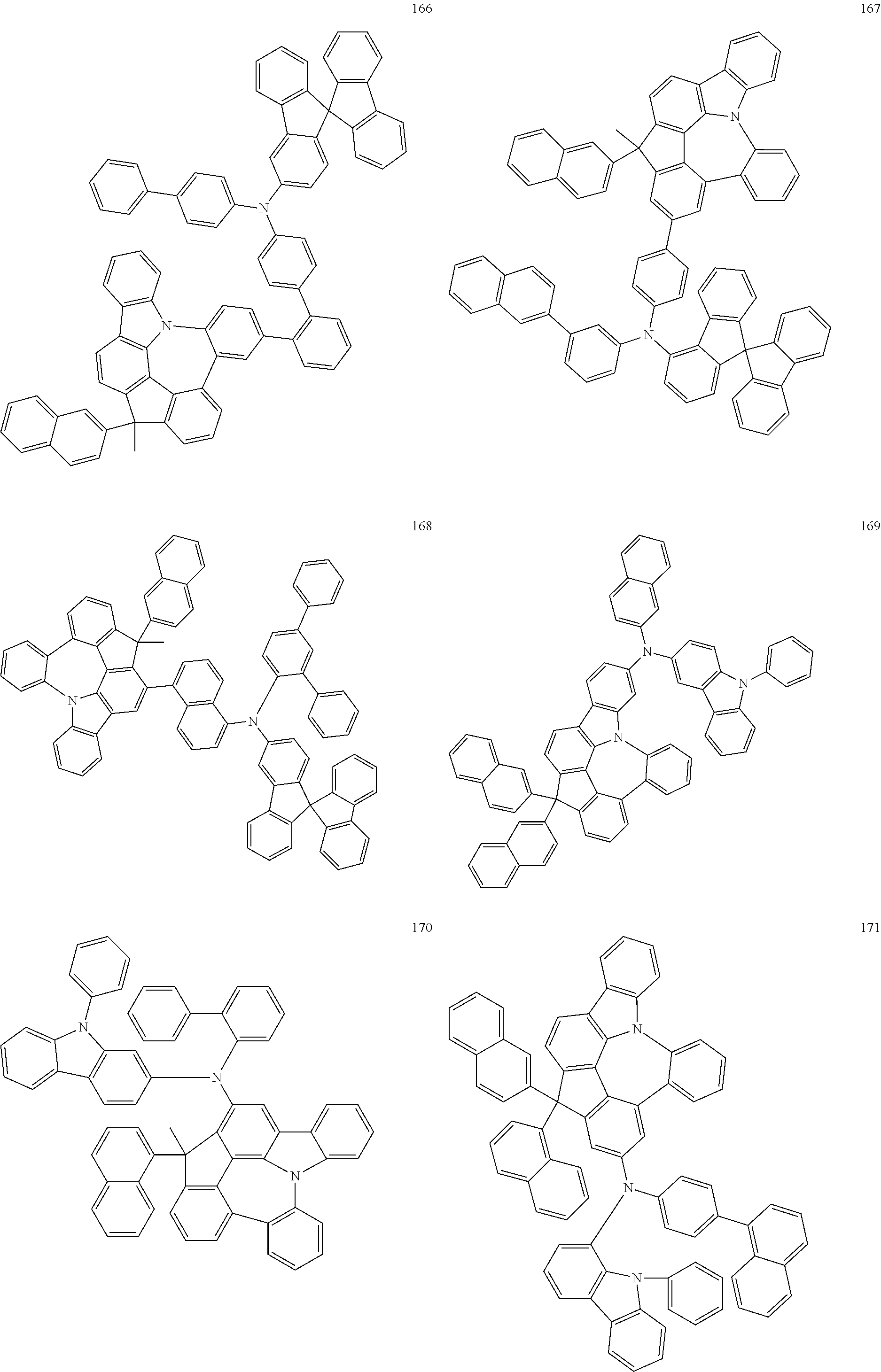

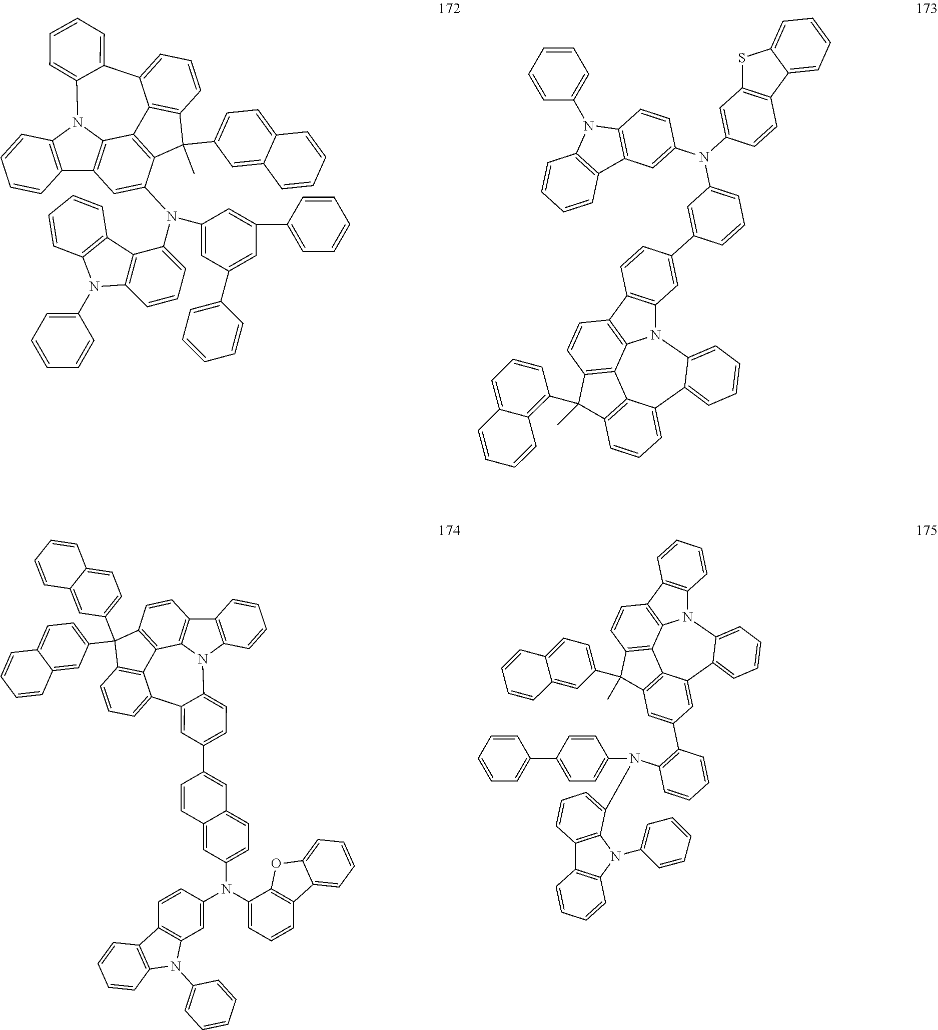

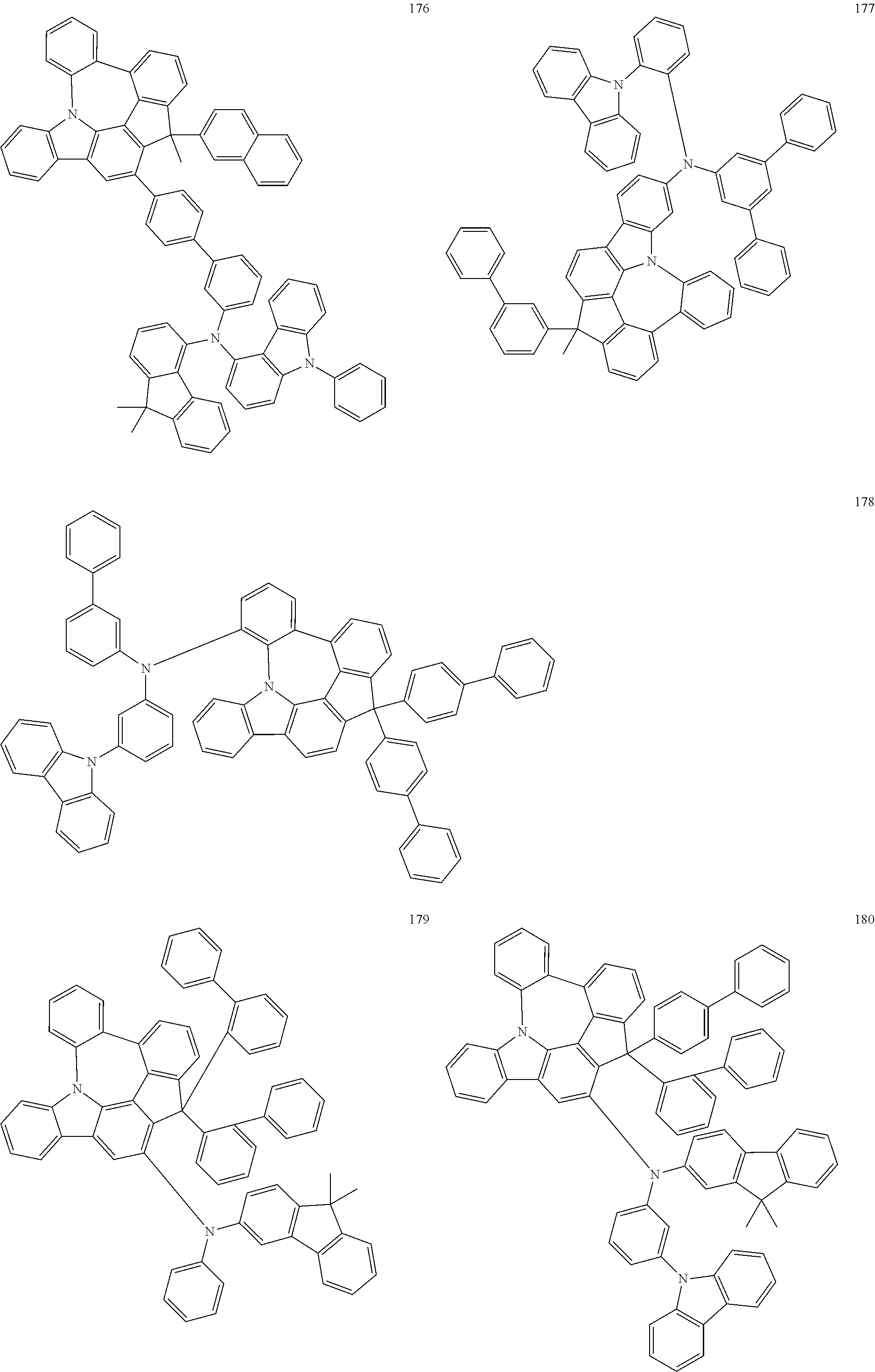

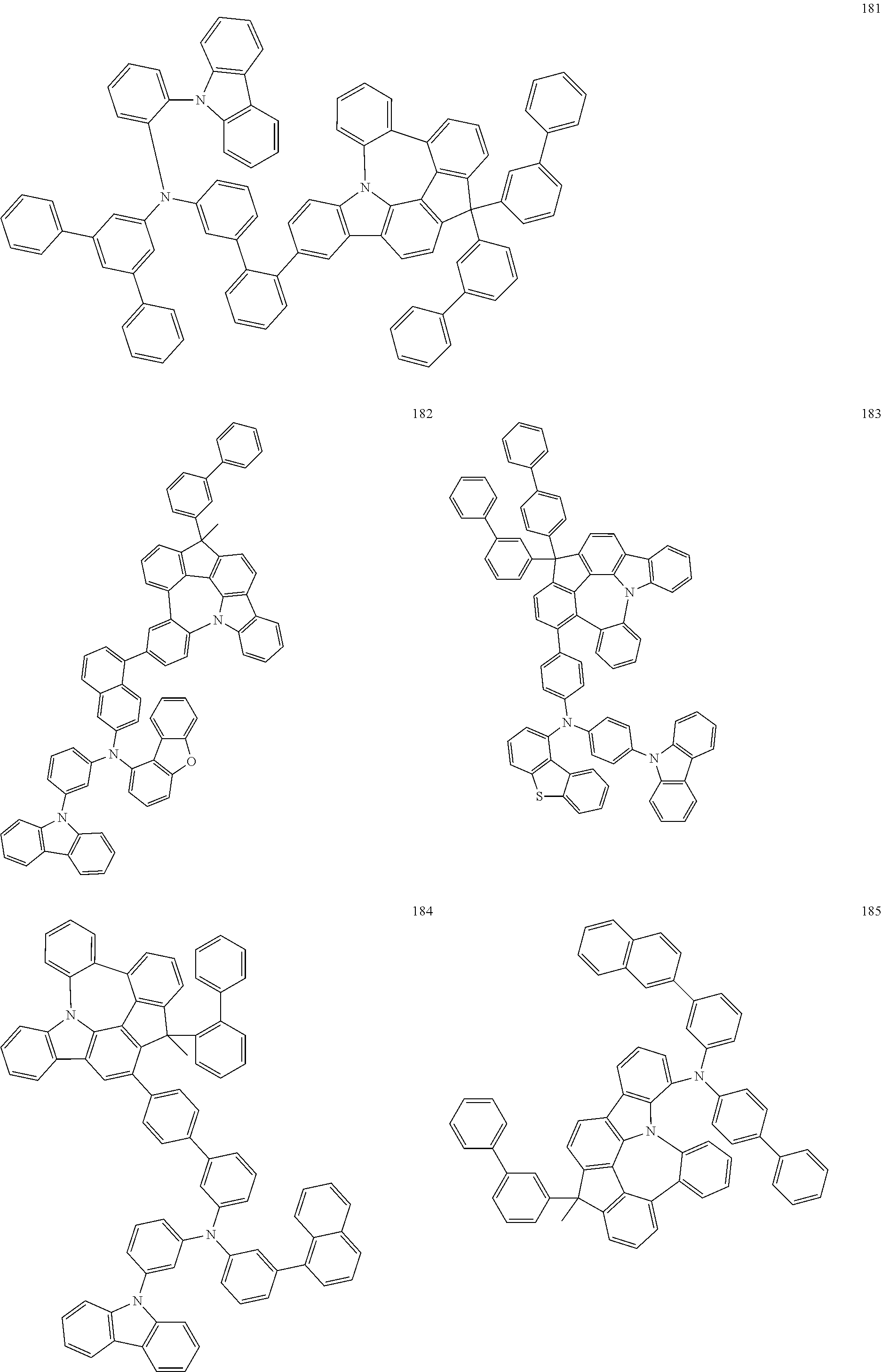

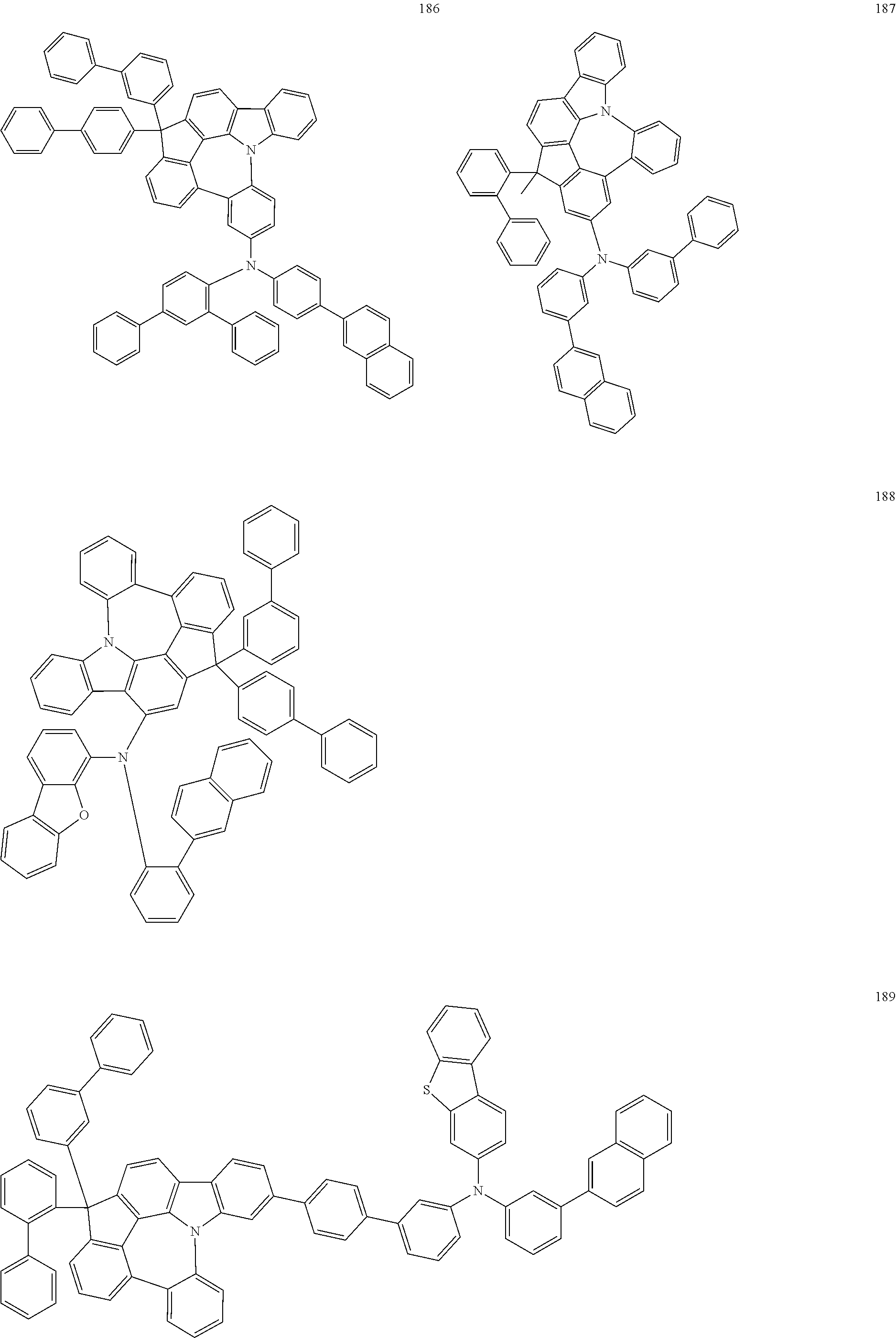

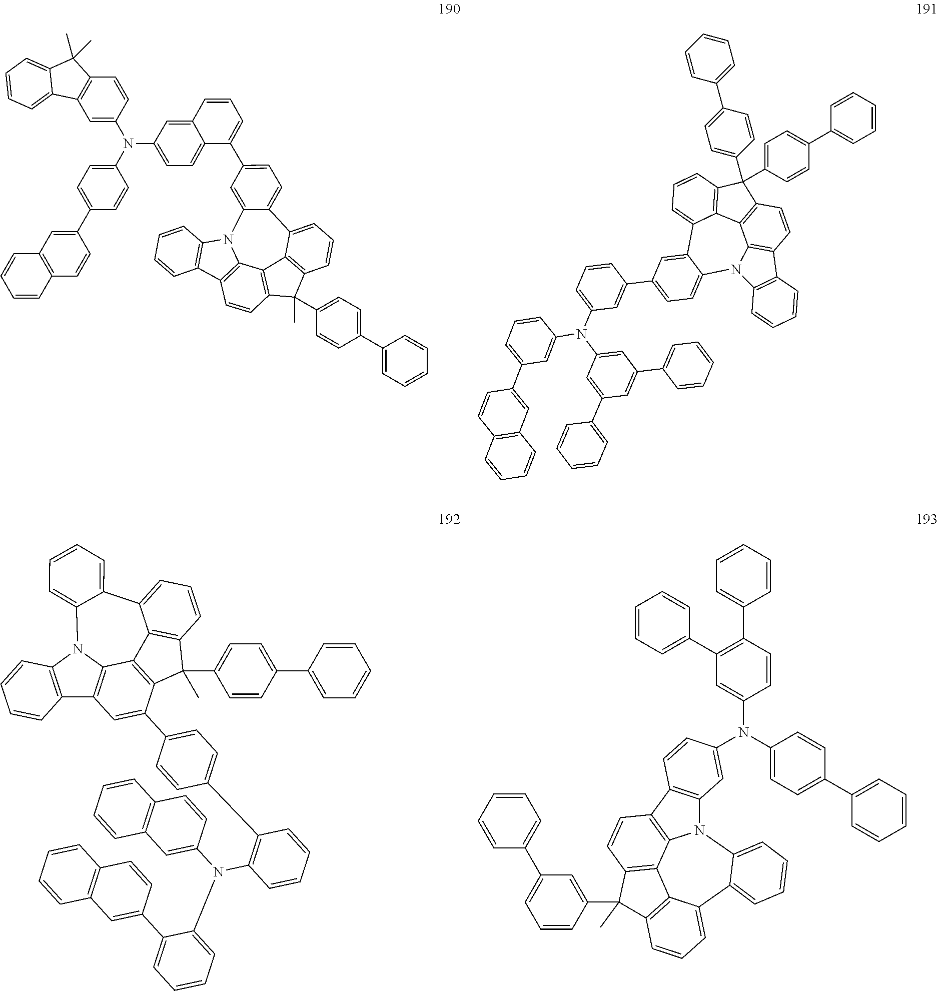

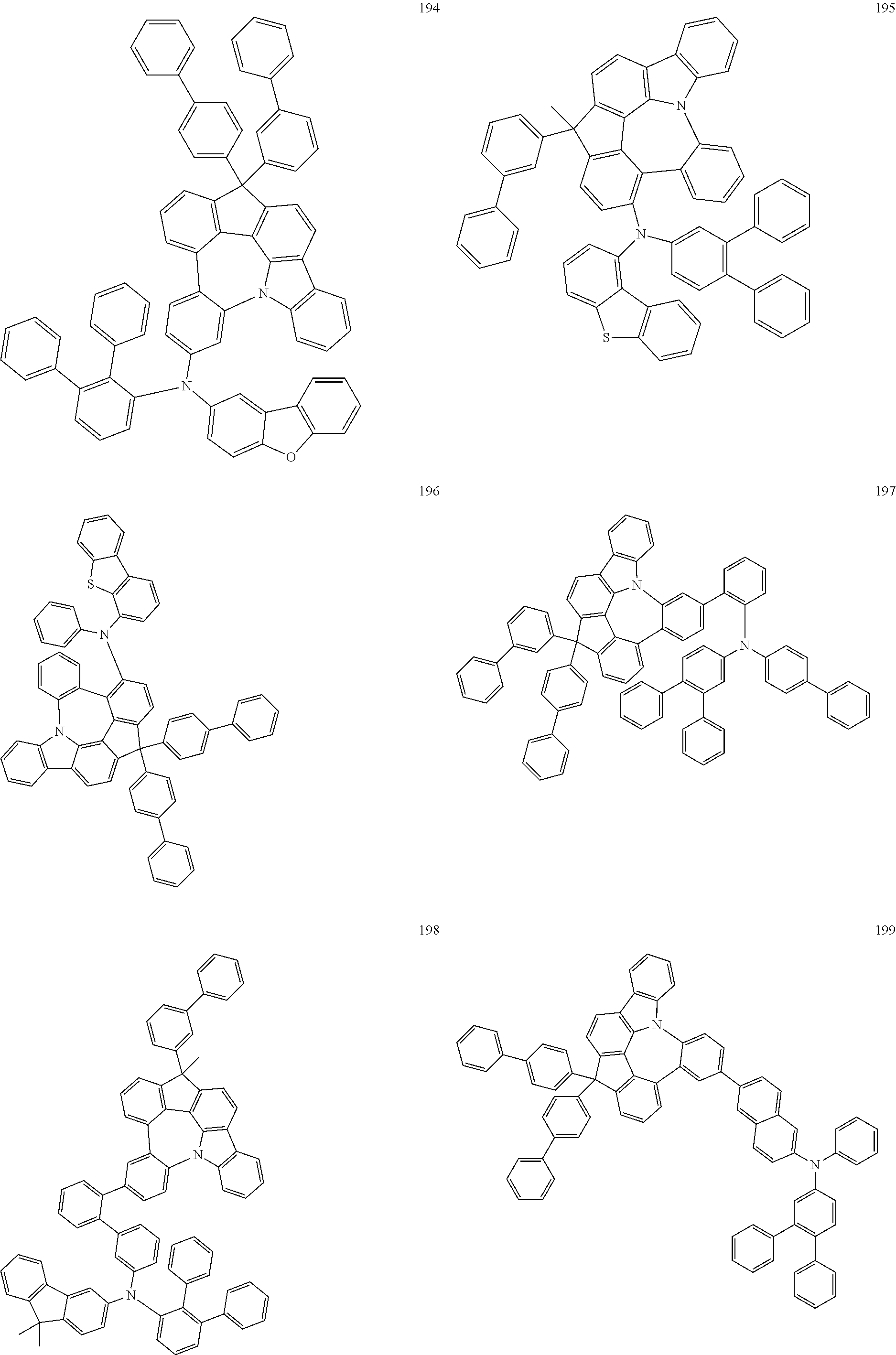

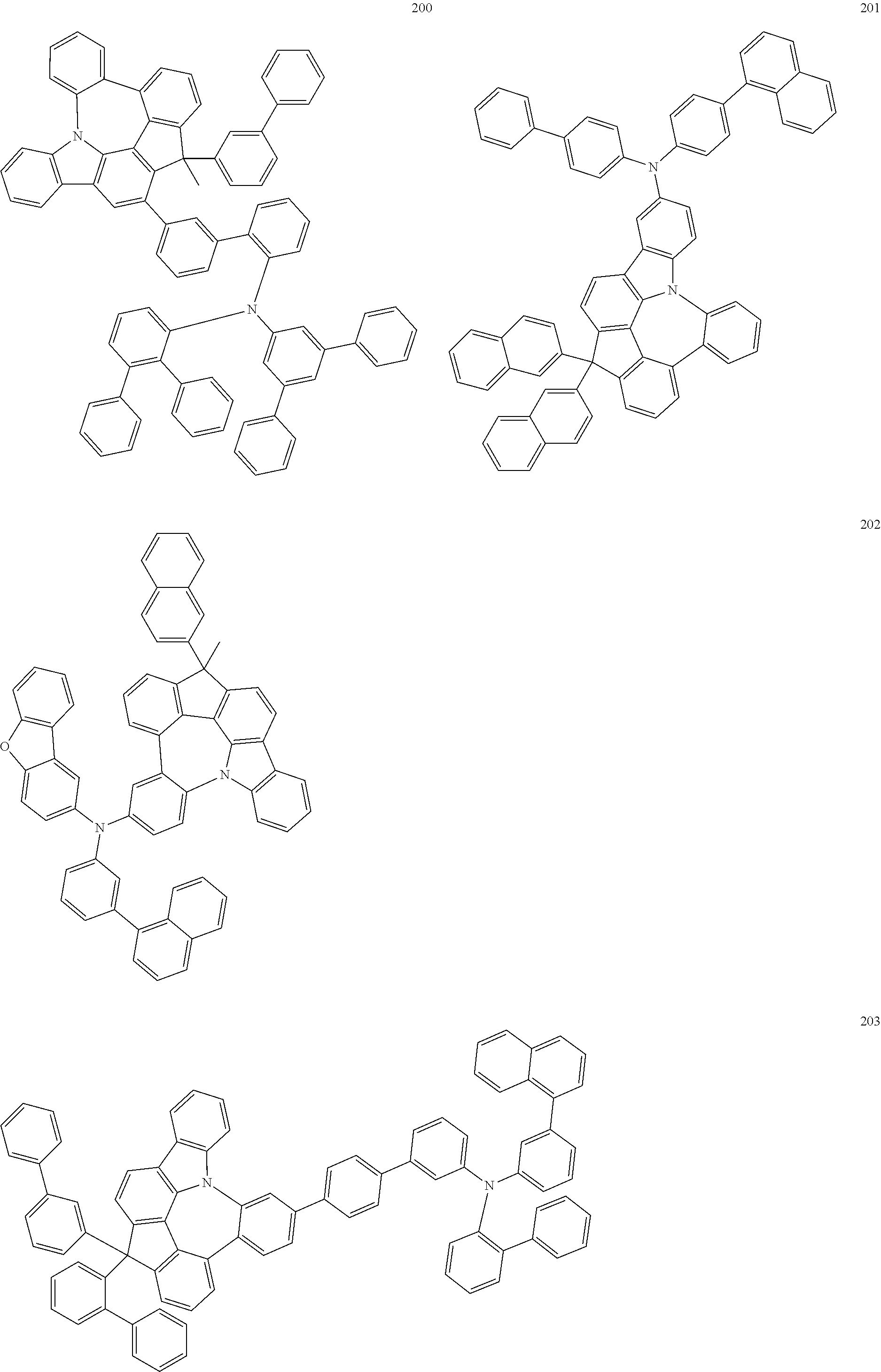

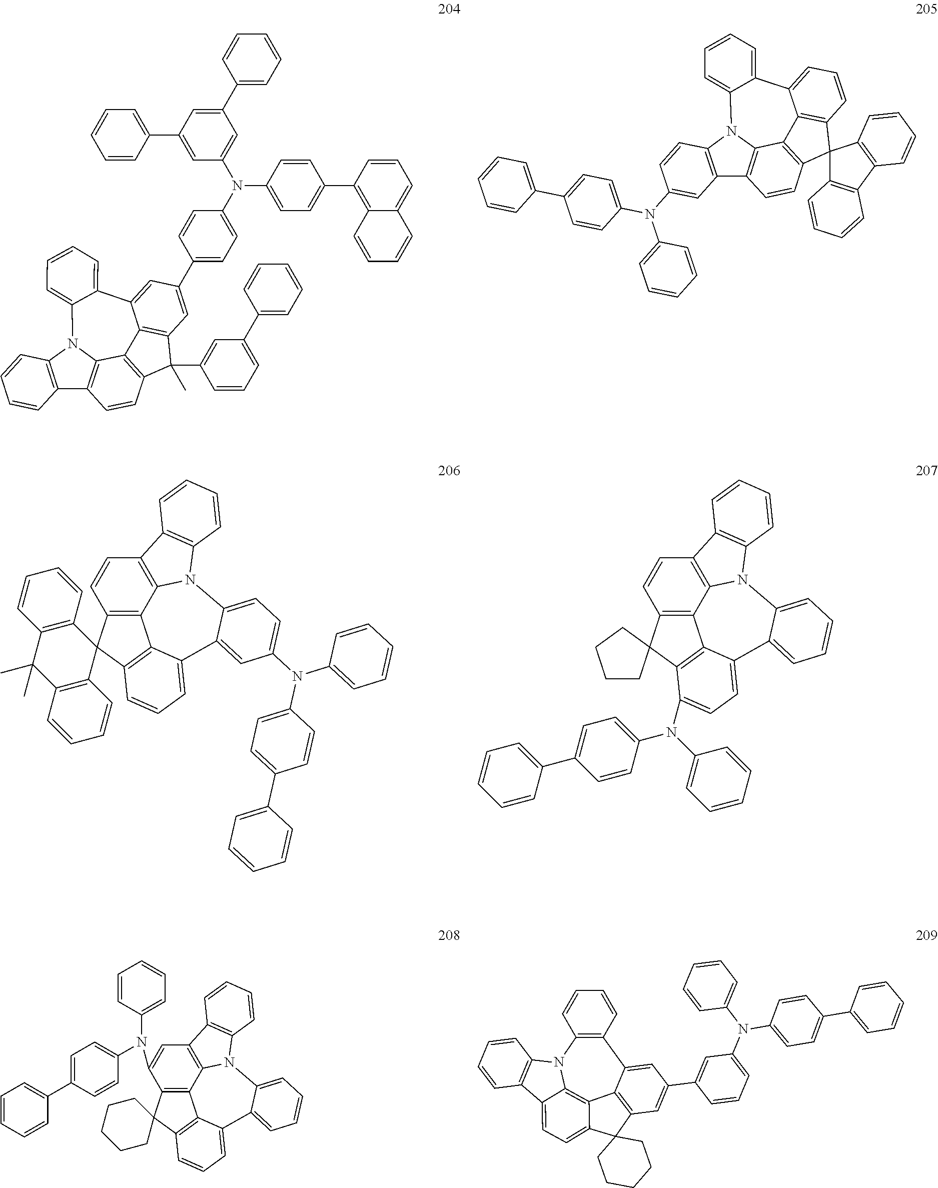

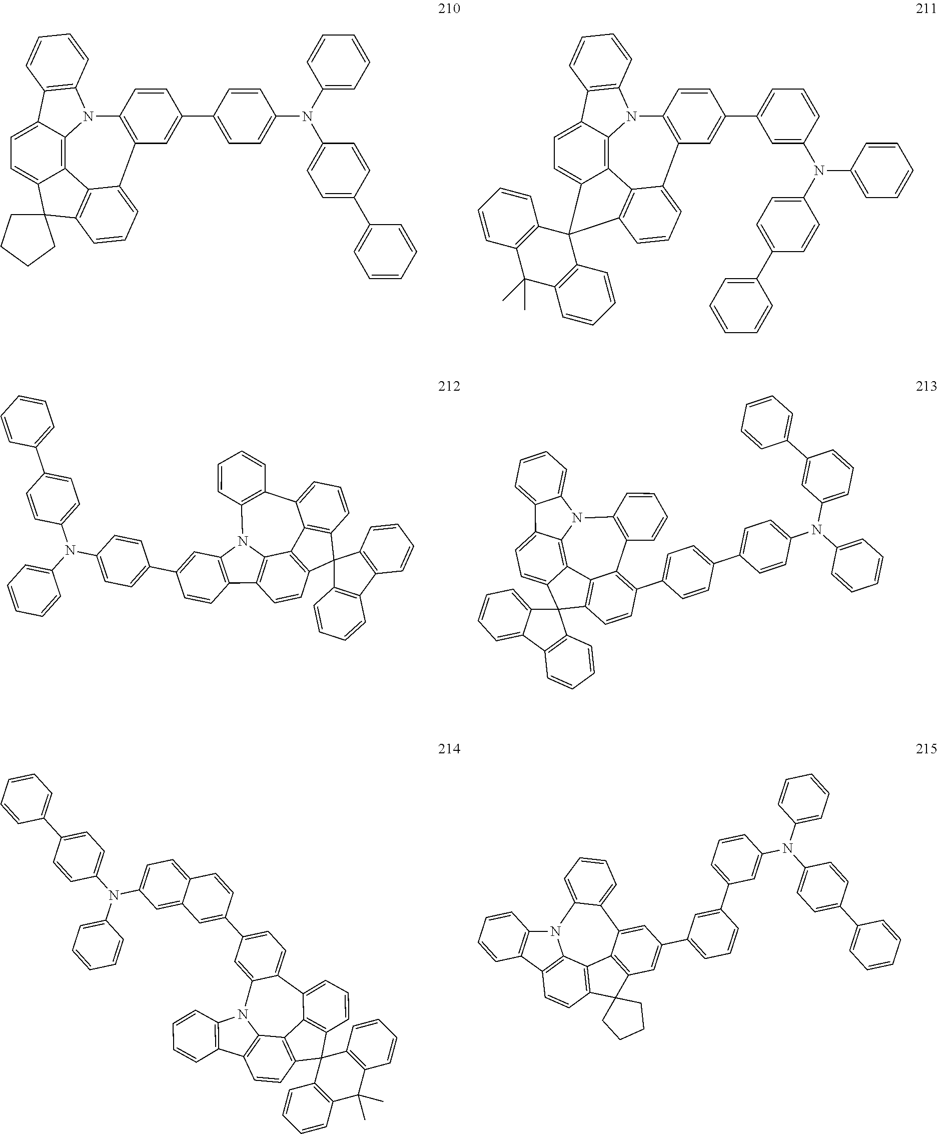

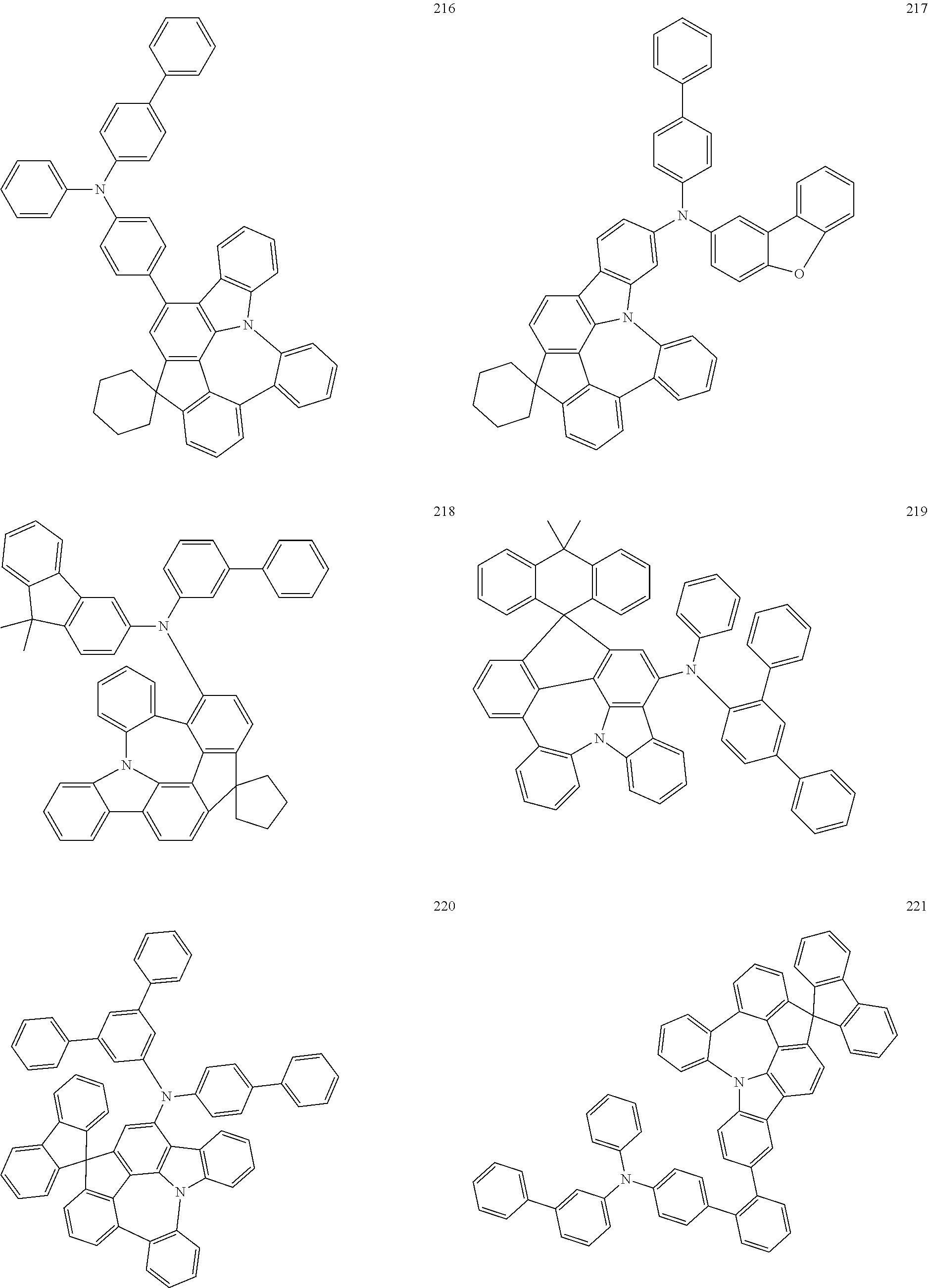

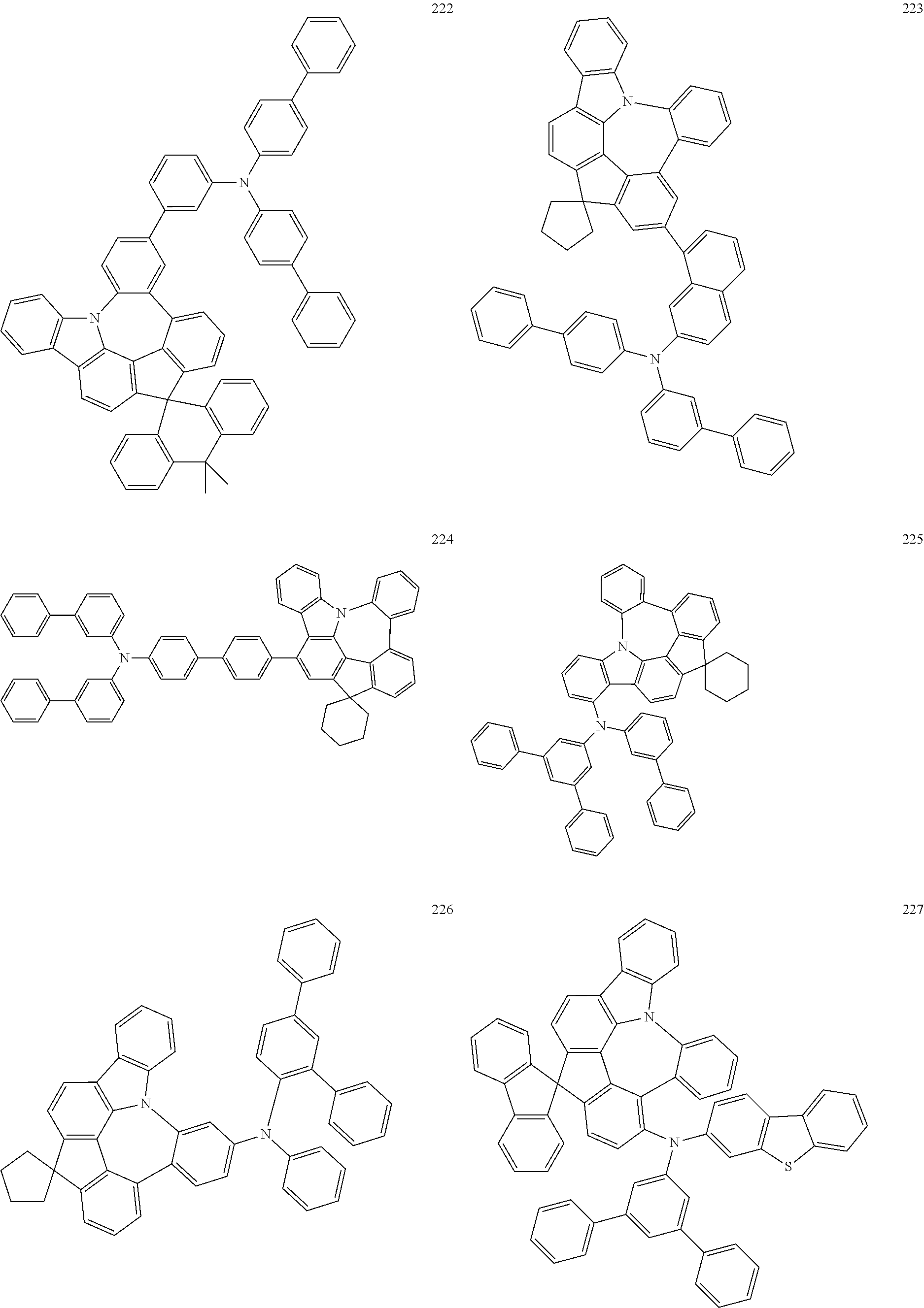

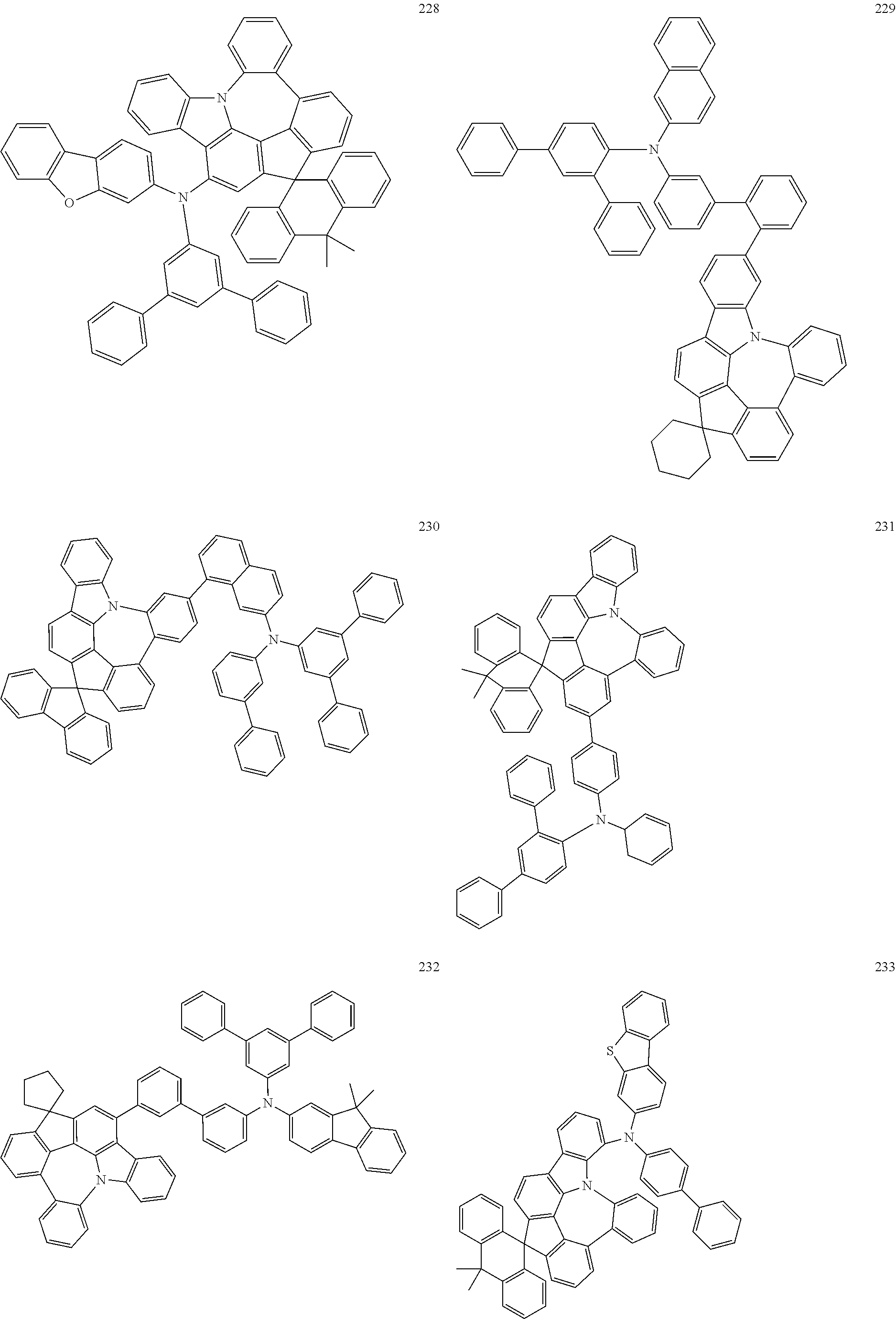

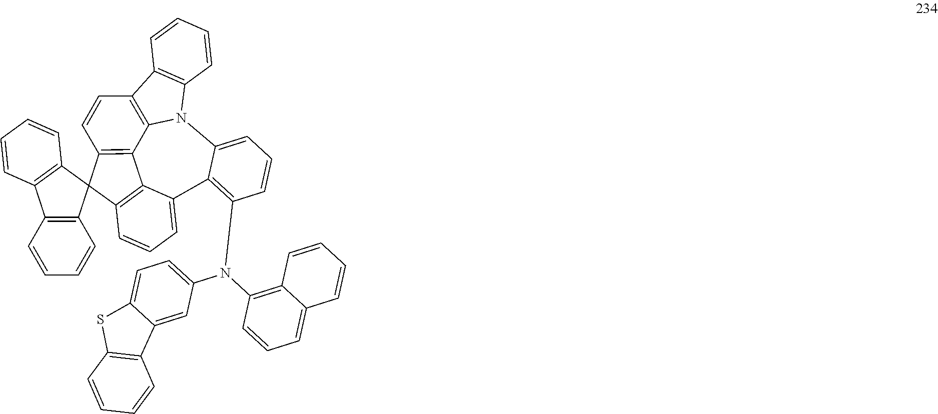 [SMILES: c1ccc2c(c1)-c1ccccc1C21c2cccc3c2-c2c1ccc1c4ccccc4n(c21)-c1cccc(N(c2ccc4sc5ccccc5c4c2)c2cccc4ccccc24)c1-3]

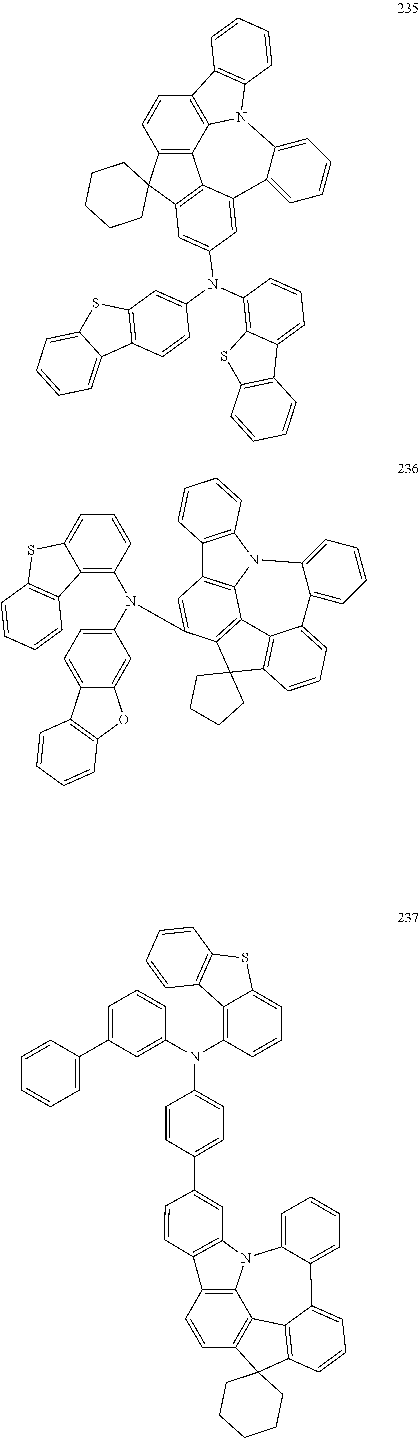 [SMILES: c1ccc2c(c1)-c1cc(N(c3ccc4c(c3)sc3ccccc34)c3cccc4c3sc3ccccc34)cc3c1-c1c(ccc4c5ccccc5n-2c14)C31CCCCC1 ; c1ccc2c(c1)-c1cccc3c1-c1c(c(N(c4ccc5c(c4)oc4ccccc45)c4cccc5sc6ccccc6c45)cc4c5ccccc5n-2c14)C31CCCC1 ; c1ccc(-c2cccc(N(c3ccc(-c4ccc5c6ccc7c8c6n(c5c4)-c4ccccc4-c4cccc(c4-8)C74CCCCC4)cc3)c3cccc4sc5ccccc5c34)c2)cc1]

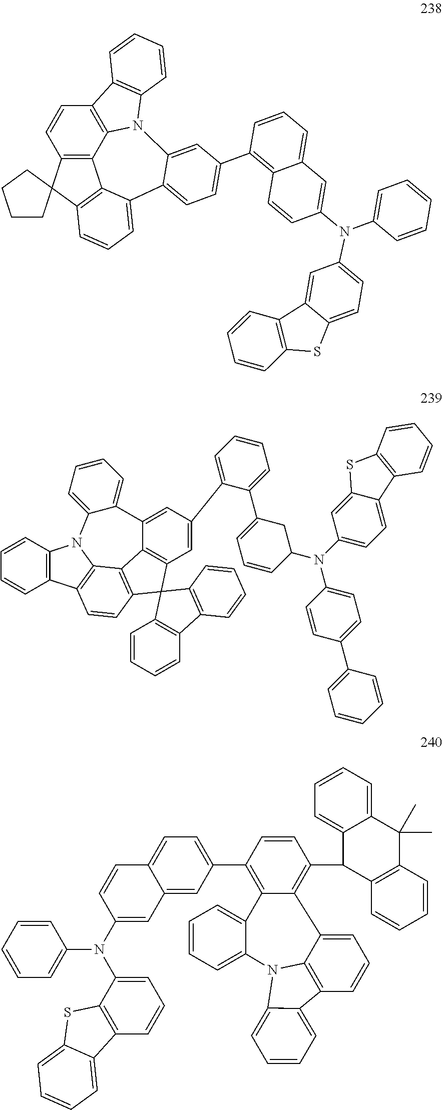 [SMILES: c1ccc(N(c2ccc3c(-c4ccc5c(c4)-n4c6ccccc6c6ccc7c(c64)-c4c-5cccc4C74CCCC4)cccc3c2)c2ccc3sc4ccccc4c3c2)cc1 ; C1=CC(N(c2ccc(-c3ccccc3)cc2)c2ccc3c(c2)sc2ccccc23)CC(c2ccccc2-c2cc3c4c(c2)C2(c5ccccc5-c5ccccc52)c2ccc5c6ccccc6n(c5c2-4)-c2ccccc2-3)=C1 ; CC1(C)c2ccccc2C(c2ccc(-c3ccc4ccc(N(c5ccccc5)c5cccc6c5sc5ccccc56)cc4c3)c3c2-c2cccc4c5ccccc5n(c24)-c2ccccc2-3)c2ccccc21]

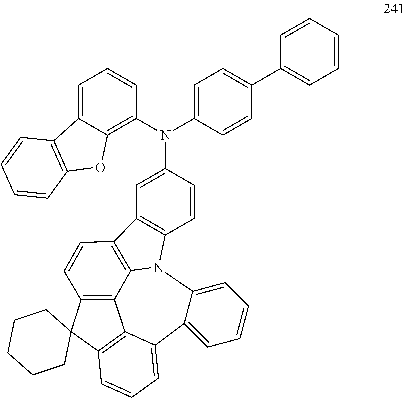 [SMILES: c1ccc(-c2ccc(N(c3ccc4c(c3)c3ccc5c6c3n4-c3ccccc3-c3cccc(c3-6)C53CCCCC3)c3cccc4c3oc3ccccc34)cc2)cc1]

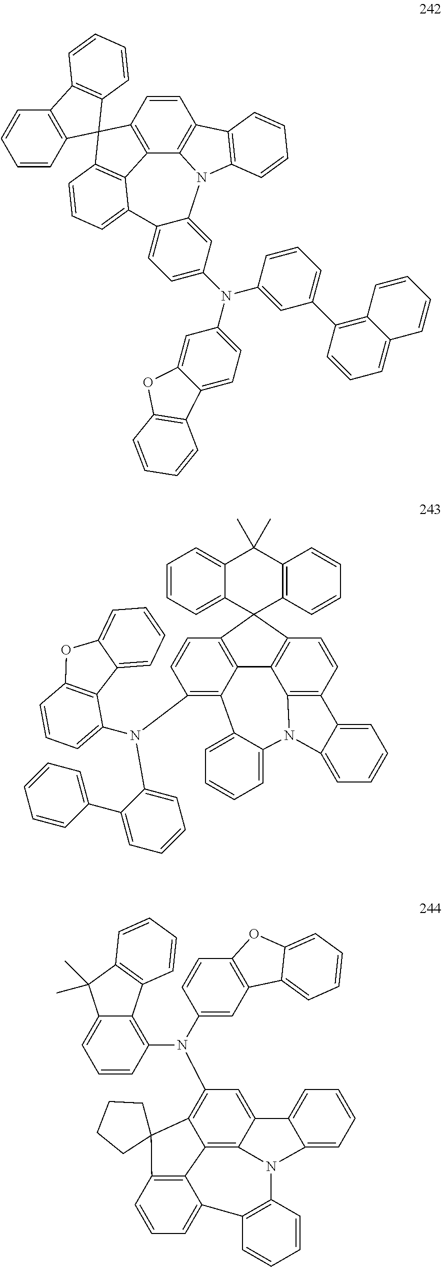 [SMILES: c1cc(-c2cccc3ccccc23)cc(N(c2ccc3c(c2)-n2c4ccccc4c4ccc5c(c42)-c2c-3cccc2C52c3ccccc3-c3ccccc32)c2ccc3c(c2)oc2ccccc23)c1 ; CC1(C)c2ccccc2C2(c3ccccc31)c1ccc(N(c3ccccc3-c3ccccc3)c3cccc4oc5ccccc5c34)c3c1-c1c2ccc2c4ccccc4n(c12)-c1ccccc1-3 ; CC1(C)c2ccccc2-c2c(N(c3ccc4oc5ccccc5c4c3)c3cc4c5ccccc5n5c4c4c3C3(CCCC3)c3cccc(c3-4)-c3ccccc3-5)cccc21]

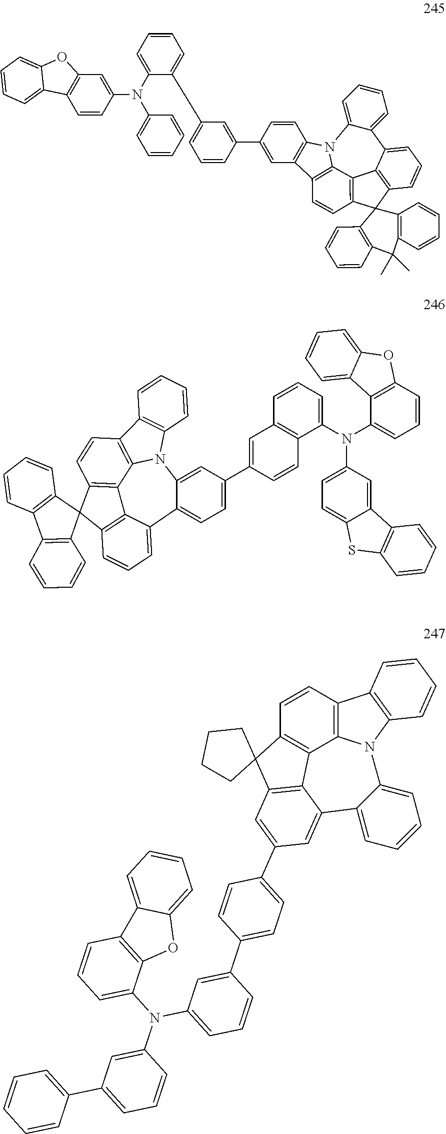 [SMILES: CC1(C)c2ccccc2C2(c3ccccc31)c1cccc3c1-c1c2ccc2c4cc(-c5cccc(-c6ccccc6N(c6ccccc6)c6ccc7c(c6)oc6ccccc67)c5)ccc4n(c12)-c1ccccc1-3 ; c1ccc2c(c1)-c1ccccc1C21c2cccc3c2-c2c1ccc1c4ccccc4n(c21)-c1cc(-c2ccc4c(N(c5ccc6sc7ccccc7c6c5)c5cccc6oc7ccccc7c56)cccc4c2)ccc1-3 ; c1ccc(-c2cccc(N(c3cccc(-c4ccc(-c5cc6c7c(c5)C5(CCCC5)c5ccc8c9ccccc9n(c8c5-7)-c5ccccc5-6)cc4)c3)c3cccc4c3oc3ccccc34)c2)cc1]

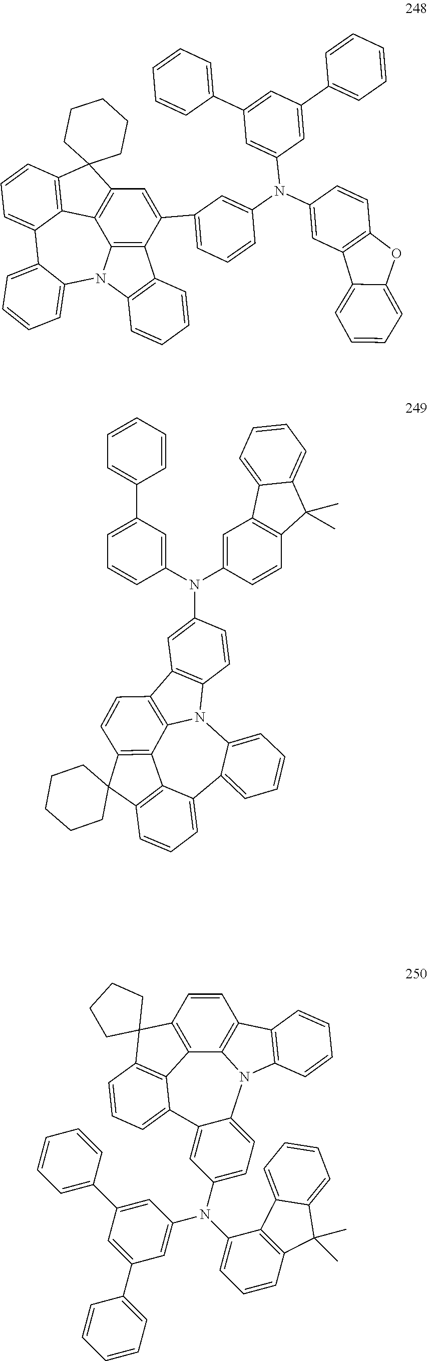 [SMILES: c1ccc(-c2cc(-c3ccccc3)cc(N(c3cccc(-c4cc5c6c7c4c4ccccc4n7-c4ccccc4-c4cccc(c4-6)C54CCCCC4)c3)c3ccc4oc5ccccc5c4c3)c2)cc1 ; CC1(C)c2ccccc2-c2cc(N(c3cccc(-c4ccccc4)c3)c3ccc4c(c3)c3ccc5c6c3n4-c3ccccc3-c3cccc(c3-6)C53CCCCC3)ccc21 ; CC1(C)c2ccccc2-c2c(N(c3cc(-c4ccccc4)cc(-c4ccccc4)c3)c3ccc4c(c3)-c3cccc5c3-c3c(ccc6c7ccccc7n-4c36)C53CCCC3)cccc21]

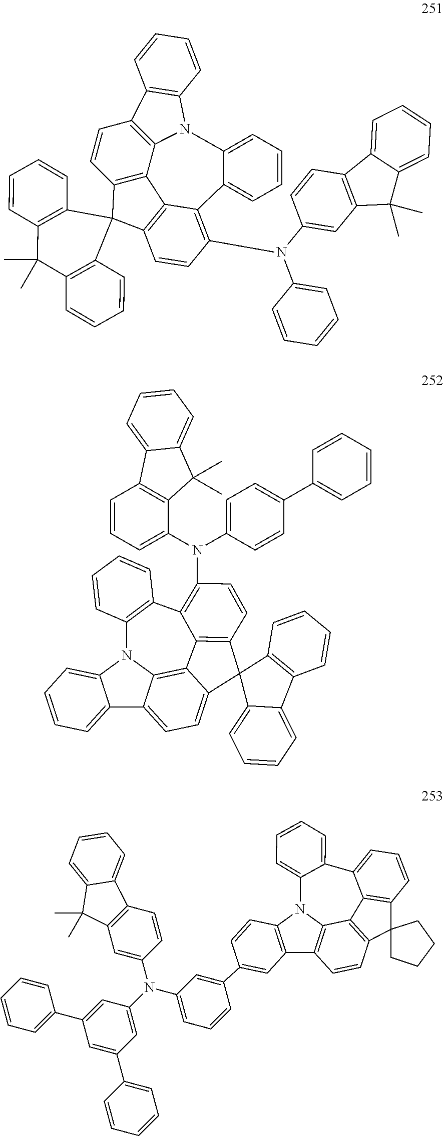 [SMILES: CC1(C)c2ccccc2-c2ccc(N(c3ccccc3)c3ccc4c5c3-c3ccccc3-n3c6ccccc6c6ccc(c-5c63)C43c4ccccc4C(C)(C)c4ccccc43)cc21 ; CC1(C)c2ccccc2-c2cccc(N(c3ccc(-c4ccccc4)cc3)c3ccc4c5c3-c3ccccc3-n3c6ccccc6c6ccc(c-5c63)C43c4ccccc4-c4ccccc43)c21 ; CC1(C)c2ccccc2-c2ccc(N(c3cccc(-c4ccc5c(c4)c4ccc6c7c4n5-c4ccccc4-c4cccc(c4-7)C64CCCC4)c3)c3cc(-c4ccccc4)cc(-c4ccccc4)c3)cc21]

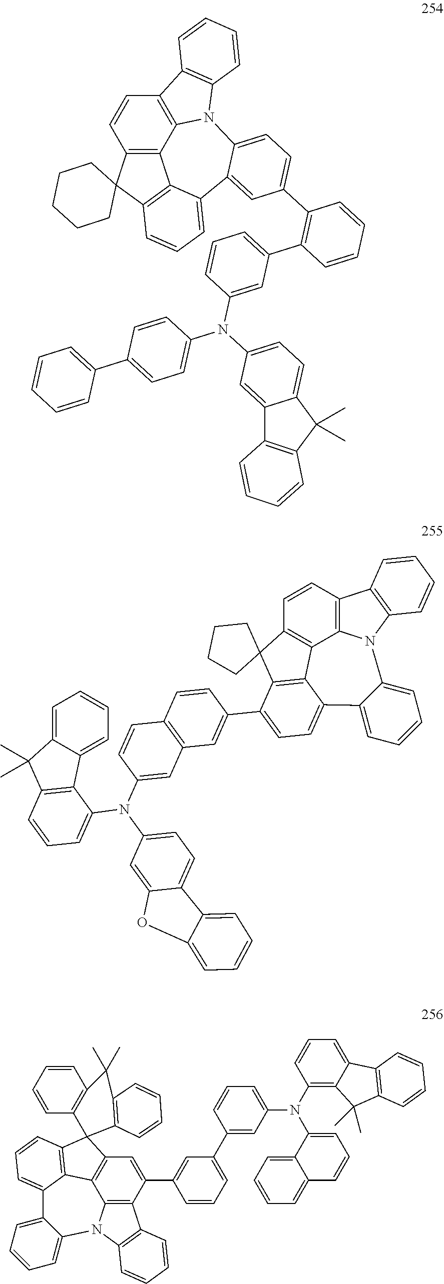 [SMILES: CC1(C)c2ccccc2-c2cc(N(c3ccc(-c4ccccc4)cc3)c3cccc(-c4ccccc4-c4ccc5c(c4)-c4cccc6c4-c4c(ccc7c8ccccc8n-5c47)C64CCCCC4)c3)ccc21 ; CC1(C)c2ccccc2-c2c(N(c3ccc4ccc(-c5ccc6c7c5C5(CCCC5)c5ccc8c9ccccc9n(c8c5-7)-c5ccccc5-6)cc4c3)c3ccc4c(c3)oc3ccccc34)cccc21 ; CC1(C)c2ccccc2C2(c3ccccc31)c1cccc3c1-c1c2cc(-c2cccc(-c4cccc(N(c5cccc6c5C(C)(C)c5ccccc5-6)c5cccc6ccccc56)c4)c2)c2c4ccccc4n(c12)-c1ccccc1-3]

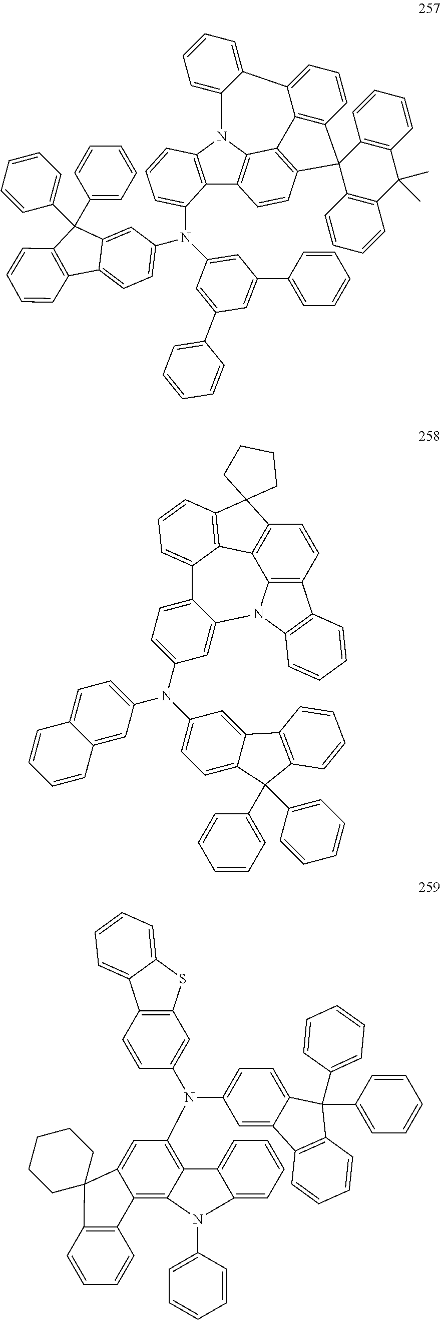 [SMILES: CC1(C)c2ccccc2C2(c3ccccc31)c1cccc3c1-c1c2ccc2c4c(N(c5cc(-c6ccccc6)cc(-c6ccccc6)c5)c5ccc6c(c5)C(c5ccccc5)(c5ccccc5)c5ccccc5-6)cccc4n(c12)-c1ccccc1-3 ; c1ccc(C2(c3ccccc3)c3ccccc3-c3cc(N(c4ccc5c(c4)-n4c6ccccc6c6ccc7c(c64)-c4c-5cccc4C74CCCC4)c4ccc5ccccc5c4)ccc32)cc1 ; c1ccc(-n2c3ccccc3c3c(N(c4ccc5c(c4)-c4ccccc4C5(c4ccccc4)c4ccccc4)c4ccc5c(c4)sc4ccccc45)cc4c(c32)-c2ccccc2C42CCCCC2)cc1]

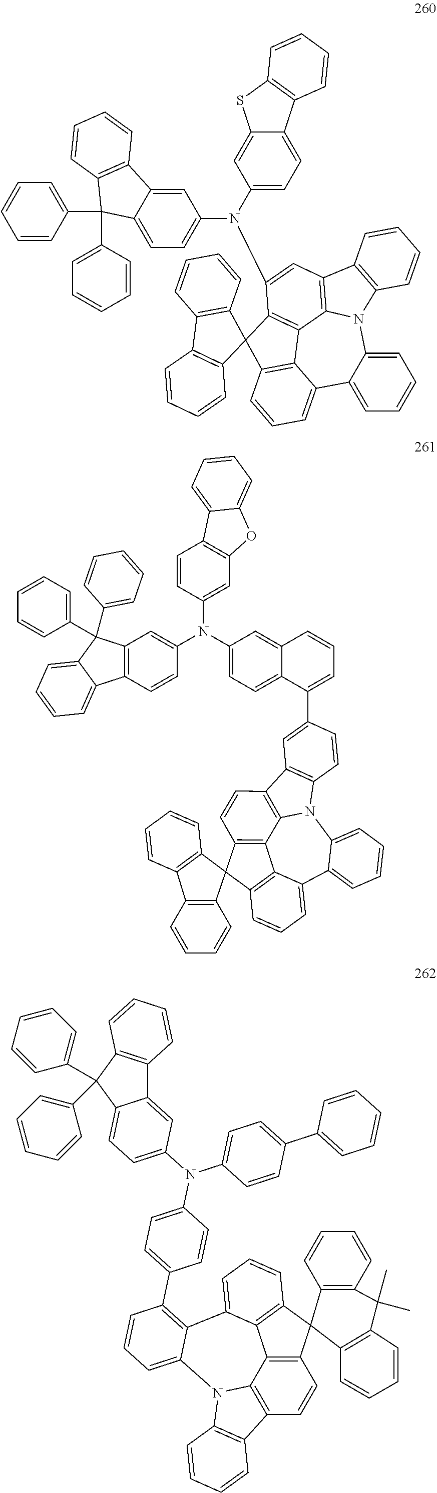

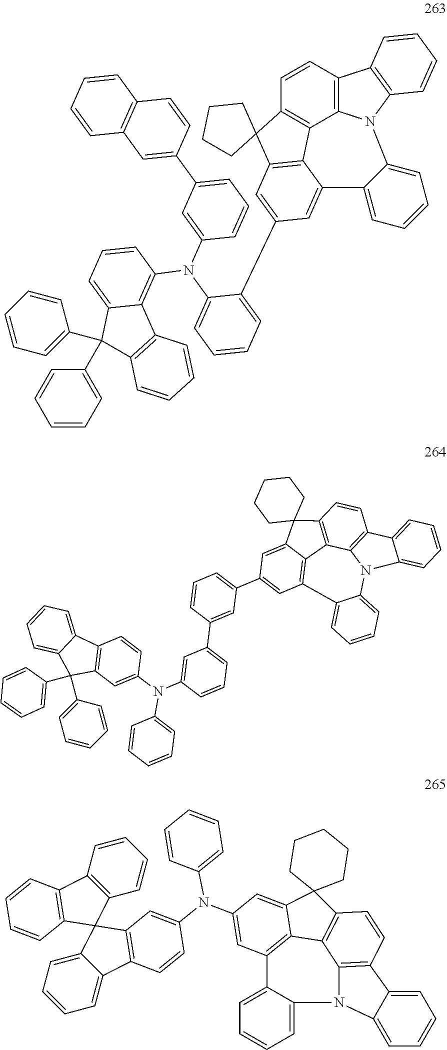 [SMILES: c1ccc(C2(c3ccccc3)c3ccccc3-c3c(N(c4cccc(-c5ccc6ccccc6c5)c4)c4ccccc4-c4cc5c6c(c4)C4(CCCC4)c4ccc7c8ccccc8n(c7c4-6)-c4ccccc4-5)cccc32)cc1 ; c1ccc(N(c2cccc(-c3cccc(-c4cc5c6c(c4)C4(CCCCC4)c4ccc7c8ccccc8n(c7c4-6)-c4ccccc4-5)c3)c2)c2ccc3c(c2)C(c2ccccc2)(c2ccccc2)c2ccccc2-3)cc1 ; c1ccc(N(c2cc3c4c(c2)C2(CCCCC2)c2ccc5c6ccccc6n(c5c2-4)-c2ccccc2-3)c2ccc3c(c2)C2(c4ccccc4-c4ccccc42)c2ccccc2-3)cc1]

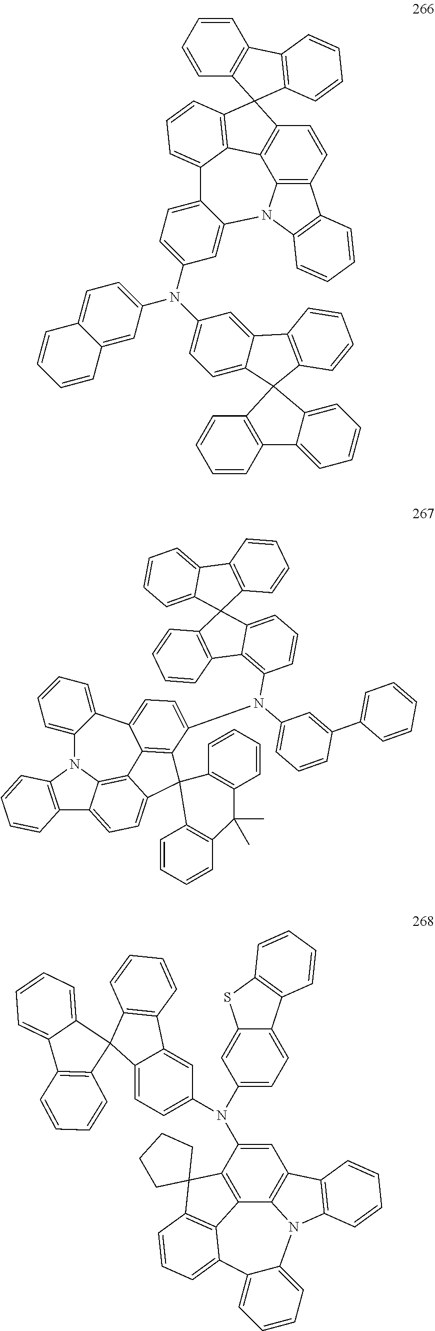 [SMILES: c1ccc2c(c1)-c1ccccc1C21c2ccccc2-c2cc(N(c3ccc4c(c3)-n3c5ccccc5c5ccc6c(c53)-c3c-4cccc3C63c4ccccc4-c4ccccc43)c3ccc4ccccc4c3)ccc21 ; CC1(C)c2ccccc2C2(c3ccccc31)c1ccc3c4ccccc4n4c3c1-c1c(ccc(N(c3cccc(-c5ccccc5)c3)c3cccc5c3-c3ccccc3C53c5ccccc5-c5ccccc53)c12)-c1ccccc1-4 ; c1ccc2c(c1)-c1cccc3c1-c1c(c(N(c4ccc5c(c4)-c4ccccc4C54c5ccccc5-c5ccccc54)c4ccc5c(c4)sc4ccccc45)cc4c5ccccc5n-2c14)C31CCCC1]

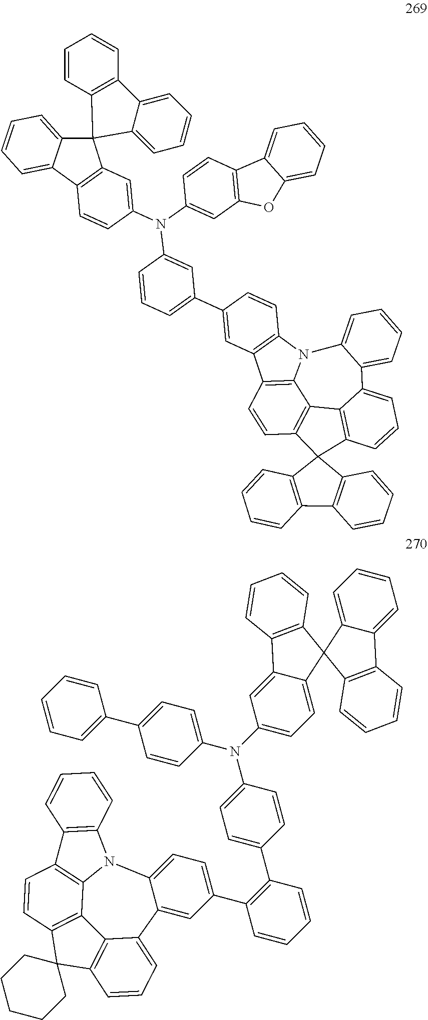

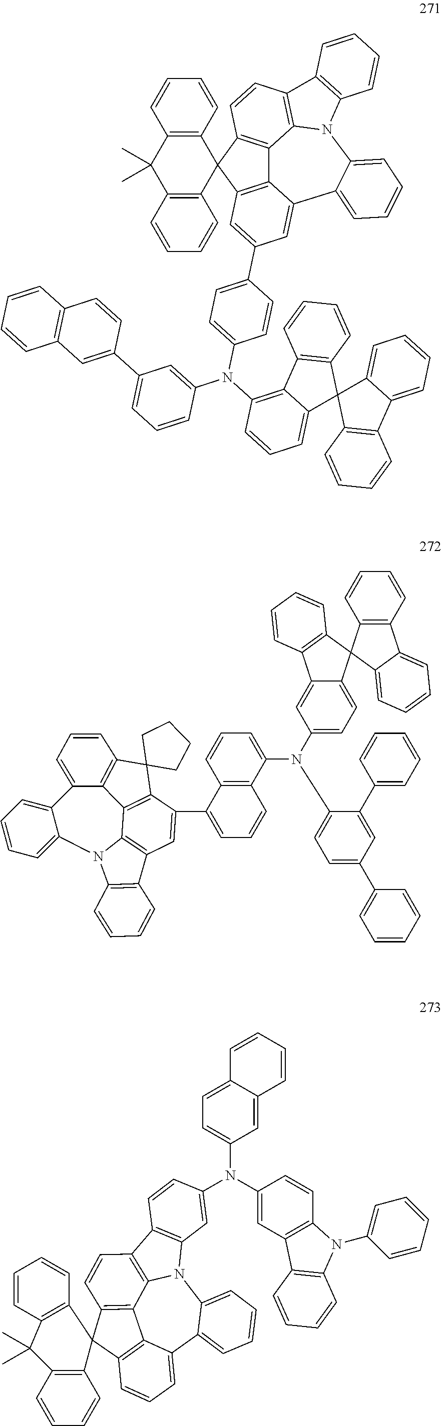 [SMILES: CC1(C)c2ccccc2C2(c3ccccc31)c1cc(-c3ccc(N(c4cccc(-c5ccc6ccccc6c5)c4)c4cccc5c4-c4ccccc4C54c5ccccc5-c5ccccc54)cc3)cc3c1-c1c2ccc2c4ccccc4n(c12)-c1ccccc1-3 ; c1ccc(-c2ccc(N(c3ccc4c(c3)-c3ccccc3C43c4ccccc4-c4ccccc43)c3cccc4c(-c5cc6c7ccccc7n7c6c6c5C5(CCCC5)c5cccc(c5-6)-c5ccccc5-7)cccc34)c(-c3ccccc3)c2)cc1 ; CC1(C)c2ccccc2C2(c3ccccc31)c1cccc3c1-c1c2ccc2c4ccc(N(c5ccc6ccccc6c5)c5ccc6c(c5)c5ccccc5n6-c5ccccc5)cc4n(c12)-c1ccccc1-3]

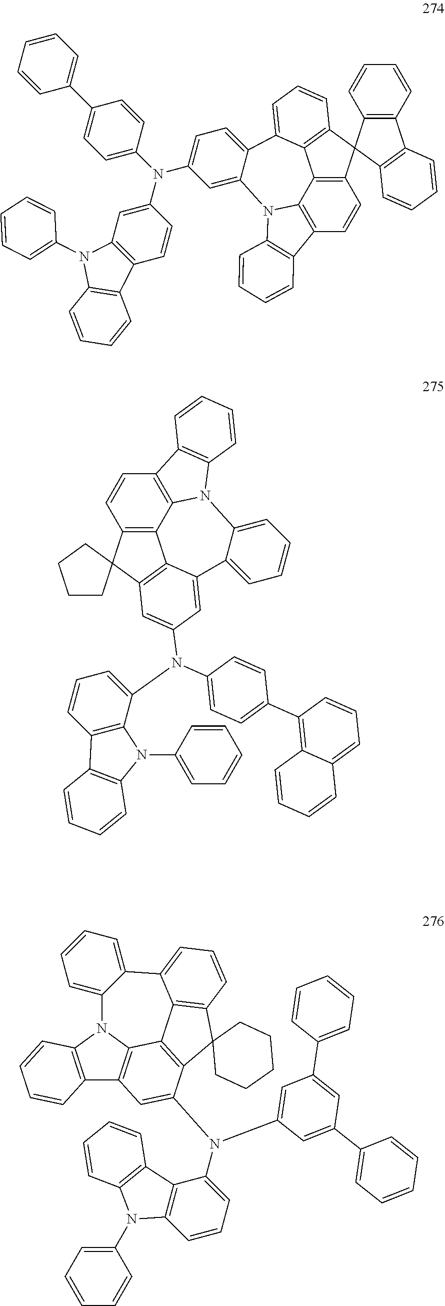 [SMILES: c1ccc(-c2ccc(N(c3ccc4c(c3)-n3c5ccccc5c5ccc6c(c53)-c3c-4cccc3C63c4ccccc4-c4ccccc43)c3ccc4c5ccccc5n(-c5ccccc5)c4c3)cc2)cc1 ; c1ccc(-n2c3ccccc3c3cccc(N(c4ccc(-c5cccc6ccccc56)cc4)c4cc5c6c(c4)C4(CCCC4)c4ccc7c8ccccc8n(c7c4-6)-c4ccccc4-5)c32)cc1 ; c1ccc(-c2cc(-c3ccccc3)cc(N(c3cc4c5ccccc5n5c4c4c3C3(CCCCC3)c3cccc(c3-4)-c3ccccc3-5)c3cccc4c3c3ccccc3n4-c3ccccc3)c2)cc1]

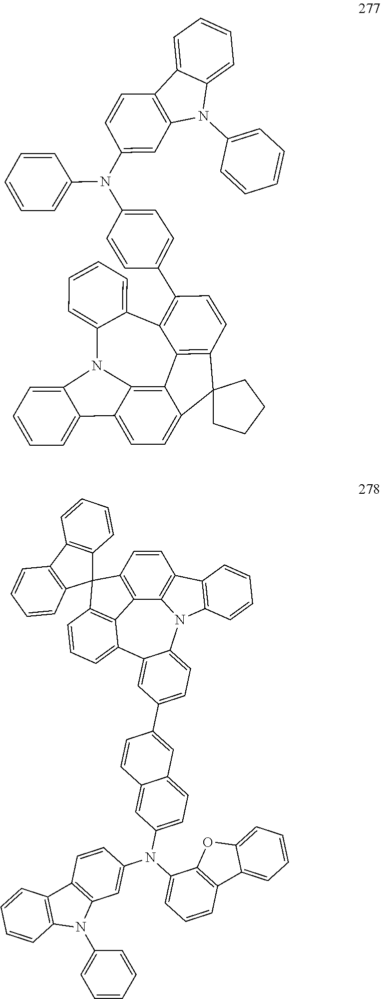 [SMILES: c1ccc(N(c2ccc(-c3ccc4c5c3-c3ccccc3-n3c6ccccc6c6ccc(c-5c63)C43CCCC3)cc2)c2ccc3c4ccccc4n(-c4ccccc4)c3c2)cc1 ; c1ccc(-n2c3ccccc3c3ccc(N(c4ccc5cc(-c6ccc7c(c6)-c6cccc8c6-c6c(ccc9c%10ccccc%10n-7c69)C86c7ccccc7-c7ccccc76)ccc5c4)c4cccc5c4oc4ccccc45)cc32)cc1]

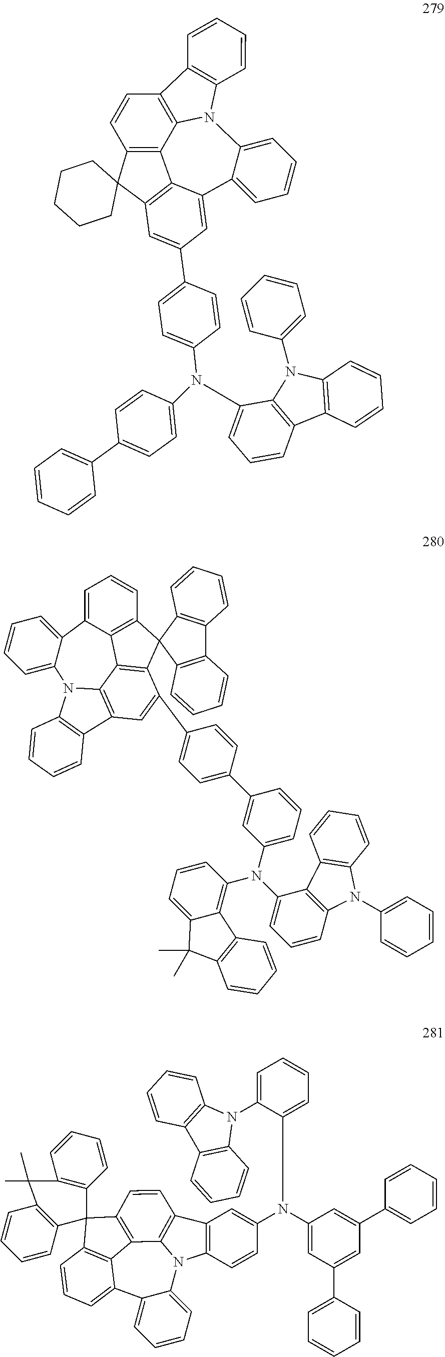 [SMILES: c1ccc(-c2ccc(N(c3ccc(-c4cc5c6c(c4)C4(CCCCC4)c4ccc7c8ccccc8n(c7c4-6)-c4ccccc4-5)cc3)c3cccc4c5ccccc5n(-c5ccccc5)c34)cc2)cc1 ; CC1(C)c2ccccc2-c2c(N(c3cccc(-c4ccc(-c5cc6c7ccccc7n7c6c6c5C5(c8ccccc8-c8ccccc85)c5cccc(c5-6)-c5ccccc5-7)cc4)c3)c3cccc4c3c3ccccc3n4-c3ccccc3)cccc21 ; CC1(C)c2ccccc2C2(c3ccccc31)c1cccc3c1-c1c2ccc2c4cc(N(c5cc(-c6ccccc6)cc(-c6ccccc6)c5)c5ccccc5-n5c6ccccc6c6ccccc65)ccc4n(c12)-c1ccccc1-3]

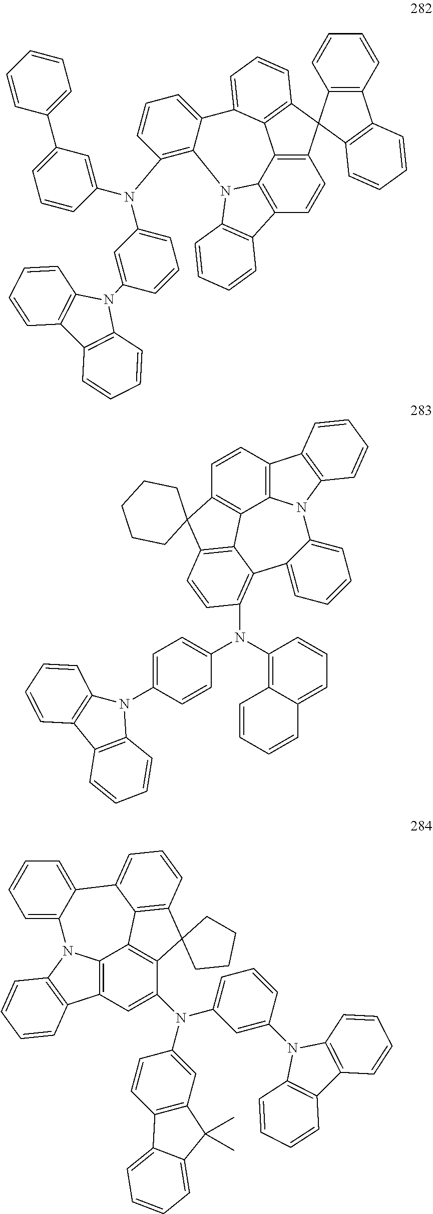 [SMILES: c1ccc(-c2cccc(N(c3cccc(-n4c5ccccc5c5ccccc54)c3)c3cccc4c3-n3c5ccccc5c5ccc6c(c53)-c3c-4cccc3C63c4ccccc4-c4ccccc43)c2)cc1 ; c1ccc2c(c1)-c1c(N(c3ccc(-n4c5ccccc5c5ccccc54)cc3)c3cccc4ccccc34)ccc3c1-c1c(ccc4c5ccccc5n-2c14)C31CCCCC1 ; CC1(C)c2ccccc2-c2ccc(N(c3cccc(-n4c5ccccc5c5ccccc54)c3)c3cc4c5ccccc5n5c4c4c3C3(CCCC3)c3cccc(c3-4)-c3ccccc3-5)cc21]

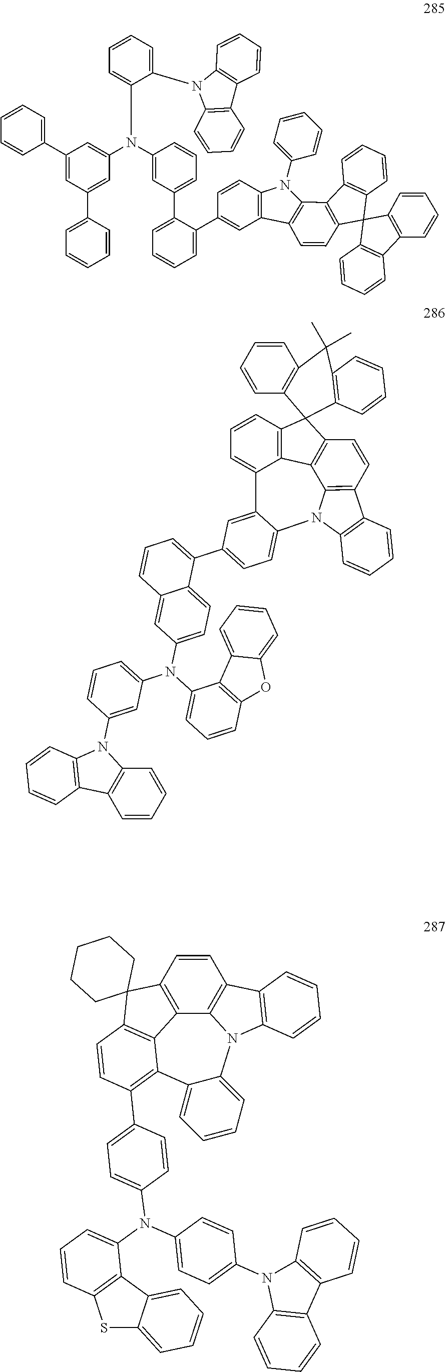

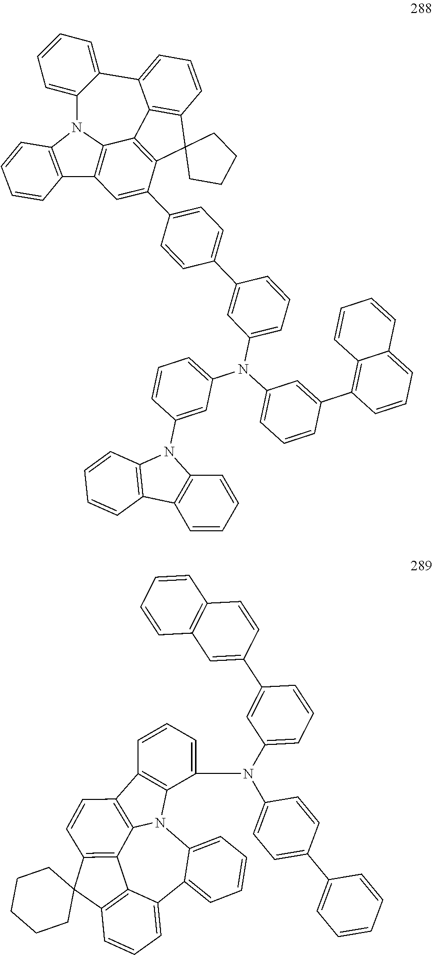

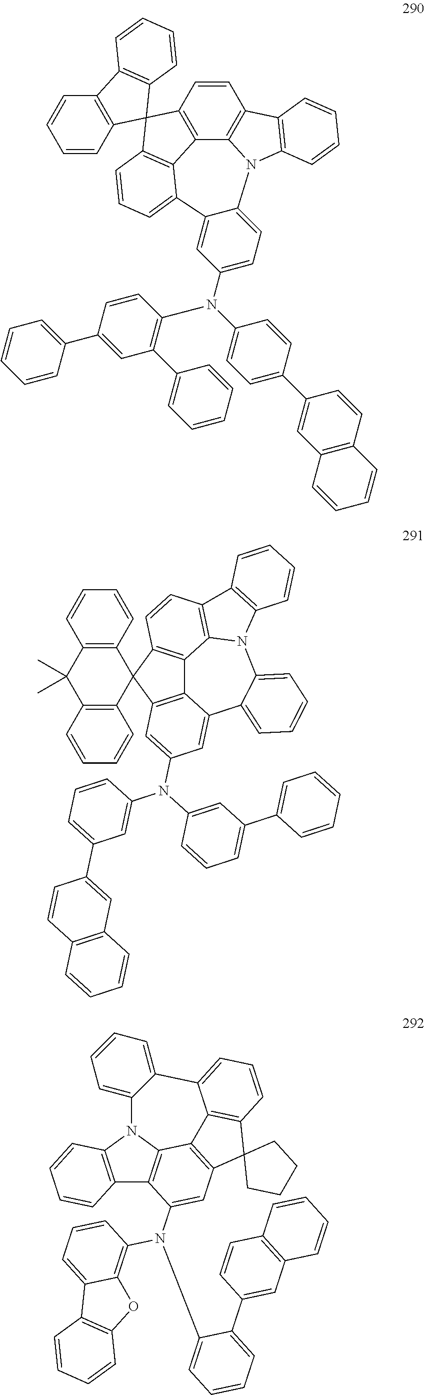

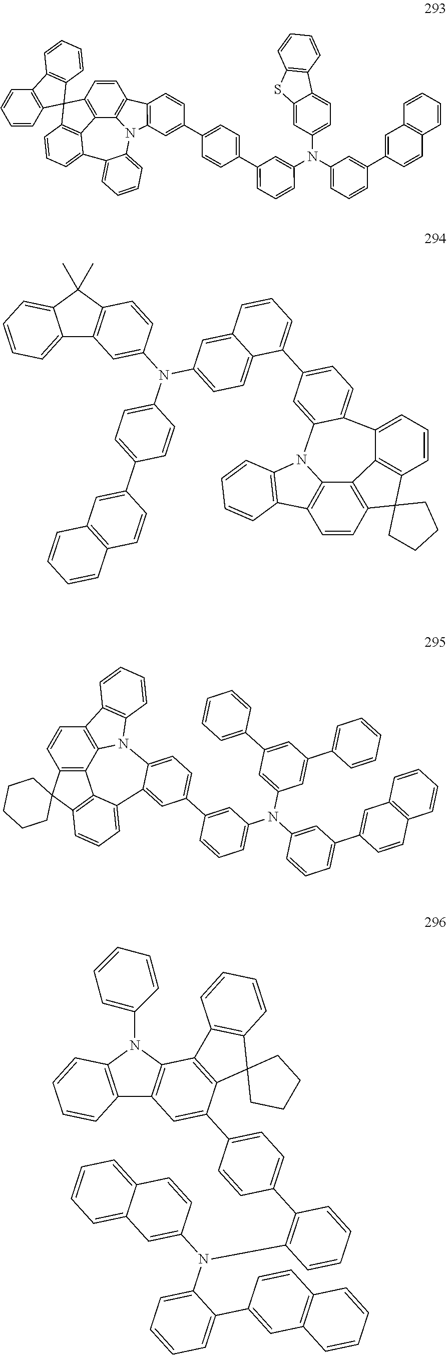

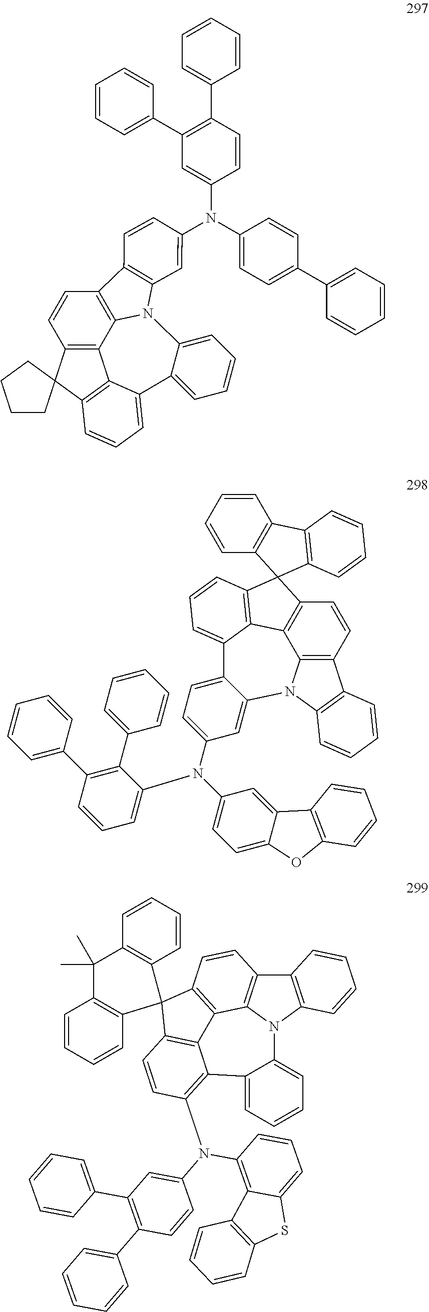 [SMILES: c1ccc(-c2ccc(N(c3ccc(-c4ccccc4)c(-c4ccccc4)c3)c3ccc4c5ccc6c7c5n(c4c3)-c3ccccc3-c3cccc(c3-7)C63CCCC3)cc2)cc1 ; c1ccc(-c2cccc(N(c3ccc4c(c3)-n3c5ccccc5c5ccc6c(c53)-c3c-4cccc3C63c4ccccc4-c4ccccc43)c3ccc4oc5ccccc5c4c3)c2-c2ccccc2)cc1 ; CC1(C)c2ccccc2C2(c3ccccc31)c1ccc(N(c3ccc(-c4ccccc4)c(-c4ccccc4)c3)c3cccc4sc5ccccc5c34)c3c1-c1c2ccc2c4ccccc4n(c12)-c1ccccc1-3]

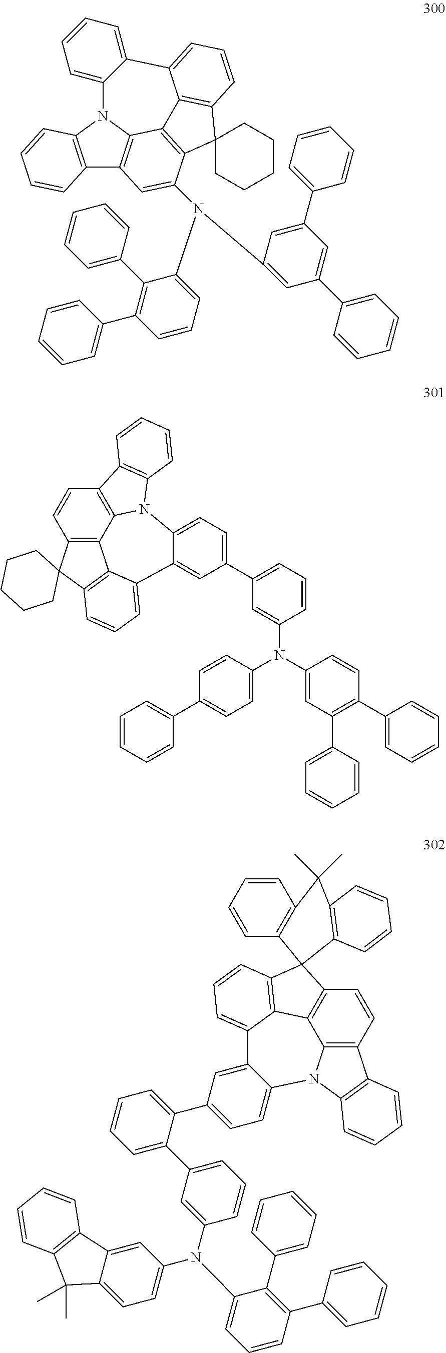 [SMILES: c1ccc(-c2cc(-c3ccccc3)cc(N(c3cccc(-c4ccccc4)c3-c3ccccc3)c3cc4c5ccccc5n5c4c4c3C3(CCCCC3)c3cccc(c3-4)-c3ccccc3-5)c2)cc1 ; c1ccc(-c2ccc(N(c3cccc(-c4ccc5c(c4)-c4cccc6c4-c4c(ccc7c8ccccc8n-5c47)C64CCCCC4)c3)c3ccc(-c4ccccc4)c(-c4ccccc4)c3)cc2)cc1 ; CC1(C)c2ccccc2-c2cc(N(c3cccc(-c4ccccc4-c4ccc5c(c4)-c4cccc6c4-c4c(ccc7c8ccccc8n-5c47)C64c5ccccc5C(C)(C)c5ccccc54)c3)c3cccc(-c4ccccc4)c3-c3ccccc3)ccc21]

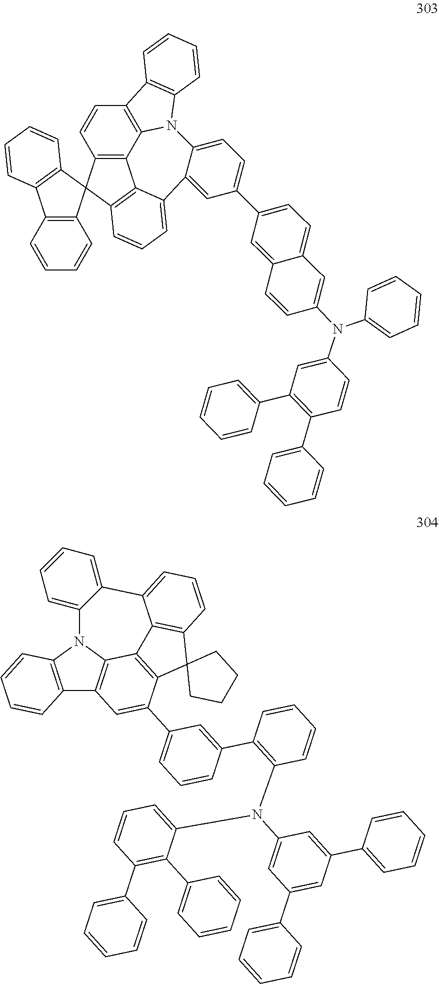 [SMILES: c1ccc(-c2ccc(N(c3ccccc3)c3ccc4cc(-c5ccc6c(c5)-c5cccc7c5-c5c(ccc8c9ccccc9n-6c58)C75c6ccccc6-c6ccccc65)ccc4c3)cc2-c2ccccc2)cc1 ; c1ccc(-c2cc(-c3ccccc3)cc(N(c3ccccc3-c3cccc(-c4cc5c6ccccc6n6c5c5c4C4(CCCC4)c4cccc(c4-5)-c4ccccc4-6)c3)c3cccc(-c4ccccc4)c3-c3ccccc3)c2)cc1]

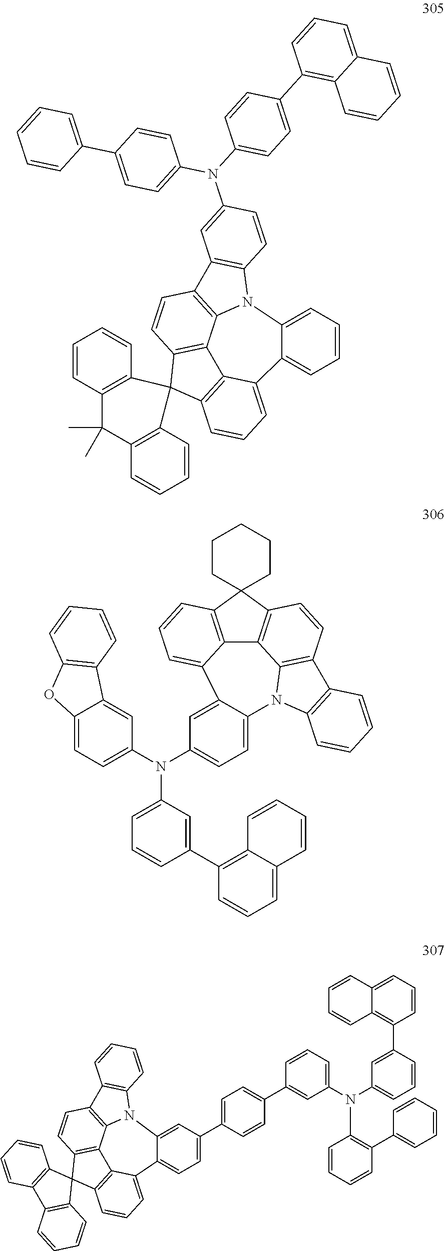 [SMILES: CC1(C)c2ccccc2C2(c3ccccc31)c1cccc3c1-c1c2ccc2c4cc(N(c5ccc(-c6ccccc6)cc5)c5ccc(-c6cccc7ccccc67)cc5)ccc4n(c12)-c1ccccc1-3 ; c1cc(-c2cccc3ccccc23)cc(N(c2ccc3c(c2)-c2cccc4c2-c2c(ccc5c6ccccc6n-3c25)C42CCCCC2)c2ccc3oc4ccccc4c3c2)c1 ; c1ccc(-c2ccccc2N(c2cccc(-c3ccc(-c4ccc5c(c4)-n4c6ccccc6c6ccc7c(c64)-c4c-5cccc4C74c5ccccc5-c5ccccc54)cc3)c2)c2cccc(-c3cccc4ccccc34)c2)cc1]

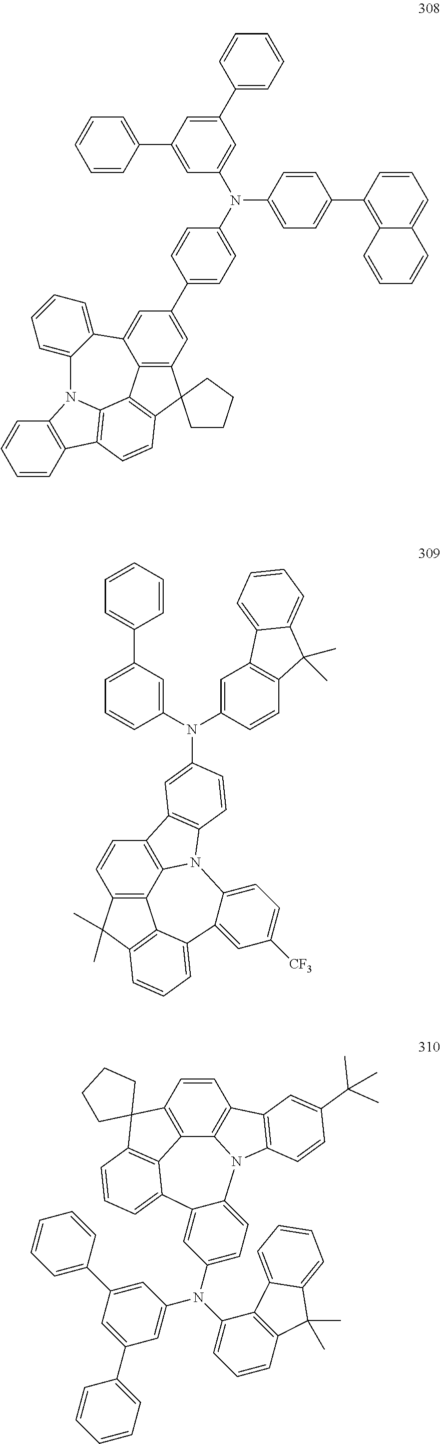

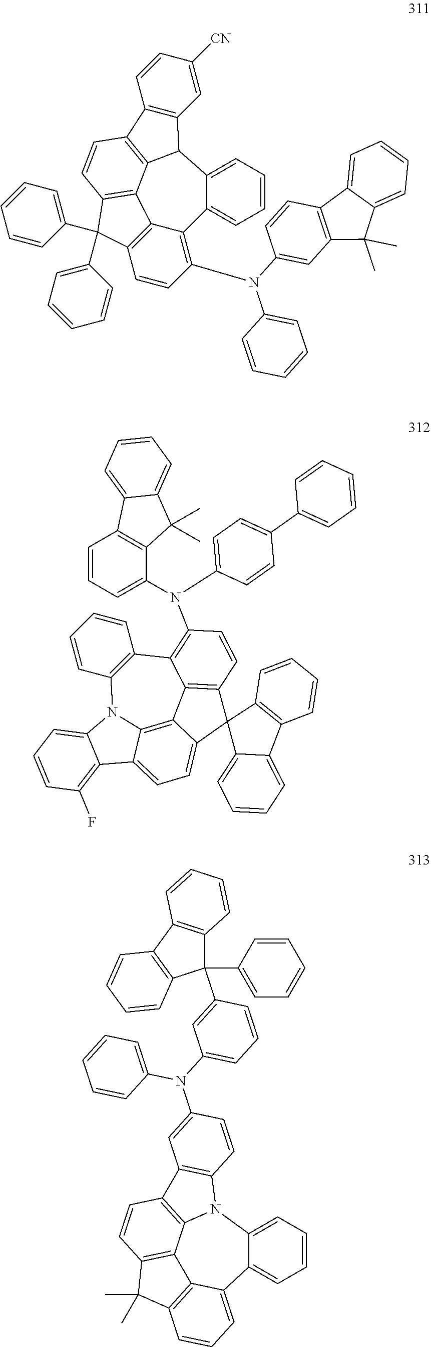 [SMILES: CC1(C)c2ccccc2-c2ccc(N(c3ccccc3)c3ccc4c5c3-c3ccccc3C3c6cc(C#N)ccc6-c6ccc(c-5c63)C4(c3ccccc3)c3ccccc3)cc21 ; CC1(C)c2ccccc2-c2cccc(N(c3ccc(-c4ccccc4)cc3)c3ccc4c5c3-c3ccccc3-n3c6cccc(F)c6c6ccc(c-5c63)C43c4ccccc4-c4ccccc43)c21 ; CC1(C)c2cccc3c2-c2c1ccc1c4cc(N(c5ccccc5)c5cccc(C6(c7ccccc7)c7ccccc7-c7ccccc76)c5)ccc4n(c21)-c1ccccc1-3]

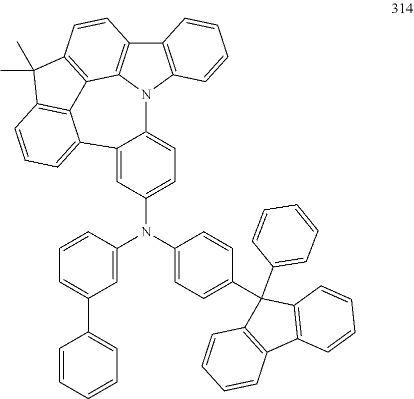 [SMILES: CC1(C)c2cccc3c2-c2c1ccc1c4ccccc4n(c21)-c1ccc(N(c2ccc(C4(c5ccccc5)c5ccccc5-c5ccccc54)cc2)c2cccc(-c4ccccc4)c2)cc1-3]

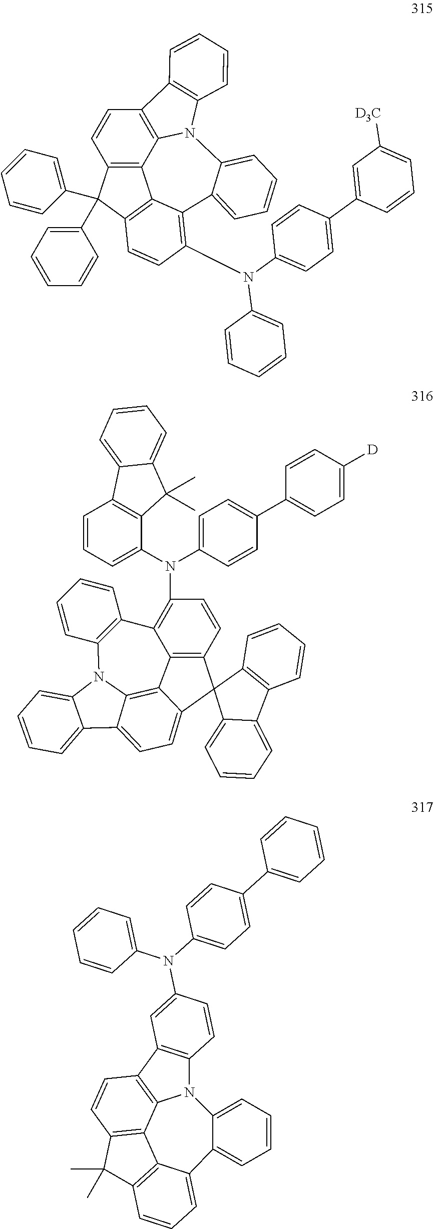 [SMILES: [2H]C([2H])([2H])c1cccc(-c2ccc(N(c3ccccc3)c3ccc4c5c3-c3ccccc3-n3c6ccccc6c6ccc(c-5c63)C4(c3ccccc3)c3ccccc3)cc2)c1 ; [2H]c1ccc(-c2ccc(N(c3ccc4c5c3-c3ccccc3-n3c6ccccc6c6ccc(c-5c63)C43c4ccccc4-c4ccccc43)c3cccc4c3C(C)(C)c3ccccc3-4)cc2)cc1 ; CC1(C)c2cccc3c2-c2c1ccc1c4cc(N(c5ccccc5)c5ccc(-c6ccccc6)cc5)ccc4n(c21)-c1ccccc1-3]

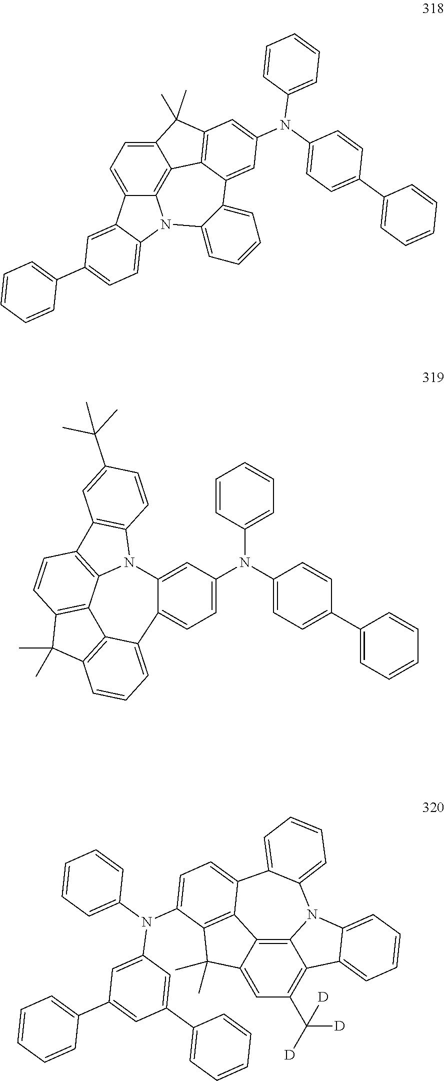 [SMILES: CC1(C)c2cc(N(c3ccccc3)c3ccc(-c4ccccc4)cc3)cc3c2-c2c1ccc1c4cc(-c5ccccc5)ccc4n(c21)-c1ccccc1-3 ; CC(C)(C)c1ccc2c(c1)c1ccc3c4c1n2-c1cc(N(c2ccccc2)c2ccc(-c5ccccc5)cc2)ccc1-c1cccc(c1-4)C3(C)C ; [2H]C([2H])([2H])c1cc2c3c4c1c1ccccc1n4-c1ccccc1-c1ccc(N(c4ccccc4)c4cc(-c5ccccc5)cc(-c5ccccc5)c4)c(c1-3)C2(C)C]

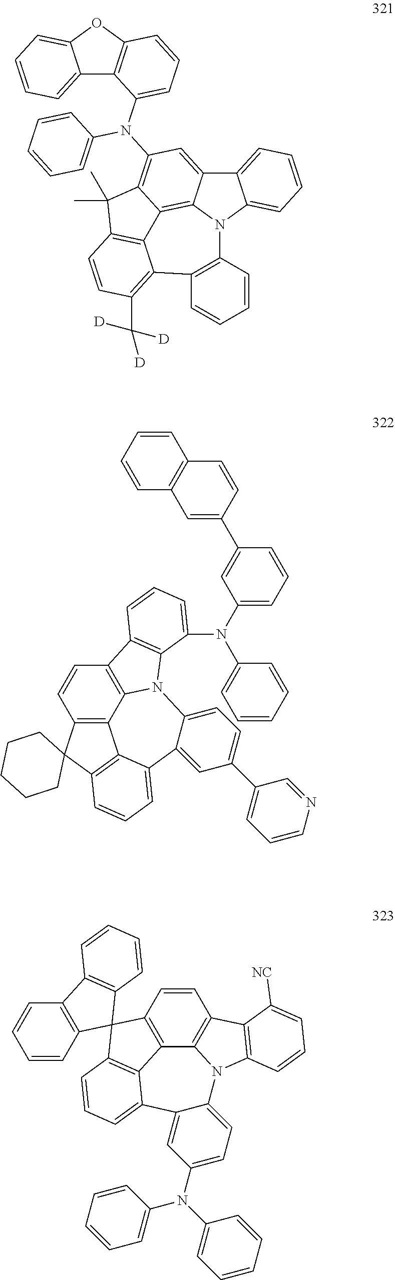 [SMILES: [2H]C([2H])([2H])c1ccc2c3c1-c1ccccc1-n1c4ccccc4c4cc(N(c5ccccc5)c5cccc6oc7ccccc7c56)c(c-3c41)C2(C)C ; c1ccc(N(c2cccc(-c3ccc4ccccc4c3)c2)c2cccc3c4ccc5c6c4n(c23)-c2ccc(-c3cccnc3)cc2-c2cccc(c2-6)C52CCCCC2)cc1 ; N#Cc1cccc2c1c1ccc3c4c1n2-c1ccc(N(c2ccccc2)c2ccccc2)cc1-c1cccc(c1-4)C31c2ccccc2-c2ccccc21]

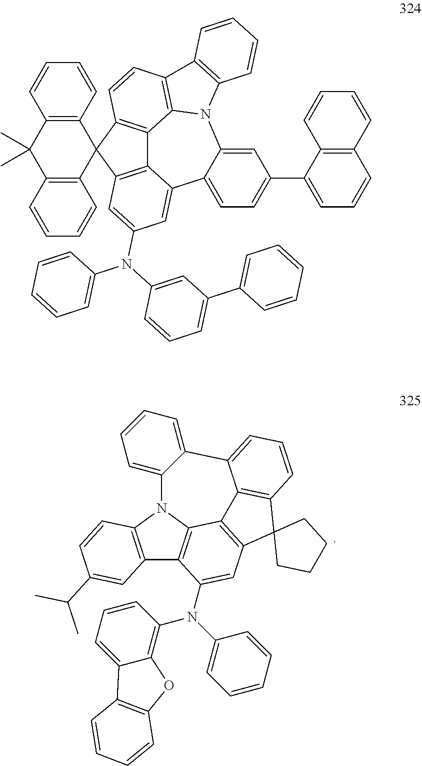 [SMILES: CC1(C)c2ccccc2C2(c3ccccc31)c1cc(N(c3ccccc3)c3cccc(-c4ccccc4)c3)cc3c1-c1c2ccc2c4ccccc4n(c12)-c1cc(-c2cccc4ccccc24)ccc1-3 ; CC(C)c1ccc2c(c1)c1c(N(c3ccccc3)c3cccc4c3oc3ccccc34)cc3c4c1n2-c1ccccc1-c1cccc(c1-4)C31CCCC1]

The present disclosure also provides an electronic element, including: an anode and a cathode that are arranged oppositely, and at least one functional layer between the anode and the cathode, wherein the functional layer includes the organic compound of the present disclosure.

Optionally, the functional layer may include an HTL and/or an EBL, and the EBL or HTL may include the organic compound.

Optionally, the electronic element of the present disclosure may be an OLED or a solar cell, and further optionally, the OLED may be a red light-emitting OLED or a green light-emitting OLED.

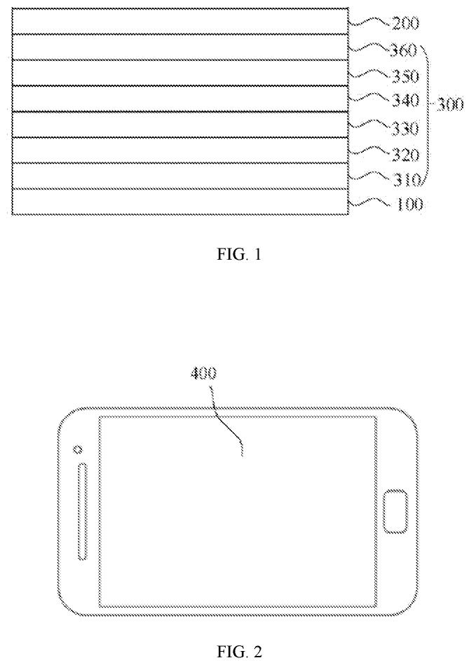

In a specific embodiment of the present disclosure, as shown in FIG. 1, the OLED may include an anode 100, a cathode 200, and at least one functional layer 300 between the anode and the cathode; the functional layer 300 may include an HIL 310, an HTL 320, an EBL 330, an organic electroluminescent layer 340, an ETL 350, and an EIL 360; and the HIL 310, the HTL 320, the EBL 330, the organic electroluminescent layer 340, the ETL 350, and the EIL 360 may be sequentially formed on the anode 100. The HTL 320 and/or the EBL 330 may include the organic compound of the present disclosure.

Optionally, the anode 100 may be preferably made of a material with a large work function that facilitates the injection of holes into the functional layer. Specific examples of the anode material may include, but are not limited to: metals such as nickel, platinum, vanadium, chromium, copper, zinc, and gold or alloys thereof; metal oxides such as zinc oxide, indium oxide, indium tin oxide (ITO), and indium zinc oxide (IZO); a combination of a metal and an oxide such as ZnO: Al or SnO₂: Sb; or conductive polymers such as poly(3-methylthiophene), poly[3,4-(ethylene-1,2-dioxy)thiophene] (PEDT), polypyrrole (PPy), and polyaniline (PANI). Preferably, a transparent electrode with ITO may be adopted as the anode.

Optionally, the HTL 320 may include one or more hole transport materials, and the hole transport materials may be carbazole polymers, carbazole-linked triarylamine compounds, or other compounds, which are not particularly limited in the present disclosure. In one embodiment of the present disclosure, the HTL 320 includes the compound N,N′-di-1-naphthalenyl-N,N′-diphenyl-[1,1′-biphenyl]-4,4′-diamine (NPB). In another embodiment of the present disclosure, the HTL 320 includes the organic compound of the present disclosure.

Optionally, the EBL 330 may be arranged to block electrons transmitted from the organic electroluminescent layer 340, thereby ensuring that an electron and a hole can be efficiently recombined in the organic electroluminescent layer 340; the EBL 330 can also block excitons diffused from the organic electroluminescent layer 340 to reduce the triplet-state quenching of the excitons, thereby ensuring the light-emitting efficiency of the OLED; and the EBL 330 can effectively block the transmission and diffusion of electrons and excitons from the organic electroluminescent layer 340 to the anode 100. Preferably, the EBL 330 includes the organic compound of the present disclosure.

The organic electroluminescent layer 340 may be prepared from a single light-emitting material, or may include a host material and a dopant material. Optionally, the organic electroluminescent layer 340 may include a host material and a dopant material, wherein holes and electrons injected into the organic electroluminescent layer 340 can be recombined in the organic electroluminescent layer 340 to form excitons, the excitons transfer energy to the host material, and then the host material transfers energy to the dopant material, such that the dopant material can emit light.

The host material of the organic electroluminescent layer 340 may be a metal chelate compound, a bisstyryl derivative, an aromatic amine derivative, a dibenzofuran derivative, or the like, which is not particularly limited in the present disclosure. For example, the host material of the organic electroluminescent layer 340 is 4,4′-N,N′-dicarbazole-biphenyl (CBP).

The dopant material of the organic electroluminescent layer 340 may be a compound with a condensed aryl ring or a derivative thereof, a compound with a heteroaryl ring or a derivative thereof, an aromatic amine derivative, or the like, which is not particularly limited in the present disclosure. For example, the dopant material of the organic electroluminescent layer 340 is Ir(piq)₂(acac).

The ETL 350 may have a single-layer structure or a multi-layer structure, which may include one or more electron transport materials. The electron transport materials may be benzimidazole derivatives, oxadiazole derivatives, quinoxaline derivatives, or other electron transport materials. From the perspective of molecular design, the organic compound of the present disclosure has an electron-deficient large conjugated planar structure, and has advantages such as asymmetric structure and large steric hindrance, which can reduce the intermolecular cohesion and crystallization tendency, thereby increasing the electron transport rate. For example, the ETL 350 includes ET-06 and LiQ.

Optionally, the cathode 200 may be made of a material with a small work function that facilitates the injection of electrons into the functional layer. Specific examples of the cathode material may include, but are not limited to: metals such as magnesium, calcium, sodium, potassium, titanium, indium, yttrium, lithium, gadolinium, aluminum, silver, tin and lead or alloys thereof; or multi-layer materials such as LiF/Al, Liq/Al, LiO₂/Al, LiF/Ca, LiF/Al, and BaF₂/Ca. Preferably, a metal electrode with silver and magnesium may be adopted as the cathode.

Optionally, an HIL 310 may be further arranged between the anode 100 and the HTL 320 to enhance the ability to inject holes into the HTL 320. The HIL 310 can be made of a benzidine derivative, a starburst arylamine compound, a phthalocyanine derivative, or another material, which is not particularly limited in the present disclosure. For example, the HIL 310 includes F4-TCNQ.

Optionally, an EIL 360 may be further arranged between the cathode 200 and the ETL 350 to enhance the ability to inject electrons into the ETL 350. The EIL 360 may include an inorganic material such as an alkali metal sulfide and an alkali metal halide, or may include a complex of an alkali metal and an organic substance. For example, the EIL 360 includes ytterbium (Yb).

The present disclosure also provides an electronic device including the electronic element of the present disclosure.

For example, as shown in FIG. 2, the present disclosure provides an electronic device 400, and the electronic device 400 includes the OLED. The electronic device may be a display element, a lighting element, an optical communication element, or another electronic device, including but not limited to computer screen, mobile phone screen, television set, electronic paper, emergency light, and optical module.

The present disclosure will be described in detail below with reference to examples, but the following description is provided to explain the present disclosure rather than limit the scope of the present disclosure in any way.

### Synthesis Examples

Those skilled in the art will recognize that the chemical reactions described in the present disclosure can be used to appropriately prepare many other compounds of the present disclosure, and other methods for preparing the compounds of the present disclosure are considered to be within the scope of the present disclosure. For example, the synthesis of non-illustrative compounds according to the present disclosure can be successfully completed by those skilled in the art through modified methods, such as appropriately protecting interfering groups, using other known reagents in addition to those described in the present disclosure, or conventionally modifying reaction conditions.

In the synthesis examples described below, unless otherwise stated, all temperatures are in degrees Celsius (° C.). Some reagents are purchased from commodity suppliers such as Aldrich Chemical Company, Arco Chemical Company, and Alfa Chemical Company, and some intermediates that cannot be directly purchased are prepared from commercially-available raw materials through simple reactions, which are used without further purification unless otherwise stated. The remaining conventional reagents are purchased from Shantou Xilong Chemical Co., Ltd., Guangdong Guanghua Chemical Reagent Factory Co., Ltd., Guangzhou Chemical Reagent Factory, Tianjin Haoyuyu Chemical Co., Ltd., Tianjin Fuchen Chemical Reagent Factory, Wuhan Xinhuayuan Technology Development Co., Ltd., Qingdao Tenglong Chemical Reagent Co., Ltd., and Qingdao Haiyang Chemical Co., Ltd. Anhydrous solvents such as anhydrous tetrahydrofuran (THF), dioxane, toluene, and diethyl ether are obtained through drying with metal sodium under reflux. The reactions in the synthesis examples are generally conducted under a positive pressure of nitrogen or argon or in a drying tube with an anhydrous solvent (unless otherwise stated); and during the reactions, a reaction flask is plugged with a suitable rubber plug, a substrate is injected into the reaction flask through a syringe, and all glass wares involved are dry.

During purification, a chromatographic column is a silica gel column, and silica gel (100 to 200 mesh) is purchased from Qingdao Haiyang Chemical Co., Ltd.

In each synthesis example, low-resolution mass spectrometry (MS) data are obtained under the following conditions: Agilent 6120 quadrupole HPLC-M (column model: Zorbax SB-C18, 2.1×30 mm, 3.5 μm, 6 min, flow rate: 0.6 mL/min; and mobile phase: a proportion of (acetonitrile with 0.1% formic acid) in (water with 0.1% formic acid): 5% to 95%, electrospray ionization (ESI), and ultraviolet (UV) detection at 210 nm/254 nm.

¹H nuclear magnetic resonance (NMR) spectroscopy: Through a Bruker 400 MHz NMR spectrometer, the NMR spectroscopy is conducted at room temperature with CDCl₃ (in ppm) as a solvent and tetramethylsilane (TMS) (0 ppm) as a reference standard. When multiplets appear, the following abbreviations will be adopted: s: singlet, d: doublet, t: triplet, and m: multiplet.

A target compound is tested using Agilent 1260 pre-HPLC or Calesep pump 250 pre-HPLC (column model: NOVASEP 50/80 mm DAC): UV detection at 210 nm/254 nm.

### 1. Synthesis of an Intermediate SY Z-X

### (1) Synthesis of an Intermediate SA 3-1

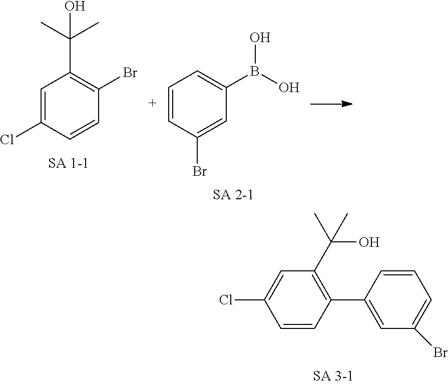

Nitrogen was introduced into a three-necked flask equipped with a mechanical stirrer, a thermometer, and a spherical condenser at 0.100 L/min to allow nitrogen replacement for 15 min, then a reactant SA 1-1 (211.54 g, 847.84 mmol), a reactant SA 2-1 (170.25 g, 847.84 mmol), THF (1,272 mL), and H₂O (424 mL) were added, and a resulting mixture was heated to reflux and stirred until the resulting solution was clear. Tetrabutylammonium bromide (TBAB) (5.47 g, 16.96 mmol), tetrakis(triphenylphosphine)palladium (9.80 g, 8.48 mmol), and potassium carbonate (175.77 g, 1,271.76 mmol) were added, a resulting mixture was stirred until the resulting solution was clear, and then heated to reflux and stirred for 24 h. After the reaction was completed, the resulting reaction mixture was cooled to room temperature; dichloromethane (DCM) was added for extraction, the separated organic phase was washed with water until neutral and dried with anhydrous magnesium sulfate, filtered, and a filtrate was concentrated in vacuum to obtain a crude product; and the crude product was purified by silica gel column chromatography to obtain the intermediate SA 3-1 (196 g, yield: 71.3%).

The intermediates SA 3-X (each X was an integer of 2 to 5) shown in table 1 below were each synthesized with reference to the synthesis method of the intermediate SA 3-1, wherein the reactants SA 1-X (each X was an integer of 2 to 4) were used instead of the reactant SA 1-1 and the reactants SA 2-X (each X was an integer of 1 to 3) were used instead of the reactant SA 2-1.

TABLE 1

| Reactant SA 1-X | Reactant SA 2-X | Intermediate SA 3-X | Mass (g) | Yield (%) |
|---|---|---|---|---|
| 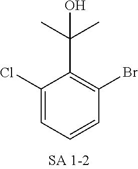 | 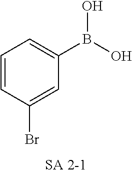 | 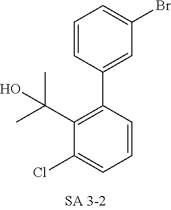 [SMILES: CC(C)(O)c1c(Cl)cccc1-c1cccc(Br)c1] | 187.72 | 68 |
| 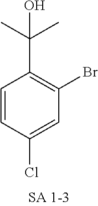 | | 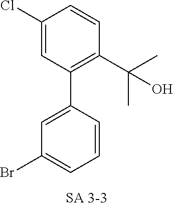 [SMILES: CC(C)(O)c1ccc(Cl)cc1-c1cccc(Br)c1] | 201.52 | 73 |
| 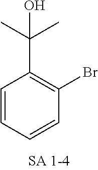 | 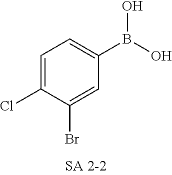 [SMILES: OB(O)c1ccc(Cl)c(Br)c1] | 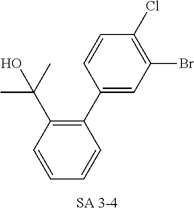 [SMILES: CC(C)(O)c1ccccc1-c1ccc(Cl)c(Br)c1] | 187.96 | 67 |
| | 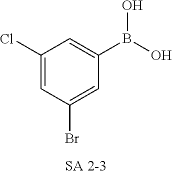 [SMILES: OB(O)c1cc(Cl)cc(Br)c1] | 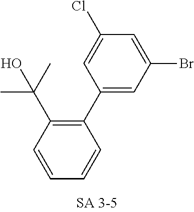 [SMILES: CC(C)(O)c1ccccc1-c1cc(Cl)cc(Br)c1] | 182.20 | 66 |

### (2) Synthesis of an Intermediate SA 4-1

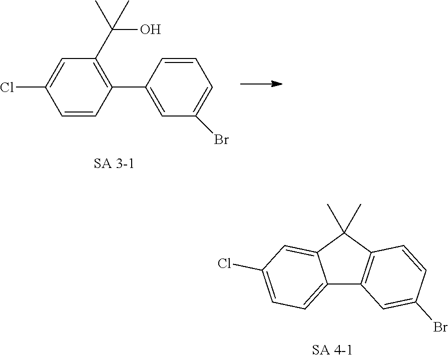 [SMILES: CC(C)(O)c1cc(Cl)ccc1-c1cccc(Br)c1 ; CC1(C)c2ccc(Br)cc2-c2ccc(Cl)cc21]

Nitrogen was introduced into a three-necked flask equipped with a mechanical stirrer, a thermometer, and a spherical condenser at 0.100 L/min to allow nitrogen replacement for 15 min, then the intermediate SA 3-1 (190 g, 585.2 mmol), acetic acid (900 mL), and phosphoric acid (50 mL) were added, and a resulting mixture was heated to 50° C. and stirred until a resulting solution was clear; then reaction solution was stirred for 4 h. After the reaction was completed, the resulting reaction solution was cooled to room temperature; a NaOH aqueous solution was added for neutralization to pH=7, then ethyl acetate was added for extraction, the combined organic phases were dried with anhydrous magnesium sulfate, and filtered, and a resulting filtrate was concentrated in vacuum to obtain a crude product; and the crude product was purified by silica gel column chromatography to obtain the intermediate SA 4-1 (122.56 g, yield: 68.2%).

The intermediates SA 4-X (each X was an integer of 2 to 5) shown in table 2 below were each synthesized with reference to the synthesis method of the intermediate SA 4-1, wherein the intermediates SA 3-X (each X was an integer of 2 to 5) were used instead of the reactant SA 3-1.

TABLE 2

| Intermediate SA 3-X | Intermediate SA 4-X | Mass (g) | Yield (%) |
|---|---|---|---|
| 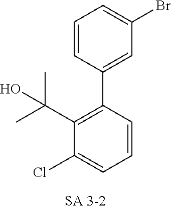 [SMILES: CC(C)(O)c1c(Cl)cccc1-c1cccc(Br)c1] | 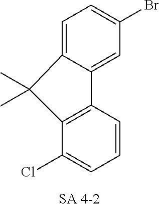 [SMILES: CC1(C)c2ccc(Br)cc2-c2cccc(Cl)c21] | 118.96 | 66 |
| 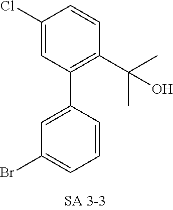 [SMILES: CC(C)(O)c1ccc(Cl)cc1-c1cccc(Br)c1] | 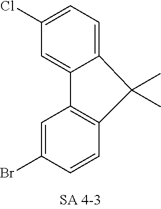 [SMILES: CC1(C)c2ccc(Cl)cc2-c2cc(Br)ccc21] | 122.56 | 68 |
| 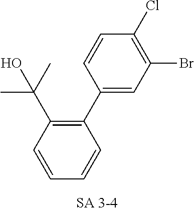 [SMILES: CC(C)(O)c1ccccc1-c1ccc(Cl)c(Br)c1] | 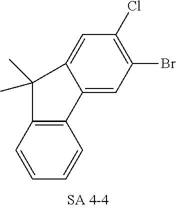 [SMILES: CC1(C)c2ccccc2-c2cc(Br)c(Cl)cc21] | 115.35 | 64 |
| 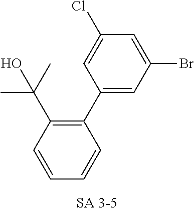 [SMILES: CC(C)(O)c1ccccc1-c1cc(Cl)cc(Br)c1] | 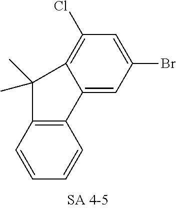 [SMILES: CC1(C)c2ccccc2-c2cc(Br)cc(Cl)c21] | 113.40 | 63 |

### (3) Synthesis of an Intermediate SB 2-1

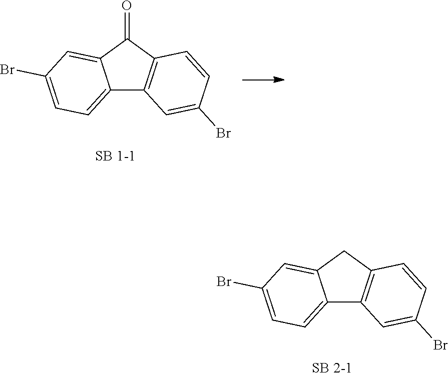 [SMILES: O=C1c2cc(Br)ccc2-c2cc(Br)ccc21 ; Brc1ccc2c(c1)Cc1ccc(Br)cc1-2]

Raney nickel (6 g), hydrazine hydrate (83 mL, 1,716 mmol), a reactant SB 1-1 (145 g, 429 mmol), toluene (870 mL), and ethanol (290 mL) were added to a three-necked flask, a resulting mixture was quickly stirred and heated to reflux, and stirred for 2 h. After the reaction was completed, the reaction solution was concentrated in vacuum to obtain a residue, and the residue was purified by silica gel column chromatography to obtain the intermediate SB 2-1 (104.25 g, yield: 75.2%).

The intermediates SB 2-X (each X was an integer of 2 to 5) shown in table 3 below were each synthesized with reference to the synthesis method of the intermediate SB 2-1, wherein the reactants SB 1-X (each X was an integer of 2 to 5) were used instead of the reactant SB 1-1.

TABLE 3

| Reactant SB 1-X | Intermediate SB 2-X | Yield (%) |
|---|---|---|
| 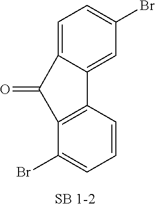 [SMILES: O=C1c2ccc(Br)cc2-c2cccc(Br)c21] | 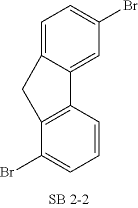 [SMILES: Brc1ccc2c(c1)-c1cccc(Br)c1C2] | 76.8 |
| 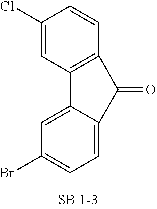 [SMILES: O=C1c2ccc(Cl)cc2-c2cc(Br)ccc21] | 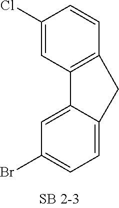 [SMILES: Clc1ccc2c(c1)-c1cc(Br)ccc1C2] | 78.1 |
| 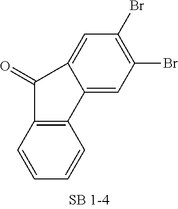 [SMILES: O=C1c2ccccc2-c2cc(Br)c(Br)cc21] | 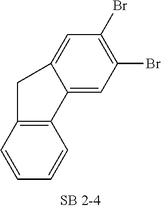 [SMILES: Brc1cc2c(cc1Br)-c1ccccc1C2] | 71.5 |
| 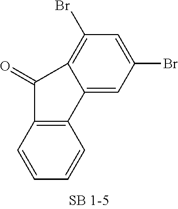 [SMILES: O=C1c2ccccc2-c2cc(Br)cc(Br)c21] | 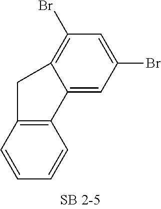 [SMILES: Brc1cc(Br)c2c(c1)-c1ccccc1C2] | 73.4 |

### (4) Synthesis of an Intermediate SB 4-1

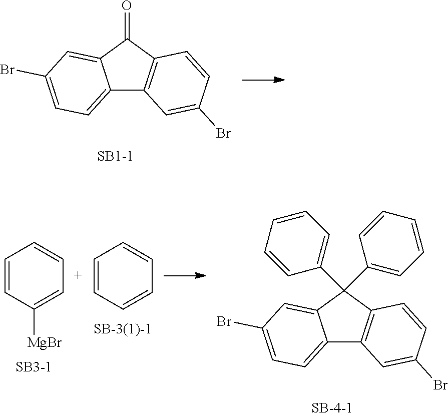

The SB 1-1 (109 g, 322.48 mmol) and anhydrous THF (545 mL) were added to a three-necked flask, a resulting mixture was cooled to −10° C., then the SB 3-1 (61.29 g, 338.60 mmol) was added, and a resulting mixture was continuously stirred until it was warmed to room temperature; then NH₄Cl (500 mL) was added for quenching, ethyl acetate was added to a resulting reaction mixture for extraction. The combined organic phases were washed with water, dried with anhydrous sodium sulfate, and concentrated in vacuum to obtain a residue. The residue was purified by recrystallization with toluene and n-heptane. A solid obtained after the recrystallization was added to a three-necked flask with DCM (200 mL), then the SB-3(1)-1 (dissolved in benzene, 25.19 g, 322.48 mmol) was added, and a resulting mixture was heated to 50° C.; then trifluoromethanesulfonic acid (80 mL) was added dropwise to allow a reaction for 30 min, a resulting reaction system was washed with water, and a resulting organic phase was separated, dried with anhydrous sodium sulfate, and concentrated in vacuum to obtain a residue; and the residue was purified by a silica gel column and eluted with n-heptane/ethyl acetate to obtain the intermediate SB 4-1 (112.10 g, yield: 73.0%).

The intermediates SB 4-X (each X was an integer of 2 to 7) shown in table 4 below were each synthesized with reference to the synthesis method of the intermediate SB 4-1, wherein the reactants SB 1-X (each X was an integer of 2 to 5) were used instead of the reactant SB1-1, the SB-3(1)-X were used instead of the reactant benzene, and the reactants SB 3-X (each X was an integer of 1 to 6) were used instead of the reactant SB 3-1.

TABLE 4

| Reactant SB 1-X | Reactant SB 3-X | SB-3(1)-X | Intermediate SB 4-X | Yield (%) |
|---|---|---|---|---|
| 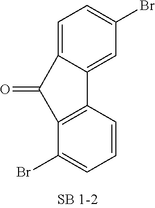 [SMILES: O=C1c2ccc(Br)cc2-c2cccc(Br)c21] | 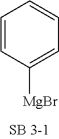 | 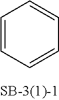 | 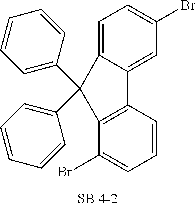 [SMILES: Brc1ccc2c(c1)-c1cccc(Br)c1C2(c1ccccc1)c1ccccc1] | 76.8 |
| 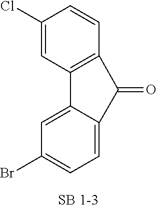 [SMILES: O=C1c2ccc(Cl)cc2-c2cc(Br)ccc21] | 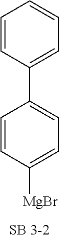 | 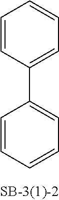 [SMILES: c1ccc(-c2ccccc2)cc1] | 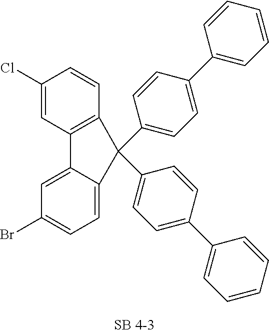 [SMILES: Clc1ccc2c(c1)-c1cc(Br)ccc1C2(c1ccc(-c2ccccc2)cc1)c1ccc(-c2ccccc2)cc1] | 71.2 |
| 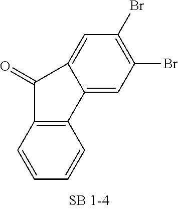 [SMILES: O=C1c2ccccc2-c2cc(Br)c(Br)cc21] | 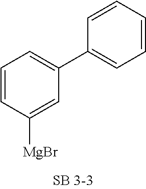 | 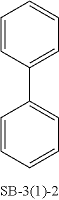 [SMILES: c1ccc(-c2ccccc2)cc1] | 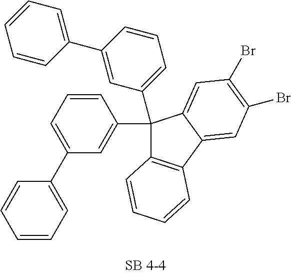 [SMILES: Brc1cc2c(cc1Br)C(c1cccc(-c3ccccc3)c1)(c1cccc(-c3ccccc3)c1)c1ccccc1-2] | 68.6 |
| 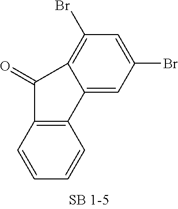 [SMILES: O=C1c2ccccc2-c2cc(Br)cc(Br)c21] | 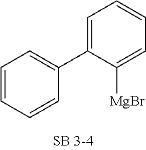 | 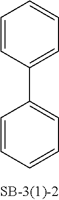 [SMILES: c1ccc(-c2ccccc2)cc1] | 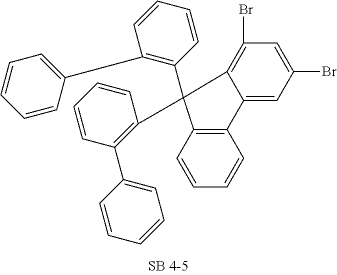 [SMILES: Brc1cc(Br)c2c(c1)-c1ccccc1C2(c1ccccc1-c1ccccc1)c1ccccc1-c1ccccc1] | 67.8 |
| 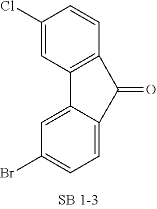 [SMILES: O=C1c2ccc(Cl)cc2-c2cc(Br)ccc21] | 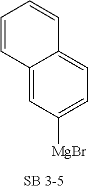 | 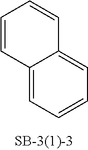 | 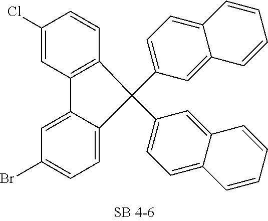 [SMILES: Clc1ccc2c(c1)-c1cc(Br)ccc1C2(c1ccc2ccccc2c1)c1ccc2ccccc2c1] | 70.3 |
| 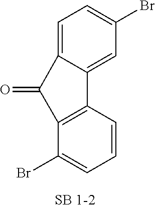 [SMILES: O=C1c2ccc(Br)cc2-c2cccc(Br)c21] | 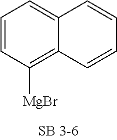 | 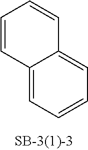 | 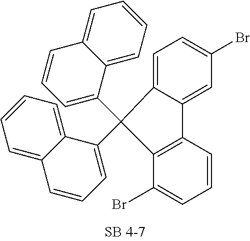 [SMILES: Brc1ccc2c(c1)-c1cccc(Br)c1C2(c1cccc2ccccc12)c1cccc2ccccc12] | 68.5 |

### (5) Synthesis of an Intermediate SB 6-1

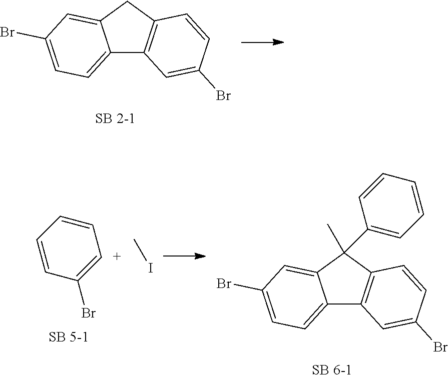

Under a nitrogen atmosphere, the intermediate SB 2-1 (107 g, 330.25 mmol), the reactant SB 5-1 (38.89 g, 247.69 mmol), dioxane, potassium tert-butoxide (92.64 g, 825.63 mmol), and Pd₂(dba)₃ (6.05 g, 6.605 mmol) were added to a three-necked flask, a resulting mixture was stirred and heated to 120° C., and stirred for 12 h; iodomethane (46.88 g, 330.25 mmol) was added, and a resulting mixture was stirred at room temperature for 6 h. After the reaction was completed, a resulting reaction system was washed with water until neutral and passed through a silica gel column, eluted with a mixture of petroleum ether (PE) and ethyl acetate (in a volume ratio of 10:1) for purification to obtain the intermediate SB 6-1 (112.11 g, yield: 82.0%).

The intermediates SB 6-X (each X was an integer of 2 to 5) shown in table 5 below were each synthesized with reference to the synthesis method of the intermediate SB-6-1, wherein the reactants SB 2-X (each X was an integer of 2 to 5) were used instead of the intermediate SB 2-1 and the reactants SB 5-X (each X was an integer of 2 to 5) were used instead of the reactant SB 5-1.

TABLE 5

| Intermediate SB 2-X | Reactant SB 5-X | Intermediate SB 6-X | Yield (%) |
|---|---|---|---|
| 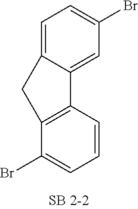 [SMILES: Brc1ccc2c(c1)-c1cccc(Br)c1C2] | 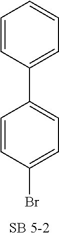 [SMILES: Brc1ccc(-c2ccccc2)cc1] | 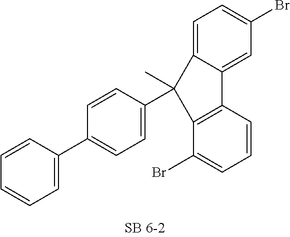 [SMILES: CC1(c2ccc(-c3ccccc3)cc2)c2ccc(Br)cc2-c2cccc(Br)c21] | 78.5 |
| 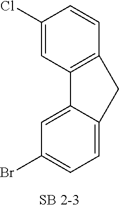 [SMILES: Clc1ccc2c(c1)-c1cc(Br)ccc1C2] | 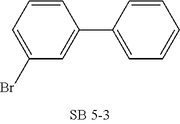 [SMILES: Brc1cccc(-c2ccccc2)c1] | 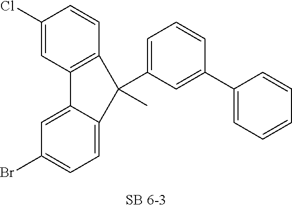 [SMILES: CC1(c2cccc(-c3ccccc3)c2)c2ccc(Cl)cc2-c2cc(Br)ccc21] | 76.8 |
| 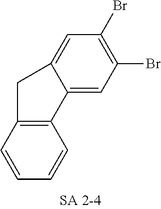 [SMILES: Brc1cc2c(cc1Br)-c1ccccc1C2] | 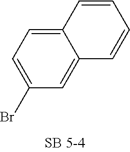 [SMILES: Brc1ccc2ccccc2c1] | 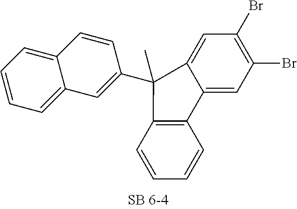 [SMILES: CC1(c2ccc3ccccc3c2)c2ccccc2-c2cc(Br)c(Br)cc21] | 80.2 |
| 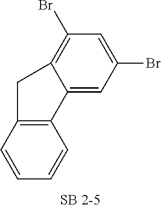 [SMILES: Brc1cc(Br)c2c(c1)-c1ccccc1C2] | 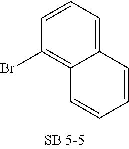 [SMILES: Brc1cccc2ccccc12] | 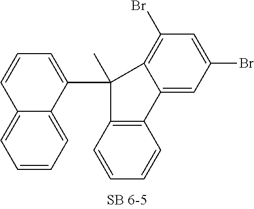 [SMILES: CC1(c2cccc3ccccc23)c2ccccc2-c2cc(Br)cc(Br)c21] | 76.4 |

### (6) Synthesis of an Intermediate SB 8-1

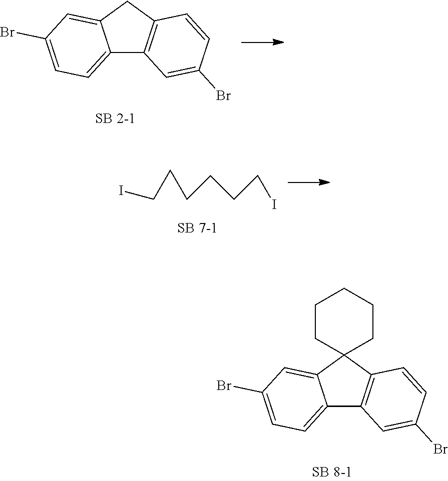 [SMILES: Brc1ccc2c(c1)Cc1ccc(Br)cc1-2 ; ICCCCCCI ; Brc1ccc2c(c1)-c1ccc(Br)cc1C21CCCCC1]

The intermediate SB 2-1 (121 g, 373.46 mmol) was dissolved in anhydrous dimethylsulfoxide (DMSO) (605 mL) in a three-necked flask, then sodium tert-butoxide (53.83 g, 560.19 mmol) was added at room temperature, and a resulting mixture was stirred and heated to 65° C. A reactant SB 7-1 (252.44 g, 746.92 mmol) was dissolved in anhydrous DMSO and then added dropwise to the above three-necked flask, and after the dropwise addition, a resulting mixture was kept at 65° C. for 30 min. After a reaction was completed, 300 mL of a NH₄OH aqueous solution was added, a resulting mixture was stirred for 20 min and filtered, and a filter cake was washed with methanol and water to obtain a crude product; and the crude product was purified by silica gel column chromatography to obtain the intermediate SB 8-1 (111.30 g, yield: 76%).

The intermediates SB 8-X (each X was an integer of 3 to 5) shown in table 6 below were each synthesized with reference to the synthesis method of the intermediate SB 8-1, wherein the intermediates SB 2-X (each X was an integer of 3 to 5) were used instead of the intermediate SB 2-1 and the reactants SB 7-X (each X was 1 or 2) were used instead of the reactant SB 7-1.

TABLE 6

| Intermediate SB 2-X | Reactant SB 7-X | Intermediate SB 8-X | Yield (%) |
|---|---|---|---|
| 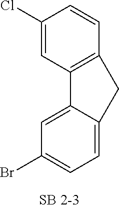 [SMILES: Clc1ccc2c(c1)-c1cc(Br)ccc1C2] | 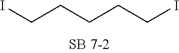 | 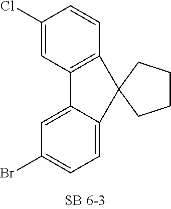 [SMILES: Clc1ccc2c(c1)-c1cc(Br)ccc1C21CCCC1] | 77.2 |
| 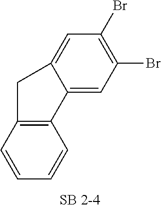 [SMILES: Brc1cc2c(cc1Br)-c1ccccc1C2] | 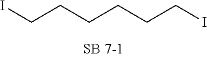 | 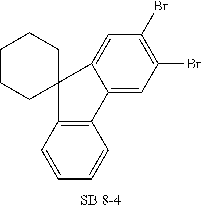 [SMILES: Brc1cc2c(cc1Br)C1(CCCCC1)c1ccccc1-2] | 75.8 |
| 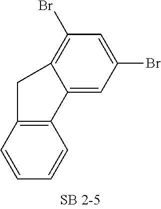 [SMILES: Brc1cc(Br)c2c(c1)-c1ccccc1C2] | 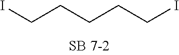 | 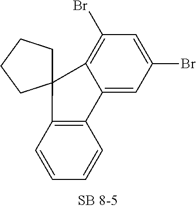 [SMILES: Brc1cc(Br)c2c(c1)-c1ccccc1C21CCCC1] | 76.1 |

### (7) Synthesis of an Intermediate SB 9-1

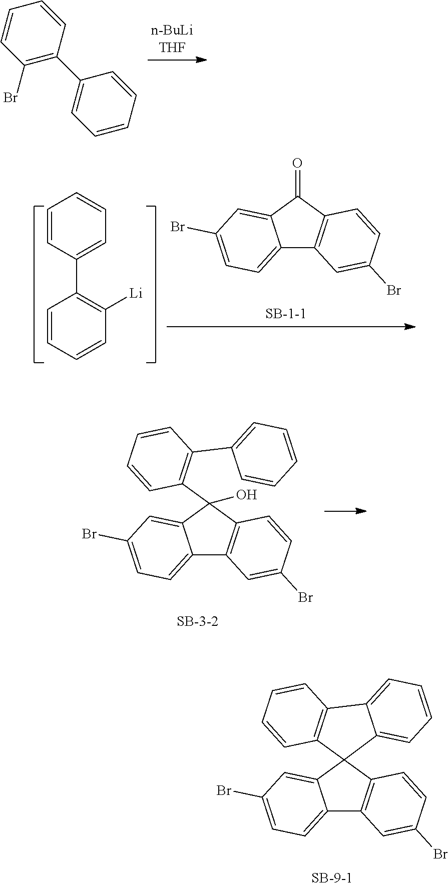

Under a fully-dry condition and a nitrogen atmosphere, 2-bromo-1,1-biphenyl (105.5 g, 452.58 mmol) and 600 mL of anhydrous THF were added to a 1 L four-necked flask, a resulting mixture was stirred for dissolution and then cooled with liquid nitrogen to −78° C. or lower, 120 mL of a solution of n-BuLi in n-hexane (452.58 mmol) was slowly added dropwise, and after the dropwise addition, a resulting mixture was stirred at −78° C. for 1 h; then the SB-1-1 (152.97 g, 452.58 mmol) solid was added in batches at this temperature, and a resulting mixture was kept at −78° C. for 1 h and then stirred at room temperature for 12 h. After a reaction was completed, 8 mL of a hydrochloric acid solution was added dropwise for quenching, ethyl acetate was added for extraction, and the separated organic phase was washed with saturated brine, dried with anhydrous sodium sulfate, and concentrated in vacuum to obtain an intermediate SB-3-2. The intermediate SB-3-2 was directly added to a 2 L dry three-necked flask without further purification, then 1335 mL of acetic acid and 20 g of hydrochloric acid with a mass fraction of 36% were added, a resulting mixture was heated to reflux and stirred for 3 h, and then the reaction was stopped; a resulting reaction system was cooled to room temperature, filtered, and washed twice with water, and a resulting organic phase was separated, dried with anhydrous sodium sulfate, and concentrated in vacuum to obtain a residue; and the resulting residue was purified by silica gel column chromatography to obtain the intermediate SB-9-1 (123.40 g, yield: 57.5%).

The intermediates SB 9-X (each X was an integer of 2 to 4) shown in table 7 below were each synthesized with reference to the synthesis method of the intermediate SB 9-1, wherein the reactants SB 1-X (each X was an integer of 2 to 4) were used instead of the reactant SB 1-1.

TABLE 7

| Reactant SB 1-X | Intermediate SB 9-X | Yield (%) |
|---|---|---|
| 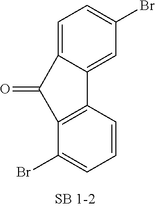 [SMILES: O=C1c2ccc(Br)cc2-c2cccc(Br)c21] | 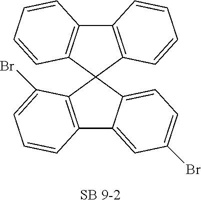 [SMILES: Brc1ccc2c(c1)-c1cccc(Br)c1C21c2ccccc2-c2ccccc21] | 56.1 |
| 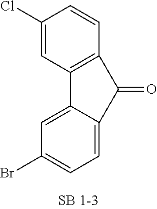 [SMILES: O=C1c2ccc(Cl)cc2-c2cc(Br)ccc21] | 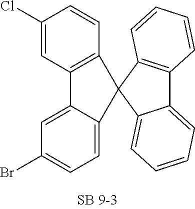 [SMILES: Clc1ccc2c(c1)-c1cc(Br)ccc1C21c2ccccc2-c2ccccc21] | 57.2 |
| 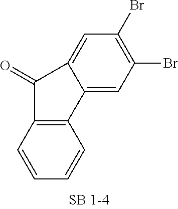 [SMILES: O=C1c2ccccc2-c2cc(Br)c(Br)cc21] | 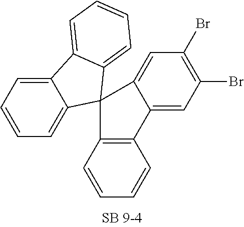 [SMILES: Brc1cc2c(cc1Br)C1(c3ccccc3-c3ccccc31)c1ccccc1-2] | 56.8 |

### (8) Synthesis of an Intermediate SC 3-1

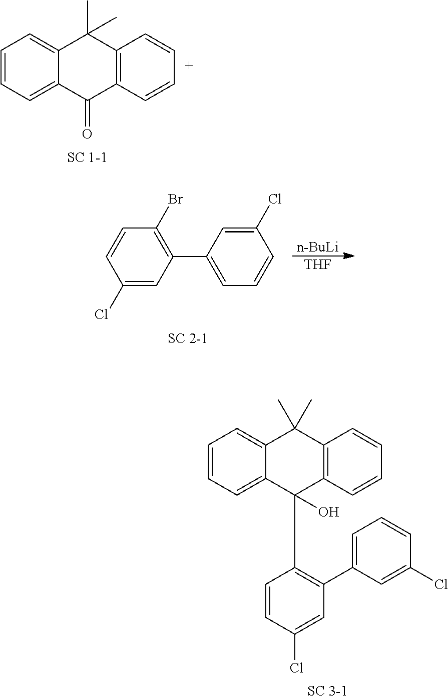

Under a nitrogen atmosphere, the reactant SC 1-1 (128.3 g, 577.17 mmol) and THF (768 mL) were added to a three-necked flask, and a resulting mixture was thoroughly stirred and cooled to −78° C.; then an n-butyllithium solution (92 g, 1,442.8 mmol) was added dropwise, and after the dropwise addition, a resulting mixture was stirred at −78° C. for 1 h; then the reactant SC 2-1 (168.26 g, 577.17 mmol) was diluted with THF (336 mL) in a dilution ratio of 1:2 and then added dropwise, and after the dropwise addition, a resulting mixture was stirred at −78° C. for another 1 h, then naturally warmed to 25° C., and stirred for 12 h. After a reaction was completed, a resulting reaction solution was poured into water (1,000 mL) and stirred for 10 min, then extracted twice with DCM (800 mL), and the combined organic phases were dried with anhydrous magnesium sulfate, and concentrated in vacuum to obtain a residue; and the residue was filtered by a silica gel funnel, and then a filtrate was concentrated in vacuum to obtain the intermediate SC 3-1 (166.9 g, yield: 65%).

The intermediate SC 3-2 shown in table 8 below was synthesized with reference to the synthesis method of the intermediate SC 3-1, wherein the reactant SC 2-2 was used instead of the reactant SC 2-1.

TABLE 8

| Reactant SC 1-1 | Reactant SC 2-2 | Intermediate SC 3-2 | Yield (%) |
|---|---|---|---|
| 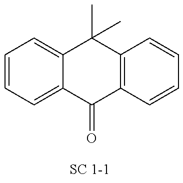 [SMILES: CC1(C)c2ccccc2C(=O)c2ccccc21] | 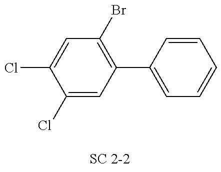 [SMILES: Clc1cc(Br)c(-c2ccccc2)cc1Cl] | 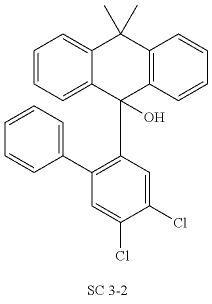 [SMILES: CC1(C)c2ccccc2C(O)(c2cc(Cl)c(Cl)cc2-c2ccccc2)c2ccccc21] | 61.5 |

### (9) Synthesis of an Intermediate SC 4-1

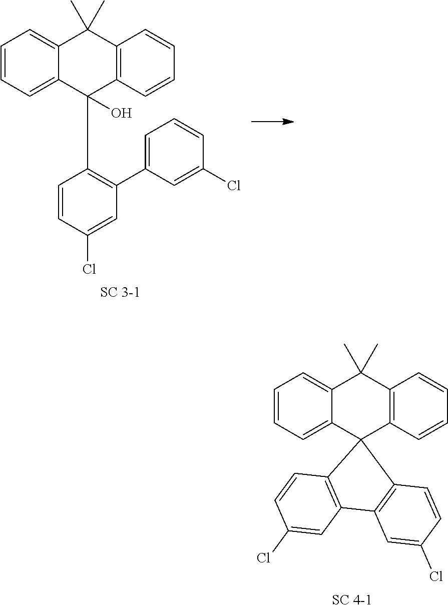 [SMILES: CC1(C)c2ccccc2C(O)(c2ccc(Cl)cc2-c2cccc(Cl)c2)c2ccccc21 ; CC1(C)c2ccccc2C2(c3ccc(Cl)cc3-c3cc(Cl)ccc32)c2ccccc21]

The intermediate SC 3-1 (153 g, 343.53 mmol) and trifluoroacetic acid (TFA) (459 mL) were added to a single-necked flask, and a resulting mixture was stirred and then gradually heated at 80° C. to reflux and stirred for 11 h. After the reaction was completed, a resulting reaction solution was poured into water, a resulting mixture was stirred for 30 min and filtered, and a filter residue was rinsed with water and ethanol successively to obtain a crude product; and the crude product was purified by recrystallization with DCM: n-heptane=1:2 (v/v) to obtain the intermediate SC 4-1 (111.58 g, yield: 76%).

The intermediate SC 4-2 shown in table 9 below was synthesized with reference to the synthesis method of the intermediate SC 4-1, wherein the intermediate SC 3-2 was used instead of the intermediate SC 3-1.

TABLE 9

| Intermediate SC 3-2 | Intermediate SC 4-2 | Yield (%) |
|---|---|---|
| 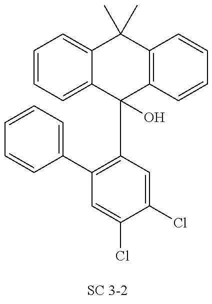 [SMILES: CC1(C)c2ccccc2C(O)(c2cc(Cl)c(Cl)cc2-c2ccccc2)c2ccccc21] | 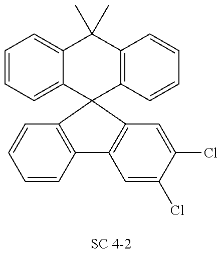 [SMILES: CC1(C)c2ccccc2C2(c3ccccc3-c3cc(Cl)c(Cl)cc32)c2ccccc21] | 76.5 |

### 2. Synthesis of an Intermediate Y 1-X

### Synthesis of an Intermediate A 1-1

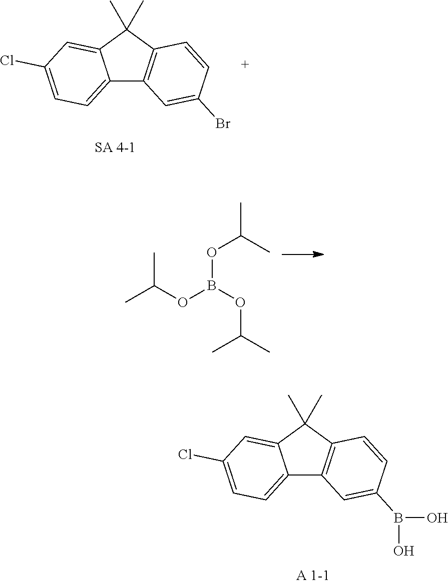

Nitrogen was introduced into a three-necked flask equipped with a mechanical stirrer, a thermometer, and a dropping funnel at 0.100 L/min to allow nitrogen replacement for 15 min, then the intermediate SA 4-1 (111 g, 360.4 mmol) and THF (896 mL) were added, and a resulting mixture was cooled with liquid nitrogen to −80° C. to −90° C. A solution of tert-butyllithium (t-BuLi) in THF (20 mL, 180.2 mmol) was added dropwise, and after the dropwise addition, a resulting mixture was stirred at the above temperature for 1 h; triisopropyl borate (83.64 mL, 360.4 mmol) was added, and a resulting mixture was gradually warmed to room temperature and then stirred for 3 h. A hydrochloric acid aqueous solution (600 mL) was added, and a resulting mixture was stirred at room temperature for 1.5 h; and after the reaction was completed, a precipitate was filtered out, then the precipiate was washed with water and diethyl ether, and then vacuum-dried to obtain the intermediate A 1-1 (88.56 g, yield: 90.1%).

The intermediates Y 1-X (each X was an integer of 1 to 18 and each Y was A, B, or C) shown in table 10 below were each synthesized with reference to the synthesis method of the intermediate A 1-1, wherein the intermediates SY Z-X (each X was an integer of 1 to 7, each Y was A, B, or C, and each Z was 4, 6, 8, or 9) or the reactant SA 4-6 were used instead of the intermediate SA 4-1.

TABLE 10

| Intermediate SY Z-X | Intermediate Y 1-X | Yield (%) |
|---|---|---|
| 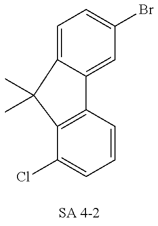 [SMILES: CC1(C)c2ccc(Br)cc2-c2cccc(Cl)c21] | 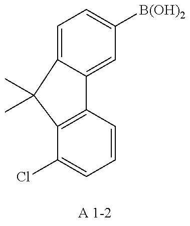 [SMILES: CC1(C)c2ccc(B(O)O)cc2-c2cccc(Cl)c21] | 88.1 |
| 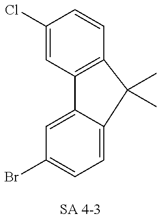 [SMILES: CC1(C)c2ccc(Cl)cc2-c2cc(Br)ccc21] | 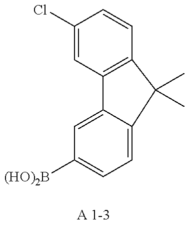 [SMILES: CC1(C)c2ccc(Cl)cc2-c2cc(B(O)O)ccc21] | 89.3 |
| 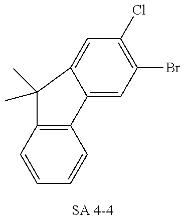 [SMILES: CC1(C)c2ccccc2-c2cc(Br)c(Cl)cc21] | 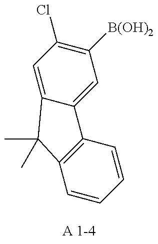 [SMILES: CC1(C)c2ccccc2-c2cc(B(O)O)c(Cl)cc21] | 84.5 |
| 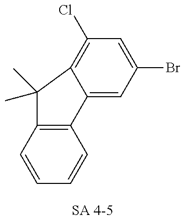 [SMILES: CC1(C)c2ccccc2-c2cc(Br)cc(Cl)c21] | 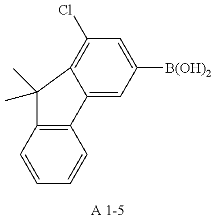 [SMILES: CC1(C)c2ccccc2-c2cc(B(O)O)cc(Cl)c21] | 86.3 |
| 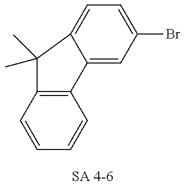 [SMILES: CC1(C)c2ccccc2-c2cc(Br)ccc21] | 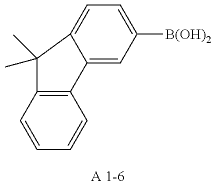 [SMILES: CC1(C)c2ccccc2-c2cc(B(O)O)ccc21] | 91.0 |
| 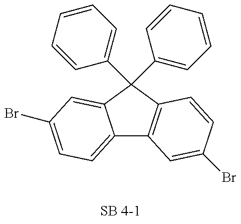 [SMILES: Brc1ccc2c(c1)-c1ccc(Br)cc1C2(c1ccccc1)c1ccccc1] | 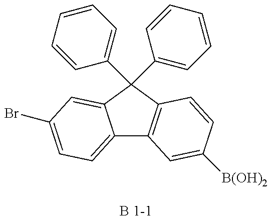 [SMILES: OB(O)c1ccc2c(c1)-c1ccc(Br)cc1C2(c1ccccc1)c1ccccc1] | 56.6 |
| 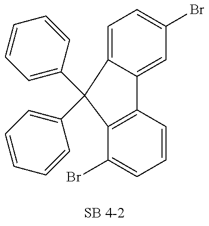 [SMILES: Brc1ccc2c(c1)-c1cccc(Br)c1C2(c1ccccc1)c1ccccc1] | 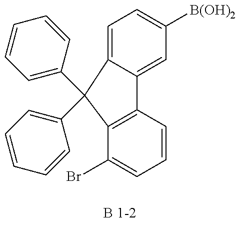 [SMILES: OB(O)c1ccc2c(c1)-c1cccc(Br)c1C2(c1ccccc1)c1ccccc1] | 54.8 |
| 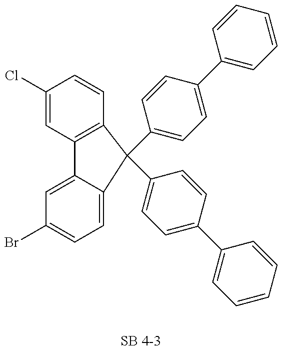 [SMILES: Clc1ccc2c(c1)-c1cc(Br)ccc1C2(c1ccc(-c2ccccc2)cc1)c1ccc(-c2ccccc2)cc1] | 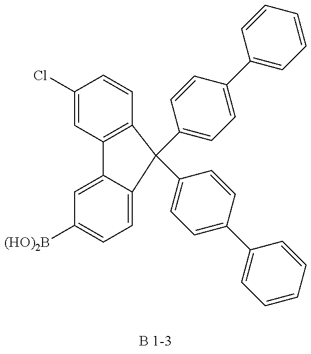 [SMILES: OB(O)c1ccc2c(c1)-c1cc(Cl)ccc1C2(c1ccc(-c2ccccc2)cc1)c1ccc(-c2ccccc2)cc1] | 81.8 |
| 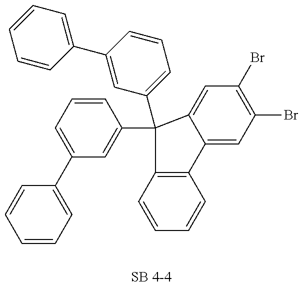 [SMILES: Brc1cc2c(cc1Br)C(c1cccc(-c3ccccc3)c1)(c1cccc(-c3ccccc3)c1)c1ccccc1-2] | 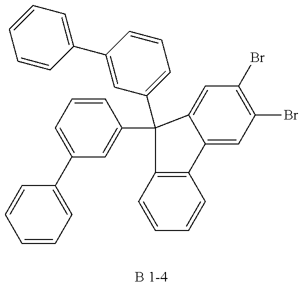 [SMILES: Brc1cc2c(cc1Br)C(c1cccc(-c3ccccc3)c1)(c1cccc(-c3ccccc3)c1)c1ccccc1-2] | 52.3 |
| 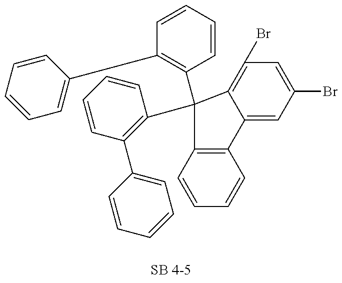 [SMILES: Brc1cc(Br)c2c(c1)-c1ccccc1C2(c1ccccc1-c1ccccc1)c1ccccc1-c1ccccc1] | 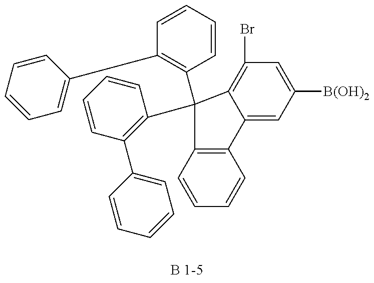 [SMILES: OB(O)c1cc(Br)c2c(c1)-c1ccccc1C2(c1ccccc1-c1ccccc1)c1ccccc1-c1ccccc1] | 58.3 |
| 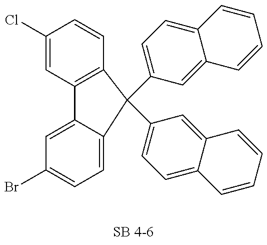 [SMILES: Clc1ccc2c(c1)-c1cc(Br)ccc1C2(c1ccc2ccccc2c1)c1ccc2ccccc2c1] | 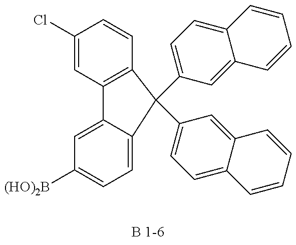 [SMILES: OB(O)c1ccc2c(c1)-c1cc(Cl)ccc1C2(c1ccc2ccccc2c1)c1ccc2ccccc2c1] | 51.2 |
| 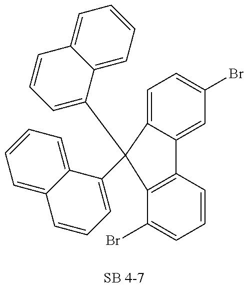 [SMILES: Brc1ccc2c(c1)-c1cccc(Br)c1C2(c1cccc2ccccc12)c1cccc2ccccc12] | 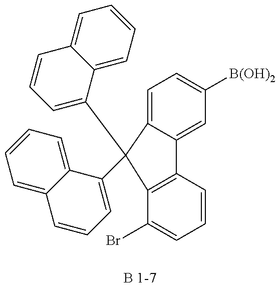 [SMILES: OB(O)c1ccc2c(c1)-c1cccc(Br)c1C2(c1cccc2ccccc12)c1cccc2ccccc12] | 56.4 |
| 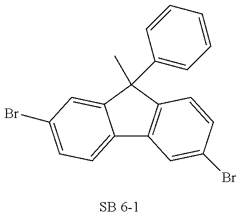 [SMILES: CC1(c2ccccc2)c2ccc(Br)cc2-c2ccc(Br)cc21] | 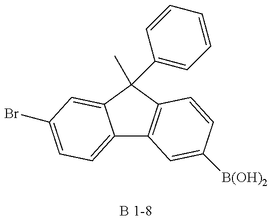 [SMILES: CC1(c2ccccc2)c2ccc(B(O)O)cc2-c2ccc(Br)cc21] | 51.2 |
| 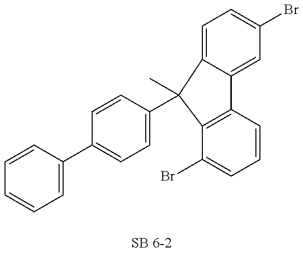 [SMILES: CC1(c2ccc(-c3ccccc3)cc2)c2ccc(Br)cc2-c2cccc(Br)c21] | 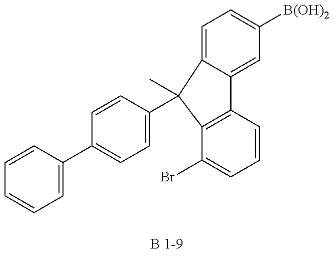 [SMILES: CC1(c2ccc(-c3ccccc3)cc2)c2ccc(B(O)O)cc2-c2cccc(Br)c21] | 54.2 |
| 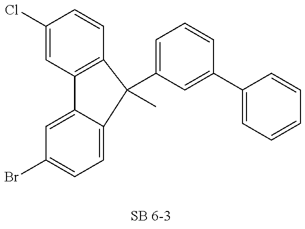 [SMILES: CC1(c2cccc(-c3ccccc3)c2)c2ccc(Cl)cc2-c2cc(Br)ccc21] | 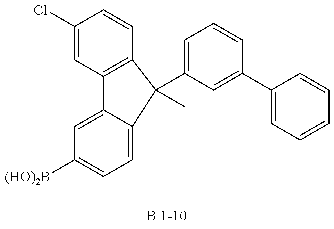 [SMILES: CC1(c2cccc(-c3ccccc3)c2)c2ccc(Cl)cc2-c2cc(B(O)O)ccc21] | 79.8 |
| 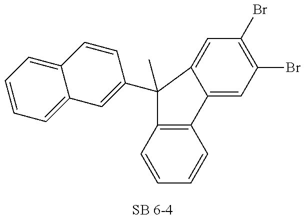 [SMILES: CC1(c2ccc3ccccc3c2)c2ccccc2-c2cc(Br)c(Br)cc21] | 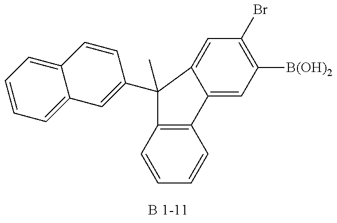 [SMILES: CC1(c2ccc3ccccc3c2)c2ccccc2-c2cc(B(O)O)c(Br)cc21] | 52.3 |
| 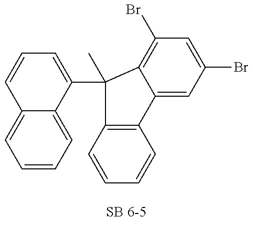 [SMILES: CC1(c2cccc3ccccc23)c2ccccc2-c2cc(Br)cc(Br)c21] | 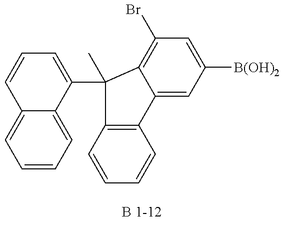 [SMILES: CC1(c2cccc3ccccc23)c2ccccc2-c2cc(B(O)O)cc(Br)c21] | 51.4 |
| 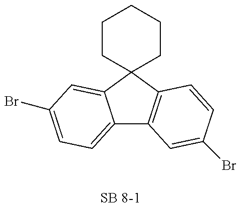 [SMILES: Brc1ccc2c(c1)-c1ccc(Br)cc1C21CCCCC1] | 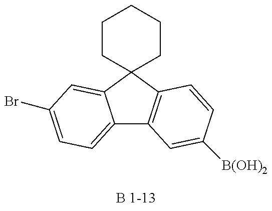 [SMILES: OB(O)c1ccc2c(c1)-c1ccc(Br)cc1C21CCCCC1] | 51.6 |
| 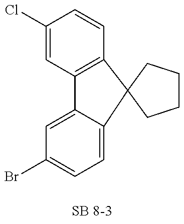 [SMILES: Clc1ccc2c(c1)-c1cc(Br)ccc1C21CCCC1] | 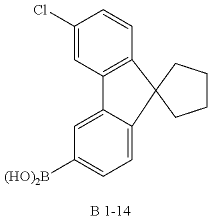 [SMILES: OB(O)c1ccc2c(c1)-c1cc(Cl)ccc1C21CCCC1] | 80.9 |
| 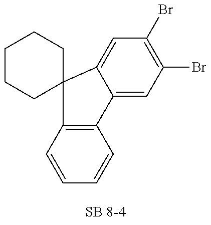 [SMILES: Brc1cc2c(cc1Br)C1(CCCCC1)c1ccccc1-2] | 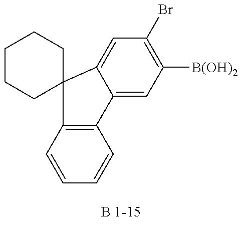 [SMILES: OB(O)c1cc2c(cc1Br)C1(CCCCC1)c1ccccc1-2] | 51.3 |
| 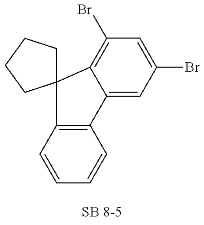 [SMILES: Brc1cc(Br)c2c(c1)-c1ccccc1C21CCCC1] | 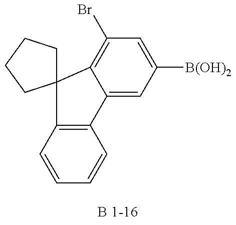 [SMILES: OB(O)c1cc(Br)c2c(c1)-c1ccccc1C21CCCC1] | 52.1 |
| 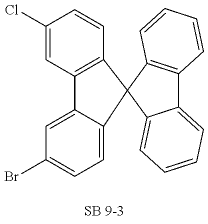 [SMILES: Clc1ccc2c(c1)-c1cc(Br)ccc1C21c2ccccc2-c2ccccc21] | 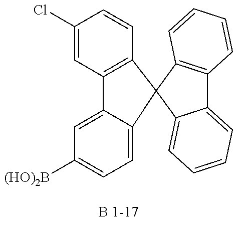 [SMILES: OB(O)c1ccc2c(c1)-c1cc(Cl)ccc1C21c2ccccc2-c2ccccc21] | 81.1 |
| 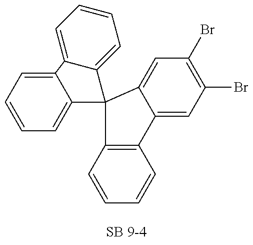 [SMILES: Brc1cc2c(cc1Br)C1(c3ccccc3-c3ccccc31)c1ccccc1-2] | 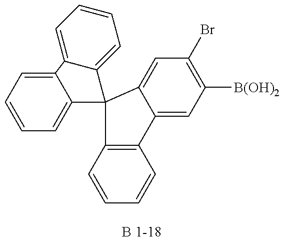 [SMILES: OB(O)c1cc2c(cc1Br)C1(c3ccccc3-c3ccccc31)c1ccccc1-2] | 53.1 |
| 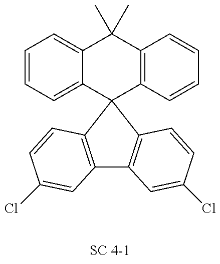 [SMILES: CC1(C)c2ccccc2C2(c3ccc(Cl)cc3-c3cc(Cl)ccc32)c2ccccc21] | 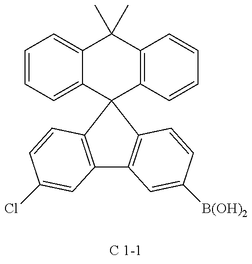 [SMILES: CC1(C)c2ccccc2C2(c3ccc(Cl)cc3-c3cc(B(O)O)ccc32)c2ccccc21] | 50.2 |
| 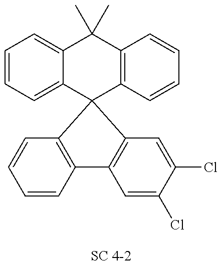 [SMILES: CC1(C)c2ccccc2C2(c3ccccc3-c3cc(Cl)c(Cl)cc32)c2ccccc21] | 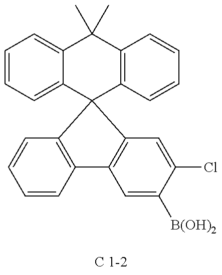 [SMILES: CC1(C)c2ccccc2C2(c3ccccc3-c3cc(B(O)O)c(Cl)cc32)c2ccccc21] | 51.0 |

### 3. Synthesis of an Intermediate Y 3-X

### Synthesis of an Intermediate A 3-1

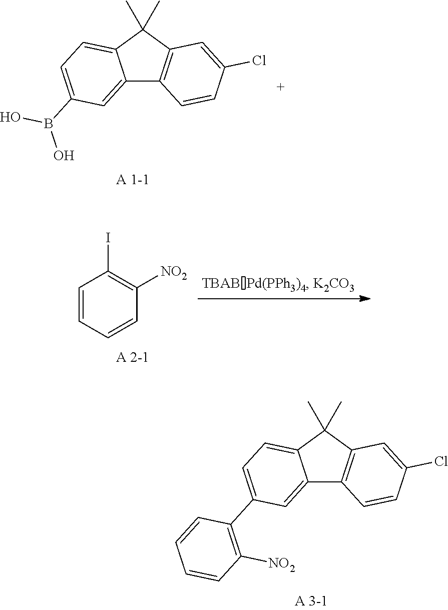

Nitrogen was introduced into a three-necked flask equipped with a mechanical stirrer, a thermometer, and a spherical condenser at 0.100 L/min to allow nitrogen replacement for 15 min, then the intermediate A 1-1 (87.4 g, 320.14 mmol), a reactant A 2-1 (79.72 g, 320.14 mmol), THF (528 mL), and H₂O (176 mL) were added, and a resulting mixture was heated to reflux and stirred until a resulting solution was clear. TBAB (2.06 g, 6.40 mmol), tetrakis(triphenylphosphine)palladium (3.70 g, 3.20 mmol), and potassium carbonate (66.28 g, 480.22 mmol) were added, and a reaction was kept to reflux and stirred for 15 h. After the reaction was completed, a resulting reaction system was cooled to room temperature; DCM was added for extraction, and the separated organic phase was washed with water until neutral. The organic phase was dried with anhydrous magnesium sulfate, and filtered, and a filtrate was concentrated in vacuum to obtain a crude product; and the crude product was purified by silica gel column chromatography to obtain the intermediate SA 3-1 (76.2 g, yield: 68.1%).

The intermediates Y 3-X (each X was an integer of 1 to 18 and each Y was A, B, or C) shown in table 11 below were each synthesized with reference to the synthesis method of the intermediate A 3-1, wherein the intermediates Y 1-X (each X was an integer of 1 to 18 and each Y was A, B, or C) were used instead of the intermediate A 1-1 and the reactants A 2-X (each X was an integer of 1 to 6) were used instead of the reactant A 2-1.

TABLE 11

| | | | Yield |
|---|---|---|---|
| Intermediate Y 1-X | Reactant A 2-X | Intermediate Y 3-X | (%) |
| 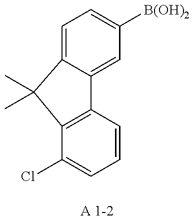 [SMILES: CC1(C)c2ccc(B(O)O)cc2-c2cccc(Cl)c21] | 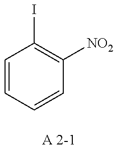 | 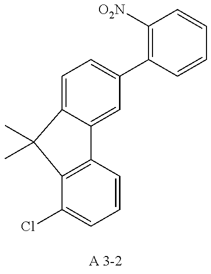 [SMILES: CC1(C)c2ccc(-c3ccccc3[N+](=O)[O-])cc2-c2cccc(Cl)c21] | 67.5 |
| 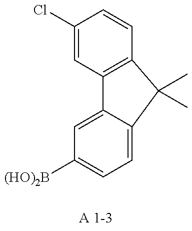 [SMILES: CC1(C)c2ccc(Cl)cc2-c2cc(B(O)O)ccc21] | | 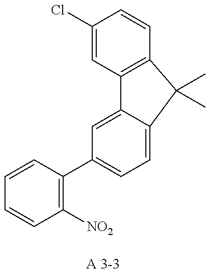 [SMILES: CC1(C)c2ccc(Cl)cc2-c2cc(-c3ccccc3[N+](=O)[O-])ccc21] | 68.1 |
| 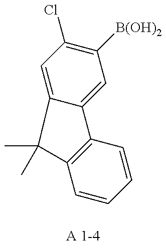 [SMILES: CC1(C)c2ccccc2-c2cc(B(O)O)c(Cl)cc21] | | 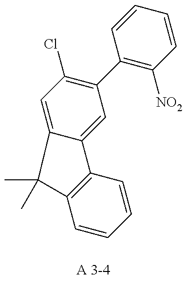 [SMILES: CC1(C)c2ccccc2-c2cc(-c3ccccc3[N+](=O)[O-])c(Cl)cc21] | 69.1 |
| 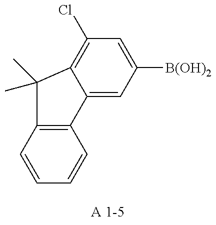 [SMILES: CC1(C)c2ccccc2-c2cc(B(O)O)cc(Cl)c21] | | 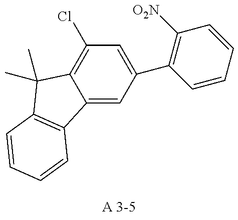 [SMILES: CC1(C)c2ccccc2-c2cc(-c3ccccc3[N+](=O)[O-])cc(Cl)c21] | 66.3 |
| 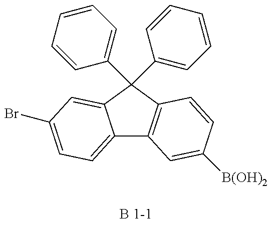 [SMILES: OB(O)c1ccc2c(c1)-c1ccc(Br)cc1C2(c1ccccc1)c1ccccc1] | 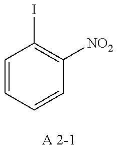 | 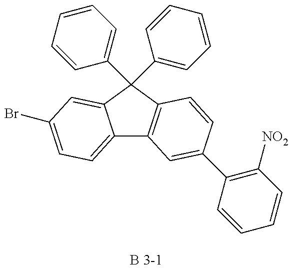 [SMILES: O=[N+]([O-])c1ccccc1-c1ccc2c(c1)-c1ccc(Br)cc1C2(c1ccccc1)c1ccccc1] | 67.1 |
| 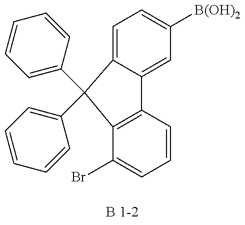 [SMILES: OB(O)c1ccc2c(c1)-c1cccc(Br)c1C2(c1ccccc1)c1ccccc1] | | 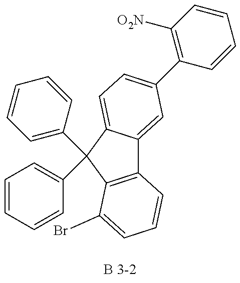 [SMILES: O=[N+]([O-])c1ccccc1-c1ccc2c(c1)-c1cccc(Br)c1C2(c1ccccc1)c1ccccc1] | 65.3 |
| 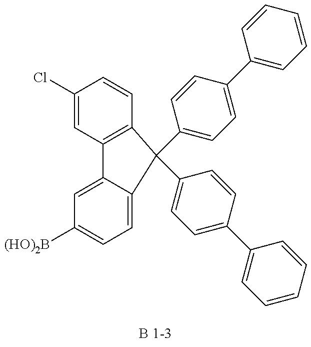 [SMILES: OB(O)c1ccc2c(c1)-c1cc(Cl)ccc1C2(c1ccc(-c2ccccc2)cc1)c1ccc(-c2ccccc2)cc1] | | 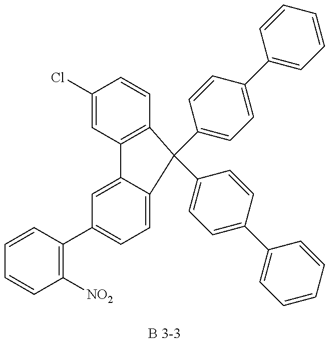 [SMILES: O=[N+]([O-])c1ccccc1-c1ccc2c(c1)-c1cc(Cl)ccc1C2(c1ccc(-c2ccccc2)cc1)c1ccc(-c2ccccc2)cc1] | 63.9 |
| 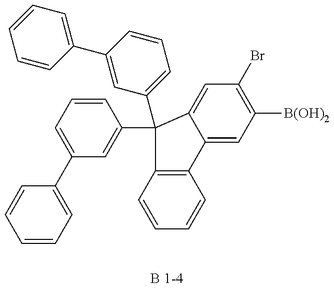 [SMILES: OB(O)c1cc2c(cc1Br)C(c1cccc(-c3ccccc3)c1)(c1cccc(-c3ccccc3)c1)c1ccccc1-2] | | 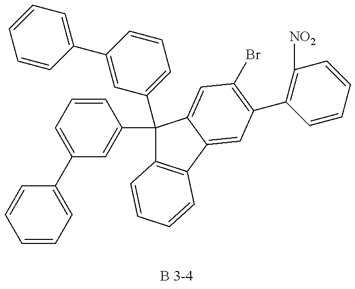 [SMILES: O=[N+]([O-])c1ccccc1-c1cc2c(cc1Br)C(c1cccc(-c3ccccc3)c1)(c1cccc(-c3ccccc3)c1)c1ccccc1-2] | 61.7 |
| 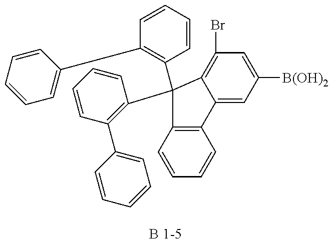 [SMILES: OB(O)c1cc(Br)c2c(c1)-c1ccccc1C2(c1ccccc1-c1ccccc1)c1ccccc1-c1ccccc1] | | 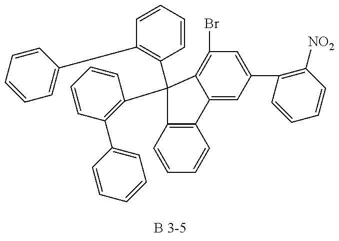 [SMILES: O=[N+]([O-])c1ccccc1-c1cc(Br)c2c(c1)-c1ccccc1C2(c1ccccc1-c1ccccc1)c1ccccc1-c1ccccc1] | 66.2 |
| 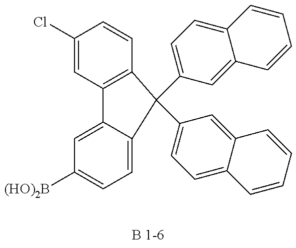 [SMILES: OB(O)c1ccc2c(c1)-c1cc(Cl)ccc1C2(c1ccc2ccccc2c1)c1ccc2ccccc2c1] | | 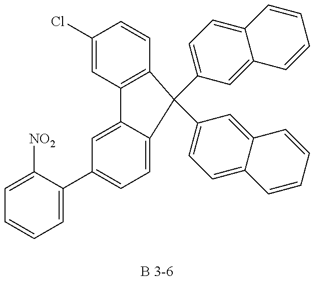 [SMILES: O=[N+]([O-])c1ccccc1-c1ccc2c(c1)-c1cc(Cl)ccc1C2(c1ccc2ccccc2c1)c1ccc2ccccc2c1] | 65.1 |
| 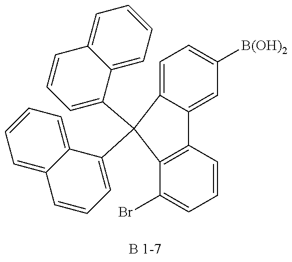 [SMILES: OB(O)c1ccc2c(c1)-c1cccc(Br)c1C2(c1cccc2ccccc12)c1cccc2ccccc12] | | 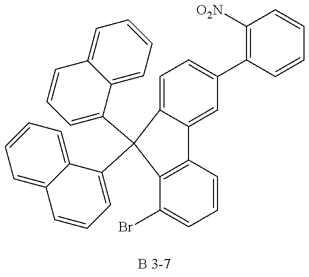 [SMILES: O=[N+]([O-])c1ccccc1-c1ccc2c(c1)-c1cccc(Br)c1C2(c1cccc2ccccc12)c1cccc2ccccc12] | 64.9 |
| 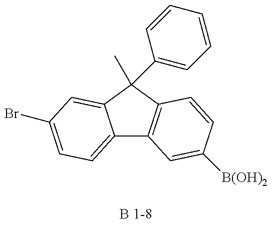 [SMILES: CC1(c2ccccc2)c2ccc(B(O)O)cc2-c2ccc(Br)cc21] | | 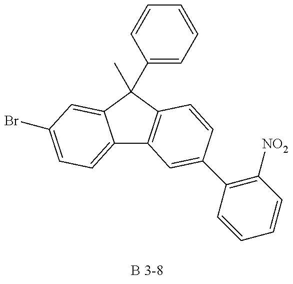 [SMILES: CC1(c2ccccc2)c2ccc(-c3ccccc3[N+](=O)[O-])cc2-c2ccc(Br)cc21] | 65.3 |
| 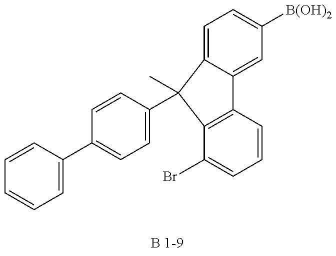 [SMILES: CC1(c2ccc(-c3ccccc3)cc2)c2ccc(B(O)O)cc2-c2cccc(Br)c21] | | 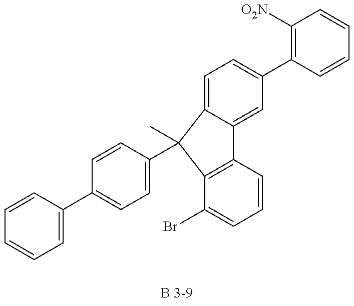 [SMILES: CC1(c2ccc(-c3ccccc3)cc2)c2ccc(-c3ccccc3[N+](=O)[O-])cc2-c2cccc(Br)c21] | 66.1 |
| 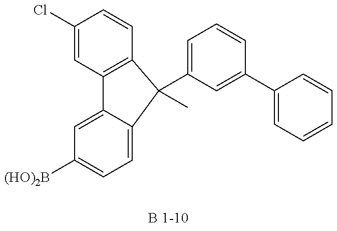 [SMILES: CC1(c2cccc(-c3ccccc3)c2)c2ccc(Cl)cc2-c2cc(B(O)O)ccc21] | | 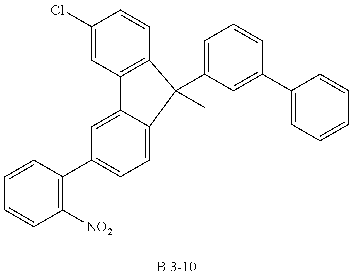 [SMILES: CC1(c2cccc(-c3ccccc3)c2)c2ccc(Cl)cc2-c2cc(-c3ccccc3[N+](=O)[O-])ccc21] | 65.8 |
| 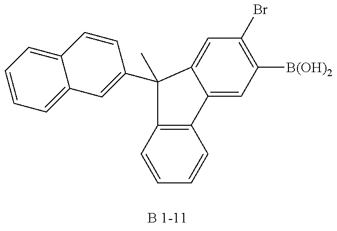 [SMILES: CC1(c2ccc3ccccc3c2)c2ccccc2-c2cc(B(O)O)c(Br)cc21] | | 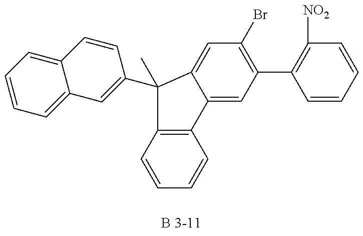 [SMILES: CC1(c2ccc3ccccc3c2)c2ccccc2-c2cc(-c3ccccc3[N+](=O)[O-])c(Br)cc21] | 63.8 |
| 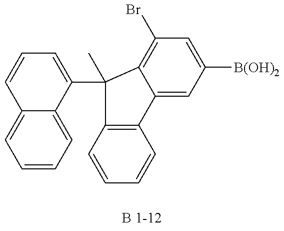 [SMILES: CC1(c2cccc3ccccc23)c2ccccc2-c2cc(B(O)O)cc(Br)c21] | | 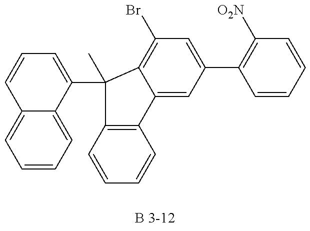 [SMILES: CC1(c2cccc3ccccc23)c2ccccc2-c2cc(-c3ccccc3[N+](=O)[O-])cc(Br)c21] | 65.7 |
| 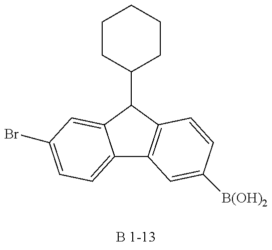 [SMILES: OB(O)c1ccc2c(c1)-c1ccc(Br)cc1C2C1CCCCC1] | | 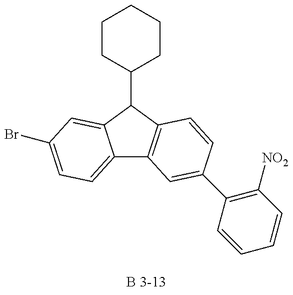 [SMILES: O=[N+]([O-])c1ccccc1-c1ccc2c(c1)-c1ccc(Br)cc1C2C1CCCCC1] | 66.8 |
| 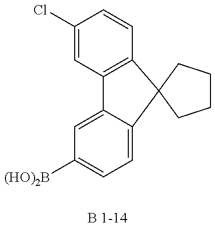 [SMILES: OB(O)c1ccc2c(c1)-c1cc(Cl)ccc1C21CCCC1] | | 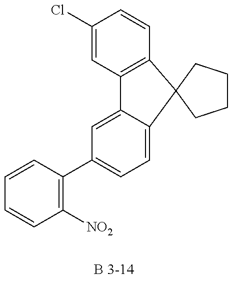 [SMILES: O=[N+]([O-])c1ccccc1-c1ccc2c(c1)-c1cc(Cl)ccc1C21CCCC1] | 66.8 |
| 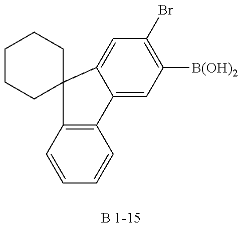 [SMILES: OB(O)c1cc2c(cc1Br)C1(CCCCC1)c1ccccc1-2] | | 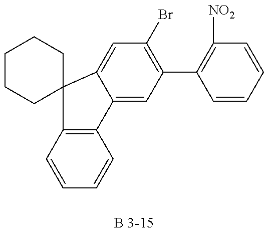 [SMILES: O=[N+]([O-])c1ccccc1-c1cc2c(cc1Br)C1(CCCCC1)c1ccccc1-2] | 63.1 |
| 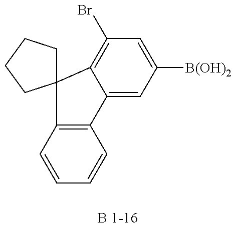 [SMILES: OB(O)c1cc(Br)c2c(c1)-c1ccccc1C21CCCC1] | | 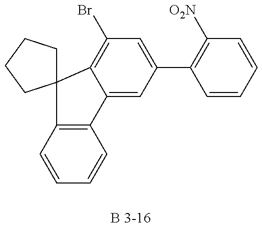 [SMILES: O=[N+]([O-])c1ccccc1-c1cc(Br)c2c(c1)-c1ccccc1C21CCCC1] | 66.7 |
| 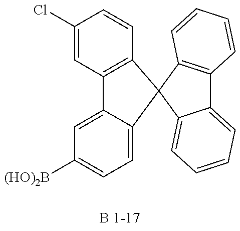 [SMILES: OB(O)c1ccc2c(c1)-c1cc(Cl)ccc1C21c2ccccc2-c2ccccc21] | | 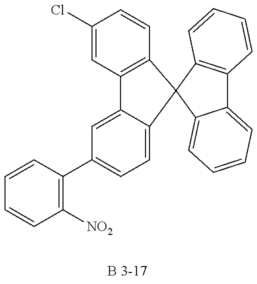 [SMILES: O=[N+]([O-])c1ccccc1-c1ccc2c(c1)-c1cc(Cl)ccc1C21c2ccccc2-c2ccccc21] | 66.5 |
| 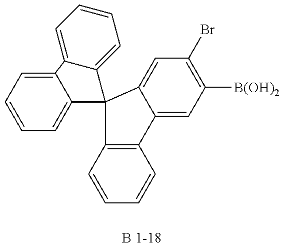 [SMILES: OB(O)c1cc2c(cc1Br)C1(c3ccccc3-c3ccccc31)c1ccccc1-2] | | 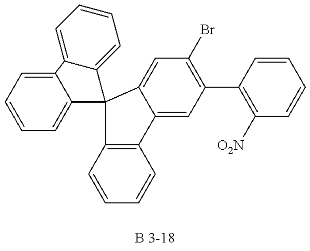 [SMILES: O=[N+]([O-])c1ccccc1-c1cc2c(cc1Br)C1(c3ccccc3-c3ccccc31)c1ccccc1-2] | 64.3 |
| 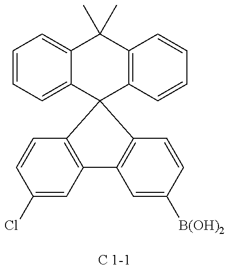 [SMILES: CC1(C)c2ccccc2C2(c3ccc(Cl)cc3-c3cc(B(O)O)ccc32)c2ccccc21] | | 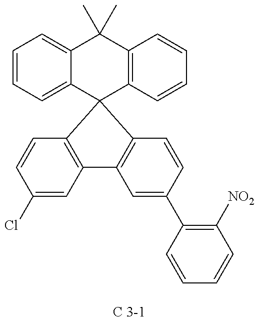 [SMILES: CC1(C)c2ccccc2C2(c3ccc(Cl)cc3-c3cc(-c4ccccc4[N+](=O)[O-])ccc32)c2ccccc21] | 67.3 |
| 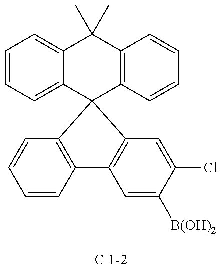 [SMILES: CC1(C)c2ccccc2C2(c3ccccc3-c3cc(B(O)O)c(Cl)cc32)c2ccccc21] | | 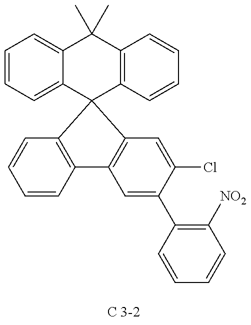 [SMILES: CC1(C)c2ccccc2C2(c3ccccc3-c3cc(-c4ccccc4[N+](=O)[O-])c(Cl)cc32)c2ccccc21] | 66.8 |
| 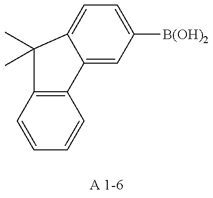 [SMILES: CC1(C)c2ccccc2-c2cc(B(O)O)ccc21] | 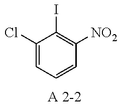 [SMILES: O=[N+]([O-])c1cccc(Cl)c1I] | 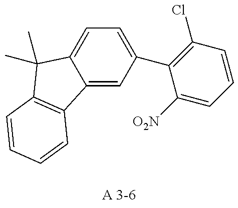 [SMILES: CC1(C)c2ccccc2-c2cc(-c3c(Cl)cccc3[N+](=O)[O-])ccc21] | 67.1 |
| | 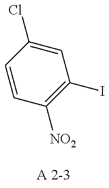 [SMILES: O=[N+]([O-])c1ccc(Cl)cc1I] | 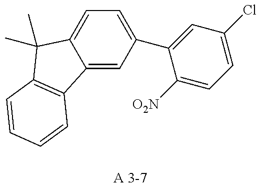 [SMILES: CC1(C)c2ccccc2-c2cc(-c3cc(Cl)ccc3[N+](=O)[O-])ccc21] | 68.3 |
| | 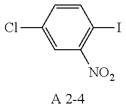 [SMILES: O=[N+]([O-])c1cc(Cl)ccc1I] | 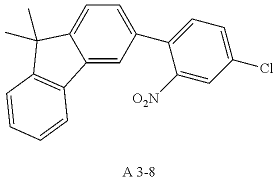 [SMILES: CC1(C)c2ccccc2-c2cc(-c3ccc(Cl)cc3[N+](=O)[O-])ccc21] | 66.6 |
| | 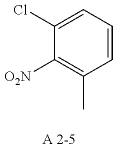 | 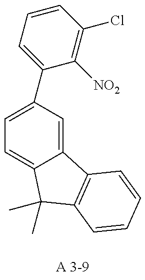 [SMILES: CC1(C)c2ccccc2-c2cc(-c3cccc(Cl)c3[N+](=O)[O-])ccc21] | 68.3 |
| | 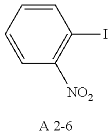 | 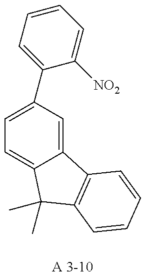 [SMILES: CC1(C)c2ccccc2-c2cc(-c3ccccc3[N+](=O)[O-])ccc21] | 69.1 |

### 4. Synthesis of an Intermediate Y 4-X

### Synthesis of an Intermediate A4-1

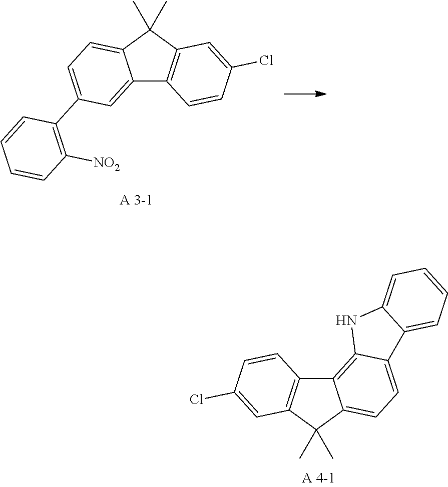 [SMILES: CC1(C)c2ccc(-c3ccccc3[N+](=O)[O-])cc2-c2ccc(Cl)cc21 ; CC1(C)c2cc(Cl)ccc2-c2c1ccc1c2[nH]c2ccccc21]

Nitrogen was introduced into a three-necked flask equipped with a mechanical stirrer, a thermometer, and a spherical condenser at 0.100 L/min to allow nitrogen replacement for 15 min, then the intermediate A 3-1 (74 g, 211.43 mmol), triphenylphosphine (11.1 g, 42.32 mmol), and o-dichlorobenzene (100 mL) were added, and a resulting mixture was heated to 170° C. to 190° C. and stirred for 16 h. After the reaction was completed, a resulting reaction mixture was cooled to room temperature and washed with water, a resulting organic phase was separated, dried with anhydrous magnesium sulfate, and filtered, and a filtrate was concentrated in vacuum to obtain a crude product; and the crude product was purified by silica gel column chromatography to obtain the intermediate A 4-1 (43.70 g, yield: 65.0%).

The intermediates Y 4-X (each X was an integer of 1 to 18 and each Y was A, B, or C) shown in table 12 below were each synthesized with reference to the synthesis method of the intermediate A 4-1, wherein the intermediates Y 3-X (each X was an integer of 1 to 18 and each Y was A, B, or C) were used instead of the intermediate A 3-1.

TABLE 12

| Intermediate Y 3-X | Intermediate Y 4-X | Yield (%) |
|---|---|---|
| 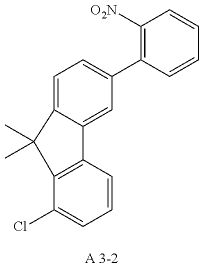 [SMILES: CC1(C)c2ccc(-c3ccccc3[N+](=O)[O-])cc2-c2cccc(Cl)c21] | 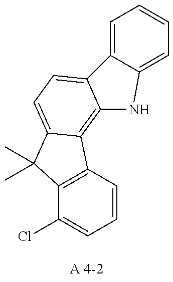 [SMILES: CC1(C)c2ccc3c([nH]c4ccccc43)c2-c2cccc(Cl)c21] | 66.2 |
| 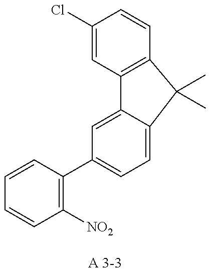 [SMILES: CC1(C)c2ccc(Cl)cc2-c2cc(-c3ccccc3[N+](=O)[O-])ccc21] | 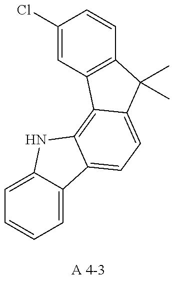 [SMILES: CC1(C)c2ccc(Cl)cc2-c2c1ccc1c2[nH]c2ccccc21] | 63.6 |
| 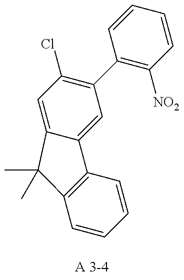 [SMILES: CC1(C)c2ccccc2-c2cc(-c3ccccc3[N+](=O)[O-])c(Cl)cc21] | 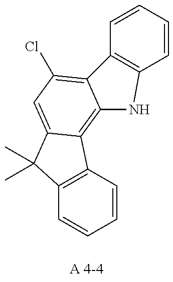 [SMILES: CC1(C)c2ccccc2-c2c1cc(Cl)c1c2[nH]c2ccccc21] | 61.1 |
| 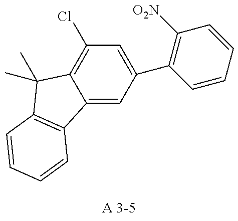 [SMILES: CC1(C)c2ccccc2-c2cc(-c3ccccc3[N+](=O)[O-])cc(Cl)c21] | 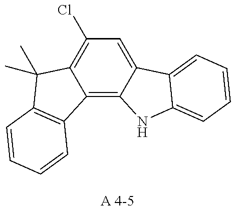 [SMILES: CC1(C)c2ccccc2-c2c1c(Cl)cc1c2[nH]c2ccccc21] | 66.3 |
| 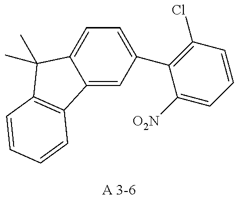 [SMILES: CC1(C)c2ccccc2-c2cc(-c3c(Cl)cccc3[N+](=O)[O-])ccc21] | 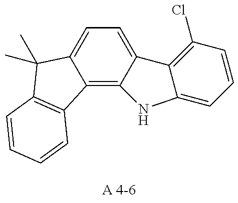 [SMILES: CC1(C)c2ccccc2-c2c1ccc1c2[nH]c2cccc(Cl)c21] | 63.8 |
| 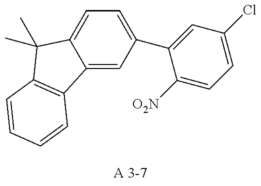 [SMILES: CC1(C)c2ccccc2-c2cc(-c3cc(Cl)ccc3[N+](=O)[O-])ccc21] | 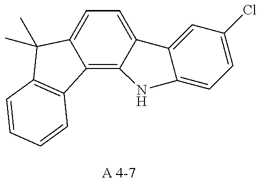 [SMILES: CC1(C)c2ccccc2-c2c1ccc1c2[nH]c2ccc(Cl)cc21] | 65.3 |
| 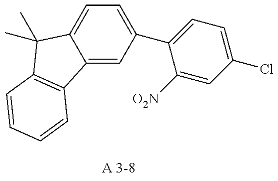 [SMILES: CC1(C)c2ccccc2-c2cc(-c3ccc(Cl)cc3[N+](=O)[O-])ccc21] | 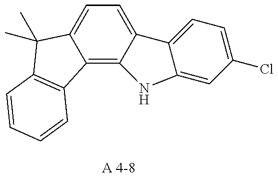 [SMILES: CC1(C)c2ccccc2-c2c1ccc1c2[nH]c2cc(Cl)ccc21] | 66.1 |
| 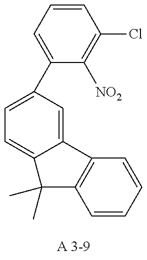 [SMILES: CC1(C)c2ccccc2-c2cc(-c3cccc(Cl)c3[N+](=O)[O-])ccc21] | 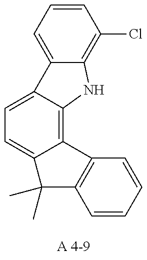 [SMILES: CC1(C)c2ccccc2-c2c1ccc1c2[nH]c2c(Cl)cccc21] | 62.9 |
| 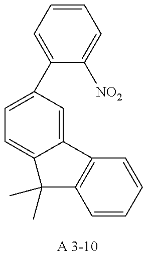 [SMILES: CC1(C)c2ccccc2-c2cc(-c3ccccc3[N+](=O)[O-])ccc21] | 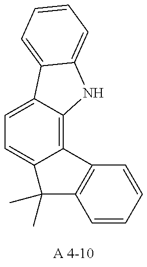 [SMILES: CC1(C)c2ccccc2-c2c1ccc1c2[nH]c2ccccc21] | 67.4 |
| 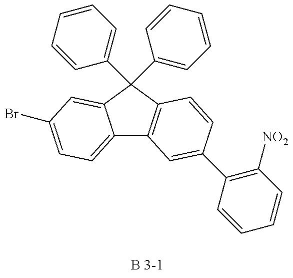 [SMILES: O=[N+]([O-])c1ccccc1-c1ccc2c(c1)-c1ccc(Br)cc1C2(c1ccccc1)c1ccccc1] | 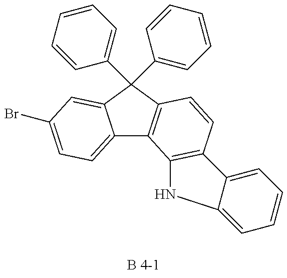 [SMILES: Brc1ccc2c(c1)C(c1ccccc1)(c1ccccc1)c1ccc3c([nH]c4ccccc43)c1-2] | 63.2 |
| 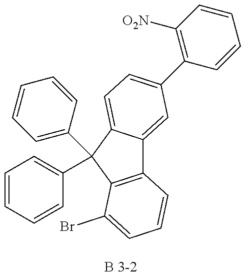 [SMILES: O=[N+]([O-])c1ccccc1-c1ccc2c(c1)-c1cccc(Br)c1C2(c1ccccc1)c1ccccc1] | 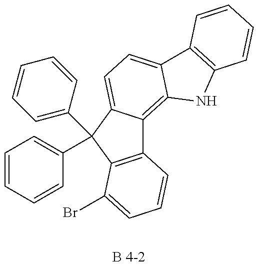 [SMILES: Brc1cccc2c1C(c1ccccc1)(c1ccccc1)c1ccc3c([nH]c4ccccc43)c1-2] | 65.1 |
| 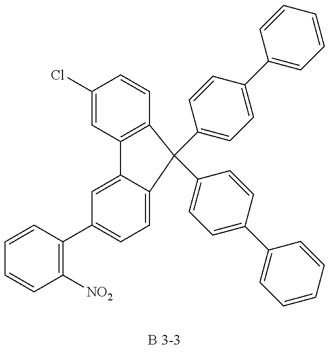 [SMILES: O=[N+]([O-])c1ccccc1-c1ccc2c(c1)-c1cc(Cl)ccc1C2(c1ccc(-c2ccccc2)cc1)c1ccc(-c2ccccc2)cc1] | 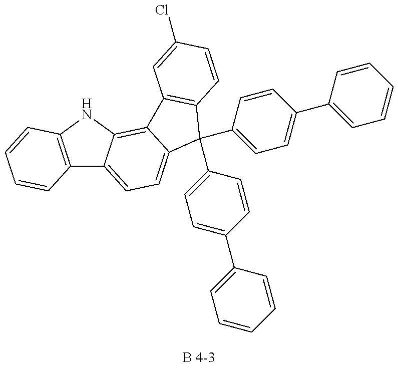 [SMILES: Clc1ccc2c(c1)-c1c(ccc3c1[nH]c1ccccc13)C2(c1ccc(-c2ccccc2)cc1)c1ccc(-c2ccccc2)cc1] | 63.2 |
| 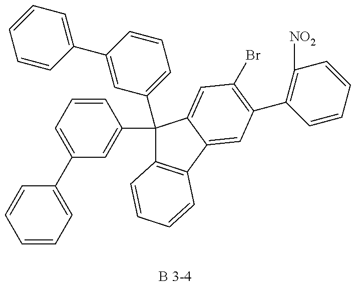 [SMILES: O=[N+]([O-])c1ccccc1-c1cc2c(cc1Br)C(c1cccc(-c3ccccc3)c1)(c1cccc(-c3ccccc3)c1)c1ccccc1-2] | 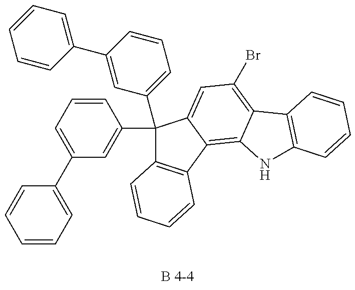 [SMILES: Brc1cc2c(c3[nH]c4ccccc4c13)-c1ccccc1C2(c1cccc(-c2ccccc2)c1)c1cccc(-c2ccccc2)c1] | 66.1 |
| 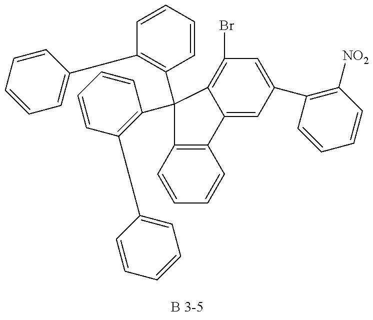 [SMILES: O=[N+]([O-])c1ccccc1-c1cc(Br)c2c(c1)-c1ccccc1C2(c1ccccc1-c1ccccc1)c1ccccc1-c1ccccc1] | 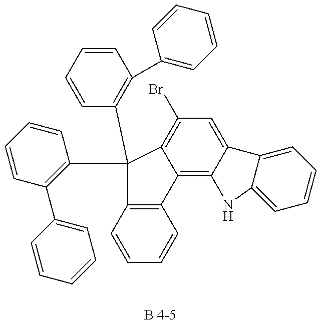 [SMILES: Brc1cc2c([nH]c3ccccc32)c2c1C(c1ccccc1-c1ccccc1)(c1ccccc1-c1ccccc1)c1ccccc1-2] | 63.1 |
| 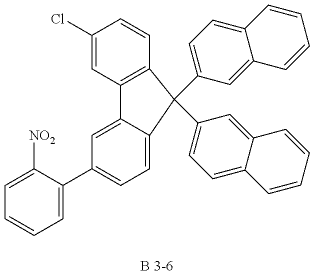 [SMILES: O=[N+]([O-])c1ccccc1-c1ccc2c(c1)-c1cc(Cl)ccc1C2(c1ccc2ccccc2c1)c1ccc2ccccc2c1] | 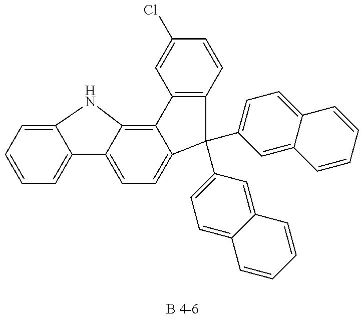 [SMILES: Clc1ccc2c(c1)-c1c(ccc3c1[nH]c1ccccc13)C2(c1ccc2ccccc2c1)c1ccc2ccccc2c1] | 64.8 |
| 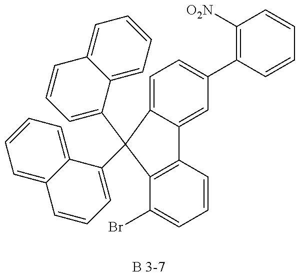 [SMILES: O=[N+]([O-])c1ccccc1-c1ccc2c(c1)-c1cccc(Br)c1C2(c1cccc2ccccc12)c1cccc2ccccc12] | 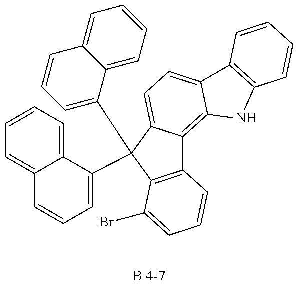 [SMILES: Brc1cccc2c1C(c1cccc3ccccc13)(c1cccc3ccccc13)c1ccc3c([nH]c4ccccc43)c1-2] | 63.8 |
| 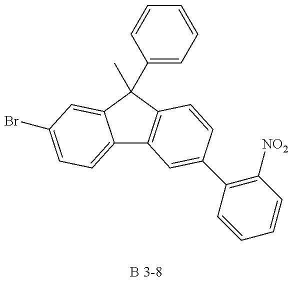 [SMILES: CC1(c2ccccc2)c2ccc(-c3ccccc3[N+](=O)[O-])cc2-c2ccc(Br)cc21] | 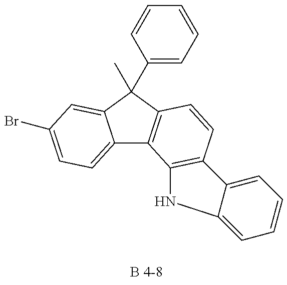 [SMILES: CC1(c2ccccc2)c2cc(Br)ccc2-c2c1ccc1c2[nH]c2ccccc21] | 66.1 |
| 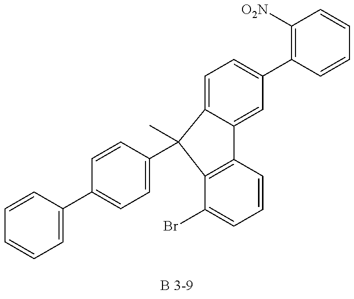 [SMILES: CC1(c2ccc(-c3ccccc3)cc2)c2ccc(-c3ccccc3[N+](=O)[O-])cc2-c2cccc(Br)c21] | 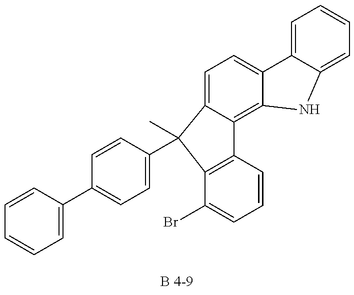 [SMILES: CC1(c2ccc(-c3ccccc3)cc2)c2ccc3c([nH]c4ccccc43)c2-c2cccc(Br)c21] | 65.4 |
| 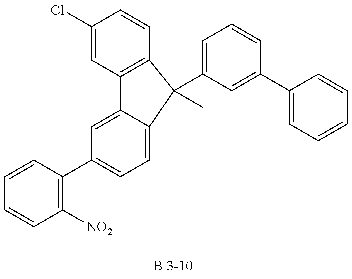 [SMILES: CC1(c2cccc(-c3ccccc3)c2)c2ccc(Cl)cc2-c2cc(-c3ccccc3[N+](=O)[O-])ccc21] | 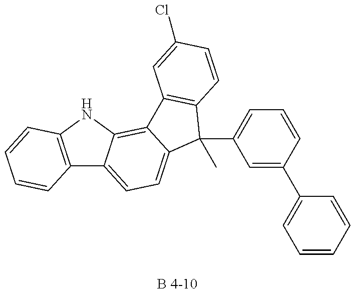 [SMILES: CC1(c2cccc(-c3ccccc3)c2)c2ccc(Cl)cc2-c2c1ccc1c2[nH]c2ccccc21] | 66.2 |
| 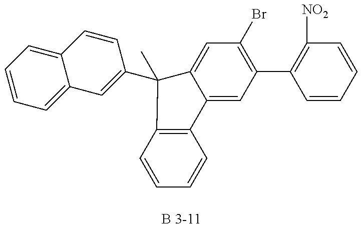 [SMILES: CC1(c2ccc3ccccc3c2)c2ccccc2-c2cc(-c3ccccc3[N+](=O)[O-])c(Br)cc21] | 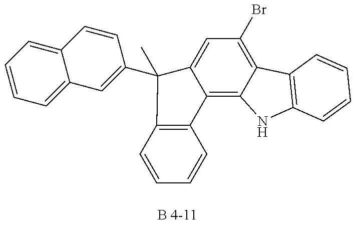 [SMILES: CC1(c2ccc3ccccc3c2)c2ccccc2-c2c1cc(Br)c1c2[nH]c2ccccc21] | 65.3 |
| 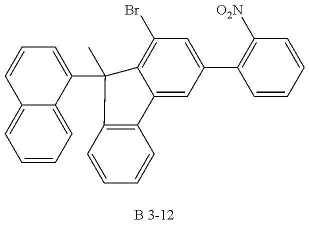 [SMILES: CC1(c2cccc3ccccc23)c2ccccc2-c2cc(-c3ccccc3[N+](=O)[O-])cc(Br)c21] | 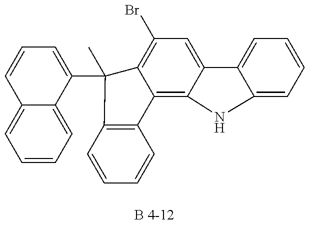 [SMILES: CC1(c2cccc3ccccc23)c2ccccc2-c2c1c(Br)cc1c2[nH]c2ccccc21] | 64.8 |
| 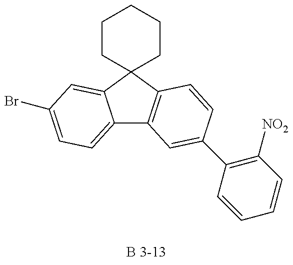 [SMILES: O=[N+]([O-])c1ccccc1-c1ccc2c(c1)-c1ccc(Br)cc1C21CCCCC1] | 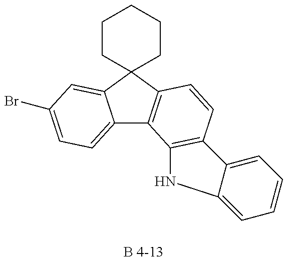 [SMILES: Brc1ccc2c(c1)C1(CCCCC1)c1ccc3c([nH]c4ccccc43)c1-2] | 66.4 |
| 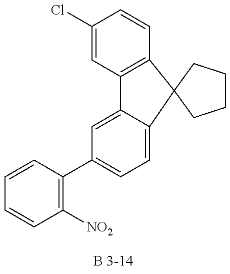 [SMILES: O=[N+]([O-])c1ccccc1-c1ccc2c(c1)-c1cc(Cl)ccc1C21CCCC1] | 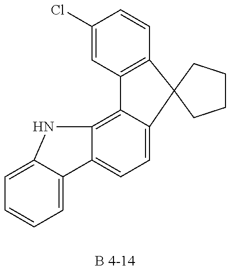 [SMILES: Clc1ccc2c(c1)-c1c(ccc3c1[nH]c1ccccc13)C21CCCC1] | 64.9 |
| 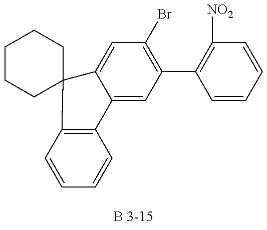 [SMILES: O=[N+]([O-])c1ccccc1-c1cc2c(cc1Br)C1(CCCCC1)c1ccccc1-2] | 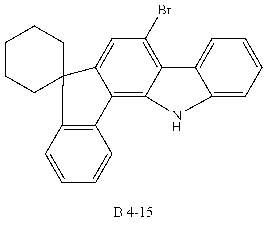 [SMILES: Brc1cc2c(c3[nH]c4ccccc4c13)-c1ccccc1C21CCCCC1] | 63.1 |
| 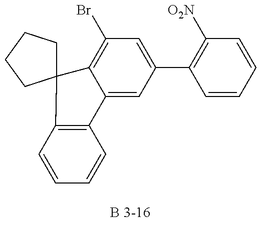 [SMILES: O=[N+]([O-])c1ccccc1-c1cc(Br)c2c(c1)-c1ccccc1C21CCCC1] | 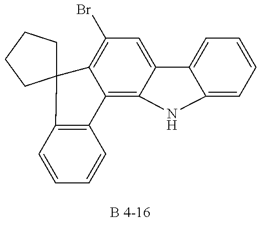 [SMILES: Brc1cc2c([nH]c3ccccc32)c2c1C1(CCCC1)c1ccccc1-2] | 64.1 |
| 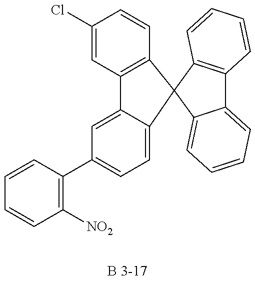 [SMILES: O=[N+]([O-])c1ccccc1-c1ccc2c(c1)-c1cc(Cl)ccc1C21c2ccccc2-c2ccccc21] | 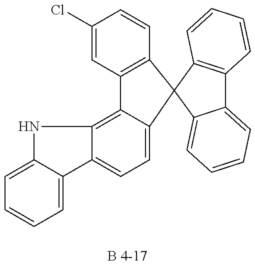 [SMILES: Clc1ccc2c(c1)-c1c(ccc3c1[nH]c1ccccc13)C21c2ccccc2-c2ccccc21] | 35.8 |
| 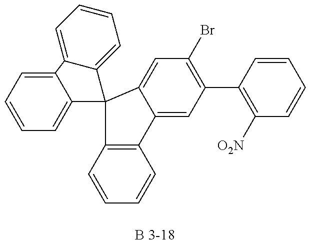 [SMILES: O=[N+]([O-])c1ccccc1-c1cc2c(cc1Br)C1(c3ccccc3-c3ccccc31)c1ccccc1-2] | 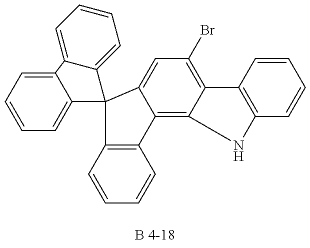 [SMILES: Brc1cc2c(c3[nH]c4ccccc4c13)-c1ccccc1C21c2ccccc2-c2ccccc21] | 66.7 |
| 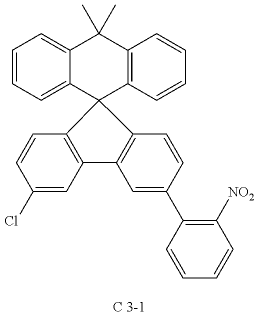 [SMILES: CC1(C)c2ccccc2C2(c3ccc(Cl)cc3-c3cc(-c4ccccc4[N+](=O)[O-])ccc32)c2ccccc21] | 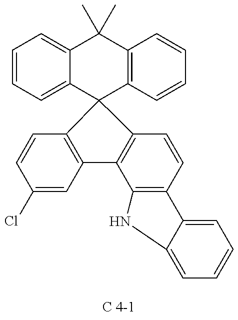 [SMILES: CC1(C)c2ccccc2C2(c3ccc(Cl)cc3-c3c2ccc2c3[nH]c3ccccc32)c2ccccc21] | 66.2 |
| 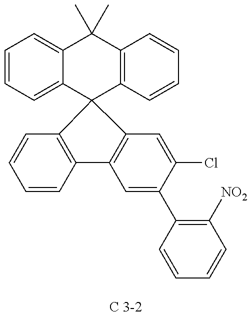 [SMILES: CC1(C)c2ccccc2C2(c3ccccc3-c3cc(-c4ccccc4[N+](=O)[O-])c(Cl)cc32)c2ccccc21] | 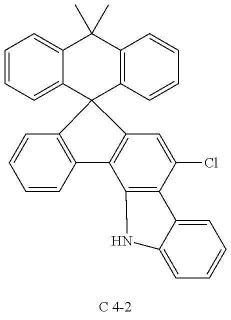 [SMILES: CC1(C)c2ccccc2C2(c3ccccc3-c3c2cc(Cl)c2c3[nH]c3ccccc32)c2ccccc21] | 65.7 |

### 5. Synthesis of an Intermediate Y 6-X

### Synthesis of an Intermediate A 6-1

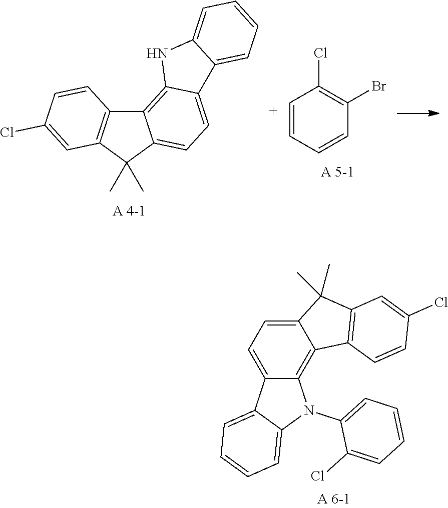 [SMILES: CC1(C)c2cc(Cl)ccc2-c2c1ccc1c2[nH]c2ccccc21 ; Clc1ccccc1Br ; CC1(C)c2cc(Cl)ccc2-c2c1ccc1c3ccccc3n(-c3ccccc3Cl)c21]

Nitrogen was introduced into a three-necked flask equipped with a mechanical stirrer, a thermometer, and a spherical condenser at 0.100 L/min to allow nitrogen replacement for 15 min, then the intermediate A 4-1 (37.75 g, 118.71 mmol), a reactant A 5-1 (22.67 g, 118.71 mmol), cesium carbonate (3.87 g, 11.87 mmol), and DMSO (320 mL) were added, and a resulting mixture was stirred for 15 h; after the reaction was completed, a resulting reaction system was cooled to room temperature; toluene was added for extraction, a separated organic phase was dried with anhydrous magnesium sulfate, and filtered, and a filtrate was concentrated in vacuum to obtain a crude product; and the crude product was purified by silica gel column chromatography to obtain the intermediate A 6-1 (34.04 g, yield: 67.3%).

The intermediates Y 6-X (each X was an integer of 1 to 18 and each Y was A, B, or C) shown in table 13 below were each synthesized with reference to the synthesis method of the intermediate A 6-1, wherein the intermediates Y 4-X (each X was an integer of 1 to 18 and each Y was A, B, or C) were used instead of the intermediate A 4-1 and the reactants A 5-X (each X was an integer of 1 to 4) were used instead of the reactant A 5-1.

TABLE 13

| | | | Yield |
|---|---|---|---|
| Intermediate Y 4-X | Reactant A 5-X | Intermediate Y 6-X | (%) |
| 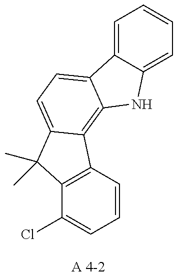 [SMILES: CC1(C)c2ccc3c([nH]c4ccccc43)c2-c2cccc(Cl)c21] | 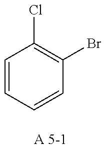 | 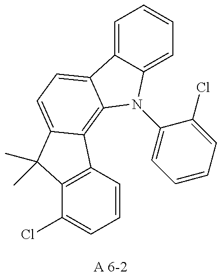 [SMILES: CC1(C)c2ccc3c4ccccc4n(-c4ccccc4Cl)c3c2-c2cccc(Cl)c21] | 65.1 |
| 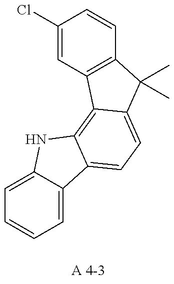 [SMILES: CC1(C)c2ccc(Cl)cc2-c2c1ccc1c2[nH]c2ccccc21] | | 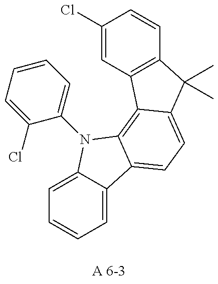 [SMILES: CC1(C)c2ccc(Cl)cc2-c2c1ccc1c3ccccc3n(-c3ccccc3Cl)c21] | 66.4 |
| 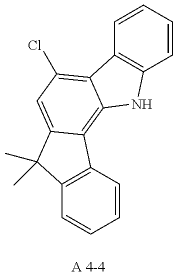 [SMILES: CC1(C)c2ccccc2-c2c1cc(Cl)c1c2[nH]c2ccccc21] | | 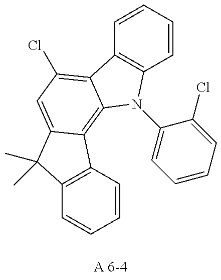 [SMILES: CC1(C)c2ccccc2-c2c1cc(Cl)c1c3ccccc3n(-c3ccccc3Cl)c21] | 67.7 |
| 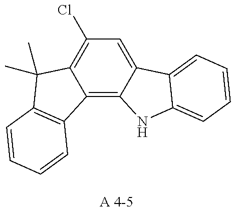 [SMILES: CC1(C)c2ccccc2-c2c1c(Cl)cc1c2[nH]c2ccccc21] | | 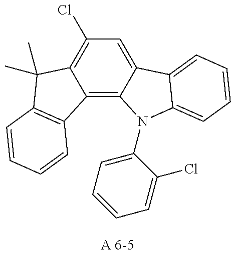 [SMILES: CC1(C)c2ccccc2-c2c1c(Cl)cc1c3ccccc3n(-c3ccccc3Cl)c21] | 65.3 |
| 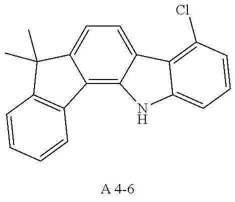 [SMILES: CC1(C)c2ccccc2-c2c1ccc1c2[nH]c2cccc(Cl)c21] | | 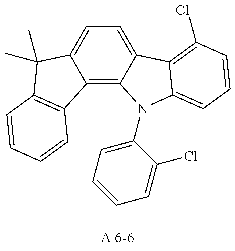 [SMILES: CC1(C)c2ccccc2-c2c1ccc1c3c(Cl)cccc3n(-c3ccccc3Cl)c21] | 67.4 |
| 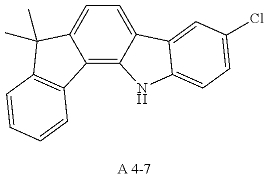 [SMILES: CC1(C)c2ccccc2-c2c1ccc1c2[nH]c2ccc(Cl)cc21] | | 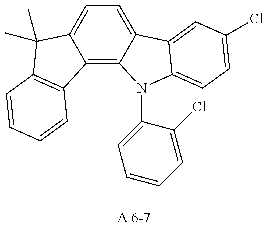 [SMILES: CC1(C)c2ccccc2-c2c1ccc1c3cc(Cl)ccc3n(-c3ccccc3Cl)c21] | 65.8 |
| 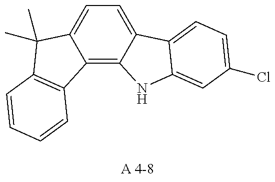 [SMILES: CC1(C)c2ccccc2-c2c1ccc1c2[nH]c2cc(Cl)ccc21] | | 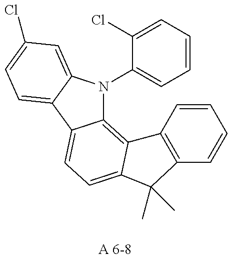 [SMILES: CC1(C)c2ccccc2-c2c1ccc1c3ccc(Cl)cc3n(-c3ccccc3Cl)c21] | 65.1 |
| 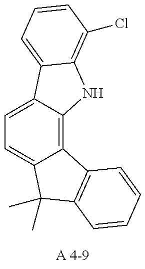 [SMILES: CC1(C)c2ccccc2-c2c1ccc1c2[nH]c2c(Cl)cccc21] | | 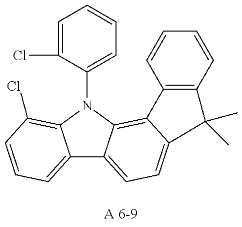 [SMILES: CC1(C)c2ccccc2-c2c1ccc1c3cccc(Cl)c3n(-c3ccccc3Cl)c21] | 63.5 |
| 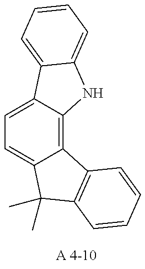 [SMILES: CC1(C)c2ccccc2-c2c1ccc1c2[nH]c2ccccc21] | 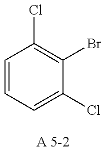 | 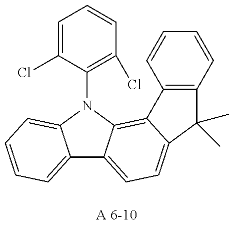 [SMILES: CC1(C)c2ccccc2-c2c1ccc1c3ccccc3n(-c3c(Cl)cccc3Cl)c21] | 62.5 |
| | 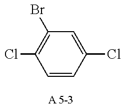 | 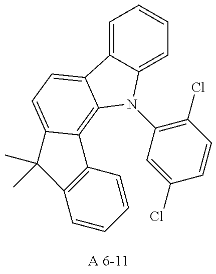 [SMILES: CC1(C)c2ccccc2-c2c1ccc1c3ccccc3n(-c3cc(Cl)ccc3Cl)c21] | 63.5 |
| | 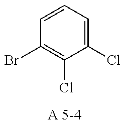 | 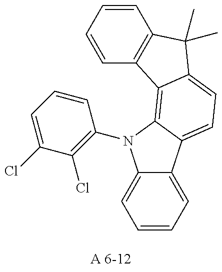 [SMILES: CC1(C)c2ccccc2-c2c1ccc1c3ccccc3n(-c3cccc(Cl)c3Cl)c21] | 66.4 |
| 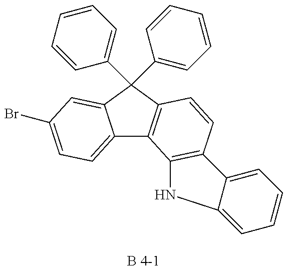 [SMILES: Brc1ccc2c(c1)C(c1ccccc1)(c1ccccc1)c1ccc3c([nH]c4ccccc43)c1-2] | 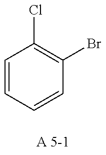 | 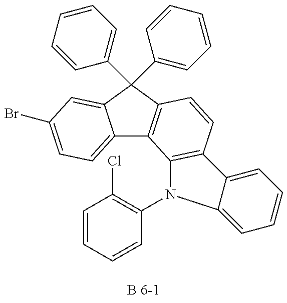 [SMILES: Clc1ccccc1-n1c2ccccc2c2ccc3c(c21)-c1ccc(Br)cc1C3(c1ccccc1)c1ccccc1] | 67.5 |
| 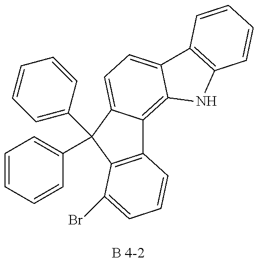 [SMILES: Brc1cccc2c1C(c1ccccc1)(c1ccccc1)c1ccc3c([nH]c4ccccc43)c1-2] | 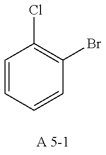 | 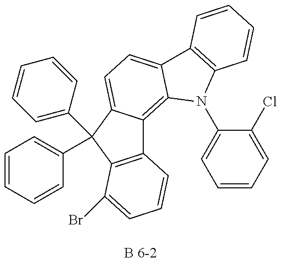 [SMILES: Clc1ccccc1-n1c2ccccc2c2ccc3c(c21)-c1cccc(Br)c1C3(c1ccccc1)c1ccccc1] | 65.3 |
| 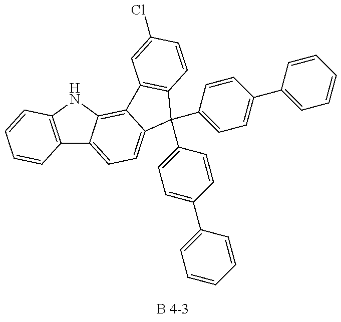 [SMILES: Clc1ccc2c(c1)-c1c(ccc3c1[nH]c1ccccc13)C2(c1ccc(-c2ccccc2)cc1)c1ccc(-c2ccccc2)cc1] | 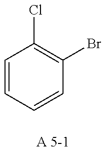 | 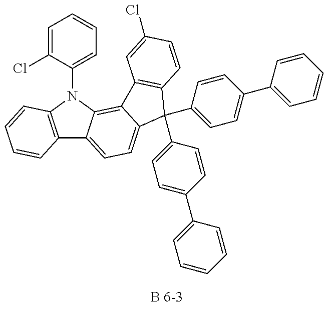 [SMILES: Clc1ccc2c(c1)-c1c(ccc3c4ccccc4n(-c4ccccc4Cl)c13)C2(c1ccc(-c2ccccc2)cc1)c1ccc(-c2ccccc2)cc1] | 66.1 |
| 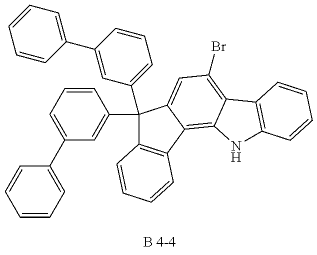 [SMILES: Brc1cc2c(c3[nH]c4ccccc4c13)-c1ccccc1C2(c1cccc(-c2ccccc2)c1)c1cccc(-c2ccccc2)c1] | 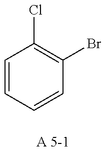 | 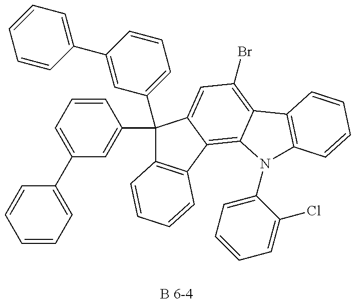 [SMILES: Clc1ccccc1-n1c2ccccc2c2c(Br)cc3c(c21)-c1ccccc1C3(c1cccc(-c2ccccc2)c1)c1cccc(-c2ccccc2)c1] | 66.4 |
| 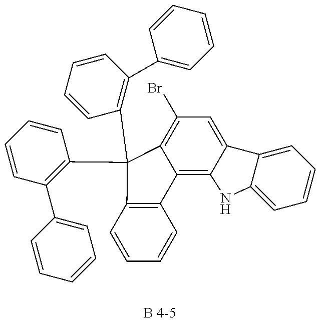 [SMILES: Brc1cc2c([nH]c3ccccc32)c2c1C(c1ccccc1-c1ccccc1)(c1ccccc1-c1ccccc1)c1ccccc1-2] | 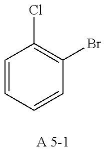 | 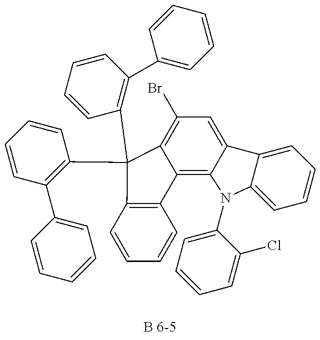 [SMILES: Clc1ccccc1-n1c2ccccc2c2cc(Br)c3c(c21)-c1ccccc1C3(c1ccccc1-c1ccccc1)c1ccccc1-c1ccccc1] | 64.5 |
| 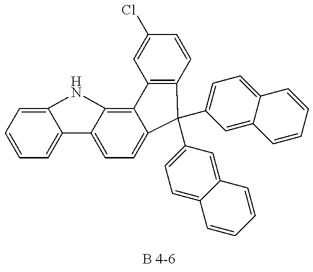 [SMILES: Clc1ccc2c(c1)-c1c(ccc3c1[nH]c1ccccc13)C2(c1ccc2ccccc2c1)c1ccc2ccccc2c1] | 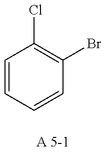 | 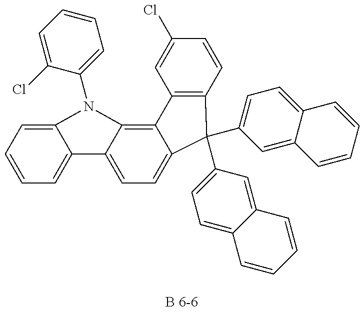 [SMILES: Clc1ccc2c(c1)-c1c(ccc3c4ccccc4n(-c4ccccc4Cl)c13)C2(c1ccc2ccccc2c1)c1ccc2ccccc2c1] | 65.6 |
| 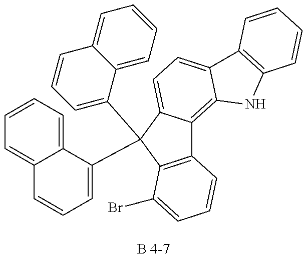 [SMILES: Brc1cccc2c1C(c1cccc3ccccc13)(c1cccc3ccccc13)c1ccc3c([nH]c4ccccc43)c1-2] | 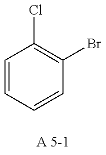 | 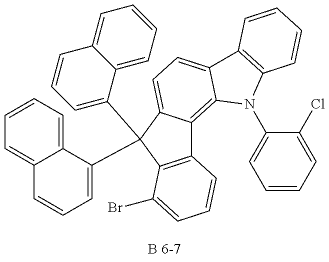 [SMILES: Clc1ccccc1-n1c2ccccc2c2ccc3c(c21)-c1cccc(Br)c1C3(c1cccc2ccccc12)c1cccc2ccccc12] | 66.7 |
| 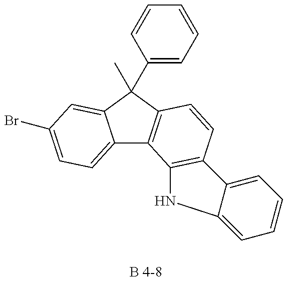 [SMILES: CC1(c2ccccc2)c2cc(Br)ccc2-c2c1ccc1c2[nH]c2ccccc21] | 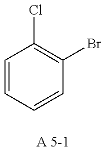 | 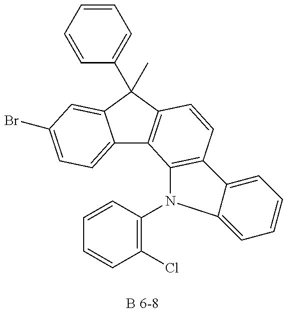 [SMILES: CC1(c2ccccc2)c2cc(Br)ccc2-c2c1ccc1c3ccccc3n(-c3ccccc3Cl)c21] | 67.5 |
| 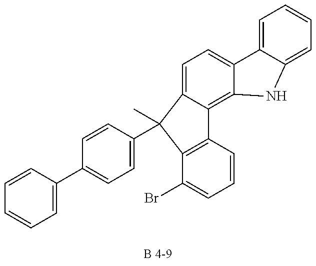 [SMILES: CC1(c2ccc(-c3ccccc3)cc2)c2ccc3c([nH]c4ccccc43)c2-c2cccc(Br)c21] | 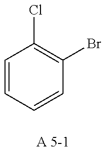 | 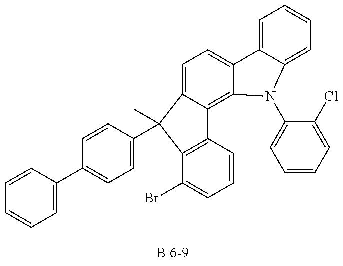 [SMILES: CC1(c2ccc(-c3ccccc3)cc2)c2ccc3c4ccccc4n(-c4ccccc4Cl)c3c2-c2cccc(Br)c21] | 65.2 |
| 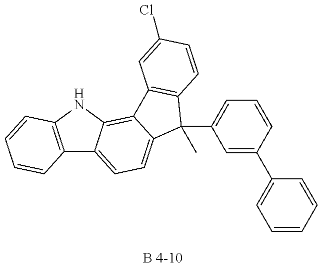 [SMILES: CC1(c2cccc(-c3ccccc3)c2)c2ccc(Cl)cc2-c2c1ccc1c2[nH]c2ccccc21] | 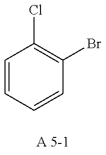 | 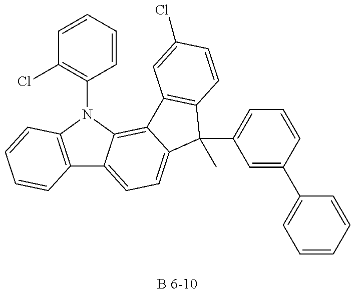 [SMILES: CC1(c2cccc(-c3ccccc3)c2)c2ccc(Cl)cc2-c2c1ccc1c3ccccc3n(-c3ccccc3Cl)c21] | 67.3 |
| 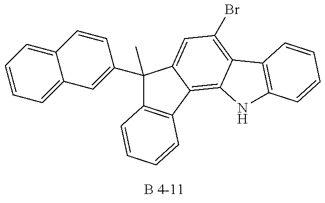 [SMILES: CC1(c2ccc3ccccc3c2)c2ccccc2-c2c1cc(Br)c1c2[nH]c2ccccc21] | 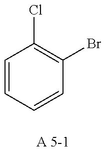 | 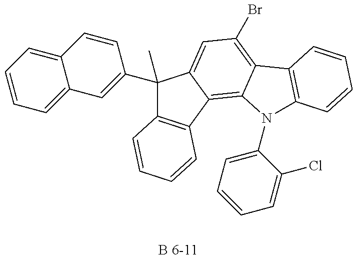 [SMILES: CC1(c2ccc3ccccc3c2)c2ccccc2-c2c1cc(Br)c1c3ccccc3n(-c3ccccc3Cl)c21] | 65.7 |
| 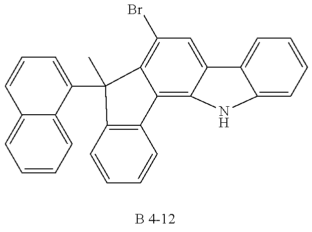 [SMILES: CC1(c2cccc3ccccc23)c2ccccc2-c2c1c(Br)cc1c2[nH]c2ccccc21] | 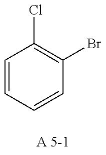 | 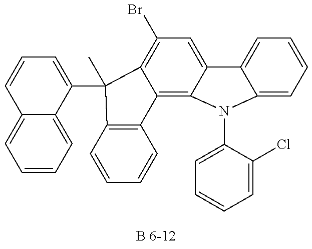 [SMILES: CC1(c2cccc3ccccc23)c2ccccc2-c2c1c(Br)cc1c3ccccc3n(-c3ccccc3Cl)c21] | 66.1 |
| 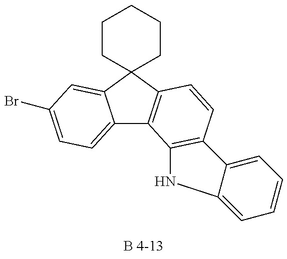 [SMILES: Brc1ccc2c(c1)C1(CCCCC1)c1ccc3c([nH]c4ccccc43)c1-2] | 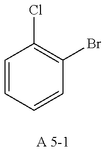 | 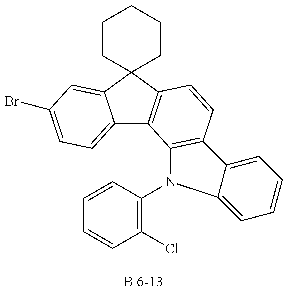 [SMILES: Clc1ccccc1-n1c2ccccc2c2ccc3c(c21)-c1ccc(Br)cc1C31CCCCC1] | 67.6 |
| 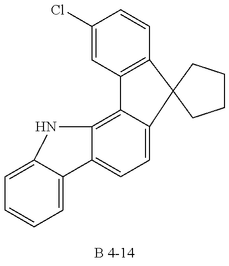 [SMILES: Clc1ccc2c(c1)-c1c(ccc3c1[nH]c1ccccc13)C21CCCC1] | 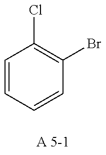 | 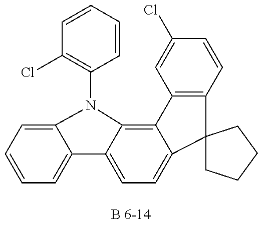 [SMILES: Clc1ccc2c(c1)-c1c(ccc3c4ccccc4n(-c4ccccc4Cl)c13)C21CCCC1] | 66.5 |
| 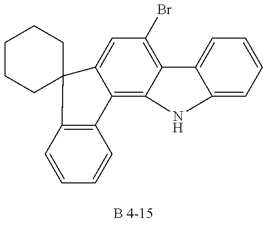 [SMILES: Brc1cc2c(c3[nH]c4ccccc4c13)-c1ccccc1C21CCCCC1] | 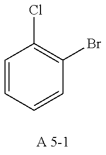 | 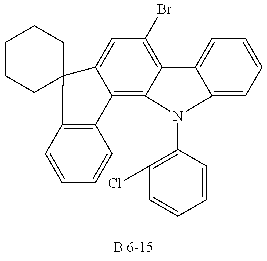 [SMILES: Clc1ccccc1-n1c2ccccc2c2c(Br)cc3c(c21)-c1ccccc1C31CCCCC1] | 65.1 |
| 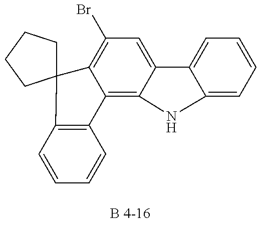 [SMILES: Brc1cc2c([nH]c3ccccc32)c2c1C1(CCCC1)c1ccccc1-2] | 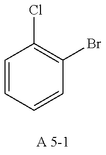 | 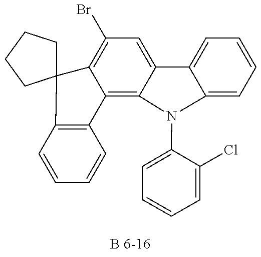 [SMILES: Clc1ccccc1-n1c2ccccc2c2cc(Br)c3c(c21)-c1ccccc1C31CCCC1] | 64.5 |
| 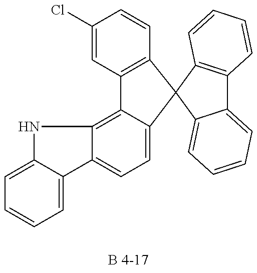 [SMILES: Clc1ccc2c(c1)-c1c(ccc3c1[nH]c1ccccc13)C21c2ccccc2-c2ccccc21] | 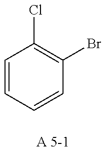 | 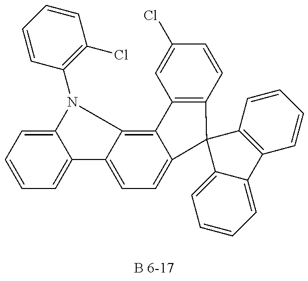 [SMILES: Clc1ccc2c(c1)-c1c(ccc3c4ccccc4n(-c4ccccc4Cl)c13)C21c2ccccc2-c2ccccc21] | 66.8 |
| 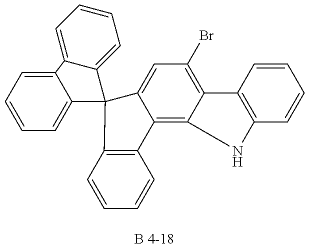 [SMILES: Brc1cc2c(c3[nH]c4ccccc4c13)-c1ccccc1C21c2ccccc2-c2ccccc21] | 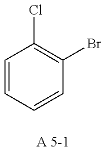 | 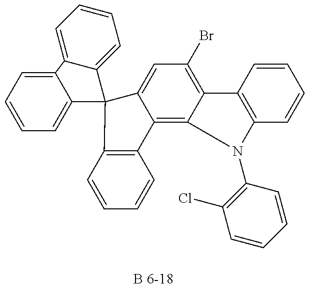 [SMILES: Clc1ccccc1-n1c2ccccc2c2c(Br)cc3c(c21)-c1ccccc1C31c2ccccc2-c2ccccc21] | 67.2 |
| 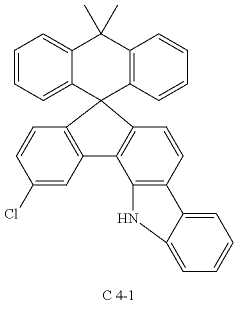 [SMILES: CC1(C)c2ccccc2C2(c3ccc(Cl)cc3-c3c2ccc2c3[nH]c3ccccc32)c2ccccc21] | 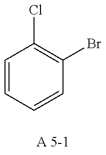 | 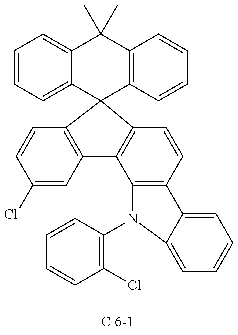 [SMILES: CC1(C)c2ccccc2C2(c3ccc(Cl)cc3-c3c2ccc2c4ccccc4n(-c4ccccc4Cl)c32)c2ccccc21] | 65.1 |
| 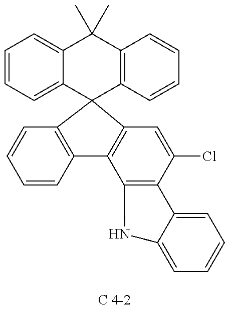 [SMILES: CC1(C)c2ccccc2C2(c3ccccc3-c3c2cc(Cl)c2c3[nH]c3ccccc32)c2ccccc21] | 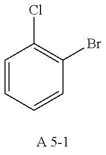 | 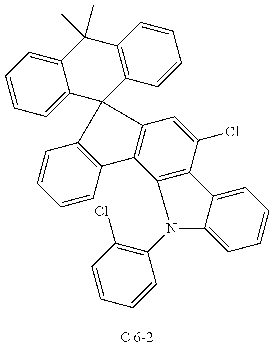 [SMILES: CC1(C)c2ccccc2C2(c3ccccc3-c3c2cc(Cl)c2c4ccccc4n(-c4ccccc4Cl)c32)c2ccccc21] | 66.3 |

### 6. Synthesis of an Intermediate Y 7-X

### Synthesis of an Intermediate A 7-1

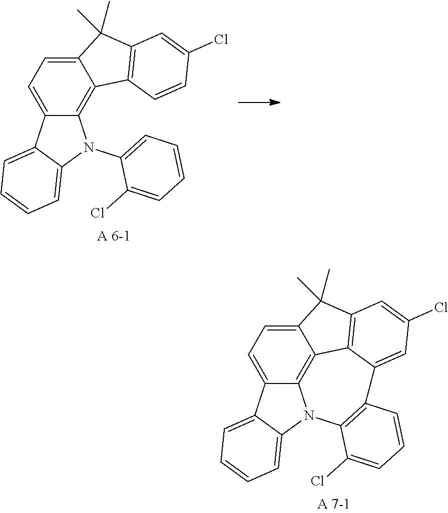 [SMILES: CC1(C)c2cc(Cl)ccc2-c2c1ccc1c3ccccc3n(-c3ccccc3Cl)c21 ; CC1(C)c2cc(Cl)cc3c2-c2c1ccc1c4ccccc4n(c21)-c1c(Cl)cccc1-3]

Nitrogen was introduced into a three-necked flask equipped with a mechanical stirrer, a thermometer, and a spherical condenser at 0.100 L/min to allow nitrogen replacement for 15 min, then the intermediate A 6-1 (32.84 g, 76.72 mmol), palladium acetate (1.71 g, 7.65 mmol), tricyclohexylphosphine fluoroborate (25.16 g, 76.72 mmol), cesium carbonate (44.92 g, 137.79 mmol), and N,N-dimethylacetamide (DMAC) (160 mL) were added, and a resulting mixture was stirred and heated to reflux for 2 h. After the reaction was completed, a resulting reaction system was cooled to room temperature; chloroform was added for extraction, a separated organic phase was dried with anhydrous magnesium sulfate, and filtered, and a filtrate was concentrated in vacuum to obtain a crude product; and the crude product was purified by silica gel column chromatography to obtain the intermediate A 7-1 (21.65 g, yield: 72.0%).

The intermediates Y 7-X (each X was an integer of 1 to 18 and each Y was A, B, or C) shown in table 14 below were each synthesized with reference to the synthesis method of the intermediate A 7-1, wherein the intermediates Y 6-X (each X was an integer of 1 to 18 and each Y was A, B, or C) were used instead of the intermediate A 6-1.

TABLE 14

| Intermediate Y 6-X | Intermediate Y 7-X | Yield (%) |
|---|---|---|
| 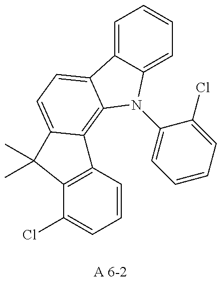 [SMILES: CC1(C)c2ccc3c4ccccc4n(-c4ccccc4Cl)c3c2-c2cccc(Cl)c21] | 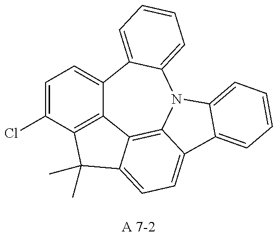 [SMILES: CC1(C)c2ccc3c4ccccc4n4c3c2-c2c(ccc(Cl)c21)-c1ccccc1-4] | 70.4 |
| 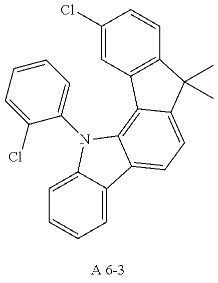 [SMILES: CC1(C)c2ccc(Cl)cc2-c2c1ccc1c3ccccc3n(-c3ccccc3Cl)c21] | 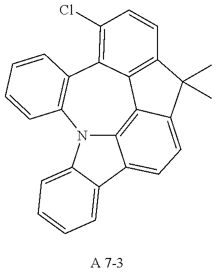 [SMILES: CC1(C)c2ccc(Cl)c3c2-c2c1ccc1c4ccccc4n(c21)-c1ccccc1-3] | 68.2 |
| 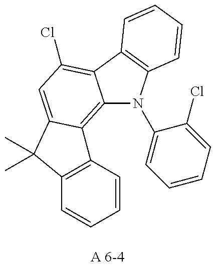 [SMILES: CC1(C)c2ccccc2-c2c1cc(Cl)c1c3ccccc3n(-c3ccccc3Cl)c21] | 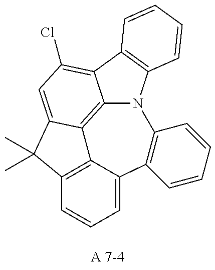 [SMILES: CC1(C)c2cccc3c2-c2c1cc(Cl)c1c4ccccc4n(c21)-c1ccccc1-3] | 69.4 |
| 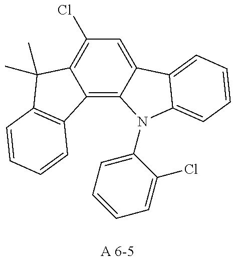 [SMILES: CC1(C)c2ccccc2-c2c1c(Cl)cc1c3ccccc3n(-c3ccccc3Cl)c21] | 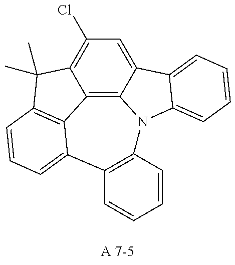 [SMILES: CC1(C)c2cccc3c2-c2c1c(Cl)cc1c4ccccc4n(c21)-c1ccccc1-3] | 67.2 |
| 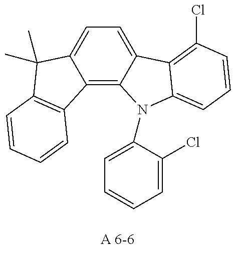 [SMILES: CC1(C)c2ccccc2-c2c1ccc1c3c(Cl)cccc3n(-c3ccccc3Cl)c21] | 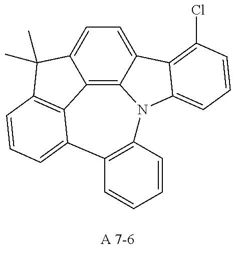 [SMILES: CC1(C)c2cccc3c2-c2c1ccc1c4c(Cl)cccc4n(c21)-c1ccccc1-3] | 70.1 |
| 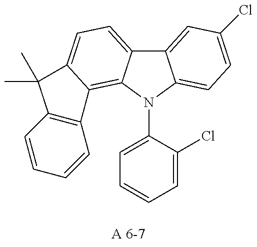 [SMILES: CC1(C)c2ccccc2-c2c1ccc1c3cc(Cl)ccc3n(-c3ccccc3Cl)c21] | 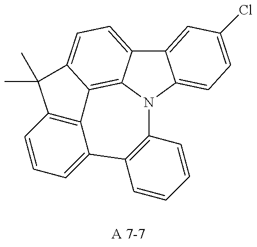 [SMILES: CC1(C)c2cccc3c2-c2c1ccc1c4cc(Cl)ccc4n(c21)-c1ccccc1-3] | 73.5 |
| 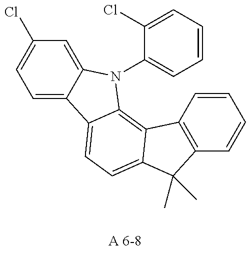 [SMILES: CC1(C)c2ccccc2-c2c1ccc1c3ccc(Cl)cc3n(-c3ccccc3Cl)c21] | 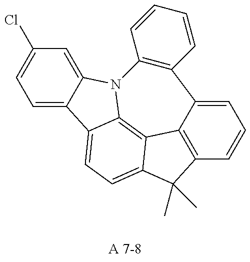 [SMILES: CC1(C)c2cccc3c2-c2c1ccc1c4ccc(Cl)cc4n(c21)-c1ccccc1-3] | 72.5 |
| 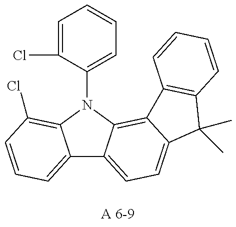 [SMILES: CC1(C)c2ccccc2-c2c1ccc1c3cccc(Cl)c3n(-c3ccccc3Cl)c21] | 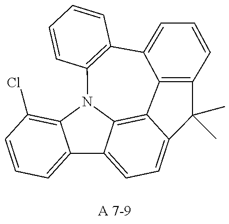 [SMILES: CC1(C)c2cccc3c2-c2c1ccc1c4cccc(Cl)c4n(c21)-c1ccccc1-3] | 68.2 |
| 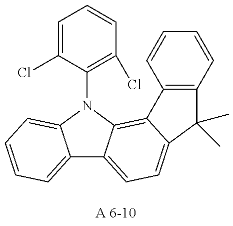 [SMILES: CC1(C)c2ccccc2-c2c1ccc1c3ccccc3n(-c3c(Cl)cccc3Cl)c21] | 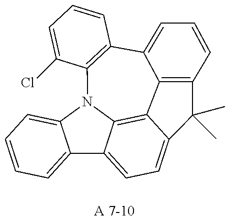 [SMILES: CC1(C)c2cccc3c2-c2c1ccc1c4ccccc4n(c21)-c1c(Cl)cccc1-3] | 66.3 |
| 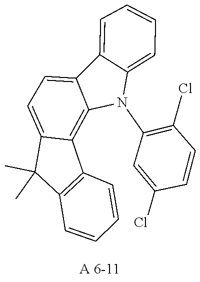 [SMILES: CC1(C)c2ccccc2-c2c1ccc1c3ccccc3n(-c3cc(Cl)ccc3Cl)c21] | 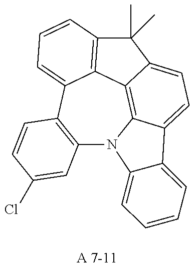 [SMILES: CC1(C)c2cccc3c2-c2c1ccc1c4ccccc4n(c21)-c1cc(Cl)ccc1-3] | 69.2 |
| 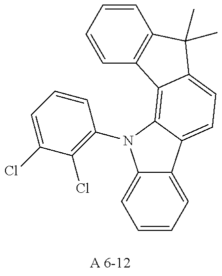 [SMILES: CC1(C)c2ccccc2-c2c1ccc1c3ccccc3n(-c3cccc(Cl)c3Cl)c21] | 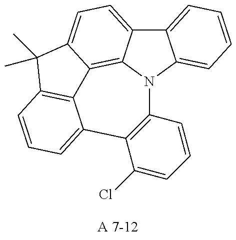 [SMILES: CC1(C)c2cccc3c2-c2c1ccc1c4ccccc4n(c21)-c1cccc(Cl)c1-3] | 67.1 |
| 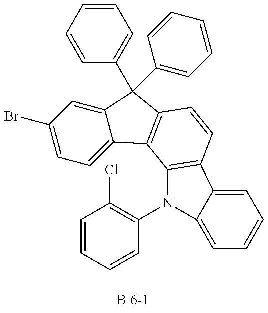 [SMILES: Clc1ccccc1-n1c2ccccc2c2ccc3c(c21)-c1ccc(Br)cc1C3(c1ccccc1)c1ccccc1] | 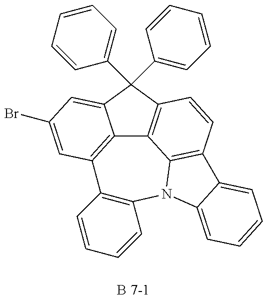 [SMILES: Brc1cc2c3c(c1)C(c1ccccc1)(c1ccccc1)c1ccc4c5ccccc5n(c4c1-3)-c1ccccc1-2] | 56.3 |
| 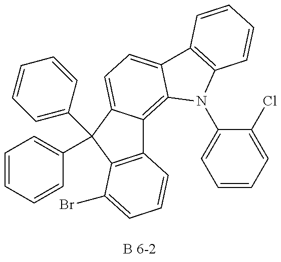 [SMILES: Clc1ccccc1-n1c2ccccc2c2ccc3c(c21)-c1cccc(Br)c1C3(c1ccccc1)c1ccccc1] | 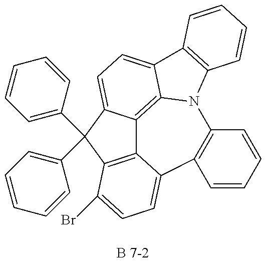 [SMILES: Brc1ccc2c3c1C(c1ccccc1)(c1ccccc1)c1ccc4c5ccccc5n(c4c1-3)-c1ccccc1-2] | 54.4 |
| 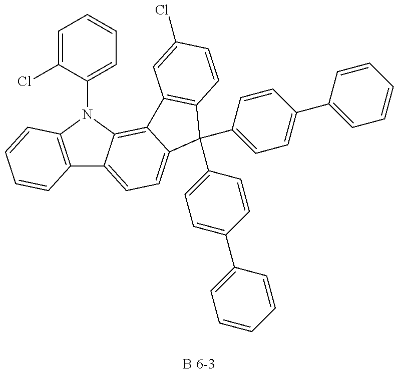 [SMILES: Clc1ccc2c(c1)-c1c(ccc3c4ccccc4n(-c4ccccc4Cl)c13)C2(c1ccc(-c2ccccc2)cc1)c1ccc(-c2ccccc2)cc1] | 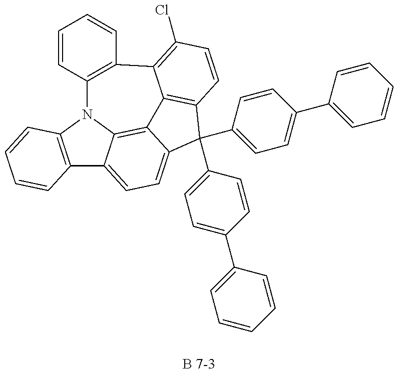 [SMILES: Clc1ccc2c3c1-c1ccccc1-n1c4ccccc4c4ccc(c-3c41)C2(c1ccc(-c2ccccc2)cc1)c1ccc(-c2ccccc2)cc1] | 55.3 |
| 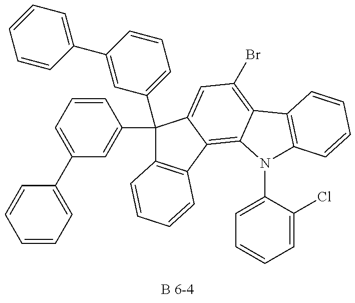 [SMILES: Clc1ccccc1-n1c2ccccc2c2c(Br)cc3c(c21)-c1ccccc1C3(c1cccc(-c2ccccc2)c1)c1cccc(-c2ccccc2)c1] | 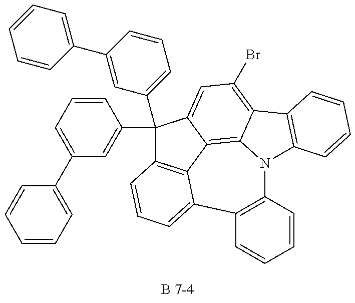 [SMILES: Brc1cc2c3c4c1c1ccccc1n4-c1ccccc1-c1cccc(c1-3)C2(c1cccc(-c2ccccc2)c1)c1cccc(-c2ccccc2)c1] | 54.2 |
| 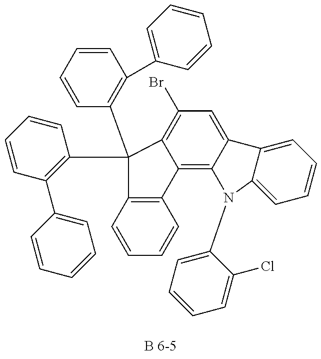 [SMILES: Clc1ccccc1-n1c2ccccc2c2cc(Br)c3c(c21)-c1ccccc1C3(c1ccccc1-c1ccccc1)c1ccccc1-c1ccccc1] | 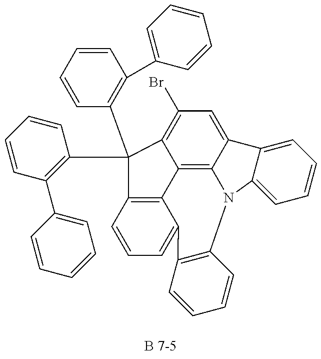 [SMILES: Brc1cc2c3ccccc3n3c2c2c1C(c1ccccc1-c1ccccc1)(c1ccccc1-c1ccccc1)c1cccc(c1-2)-c1ccccc1-3] | 56.1 |
| 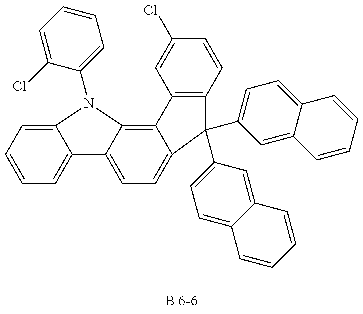 [SMILES: Clc1ccc2c(c1)-c1c(ccc3c4ccccc4n(-c4ccccc4Cl)c13)C2(c1ccc2ccccc2c1)c1ccc2ccccc2c1] | 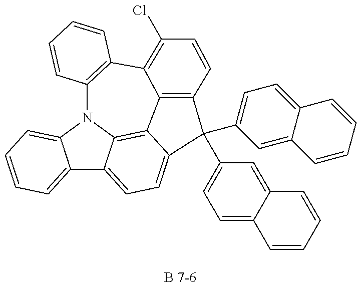 [SMILES: Clc1ccc2c3c1-c1ccccc1-n1c4ccccc4c4ccc(c-3c41)C2(c1ccc2ccccc2c1)c1ccc2ccccc2c1] | 50.3 |
| 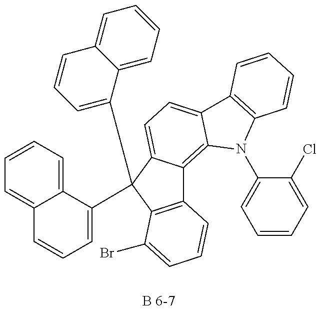 [SMILES: Clc1ccccc1-n1c2ccccc2c2ccc3c(c21)-c1cccc(Br)c1C3(c1cccc2ccccc12)c1cccc2ccccc12] | 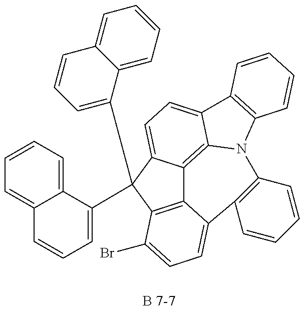 [SMILES: Brc1ccc2c3c1C(c1cccc4ccccc14)(c1cccc4ccccc14)c1ccc4c5ccccc5n(c4c1-3)-c1ccccc1-2] | 51.2 |
| 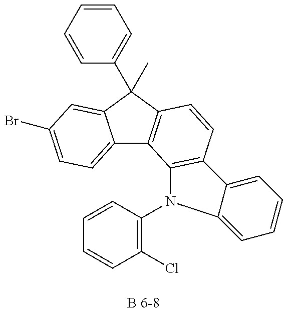 [SMILES: CC1(c2ccccc2)c2cc(Br)ccc2-c2c1ccc1c3ccccc3n(-c3ccccc3Cl)c21] | 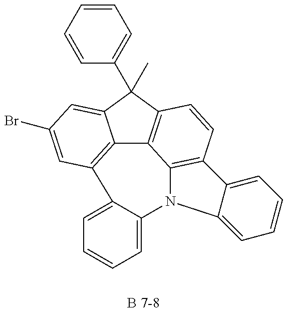 [SMILES: CC1(c2ccccc2)c2cc(Br)cc3c2-c2c1ccc1c4ccccc4n(c21)-c1ccccc1-3] | 55.5 |
| 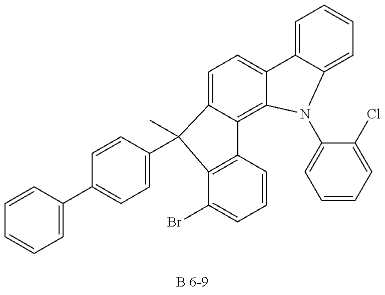 [SMILES: CC1(c2ccc(-c3ccccc3)cc2)c2ccc3c4ccccc4n(-c4ccccc4Cl)c3c2-c2cccc(Br)c21] | 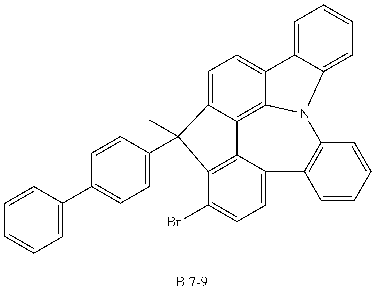 [SMILES: CC1(c2ccc(-c3ccccc3)cc2)c2ccc3c4ccccc4n4c3c2-c2c(ccc(Br)c21)-c1ccccc1-4] | 56.3 |
| 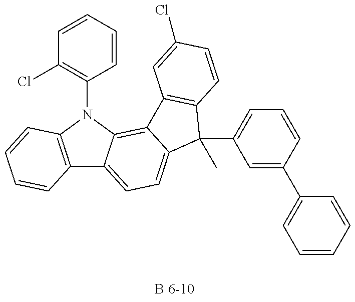 [SMILES: CC1(c2cccc(-c3ccccc3)c2)c2ccc(Cl)cc2-c2c1ccc1c3ccccc3n(-c3ccccc3Cl)c21] | 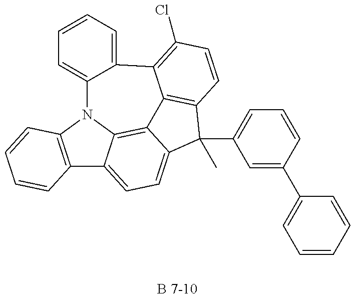 [SMILES: CC1(c2cccc(-c3ccccc3)c2)c2ccc(Cl)c3c2-c2c1ccc1c4ccccc4n(c21)-c1ccccc1-3] | 51.4 |
| 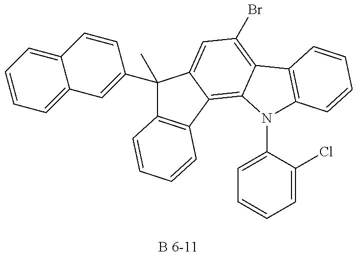 [SMILES: CC1(c2ccc3ccccc3c2)c2ccccc2-c2c1cc(Br)c1c3ccccc3n(-c3ccccc3Cl)c21] | 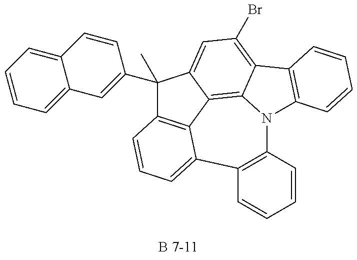 [SMILES: CC1(c2ccc3ccccc3c2)c2cccc3c2-c2c1cc(Br)c1c4ccccc4n(c21)-c1ccccc1-3] | 56.2 |
| 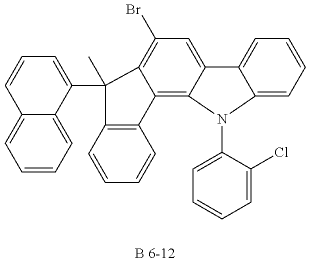 [SMILES: CC1(c2cccc3ccccc23)c2ccccc2-c2c1c(Br)cc1c3ccccc3n(-c3ccccc3Cl)c21] | 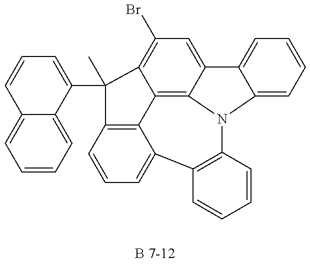 [SMILES: CC1(c2cccc3ccccc23)c2cccc3c2-c2c1c(Br)cc1c4ccccc4n(c21)-c1ccccc1-3] | 53.6 |
| 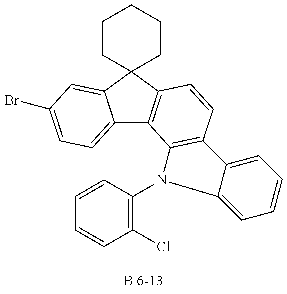 [SMILES: Clc1ccccc1-n1c2ccccc2c2ccc3c(c21)-c1ccc(Br)cc1C31CCCCC1] | 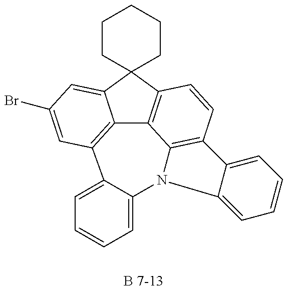 [SMILES: Brc1cc2c3c(c1)C1(CCCCC1)c1ccc4c5ccccc5n(c4c1-3)-c1ccccc1-2] | 56.4 |
| 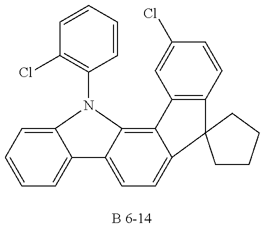 [SMILES: Clc1ccc2c(c1)-c1c(ccc3c4ccccc4n(-c4ccccc4Cl)c13)C21CCCC1] | 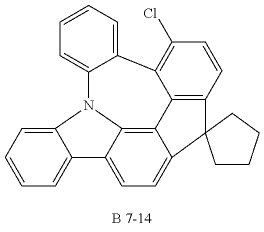 [SMILES: Clc1ccc2c3c1-c1ccccc1-n1c4ccccc4c4ccc(c-3c41)C21CCCC1] | 55.2 |
| 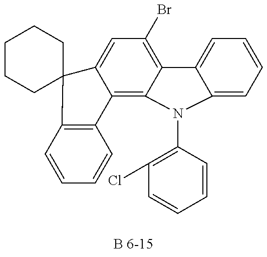 [SMILES: Clc1ccccc1-n1c2ccccc2c2c(Br)cc3c(c21)-c1ccccc1C31CCCCC1] | 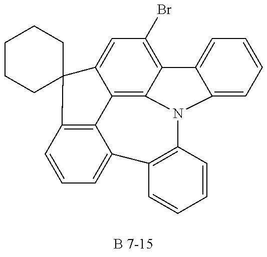 [SMILES: Brc1cc2c3c4c1c1ccccc1n4-c1ccccc1-c1cccc(c1-3)C21CCCCC1] | 56.6 |
| 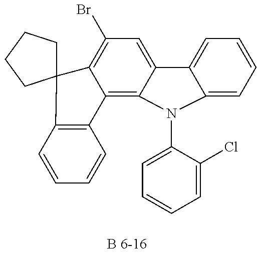 [SMILES: Clc1ccccc1-n1c2ccccc2c2cc(Br)c3c(c21)-c1ccccc1C31CCCC1] | 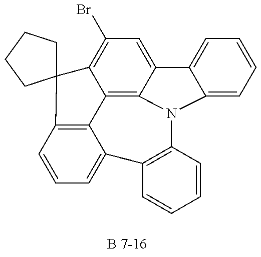 [SMILES: Brc1cc2c3ccccc3n3c2c2c1C1(CCCC1)c1cccc(c1-2)-c1ccccc1-3] | 51.1 |
| 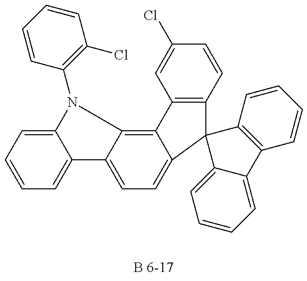 [SMILES: Clc1ccc2c(c1)-c1c(ccc3c4ccccc4n(-c4ccccc4Cl)c13)C21c2ccccc2-c2ccccc21] | 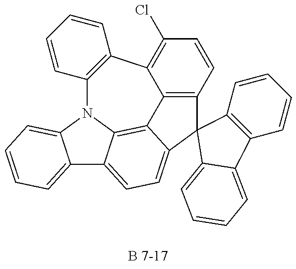 [SMILES: Clc1ccc2c3c1-c1ccccc1-n1c4ccccc4c4ccc(c-3c41)C21c2ccccc2-c2ccccc21] | 50.5 |
| 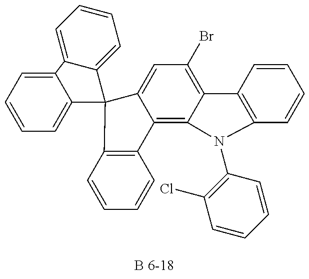 [SMILES: Clc1ccccc1-n1c2ccccc2c2c(Br)cc3c(c21)-c1ccccc1C31c2ccccc2-c2ccccc21] | 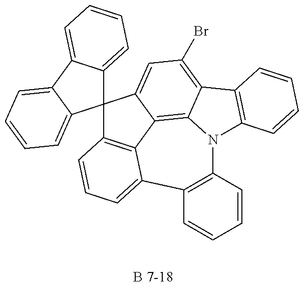 [SMILES: Brc1cc2c3c4c1c1ccccc1n4-c1ccccc1-c1cccc(c1-3)C21c2ccccc2-c2ccccc21] | 56.3 |
| 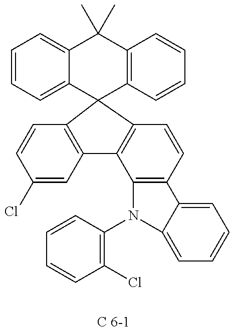 [SMILES: CC1(C)c2ccccc2C2(c3ccc(Cl)cc3-c3c2ccc2c4ccccc4n(-c4ccccc4Cl)c32)c2ccccc21] | 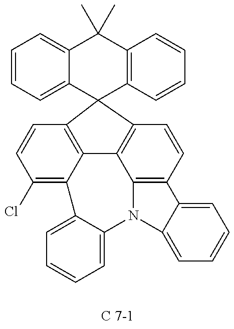 [SMILES: CC1(C)c2ccccc2C2(c3ccccc31)c1ccc(Cl)c3c1-c1c2ccc2c4ccccc4n(c12)-c1ccccc1-3] | 52.1 |
| 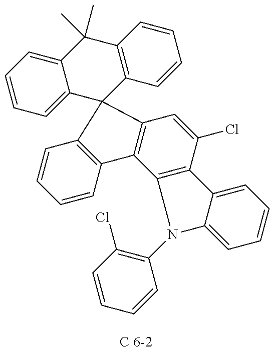 [SMILES: CC1(C)c2ccccc2C2(c3ccccc3-c3c2cc(Cl)c2c4ccccc4n(-c4ccccc4Cl)c32)c2ccccc21] | 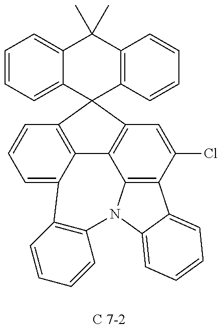 [SMILES: CC1(C)c2ccccc2C2(c3ccccc31)c1cccc3c1-c1c2cc(Cl)c2c4ccccc4n(c12)-c1ccccc1-3] | 56.8 |

### 7. Synthesis of an Intermediate Y 9-X

### Synthesis of an Intermediate A 9-1

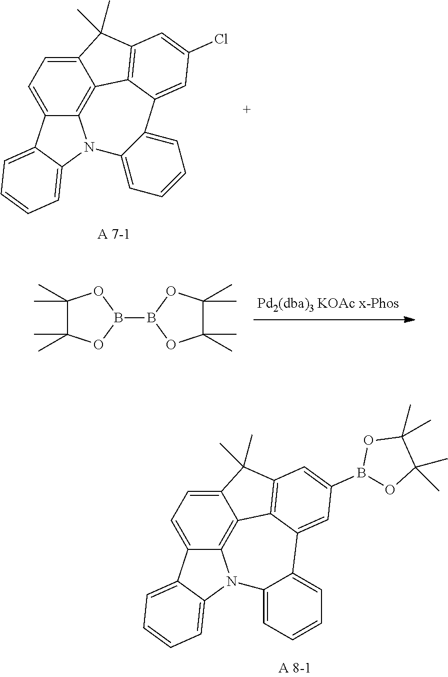

Nitrogen was introduced into a three-necked flask equipped with a mechanical stirrer, a thermometer, and a spherical condenser at 0.100 L/min to allow nitrogen replacement for 15 min, then the intermediate A 7-1 (20.3 g, 51.79 mmol), bis(pinacolato)diboron (13.1 g, 51.79 mmol), potassium acetate (7.62 g, 77.68 mmol), 2-dicyclohexylphosphino-2,4,6-triisopropylbiphenyl (x-Phos) (0.49 g, 1.036 mmol), and tris(dibenzylideneacetone)dipalladium (0.47 g, 0.518 mmol), and 1,4-dioxane (160 mL) were added, and a resulting mixture was heated to reflux at 75° C. to 85° C. and stirred for 3 h. After the reaction was completed, a resulting reaction system was cooled to room temperature; the reaction solution was subjected to extraction, a separated organic phase was dried with anhydrous magnesium sulfate and filtered, and a filtrate was concentrated in vacuum to obtain a crude product; and the crude product was purified by recrystallization with a toluene system, and a resulting precipitate was filtered out to obtain the intermediate A 8-1 (25.01 g, yield: 76.1%).

The intermediates Y 8-X (each X was an integer of 1 to 14 or 17 and each Y was A, B, or C) shown in table 15 below were each synthesized with reference to the synthesis method of the intermediate A 8-1, wherein the intermediates Y 7-X (each X was an integer of 1 to 14 or 17 and each Y was A, B, or C) were used instead of the intermediate A 7-1.

TABLE 15

| Intermediate Y 7-X | Intermediate Y 8-X | Yield (%) |
|---|---|---|
| 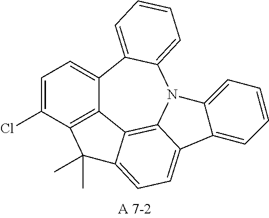 [SMILES: CC1(C)c2ccc3c4ccccc4n4c3c2-c2c(ccc(Cl)c21)-c1ccccc1-4] | 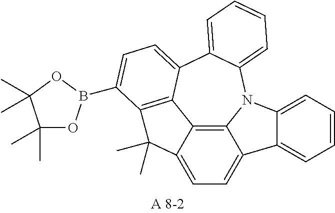 [SMILES: CC1(C)c2ccc3c4ccccc4n4c3c2-c2c(ccc(B3OC(C)(C)C(C)(C)O3)c21)-c1ccccc1-4] | 70.3 |
| 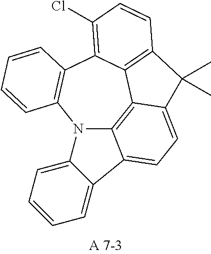 [SMILES: CC1(C)c2ccc(Cl)c3c2-c2c1ccc1c4ccccc4n(c21)-c1ccccc1-3] | 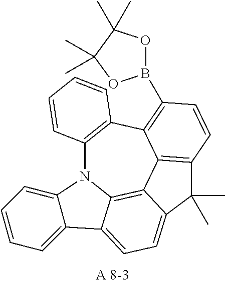 [SMILES: CC1(C)c2ccc(B3OC(C)(C)C(C)(C)O3)c3c2-c2c1ccc1c4ccccc4n(c21)-c1ccccc1-3] | 71.4 |
| 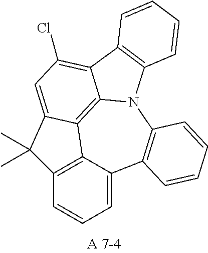 [SMILES: CC1(C)c2cccc3c2-c2c1cc(Cl)c1c4ccccc4n(c21)-c1ccccc1-3] | 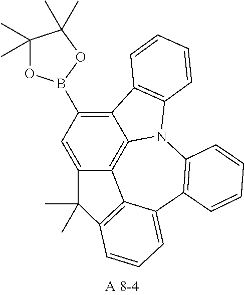 [SMILES: CC1(C)c2cccc3c2-c2c1cc(B1OC(C)(C)C(C)(C)O1)c1c4ccccc4n(c21)-c1ccccc1-3] | 72.2 |
| 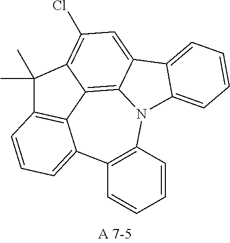 [SMILES: CC1(C)c2cccc3c2-c2c1c(Cl)cc1c4ccccc4n(c21)-c1ccccc1-3] | 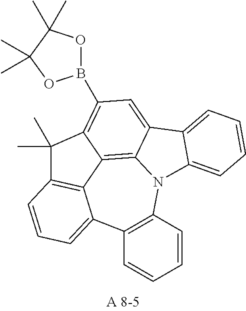 [SMILES: CC1(C)c2cccc3c2-c2c1c(B1OC(C)(C)C(C)(C)O1)cc1c4ccccc4n(c21)-c1ccccc1-3] | 69.1 |
| 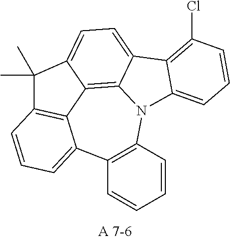 [SMILES: CC1(C)c2cccc3c2-c2c1ccc1c4c(Cl)cccc4n(c21)-c1ccccc1-3] | 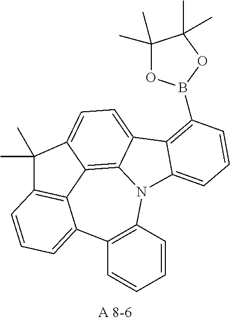 [SMILES: CC1(C)c2cccc3c2-c2c1ccc1c4c(B5OC(C)(C)C(C)(C)O5)cccc4n(c21)-c1ccccc1-3] | 70.6 |
| 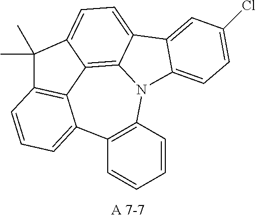 [SMILES: CC1(C)c2cccc3c2-c2c1ccc1c4cc(Cl)ccc4n(c21)-c1ccccc1-3] | 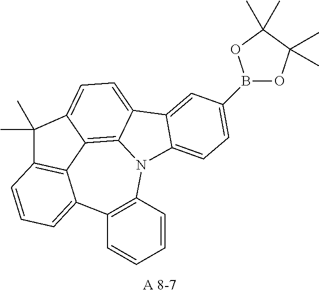 | 73.5 |
| 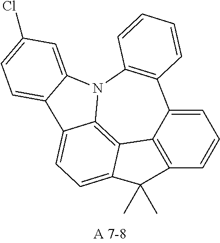 [SMILES: CC1(C)c2cccc3c2-c2c1ccc1c4ccc(Cl)cc4n(c21)-c1ccccc1-3] | 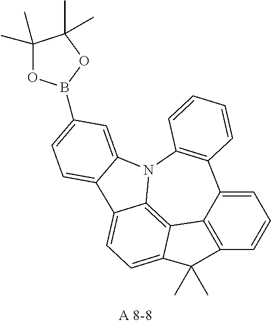 [SMILES: CC1(C)c2cccc3c2-c2c1ccc1c4ccc(B5OC(C)(C)C(C)(C)O5)cc4n(c21)-c1ccccc1-3] | 74.1 |
| 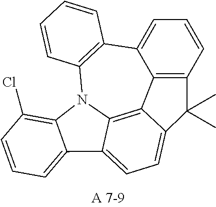 [SMILES: CC1(C)c2cccc3c2-c2c1ccc1c4cccc(Cl)c4n(c21)-c1ccccc1-3] | 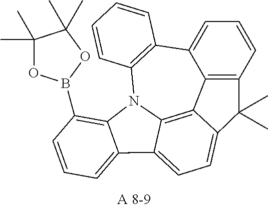 | 70.4 |
| 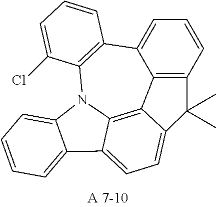 [SMILES: CC1(C)c2cccc3c2-c2c1ccc1c4ccccc4n(c21)-c1c(Cl)cccc1-3] | 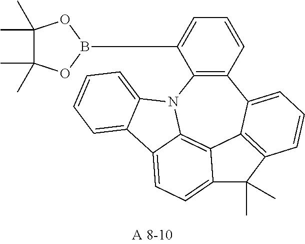 [SMILES: CC1(C)c2cccc3c2-c2c1ccc1c4ccccc4n(c21)-c1c(B2OC(C)(C)C(C)(C)O2)cccc1-3] | 66.2 |
| 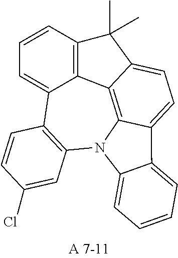 [SMILES: CC1(C)c2cccc3c2-c2c1ccc1c4ccccc4n(c21)-c1cc(Cl)ccc1-3] | 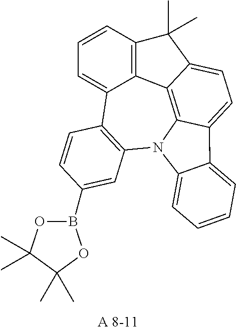 [SMILES: CC1(C)c2cccc3c2-c2c1ccc1c4ccccc4n(c21)-c1cc(B2OC(C)(C)C(C)(C)O2)ccc1-3] | 71.3 |
| 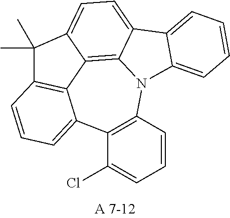 [SMILES: CC1(C)c2cccc3c2-c2c1ccc1c4ccccc4n(c21)-c1cccc(Cl)c1-3] | 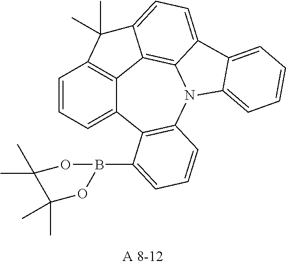 [SMILES: CC1(C)c2cccc3c2-c2c1ccc1c4ccccc4n(c21)-c1cccc(B2OC(C)(C)C(C)(C)O2)c1-3] | 68.5 |
| 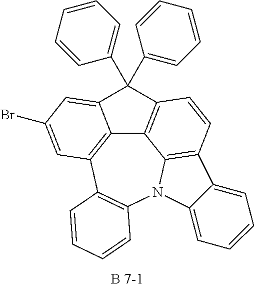 [SMILES: Brc1cc2c3c(c1)C(c1ccccc1)(c1ccccc1)c1ccc4c5ccccc5n(c4c1-3)-c1ccccc1-2] | 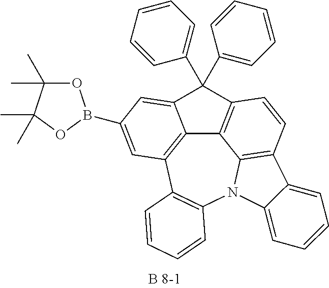 [SMILES: CC1(C)OB(c2cc3c4c(c2)C(c2ccccc2)(c2ccccc2)c2ccc5c6ccccc6n(c5c2-4)-c2ccccc2-3)OC1(C)C] | 70.1 |
| 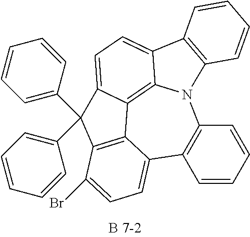 [SMILES: Brc1ccc2c3c1C(c1ccccc1)(c1ccccc1)c1ccc4c5ccccc5n(c4c1-3)-c1ccccc1-2] | 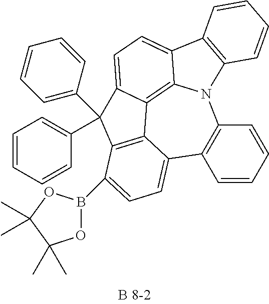 [SMILES: CC1(C)OB(c2ccc3c4c2C(c2ccccc2)(c2ccccc2)c2ccc5c6ccccc6n(c5c2-4)-c2ccccc2-3)OC1(C)C] | 64.2 |
| 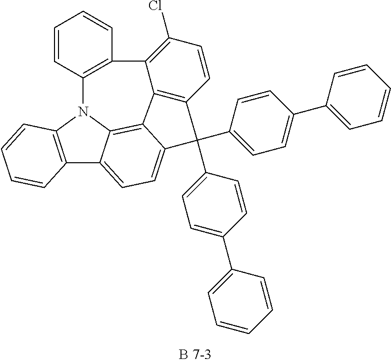 [SMILES: Clc1ccc2c3c1-c1ccccc1-n1c4ccccc4c4ccc(c-3c41)C2(c1ccc(-c2ccccc2)cc1)c1ccc(-c2ccccc2)cc1] | 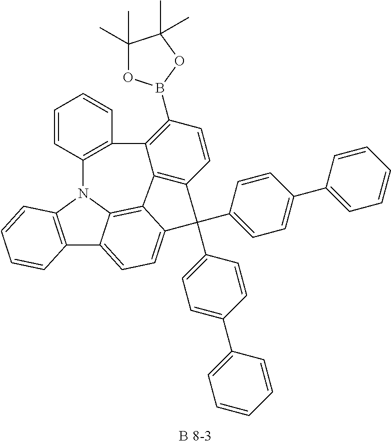 [SMILES: CC1(C)OB(c2ccc3c4c2-c2ccccc2-n2c5ccccc5c5ccc(c-4c52)C3(c2ccc(-c3ccccc3)cc2)c2ccc(-c3ccccc3)cc2)OC1(C)C] | 65.4 |
| 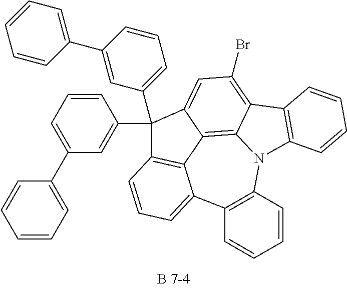 [SMILES: Brc1cc2c3c4c1c1ccccc1n4-c1ccccc1-c1cccc(c1-3)C2(c1cccc(-c2ccccc2)c1)c1cccc(-c2ccccc2)c1] | 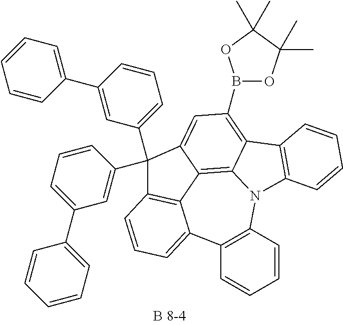 [SMILES: CC1(C)OB(c2cc3c4c5c2c2ccccc2n5-c2ccccc2-c2cccc(c2-4)C3(c2cccc(-c3ccccc3)c2)c2cccc(-c3ccccc3)c2)OC1(C)C] | 68.3 |
| 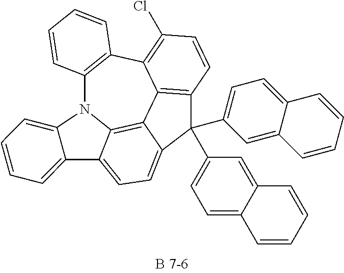 [SMILES: Clc1ccc2c3c1-c1ccccc1-n1c4ccccc4c4ccc(c-3c41)C2(c1ccc2ccccc2c1)c1ccc2ccccc2c1] | 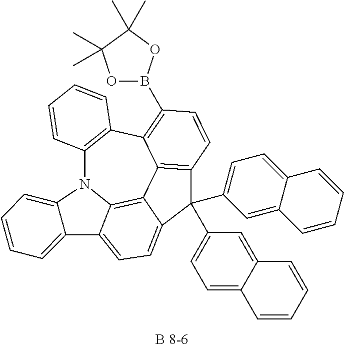 [SMILES: CC1(C)OB(c2ccc3c4c2-c2ccccc2-n2c5ccccc5c5ccc(c-4c52)C3(c2ccc3ccccc3c2)c2ccc3ccccc3c2)OC1(C)C] | 64.5 |
| 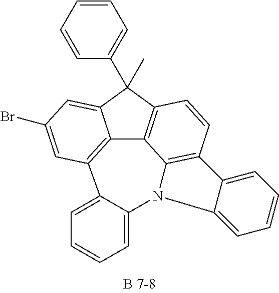 [SMILES: CC1(c2ccccc2)c2cc(Br)cc3c2-c2c1ccc1c4ccccc4n(c21)-c1ccccc1-3] | 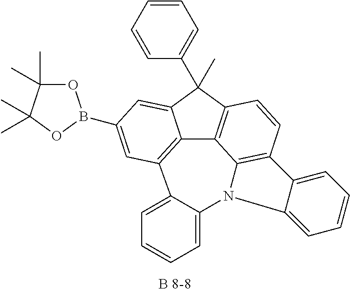 [SMILES: CC1(c2ccccc2)c2cc(B3OC(C)(C)C(C)(C)O3)cc3c2-c2c1ccc1c4ccccc4n(c21)-c1ccccc1-3] | 69.0 |
| 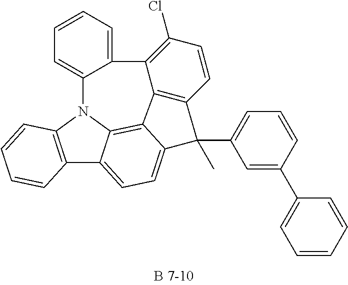 [SMILES: CC1(c2cccc(-c3ccccc3)c2)c2ccc(Cl)c3c2-c2c1ccc1c4ccccc4n(c21)-c1ccccc1-3] | 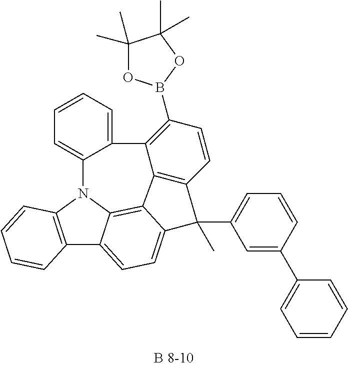 [SMILES: CC1(c2cccc(-c3ccccc3)c2)c2ccc(B3OC(C)(C)C(C)(C)O3)c3c2-c2c1ccc1c4ccccc4n(c21)-c1ccccc1-3] | 62.3 |
| 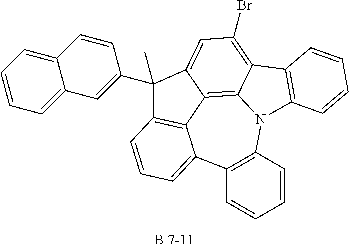 [SMILES: CC1(c2ccc3ccccc3c2)c2cccc3c2-c2c1cc(Br)c1c4ccccc4n(c21)-c1ccccc1-3] | 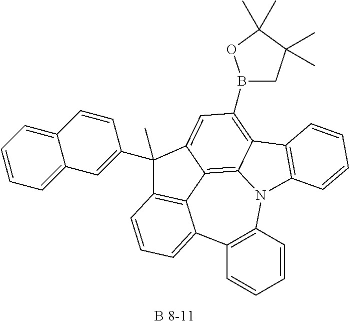 [SMILES: CC1(c2ccc3ccccc3c2)c2cccc3c2-c2c1cc(B1CC(C)(C)C(C)(C)O1)c1c4ccccc4n(c21)-c1ccccc1-3] | 66.1 |
| 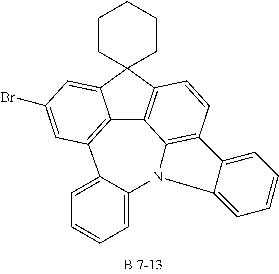 [SMILES: Brc1cc2c3c(c1)C1(CCCCC1)c1ccc4c5ccccc5n(c4c1-3)-c1ccccc1-2] | 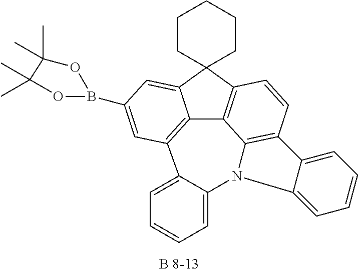 [SMILES: CC1(C)OB(c2cc3c4c(c2)C2(CCCCC2)c2ccc5c6ccccc6n(c5c2-4)-c2ccccc2-3)OC1(C)C] | 67.2 |
| 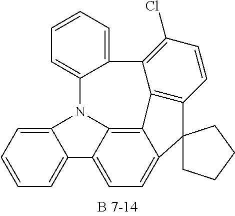 [SMILES: Clc1ccc2c3c1-c1ccccc1-n1c4ccccc4c4ccc(c-3c41)C21CCCC1] | 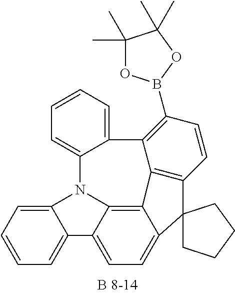 [SMILES: CC1(C)OB(c2ccc3c4c2-c2ccccc2-n2c5ccccc5c5ccc(c-4c52)C32CCCC2)OC1(C)C] | 62.6 |
| 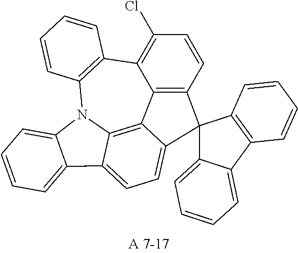 [SMILES: Clc1ccc2c3c1-c1ccccc1-n1c4ccccc4c4ccc(c-3c41)C21c2ccccc2-c2ccccc21] | 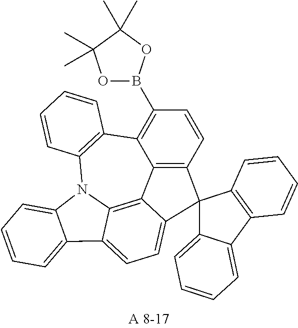 [SMILES: CC1(C)OB(c2ccc3c4c2-c2ccccc2-n2c5ccccc5c5ccc(c-4c52)C32c3ccccc3-c3ccccc32)OC1(C)C] | 61.1 |
| 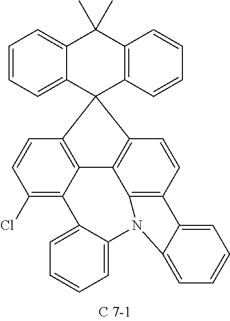 [SMILES: CC1(C)c2ccccc2C2(c3ccccc31)c1ccc(Cl)c3c1-c1c2ccc2c4ccccc4n(c12)-c1ccccc1-3] | 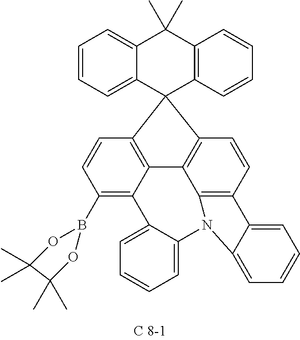 [SMILES: CC1(C)c2ccccc2C2(c3ccccc31)c1ccc(B3OC(C)(C)C(C)(C)O3)c3c1-c1c2ccc2c4ccccc4n(c12)-c1ccccc1-3] | 62.5 |

### 8. Synthesis of an Intermediate Y 10-X

### Synthesis of an Intermediate A 10-1

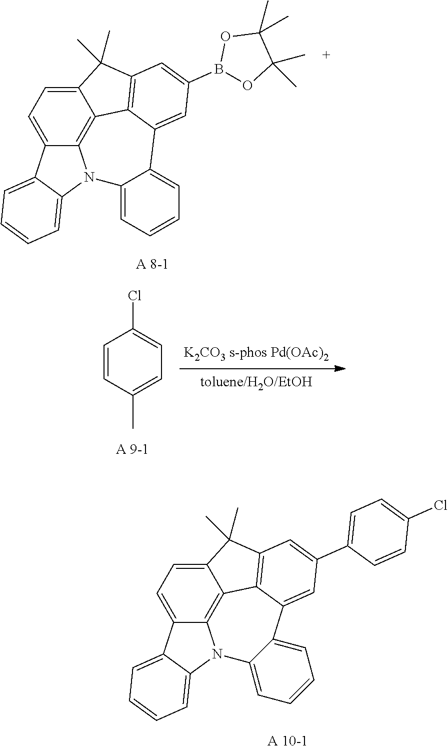

Nitrogen was introduced into a three-necked flask equipped with a mechanical stirrer, a thermometer, and a spherical condenser at 0.100 L/min to allow nitrogen replacement for 15 min, then the intermediate A 8-1 (18.44 g, 38.18 mmol), a reactant A 9-1 (9.09 g, 38.18 mmol), palladium acetate (0.124 g, 0.382 mmol), potassium carbonate (7.9 g, 57.27 mmol), 2-dicyclohexylphosphino-2′,6′-dimethoxybiphenyl (s-phos) (0.313 g, 0.7636 mmol), toluene (108 mL), absolute ethanol (36 mL), and deionized water (36 mL) were added, and a resulting mixture was heated to reflux at 70° C. to 80° C. and stirred for 4 h. After the reaction was completed, a resulting reaction system was cooled to room temperature; toluene was added for extraction, the combined organic phases were washed with water, and then dried with anhydrous magnesium sulfate, and filtered, and a filtrate was concentrated in vacuum to obtain a crude product; and the crude product was purified by recrystallization with a mixture of DCM and n-heptane system to obtain a solid intermediate A 10-1 (13.4 g, yield: 75.1%).

The intermediates Y 10-X (each X was an integer of 1 to 14 or 17 and each Y was A, B, or C) shown in table 16 below were each synthesized with reference to the synthesis method of the intermediate A 10-1, wherein the intermediates Y 8-X (each X was an integer of 1 to 14 or 17 and each Y was A, B, or C) were used instead of the intermediate A 8-1 and the reactants A 9-X (each X was an integer of 1 to 10) were used instead of the reactant A 9-1.

TABLE 16

| Intermediate Y 8-X | Reactant A 9-X |
|---|---|
| 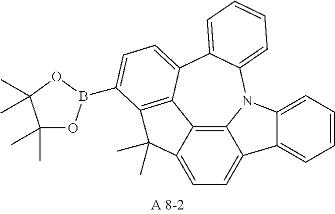 [SMILES: CC1(C)c2ccc3c4ccccc4n4c3c2-c2c(ccc(B3OC(C)(C)C(C)(C)O3)c21)-c1ccccc1-4] | 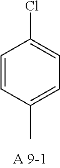 |
| 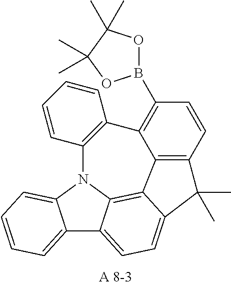 [SMILES: CC1(C)c2ccc(B3OC(C)(C)C(C)(C)O3)c3c2-c2c1ccc1c4ccccc4n(c21)-c1ccccc1-3] | 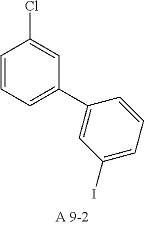 [SMILES: Clc1cccc(-c2cccc(I)c2)c1] |
| 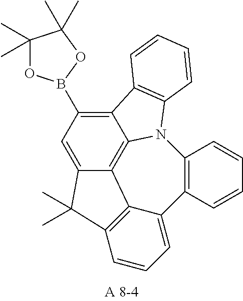 [SMILES: CC1(C)c2cccc3c2-c2c1cc(B1OC(C)(C)C(C)(C)O1)c1c4ccccc4n(c21)-c1ccccc1-3] | 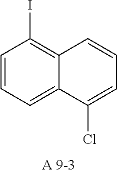 [SMILES: Clc1cccc2c(I)cccc12] |
| 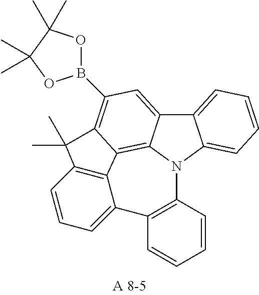 [SMILES: CC1(C)c2cccc3c2-c2c1c(B1OC(C)(C)C(C)(C)O1)cc1c4ccccc4n(c21)-c1ccccc1-3] | 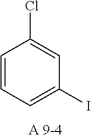 |
| 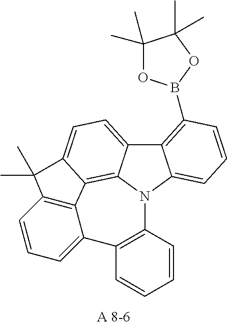 [SMILES: CC1(C)c2cccc3c2-c2c1ccc1c4c(B5OC(C)(C)C(C)(C)O5)cccc4n(c21)-c1ccccc1-3] | 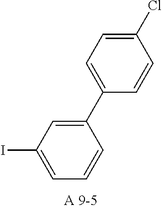 [SMILES: Clc1ccc(-c2cccc(I)c2)cc1] |
| 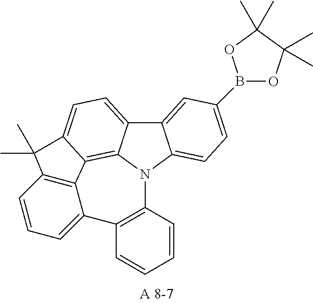 | 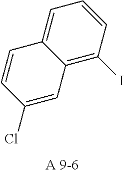 [SMILES: Clc1ccc2cccc(I)c2c1] |
| 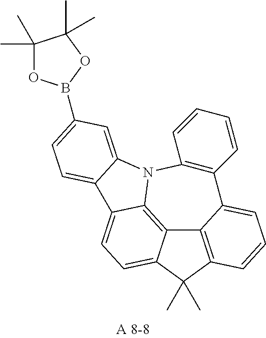 [SMILES: CC1(C)c2cccc3c2-c2c1ccc1c4ccc(B5OC(C)(C)C(C)(C)O5)cc4n(c21)-c1ccccc1-3] | 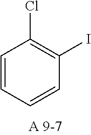 |
| 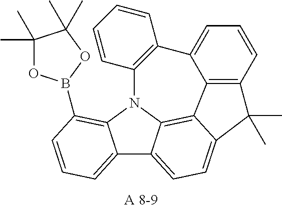 | 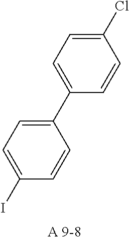 [SMILES: Clc1ccc(-c2ccc(I)cc2)cc1] |
| 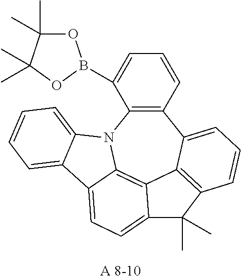 [SMILES: CC1(C)c2cccc3c2-c2c1ccc1c4ccccc4n(c21)-c1c(B2OC(C)(C)C(C)(C)O2)cccc1-3] | 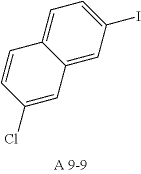 [SMILES: Clc1ccc2ccc(I)cc2c1] |
| 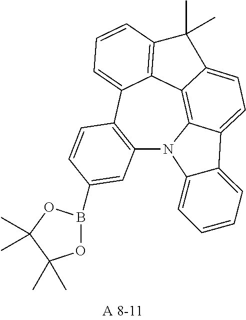 [SMILES: CC1(C)c2cccc3c2-c2c1ccc1c4ccccc4n(c21)-c1cc(B2OC(C)(C)C(C)(C)O2)ccc1-3] | 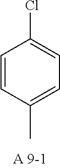 |
| 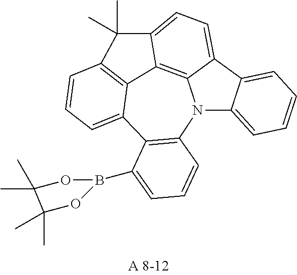 [SMILES: CC1(C)c2cccc3c2-c2c1ccc1c4ccccc4n(c21)-c1cccc(B2OC(C)(C)C(C)(C)O2)c1-3] | 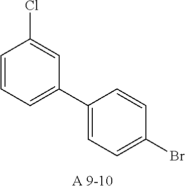 [SMILES: Clc1cccc(-c2ccc(Br)cc2)c1] |
| 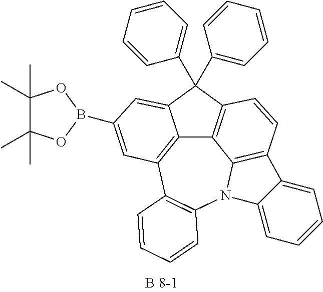 [SMILES: CC1(C)OB(c2cc3c4c(c2)C(c2ccccc2)(c2ccccc2)c2ccc5c6ccccc6n(c5c2-4)-c2ccccc2-3)OC1(C)C] | 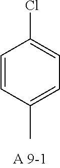 |
| 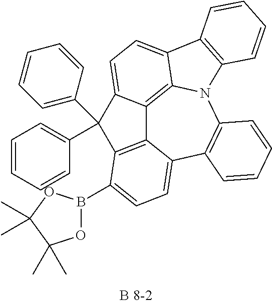 [SMILES: CC1(C)OB(c2ccc3c4c2C(c2ccccc2)(c2ccccc2)c2ccc5c6ccccc6n(c5c2-4)-c2ccccc2-3)OC1(C)C] | 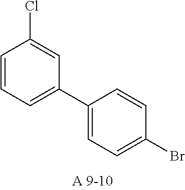 [SMILES: Clc1cccc(-c2ccc(Br)cc2)c1] |
| 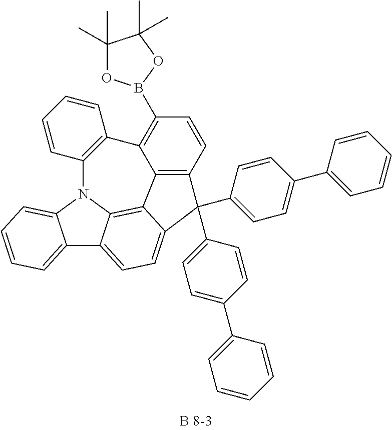 [SMILES: CC1(C)OB(c2ccc3c4c2-c2ccccc2-n2c5ccccc5c5ccc(c-4c52)C3(c2ccc(-c3ccccc3)cc2)c2ccc(-c3ccccc3)cc2)OC1(C)C] | 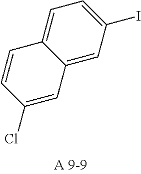 [SMILES: Clc1ccc2ccc(I)cc2c1] |
| 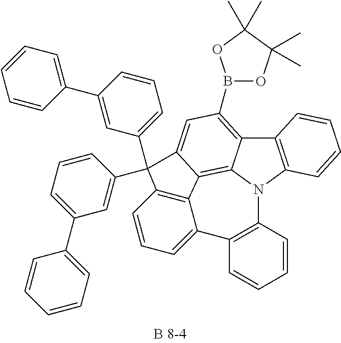 [SMILES: CC1(C)OB(c2cc3c4c5c2c2ccccc2n5-c2ccccc2-c2cccc(c2-4)C3(c2cccc(-c3ccccc3)c2)c2cccc(-c3ccccc3)c2)OC1(C)C] | 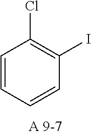 |
| 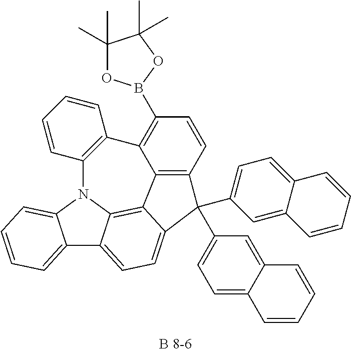 [SMILES: CC1(C)OB(c2ccc3c4c2-c2ccccc2-n2c5ccccc5c5ccc(c-4c52)C3(c2ccc3ccccc3c2)c2ccc3ccccc3c2)OC1(C)C] | 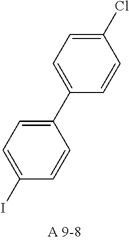 [SMILES: Clc1ccc(-c2ccc(I)cc2)cc1] |
| 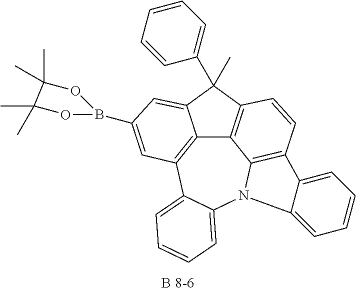 [SMILES: CC1(c2ccccc2)c2cc(B3OC(C)(C)C(C)(C)O3)cc3c2-c2c1ccc1c4ccccc4n(c21)-c1ccccc1-3] | 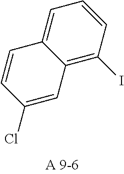 [SMILES: Clc1ccc2cccc(I)c2c1] |
| 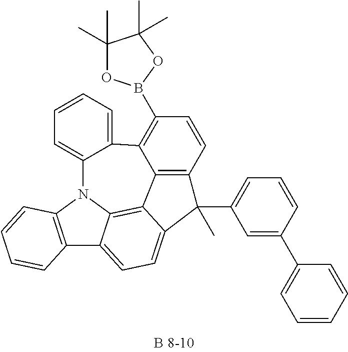 [SMILES: CC1(c2cccc(-c3ccccc3)c2)c2ccc(B3OC(C)(C)C(C)(C)O3)c3c2-c2c1ccc1c4ccccc4n(c21)-c1ccccc1-3] | 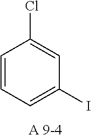 |
| 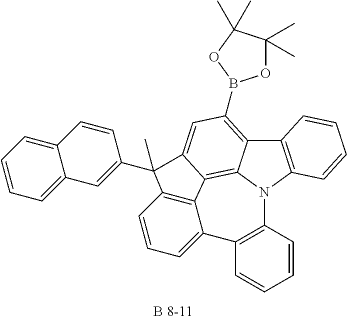 [SMILES: CC1(c2ccc3ccccc3c2)c2cccc3c2-c2c1cc(B1OC(C)(C)C(C)(C)O1)c1c4ccccc4n(c21)-c1ccccc1-3] | 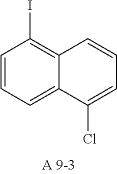 [SMILES: Clc1cccc2c(I)cccc12] |
| 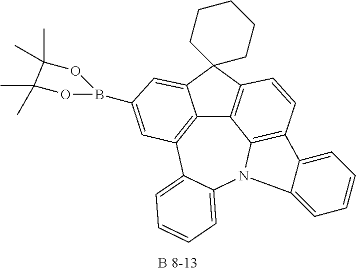 [SMILES: CC1(C)OB(c2cc3c4c(c2)C2(CCCCC2)c2ccc5c6ccccc6n(c5c2-4)-c2ccccc2-3)OC1(C)C] | 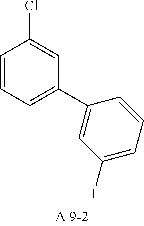 [SMILES: Clc1cccc(-c2cccc(I)c2)c1] |
| 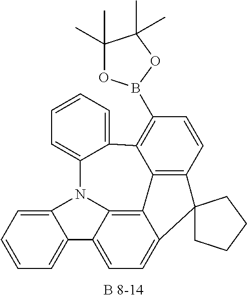 [SMILES: CC1(C)OB(c2ccc3c4c2-c2ccccc2-n2c5ccccc5c5ccc(c-4c52)C32CCCC2)OC1(C)C] | 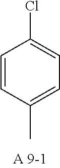 |
| 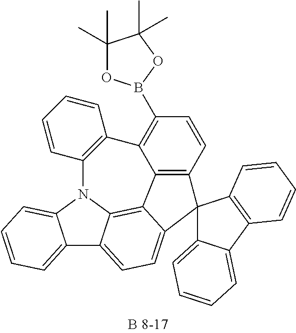 [SMILES: CC1(C)OB(c2ccc3c4c2-c2ccccc2-n2c5ccccc5c5ccc(c-4c52)C32c3ccccc3-c3ccccc32)OC1(C)C] | 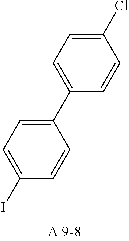 [SMILES: Clc1ccc(-c2ccc(I)cc2)cc1] |
| 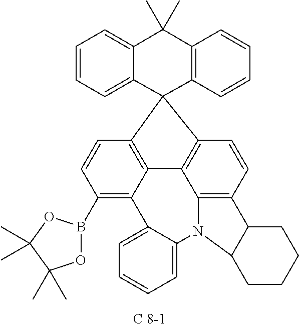 [SMILES: CC1(C)c2ccccc2C2(c3ccccc31)c1ccc(B3OC(C)(C)C(C)(C)O3)c3c1-c1c2ccc2c1N(c1ccccc1-3)C1CCCCC21] | 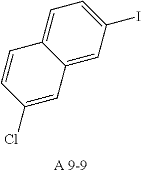 [SMILES: Clc1ccc2ccc(I)cc2c1] |
| Intermediate Y 10-X | Yield (%) |
| 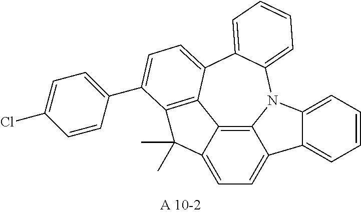 [SMILES: CC1(C)c2ccc3c4ccccc4n4c3c2-c2c(ccc(-c3ccc(Cl)cc3)c21)-c1ccccc1-4] | 70.3 |
| 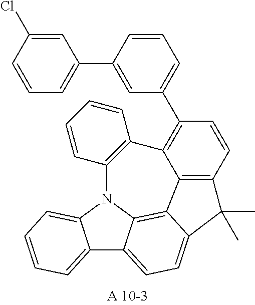 [SMILES: CC1(C)c2ccc(-c3cccc(-c4cccc(Cl)c4)c3)c3c2-c2c1ccc1c4ccccc4n(c21)-c1ccccc1-3] | 67.8 |
| 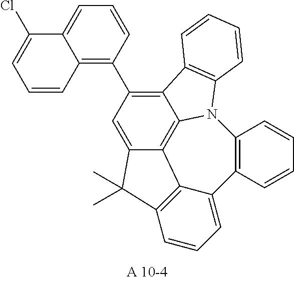 [SMILES: CC1(C)c2cccc3c2-c2c1cc(-c1cccc4c(Cl)cccc14)c1c4ccccc4n(c21)-c1ccccc1-3] | 71.2 |
| 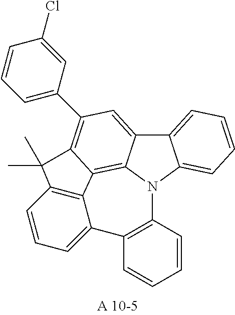 [SMILES: CC1(C)c2cccc3c2-c2c1c(-c1cccc(Cl)c1)cc1c4ccccc4n(c21)-c1ccccc1-3] | 72.5 |
| 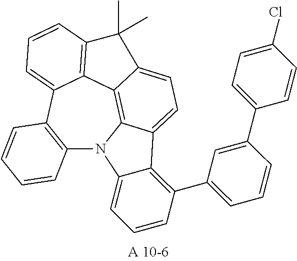 [SMILES: CC1(C)c2cccc3c2-c2c1ccc1c4c(-c5cccc(-c6ccc(Cl)cc6)c5)cccc4n(c21)-c1ccccc1-3] | 70.1 |
| 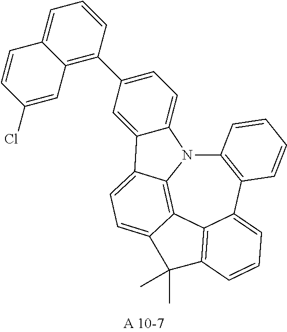 [SMILES: CC1(C)c2cccc3c2-c2c1ccc1c4cc(-c5cccc6ccc(Cl)cc56)ccc4n(c21)-c1ccccc1-3] | 69.1 |
| 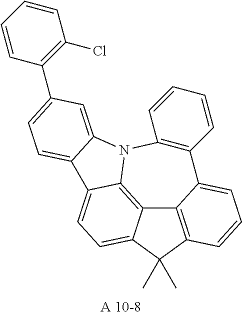 [SMILES: CC1(C)c2cccc3c2-c2c1ccc1c4ccc(-c5ccccc5Cl)cc4n(c21)-c1ccccc1-3] | 66.3 |
| 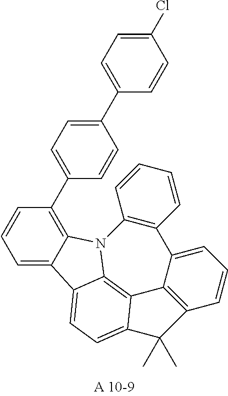 [SMILES: CC1(C)c2cccc3c2-c2c1ccc1c4cccc(-c5ccc(-c6ccc(Cl)cc6)cc5)c4n(c21)-c1ccccc1-3] | 65.5 |
| 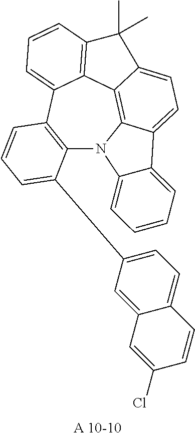 [SMILES: CC1(C)c2cccc3c2-c2c1ccc1c4ccccc4n(c21)-c1c(-c2ccc4ccc(Cl)cc4c2)cccc1-3] | 62.2 |
| 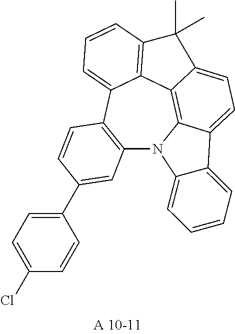 [SMILES: CC1(C)c2cccc3c2-c2c1ccc1c4ccccc4n(c21)-c1cc(-c2ccc(Cl)cc2)ccc1-3] | 72.1 |
| 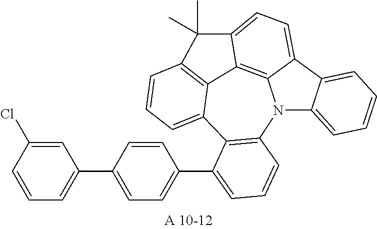 [SMILES: CC1(C)c2cccc3c2-c2c1ccc1c4ccccc4n(c21)-c1cccc(-c2ccc(-c4cccc(Cl)c4)cc2)c1-3] | 66.6 |
| 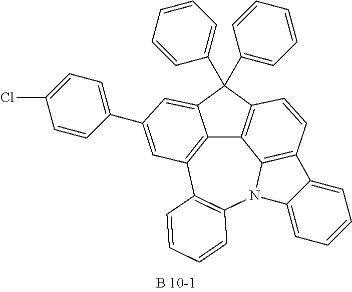 [SMILES: Clc1ccc(-c2cc3c4c(c2)C(c2ccccc2)(c2ccccc2)c2ccc5c6ccccc6n(c5c2-4)-c2ccccc2-3)cc1] | 68.2 |
| 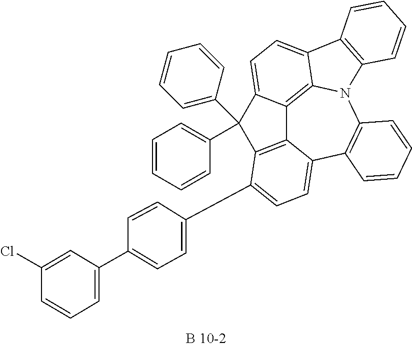 [SMILES: Clc1cccc(-c2ccc(-c3ccc4c5c3C(c3ccccc3)(c3ccccc3)c3ccc6c7ccccc7n(c6c3-5)-c3ccccc3-4)cc2)c1] | 60.1 |
| 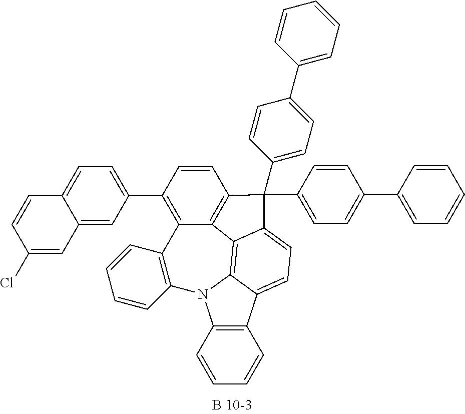 [SMILES: Clc1ccc2ccc(-c3ccc4c5c3-c3ccccc3-n3c6ccccc6c6ccc(c-5c63)C4(c3ccc(-c4ccccc4)cc3)c3ccc(-c4ccccc4)cc3)cc2c1] | 63.3 |
| 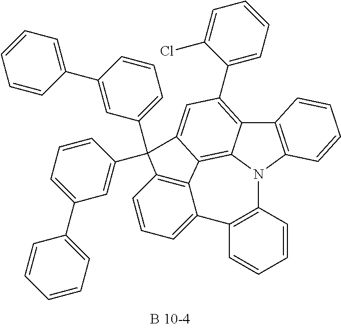 [SMILES: Clc1ccccc1-c1cc2c3c4c1c1ccccc1n4-c1ccccc1-c1cccc(c1-3)C2(c1cccc(-c2ccccc2)c1)c1cccc(-c2ccccc2)c1] | 63.2 |
| 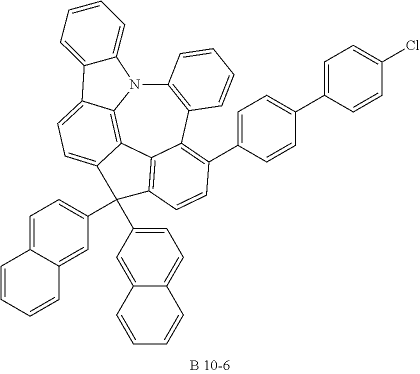 [SMILES: Clc1ccc(-c2ccc(-c3ccc4c5c3-c3ccccc3-n3c6ccccc6c6ccc(c-5c63)C4(c3ccc4ccccc4c3)c3ccc4ccccc4c3)cc2)cc1] | 65.6 |
| 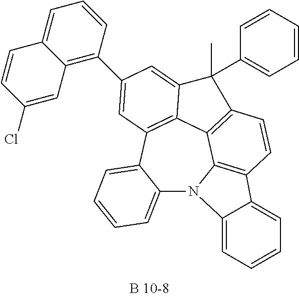 [SMILES: CC1(c2ccccc2)c2cc(-c3cccc4ccc(Cl)cc34)cc3c2-c2c1ccc1c4ccccc4n(c21)-c1ccccc1-3] | 63.4 |
| 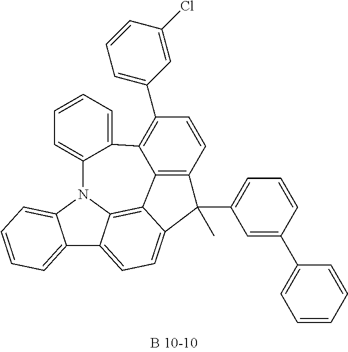 [SMILES: CC1(c2cccc(-c3ccccc3)c2)c2ccc(-c3cccc(Cl)c3)c3c2-c2c1ccc1c4ccccc4n(c21)-c1ccccc1-3] | 64.3 |
| 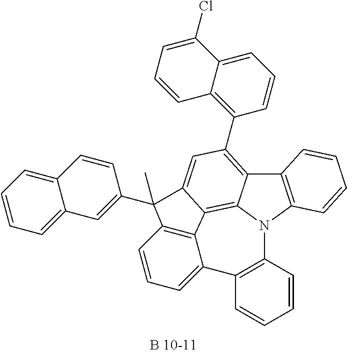 [SMILES: CC1(c2ccc3ccccc3c2)c2cccc3c2-c2c1cc(-c1cccc4c(Cl)cccc14)c1c4ccccc4n(c21)-c1ccccc1-3] | 67.5 |
| 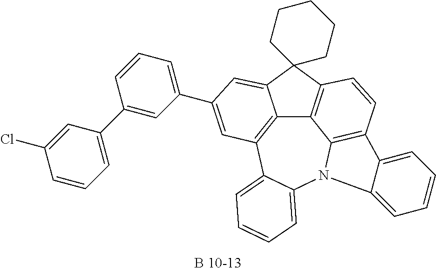 [SMILES: Clc1cccc(-c2cccc(-c3cc4c5c(c3)C3(CCCCC3)c3ccc6c7ccccc7n(c6c3-5)-c3ccccc3-4)c2)c1] | 68.1 |
| 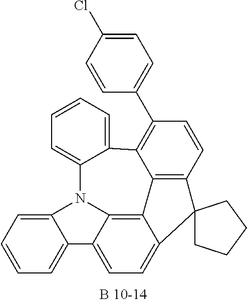 [SMILES: Clc1ccc(-c2ccc3c4c2-c2ccccc2-n2c5ccccc5c5ccc(c-4c52)C32CCCC2)cc1] | 61.6 |
| 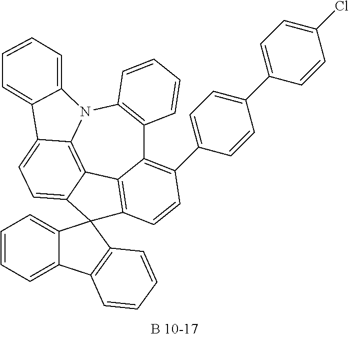 [SMILES: Clc1ccc(-c2ccc(-c3ccc4c5c3-c3ccccc3-n3c6ccccc6c6ccc(c-5c63)C43c4ccccc4-c4ccccc43)cc2)cc1] | 63.2 |
| 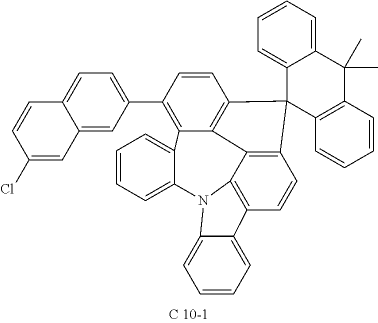 [SMILES: CC1(C)c2ccccc2C2(c3ccccc31)c1ccc(-c3ccc4ccc(Cl)cc4c3)c3c1-c1c2ccc2c4ccccc4n(c12)-c1ccccc1-3] | 62.5 |

### 9. Synthesis of Intermediates Y 12-X and Y 13-X

### Synthesis of an Intermediate A 12-1

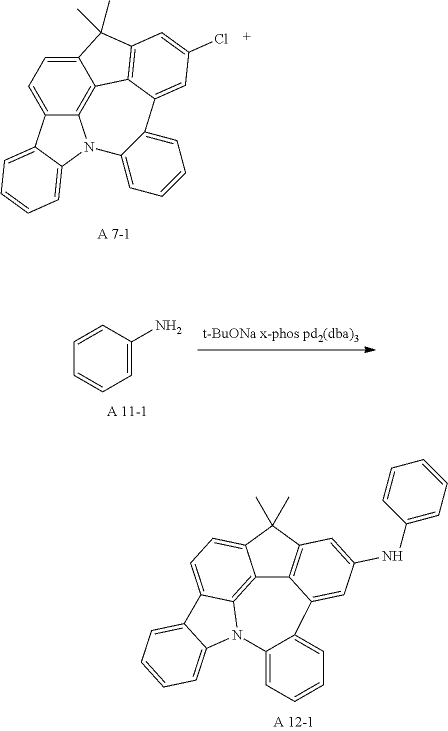 [SMILES: CC1(C)c2cc(Cl)cc3c2-c2c1ccc1c4ccccc4n(c21)-c1ccccc1-3 ; Nc1ccccc1 ; CC1(C)c2cc(Nc3ccccc3)cc3c2-c2c1ccc1c4ccccc4n(c21)-c1ccccc1-3]

Nitrogen was introduced into a three-necked flask equipped with a mechanical stirrer, a thermometer, and a spherical condenser at 0.100 L/min to allow nitrogen replacement for 15 min, then the intermediate A 7-1 (12.5 g, 31.90 mmol), a reactant A 11-1 (2.97 g, 31.90 mmol), tris(dibenzylideneacetone)dipalladium (0.29 g, 0.32 mmol), x-phos (0.30 g, 0.64 mmol), sodium tert-butoxide (4.60 g, 47.85 mmol), and toluene (330 mL) were added, and a resulting mixture was heated to 105° C. to 110° C. and stirred for 1 h. After the reaction was completed, a resulting reaction mixture was cooled to room temperature; toluene was added for extraction, the combined organic phases were washed with water, a resulting organic phase was dried with anhydrous magnesium sulfate, and filtered, and a filtrate was concentrated in vacuum to obtain a crude product; and the crude product was purified by recrystallization with a mixture of DCM and n-heptane to obtain a solid intermediate A 12-1 (10.02 g, yield: 70.1%).

The intermediates Y 12-X and Y 13-X (each X was an integer of 1 to 18 and each Y was A, B, or C) shown in table 17 below were each synthesized with reference to the synthesis method of the intermediate A 12-1, wherein the intermediates Y Z-X (each X was an integer of 1 to 18, each Y was A, B, or C, and each Z was 7 or 10) were used instead of the intermediate A 7-1 and the reactants A 11-X (each X was an integer of 1 to 13) were used instead of the reactant A 11-1.

TABLE 17

| Intermediate Y Z-X | Reactant A 11-X |
|---|---|
| 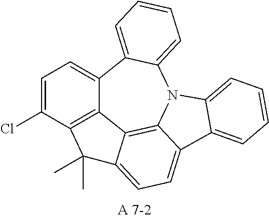 [SMILES: CC1(C)c2ccc3c4ccccc4n4c3c2-c2c(ccc(Cl)c21)-c1ccccc1-4] | 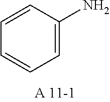 |
| 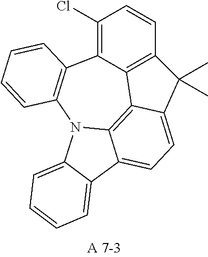 [SMILES: CC1(C)c2ccc(Cl)c3c2-c2c1ccc1c4ccccc4n(c21)-c1ccccc1-3] | 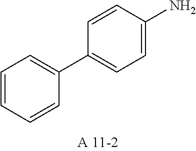 [SMILES: Nc1ccc(-c2ccccc2)cc1] |
| 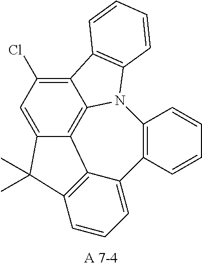 [SMILES: CC1(C)c2cccc3c2-c2c1cc(Cl)c1c4ccccc4n(c21)-c1ccccc1-3] | 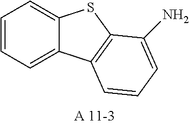 [SMILES: Nc1cccc2c1sc1ccccc12] |
| 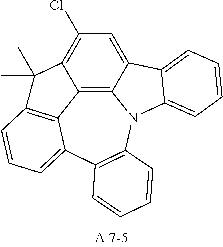 [SMILES: CC1(C)c2cccc3c2-c2c1c(Cl)cc1c4ccccc4n(c21)-c1ccccc1-3] | 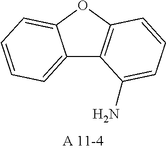 [SMILES: Nc1cccc2oc3ccccc3c12] |
| 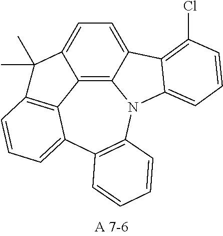 [SMILES: CC1(C)c2cccc3c2-c2c1ccc1c4c(Cl)cccc4n(c21)-c1ccccc1-3] | 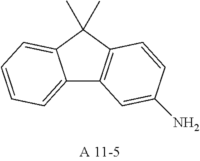 [SMILES: CC1(C)c2ccccc2-c2cc(N)ccc21] |
| 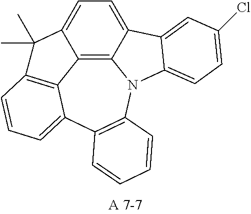 [SMILES: CC1(C)c2cccc3c2-c2c1ccc1c4cc(Cl)ccc4n(c21)-c1ccccc1-3] | 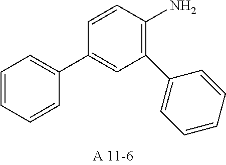 [SMILES: Nc1ccc(-c2ccccc2)cc1-c1ccccc1] |
| 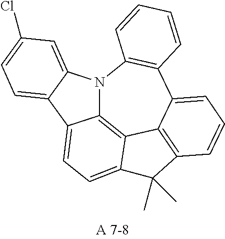 [SMILES: CC1(C)c2cccc3c2-c2c1ccc1c4ccc(Cl)cc4n(c21)-c1ccccc1-3] | 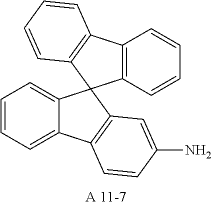 [SMILES: Nc1ccc2c(c1)C1(c3ccccc3-c3ccccc31)c1ccccc1-2] |
| 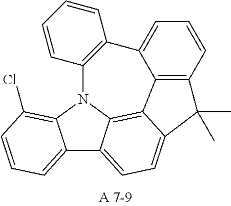 [SMILES: CC1(C)c2cccc3c2-c2c1ccc1c4cccc(Cl)c4n(c21)-c1ccccc1-3] | 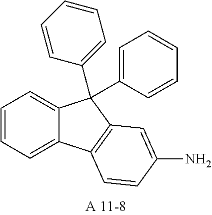 [SMILES: Nc1ccc2c(c1)C(c1ccccc1)(c1ccccc1)c1ccccc1-2] |
| 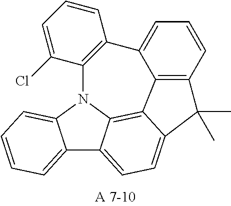 [SMILES: CC1(C)c2cccc3c2-c2c1ccc1c4ccccc4n(c21)-c1c(Cl)cccc1-3] | 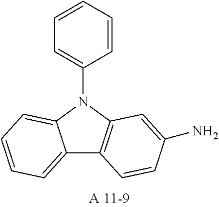 [SMILES: Nc1ccc2c3ccccc3n(-c3ccccc3)c2c1] |
| 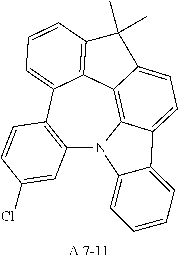 [SMILES: CC1(C)c2cccc3c2-c2c1ccc1c4ccccc4n(c21)-c1cc(Cl)ccc1-3] | 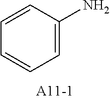 |
| 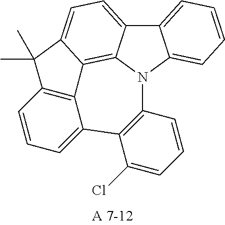 [SMILES: CC1(C)c2cccc3c2-c2c1ccc1c4ccccc4n(c21)-c1cccc(Cl)c1-3] | 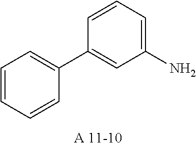 [SMILES: Nc1cccc(-c2ccccc2)c1] |
| 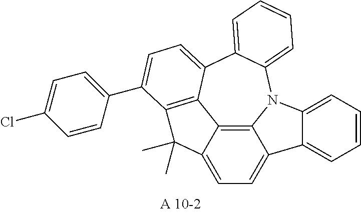 [SMILES: CC1(C)c2ccc3c4ccccc4n4c3c2-c2c(ccc(-c3ccc(Cl)cc3)c21)-c1ccccc1-4] | 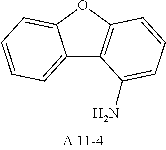 [SMILES: Nc1cccc2oc3ccccc3c12] |
| 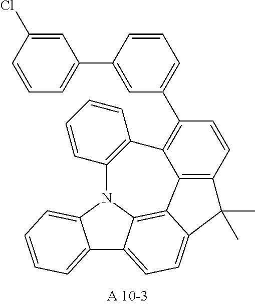 [SMILES: CC1(C)c2ccc(-c3cccc(-c4cccc(Cl)c4)c3)c3c2-c2c1ccc1c4ccccc4n(c21)-c1ccccc1-3] | 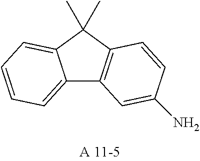 [SMILES: CC1(C)c2ccccc2-c2cc(N)ccc21] |
| 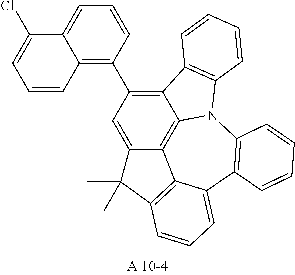 [SMILES: CC1(C)c2cccc3c2-c2c1cc(-c1cccc4c(Cl)cccc14)c1c4ccccc4n(c21)-c1ccccc1-3] | 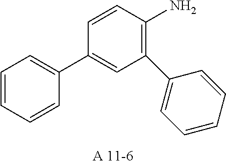 [SMILES: Nc1ccc(-c2ccccc2)cc1-c1ccccc1] |
| 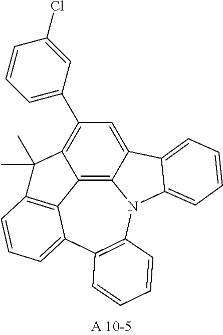 [SMILES: CC1(C)c2cccc3c2-c2c1c(-c1cccc(Cl)c1)cc1c4ccccc4n(c21)-c1ccccc1-3] | 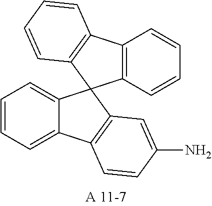 [SMILES: Nc1ccc2c(c1)C1(c3ccccc3-c3ccccc31)c1ccccc1-2] |
| 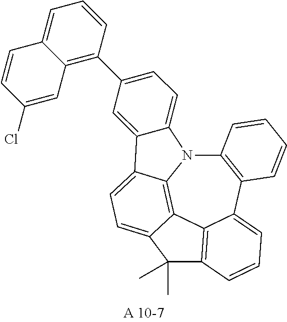 [SMILES: CC1(C)c2cccc3c2-c2c1ccc1c4cc(-c5cccc6ccc(Cl)cc56)ccc4n(c21)-c1ccccc1-3] | 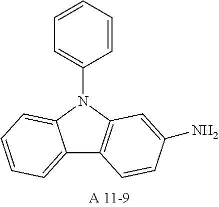 [SMILES: Nc1ccc2c3ccccc3n(-c3ccccc3)c2c1] |
| 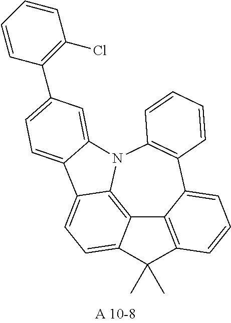 [SMILES: CC1(C)c2cccc3c2-c2c1ccc1c4ccc(-c5ccccc5Cl)cc4n(c21)-c1ccccc1-3] | 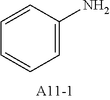 |
| 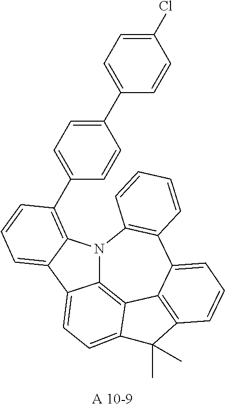 [SMILES: CC1(C)c2cccc3c2-c2c1ccc1c4cccc(-c5ccc(-c6ccc(Cl)cc6)cc5)c4n(c21)-c1ccccc1-3] | 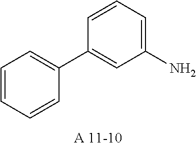 [SMILES: Nc1cccc(-c2ccccc2)c1] |
| 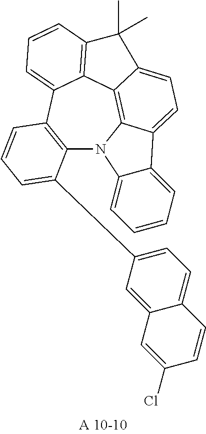 [SMILES: CC1(C)c2cccc3c2-c2c1ccc1c4ccccc4n(c21)-c1c(-c2ccc4ccc(Cl)cc4c2)cccc1-3] | 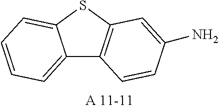 [SMILES: Nc1ccc2c(c1)sc1ccccc12] |
| 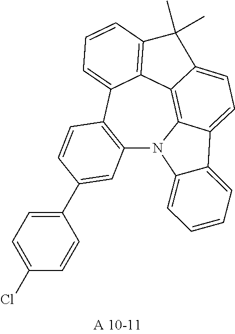 [SMILES: CC1(C)c2cccc3c2-c2c1ccc1c4ccccc4n(c21)-c1cc(-c2ccc(Cl)cc2)ccc1-3] | 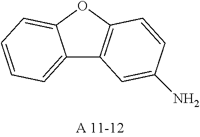 [SMILES: Nc1ccc2oc3ccccc3c2c1] |
| 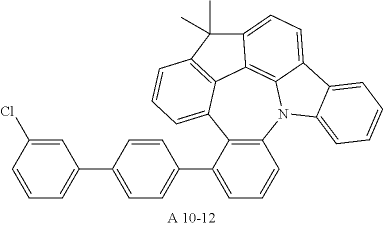 [SMILES: CC1(C)c2cccc3c2-c2c1ccc1c4ccccc4n(c21)-c1cccc(-c2ccc(-c4cccc(Cl)c4)cc2)c1-3] | 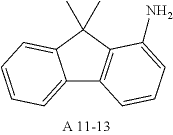 [SMILES: CC1(C)c2ccccc2-c2cccc(N)c21] |
| 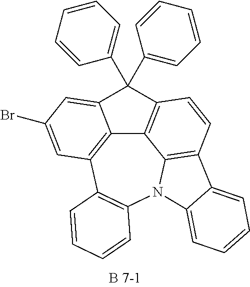 [SMILES: Brc1cc2c3c(c1)C(c1ccccc1)(c1ccccc1)c1ccc4c5ccccc5n(c4c1-3)-c1ccccc1-2] | 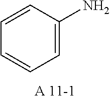 |
| 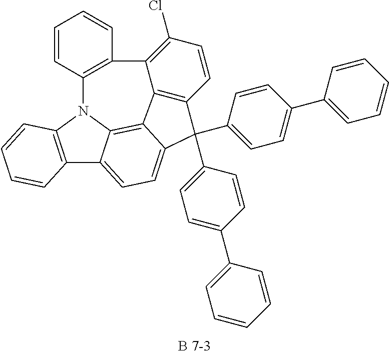 [SMILES: Clc1ccc2c3c1-c1ccccc1-n1c4ccccc4c4ccc(c-3c41)C2(c1ccc(-c2ccccc2)cc1)c1ccc(-c2ccccc2)cc1] | 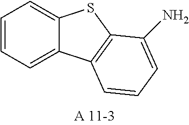 [SMILES: Nc1cccc2c1sc1ccccc12] |
| 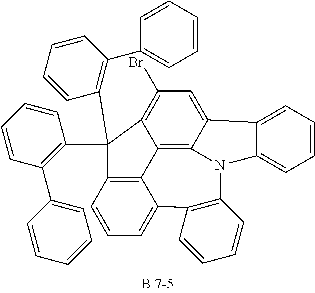 [SMILES: Brc1cc2c3ccccc3n3c2c2c1C(c1ccccc1-c1ccccc1)(c1ccccc1-c1ccccc1)c1cccc(c1-2)-c1ccccc1-3] | 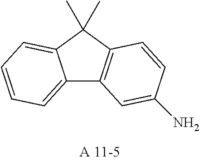 [SMILES: CC1(C)c2ccccc2-c2cc(N)ccc21] |
| 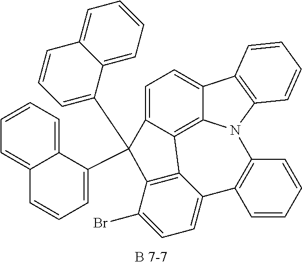 [SMILES: Brc1ccc2c3c1C(c1cccc4ccccc14)(c1cccc4ccccc14)c1ccc4c5ccccc5n(c4c1-3)-c1ccccc1-2] | 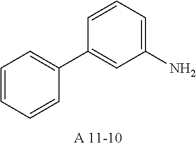 [SMILES: Nc1cccc(-c2ccccc2)c1] |
| 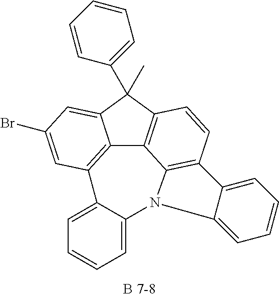 [SMILES: CC1(c2ccccc2)c2cc(Br)cc3c2-c2c1ccc1c4ccccc4n(c21)-c1ccccc1-3] | 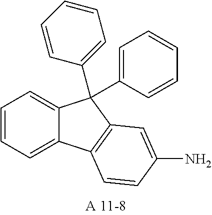 [SMILES: Nc1ccc2c(c1)C(c1ccccc1)(c1ccccc1)c1ccccc1-2] |
| 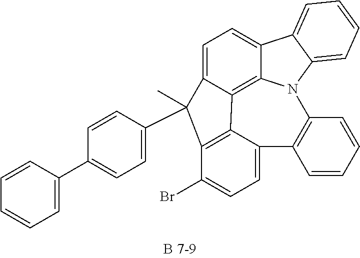 [SMILES: CC1(c2ccc(-c3ccccc3)cc2)c2ccc3c4ccccc4n4c3c2-c2c(ccc(Br)c21)-c1ccccc1-4] | 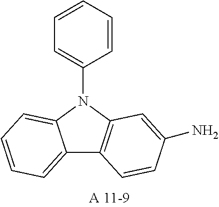 [SMILES: Nc1ccc2c3ccccc3n(-c3ccccc3)c2c1] |
| 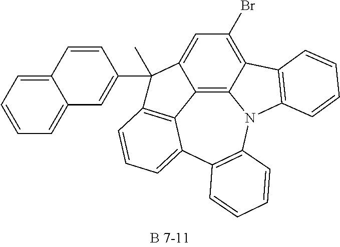 [SMILES: CC1(c2ccc3ccccc3c2)c2cccc3c2-c2c1cc(Br)c1c4ccccc4n(c21)-c1ccccc1-3] | 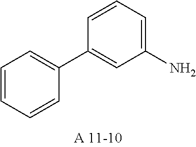 [SMILES: Nc1cccc(-c2ccccc2)c1] |
| 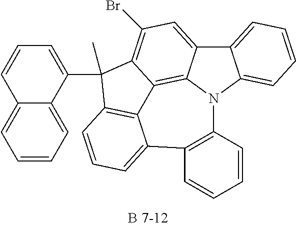 [SMILES: CC1(c2cccc3ccccc23)c2cccc3c2-c2c1c(Br)cc1c4ccccc4n(c21)-c1ccccc1-3] | 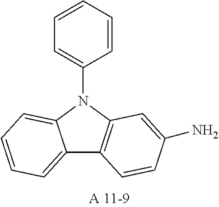 [SMILES: Nc1ccc2c3ccccc3n(-c3ccccc3)c2c1] |
| 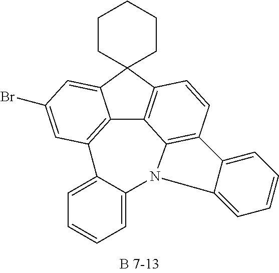 [SMILES: Brc1cc2c3c(c1)C1(CCCCC1)c1ccc4c5ccccc5n(c4c1-3)-c1ccccc1-2] | 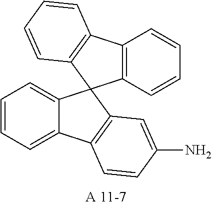 [SMILES: Nc1ccc2c(c1)C1(c3ccccc3-c3ccccc31)c1ccccc1-2] |
| 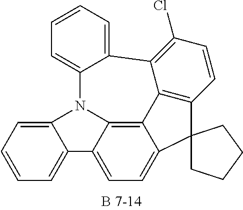 [SMILES: Clc1ccc2c3c1-c1ccccc1-n1c4ccccc4c4ccc(c-3c41)C21CCCC1] | 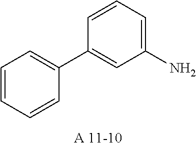 [SMILES: Nc1cccc(-c2ccccc2)c1] |
| 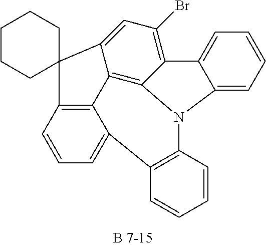 [SMILES: Brc1cc2c3c4c1c1ccccc1n4-c1ccccc1-c1cccc(c1-3)C21CCCCC1] | 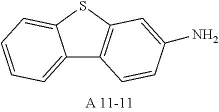 [SMILES: Nc1ccc2c(c1)sc1ccccc12] |
| 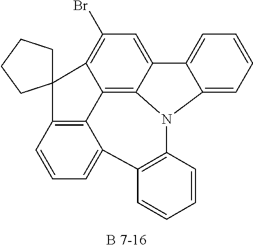 [SMILES: Brc1cc2c3ccccc3n3c2c2c1C1(CCCC1)c1cccc(c1-2)-c1ccccc1-3] | 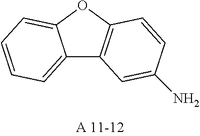 [SMILES: Nc1ccc2oc3ccccc3c2c1] |
| 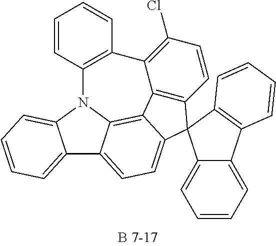 [SMILES: Clc1ccc2c3c1-c1ccccc1-n1c4ccccc4c4ccc(c-3c41)C21c2ccccc2-c2ccccc21] | 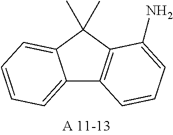 [SMILES: CC1(C)c2ccccc2-c2cccc(N)c21] |
| 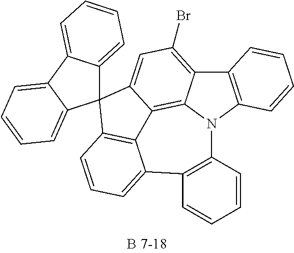 [SMILES: Brc1cc2c3c4c1c1ccccc1n4-c1ccccc1-c1cccc(c1-3)C21c2ccccc2-c2ccccc21] | 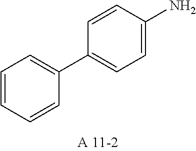 [SMILES: Nc1ccc(-c2ccccc2)cc1] |
| 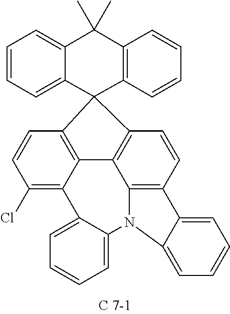 [SMILES: CC1(C)c2ccccc2C2(c3ccccc31)c1ccc(Cl)c3c1-c1c2ccc2c4ccccc4n(c12)-c1ccccc1-3] | 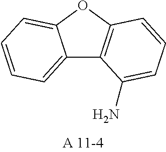 [SMILES: Nc1cccc2oc3ccccc3c12] |
| 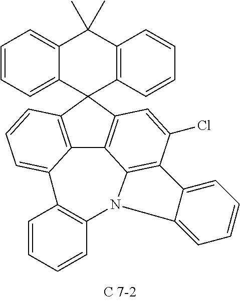 [SMILES: CC1(C)c2ccccc2C2(c3ccccc31)c1cccc3c1-c1c2cc(Cl)c2c4ccccc4n(c12)-c1ccccc1-3] | 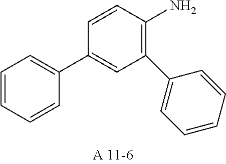 [SMILES: Nc1ccc(-c2ccccc2)cc1-c1ccccc1] |
| 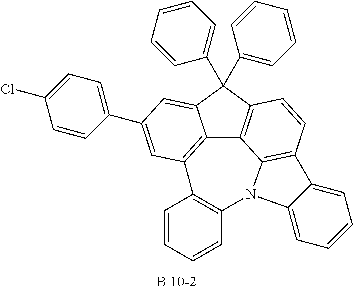 [SMILES: Clc1ccc(-c2cc3c4c(c2)C(c2ccccc2)(c2ccccc2)c2ccc5c6ccccc6n(c5c2-4)-c2ccccc2-3)cc1] | 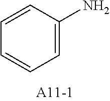 |
| 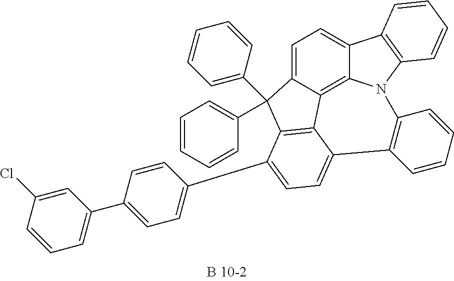 [SMILES: Clc1cccc(-c2ccc(-c3ccc4c5c3C(c3ccccc3)(c3ccccc3)c3ccc6c7ccccc7n(c6c3-5)-c3ccccc3-4)cc2)c1] | 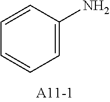 |
| 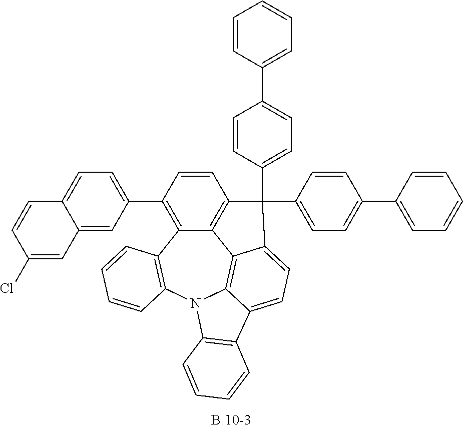 [SMILES: Clc1ccc2ccc(-c3ccc4c5c3-c3ccccc3-n3c6ccccc6c6ccc(c-5c63)C4(c3ccc(-c4ccccc4)cc3)c3ccc(-c4ccccc4)cc3)cc2c1] | 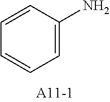 |
| 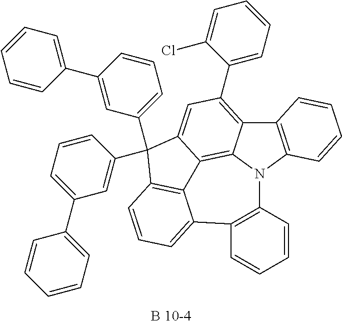 [SMILES: Clc1ccccc1-c1cc2c3c4c1c1ccccc1n4-c1ccccc1-c1cccc(c1-3)C2(c1cccc(-c2ccccc2)c1)c1cccc(-c2ccccc2)c1] | 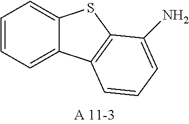 [SMILES: Nc1cccc2c1sc1ccccc12] |
| 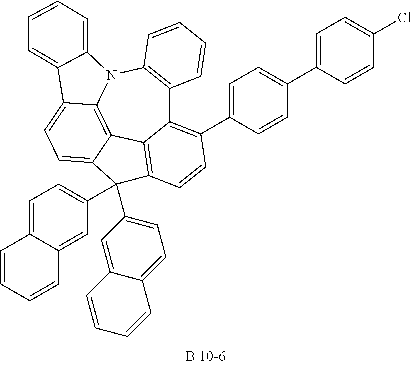 [SMILES: Clc1ccc(-c2ccc(-c3ccc4c5c3-c3ccccc3-n3c6ccccc6c6ccc(c-5c63)C4(c3ccc4ccccc4c3)c3ccc4ccccc4c3)cc2)cc1] | 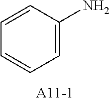 |
| 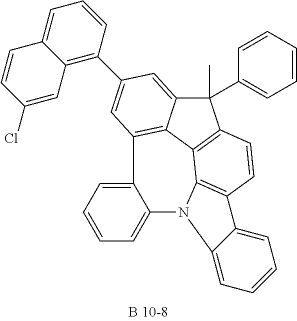 [SMILES: CC1(c2ccccc2)c2cc(-c3cccc4ccc(Cl)cc34)cc3c2-c2c1ccc1c4ccccc4n(c21)-c1ccccc1-3] | 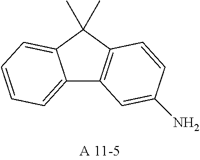 [SMILES: CC1(C)c2ccccc2-c2cc(N)ccc21] |
| 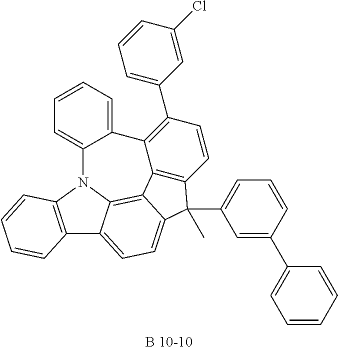 [SMILES: CC1(c2cccc(-c3ccccc3)c2)c2ccc(-c3cccc(Cl)c3)c3c2-c2c1ccc1c4ccccc4n(c21)-c1ccccc1-3] | 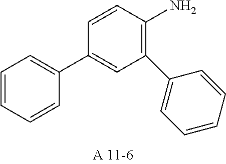 [SMILES: Nc1ccc(-c2ccccc2)cc1-c1ccccc1] |
| 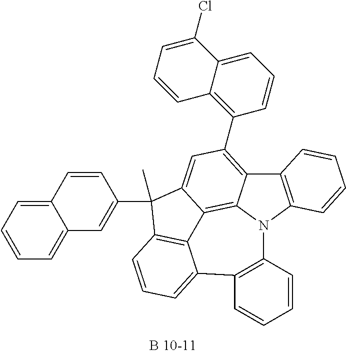 [SMILES: CC1(c2ccc3ccccc3c2)c2cccc3c2-c2c1cc(-c1cccc4c(Cl)cccc14)c1c4ccccc4n(c21)-c1ccccc1-3] | 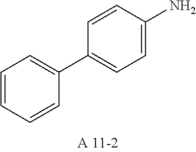 [SMILES: Nc1ccc(-c2ccccc2)cc1] |
| 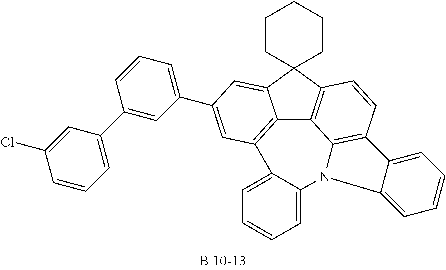 [SMILES: Clc1cccc(-c2cccc(-c3cc4c5c(c3)C3(CCCCC3)c3ccc6c7ccccc7n(c6c3-5)-c3ccccc3-4)c2)c1] | 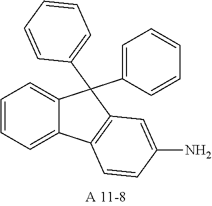 [SMILES: Nc1ccc2c(c1)C(c1ccccc1)(c1ccccc1)c1ccccc1-2] |
| 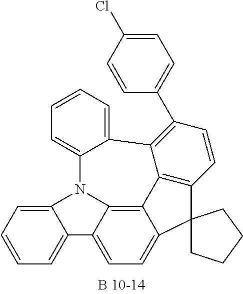 [SMILES: Clc1ccc(-c2ccc3c4c2-c2ccccc2-n2c5ccccc5c5ccc(c-4c52)C32CCCC2)cc1] | 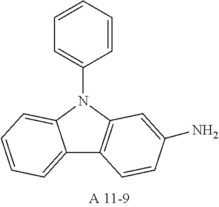 [SMILES: Nc1ccc2c3ccccc3n(-c3ccccc3)c2c1] |
| 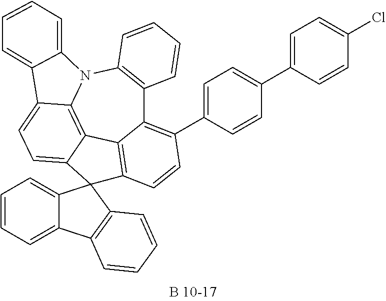 [SMILES: Clc1ccc(-c2ccc(-c3ccc4c5c3-c3ccccc3-n3c6ccccc6c6ccc(c-5c63)C43c4ccccc4-c4ccccc43)cc2)cc1] | 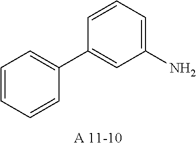 [SMILES: Nc1cccc(-c2ccccc2)c1] |
| 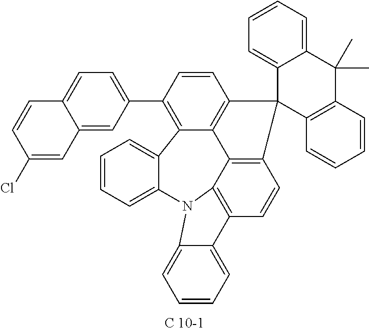 [SMILES: CC1(C)c2ccccc2C2(c3ccccc31)c1ccc(-c3ccc4ccc(Cl)cc4c3)c3c1-c1c2ccc2c4ccccc4n(c12)-c1ccccc1-3] | 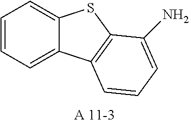 [SMILES: Nc1cccc2c1sc1ccccc12] |
| Y 12-X/Y 13-X | Yield (%) |
| 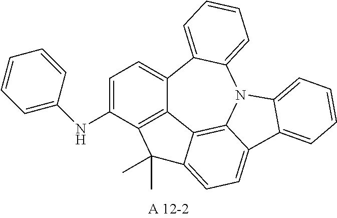 [SMILES: CC1(C)c2ccc3c4ccccc4n4c3c2-c2c(ccc(Nc3ccccc3)c21)-c1ccccc1-4] | 68.3 |
| 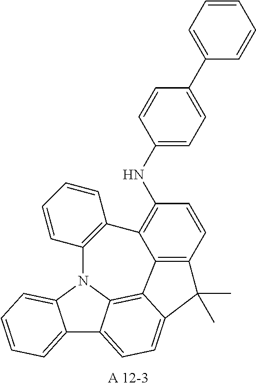 [SMILES: CC1(C)c2ccc(Nc3ccc(-c4ccccc4)cc3)c3c2-c2c1ccc1c4ccccc4n(c21)-c1ccccc1-3] | 67.1 |
| 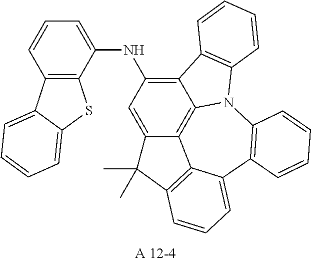 [SMILES: CC1(C)c2cccc3c2-c2c1cc(Nc1cccc4c1sc1ccccc14)c1c4ccccc4n(c21)-c1ccccc1-3] | 65.3 |
| 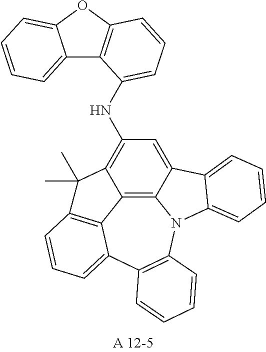 [SMILES: CC1(C)c2cccc3c2-c2c1c(Nc1cccc4oc5ccccc5c14)cc1c4ccccc4n(c21)-c1ccccc1-3] | 65.5 |
| 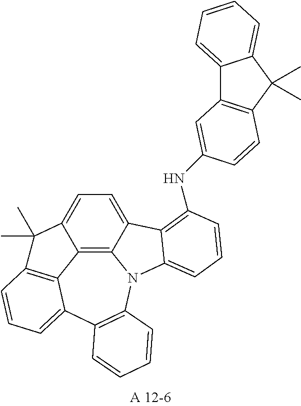 | 68.1 |
| 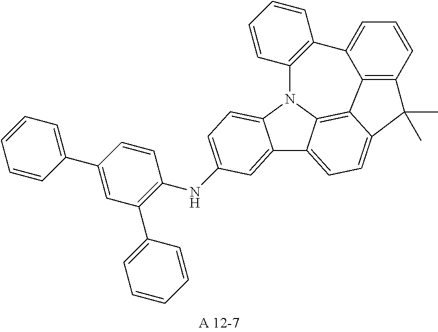 [SMILES: CC1(C)c2cccc3c2-c2c1ccc1c4cc(Nc5ccc(-c6ccccc6)cc5-c5ccccc5)ccc4n(c21)-c1ccccc1-3] | 66.3 |
| 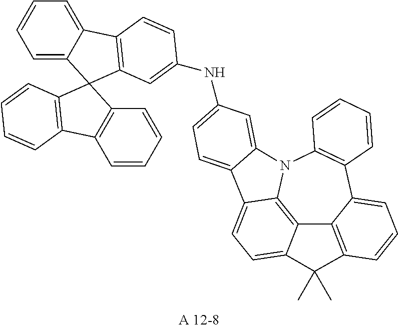 [SMILES: CC1(C)c2cccc3c2-c2c1ccc1c4ccc(Nc5ccc6c(c5)C5(c7ccccc7-c7ccccc75)c5ccccc5-6)cc4n(c21)-c1ccccc1-3] | 69.1 |
| 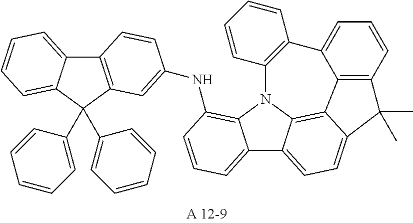 [SMILES: CC1(C)c2cccc3c2-c2c1ccc1c4cccc(Nc5ccc6c(c5)C(c5ccccc5)(c5ccccc5)c5ccccc5-6)c4n(c21)-c1ccccc1-3] | 60.8 |
| 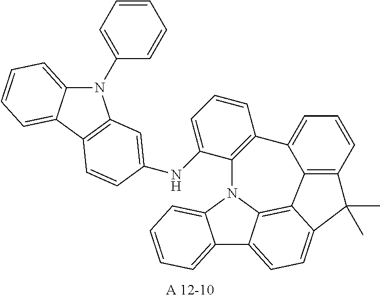 [SMILES: CC1(C)c2cccc3c2-c2c1ccc1c4ccccc4n(c21)-c1c(Nc2ccc4c5ccccc5n(-c5ccccc5)c4c2)cccc1-3] | 58.1 |
| 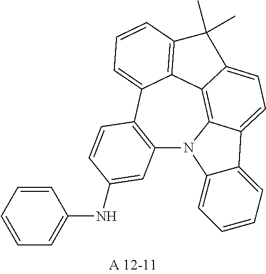 [SMILES: CC1(C)c2cccc3c2-c2c1ccc1c4ccccc4n(c21)-c1cc(Nc2ccccc2)ccc1-3] | 72.3 |
| 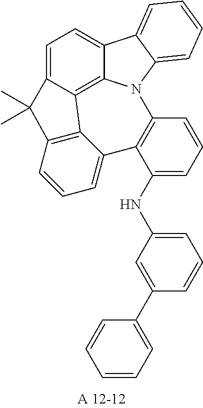 [SMILES: CC1(C)c2cccc3c2-c2c1ccc1c4ccccc4n(c21)-c1cccc(Nc2cccc(-c4ccccc4)c2)c1-3] | 62.2 |
| 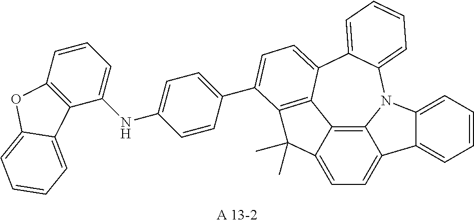 [SMILES: CC1(C)c2ccc3c4ccccc4n4c3c2-c2c(ccc(-c3ccc(Nc5cccc6oc7ccccc7c56)cc3)c21)-c1ccccc1-4] | 63.5 |
| 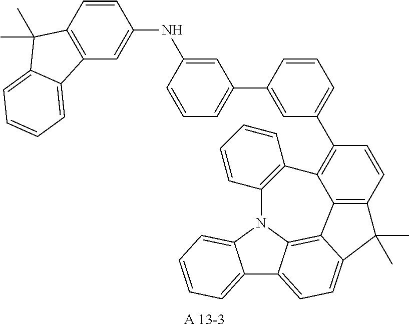 | 70.1 |
| 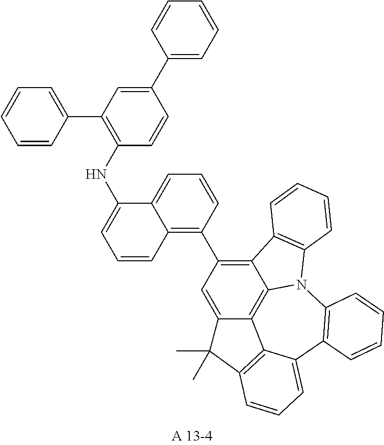 [SMILES: CC1(C)c2cccc3c2-c2c1cc(-c1cccc4c(Nc5ccc(-c6ccccc6)cc5-c5ccccc5)cccc14)c1c4ccccc4n(c21)-c1ccccc1-3] | 65.3 |
| 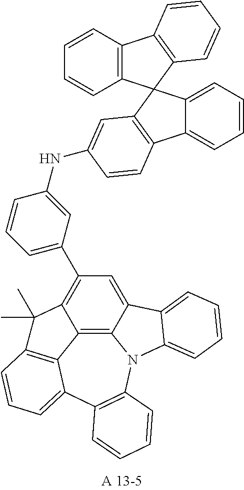 [SMILES: CC1(C)c2cccc3c2-c2c1c(-c1cccc(Nc4ccc5c(c4)C4(c6ccccc6-c6ccccc64)c4ccccc4-5)c1)cc1c4ccccc4n(c21)-c1ccccc1-3] | 66.0 |
| 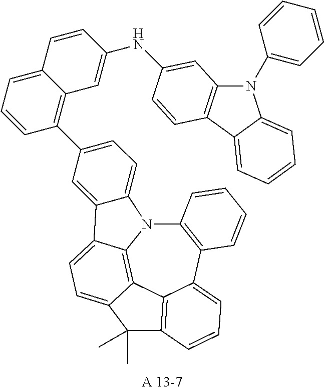 [SMILES: CC1(C)c2cccc3c2-c2c1ccc1c4cc(-c5cccc6ccc(Nc7ccc8c9ccccc9n(-c9ccccc9)c8c7)cc56)ccc4n(c21)-c1ccccc1-3] | 67.8 |
| 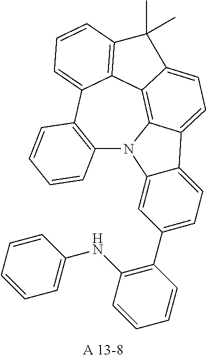 [SMILES: CC1(C)c2cccc3c2-c2c1ccc1c4ccc(-c5ccccc5Nc5ccccc5)cc4n(c21)-c1ccccc1-3] | 70.3 |
| 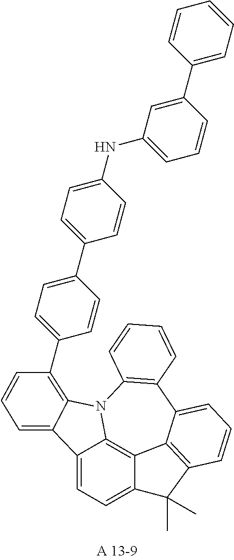 [SMILES: CC1(C)c2cccc3c2-c2c1ccc1c4cccc(-c5ccc(-c6ccc(Nc7cccc(-c8ccccc8)c7)cc6)cc5)c4n(c21)-c1ccccc1-3] | 69.1 |
| 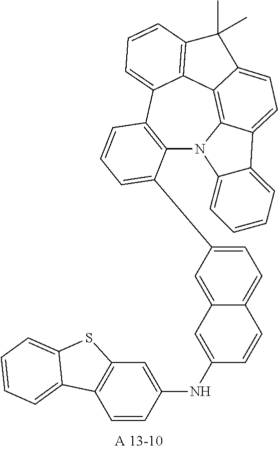 [SMILES: CC1(C)c2cccc3c2-c2c1ccc1c4ccccc4n(c21)-c1c(-c2ccc4ccc(Nc5ccc6c(c5)sc5ccccc56)cc4c2)cccc1-3] | 66.2 |
| 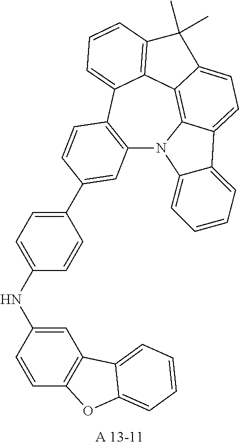 [SMILES: CC1(C)c2cccc3c2-c2c1ccc1c4ccccc4n(c21)-c1cc(-c2ccc(Nc4ccc5oc6ccccc6c5c4)cc2)ccc1-3] | 68.1 |
| 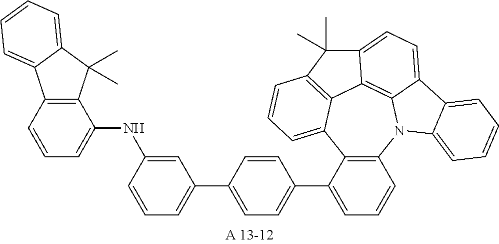 [SMILES: CC1(C)c2cccc3c2-c2c1ccc1c4ccccc4n(c21)-c1cccc(-c2ccc(-c4cccc(Nc5cccc6c5C(C)(C)c5ccccc5-6)c4)cc2)c1-3] | 63.5 |
| 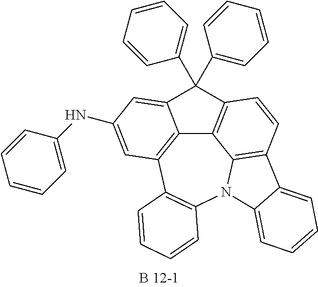 [SMILES: c1ccc(Nc2cc3c4c(c2)C(c2ccccc2)(c2ccccc2)c2ccc5c6ccccc6n(c5c2-4)-c2ccccc2-3)cc1] | 67.4 |
| 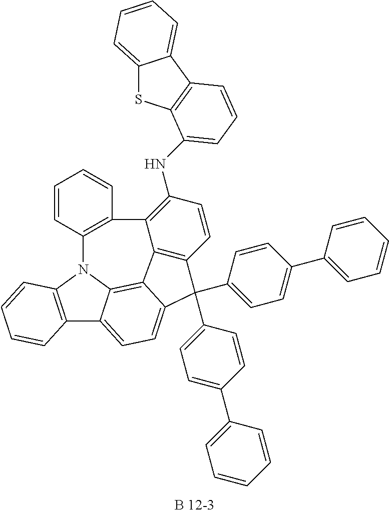 [SMILES: c1ccc(-c2ccc(C3(c4ccc(-c5ccccc5)cc4)c4ccc(Nc5cccc6c5sc5ccccc56)c5c4-c4c3ccc3c6ccccc6n(c43)-c3ccccc3-5)cc2)cc1] | 62.1 |
| 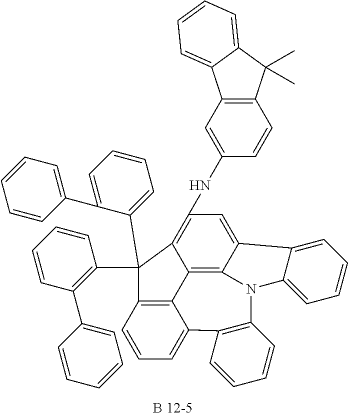 [SMILES: CC1(C)c2ccccc2-c2cc(Nc3cc4c5ccccc5n5c4c4c3C(c3ccccc3-c3ccccc3)(c3ccccc3-c3ccccc3)c3cccc(c3-4)-c3ccccc3-5)ccc21] | 56.3 |
| 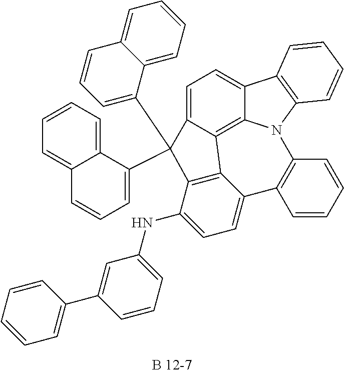 [SMILES: c1ccc(-c2cccc(Nc3ccc4c5c3C(c3cccc6ccccc36)(c3cccc6ccccc36)c3ccc6c7ccccc7n(c6c3-5)-c3ccccc3-4)c2)cc1] | 53.8 |
| 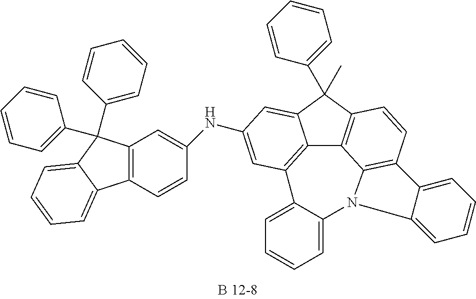 [SMILES: CC1(c2ccccc2)c2cc(Nc3ccc4c(c3)C(c3ccccc3)(c3ccccc3)c3ccccc3-4)cc3c2-c2c1ccc1c4ccccc4n(c21)-c1ccccc1-3] | 63.1 |
| 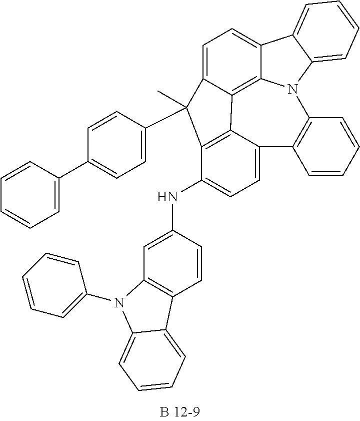 [SMILES: CC1(c2ccc(-c3ccccc3)cc2)c2ccc3c4ccccc4n4c3c2-c2c(ccc(Nc3ccc5c6ccccc6n(-c6ccccc6)c5c3)c21)-c1ccccc1-4] | 60.5 |
| 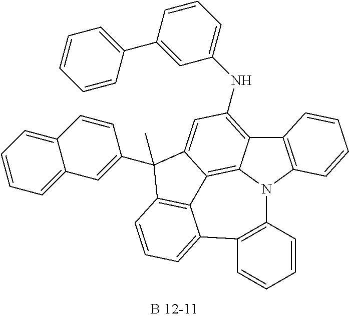 [SMILES: CC1(c2ccc3ccccc3c2)c2cccc3c2-c2c1cc(Nc1cccc(-c4ccccc4)c1)c1c4ccccc4n(c21)-c1ccccc1-3] | 65.2 |
| 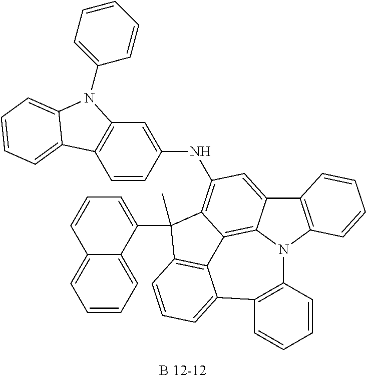 [SMILES: CC1(c2cccc3ccccc23)c2cccc3c2-c2c1c(Nc1ccc4c5ccccc5n(-c5ccccc5)c4c1)cc1c4ccccc4n(c21)-c1ccccc1-3] | 61.6 |
| 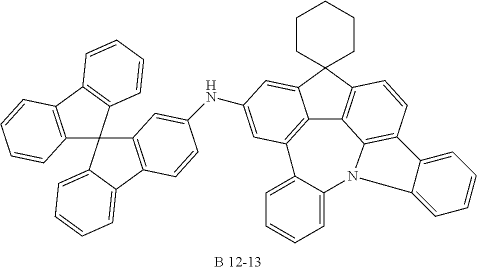 [SMILES: c1ccc2c(c1)-c1cc(Nc3ccc4c(c3)C3(c5ccccc5-c5ccccc53)c3ccccc3-4)cc3c1-c1c(ccc4c5ccccc5n-2c14)C31CCCCC1] | 64.1 |
| 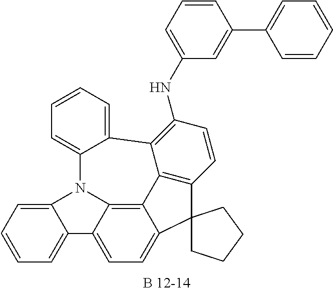 [SMILES: c1ccc(-c2cccc(Nc3ccc4c5c3-c3ccccc3-n3c6ccccc6c6ccc(c-5c63)C43CCCC3)c2)cc1] | 59.8 |
| 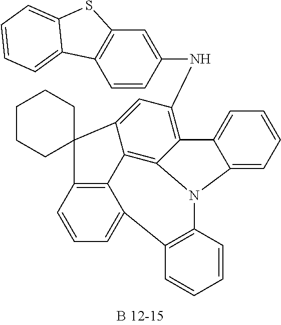 [SMILES: c1ccc2c(c1)-c1cccc3c1-c1c(cc(Nc4ccc5c(c4)sc4ccccc45)c4c5ccccc5n-2c14)C31CCCCC1] | 61.5 |
| 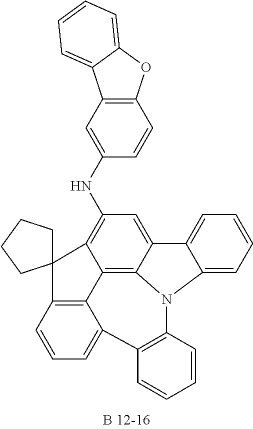 [SMILES: c1ccc2c(c1)-c1cccc3c1-c1c(c(Nc4ccc5oc6ccccc6c5c4)cc4c5ccccc5n-2c14)C31CCCC1] | 66.1 |
| 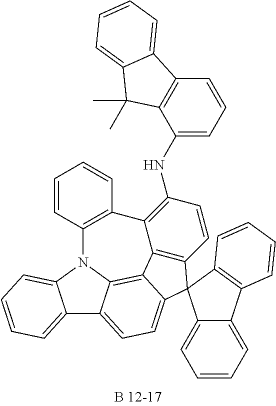 [SMILES: CC1(C)c2ccccc2-c2cccc(Nc3ccc4c5c3-c3ccccc3-n3c6ccccc6c6ccc(c-5c63)C43c4ccccc4-c4ccccc43)c21] | 63.2 |
| 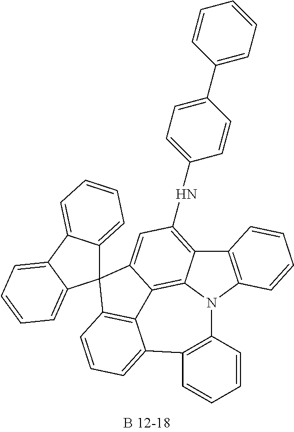 [SMILES: c1ccc(-c2ccc(Nc3cc4c5c6c3c3ccccc3n6-c3ccccc3-c3cccc(c3-5)C43c4ccccc4-c4ccccc43)cc2)cc1] | 66.3 |
| 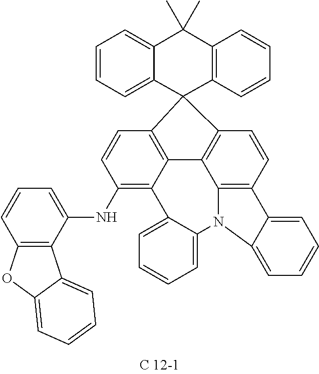 [SMILES: CC1(C)c2ccccc2C2(c3ccccc31)c1ccc(Nc3cccc4oc5ccccc5c34)c3c1-c1c2ccc2c4ccccc4n(c12)-c1ccccc1-3] | 57.5 |
| 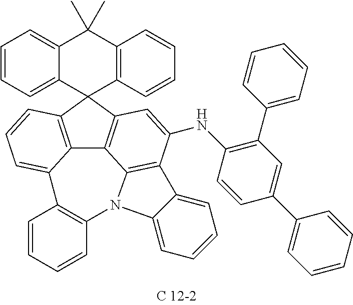 [SMILES: CC1(C)c2ccccc2C2(c3ccccc31)c1cccc3c1-c1c2cc(Nc2ccc(-c4ccccc4)cc2-c2ccccc2)c2c4ccccc4n(c12)-c1ccccc1-3] | 61.1 |
| 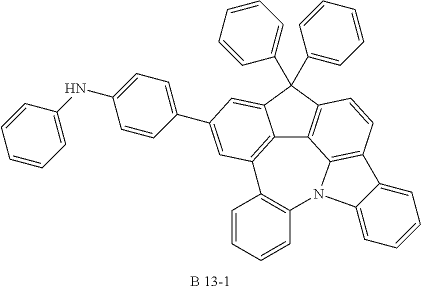 [SMILES: c1ccc(Nc2ccc(-c3cc4c5c(c3)C(c3ccccc3)(c3ccccc3)c3ccc6c7ccccc7n(c6c3-5)-c3ccccc3-4)cc2)cc1] | 65.3 |
| 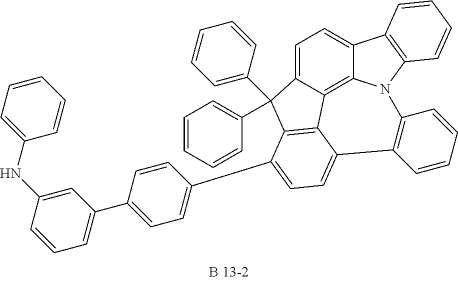 [SMILES: c1ccc(Nc2cccc(-c3ccc(-c4ccc5c6c4C(c4ccccc4)(c4ccccc4)c4ccc7c8ccccc8n(c7c4-6)-c4ccccc4-5)cc3)c2)cc1] | 67.8 |
| 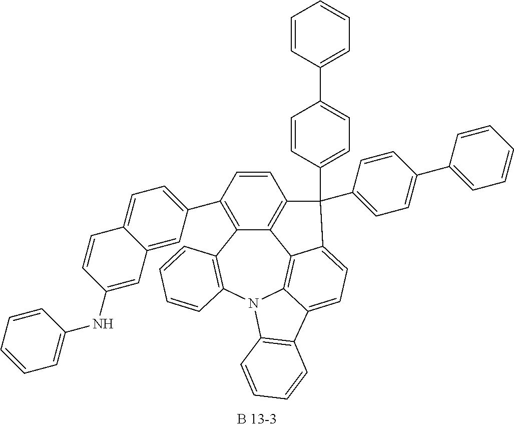 [SMILES: c1ccc(Nc2ccc3ccc(-c4ccc5c6c4-c4ccccc4-n4c7ccccc7c7ccc(c-6c74)C5(c4ccc(-c5ccccc5)cc4)c4ccc(-c5ccccc5)cc4)cc3c2)cc1] | 66.1 |
| 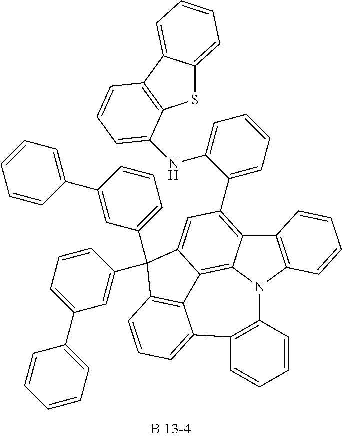 [SMILES: c1ccc(-c2cccc(C3(c4cccc(-c5ccccc5)c4)c4cccc5c4-c4c3cc(-c3ccccc3Nc3cccc6c3sc3ccccc36)c3c6ccccc6n(c43)-c3ccccc3-5)c2)cc1] | 60.5 |
| 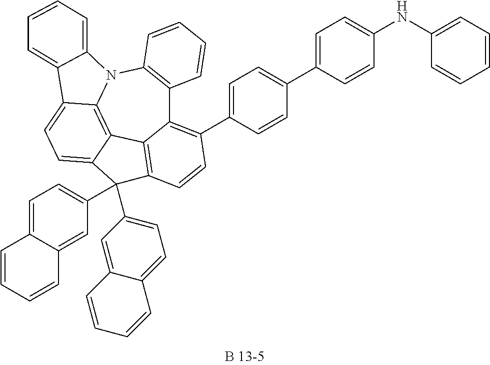 [SMILES: c1ccc(Nc2ccc(-c3ccc(-c4ccc5c6c4-c4ccccc4-n4c7ccccc7c7ccc(c-6c74)C5(c4ccc5ccccc5c4)c4ccc5ccccc5c4)cc3)cc2)cc1] | 56.3 |
| 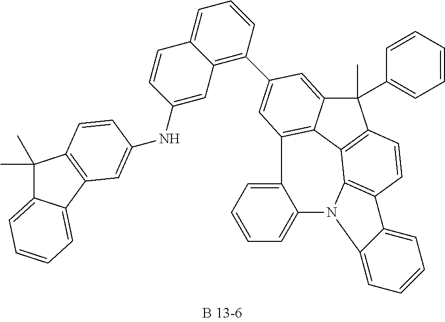 [SMILES: CC1(C)c2ccccc2-c2cc(Nc3ccc4cccc(-c5cc6c7c(c5)C(C)(c5ccccc5)c5ccc8c9ccccc9n(c8c5-7)-c5ccccc5-6)c4c3)ccc21] | 64.6 |
| 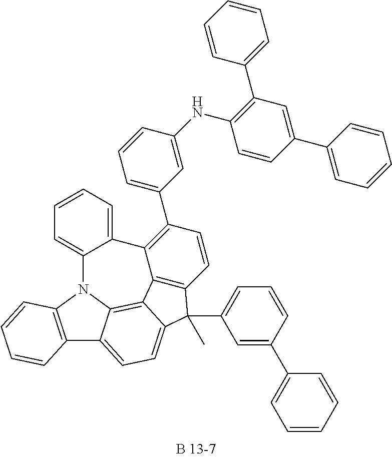 [SMILES: CC1(c2cccc(-c3ccccc3)c2)c2ccc(-c3cccc(Nc4ccc(-c5ccccc5)cc4-c4ccccc4)c3)c3c2-c2c1ccc1c4ccccc4n(c21)-c1ccccc1-3] | 67.5 |
| 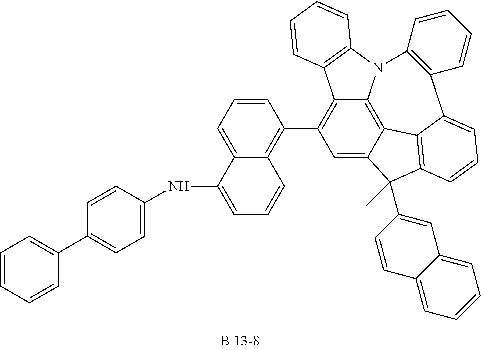 [SMILES: CC1(c2ccc3ccccc3c2)c2cccc3c2-c2c1cc(-c1cccc4c(Nc5ccc(-c6ccccc6)cc5)cccc14)c1c4ccccc4n(c21)-c1ccccc1-3] | 65.3 |
| 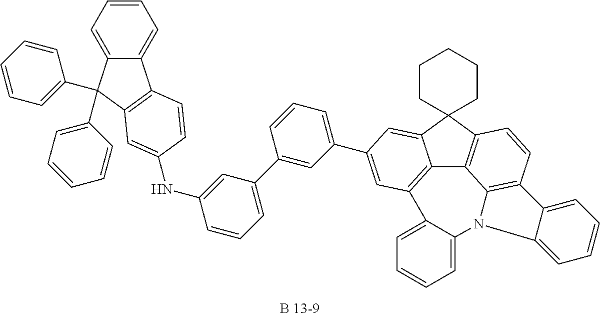 [SMILES: c1ccc(C2(c3ccccc3)c3ccccc3-c3ccc(Nc4cccc(-c5cccc(-c6cc7c8c(c6)C6(CCCCC6)c6ccc9c%10ccccc%10n(c9c6-8)-c6ccccc6-7)c5)c4)cc32)cc1] | 66.1 |
| 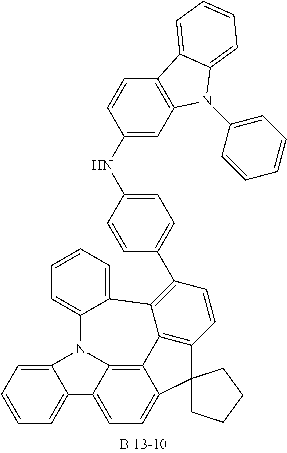 [SMILES: c1ccc(-n2c3ccccc3c3ccc(Nc4ccc(-c5ccc6c7c5-c5ccccc5-n5c8ccccc8c8ccc(c-7c85)C65CCCC5)cc4)cc32)cc1] | 60.6 |
| 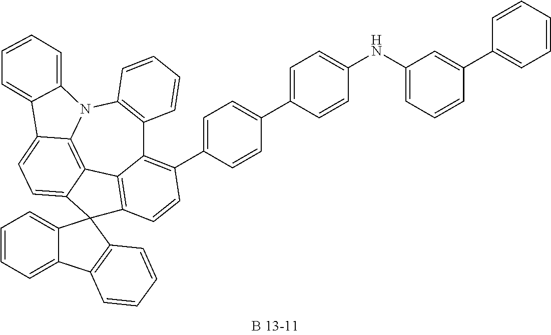 [SMILES: c1ccc(-c2cccc(Nc3ccc(-c4ccc(-c5ccc6c7c5-c5ccccc5-n5c8ccccc8c8ccc(c-7c85)C65c6ccccc6-c6ccccc65)cc4)cc3)c2)cc1] | 64.2 |
| 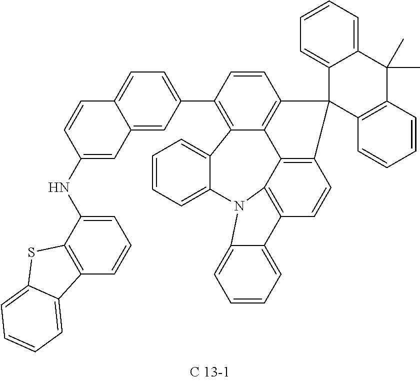 [SMILES: CC1(C)c2ccccc2C2(c3ccccc31)c1ccc(-c3ccc4ccc(Nc5cccc6c5sc5ccccc56)cc4c3)c3c1-c1c2ccc2c4ccccc4n(c12)-c1ccccc1-3] | 58.7 |

### 10. Synthesis of Compounds

### Synthesis of a Compound 1

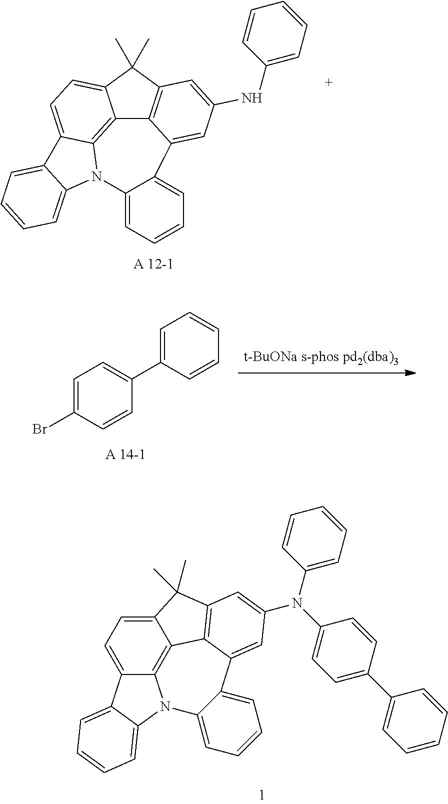 [SMILES: CC1(C)c2cc(Nc3ccccc3)cc3c2-c2c1ccc1c4ccccc4n(c21)-c1ccccc1-3 ; Brc1ccc(-c2ccccc2)cc1 ; CC1(C)c2cc(N(c3ccccc3)c3ccc(-c4ccccc4)cc3)cc3c2-c2c1ccc1c4ccccc4n(c21)-c1ccccc1-3]

Nitrogen was introduced into a three-necked flask equipped with a mechanical stirrer, a thermometer, and a spherical condenser at 0.100 L/min to allow nitrogen replacement for 15 min, then the intermediate A 12-1 (9.5 g, 21.18 mmol), a reactant A 14-1 (4.94 g, 21.18 mmol), tris(dibenzylideneacetone)dipalladium (0.19 g, 0.21 mmol), s-phos (0.174 g, 0.42 mmol), sodium tert-butoxide (3.05 g, 31.77 mmol), and toluene (76 mL) were added, and a resulting mixture was heated to 105° C. to 110° C. and stirred for 2 h. After the reaction was completed, a resulting reaction mixture was cooled to room temperature; toluene was added for extraction, the combined organic phase was washed with water, a resulting organic phase was dried with anhydrous magnesium sulfate, and filtered, and a filtrate was concentrated in vacuum to obtain a crude product; and the crude product was purified by recrystallization with a mixture of DCM and n-heptane to obtain a solid compound 1 (8.27 g, yield: 65.0%, MS: m/z=601.2[M+H]⁺).

The compounds shown in table 18 below were each synthesized with reference to the synthesis method of the compound 1, wherein the intermediates Y Z-X (each X was an integer of 1 to 18, each Y was A, B, or C, and each Z was 12 or 13) were used instead of the intermediate A 12-1 and the reactants A 14-X (each X was an integer of 1 to 16) were used instead of the reactant A 14-1.

TABLE 18

| Intermediate Y Z-X | Reactant A 14-X | |
|---|---|---|
| 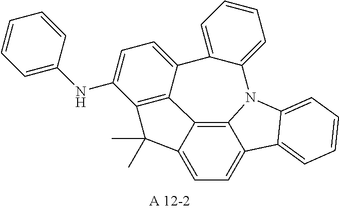 [SMILES: CC1(C)c2ccc3c4ccccc4n4c3c2-c2c(ccc(Nc3ccccc3)c21)-c1ccccc1-4] | 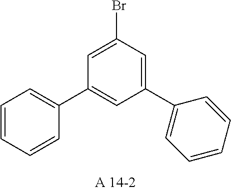 [SMILES: Brc1cc(-c2ccccc2)cc(-c2ccccc2)c1] | |
| 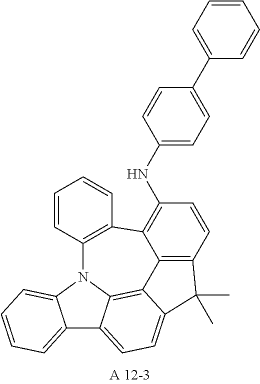 [SMILES: CC1(C)c2ccc(Nc3ccc(-c4ccccc4)cc3)c3c2-c2c1ccc1c4ccccc4n(c21)-c1ccccc1-3] | 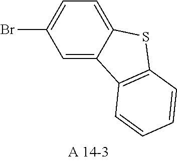 [SMILES: Brc1ccc2sc3ccccc3c2c1] | |
| 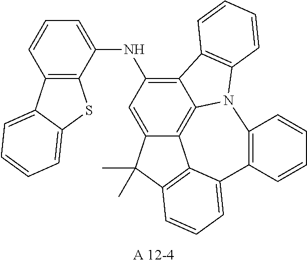 [SMILES: CC1(C)c2cccc3c2-c2c1cc(Nc1cccc4c1sc1ccccc14)c1c4ccccc4n(c21)-c1ccccc1-3] | 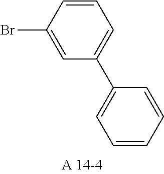 [SMILES: Brc1cccc(-c2ccccc2)c1] | |
| 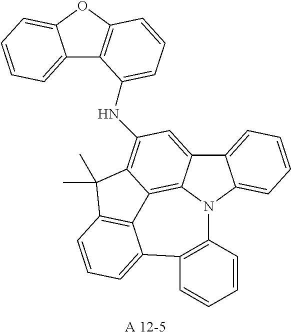 [SMILES: CC1(C)c2cccc3c2-c2c1c(Nc1cccc4oc5ccccc5c14)cc1c4ccccc4n(c21)-c1ccccc1-3] | 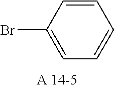 | |
| 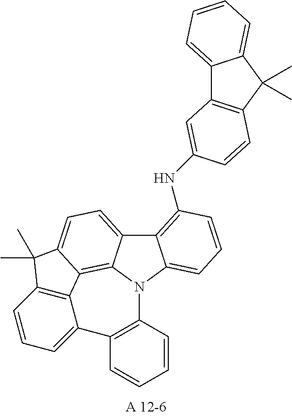 | 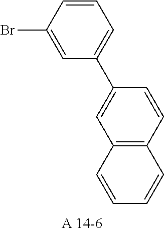 [SMILES: Brc1cccc(-c2ccc3ccccc3c2)c1] | |
| 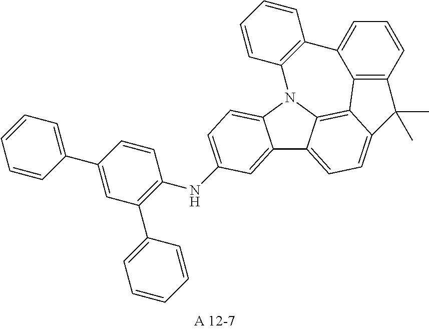 [SMILES: CC1(C)c2cccc3c2-c2c1ccc1c4cc(Nc5ccc(-c6ccccc6)cc5-c5ccccc5)ccc4n(c21)-c1ccccc1-3] | 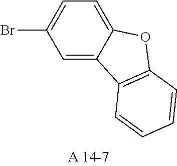 [SMILES: Brc1ccc2oc3ccccc3c2c1] | |
| 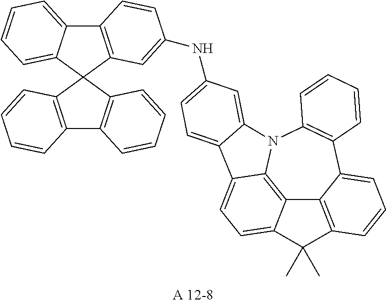 [SMILES: CC1(C)c2cccc3c2-c2c1ccc1c4ccc(Nc5ccc6c(c5)C5(c7ccccc7-c7ccccc75)c5ccccc5-6)cc4n(c21)-c1ccccc1-3] | 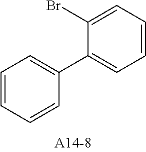 [SMILES: Brc1ccccc1-c1ccccc1] | |
| 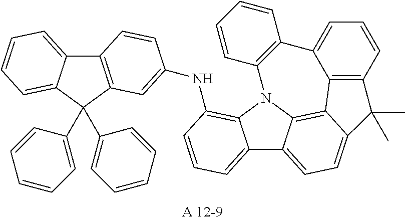 [SMILES: CC1(C)c2cccc3c2-c2c1ccc1c4cccc(Nc5ccc6c(c5)C(c5ccccc5)(c5ccccc5)c5ccccc5-6)c4n(c21)-c1ccccc1-3] | 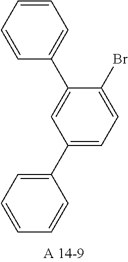 [SMILES: Brc1ccc(-c2ccccc2)cc1-c1ccccc1] | |
| 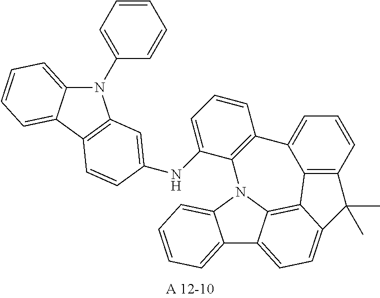 [SMILES: CC1(C)c2cccc3c2-c2c1ccc1c4ccccc4n(c21)-c1c(Nc2ccc4c5ccccc5n(-c5ccccc5)c4c2)cccc1-3] | 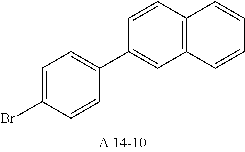 [SMILES: Brc1ccc(-c2ccc3ccccc3c2)cc1] | |
| 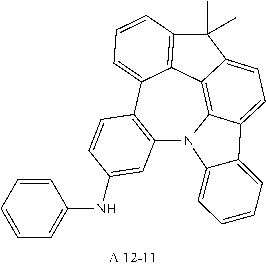 [SMILES: CC1(C)c2cccc3c2-c2c1ccc1c4ccccc4n(c21)-c1cc(Nc2ccccc2)ccc1-3] | 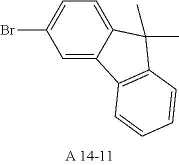 [SMILES: CC1(C)c2ccccc2-c2cc(Br)ccc21] | |
| 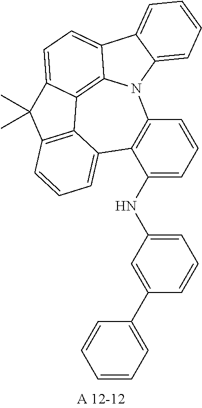 [SMILES: CC1(C)c2cccc3c2-c2c1ccc1c4ccccc4n(c21)-c1cccc(Nc2cccc(-c4ccccc4)c2)c1-3] | 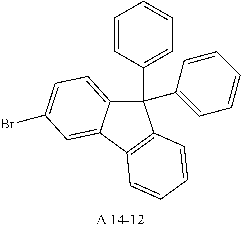 [SMILES: Brc1ccc2c(c1)-c1ccccc1C2(c1ccccc1)c1ccccc1] | |
| 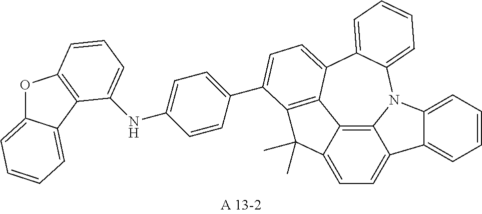 [SMILES: CC1(C)c2ccc3c4ccccc4n4c3c2-c2c(ccc(-c3ccc(Nc5cccc6oc7ccccc7c56)cc3)c21)-c1ccccc1-4] | 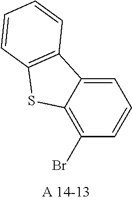 [SMILES: Brc1cccc2c1sc1ccccc12] | |
| 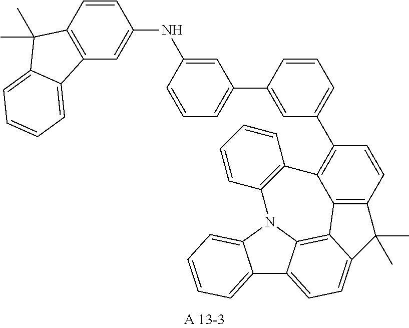 | 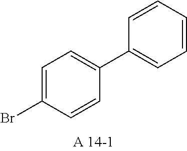 [SMILES: Brc1ccc(-c2ccccc2)cc1] | |
| 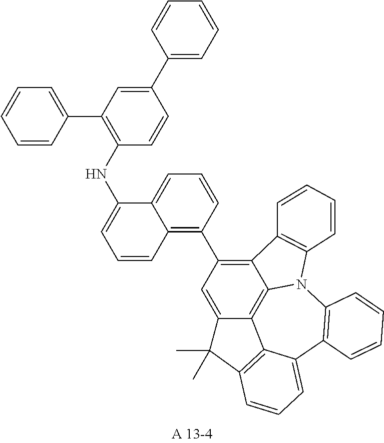 [SMILES: CC1(C)c2cccc3c2-c2c1cc(-c1cccc4c(Nc5ccc(-c6ccccc6)cc5-c5ccccc5)cccc14)c1c4ccccc4n(c21)-c1ccccc1-3] | 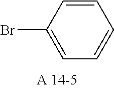 | |
| 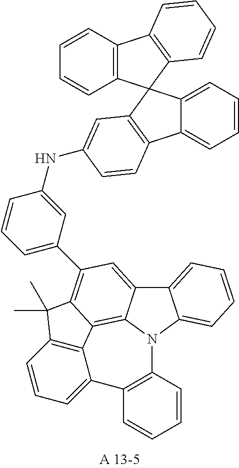 [SMILES: CC1(C)c2cccc3c2-c2c1c(-c1cccc(Nc4ccc5c(c4)C4(c6ccccc6-c6ccccc64)c4ccccc4-5)c1)cc1c4ccccc4n(c21)-c1ccccc1-3] | 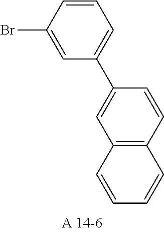 [SMILES: Brc1cccc(-c2ccc3ccccc3c2)c1] | |
| 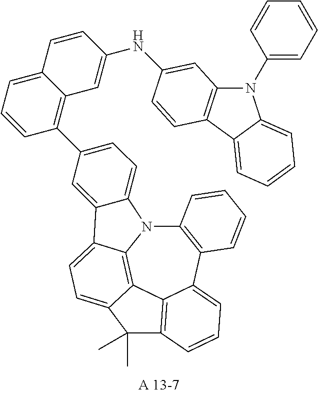 [SMILES: CC1(C)c2cccc3c2-c2c1ccc1c4cc(-c5cccc6ccc(Nc7ccc8c9ccccc9n(-c9ccccc9)c8c7)cc56)ccc4n(c21)-c1ccccc1-3] | 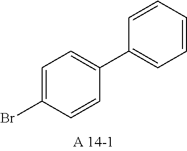 [SMILES: Brc1ccc(-c2ccccc2)cc1] | |
| 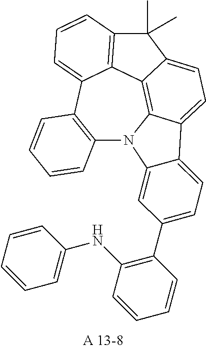 [SMILES: CC1(C)c2cccc3c2-c2c1ccc1c4ccc(-c5ccccc5Nc5ccccc5)cc4n(c21)-c1ccccc1-3] | 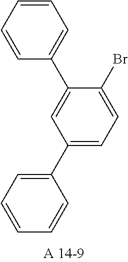 [SMILES: Brc1ccc(-c2ccccc2)cc1-c1ccccc1] | |
| 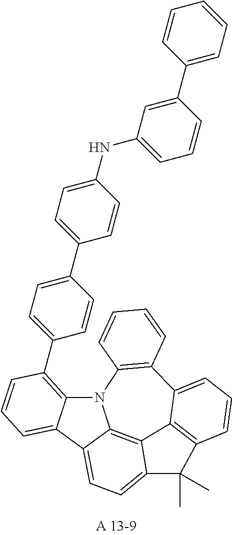 [SMILES: CC1(C)c2cccc3c2-c2c1ccc1c4cccc(-c5ccc(-c6ccc(Nc7cccc(-c8ccccc8)c7)cc6)cc5)c4n(c21)-c1ccccc1-3] | 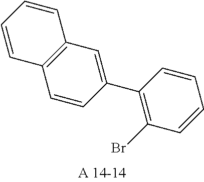 [SMILES: Brc1ccccc1-c1ccc2ccccc2c1] | |
| 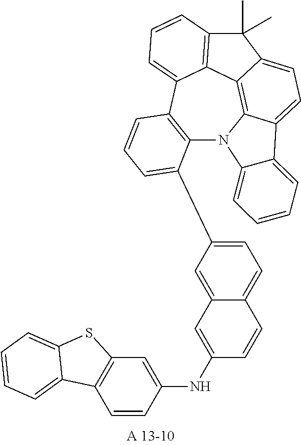 [SMILES: CC1(C)c2cccc3c2-c2c1ccc1c4ccccc4n(c21)-c1c(-c2ccc4ccc(Nc5ccc6c(c5)sc5ccccc56)cc4c2)cccc1-3] | 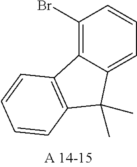 [SMILES: CC1(C)c2ccccc2-c2c(Br)cccc21] | |
| 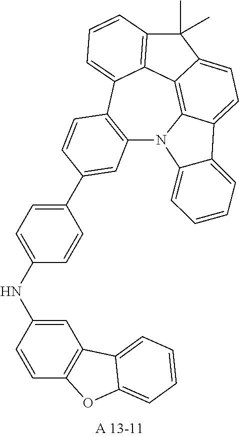 [SMILES: CC1(C)c2cccc3c2-c2c1ccc1c4ccccc4n(c21)-c1cc(-c2ccc(Nc4ccc5oc6ccccc6c5c4)cc2)ccc1-3] | 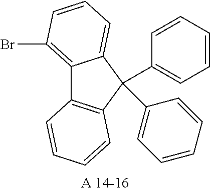 [SMILES: Brc1cccc2c1-c1ccccc1C2(c1ccccc1)c1ccccc1] | |
| 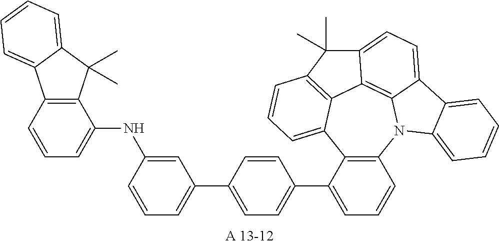 [SMILES: CC1(C)c2cccc3c2-c2c1ccc1c4ccccc4n(c21)-c1cccc(-c2ccc(-c4cccc(Nc5cccc6c5C(C)(C)c5ccccc5-6)c4)cc2)c1-3] | 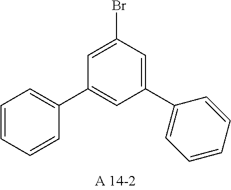 [SMILES: Brc1cc(-c2ccccc2)cc(-c2ccccc2)c1] | |
| 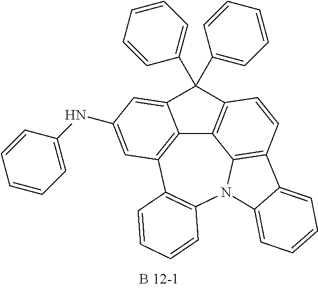 [SMILES: c1ccc(Nc2cc3c4c(c2)C(c2ccccc2)(c2ccccc2)c2ccc5c6ccccc6n(c5c2-4)-c2ccccc2-3)cc1] | 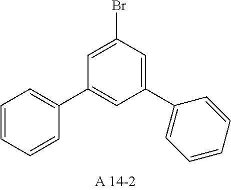 [SMILES: Brc1cc(-c2ccccc2)cc(-c2ccccc2)c1] | |
| 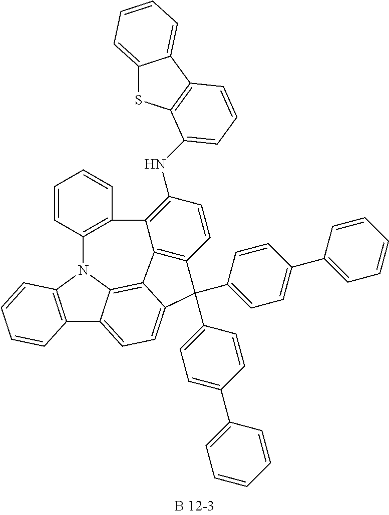 [SMILES: c1ccc(-c2ccc(C3(c4ccc(-c5ccccc5)cc4)c4ccc(Nc5cccc6c5sc5ccccc56)c5c4-c4c3ccc3c6ccccc6n(c43)-c3ccccc3-5)cc2)cc1] | 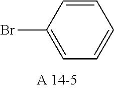 | |
| 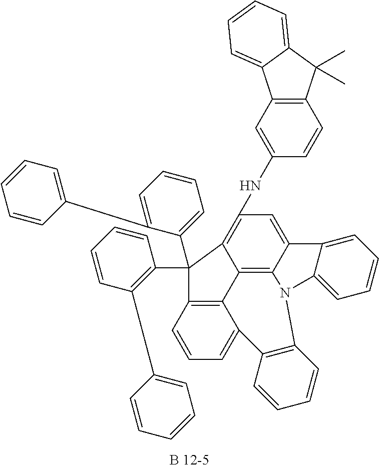 [SMILES: CC1(C)c2ccccc2-c2cc(Nc3cc4c5ccccc5n5c4c4c3C(c3ccccc3-c3ccccc3)(c3ccccc3-c3ccccc3)c3cccc(c3-4)-c3ccccc3-5)ccc21] | 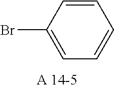 | |
| 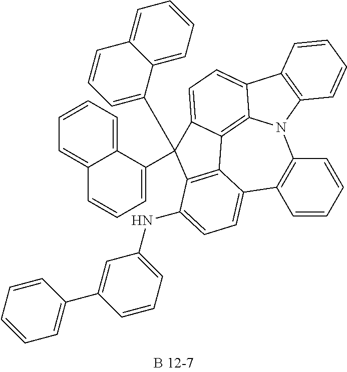 [SMILES: c1ccc(-c2cccc(Nc3ccc4c5c3C(c3cccc6ccccc36)(c3cccc6ccccc36)c3ccc6c7ccccc7n(c6c3-5)-c3ccccc3-4)c2)cc1] | 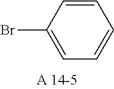 | |
| 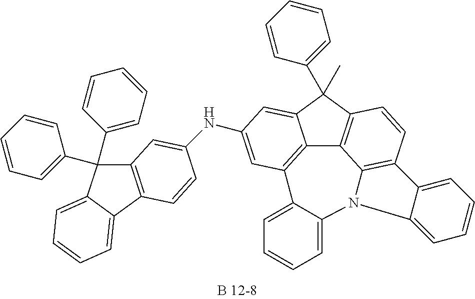 [SMILES: CC1(c2ccccc2)c2cc(Nc3ccc4c(c3)C(c3ccccc3)(c3ccccc3)c3ccccc3-4)cc3c2-c2c1ccc1c4ccccc4n(c21)-c1ccccc1-3] | 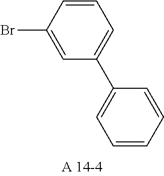 [SMILES: Brc1cccc(-c2ccccc2)c1] | |
| 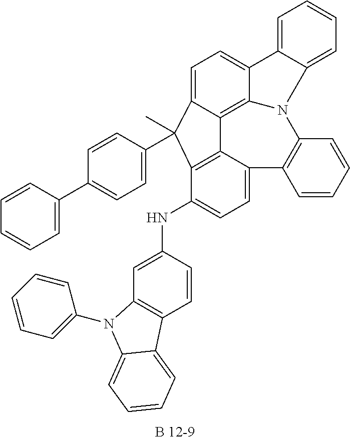 [SMILES: CC1(c2ccc(-c3ccccc3)cc2)c2ccc3c4ccccc4n4c3c2-c2c(ccc(Nc3ccc5c6ccccc6n(-c6ccccc6)c5c3)c21)-c1ccccc1-4] | 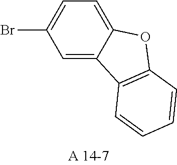 [SMILES: Brc1ccc2oc3ccccc3c2c1] | |
| 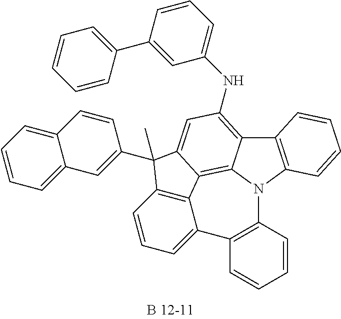 [SMILES: CC1(c2ccc3ccccc3c2)c2cccc3c2-c2c1cc(Nc1cccc(-c4ccccc4)c1)c1c4ccccc4n(c21)-c1ccccc1-3] | 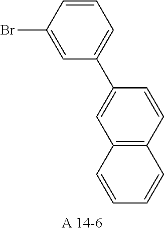 [SMILES: Brc1cccc(-c2ccc3ccccc3c2)c1] | |
| 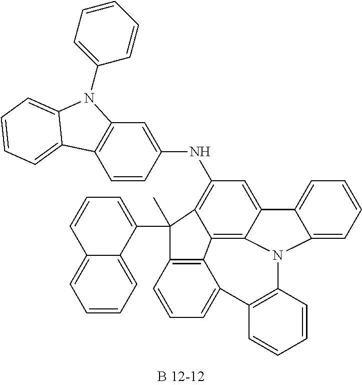 [SMILES: CC1(c2cccc3ccccc23)c2cccc3c2-c2c1c(Nc1ccc4c5ccccc5n(-c5ccccc5)c4c1)cc1c4ccccc4n(c21)-c1ccccc1-3] | 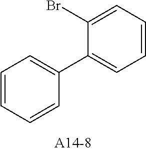 [SMILES: Brc1ccccc1-c1ccccc1] | |
| 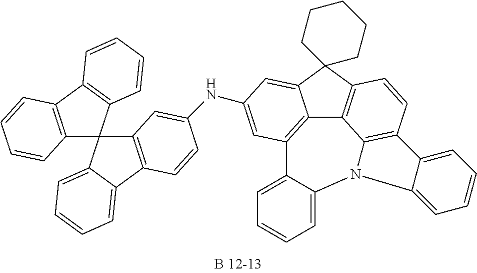 [SMILES: c1ccc2c(c1)-c1cc(Nc3ccc4c(c3)C3(c5ccccc5-c5ccccc53)c3ccccc3-4)cc3c1-c1c(ccc4c5ccccc5n-2c14)C31CCCCC1] | 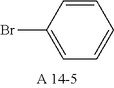 | |
| 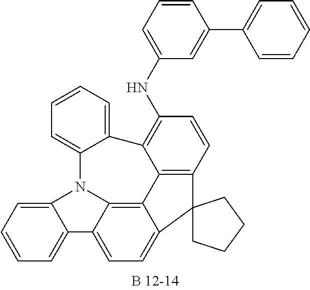 [SMILES: c1ccc(-c2cccc(Nc3ccc4c5c3-c3ccccc3-n3c6ccccc6c6ccc(c-5c63)C43CCCC3)c2)cc1] | 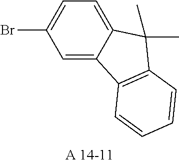 [SMILES: CC1(C)c2ccccc2-c2cc(Br)ccc21] | |
| 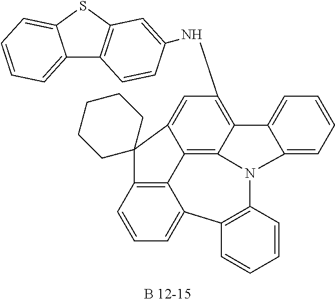 [SMILES: c1ccc2c(c1)-c1cccc3c1-c1c(cc(Nc4ccc5c(c4)sc4ccccc45)c4c5ccccc5n-2c14)C31CCCCC1] | 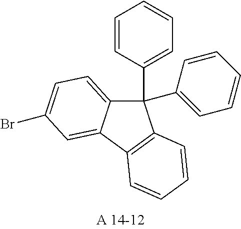 [SMILES: Brc1ccc2c(c1)-c1ccccc1C2(c1ccccc1)c1ccccc1] | |
| 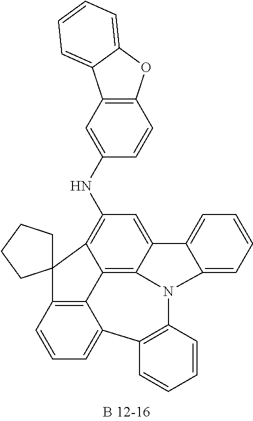 [SMILES: c1ccc2c(c1)-c1cccc3c1-c1c(c(Nc4ccc5oc6ccccc6c5c4)cc4c5ccccc5n-2c14)C31CCCC1] | 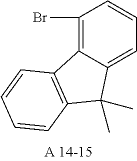 [SMILES: CC1(C)c2ccccc2-c2c(Br)cccc21] | |
| 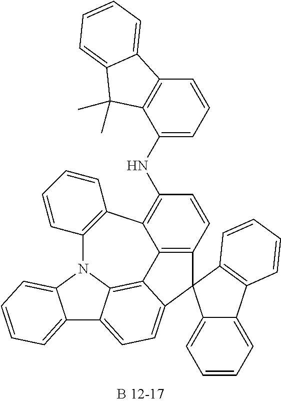 [SMILES: CC1(C)c2ccccc2-c2cccc(Nc3ccc4c5c3-c3ccccc3-n3c6ccccc6c6ccc(c-5c63)C43c4ccccc4-c4ccccc43)c21] | 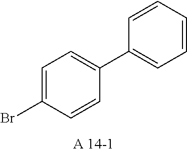 [SMILES: Brc1ccc(-c2ccccc2)cc1] | |
| 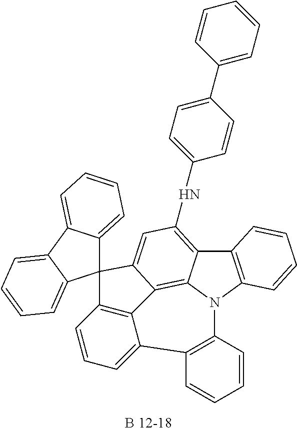 [SMILES: c1ccc(-c2ccc(Nc3cc4c5c6c3c3ccccc3n6-c3ccccc3-c3cccc(c3-5)C43c4ccccc4-c4ccccc43)cc2)cc1] | 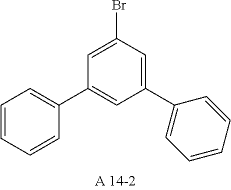 [SMILES: Brc1cc(-c2ccccc2)cc(-c2ccccc2)c1] | |
| 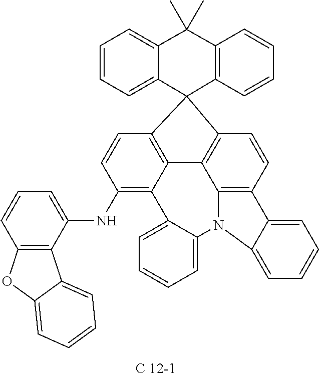 [SMILES: CC1(C)c2ccccc2C2(c3ccccc31)c1ccc(Nc3cccc4oc5ccccc5c34)c3c1-c1c2ccc2c4ccccc4n(c12)-c1ccccc1-3] | 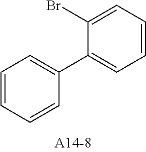 [SMILES: Brc1ccccc1-c1ccccc1] | |
| 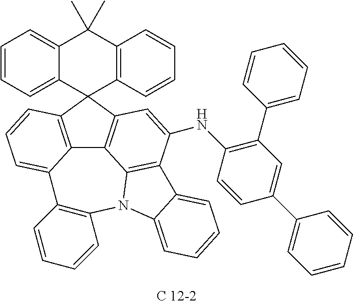 [SMILES: CC1(C)c2ccccc2C2(c3ccccc31)c1cccc3c1-c1c2cc(Nc2ccc(-c4ccccc4)cc2-c2ccccc2)c2c4ccccc4n(c12)-c1ccccc1-3] | 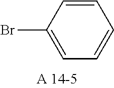 | |
| 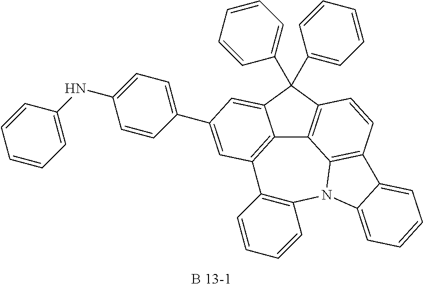 [SMILES: c1ccc(Nc2ccc(-c3cc4c5c(c3)C(c3ccccc3)(c3ccccc3)c3ccc6c7ccccc7n(c6c3-5)-c3ccccc3-4)cc2)cc1] | 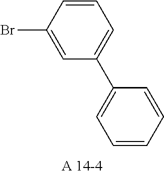 [SMILES: Brc1cccc(-c2ccccc2)c1] | |
| 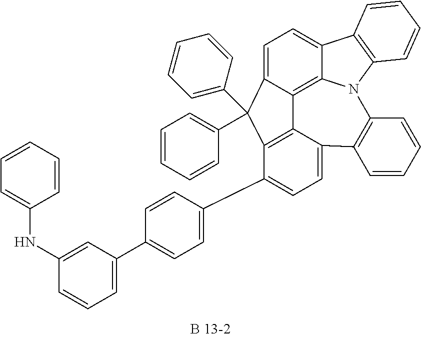 [SMILES: c1ccc(Nc2cccc(-c3ccc(-c4ccc5c6c4C(c4ccccc4)(c4ccccc4)c4ccc7c8ccccc8n(c7c4-6)-c4ccccc4-5)cc3)c2)cc1] | 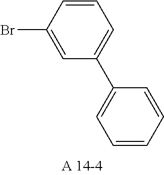 [SMILES: Brc1cccc(-c2ccccc2)c1] | |
| 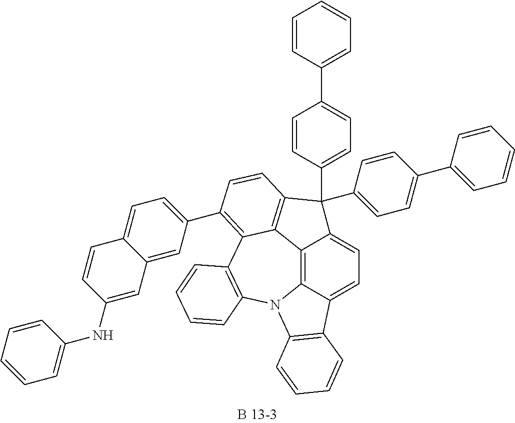 [SMILES: c1ccc(Nc2ccc3ccc(-c4ccc5c6c4-c4ccccc4-n4c7ccccc7c7ccc(c-6c74)C5(c4ccc(-c5ccccc5)cc4)c4ccc(-c5ccccc5)cc4)cc3c2)cc1] | 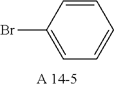 | |
| 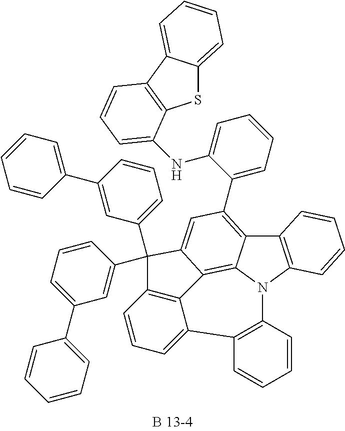 [SMILES: c1ccc(-c2cccc(C3(c4cccc(-c5ccccc5)c4)c4cccc5c4-c4c3cc(-c3ccccc3Nc3cccc6c3sc3ccccc36)c3c6ccccc6n(c43)-c3ccccc3-5)c2)cc1] | 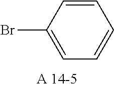 | |
| 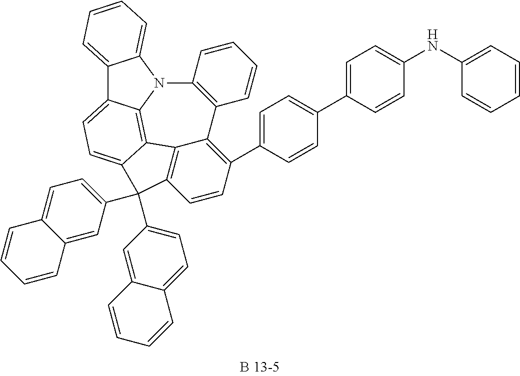 [SMILES: c1ccc(Nc2ccc(-c3ccc(-c4ccc5c6c4-c4ccccc4-n4c7ccccc7c7ccc(c-6c74)C5(c4ccc5ccccc5c4)c4ccc5ccccc5c4)cc3)cc2)cc1] | 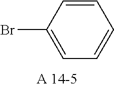 | |
| 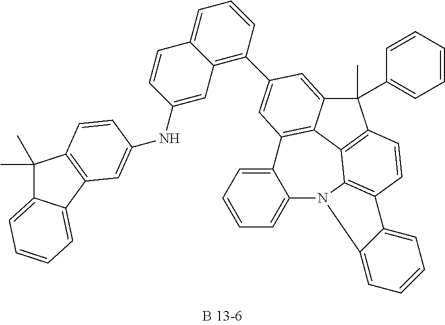 [SMILES: CC1(C)c2ccccc2-c2cc(Nc3ccc4cccc(-c5cc6c7c(c5)C(C)(c5ccccc5)c5ccc8c9ccccc9n(c8c5-7)-c5ccccc5-6)c4c3)ccc21] | 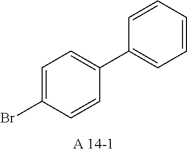 [SMILES: Brc1ccc(-c2ccccc2)cc1] | |
| 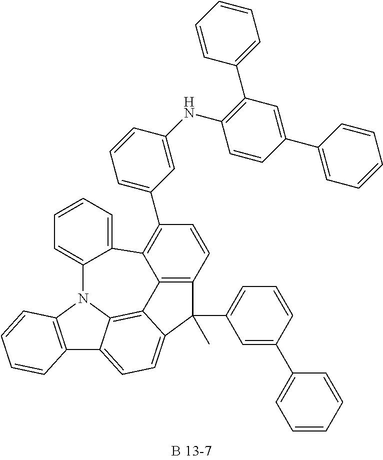 [SMILES: CC1(c2cccc(-c3ccccc3)c2)c2ccc(-c3cccc(Nc4ccc(-c5ccccc5)cc4-c4ccccc4)c3)c3c2-c2c1ccc1c4ccccc4n(c21)-c1ccccc1-3] | 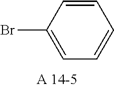 | |
| 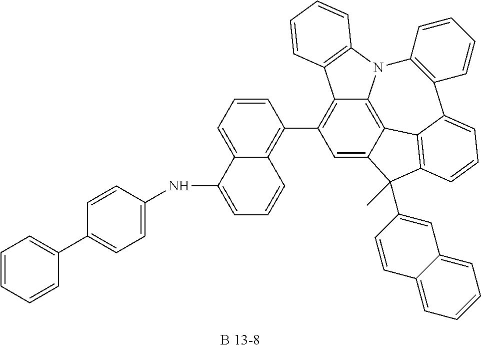 [SMILES: CC1(c2ccc3ccccc3c2)c2cccc3c2-c2c1cc(-c1cccc4c(Nc5ccc(-c6ccccc6)cc5)cccc14)c1c4ccccc4n(c21)-c1ccccc1-3] | 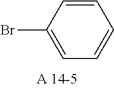 | |
| 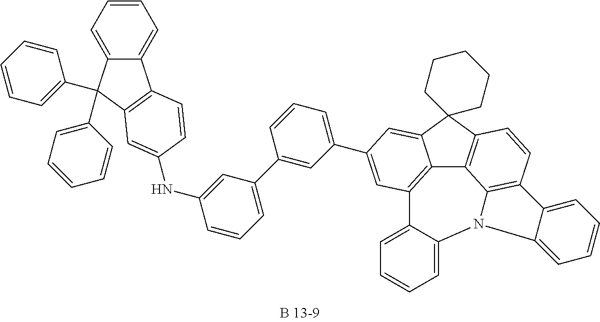 [SMILES: c1ccc(C2(c3ccccc3)c3ccccc3-c3ccc(Nc4cccc(-c5cccc(-c6cc7c8c(c6)C6(CCCCC6)c6ccc9c%10ccccc%10n(c9c6-8)-c6ccccc6-7)c5)c4)cc32)cc1] | 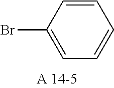 | |
| 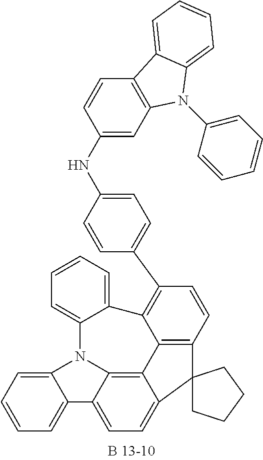 [SMILES: c1ccc(-n2c3ccccc3c3ccc(Nc4ccc(-c5ccc6c7c5-c5ccccc5-n5c8ccccc8c8ccc(c-7c85)C65CCCC5)cc4)cc32)cc1] | 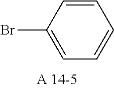 | |
| 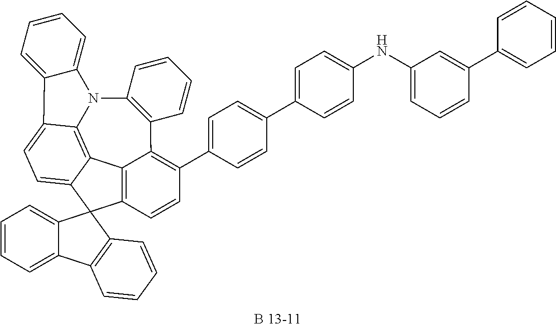 [SMILES: c1ccc(-c2cccc(Nc3ccc(-c4ccc(-c5ccc6c7c5-c5ccccc5-n5c8ccccc8c8ccc(c-7c85)C65c6ccccc6-c6ccccc65)cc4)cc3)c2)cc1] | 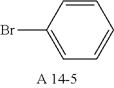 | |
| 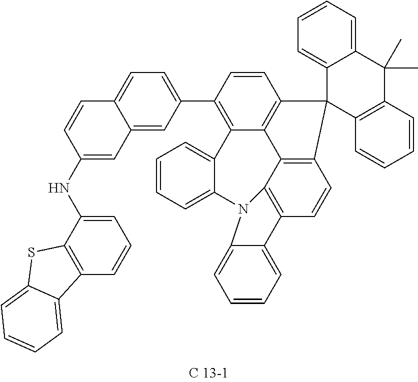 [SMILES: CC1(C)c2ccccc2C2(c3ccccc31)c1ccc(-c3ccc4ccc(Nc5cccc6c5sc5ccccc56)cc4c3)c3c1-c1c2ccc2c4ccccc4n(c12)-c1ccccc1-3] | 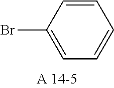 | |
| | | MS |
| Compound X | Yield (%) | [M + H]⁺ |
| 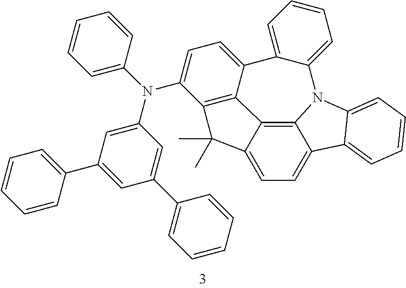 [SMILES: CC1(C)c2ccc3c4ccccc4n4c3c2-c2c(ccc(N(c3ccccc3)c3cc(-c5ccccc5)cc(-c5ccccc5)c3)c21)-c1ccccc1-4] | 63.5 | 677.3 |
| 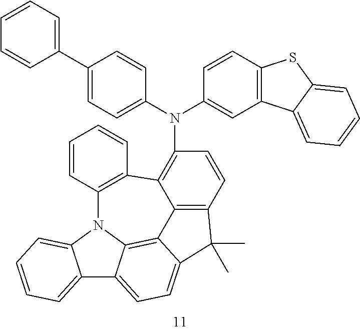 [SMILES: CC1(C)c2ccc(N(c3ccc(-c4ccccc4)cc3)c3ccc4sc5ccccc5c4c3)c3c2-c2c1ccc1c4ccccc4n(c21)-c1ccccc1-3] | 58.9 | 707.2 |
| 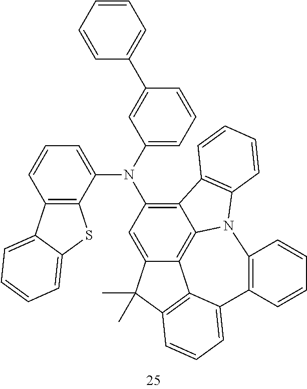 [SMILES: CC1(C)c2cccc3c2-c2c1cc(N(c1cccc(-c4ccccc4)c1)c1cccc4c1sc1ccccc14)c1c4ccccc4n(c21)-c1ccccc1-3] | 66.1 | 707.2 |
| 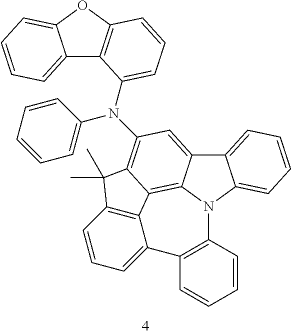 [SMILES: CC1(C)c2cccc3c2-c2c1c(N(c1ccccc1)c1cccc4oc5ccccc5c14)cc1c4ccccc4n(c21)-c1ccccc1-3] | 70.5 | 615.2 |
| 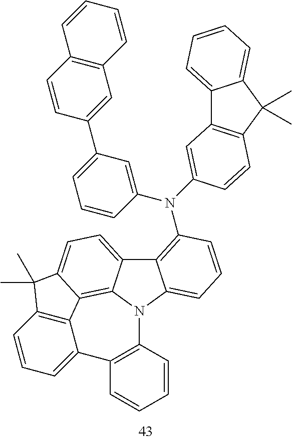 | 64.4 | 767.3 |
| 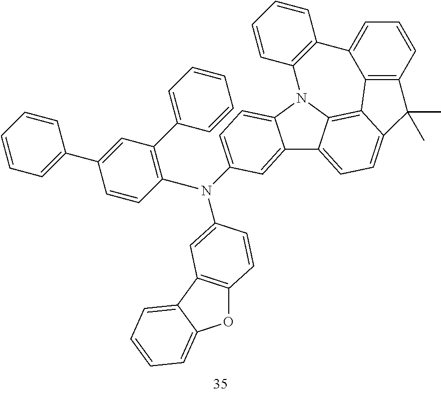 [SMILES: CC1(C)c2cccc3c2-c2c1ccc1c4cc(N(c5ccc6oc7ccccc7c6c5)c5ccc(-c6ccccc6)cc5-c5ccccc5)ccc4n(c21)-c1ccccc1-3] | 57.8 | 767.3 |
| 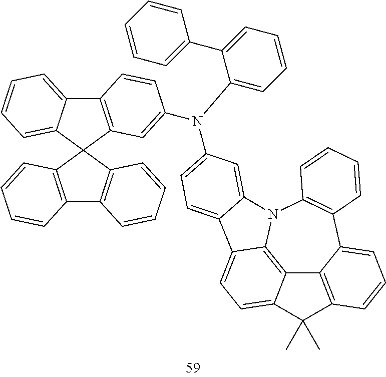 [SMILES: CC1(C)c2cccc3c2-c2c1ccc1c4ccc(N(c5ccc6c(c5)C5(c7ccccc7-c7ccccc75)c5ccccc5-6)c5ccccc5-c5ccccc5)cc4n(c21)-c1ccccc1-3] | 60.2 | 839.3 |
| 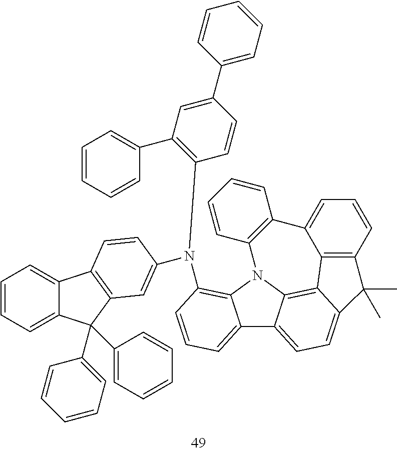 [SMILES: CC1(C)c2cccc3c2-c2c1ccc1c4cccc(N(c5ccc6c(c5)C(c5ccccc5)(c5ccccc5)c5ccccc5-6)c5ccc(-c6ccccc6)cc5-c5ccccc5)c4n(c21)-c1ccccc1-3] | 55.6 | 917.3 |
| 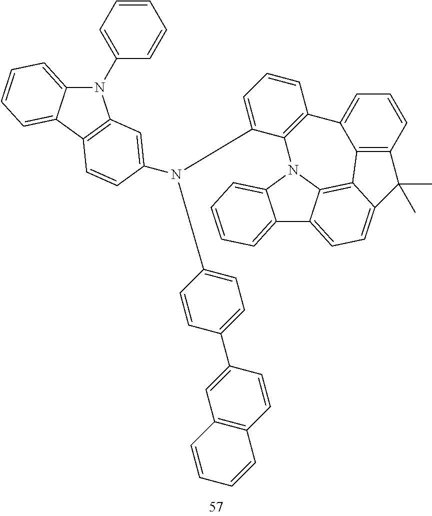 [SMILES: CC1(C)c2cccc3c2-c2c1ccc1c4ccccc4n(c21)-c1c-3cccc1N(c1ccc(-c2ccc3ccccc3c2)cc1)c1ccc2c3ccccc3n(-c3ccccc3)c2c1] | 51.1 | 816.3 |
| 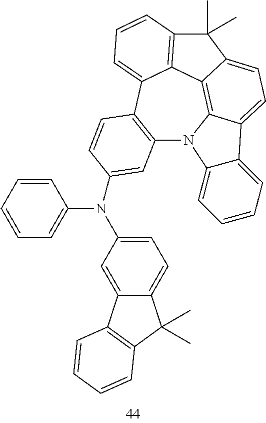 [SMILES: CC1(C)c2ccccc2-c2cc(N(c3ccccc3)c3ccc4c(c3)-n3c5ccccc5c5ccc6c(c53)-c3c-4cccc3C6(C)C)ccc21] | 68.2 | 641.3 |
| 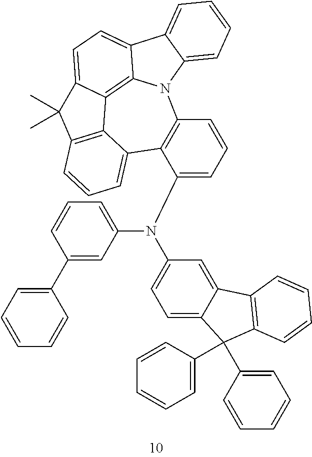 [SMILES: CC1(C)c2cccc3c2-c2c1ccc1c4ccccc4n(c21)-c1cccc(N(c2cccc(-c4ccccc4)c2)c2ccc4c(c2)-c2ccccc2C4(c2ccccc2)c2ccccc2)c1-3] | 56.6 | 841.4 |
| 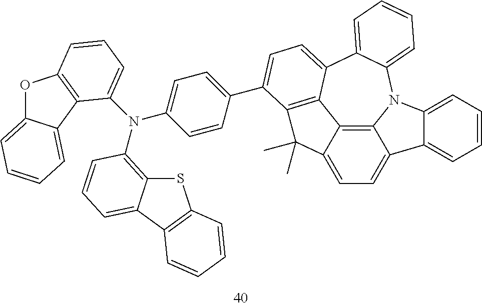 [SMILES: CC1(C)c2ccc3c4ccccc4n4c3c2-c2c(ccc(-c3ccc(N(c5cccc6c5sc5ccccc56)c5cccc6oc7ccccc7c56)cc3)c21)-c1ccccc1-4] | 60.5 | 797.3 |
| 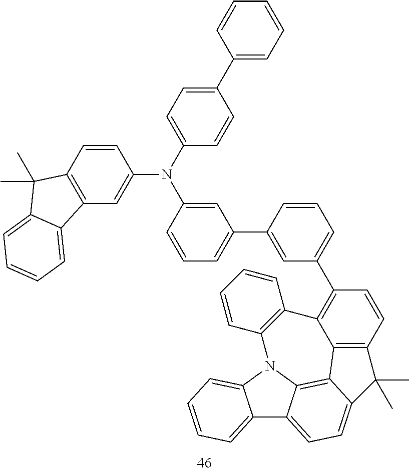 | 66.8 | 869.4 |
| 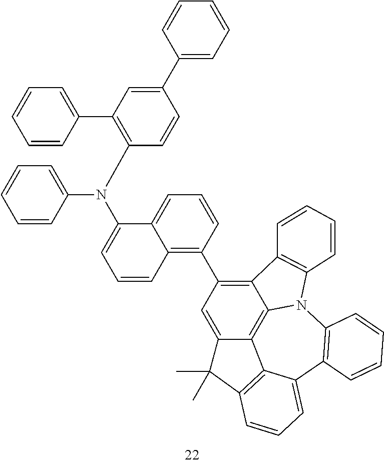 [SMILES: CC1(C)c2cccc3c2-c2c1cc(-c1cccc4c(N(c5ccccc5)c5ccc(-c6ccccc6)cc5-c5ccccc5)cccc14)c1c4ccccc4n(c21)-c1ccccc1-3] | 70.7 | 803.3 |
| 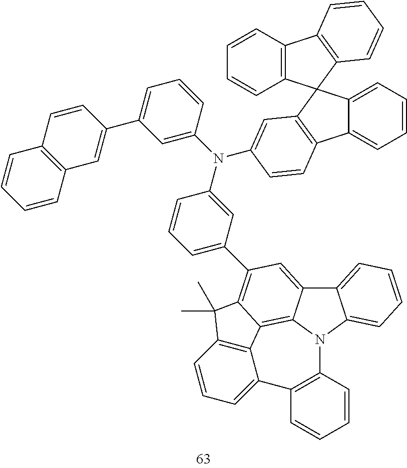 [SMILES: CC1(C)c2cccc3c2-c2c1c(-c1cccc(N(c4cccc(-c5ccc6ccccc6c5)c4)c4ccc5c(c4)C4(c6ccccc6-c6ccccc64)c4ccccc4-5)c1)cc1c4ccccc4n(c21)-c1ccccc1-3] | 62.3 | 965.4 |
| 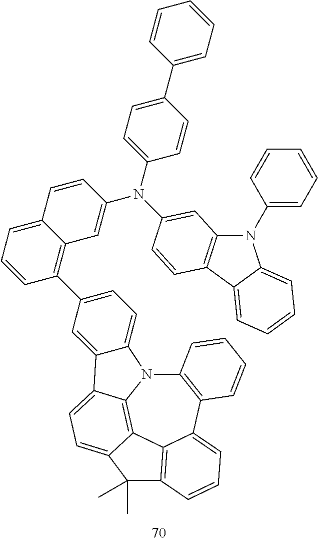 [SMILES: CC1(C)c2cccc3c2-c2c1ccc1c4cc(-c5cccc6ccc(N(c7ccc(-c8ccccc8)cc7)c7ccc8c9ccccc9n(-c9ccccc9)c8c7)cc56)ccc4n(c21)-c1ccccc1-3] | 68.1 | 892.4 |
| 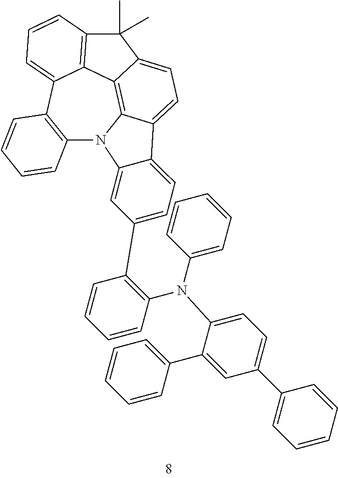 [SMILES: CC1(C)c2cccc3c2-c2c1ccc1c4ccc(-c5ccccc5N(c5ccccc5)c5ccc(-c6ccccc6)cc5-c5ccccc5)cc4n(c21)-c1ccccc1-3] | 61.6 | 753.3 |
| 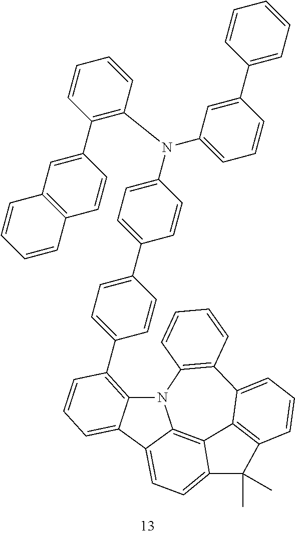 [SMILES: CC1(C)c2cccc3c2-c2c1ccc1c4cccc(-c5ccc(-c6ccc(N(c7cccc(-c8ccccc8)c7)c7ccccc7-c7ccc8ccccc8c7)cc6)cc5)c4n(c21)-c1ccccc1-3] | 66.1 | 879.4 |
| 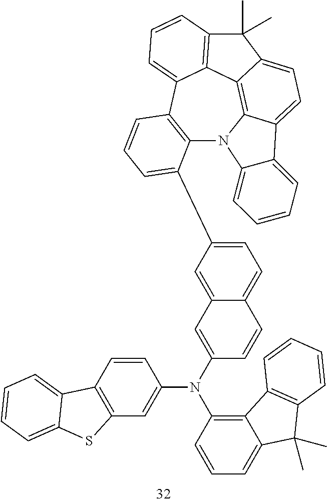 [SMILES: CC1(C)c2ccccc2-c2c(N(c3ccc4ccc(-c5cccc6c5-n5c7ccccc7c7ccc8c(c75)-c5c-6cccc5C8(C)C)cc4c3)c3ccc4c(c3)sc3ccccc34)cccc21] | 60.5 | 873.3 |
| 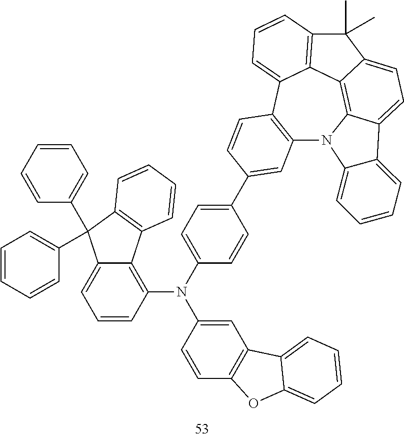 [SMILES: CC1(C)c2cccc3c2-c2c1ccc1c4ccccc4n(c21)-c1cc(-c2ccc(N(c4ccc5oc6ccccc6c5c4)c4cccc5c4-c4ccccc4C5(c4ccccc4)c4ccccc4)cc2)ccc1-3] | 59.8 | 931.4 |
| 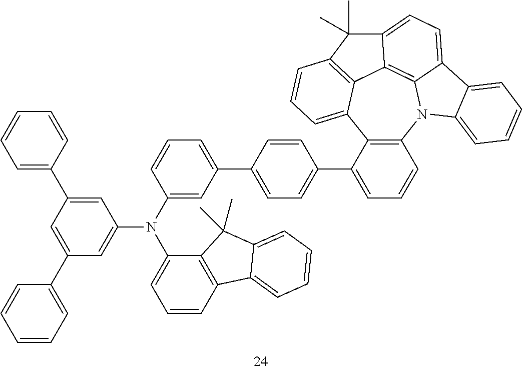 [SMILES: CC1(C)c2cccc3c2-c2c1ccc1c4ccccc4n(c21)-c1cccc(-c2ccc(-c4cccc(N(c5cc(-c6ccccc6)cc(-c6ccccc6)c5)c5cccc6c5C(C)(C)c5ccccc5-6)c4)cc2)c1-3] | 62.6 | 945.4 |
| 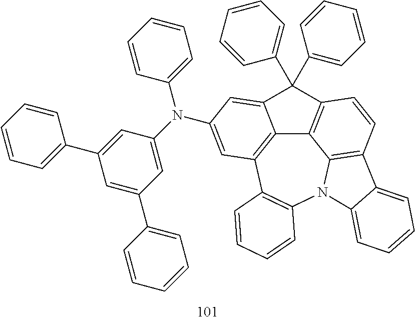 [SMILES: c1ccc(-c2cc(-c3ccccc3)cc(N(c3ccccc3)c3cc4c5c(c3)C(c3ccccc3)(c3ccccc3)c3ccc6c7ccccc7n(c6c3-5)-c3ccccc3-4)c2)cc1] | 65.9 | 801.3 |
| 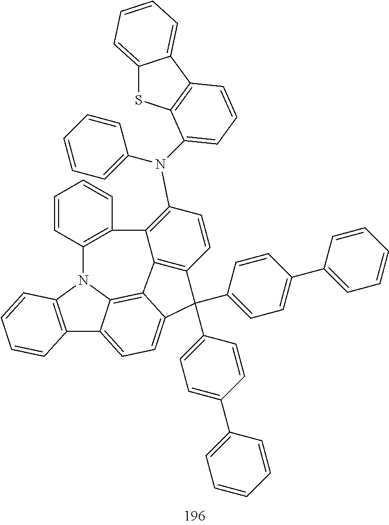 [SMILES: c1ccc(-c2ccc(C3(c4ccc(-c5ccccc5)cc4)c4ccc(N(c5ccccc5)c5cccc6c5sc5ccccc56)c5c4-c4c3ccc3c6ccccc6n(c43)-c3ccccc3-5)cc2)cc1] | 62.1 | 907.3 |
| 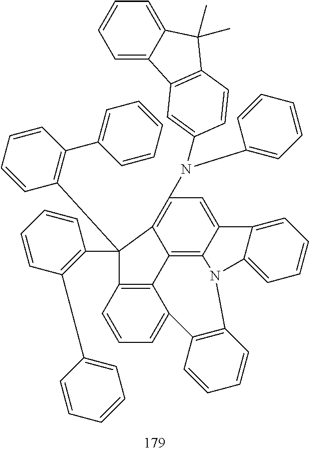 [SMILES: CC1(C)c2ccccc2-c2cc(N(c3ccccc3)c3cc4c5ccccc5n5c4c4c3C(c3ccccc3-c3ccccc3)(c3ccccc3-c3ccccc3)c3cccc(c3-4)-c3ccccc3-5)ccc21] | 58.4 | 917.4 |
| 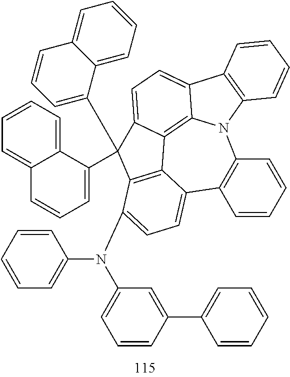 [SMILES: c1ccc(-c2cccc(N(c3ccccc3)c3ccc4c5c3C(c3cccc6ccccc36)(c3cccc6ccccc36)c3ccc6c7ccccc7n(c6c3-5)-c3ccccc3-4)c2)cc1] | 56.3 | 825.3 |
| 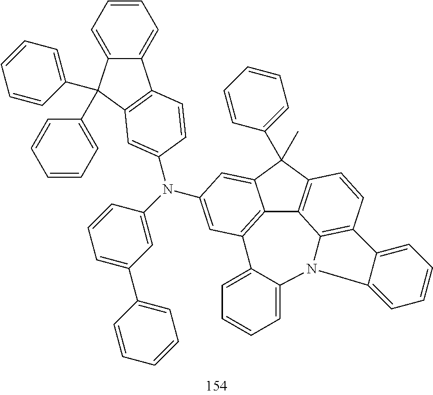 [SMILES: CC1(c2ccccc2)c2cc(N(c3cccc(-c4ccccc4)c3)c3ccc4c(c3)C(c3ccccc3)(c3ccccc3)c3ccccc3-4)cc3c2-c2c1ccc1c4ccccc4n(c21)-c1ccccc1-3] | 65.2 | 903.4 |
| 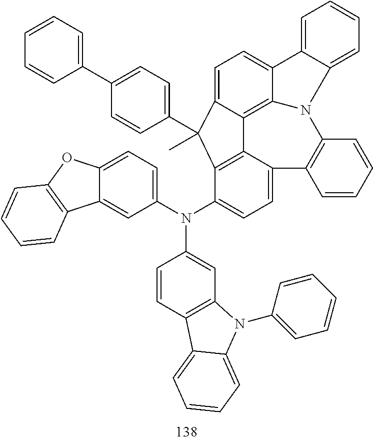 [SMILES: CC1(c2ccc(-c3ccccc3)cc2)c2ccc3c4ccccc4n4c3c2-c2c(ccc(N(c3ccc5oc6ccccc6c5c3)c3ccc5c6ccccc6n(-c6ccccc6)c5c3)c21)-c1ccccc1-4] | 60.1 | 918.3 |
| 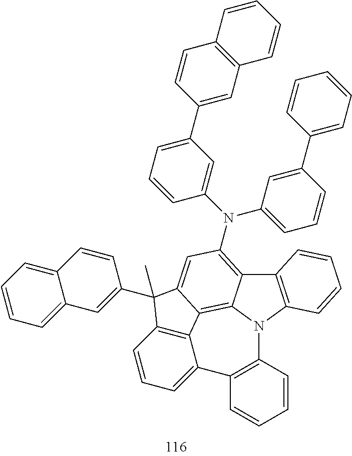 [SMILES: CC1(c2ccc3ccccc3c2)c2cccc3c2-c2c1cc(N(c1cccc(-c4ccccc4)c1)c1cccc(-c4ccc5ccccc5c4)c1)c1c4ccccc4n(c21)-c1ccccc1-3] | 63.4 | 839.3 |
| 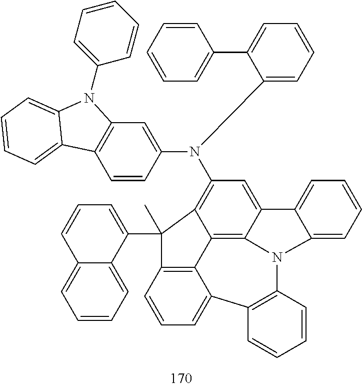 [SMILES: CC1(c2cccc3ccccc23)c2cccc3c2-c2c1c(N(c1ccc4c5ccccc5n(-c5ccccc5)c4c1)c1ccccc1-c1ccccc1)cc1c4ccccc4n(c21)-c1ccccc1-3] | 60.2 | 878.3 |
| 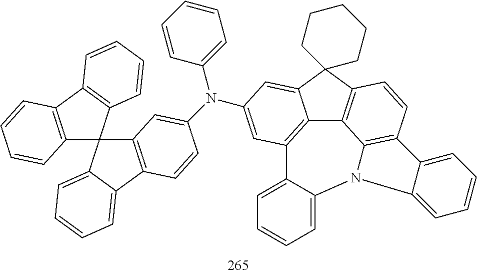 [SMILES: c1ccc(N(c2cc3c4c(c2)C2(CCCCC2)c2ccc5c6ccccc6n(c5c2-4)-c2ccccc2-3)c2ccc3c(c2)C2(c4ccccc4-c4ccccc42)c2ccccc2-3)cc1] | 68.1 | 803.3 |
| 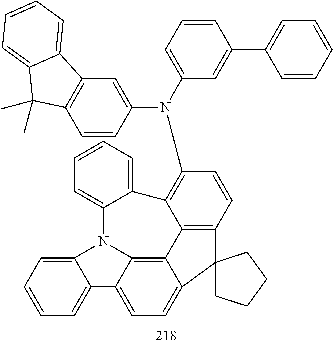 [SMILES: CC1(C)c2ccccc2-c2cc(N(c3cccc(-c4ccccc4)c3)c3ccc4c5c3-c3ccccc3-n3c6ccccc6c6ccc(c-5c63)C43CCCC3)ccc21] | 62.3 | 743.3 |
| 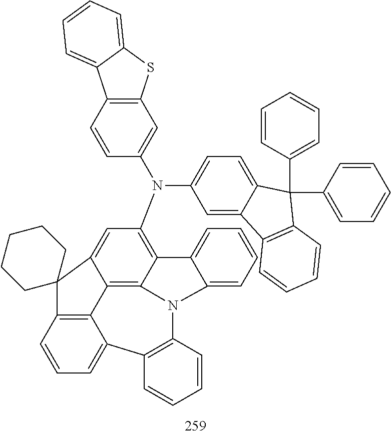 [SMILES: c1ccc(C2(c3ccccc3)c3ccccc3-c3cc(N(c4ccc5c(c4)sc4ccccc45)c4cc5c6c7c4c4ccccc4n7-c4ccccc4-c4cccc(c4-6)C54CCCCC4)ccc32)cc1] | 66.1 | 911.3 |
| 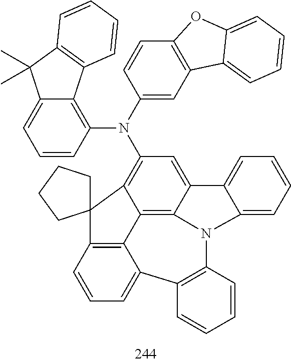 [SMILES: CC1(C)c2ccccc2-c2c(N(c3ccc4oc5ccccc5c4c3)c3cc4c5ccccc5n5c4c4c3C3(CCCC3)c3cccc(c3-4)-c3ccccc3-5)cccc21] | 61.7 | 757.3 |
| 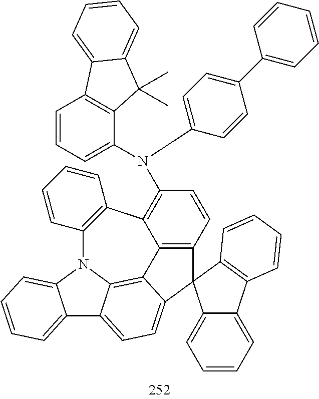 [SMILES: CC1(C)c2ccccc2-c2cccc(N(c3ccc(-c4ccccc4)cc3)c3ccc4c5c3-c3ccccc3-n3c6ccccc6c6ccc(c-5c63)C43c4ccccc4-c4ccccc43)c21] | 64.8 | 839.3 |
| 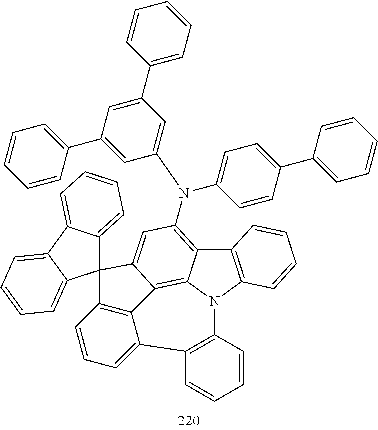 [SMILES: c1ccc(-c2ccc(N(c3cc(-c4ccccc4)cc(-c4ccccc4)c3)c3cc4c5c6c3c3ccccc3n6-c3ccccc3-c3cccc(c3-5)C43c4ccccc4-c4ccccc43)cc2)cc1] | 65.7 | 875.3 |
| 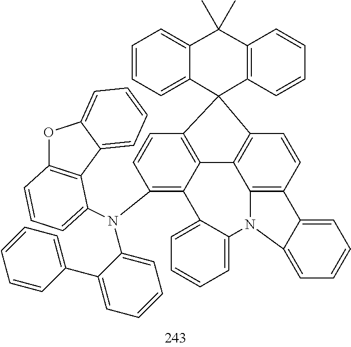 [SMILES: CC1(C)c2ccccc2C2(c3ccccc31)c1ccc(N(c3ccccc3-c3ccccc3)c3cccc4oc5ccccc5c34)c3c1-c1c2ccc2c4ccccc4n(c12)-c1ccccc1-3] | 60.3 | 855.3 |
| 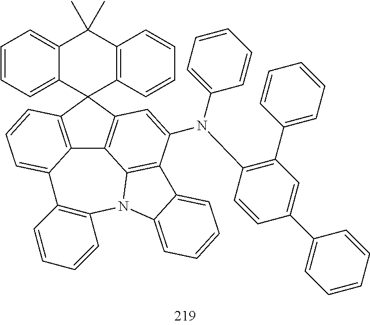 [SMILES: CC1(C)c2ccccc2C2(c3ccccc31)c1cccc3c1-c1c2cc(N(c2ccccc2)c2ccc(-c4ccccc4)cc2-c2ccccc2)c2c4ccccc4n(c12)-c1ccccc1-3] | 65.9 | 841.3 |
| 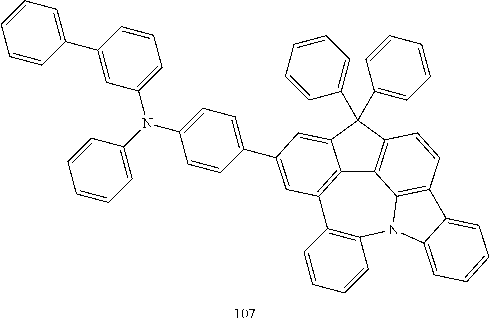 [SMILES: c1ccc(-c2cccc(N(c3ccccc3)c3ccc(-c4cc5c6c(c4)C(c4ccccc4)(c4ccccc4)c4ccc7c8ccccc8n(c7c4-6)-c4ccccc4-5)cc3)c2)cc1] | 66.8 | 801.3 |
| 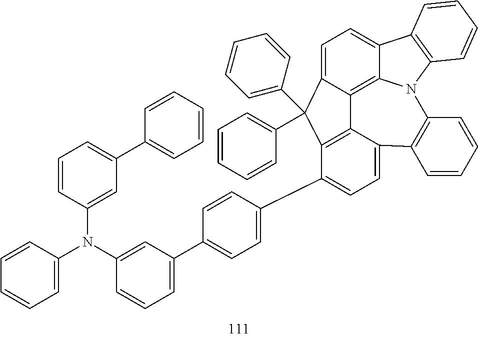 [SMILES: c1ccc(-c2cccc(N(c3ccccc3)c3cccc(-c4ccc(-c5ccc6c7c5C(c5ccccc5)(c5ccccc5)c5ccc8c9ccccc9n(c8c5-7)-c5ccccc5-6)cc4)c3)c2)cc1] | 60.1 | 877.3 |
| 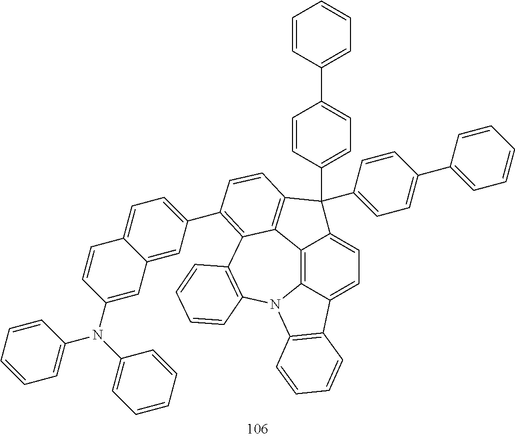 [SMILES: c1ccc(-c2ccc(C3(c4ccc(-c5ccccc5)cc4)c4ccc(-c5ccc6ccc(N(c7ccccc7)c7ccccc7)cc6c5)c5c4-c4c3ccc3c6ccccc6n(c43)-c3ccccc3-5)cc2)cc1] | 66.2 | 927.4 |
| 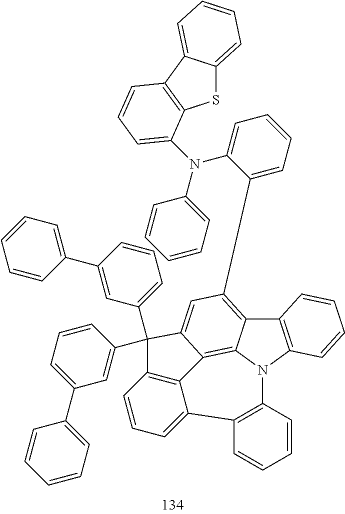 [SMILES: c1ccc(-c2cccc(C3(c4cccc(-c5ccccc5)c4)c4cccc5c4-c4c3cc(-c3ccccc3N(c3ccccc3)c3cccc6c3sc3ccccc36)c3c6ccccc6n(c43)-c3ccccc3-5)c2)cc1] | 59.8 | 983.3 |
| 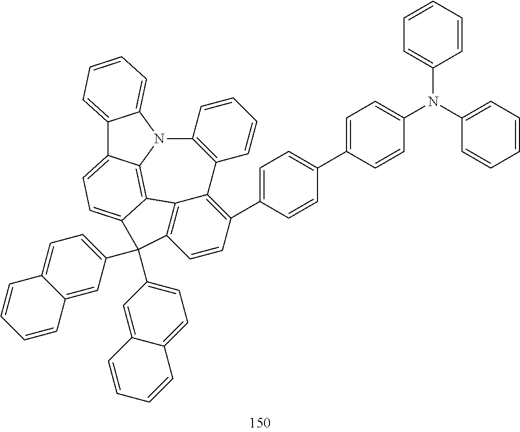 [SMILES: c1ccc(N(c2ccccc2)c2ccc(-c3ccc(-c4ccc5c6c4-c4ccccc4-n4c7ccccc7c7ccc(c-6c74)C5(c4ccc5ccccc5c4)c4ccc5ccccc5c4)cc3)cc2)cc1] | 65.4 | 901.3 |
| 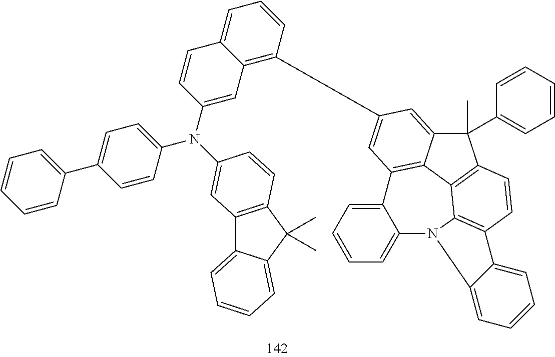 [SMILES: CC1(C)c2ccccc2-c2cc(N(c3ccc(-c4ccccc4)cc3)c3ccc4cccc(-c5cc6c7c(c5)C(C)(c5ccccc5)c5ccc8c9ccccc9n(c8c5-7)-c5ccccc5-6)c4c3)ccc21] | 62.3 | 905.3 |
| 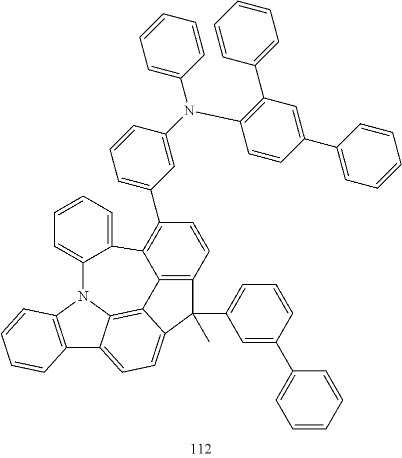 [SMILES: CC1(c2cccc(-c3ccccc3)c2)c2ccc(-c3cccc(N(c4ccccc4)c4ccc(-c5ccccc5)cc4-c4ccccc4)c3)c3c2-c2c1ccc1c4ccccc4n(c21)-c1ccccc1-3] | 65.4 | 891.4 |
| 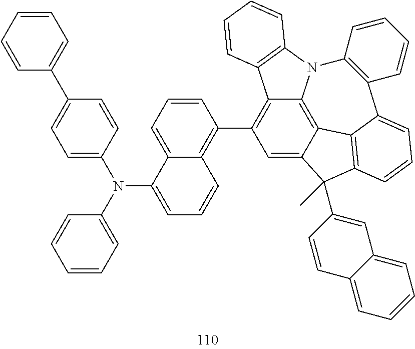 [SMILES: CC1(c2ccc3ccccc3c2)c2cccc3c2-c2c1cc(-c1cccc4c(N(c5ccccc5)c5ccc(-c6ccccc6)cc5)cccc14)c1c4ccccc4n(c21)-c1ccccc1-3] | 63.7 | 839.3 |
| 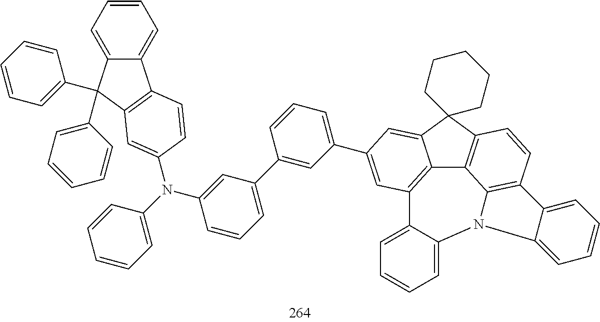 [SMILES: c1ccc(N(c2cccc(-c3cccc(-c4cc5c6c(c4)C4(CCCCC4)c4ccc7c8ccccc8n(c7c4-6)-c4ccccc4-5)c3)c2)c2ccc3c(c2)C(c2ccccc2)(c2ccccc2)c2ccccc2-3)cc1] | 66.1 | 957.4 |
| 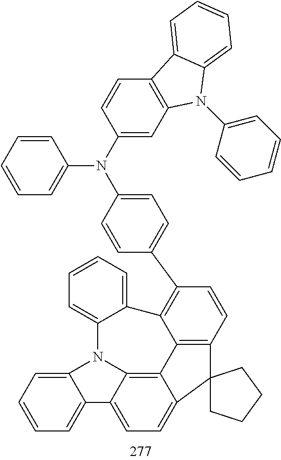 [SMILES: c1ccc(N(c2ccc(-c3ccc4c5c3-c3ccccc3-n3c6ccccc6c6ccc(c-5c63)C43CCCC3)cc2)c2ccc3c4ccccc4n(-c4ccccc4)c3c2)cc1] | 65.2 | 792.3 |
| 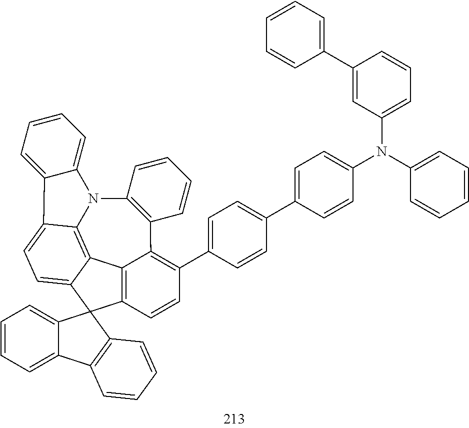 [SMILES: c1ccc(-c2cccc(N(c3ccccc3)c3ccc(-c4ccc(-c5ccc6c7c5-c5ccccc5-n5c8ccccc8c8ccc(c-7c85)C65c6ccccc6-c6ccccc65)cc4)cc3)c2)cc1] | 66.8 | 875.3 |
| 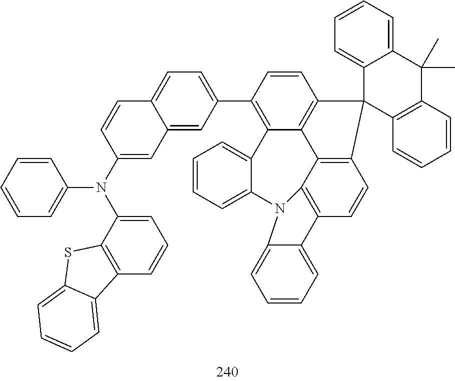 [SMILES: CC1(C)c2ccccc2C2(c3ccccc31)c1ccc(-c3ccc4ccc(N(c5ccccc5)c5cccc6c5sc5ccccc56)cc4c3)c3c1-c1c2ccc2c4ccccc4n(c12)-c1ccccc1-3] | 63.4 | 921.3 |

NMR data of some compounds were shown in Table 19 below.

TABLE 19

| Compound | NMR data |
|---|---|
| Compound 3 | ¹H NMR (400 Hz, CDCl₃) δ (ppm): 8.39-8.37 (d, 1H), |
| | 8.16-8.14 (d, 1H), 8.09-8.07 (d, 1H), 8.03-8.01 (d, 1H), |
| | 7.98-7.78 (m, 5H), 7.69-7.51 (m, 9H), 7.48-7.44 (t, 2H), |
| | 7.38-7.33 (t, 3H), 7.04-7.02 (d, 2H), 6.75 (d, 2H), 6.65- |
| | 6.61 (t, 1H), 6.54-6.52 (d, 2H), 1.87 (s, 6H). |
| Compound 25 | ¹H NMR (400 Hz, CDCl₃) δ (ppm): 8.18-8.15 (m, 2H), |
| | 8.11-8.09 (d, 1H), 8.0-7.33 (d, 1H), 7.91-7.89 (d, 1H), |
| | 7.87-7.84 (d, 1H), 7.82-7.77 (t, 1H), 7.69-7.65 (t, 1H), |
| | 7.56-7.41 (m, 9H), 7.39-7.24 (m, 5H), 7.17-7.09 (m, 2H), |
| | 6.99 (s, 1H), 6.90 (s, 1H), 6.80-6.78 (d, 1H), 6.48-6.46 |
| | (d, 1H), 1.8 (s, 6H). |

Fabrication and Performance Evaluation of OLEDs

### Example 1

Red Light-Emitting OLED

An ITO substrate with a thickness of 1,500 Å was cut into a size of 40 mm (length)×40 mm (width)×0.7 mm (thickness), then the substrate was processed through photolithography into an experimental substrate with cathode 200, anode 100, and insulating layer patterns, the experimental substrate was subjected to a surface treatment with UV-ozone and O₂:N₂ plasma to increase a work function of the anode 100 (experimental substrate), and surfaces of the ITO substrate were cleaned with an organic solvent to remove scums and oil stains on the surface of the ITO substrate.

A compound F4-TCNQ was vacuum-evaporated on the experimental substrate to form an HIL 310 with a thickness of 100 Å; and then a compound NPB was vacuum-evaporated on the HIL 310 to form an HTL 320 with a thickness of 950 Å.

The compound 1 was vacuum-evaporated on the HTL 320 to form an EBL 330 with a thickness of 850 Å.

Ir(piq)₂(acac) and CBP were co-evaporated on the EBL 330 in a film thickness ratio of 3%:97% to form an organic electroluminescent layer 340 with a thickness of 450 Å (red light-emitting layer, R-EML).

ET-06 and LiQ were mixed in a weight ratio of 1:1 and then deposited to form an ETL 350 with a thickness of 280 Å, and then Yb was evaporated on the ETL to form an EIL 360 with a thickness of 15 Å.

Magnesium (Mg) and silver (Ag) were vacuum-evaporated on the EIL in a film thickness ratio of 1:9 to form a cathode 200 with a thickness of 110 Å.

In addition, CP-5 was evaporated on the cathode 200 to form a capping layer (CPL) with a thickness of 630 Å, thereby completing the fabrication of the red light-emitting OLED.

The structural formulas of F4-TCNQ, NPB, Ir(piq)₂(acac), CBP, ET-06, LiQ, CP-5, compound A, compound B, and compound C were shown in Table 20 below.

TABLE 20

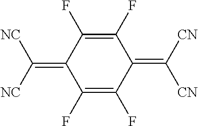 [SMILES: N#CC(C#N)=c1c(F)c(F)c(=C(C#N)C#N)c(F)c1F]

F4-TCNQ

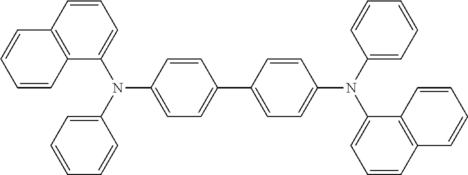 [SMILES: c1ccc(N(c2ccc(-c3ccc(N(c4ccccc4)c4cccc5ccccc45)cc3)cc2)c2cccc3ccccc23)cc1]

NPB

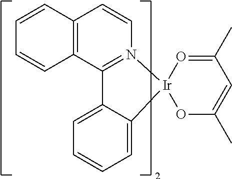

Ir(piq)₂(acac)

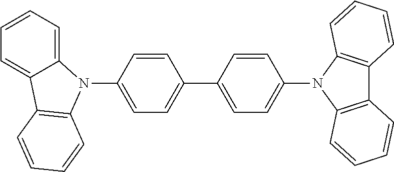 [SMILES: c1ccc2c(c1)c1ccccc1n2-c1ccc(-c2ccc(-n3c4ccccc4c4ccccc43)cc2)cc1]

CBP

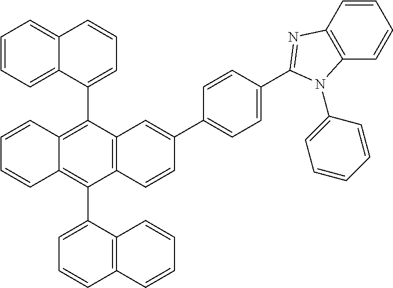 [SMILES: c1ccc(-n2c(-c3ccc(-c4ccc5c(-c6cccc7ccccc67)c6ccccc6c(-c6cccc7ccccc67)c5c4)cc3)nc3ccccc32)cc1]

ET-06

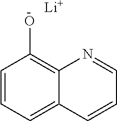 [SMILES: [Li+] ; Oc1cccc2cccnc12]

LiQ

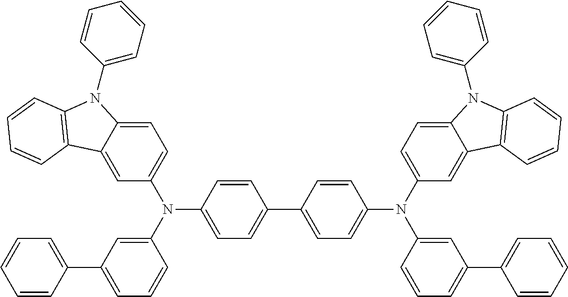 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

CP-5

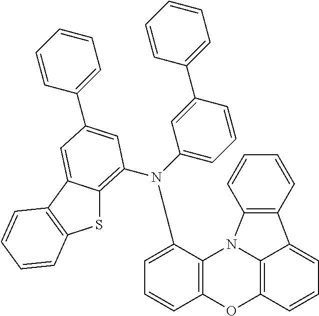 [SMILES: c1ccc(-c2cccc(N(c3cccc4c3-n3c5ccccc5c5cccc(c53)O4)c3cc(-c4ccccc4)cc4c3sc3ccccc34)c2)cc1]

Compound A

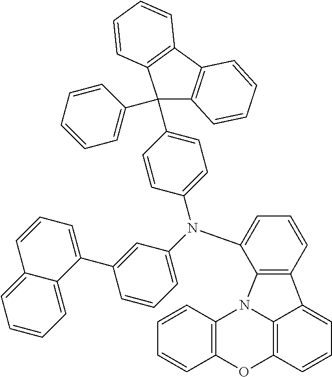 [SMILES: c1ccc(C2(c3ccc(N(c4cccc(-c5cccc6ccccc56)c4)c4cccc5c6cccc7c6n(c45)-c4ccccc4O7)cc3)c3ccccc3-c3ccccc32)cc1]

Compound B

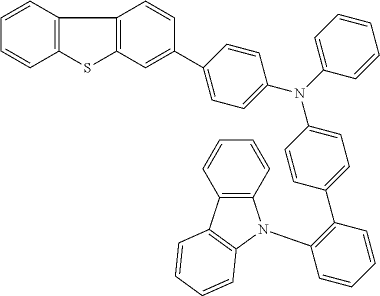 [SMILES: c1ccc(N(c2ccc(-c3ccc4c(c3)sc3ccccc34)cc2)c2ccc(-c3ccccc3-n3c4ccccc4c4ccccc43)cc2)cc1]

Compound C

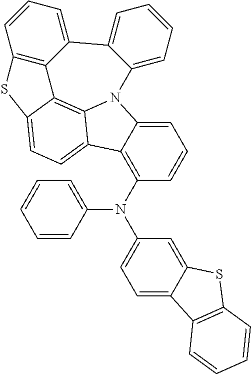 [SMILES: c1ccc(N(c2ccc3c(c2)sc2ccccc23)c2cccc3c2c2ccc4sc5cccc6c7ccccc7n3c2c4c56)cc1]

Compound D

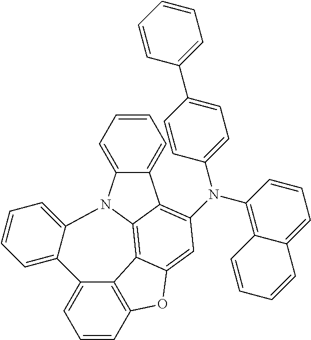 [SMILES: c1ccc(-c2ccc(N(c3cccc4ccccc34)c3cc4oc5cccc6c7ccccc7n7c8ccccc8c3c7c4c56)cc2)cc1]

Compound E

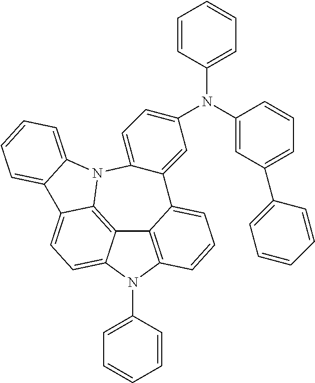 [SMILES: c1ccc(-c2cccc(N(c3ccccc3)c3ccc4c(c3)c3cccc5c3c3c(ccc6c7ccccc7n4c63)n5-c3ccccc3)c2)cc1]

Compound F

### Examples 2 to 50

Red light-emitting OLEDs were each preparated by the same method as in Example 1, except that the compounds listed in Table 21 were each used instead of the compound 1 in the formation of the EBL.

### Comparative Example 1

A red light-emitting OLED was preparated by the same method as in Example 1, except that a compound A was used instead of the compound 1 in the formation of the EBL.

### Comparative Example 2

A red light-emitting OLED was preparated by the same method as in Example 1, except that a compound B was used instead of the compound 1 in the formation of the EBL.

### Comparative Example 3

A red light-emitting OLED was preparated by the same method as in Example 1, except that a compound C was used instead of the compound 1 in the formation of the EBL.

### Comparative Example 4

A red light-emitting OLED was preparated by the same method as in Example 1, except that a compound D was used instead of the compound 1 in the formation of the EBL.

### Comparative Example 5

A red light-emitting OLED was preparated by the same method as in Example 1, except that a compound E was used instead of the compound 1 in the formation of the EBL.

### Comparative Example 6

A red light-emitting OLED was preparated by the same method as in Example 1, except that a compound F was used instead of the compound 1 in the formation of the EBL.

The OLEDs preparated above were subjected to performance analysis at 15 mA/cm², and results were shown in Table 21.

TABLE 21
Performance test results of the red light-emitting OLEDs

| | | | | | | External | |
|---|---|---|---|---|---|---|---|
| | | Driving | Current | Power | Chromaticity | quantum | T95 |
| | Compound | voltage | efficiency | efficiency | coordinate | efficiency | service life |
| Example | X | (V) | (cd/A) | (lm/W) | CIEx, CIEy | (EQE) (%) | (h) |
| Example 1 | Compound 1 | 3.20 | 40.29 | 32.89 | 0.680, 0.320 | 23.2 | 682 |
| Example 2 | Compound 3 | 3.28 | 40.70 | 33.18 | 0.680, 0.320 | 23.5 | 686 |
| Example 3 | Compound | 3.28 | 40.93 | 33.54 | 0.680, 0.320 | 23.0 | 683 |
| | 11 | | | | | | |
| Example 4 | Compound | 3.20 | 40.30 | 33.70 | 0.680, 0.320 | 23.3 | 680 |
| | 25 | | | | | | |
| Example 5 | Compound 4 | 3.19 | 40.27 | 33.92 | 0.680, 0.320 | 22.5 | 688 |
| Example 6 | Compound | 3.18 | 40.30 | 33.18 | 0.680, 0.320 | 21.8 | 685 |
| | 43 | | | | | | |
| Example 7 | Compound | 3.18 | 40.71 | 33.48 | 0.680, 0.320 | 23.6 | 687 |
| | 35 | | | | | | |
| Example 8 | Compound | 3.18 | 40.73 | 33.47 | 0.680, 0.320 | 22.8 | 680 |
| | 59 | | | | | | |
| Example 9 | Compound | 3.18 | 36.23 | 30.79 | 0.680, 0.320 | 23.2 | 684 |
| | 49 | | | | | | |
| Example 10 | Compound | 3.19 | 39.79 | 32.52 | 0.680, 0.320 | 22.2 | 685 |
| | 57 | | | | | | |
| Example 11 | Compound | 3.20 | 39.41 | 32.14 | 0.680, 0.320 | 21.9 | 686 |
| | 44 | | | | | | |
| Example 12 | Compound | 3.20 | 39.39 | 32.95 | 0.680, 0.320 | 22.6 | 685 |
| | 10 | | | | | | |
| Example 13 | Compound | 3.18 | 39.98 | 32.41 | 0.680, 0.320 | 23.7 | 685 |
| | 40 | | | | | | |
| Example 14 | Compound | 3.19 | 36.86 | 30.24 | 0.680, 0.320 | 23.7 | 680 |
| | 46 | | | | | | |
| Example 15 | Compound | 3.19 | 39.00 | 32.70 | 0.680, 0.320 | 22.3 | 687 |
| | 22 | | | | | | |
| Example 16 | Compound | 3.20 | 36.27 | 30.85 | 0.680, 0.320 | 22.5 | 684 |
| | 63 | | | | | | |
| Example 17 | Compound | 3.20 | 39.44 | 32.99 | 0.680, 0.320 | 22.6 | 687 |
| | 70 | | | | | | |
| Example 18 | Compound 8 | 3.20 | 39.92 | 32.39 | 0.680, 0.320 | 23.0 | 688 |
| Example 19 | Compound | 3.19 | 39.53 | 32.96 | 0.680, 0.320 | 23.4 | 688 |
| | 13 | | | | | | |
| Example 20 | Compound | 3.19 | 39.12 | 32.62 | 0.680, 0.320 | 23.1 | 684 |
| | 32 | | | | | | |
| Example 21 | Compound | 3.19 | 36.85 | 30.23 | 0.680, 0.320 | 23.7 | 682 |
| | 53 | | | | | | |
| Example 22 | Compound | 3.19 | 36.54 | 30.14 | 0.680, 0.320 | 22.7 | 685 |
| | 24 | | | | | | |
| Example 23 | Compound | 3.39 | 35.60 | 29.86 | 0.680, 0.320 | 22.0 | 623 |
| | 101 | | | | | | |
| Example 24 | Compound | 3.49 | 34.58 | 29.68 | 0.680, 0.320 | 22.7 | 595 |
| | 196 | | | | | | |
| Example 25 | Compound | 3.58 | 34.50 | 29.58 | 0.680, 0.320 | 22.7 | 599 |
| | 179 | | | | | | |
| Example 26 | Compound | 3.38 | 35.51 | 29.86 | 0.680, 0.320 | 22.0 | 629 |
| | 115 | | | | | | |
| Example 27 | Compound | 3.39 | 35.17 | 29.81 | 0.680, 0.320 | 23.5 | 602 |
| | 154 | | | | | | |
| Example 28 | Compound | 3.59 | 34.20 | 29.39 | 0.680, 0.320 | 23.2 | 597 |
| | 138 | | | | | | |
| Example 29 | Compound | 3.41 | 36.62 | 30.90 | 0.680, 0.320 | 23.5 | 628 |
| | 116 | | | | | | |
| Example 30 | Compound | 3.42 | 35.64 | 29.84 | 0.680, 0.320 | 23.5 | 603 |
| | 170 | | | | | | |
| Example 31 | Compound | 3.20 | 39.92 | 32.56 | 0.680, 0.320 | 22.3 | 687 |
| | 265 | | | | | | |
| Example 32 | Compound | 3.21 | 40.78 | 33.20 | 0.680, 0.320 | 22.9 | 689 |
| | 218 | | | | | | |
| Example 33 | Compound | 3.39 | 35.50 | 30.28 | 0.680, 0.320 | 22.0 | 627 |
| | 259 | | | | | | |
| Example 34 | Compound | 3.21 | 40.01 | 33.57 | 0.680, 0.320 | 22.3 | 686 |
| | 244 | | | | | | |
| Example 35 | Compound | 3.38 | 36.69 | 30.34 | 0.680, 0.320 | 22.8 | 620 |
| | 252 | | | | | | |
| Example 36 | Compound | 3.42 | 35.88 | 30.40 | 0.680, 0.320 | 22.2 | 604 |
| | 220 | | | | | | |
| Example 37 | Compound | 3.39 | 35.65 | 30.23 | 0.680, 0.320 | 22.8 | 601 |
| | 243 | | | | | | |
| Example 38 | Compound | 3.42 | 35.42 | 30.84 | 0.680, 0.320 | 22.6 | 602 |
| | 219 | | | | | | |
| Example 39 | Compound | 3.38 | 36.46 | 30.98 | 0.680, 0.320 | 23.4 | 622 |
| | 107 | | | | | | |
| Example 40 | Compound | 3.38 | 35.60 | 30.26 | 0.680, 0.320 | 22.8 | 601 |
| | 111 | | | | | | |
| Example 41 | Compound | 3.40 | 35.25 | 30.83 | 0.680, 0.320 | 22.5 | 602 |
| | 106 | | | | | | |
| Example 42 | Compound | 3.41 | 35.07 | 30.44 | 0.680, 0.320 | 23.1 | 602 |
| | 134 | | | | | | |
| Example 43 | Compound | 3.42 | 35.60 | 30.16 | 0.680, 0.320 | 22.0 | 603 |
| | 150 | | | | | | |
| Example 44 | Compound | 3.42 | 35.90 | 30.41 | 0.680, 0.320 | 22.2 | 604 |
| | 142 | | | | | | |
| Example 45 | Compound | 3.38 | 36.47 | 30.99 | 0.680, 0.320 | 23.4 | 593 |
| | 112 | | | | | | |
| Example 46 | Compound | 3.41 | 35.56 | 30.20 | 0.680, 0.320 | 22.0 | 595 |
| | 110 | | | | | | |
| Example 47 | Compound | 3.58 | 34.25 | 29.97 | 0.680, 0.320 | 22.5 | 593 |
| | 264 | | | | | | |
| Example 48 | Compound | 3.18 | 39.47 | 32.32 | 0.680, 0.320 | 21.9 | 683 |
| | 277 | | | | | | |
| Example 49 | Compound | 3.42 | 35.23 | 29.80 | 0.680, 0.320 | 22.5 | 593 |
| | 213 | | | | | | |
| Example 50 | Compound | 3.58 | 34.01 | 29.68 | 0.680, 0.320 | 23.0 | 596 |
| | 240 | | | | | | |
| Comparative | Compound | 3.94 | 26.43 | 26.87 | 0.680, 0.320 | 22.7 | 442 |
| Example 1 | A | | | | | | |
| Comparative | Compound | 3.80 | 26.29 | 26.69 | 0.680, 0.320 | 22.2 | 452 |
| Example 2 | B | | | | | | |
| Comparative | Compound | 3.78 | 25.70 | 26.18 | 0.680, 0.320 | 21.5 | 456 |
| Example 3 | C | | | | | | |
| Comparative | Compound | 3.78 | 30.73 | 27.54 | 0.680, 0.320 | 24.0 | 503 |
| Example 4 | D | | | | | | |
| Comparative | Compound | 3.80 | 30.50 | 27.70 | 0.680, 0.320 | 24.3 | 510 |
| Example 5 | E | | | | | | |
| Comparative | Compound | 3.99 | 28.27 | 26.82 | 0.680, 0.320 | 23.1 | 478 |
| Example 6 | F | | | | | | |

According to the results shown in Table 21, various performances of the OLEDs with the organic compound of the present disclosure as an EBL exhibited in Examples 1 to 50 are better than that of the OLEDs exhibited in the comparative examples. Compared with the OLEDs corresponding to the compounds in Comparative Examples 1 to 6 in the prior art, the OLEDs with the organic compound of the present disclosure as an EBL preparated in Examples 1 to 50 have a driving voltage reduced by at least 0.19 V, a current efficiency (Cd/A) increased by at least 10.67%, and a service life increased by at least 16.27% (the service life can be increased by up to 179 h). It can be seen from the above data that, when the organic compound of the present disclosure is used as an EBL of an electronic device, both the light-emitting efficiency (Cd/A) and the service life (T95) of the electronic device are significantly improved.

The above variations and modifications fall within the scope of the present disclosure. It should be understood that the present disclosure disclosed and defined in this specification can extend to all alternative combinations of two or more individual features mentioned or apparent in the text and/or accompanying drawings. All of these different combinations constitute various alternative aspects of the present disclosure. The implementations described in this specification illustrate the known optimal manner for implementing the present disclosure, and enable those skilled in the art to use the present disclosure.

Those of ordinary skill in the art can understand that the above implementations are specific embodiments for implementing the present disclosure; and in practical applications, various changes may be made in terms of forms and details without departing from the spirit and scope of the present disclosure.

1. An organic compound with a general structure shown in chemical formula 1: 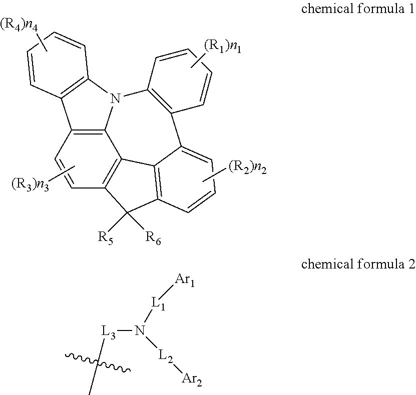
wherein R₅ and R₆ are the same or different, and are each independently selected from the group consisting of alkyl with 1 to 10 carbon atoms, aryl with 6 to 20 carbon atoms, haloalkyl with 1 to 10 carbon atoms, cycloalkyl with 3 to 10 carbon atoms, heteroaryl with 3 to 20 carbon atoms, hydrogen, deuterium, halogen, and cyano; or R₅ and R₆ are optionally connected to form a substituted or unsubstituted 5- to 18-membered aliphatic ring or 5- to 18-membered aromatic ring together with the carbon atom to which they are jointly connected, and a substituent in the 5-to 18-membered aliphatic ring or 5- to 18-membered aromatic ring is independently selected from the group consisting of deuterium, halogen, cyano, alkyl with 1 to 10 carbon atoms, haloalkyl with 1 to 10 carbon atoms, and deuterated alkyl with 1 to 10 carbon atoms;
R₁, R₂, R₃, and R₄ are the same or different, and are each independently selected from the group consisting of hydrogen, deuterium, halogen, cyano, alkyl with 1 to 10 carbon atoms, cycloalkyl with 3 to 10 carbon atoms, aryl with 6 to 20 carbon atoms, heteroaryl with 3 to 12 carbon atoms, and a group shown in chemical formula 2; and one, two, three, or four of R₁, R₂, R₃, and R₄ are the group shown in chemical formula 2;
R₁, R₂, R₃, and R₄ are collectively represented by Rᵢ, and n₁ to n₄ are collectively represented by nᵢ; nᵢ indicates a number of and i is a variable of 1, 2, 3, or 4; when i is 1 or 4, nᵢ is selected from the group consisting of 1, 2, 3, and 4; when i is 2, nᵢ is selected from the group consisting of 1, 2, and 3; when i is 3, nᵢ is selected from the group consisting of 1 and 2; and when nᵢ is greater than 1, any two nᵢ values are the same or different;
L₁, L₂, and L₃ are the same or different, and are each independently selected from the group consisting of a single bond, substituted or unsubstituted arylene with 6 to 30 carbon atoms, and substituted or unsubstituted heteroarylene with 3 to 30 carbon atoms;
Ar₁ and Ar₂ are the same or different, and are each independently selected from the group consisting of substituted or unsubstituted aryl with 6 to 30 carbon atoms and substituted or unsubstituted heteroaryl with 3 to 30 carbon atoms;
substituents in L₁, L₂, L₃, Ar₁, and Ar₂ are the same or different, and are each independently selected from the group consisting of deuterium, halogen, cyano, heteroaryl with 3 to 20 carbon atoms, aryl with 6 to 20 carbon atoms, alkyl with 1 to 10 carbon atoms, haloalkyl with 1 to 10 carbon atoms, deuterated alkyl with 1 to 10 carbon atoms, alkoxy with 1 to 10 carbon atoms, cycloalkyl with 3 to 10 carbon atoms, alkylthio with 1 to 10 carbon atoms, trialkylsilyl with 3 to 12 carbon atoms, arylsilyl with 6 to 18 carbon atoms, aryloxy with 6 to 20 carbon atoms, and arylthio with 6 to 20 carbon atoms; or any two adjacent substituents in L₁, L₂, L₃, An, and Ar₂ are optionally connected to form a 5- to 13-membered aliphatic ring or a 5- to 13-membered aromatic ring.

2. The organic compound according to claim 1, wherein L₁, L₂, and L₃ are the same or different, and are each independently selected from the group consisting of a single bond, substituted or unsubstituted arylene with 6 to 18 carbon atoms, and substituted or unsubstituted heteroarylene with 5 to 18 carbon atoms; and
optionally, substituents in L₁, L₂, and L₃ are the same or different, and are each independently selected from the group consisting of deuterium, halogen, cyano, phenyl, trialkylsilyl with 3 to 8 carbon atoms, alkyl with 1 to 4 carbon atoms, haloalkyl with 1 to 4 carbon atoms, deuterated alkyl with 1 to 4 carbon atoms, alkoxy with 1 to 4 carbon atoms, alkylthio with 1 to 4 carbon atoms, phenyl, naphthyl, biphenyl, anthracenyl, phenanthryl, pyridyl, dibenzothienyl, dibenzofuranyl, and carbazolyl.

3. The organic compound according to claim 1, wherein L₁, L₂, and L₃ are each independently selected from the group consisting of a single bond, substituted or unsubstituted phenylene, substituted or unsubstituted naphthylene, substituted or unsubstituted biphenylene, substituted or unsubstituted anthracenylene, substituted or unsubstituted phenanthrylene, substituted or unsubstituted dibenzofuranylene, substituted or unsubstituted dibenzothienylene, substituted or unsubstituted fluorenylene, substituted or unsubstituted carbazolylene, and a group obtained by linking two or three of the above groups through a single bond; and
optionally, substituents in L₁, L₂, and L₃ are each independently selected from the group consisting of deuterium, fluorine, cyano, methyl, ethyl, n-propyl, isopropyl, tert-butyl, trifluoromethyl, trideuteromethyl, trimethylsilyl, cyclopentyl, phenyl, biphenyl, naphthyl, anthracenyl, phenanthryl, dibenzothienyl, dibenzofuranyl, carbazolyl, and pyridyl.

4. The organic compound according to claim 1, wherein L₁, L₂, and L₃ are each independently selected from the group consisting of a single bond and a substituted or unsubstituted group V; an unsubstituted group V is selected from the group consisting of the following groups: 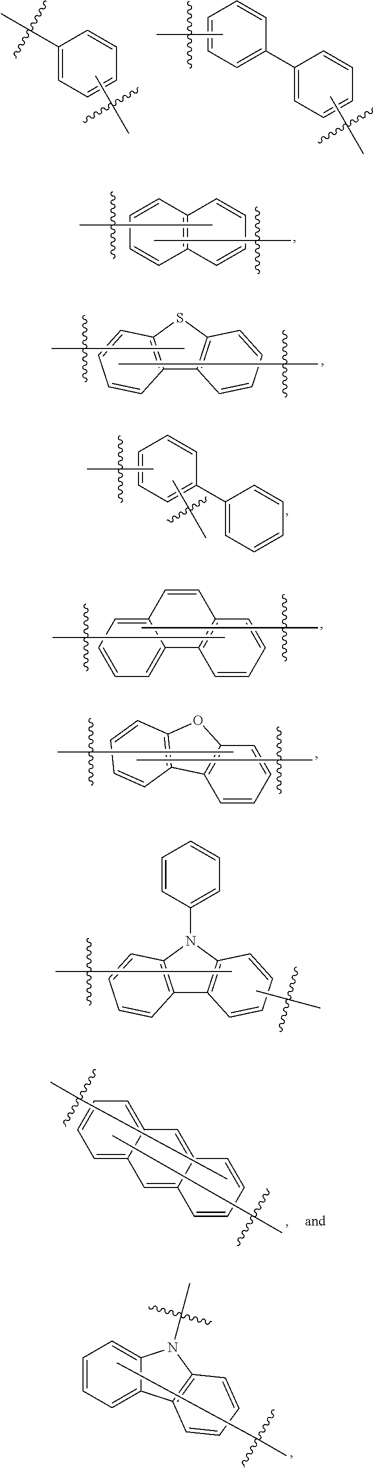
wherein [symbol] represents a chemical bond; a substituted group V has one or more substituents, and the one or more substituents are each independently selected from the group consisting of deuterium, cyano, fluorine, methyl, ethyl, n-propyl, isopropyl, tert-butyl, trifluoromethyl, trideuteromethyl, trimethylsilyl, phenyl, naphthyl, and pyridyl; and when the group V has two or more substituents, the two or more substituents are the same or different.

5. The organic compound according to claim 1, wherein L₁, L₂, and L₃ are each independently selected from the group consisting of a single bond and the following groups:

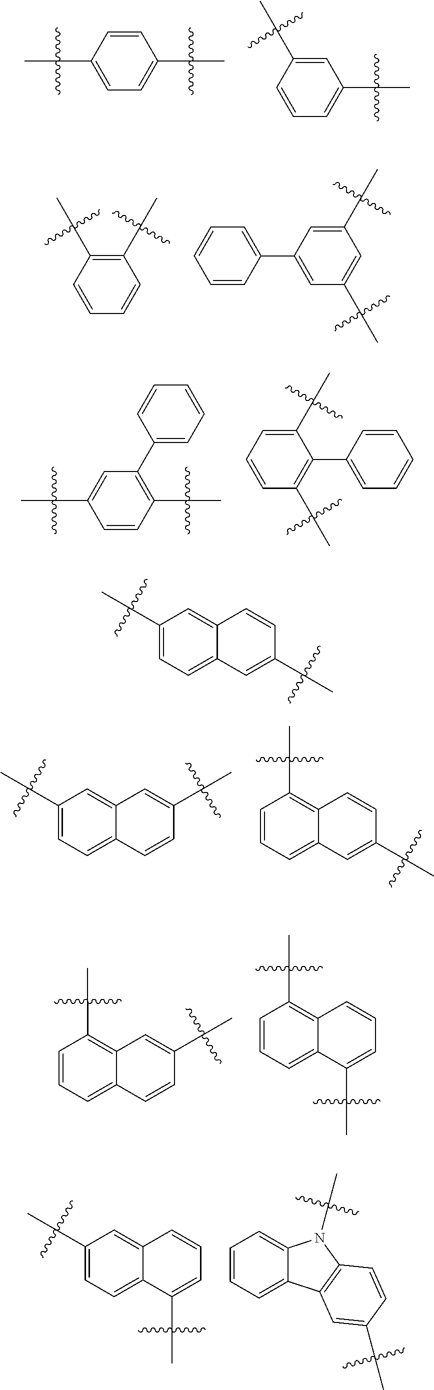

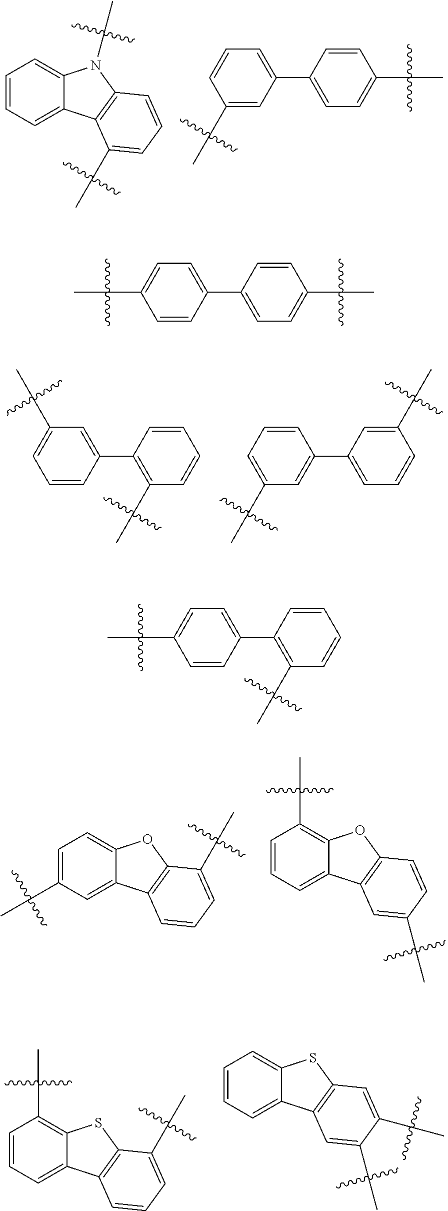

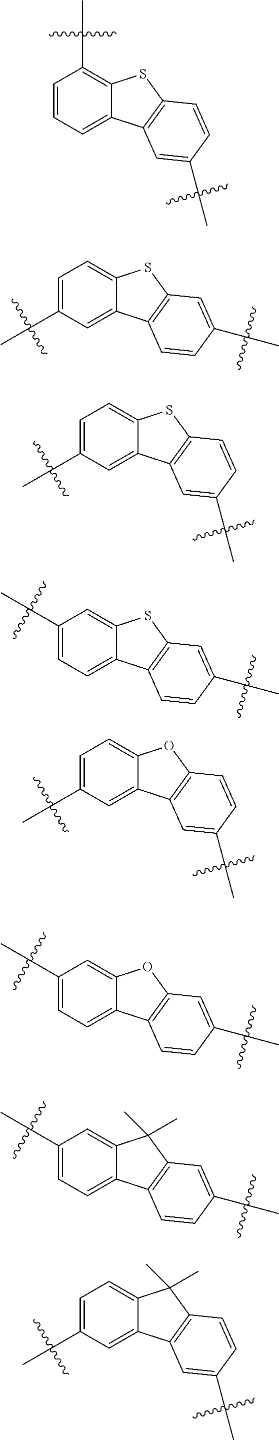

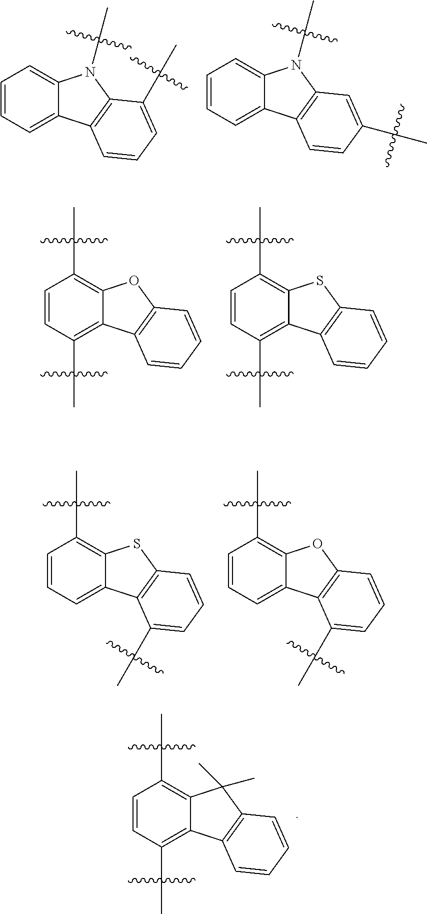

6. The organic compound according to claim 1, wherein Ar₁ and Ar₂ are the same or different, and are each independently selected from the group consisting of substituted or unsubstituted aryl with 6 to 25 carbon atoms and substituted or unsubstituted heteroaryl with 5 to 25 carbon atoms; and
optionally, substituents in Ar₁ and Ar₂ are each independently selected from the group consisting of deuterium, halogen, cyano, aryl with 6 to 15 carbon atoms, heteroaryl with 5 to 12 carbon atoms, alkyl with 1 to 4 carbon atoms, trialkylsilyl with 3 to 8 carbon atoms, cycloalkyl with 5 to 10 carbon atoms, haloalkyl with 1 to 4 carbon atoms, deuterated alkyl with 1 to 4 carbon atoms, alkoxy with 1 to 4 carbon atoms, and alkylthio with 1 to 4 carbon atoms.

7. The organic compound according to claim 1, wherein Ar₁ and Ar₂ are each independently selected from the group consisting of substituted or unsubstituted phenyl, substituted or unsubstituted biphenyl, substituted or unsubstituted terphenyl, substituted or unsubstituted naphthyl, substituted or unsubstituted phenanthryl, substituted or unsubstituted anthracenyl, substituted or unsubstituted dibenzofuranyl, substituted or unsubstituted dibenzothienyl, substituted or unsubstituted fluorenyl, substituted or unsubstituted carbazolyl, substituted or unsubstituted pyridyl, substituted or unsubstituted spirobifluorenyl, substituted or unsubstituted phenothiazinyl, and substituted or unsubstituted phenoxthiyl; and
optionally, substituents in Ar₁ and Ar₂ are each independently selected from the group consisting of deuterium, cyano, fluorine, methyl, ethyl, n-propyl, isopropyl, tert-butyl, trifluoromethyl, trideuteromethyl, trimethylsilyl, methoxy, isopropoxy, phenyl, cyclohexyl, phenyl, naphthyl, fluorenyl, dibenzothienyl, dibenzofuranyl, phenanthryl, and carbazolyl.

8. The organic compound according to claim 1, wherein Ar₁ and Ar₂ are each independently a substituted or unsubstituted group W; an unsubstituted group W is selected from the group consisting of the following groups: 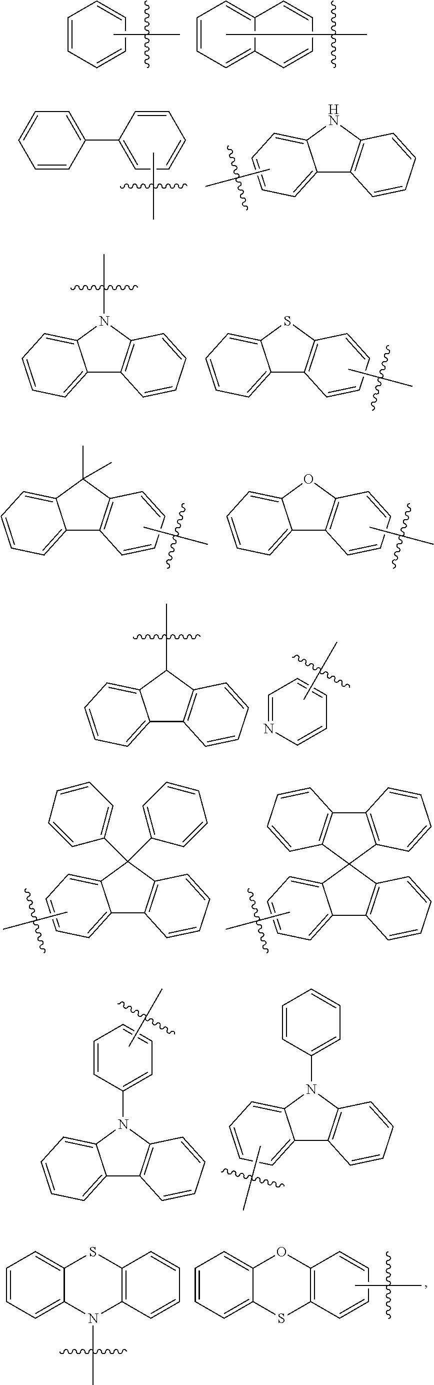
wherein a 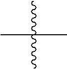
represents a chemical bond; a substituted group W has one or more substituents, and the one or more substituents are each independently selected from the group consisting of deuterium, cyano, fluorine, methyl, ethyl, n-propyl, isopropyl, tert-butyl, trifluoromethyl, trideuteromethyl, trimethylsilyl, methoxy, isopropoxy, phenyl, cyclohexyl, phenyl, naphthyl, fluorenyl, dibenzothienyl, dibenzofuranyl, phenanthryl, and carbazolyl; and when the group W has two or more substituents, the two or more substituents are the same or different.

9. The organic compound according to claim 1, wherein Ar₁ and Ar₂ are each independently selected from the group consisting of the following groups:

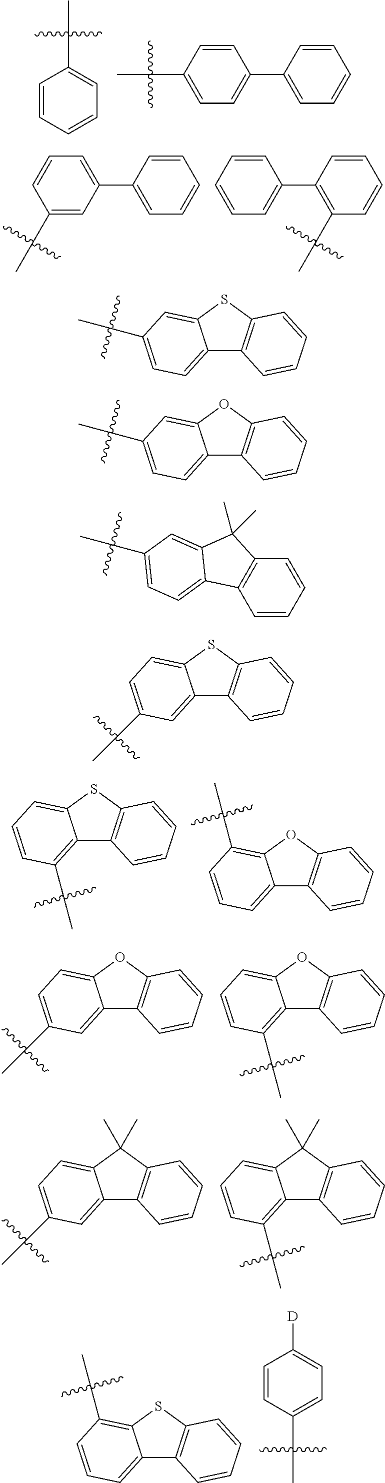

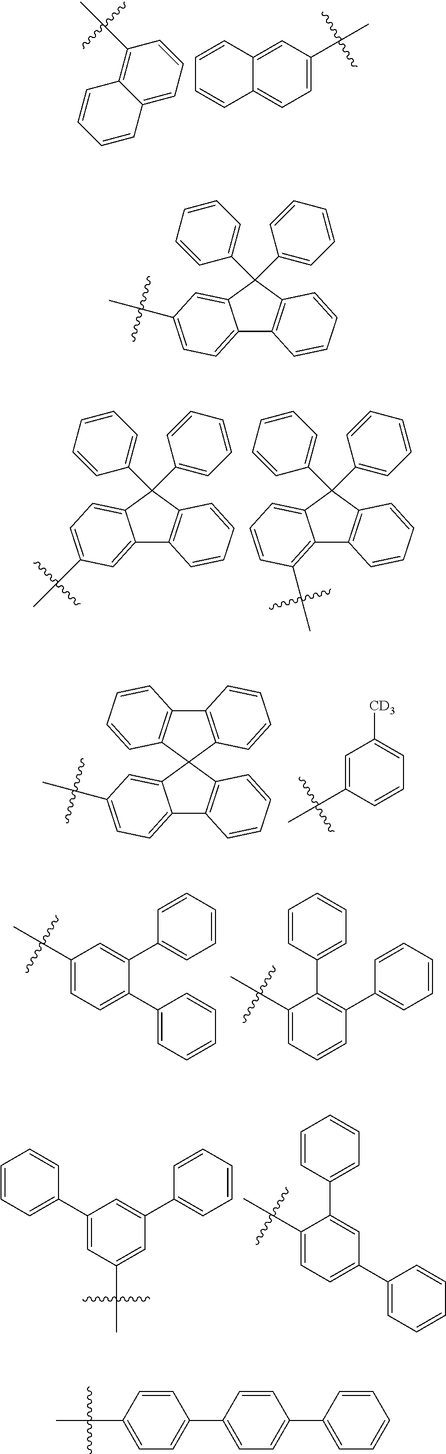

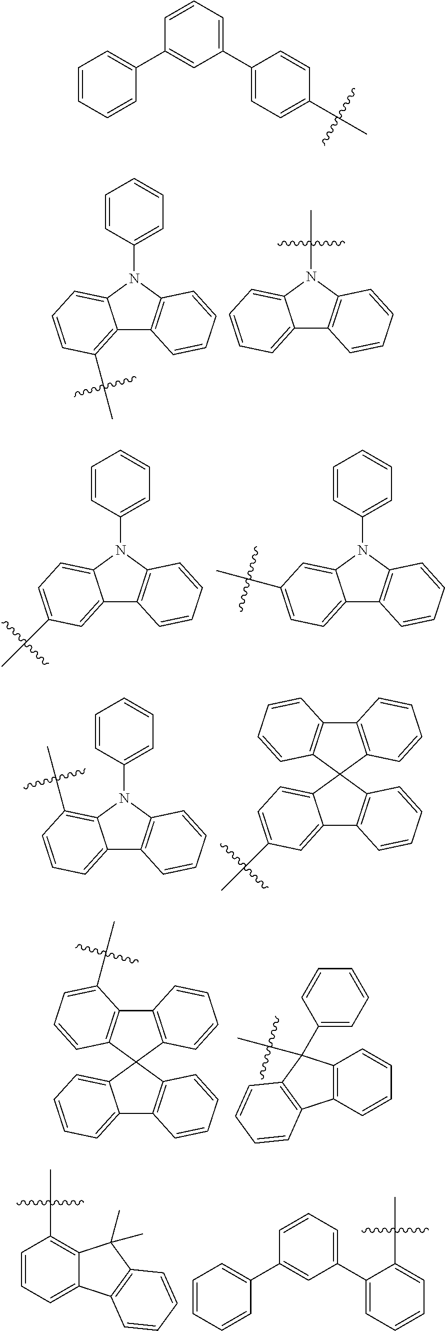

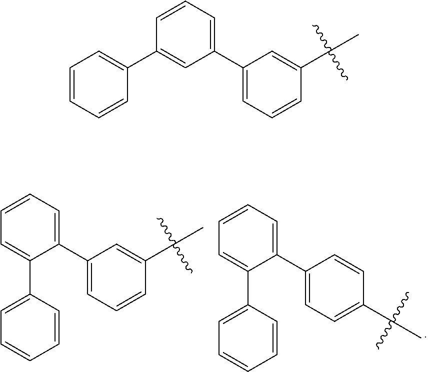

10. The organic compound according to claim 1, wherein R₁, R₂, R₃, and R₄ are each independently selected from the group consisting of hydrogen, deuterium, cyano, fluorine, methyl, ethyl, isopropyl, tert-butyl, trifluoromethyl, trideuteromethyl, trimethylsilyl, phenyl, naphthyl, biphenyl, dimethylfluorenyl, pyridyl, pyrimidinyl, quinolinyl, isoquinolinyl, carbazolyl, dibenzofuranyl, dibenzothienyl, dimethylfluorenyl, N-phenylcarbazolyl, and a group shown in chemical formula 2, and only one of R₁, R₂, R₃, and R₄ is the group shown in chemical formula 2.

11. The organic compound according to claim 1, wherein R₅ and R₆ are each independently selected from the group consisting of methyl, ethyl, n-propyl, isopropyl, tert-butyl, trifluoromethyl, trideuteromethyl, phenyl, naphthyl, biphenyl, terphenyl, fluorenyl, dimethylfluorenyl, anthracenyl, phenanthryl, pyridyl, dibenzothienyl, dibenzofuranyl, and carbazolyl; or R₅ and R₆ are optionally connected to form a fluorene ring, cyclopentane, cyclohexane, or 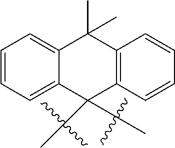 [SMILES: *C1(*)c2ccccc2C(C)(C)c2ccccc21]
together with the carbon atom to which they are jointly connected.

12. The organic compound according to claim 1, wherein R₅ and R₆ are each independently selected from the group consisting of methyl and the following groups: 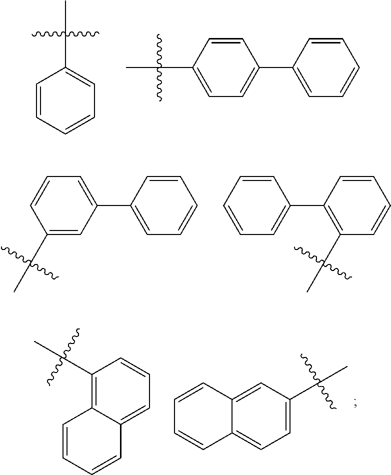
or R₅ and R₆ are optionally connected to form the following spiro-ring together with the carbon atom to which they are jointly connected:

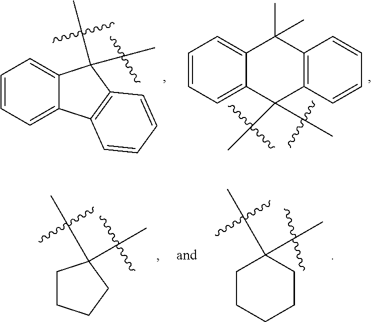

13. The organic compound according to claim 1, wherein the organic compound is selected from the group consisting of the following compounds:

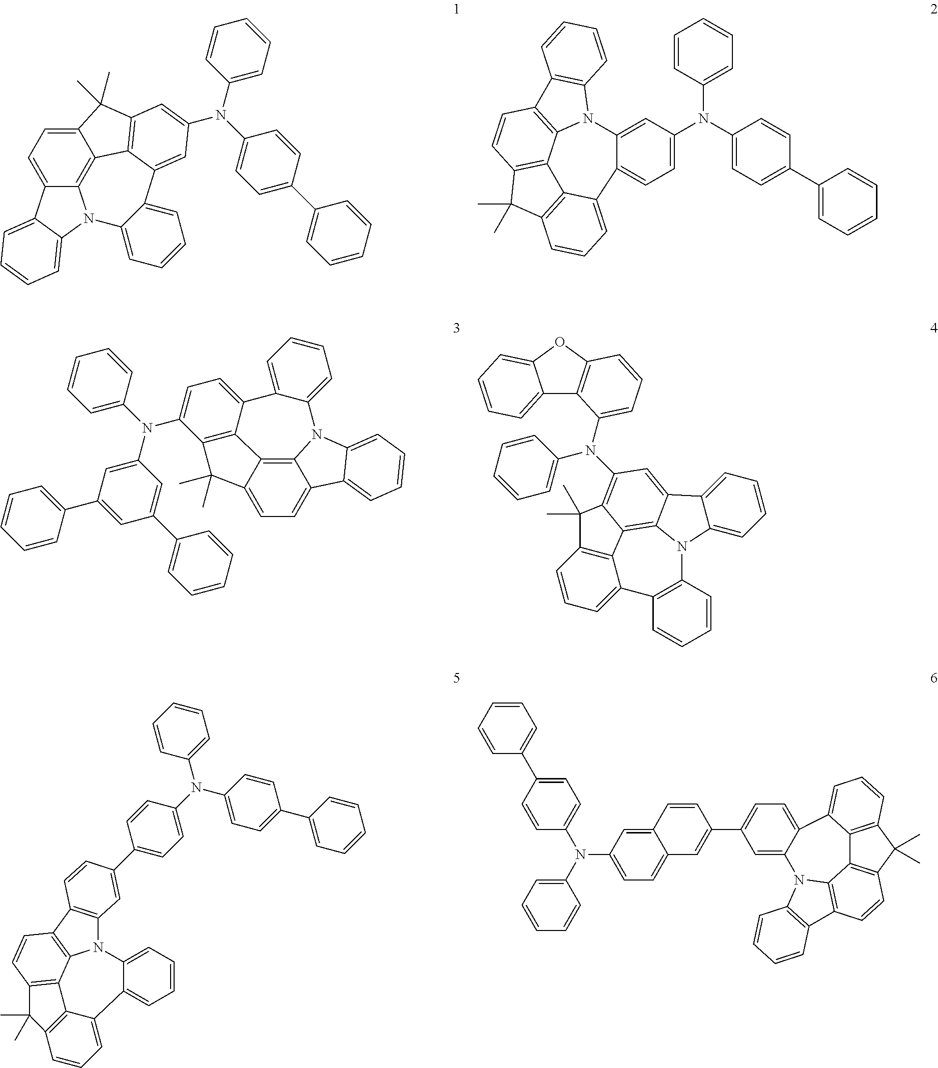

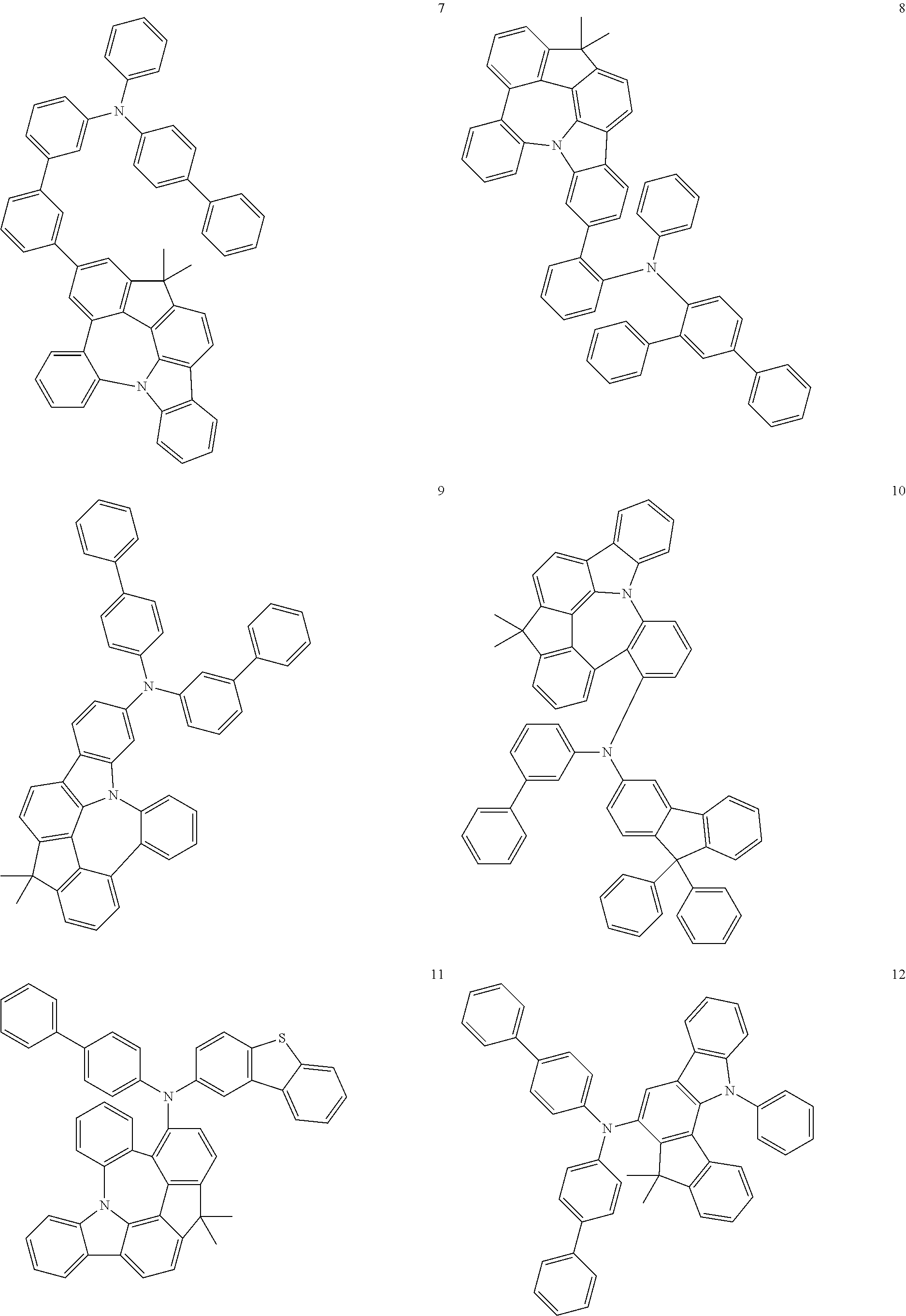

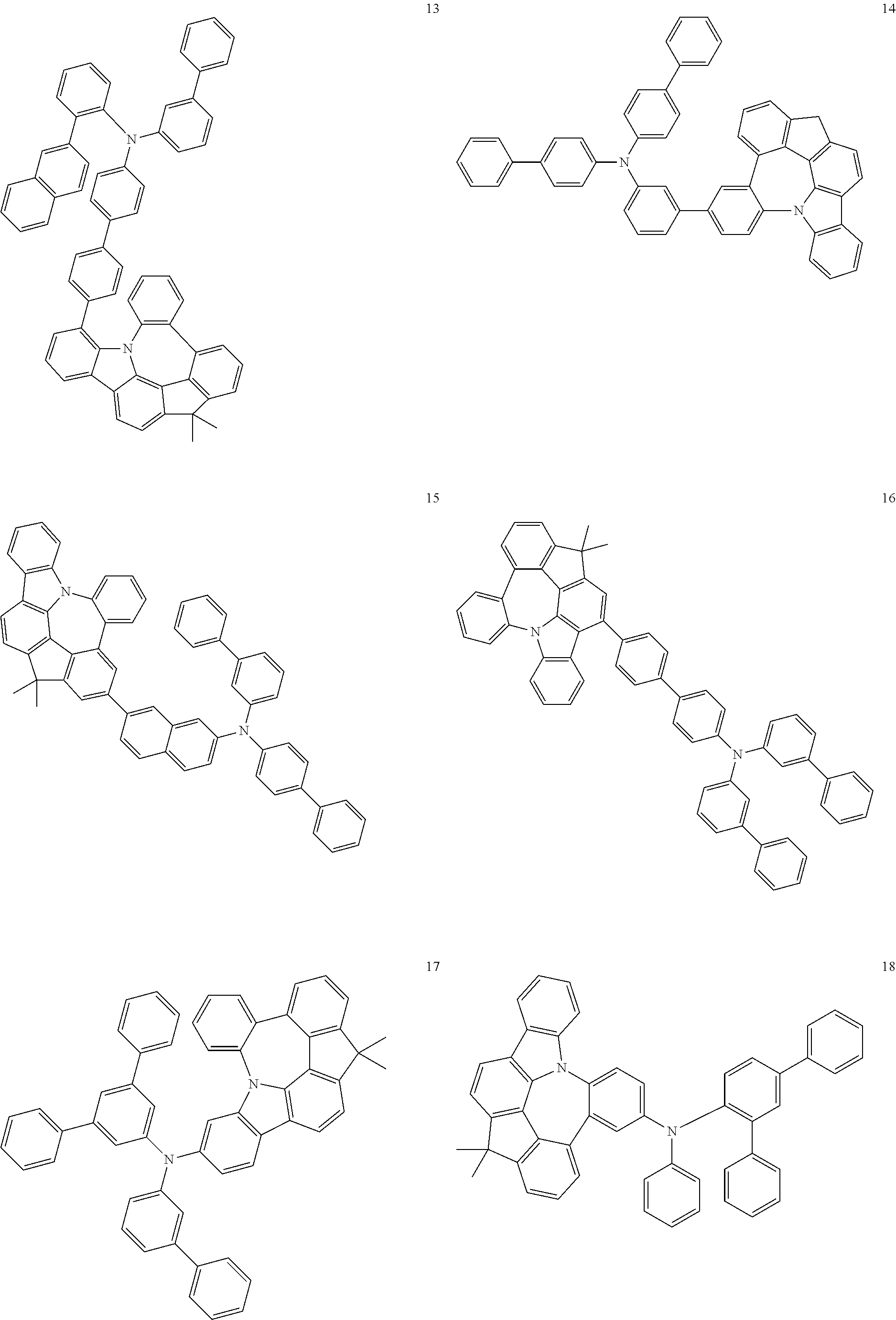

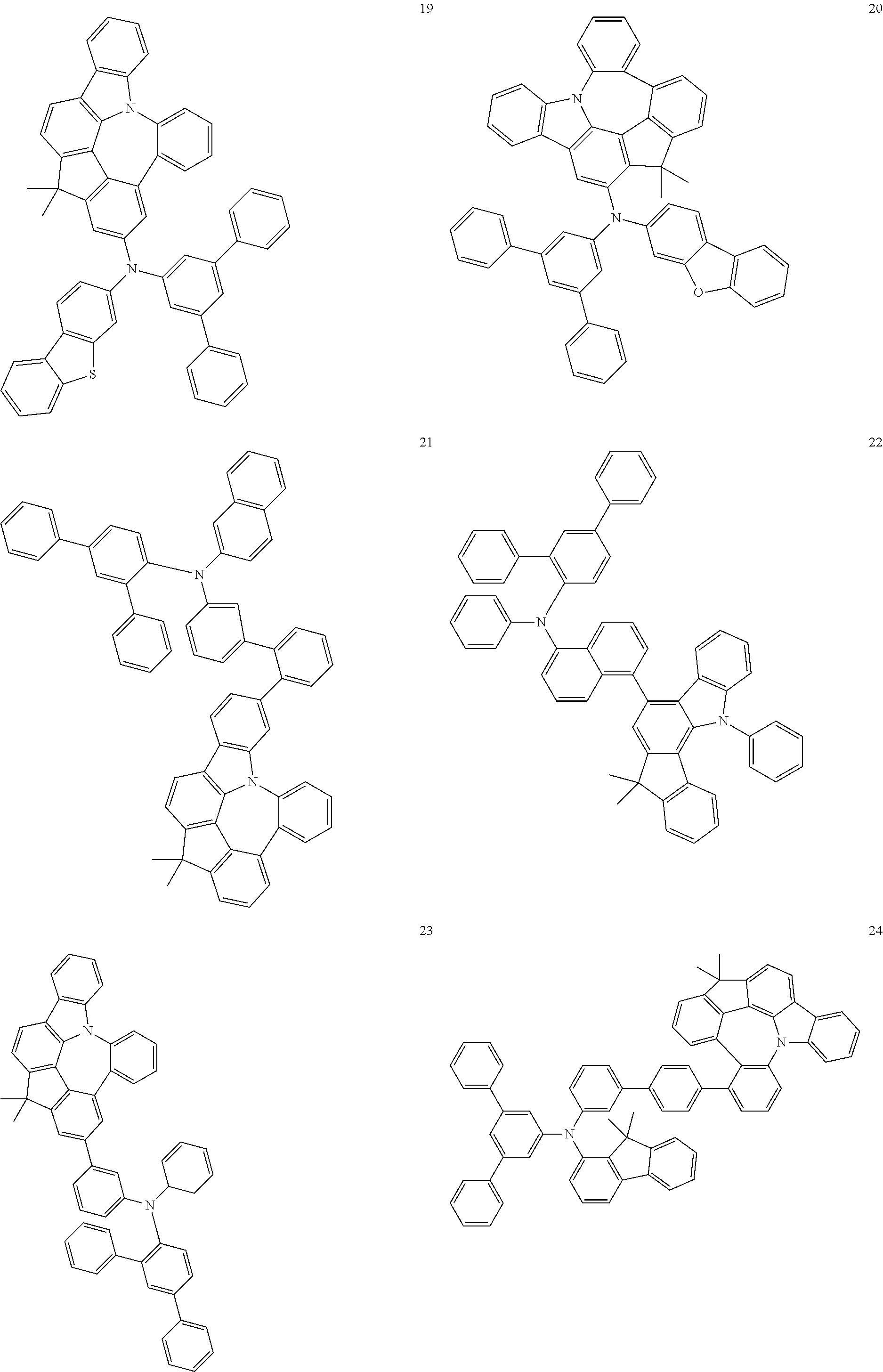

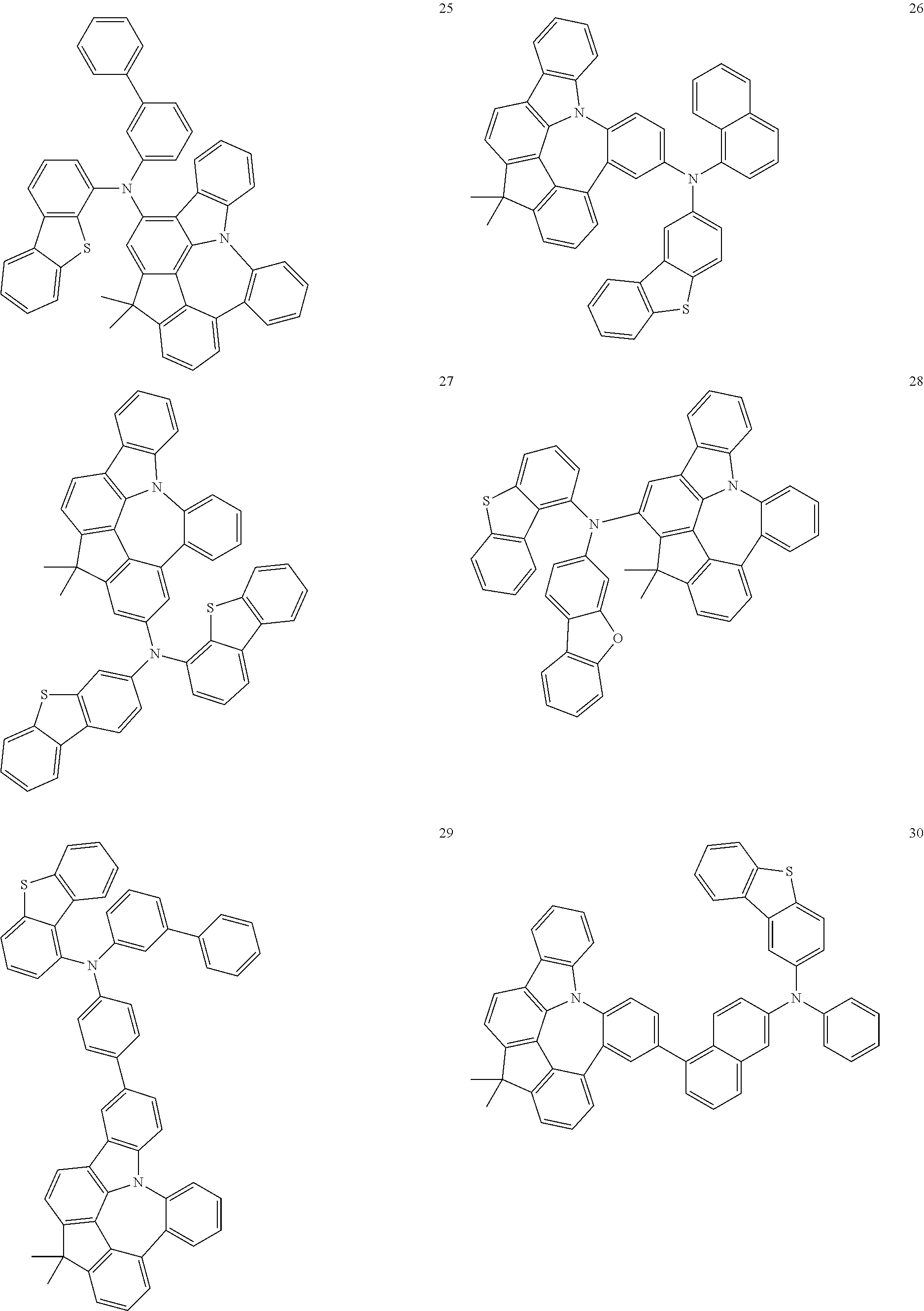

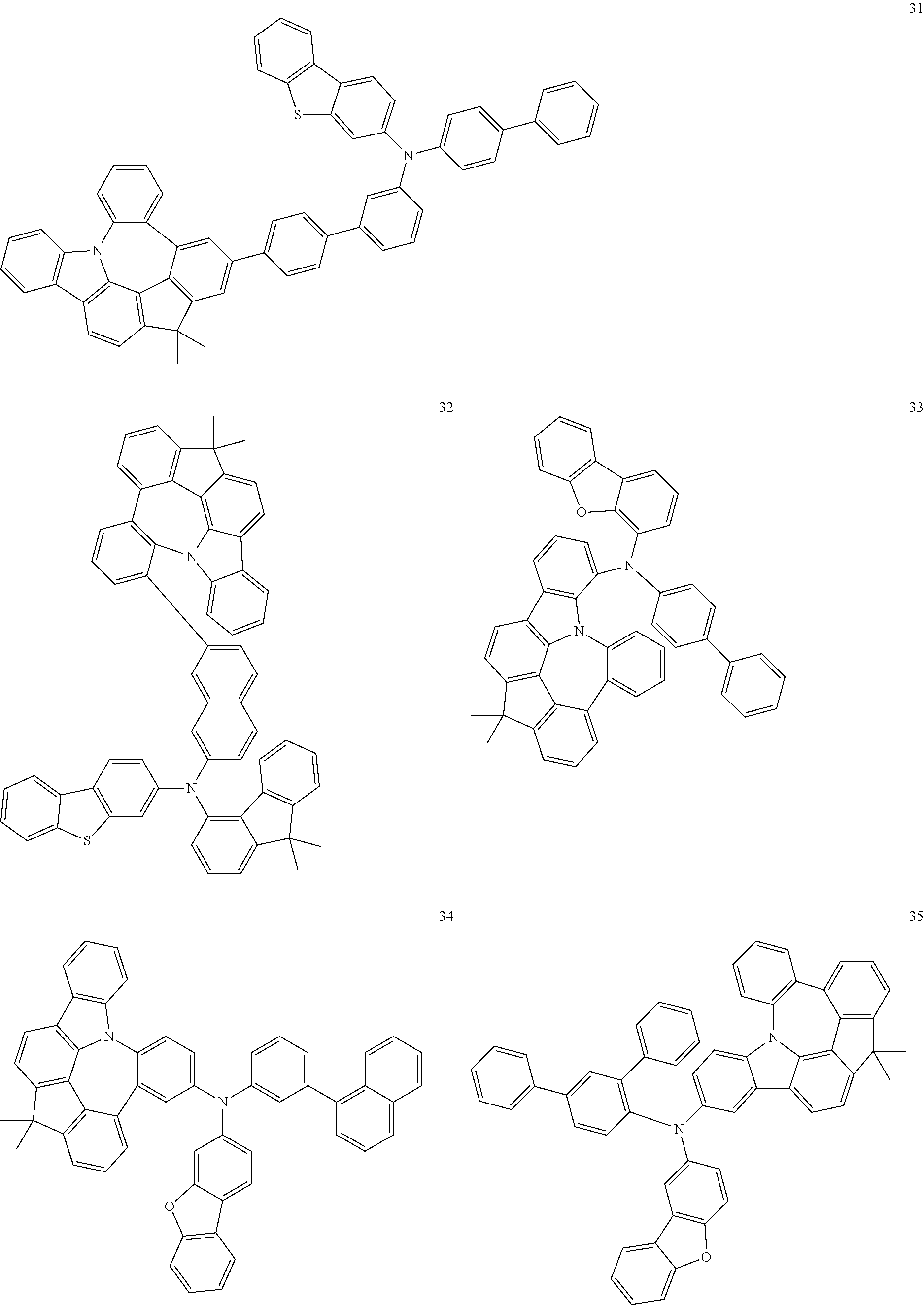

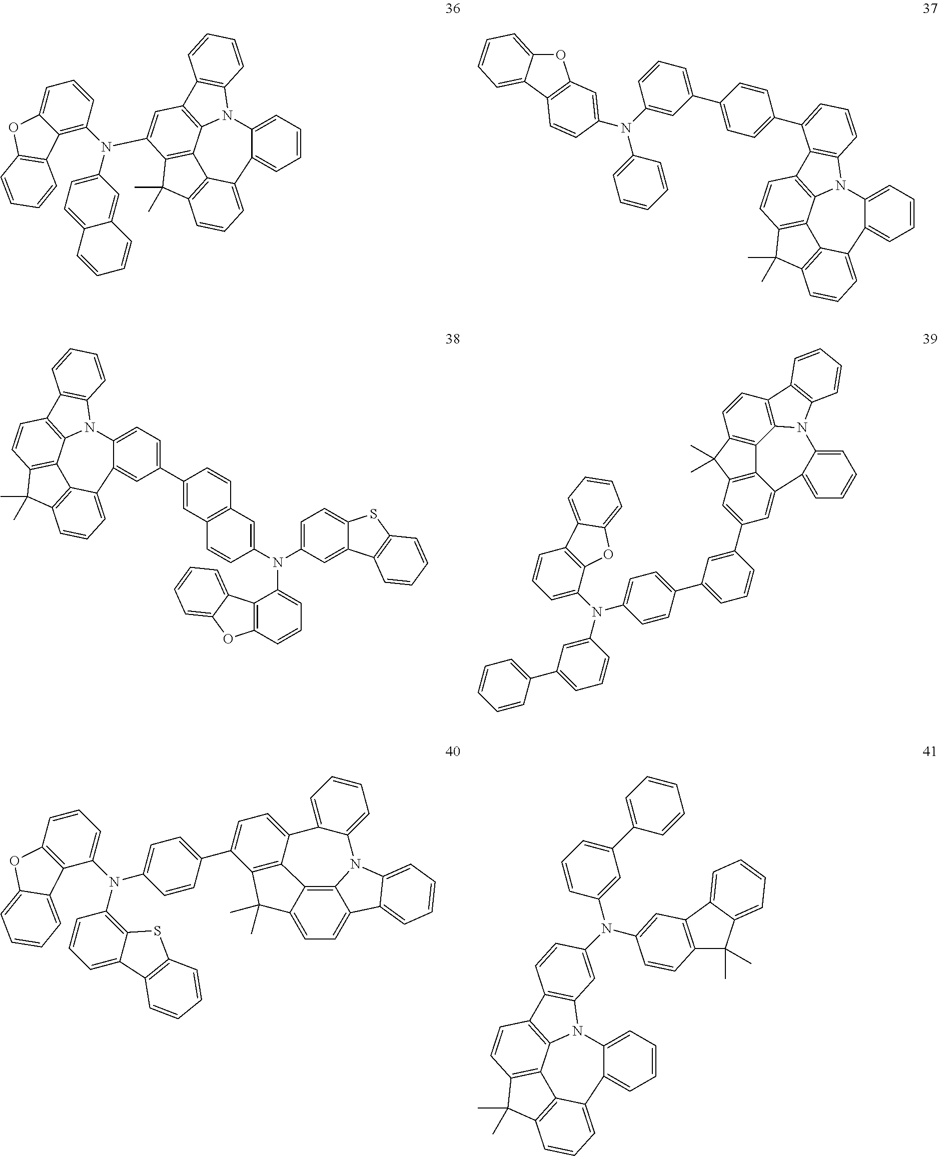

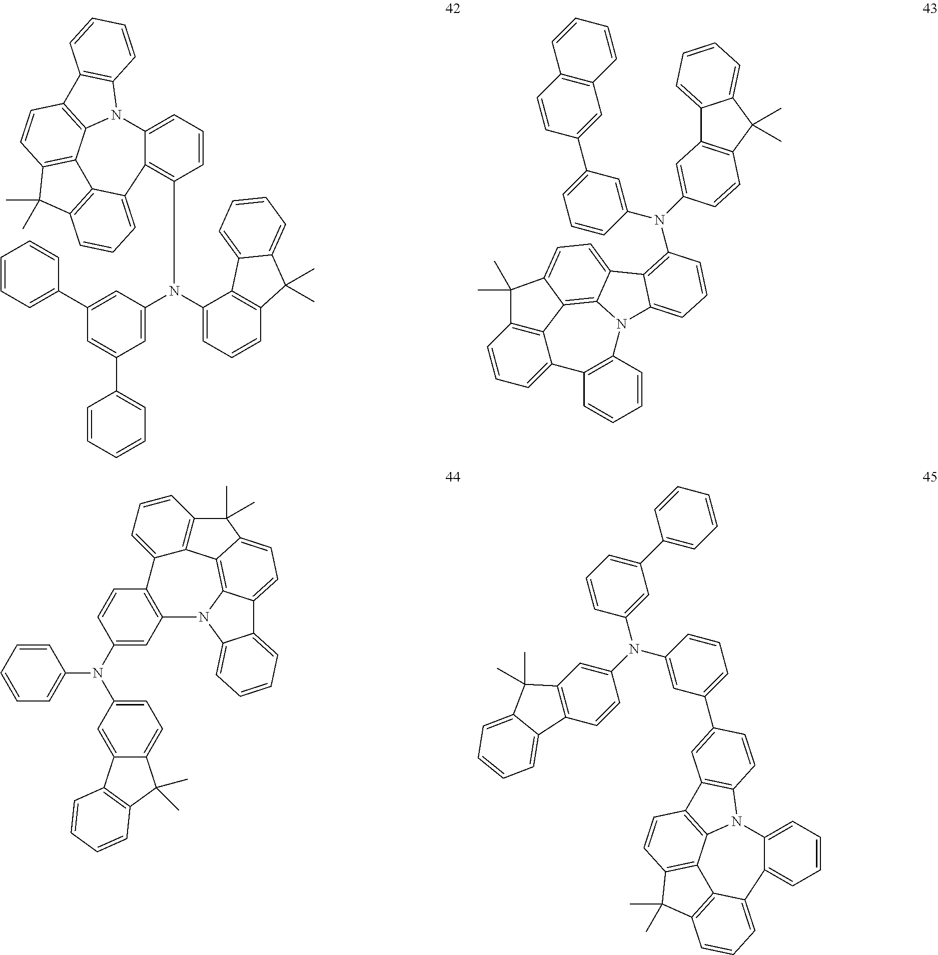

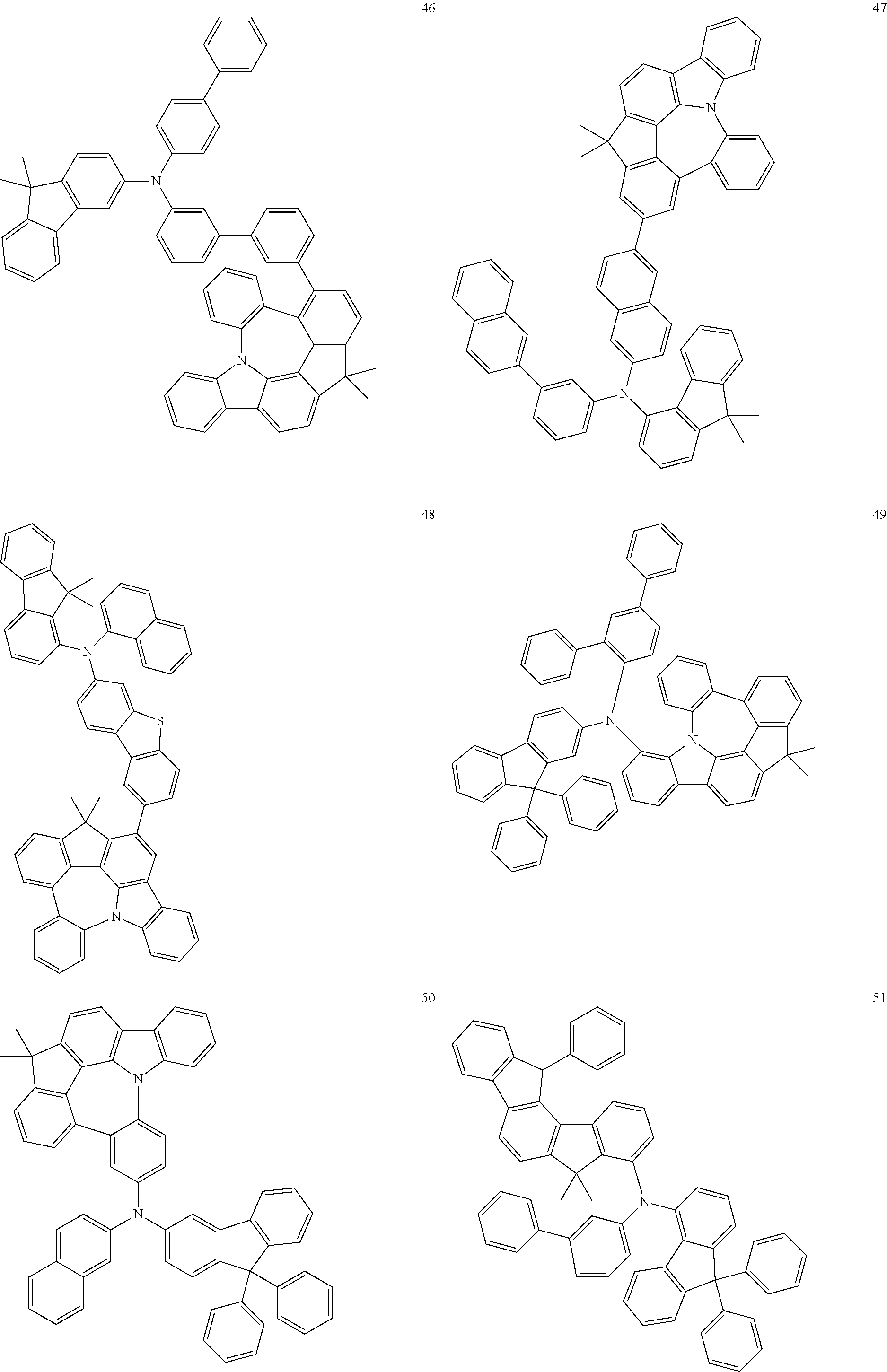

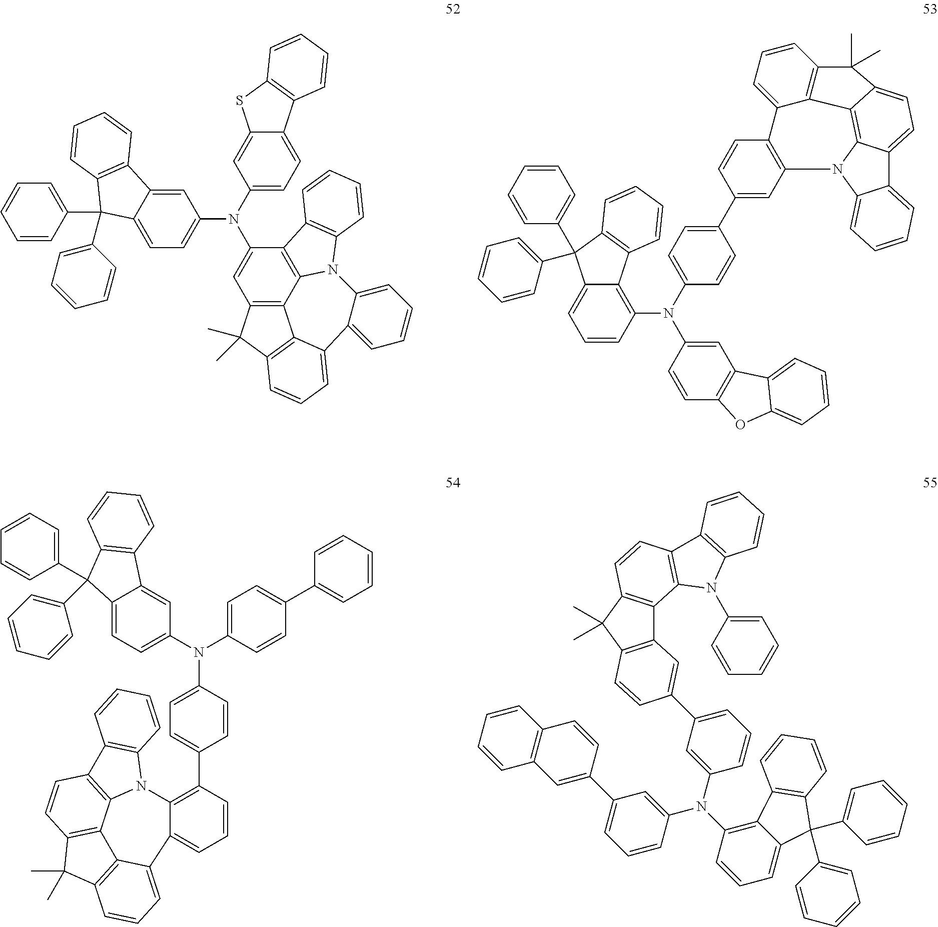 [SMILES: CC1(C)c2cccc3c2-c2c1cc(N(c1ccc4c(c1)-c1ccccc1C4(c1ccccc1)c1ccccc1)c1ccc4c(c1)sc1ccccc14)c1c4ccccc4n(c21)-c1ccccc1-3 ; CC1(C)c2cccc3c2-c2c1ccc1c4ccccc4n(c21)-c1cc(-c2ccc(N(c4ccc5oc6ccccc6c5c4)c4cccc5c4-c4ccccc4C5(c4ccccc4)c4ccccc4)cc2)ccc1-3 ; CC1(C)c2cccc3c2-c2c1ccc1c4ccccc4n(c21)-c1c(-c2ccc(N(c4ccc(-c5ccccc5)cc4)c4ccc5c(c4)-c4ccccc4C5(c4ccccc4)c4ccccc4)cc2)cccc1-3 ; CC1(C)c2ccc(-c3cccc(N(c4cccc(-c5ccc6ccccc6c5)c4)c4cccc5c4-c4ccccc4C5(c4ccccc4)c4ccccc4)c3)cc2-c2c1ccc1c3ccccc3n(-c3ccccc3)c21]

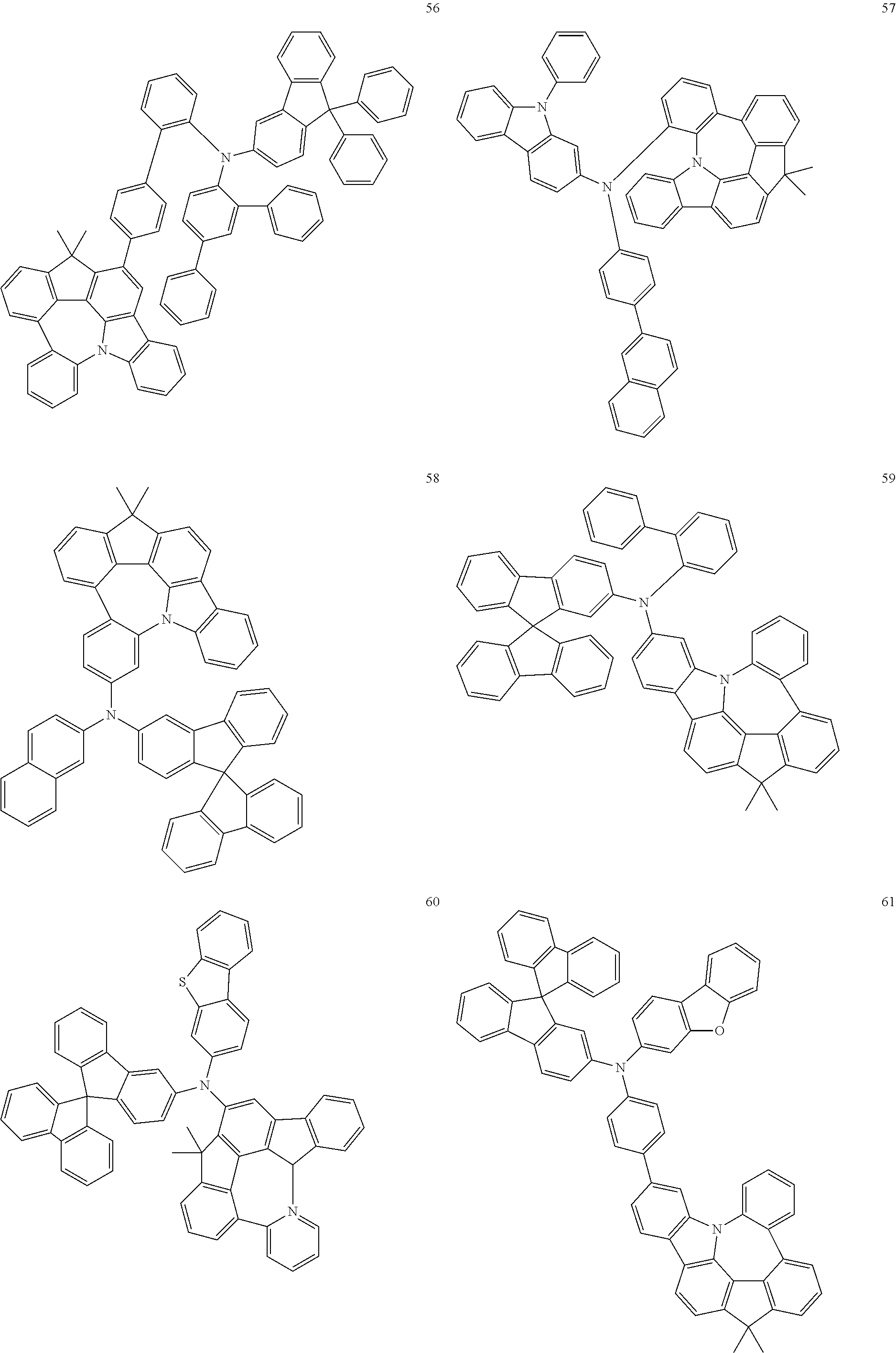

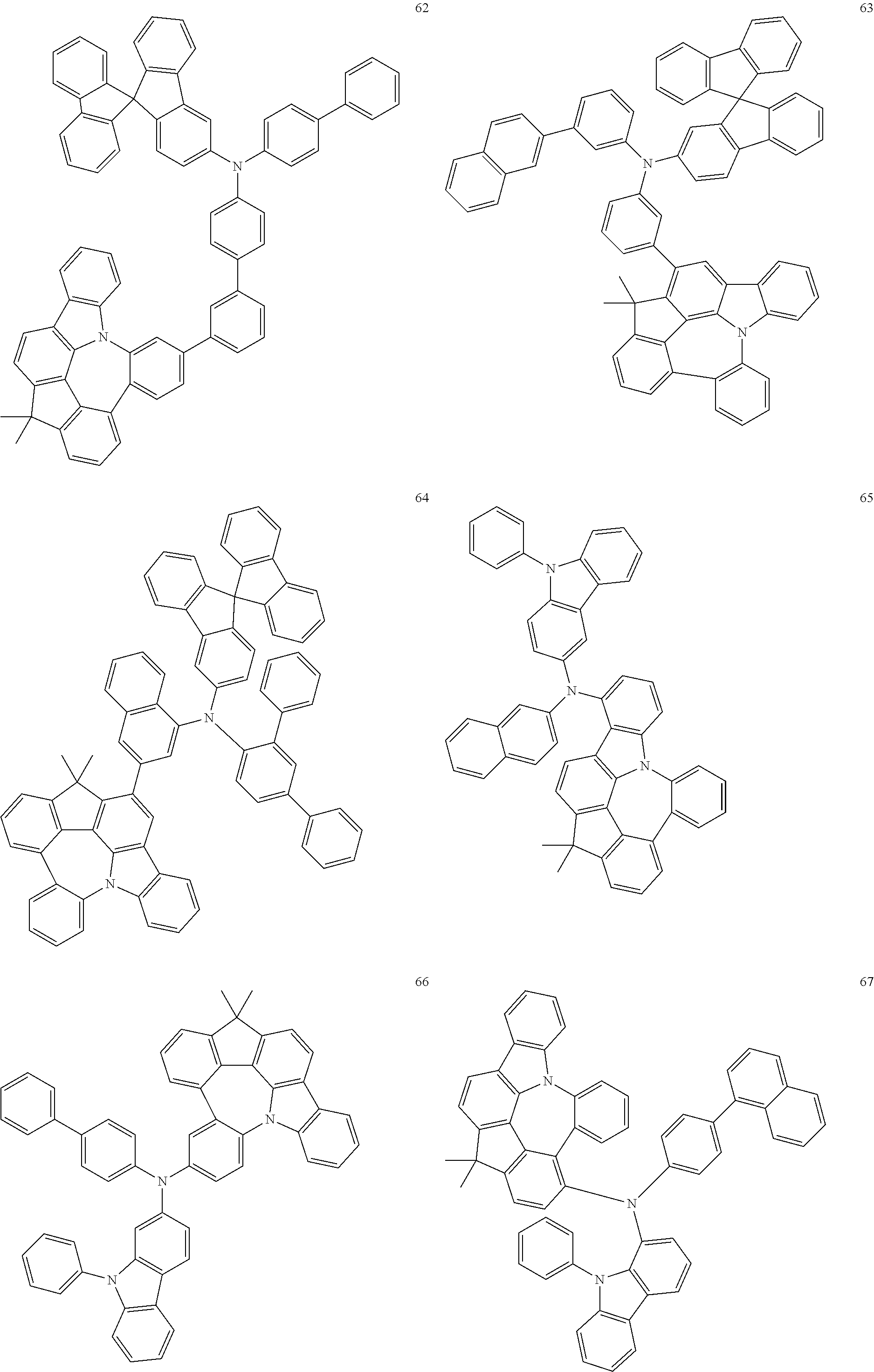

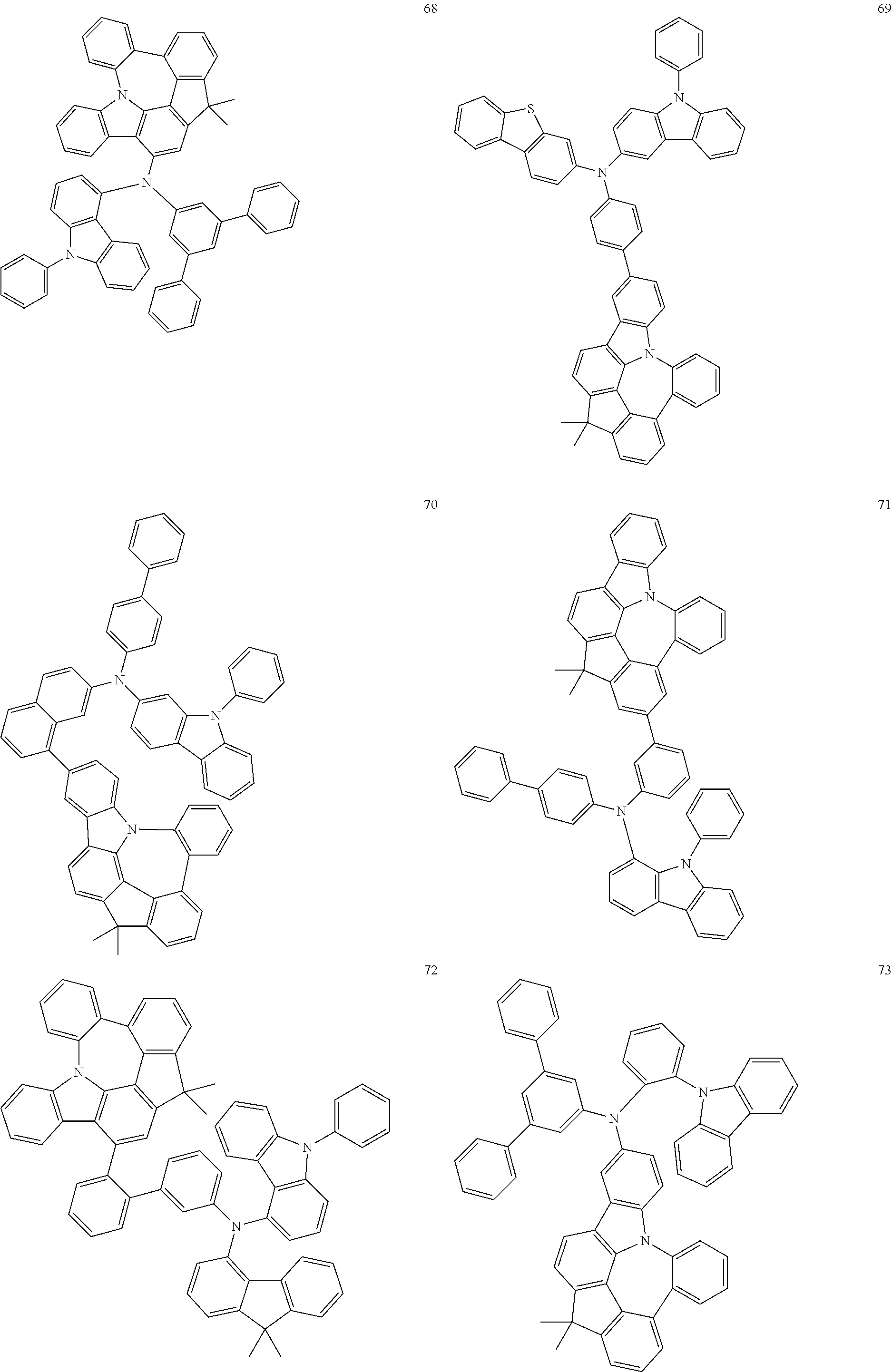

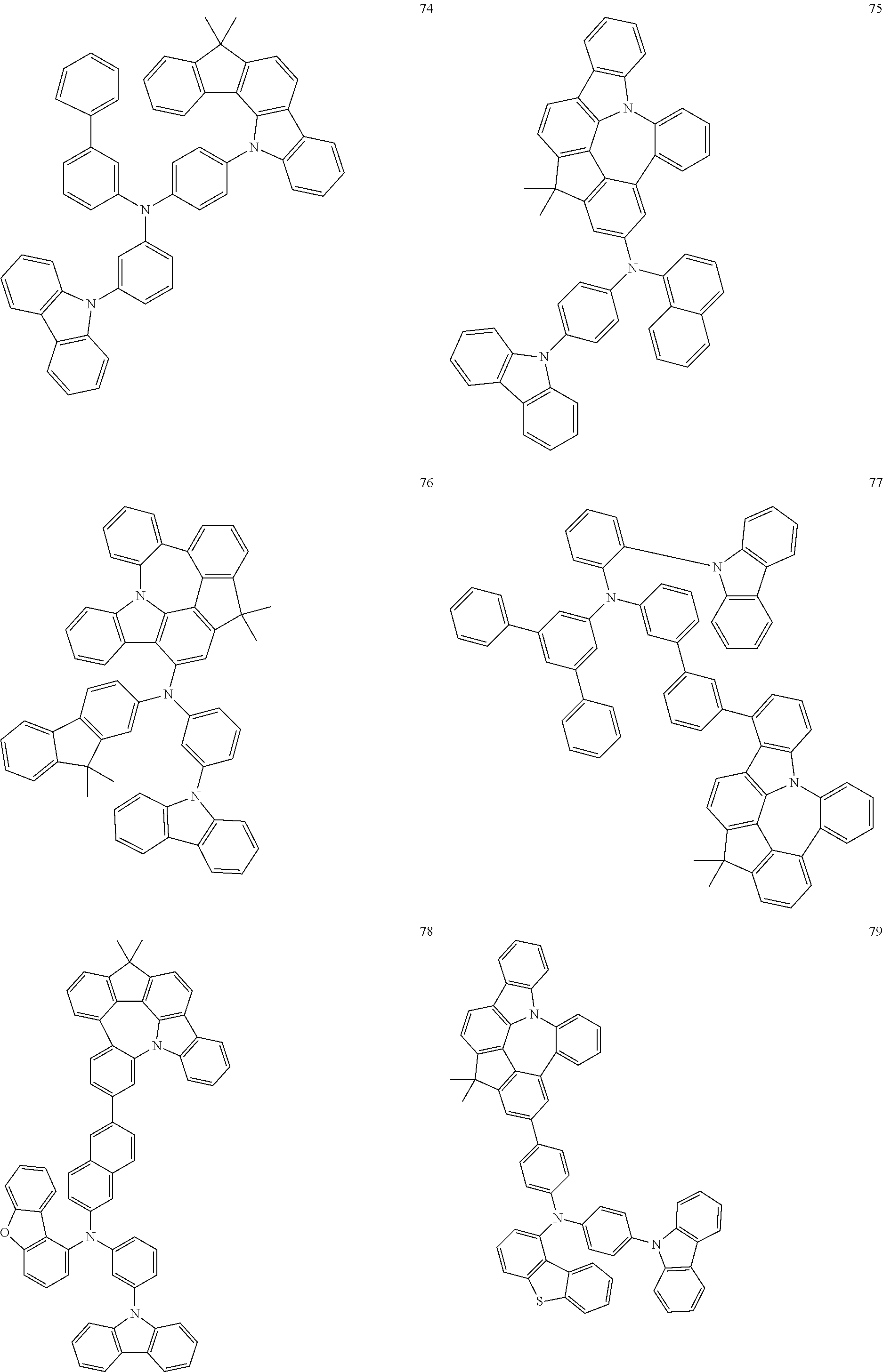

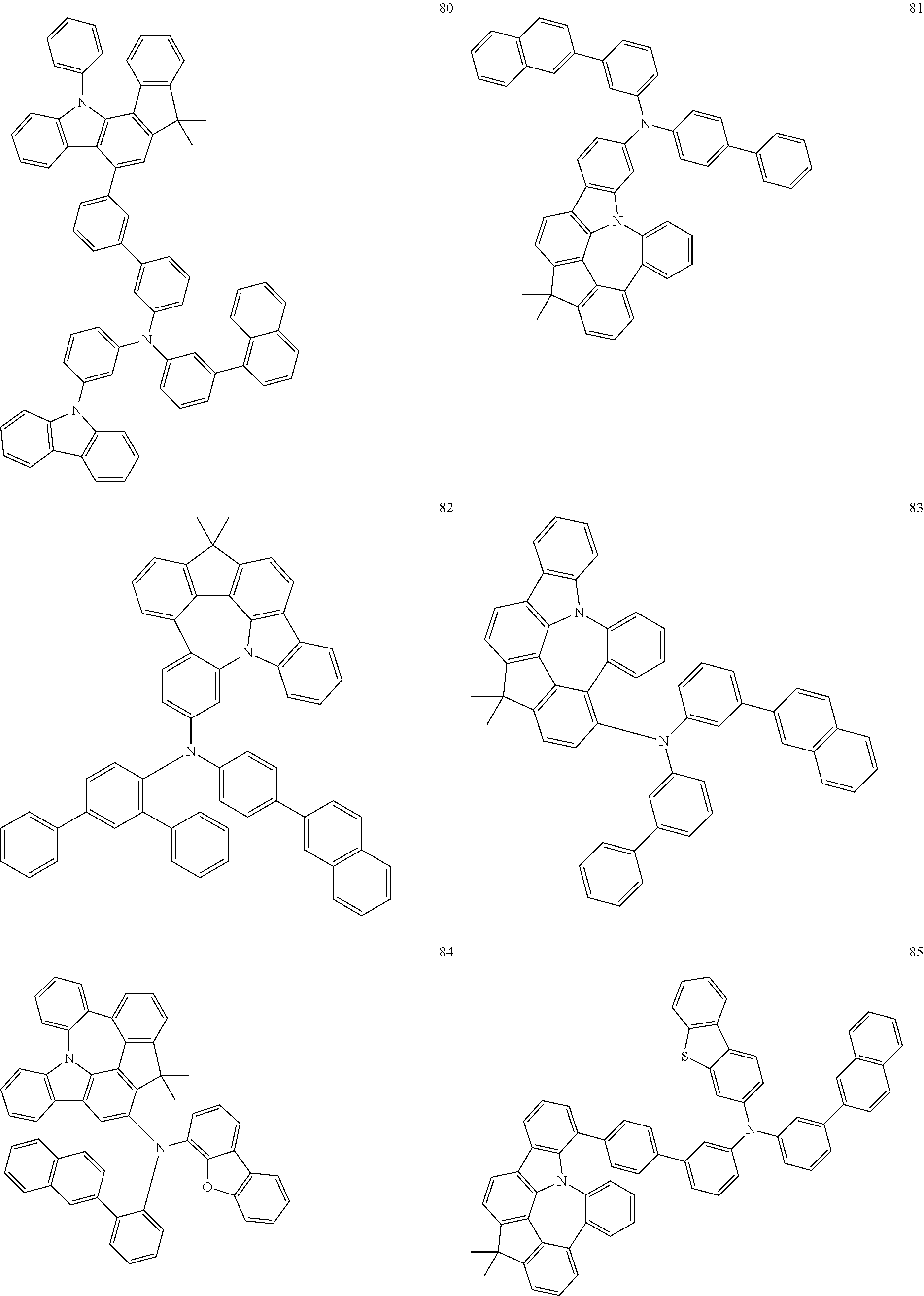

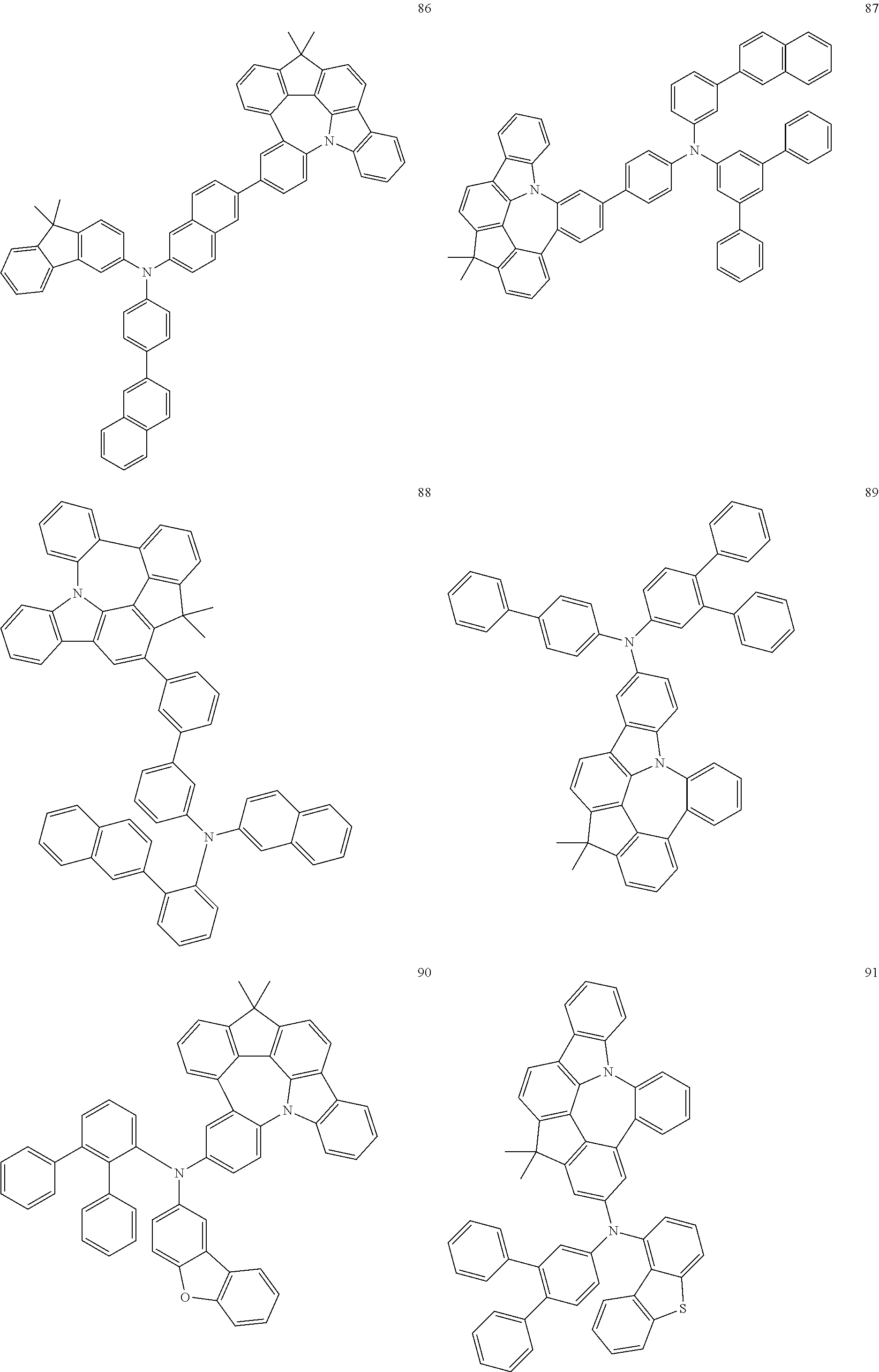

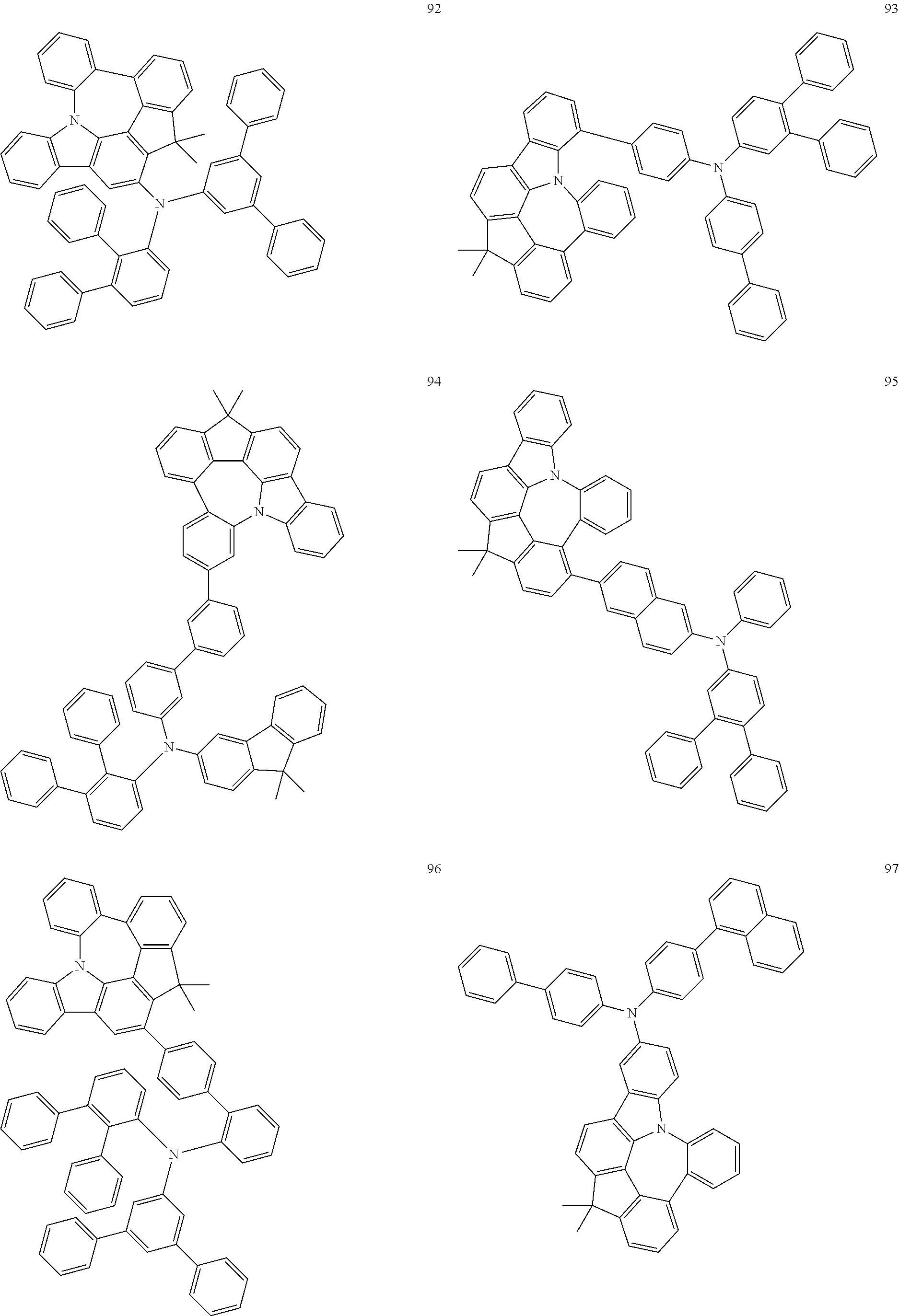

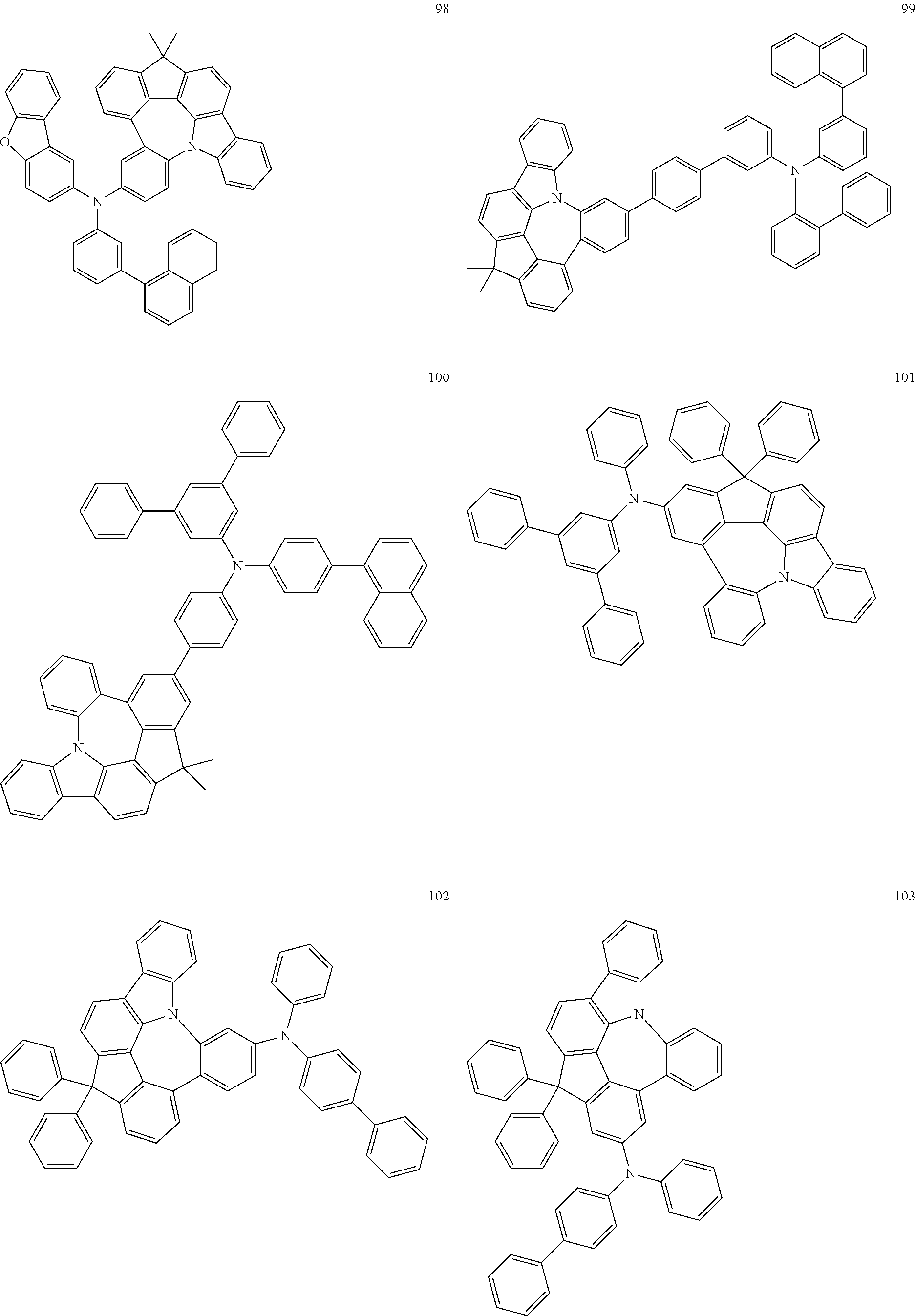

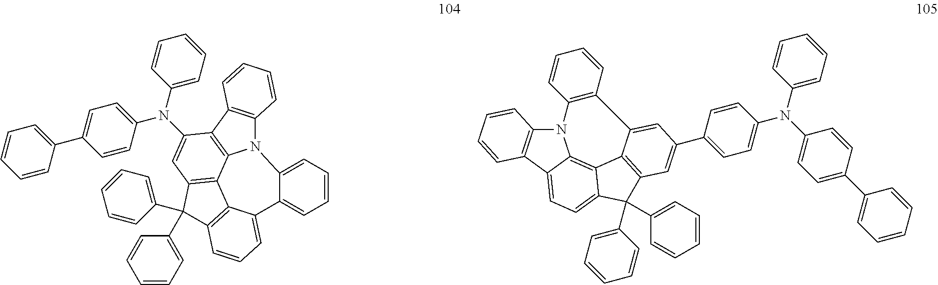 [SMILES: c1ccc(-c2ccc(N(c3ccccc3)c3cc4c5c6c3c3ccccc3n6-c3ccccc3-c3cccc(c3-5)C4(c3ccccc3)c3ccccc3)cc2)cc1 ; c1ccc(-c2ccc(N(c3ccccc3)c3ccc(-c4cc5c6c(c4)C(c4ccccc4)(c4ccccc4)c4ccc7c8ccccc8n(c7c4-6)-c4ccccc4-5)cc3)cc2)cc1]

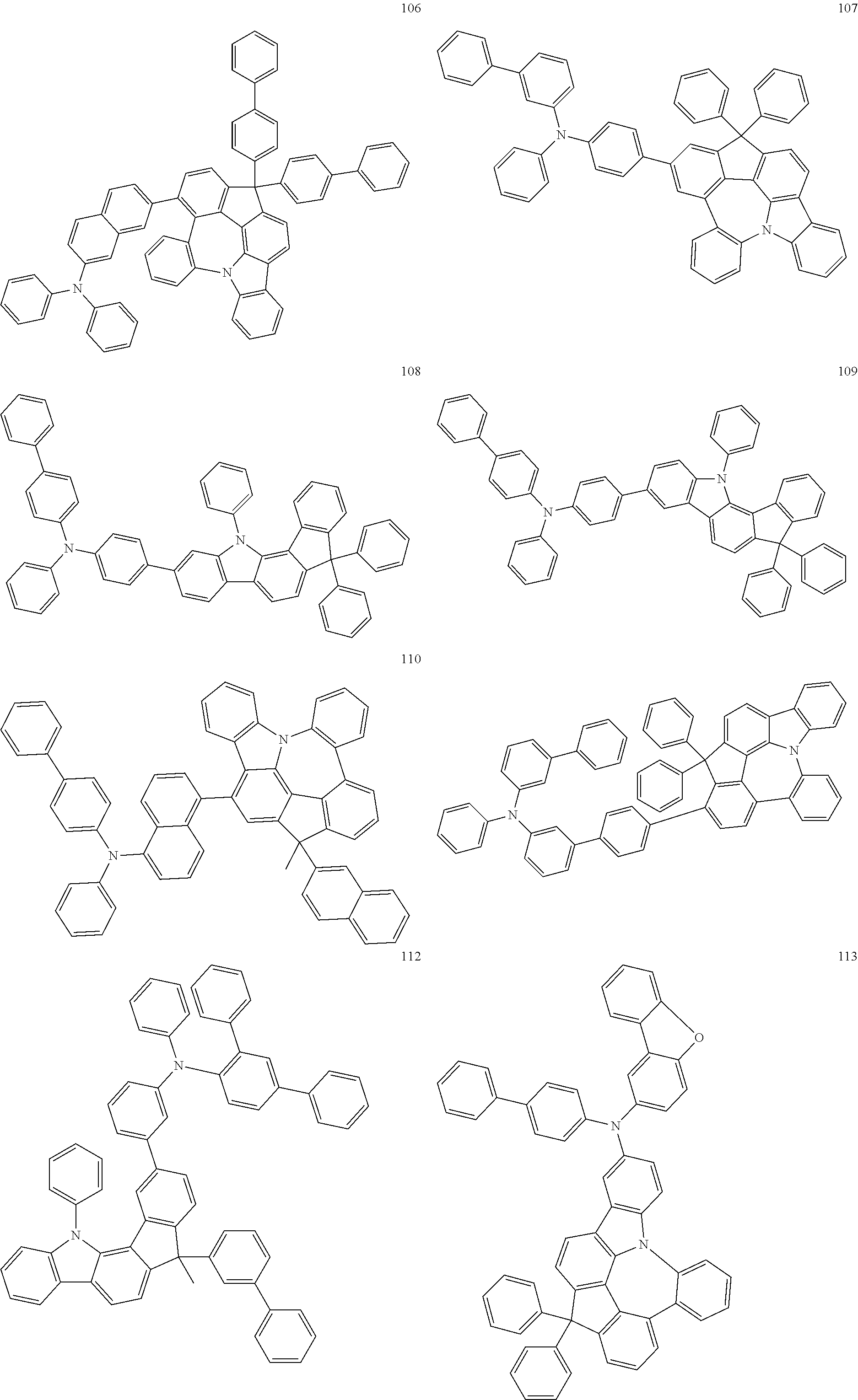

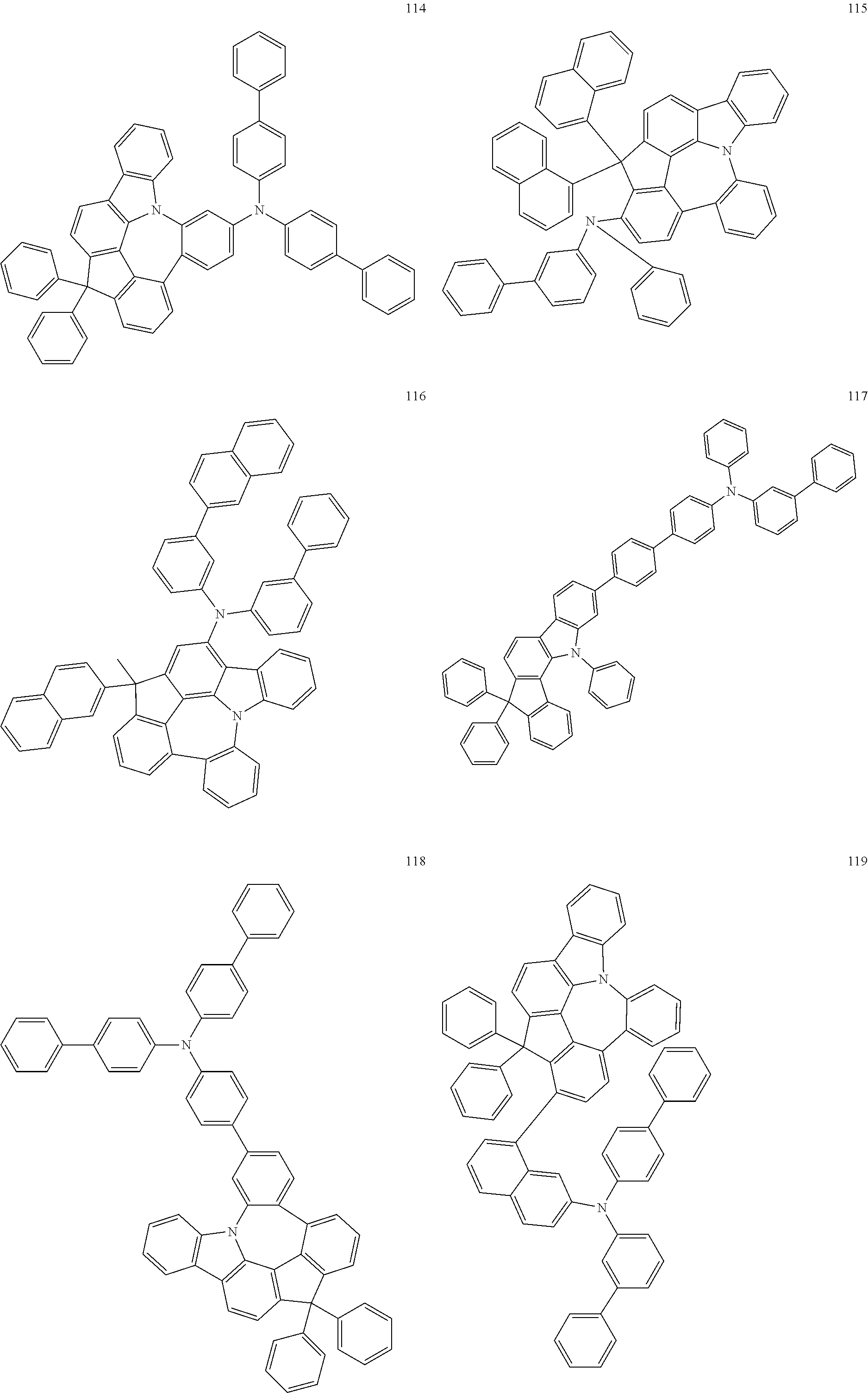

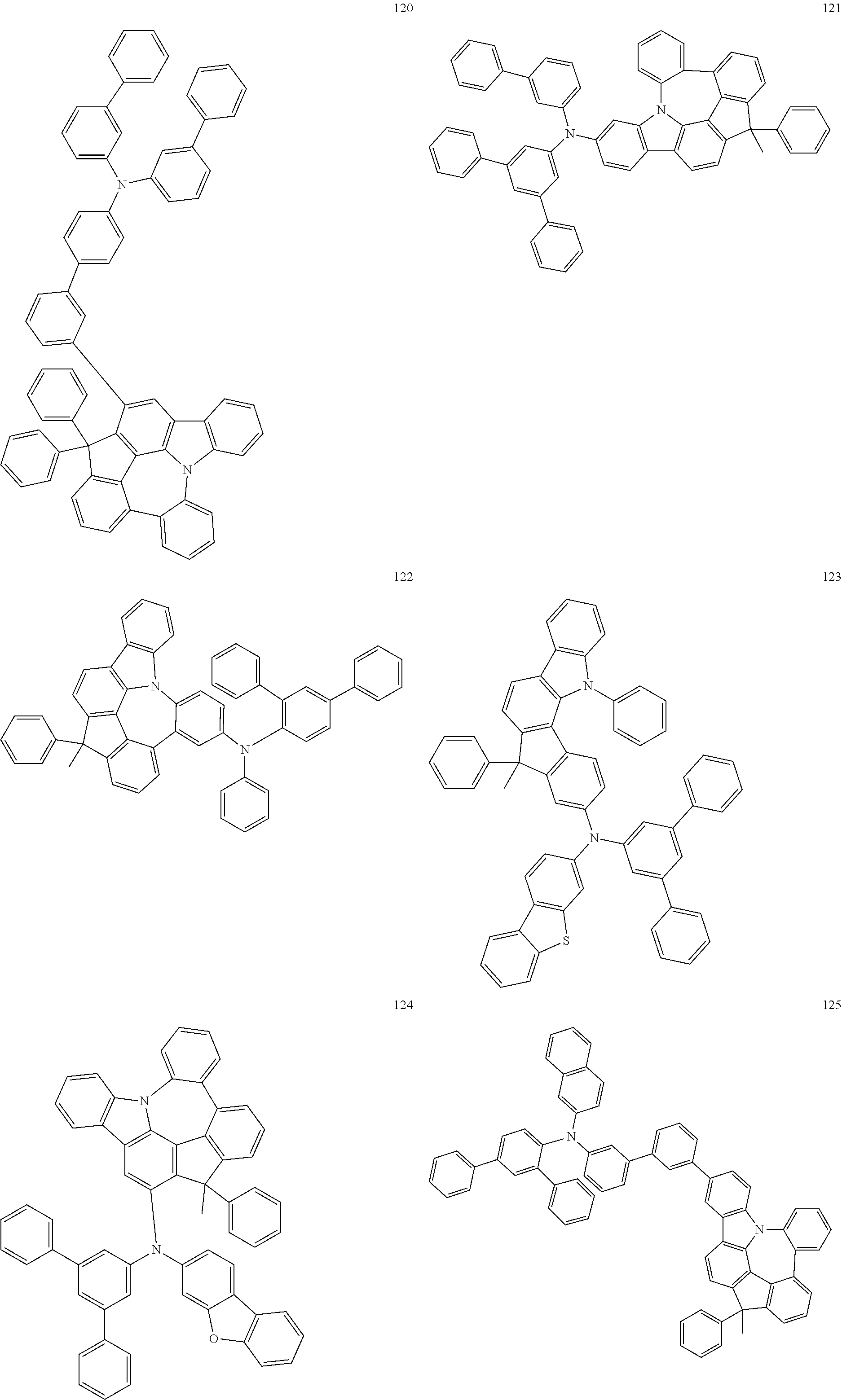

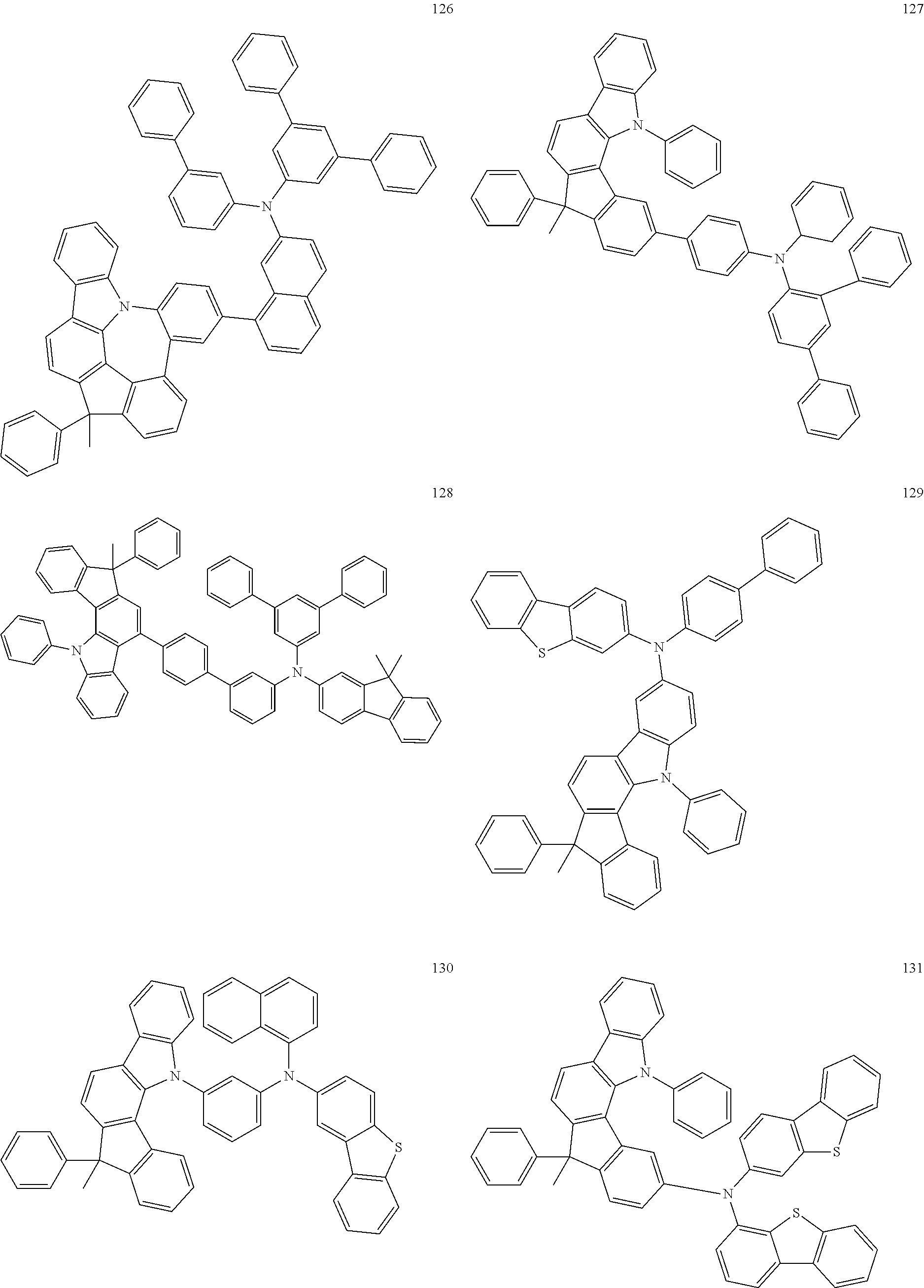

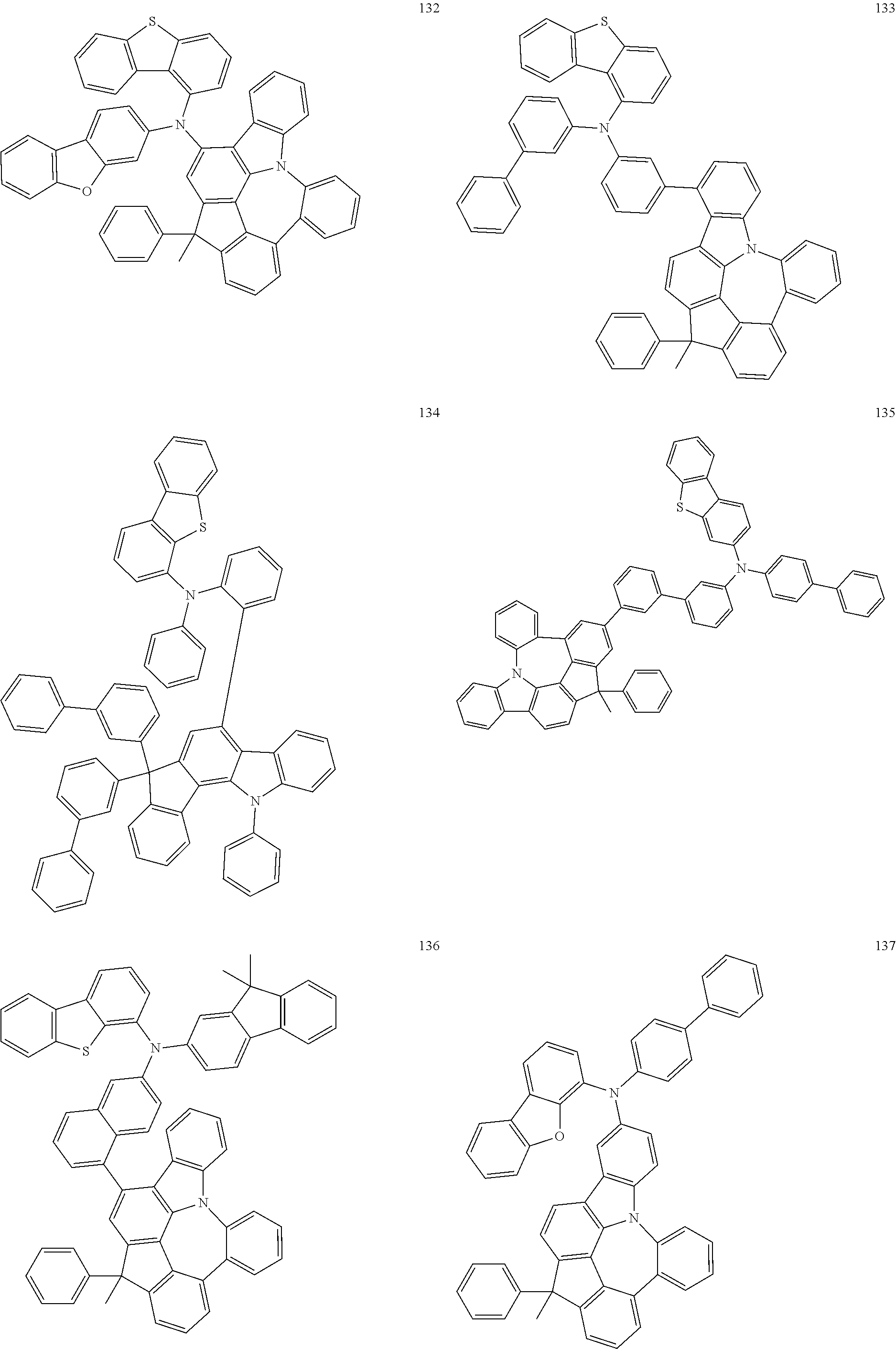

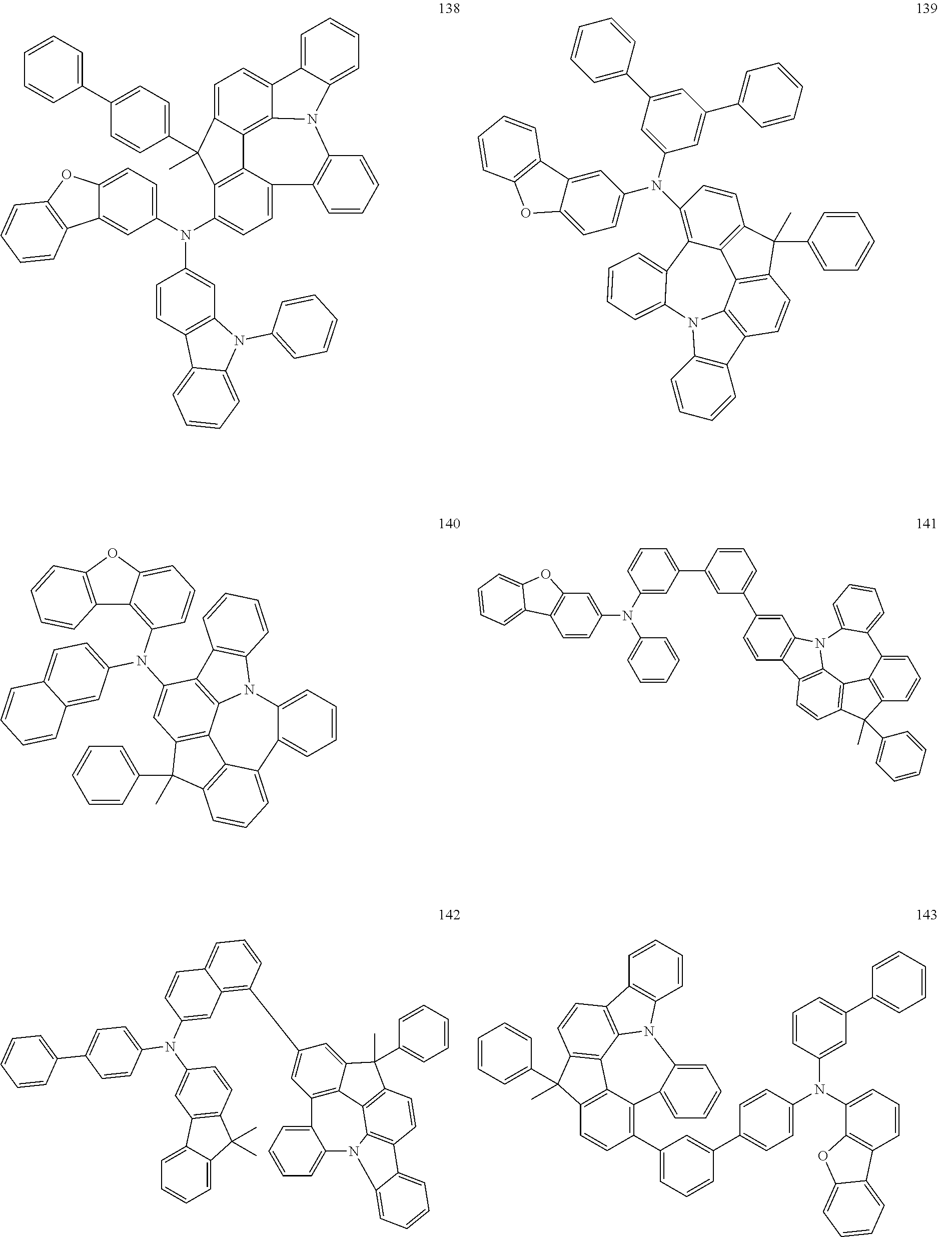

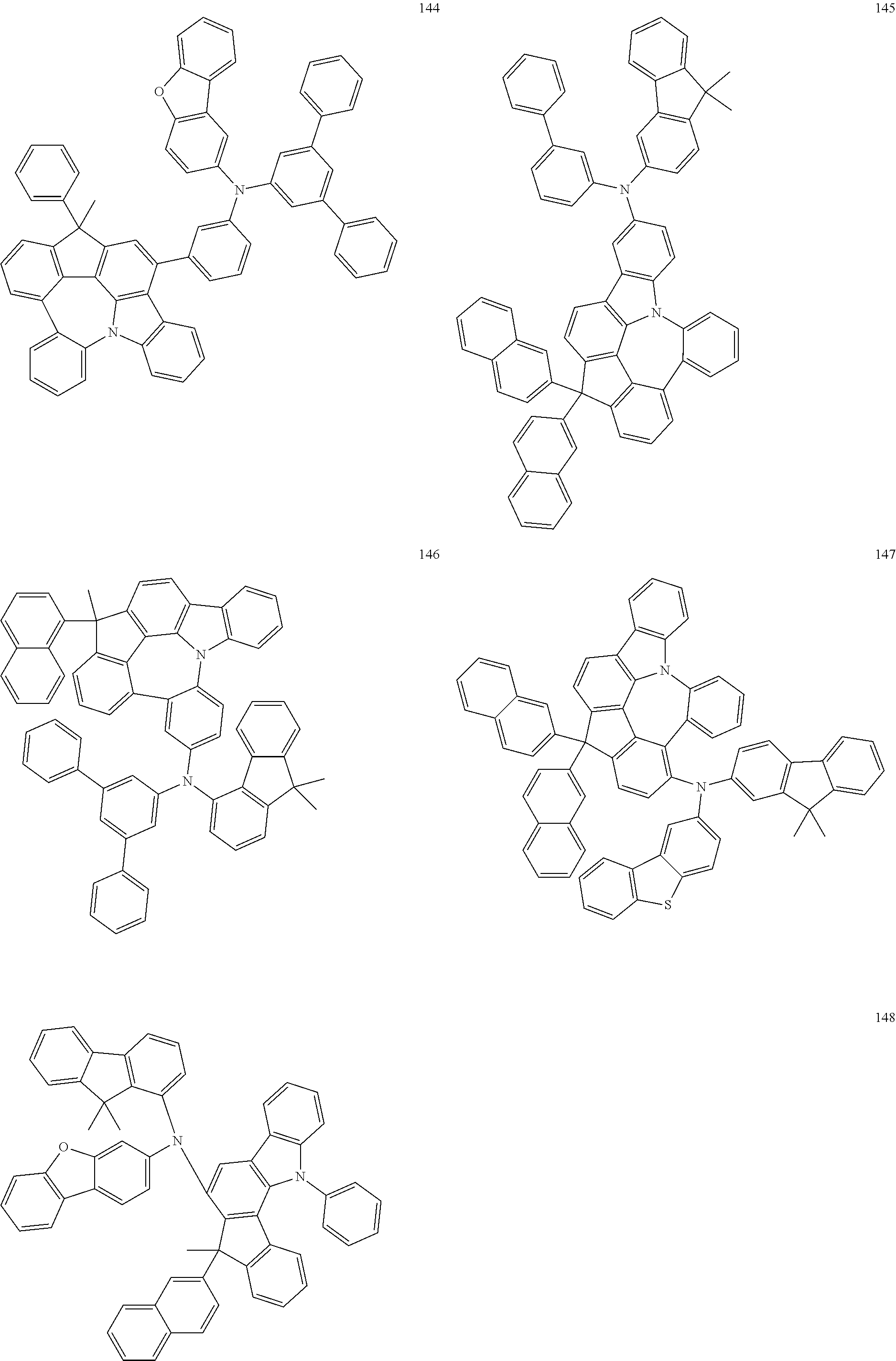

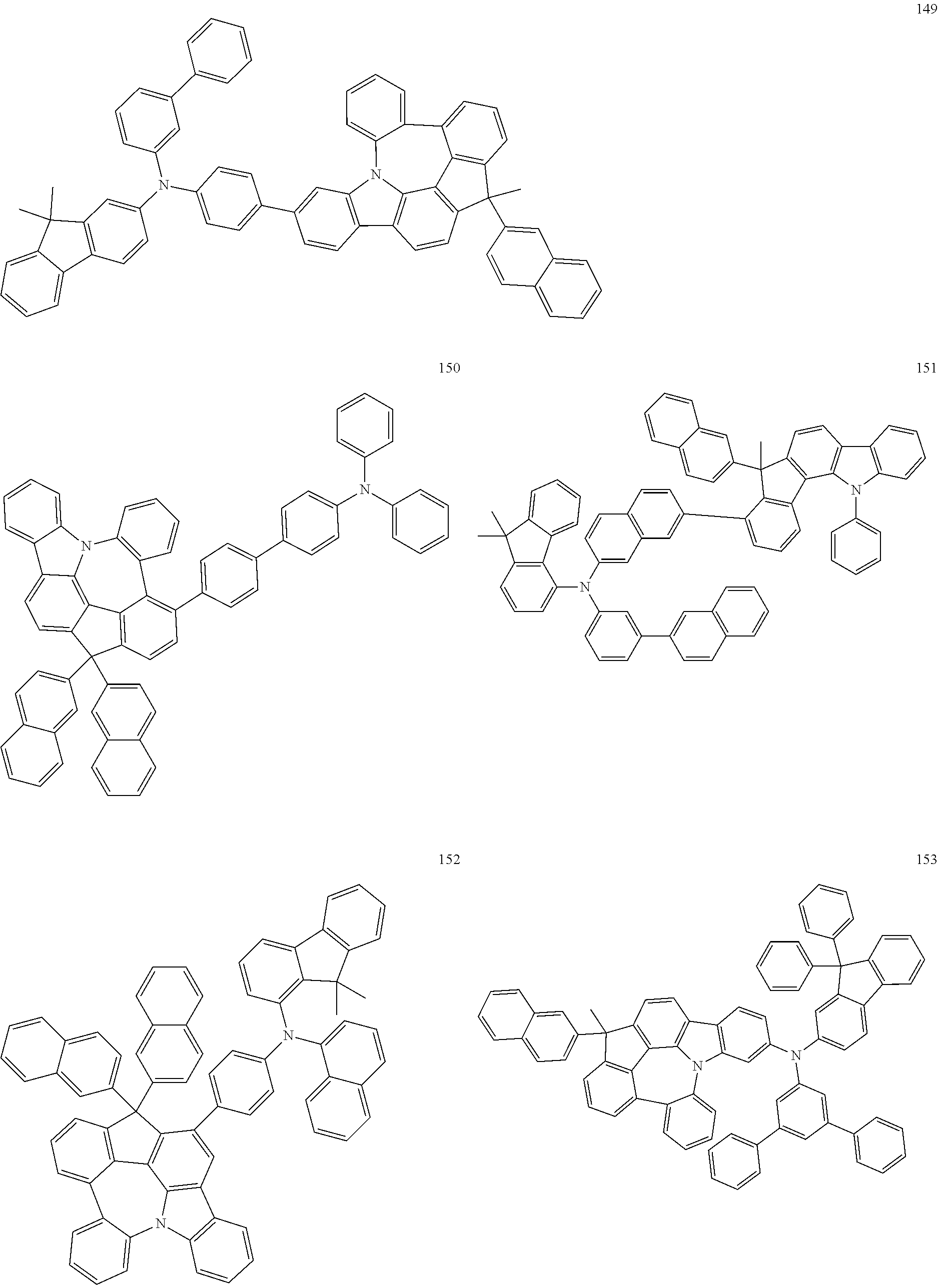

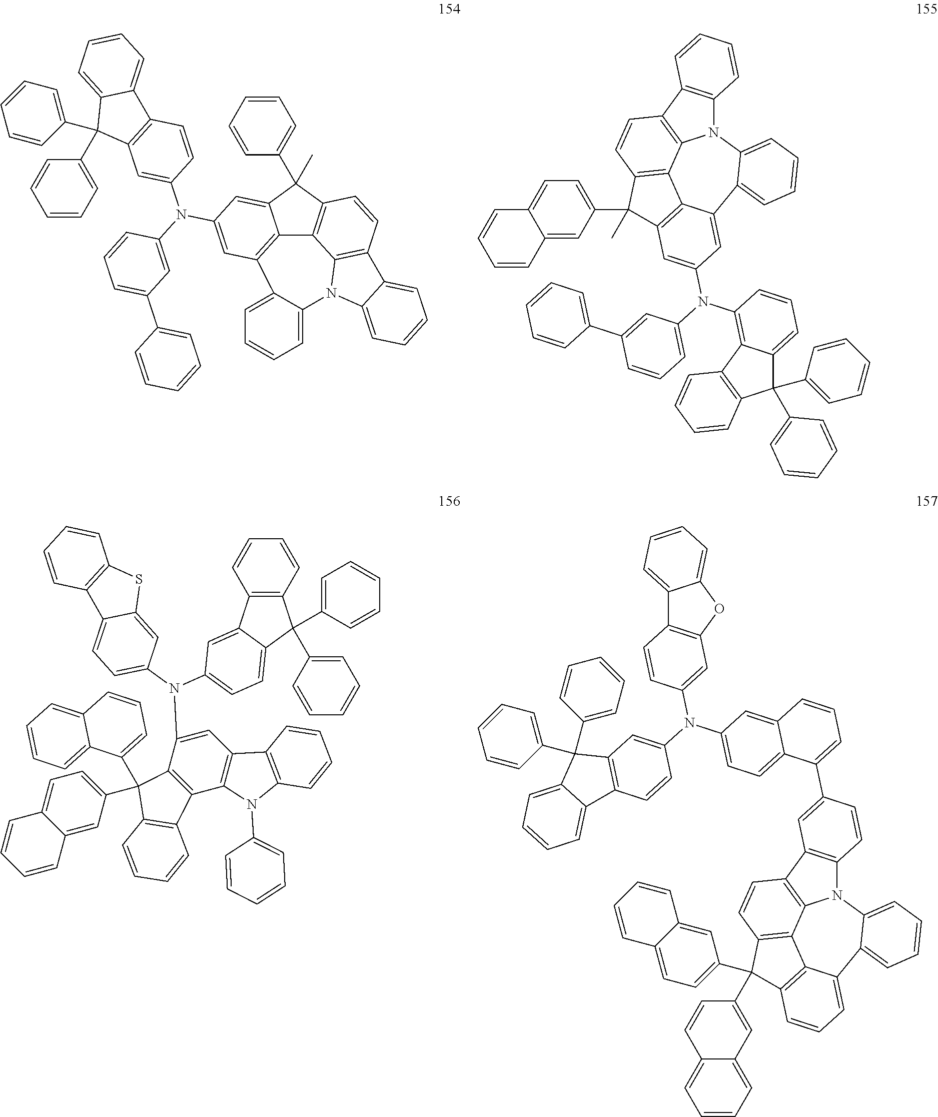

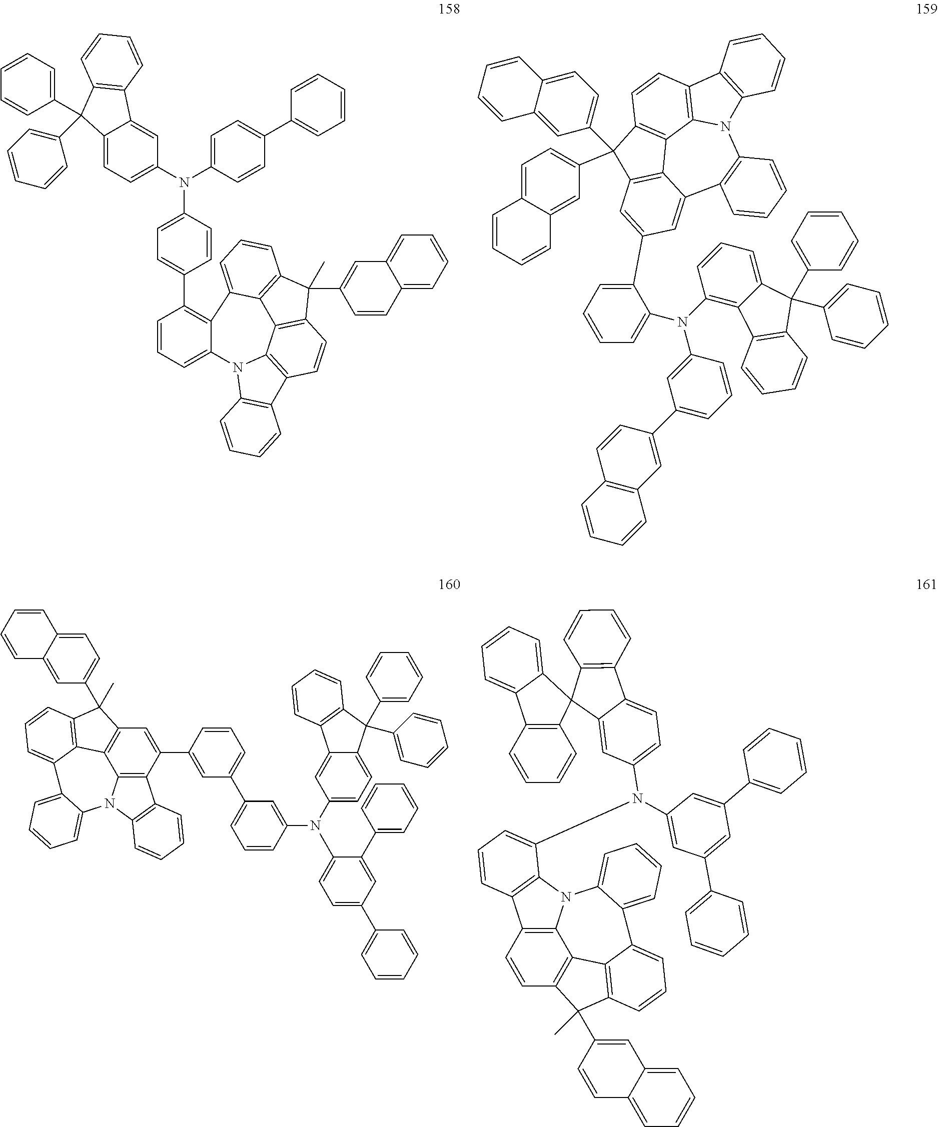

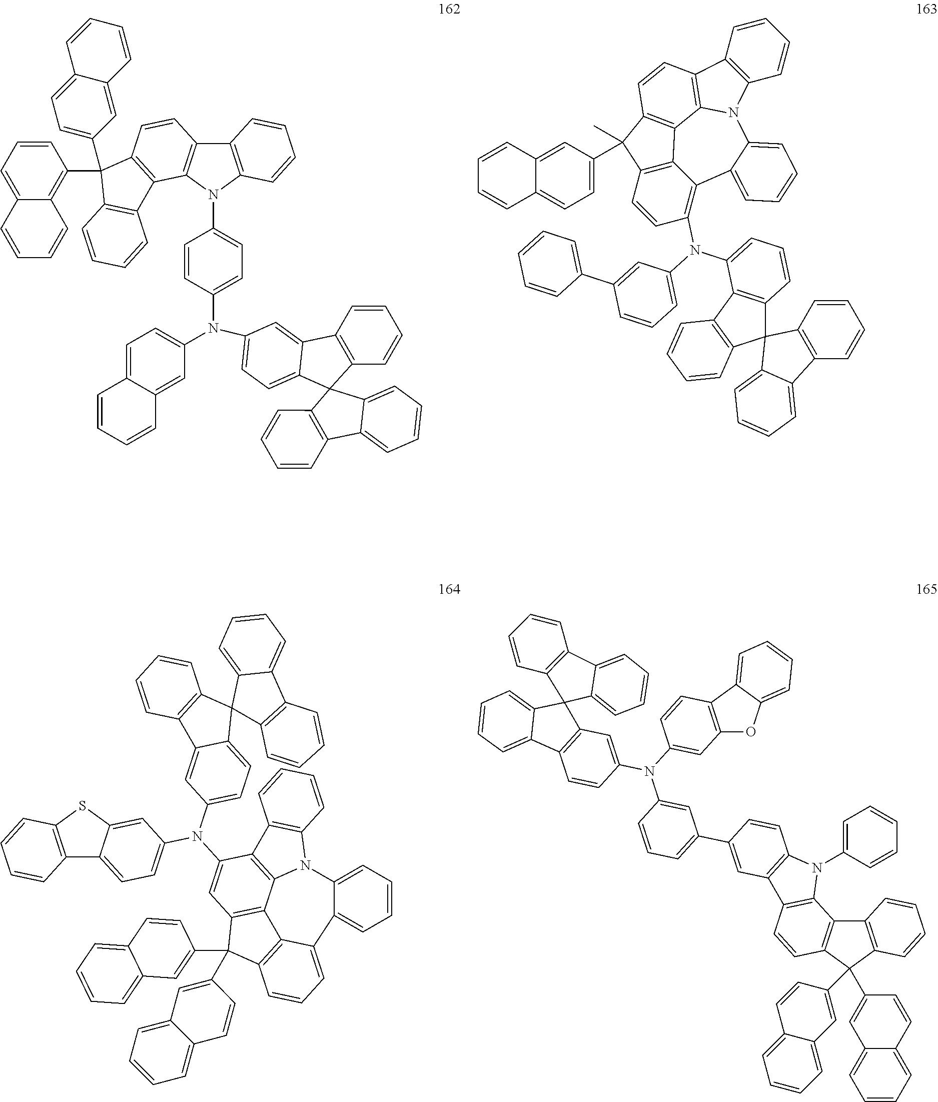

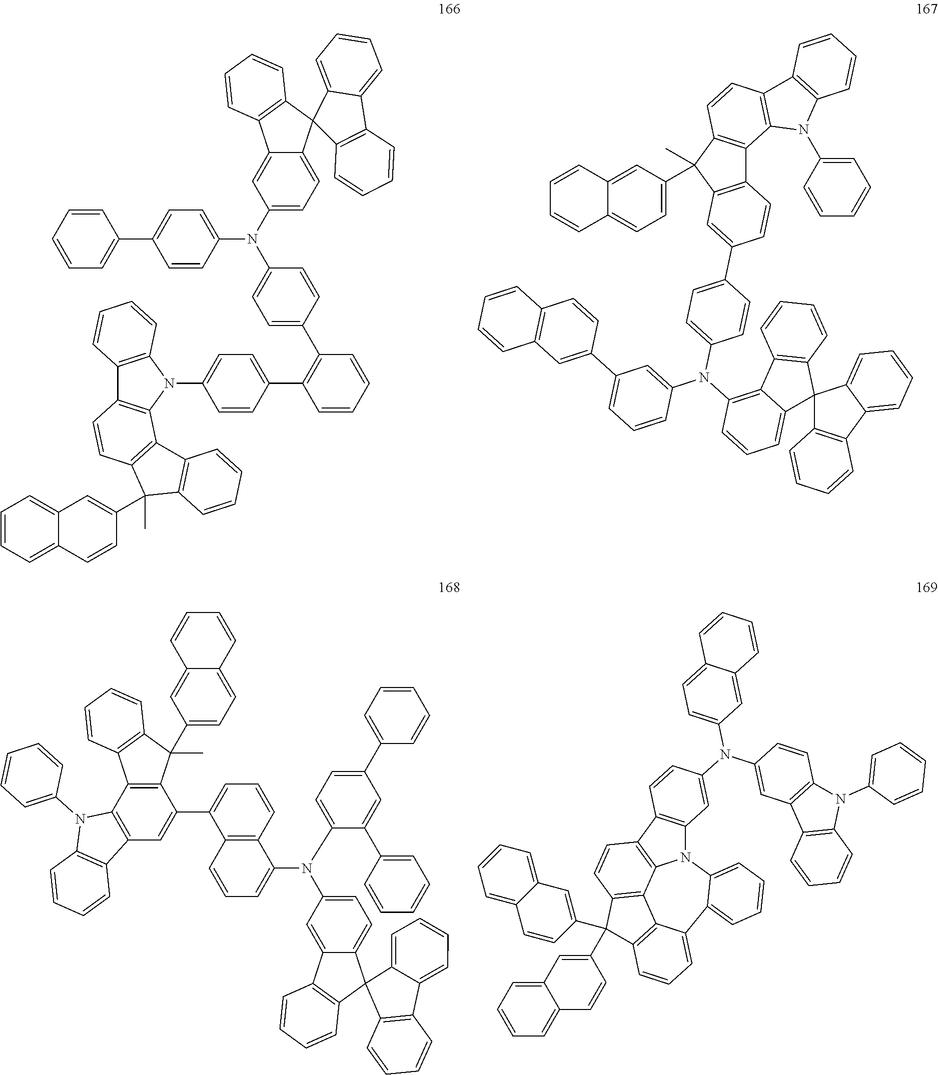

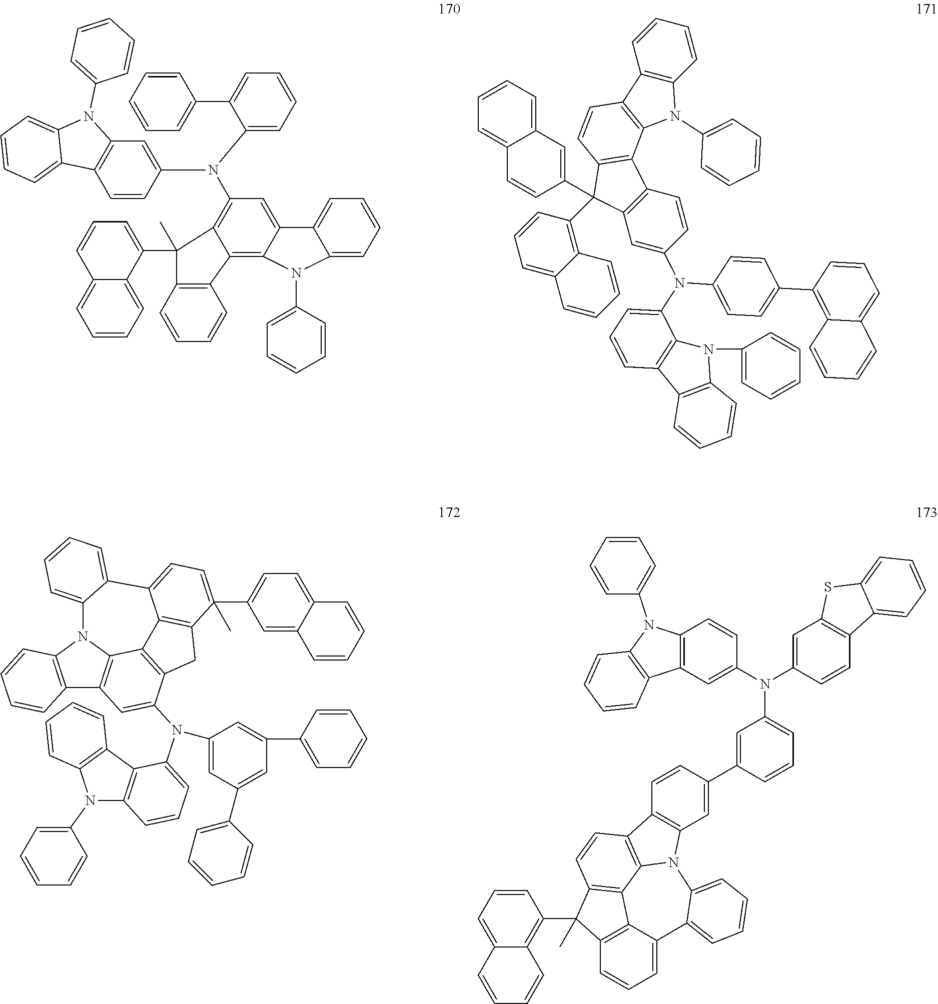

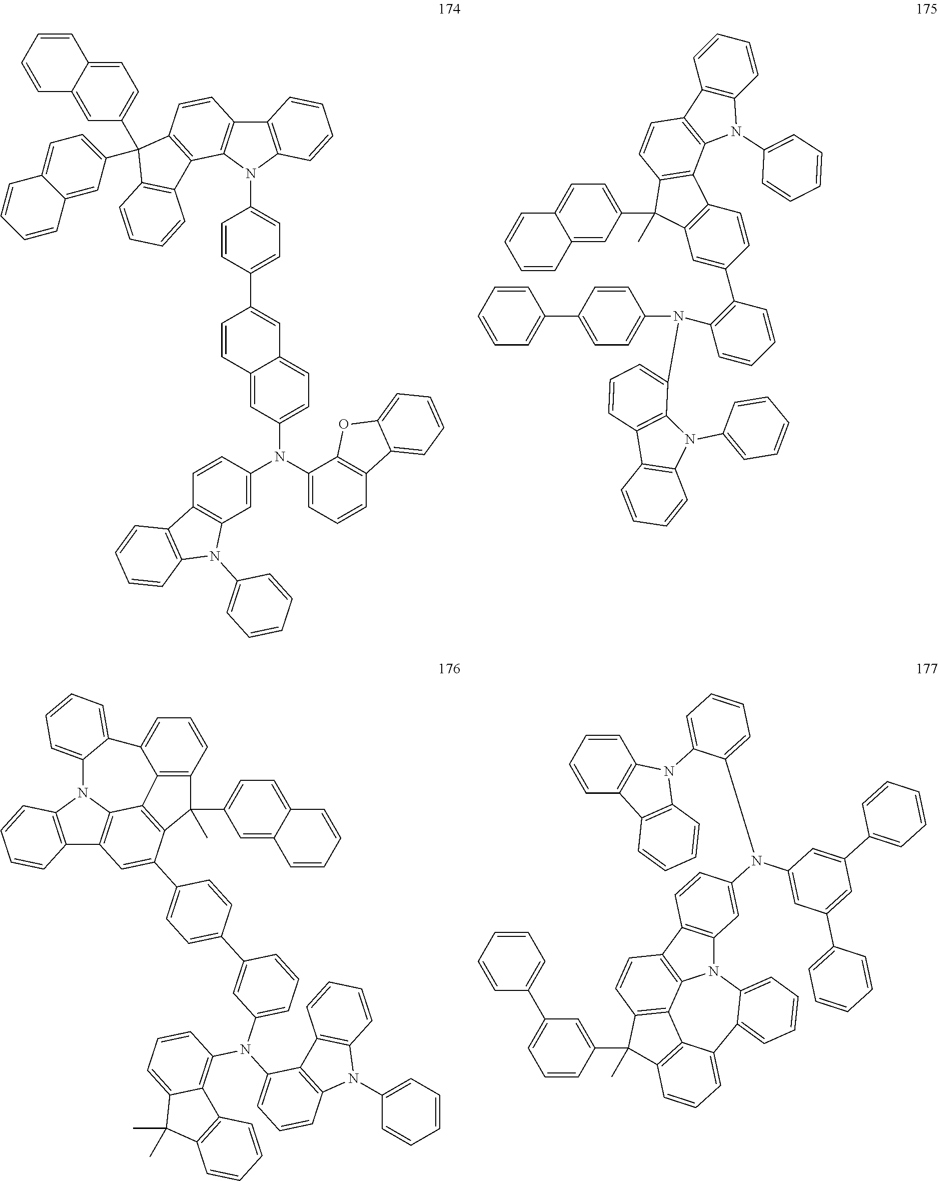

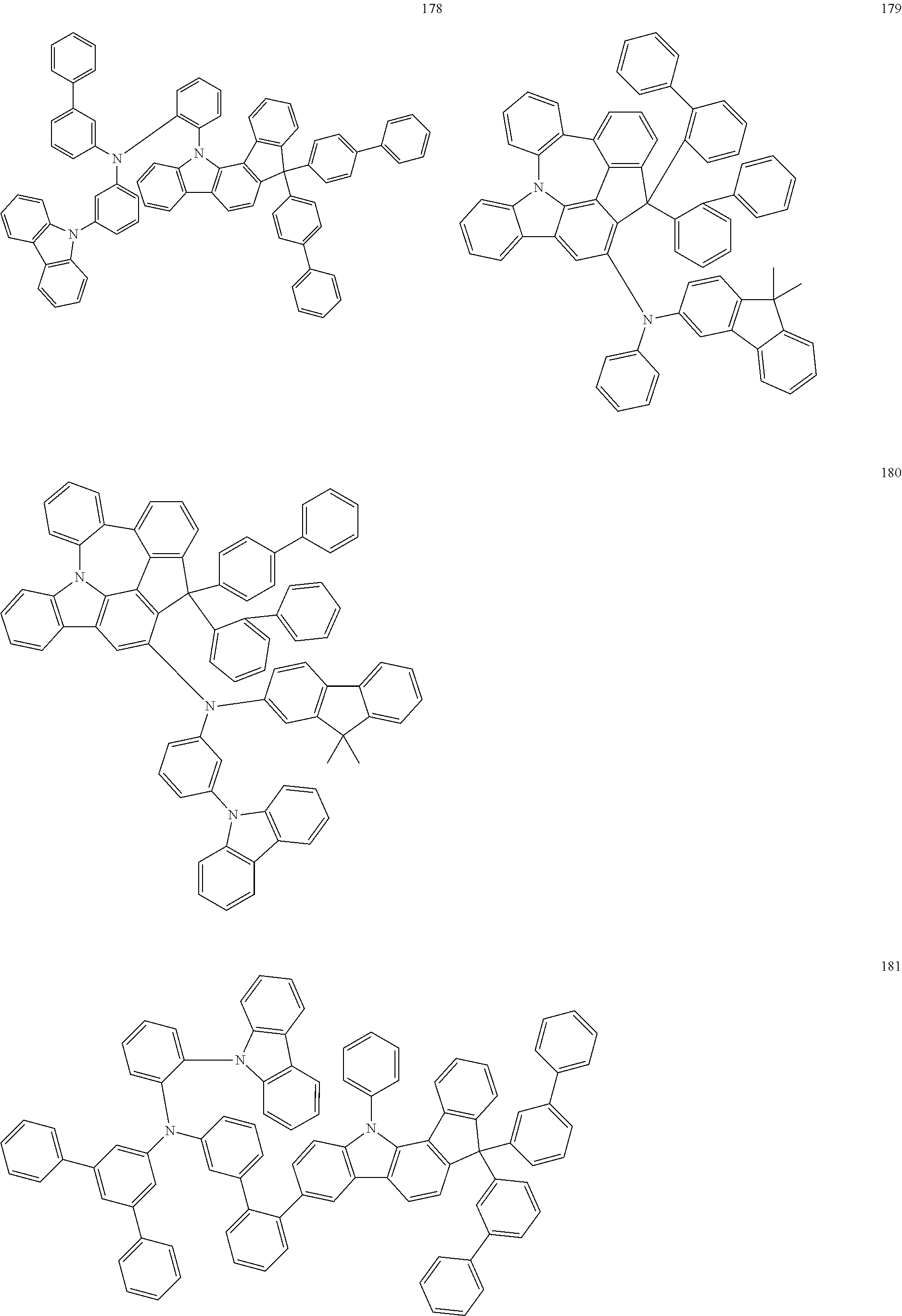

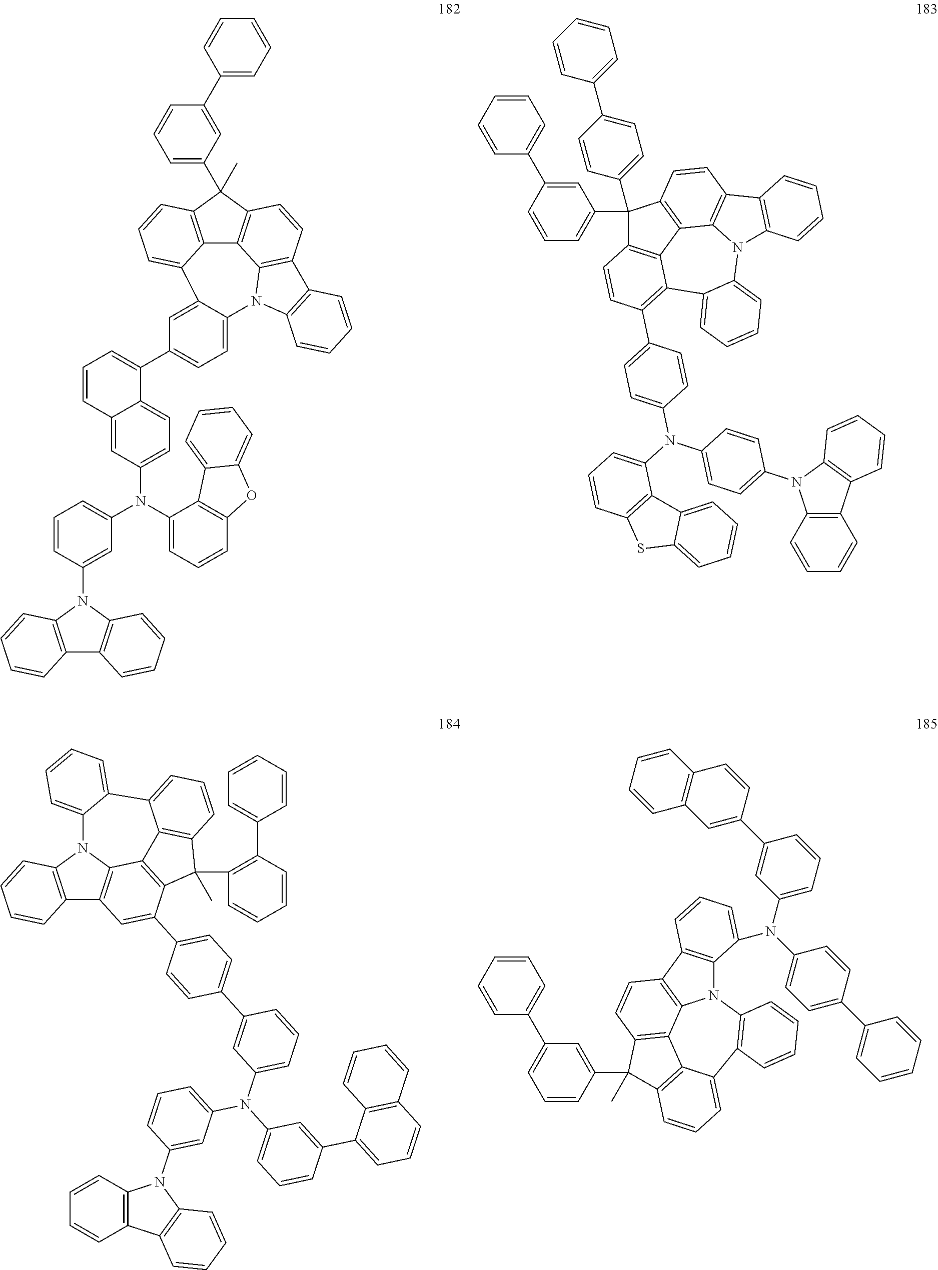

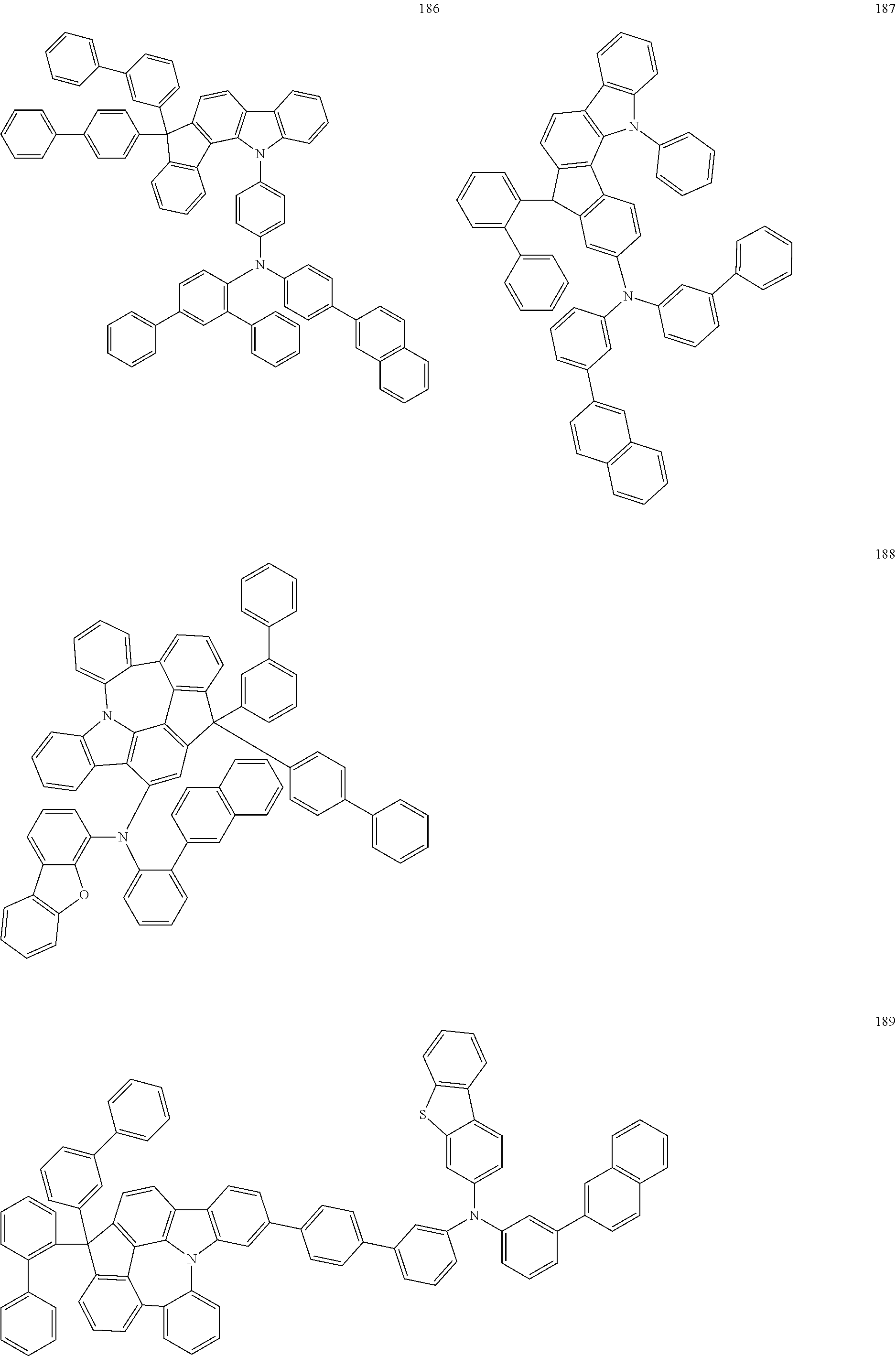

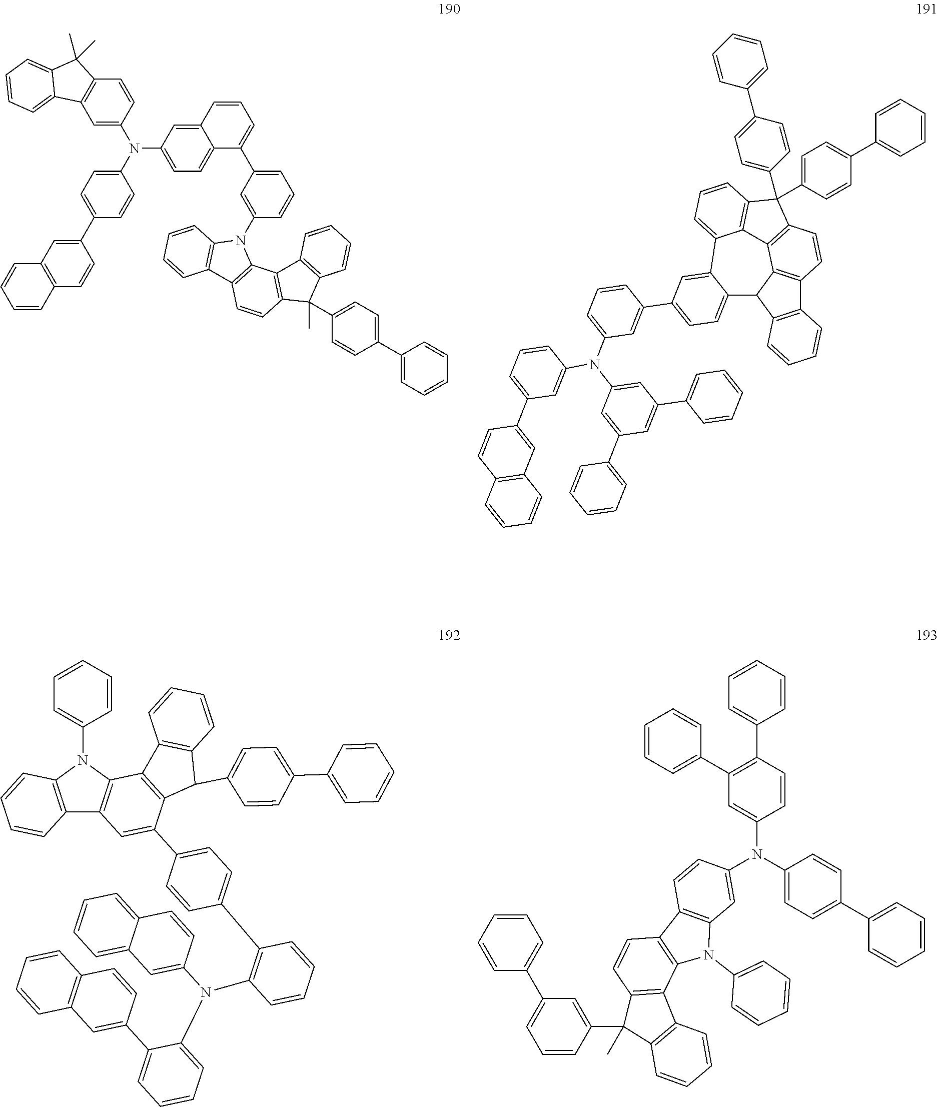

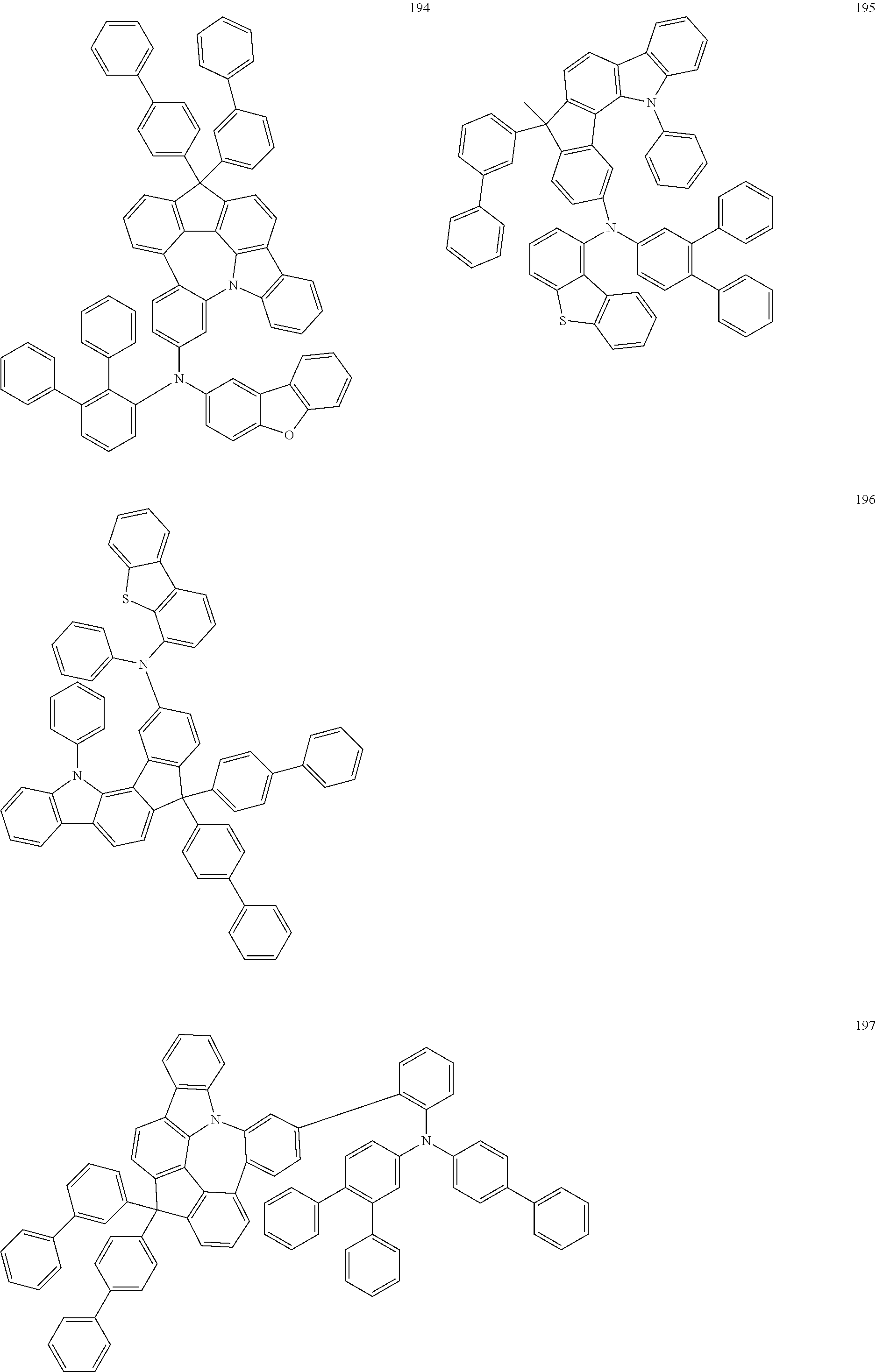

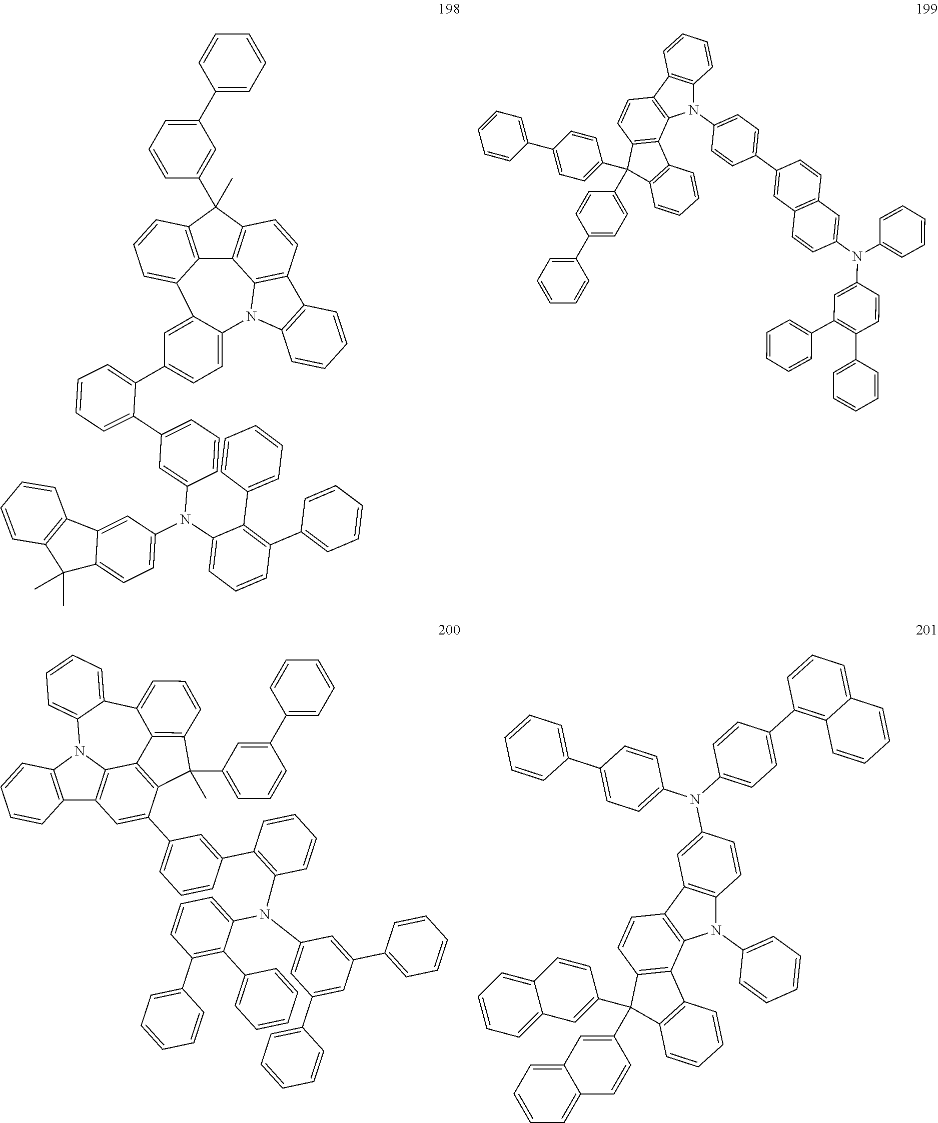

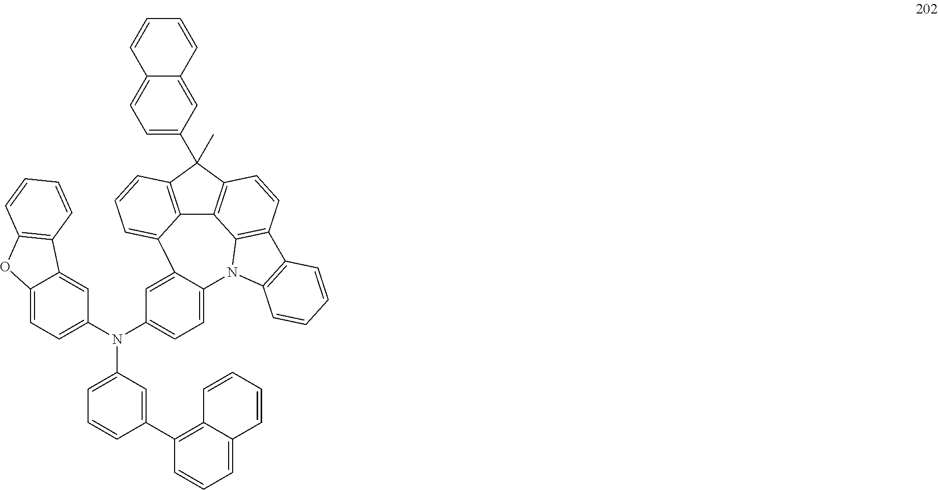 [SMILES: CC1(c2ccc3ccccc3c2)c2cccc3c2-c2c1ccc1c4ccccc4n(c21)-c1ccc(N(c2cccc(-c4cccc5ccccc45)c2)c2ccc4oc5ccccc5c4c2)cc1-3]

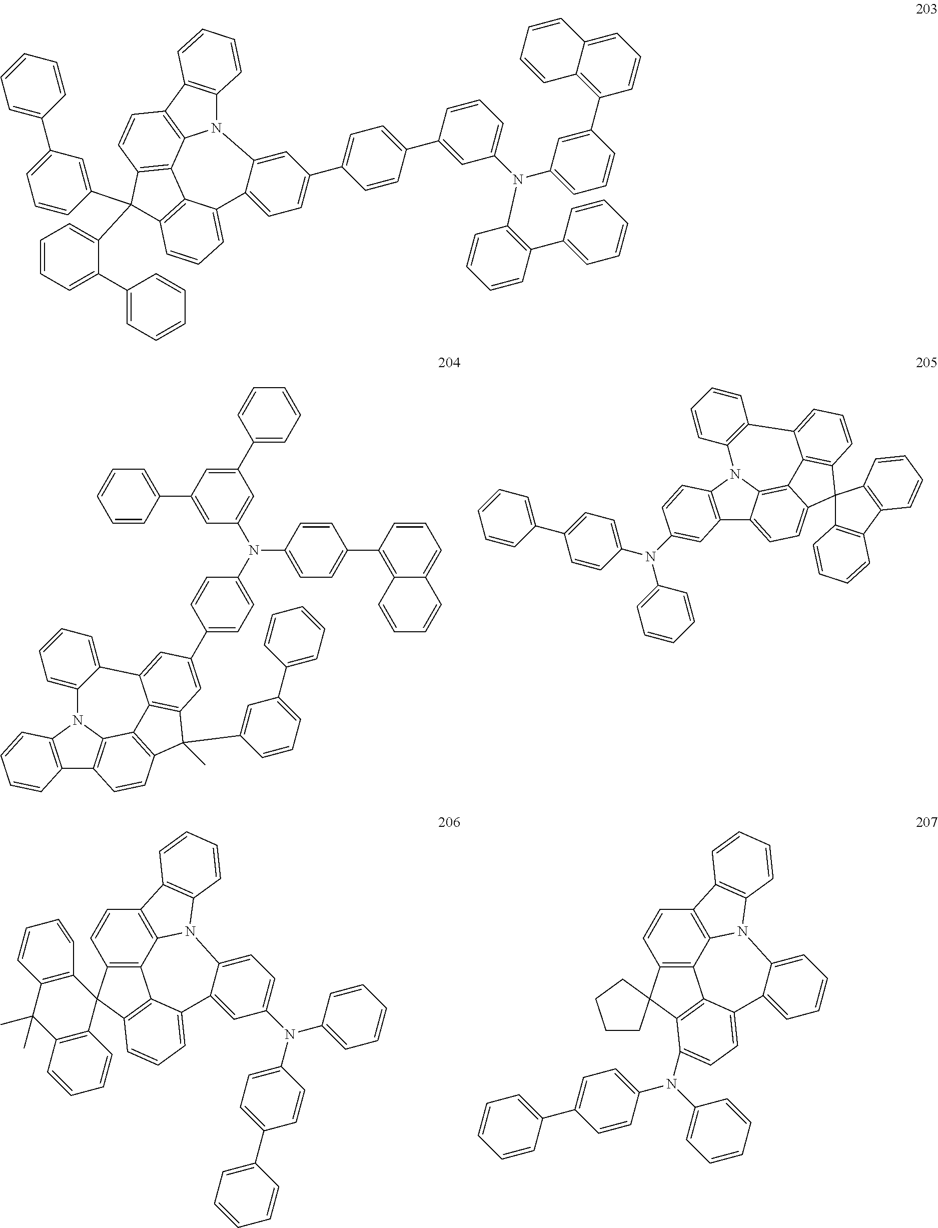

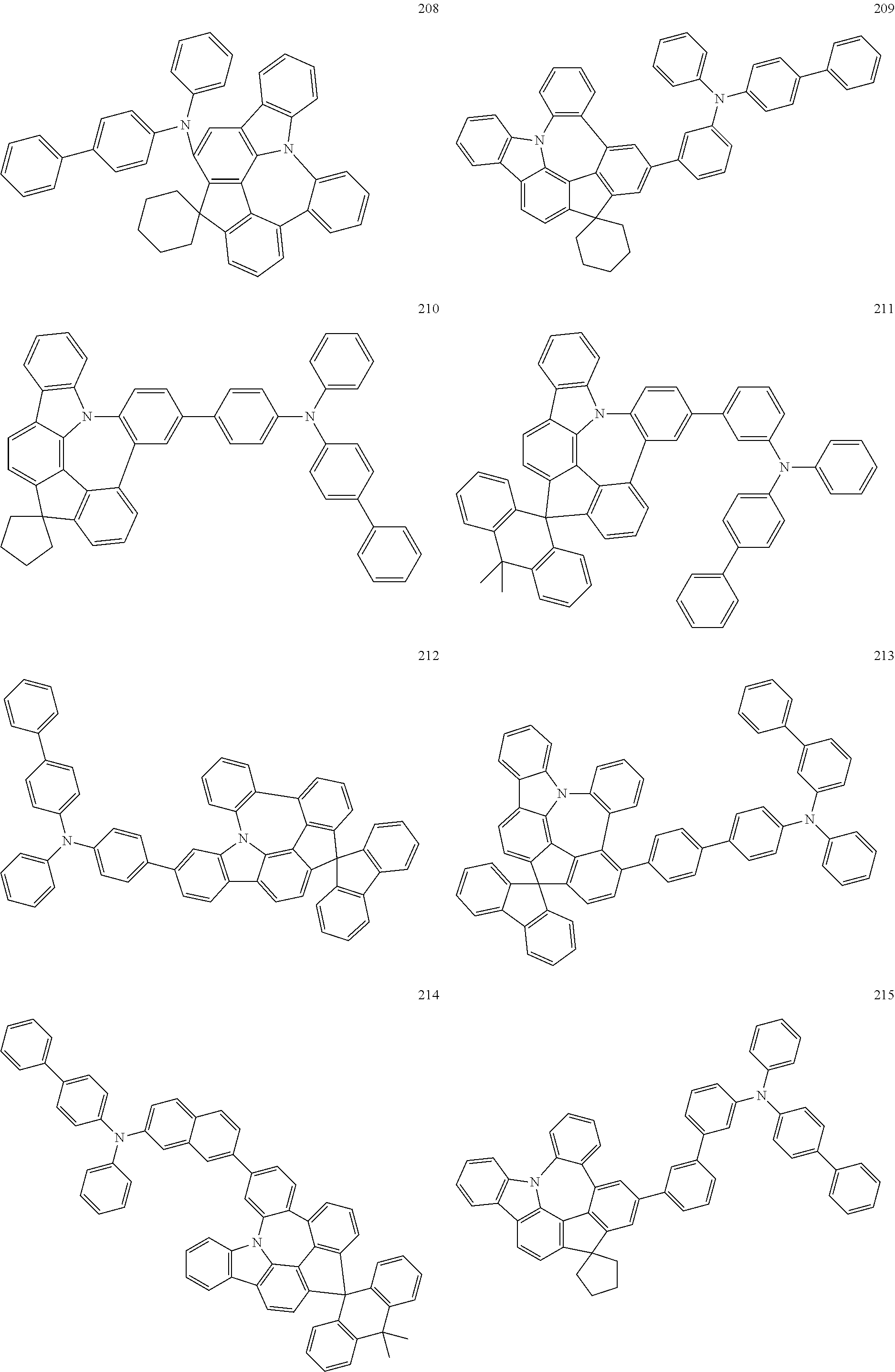

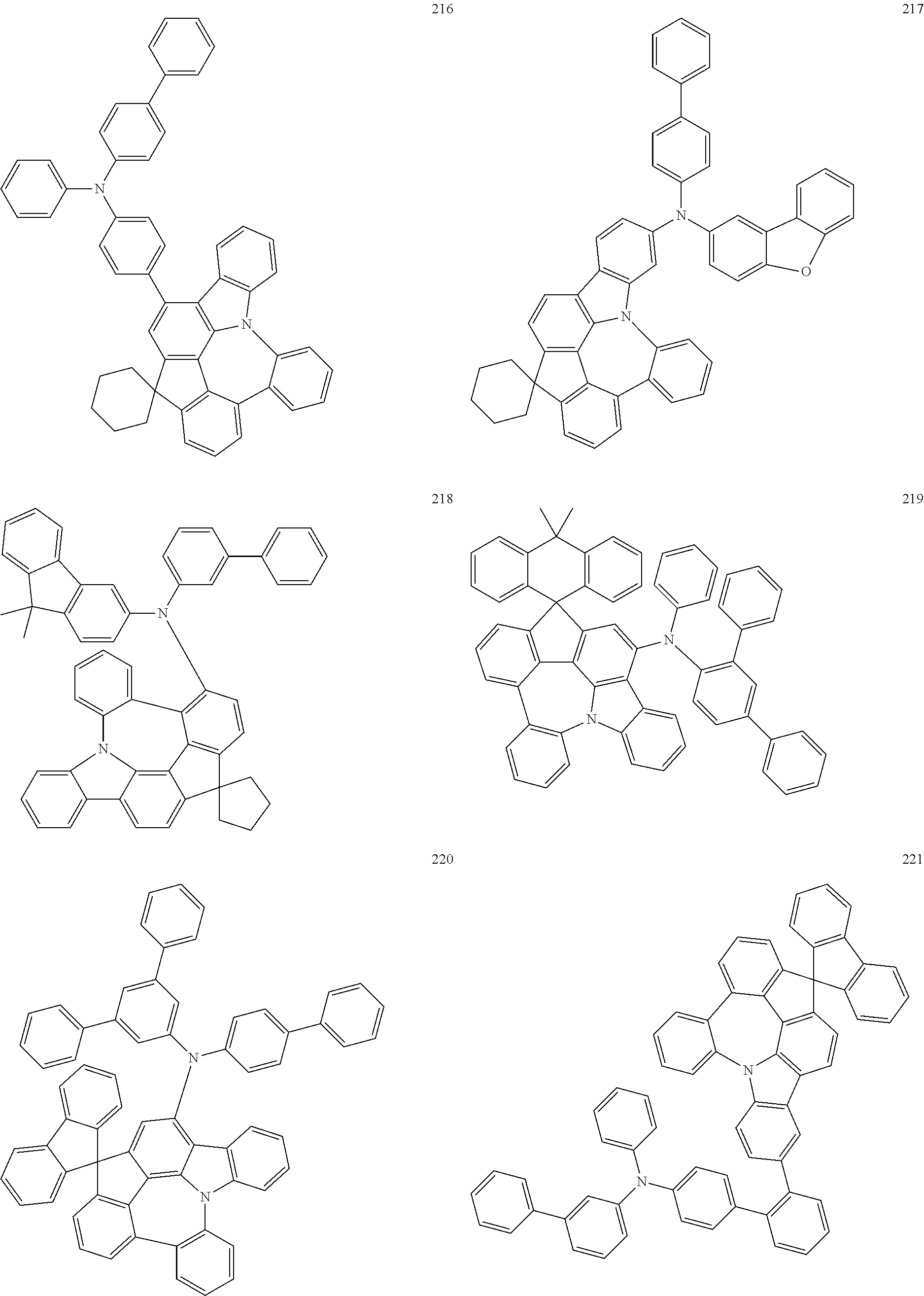

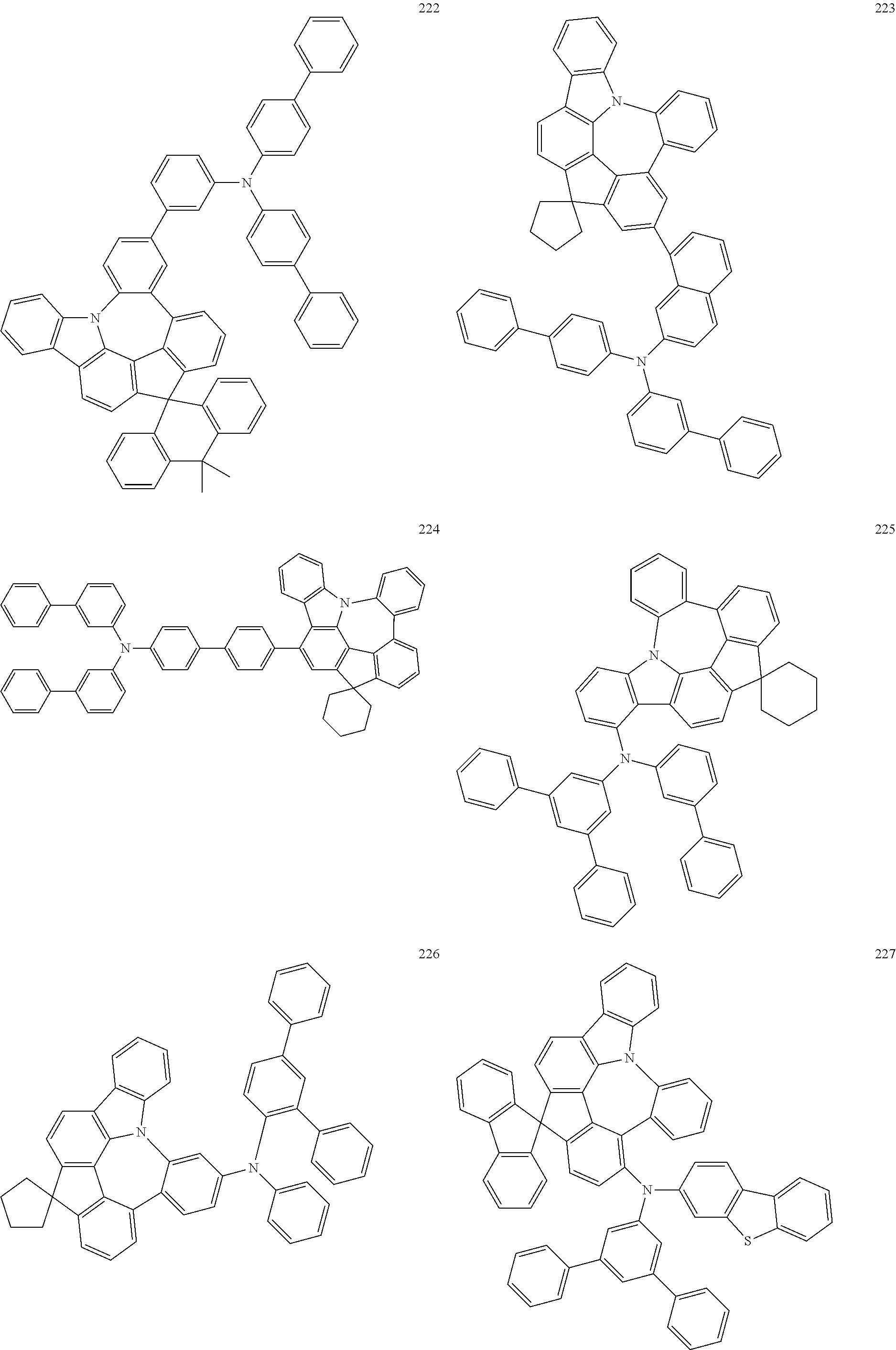

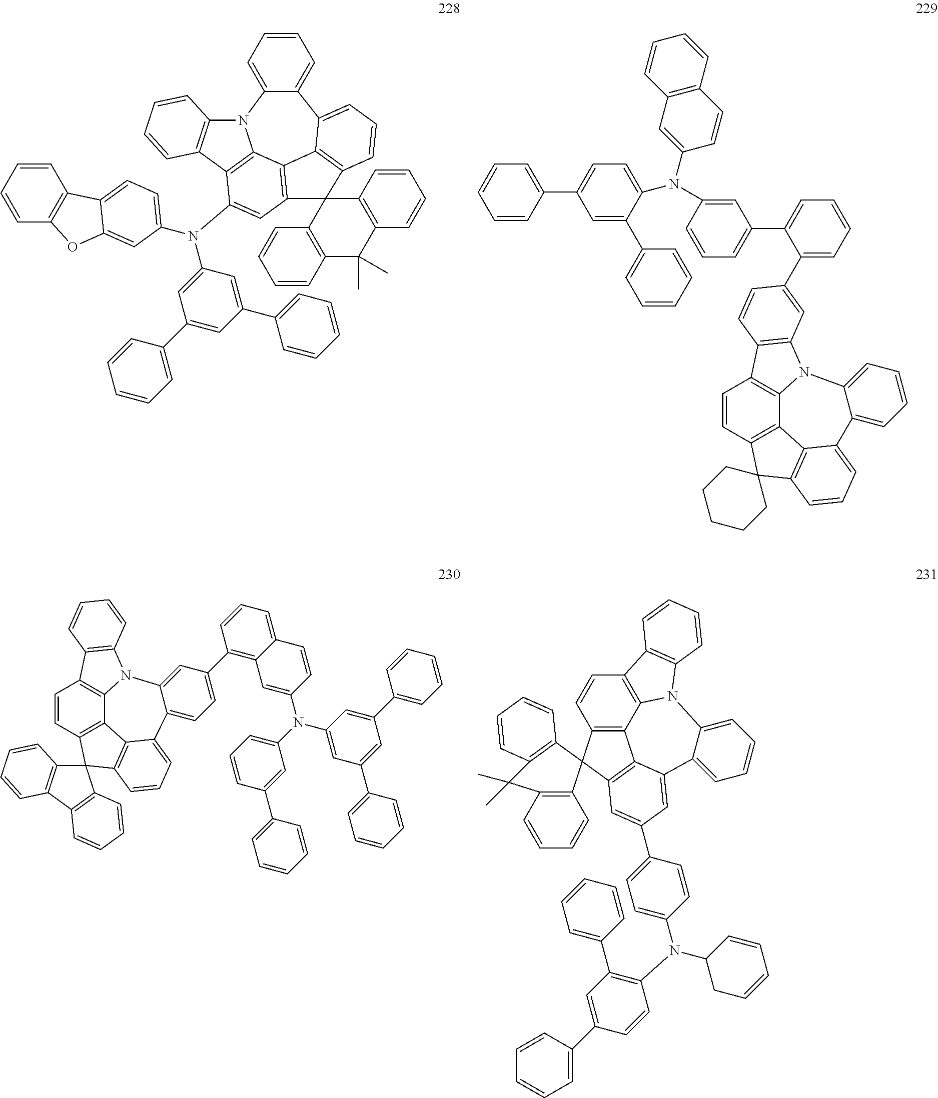

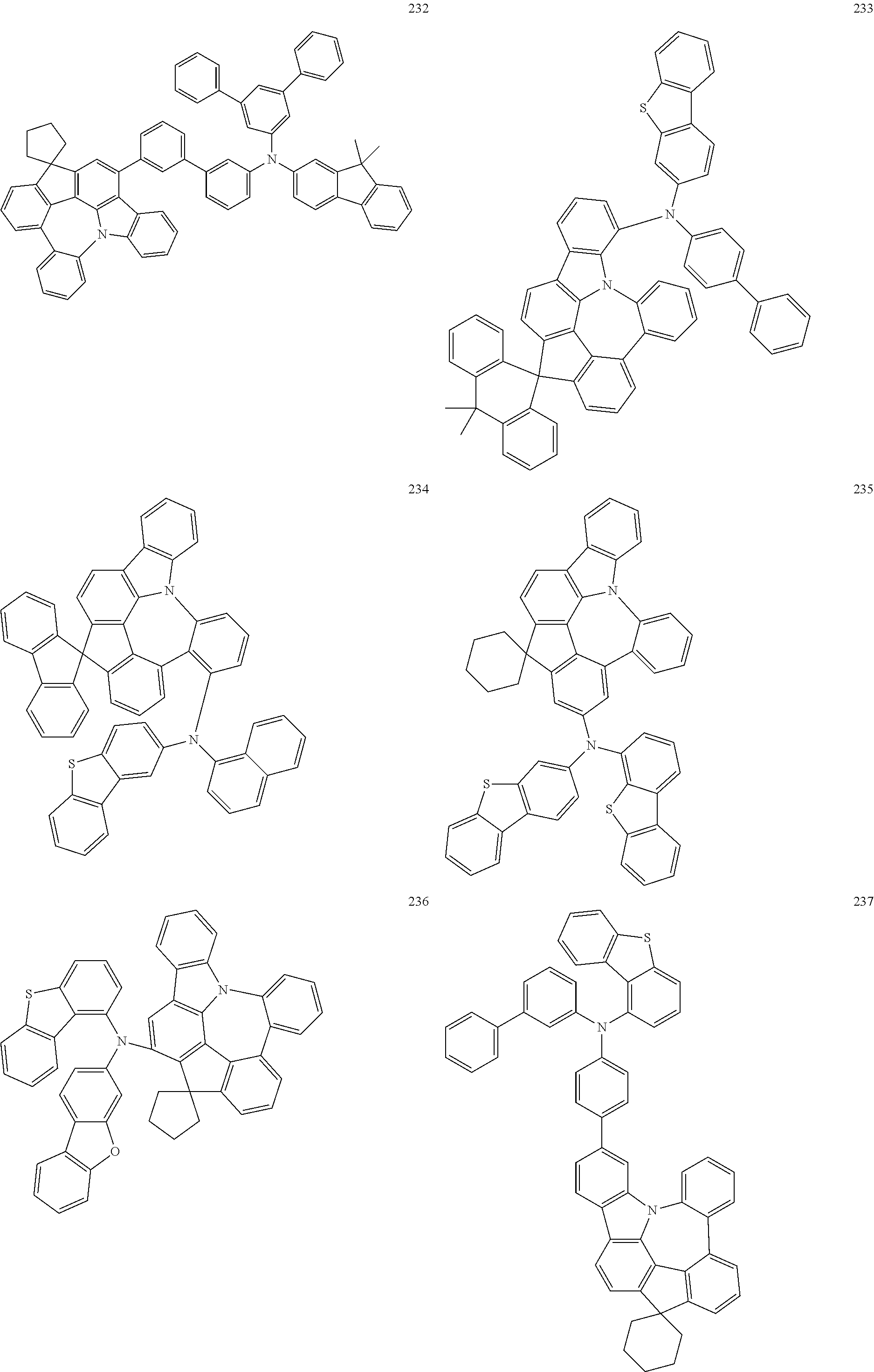

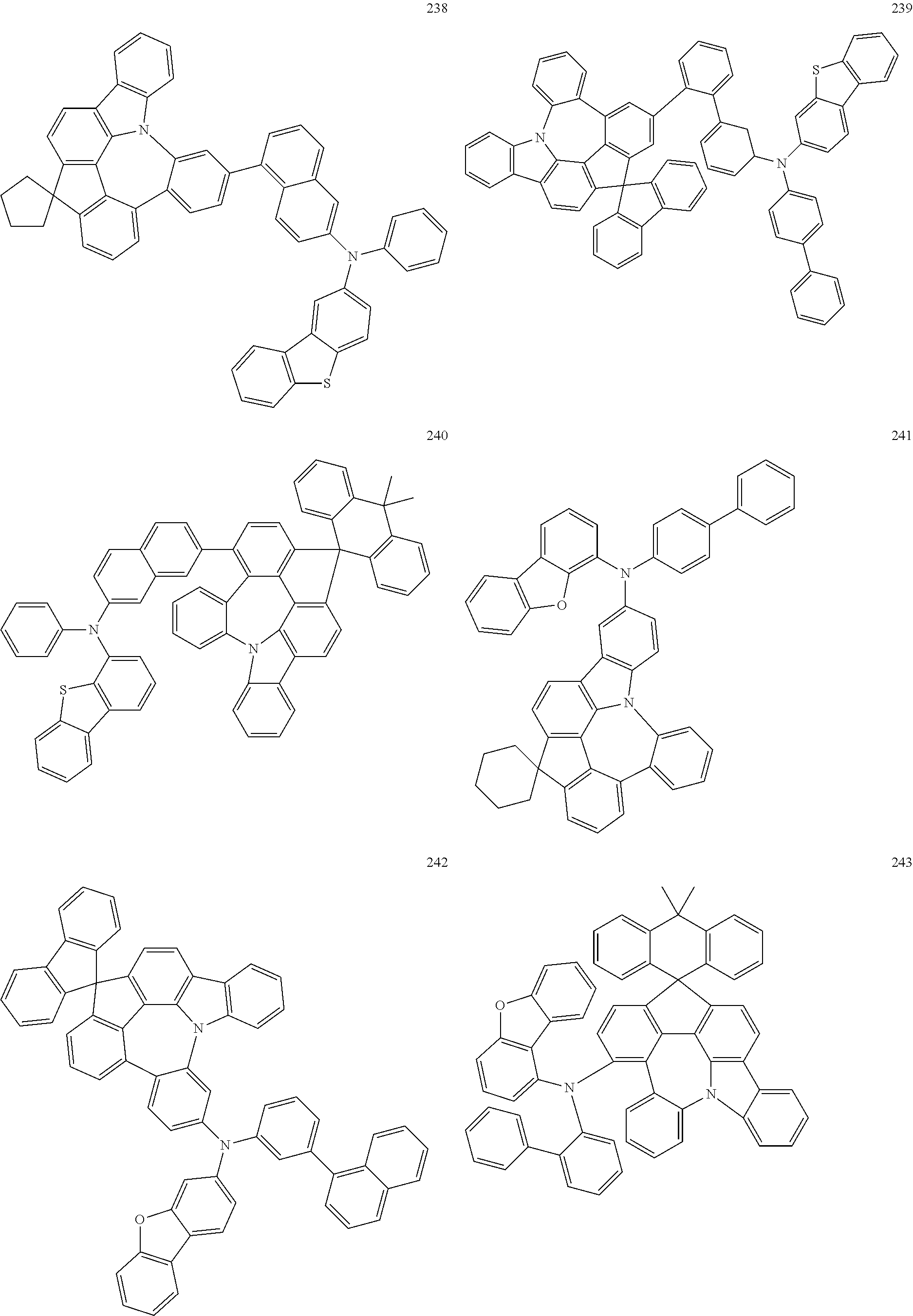

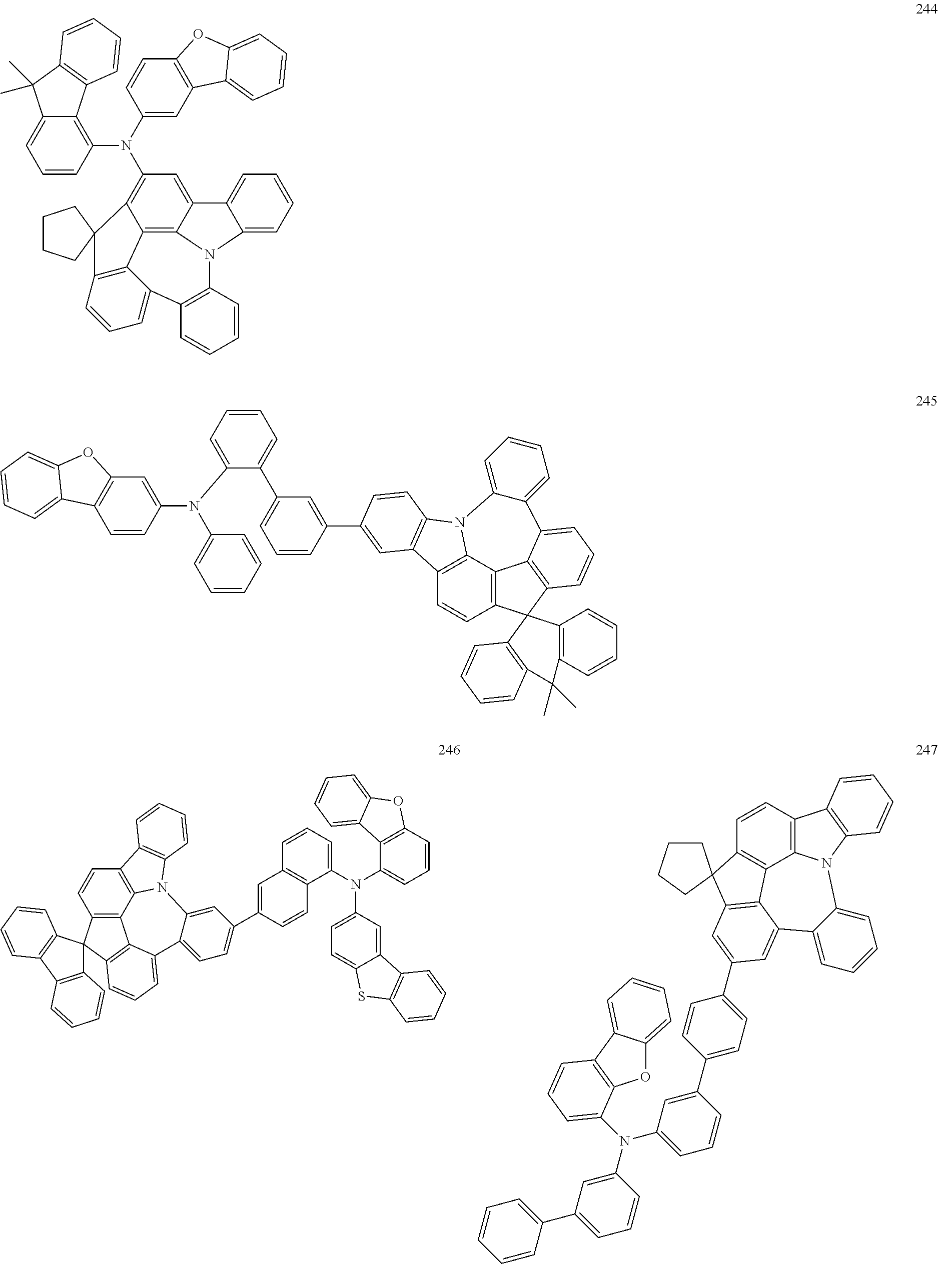 [SMILES: CC1(C)c2ccccc2-c2c(N(c3ccc4oc5ccccc5c4c3)c3cc4c5ccccc5n5c4c4c3C3(CCCC3)c3cccc(c3-4)-c3ccccc3-5)cccc21 ; CC1(C)c2ccccc2C2(c3ccccc31)c1cccc3c1-c1c2ccc2c4cc(-c5cccc(-c6ccccc6N(c6ccccc6)c6ccc7c(c6)oc6ccccc67)c5)ccc4n(c12)-c1ccccc1-3 ; c1ccc2c(c1)-c1ccccc1C21c2cccc3c2-c2c1ccc1c4ccccc4n(c21)-c1cc(-c2ccc4c(N(c5ccc6sc7ccccc7c6c5)c5cccc6oc7ccccc7c56)cccc4c2)ccc1-3 ; c1ccc(-c2cccc(N(c3cccc(-c4ccc(-c5cc6c7c(c5)C5(CCCC5)c5ccc8c9ccccc9n(c8c5-7)-c5ccccc5-6)cc4)c3)c3cccc4c3oc3ccccc34)c2)cc1]

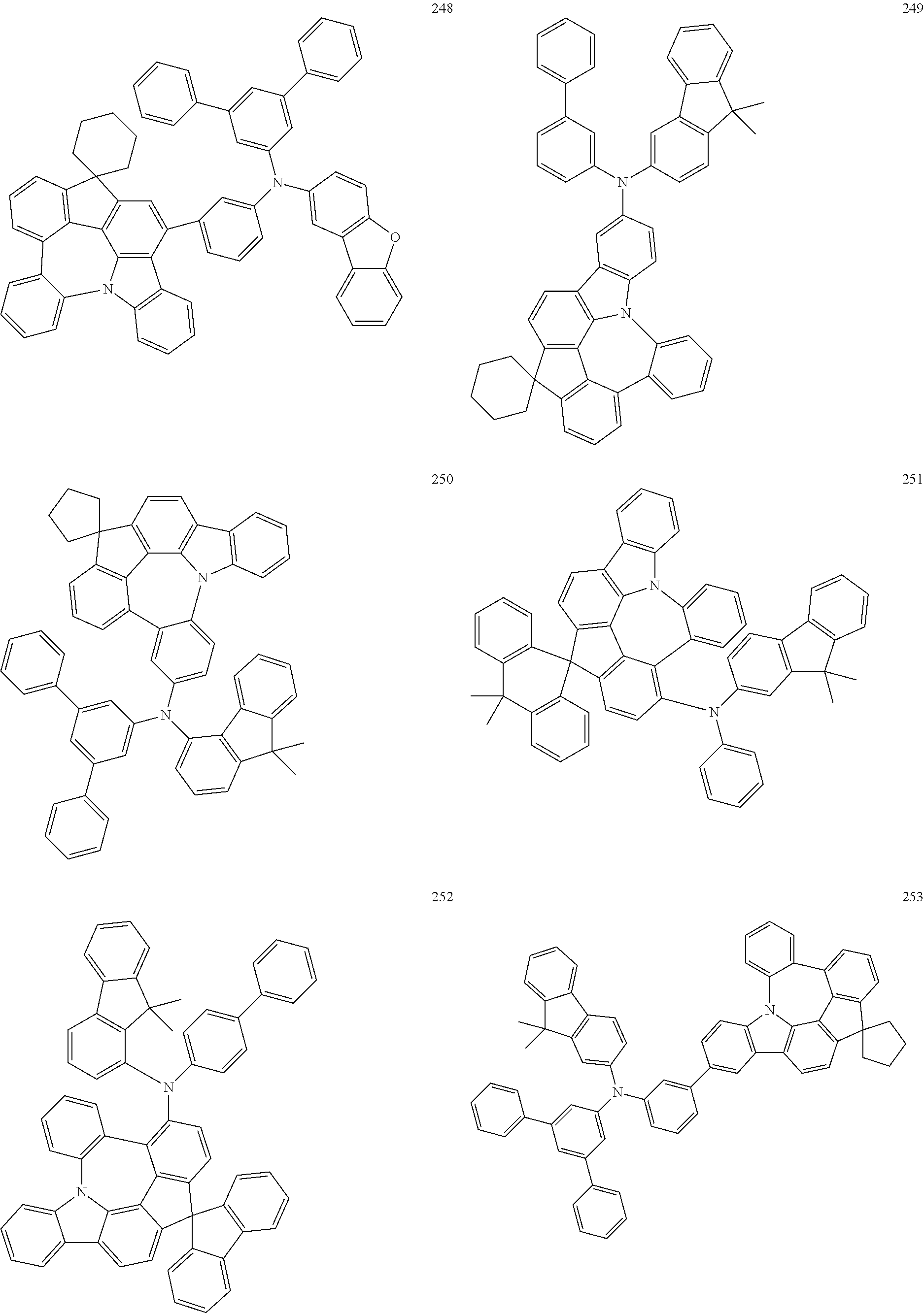

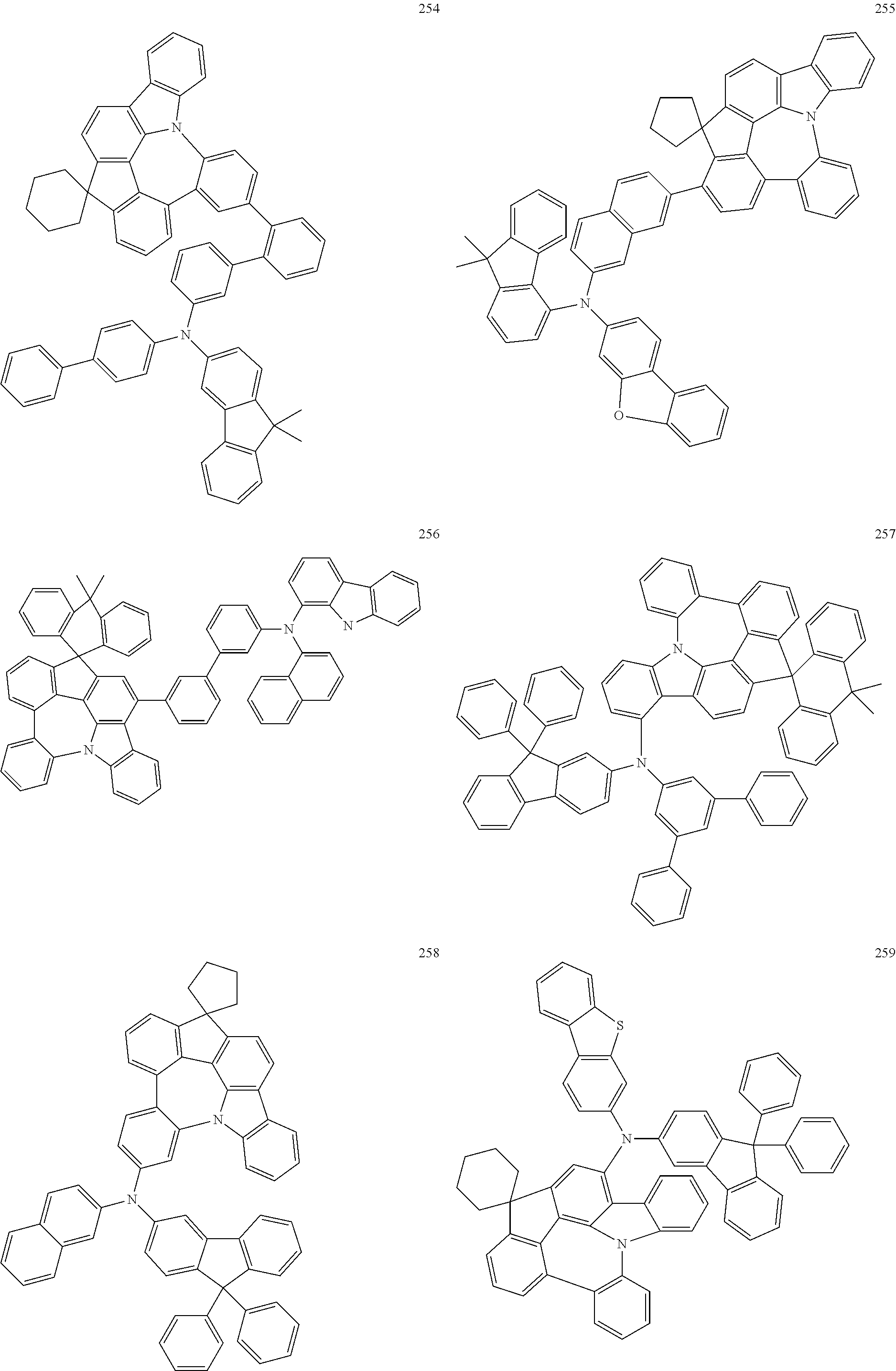

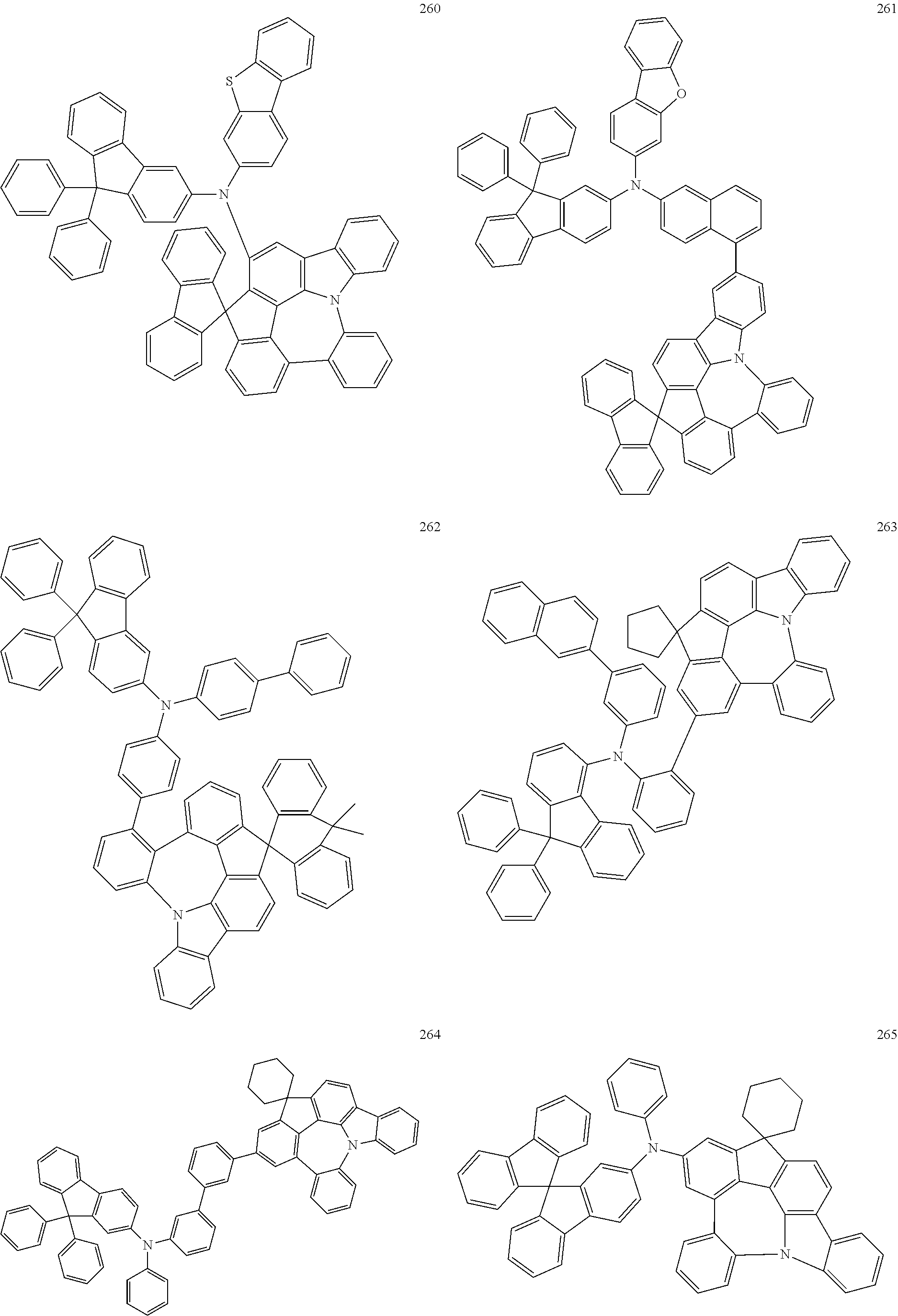

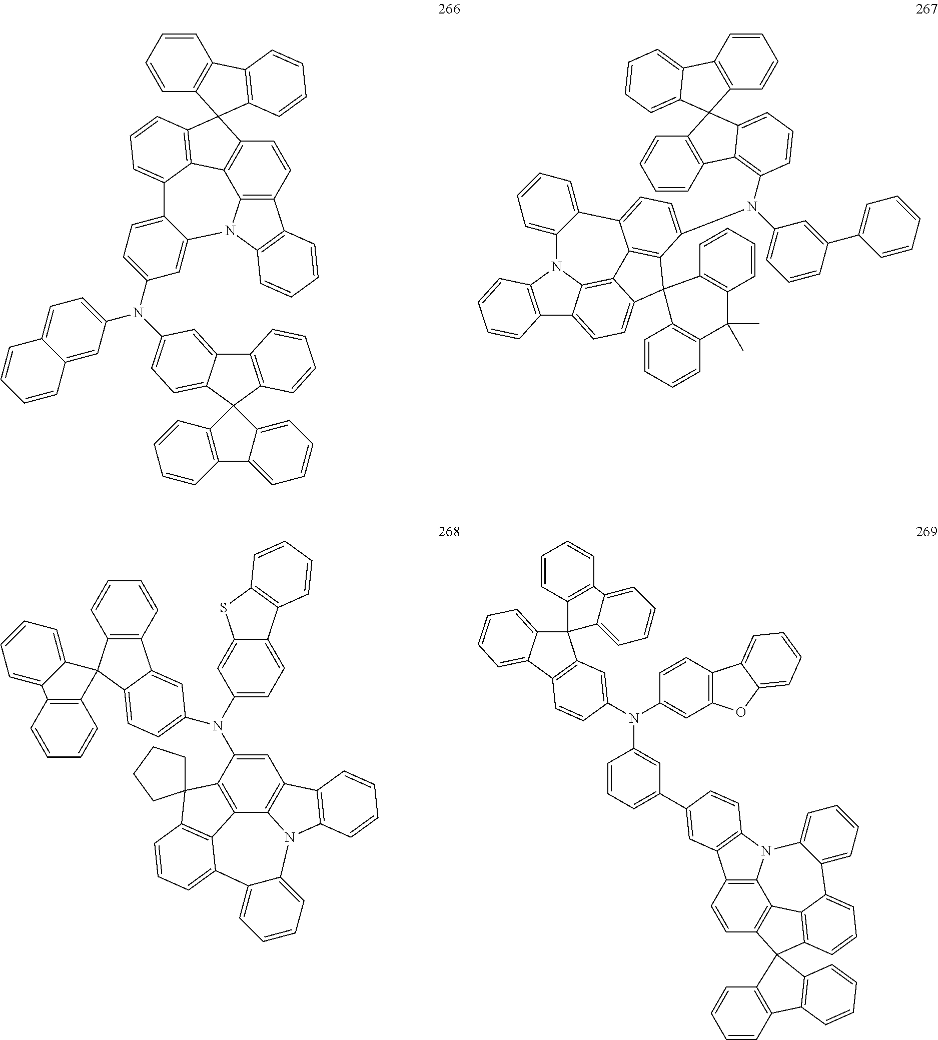

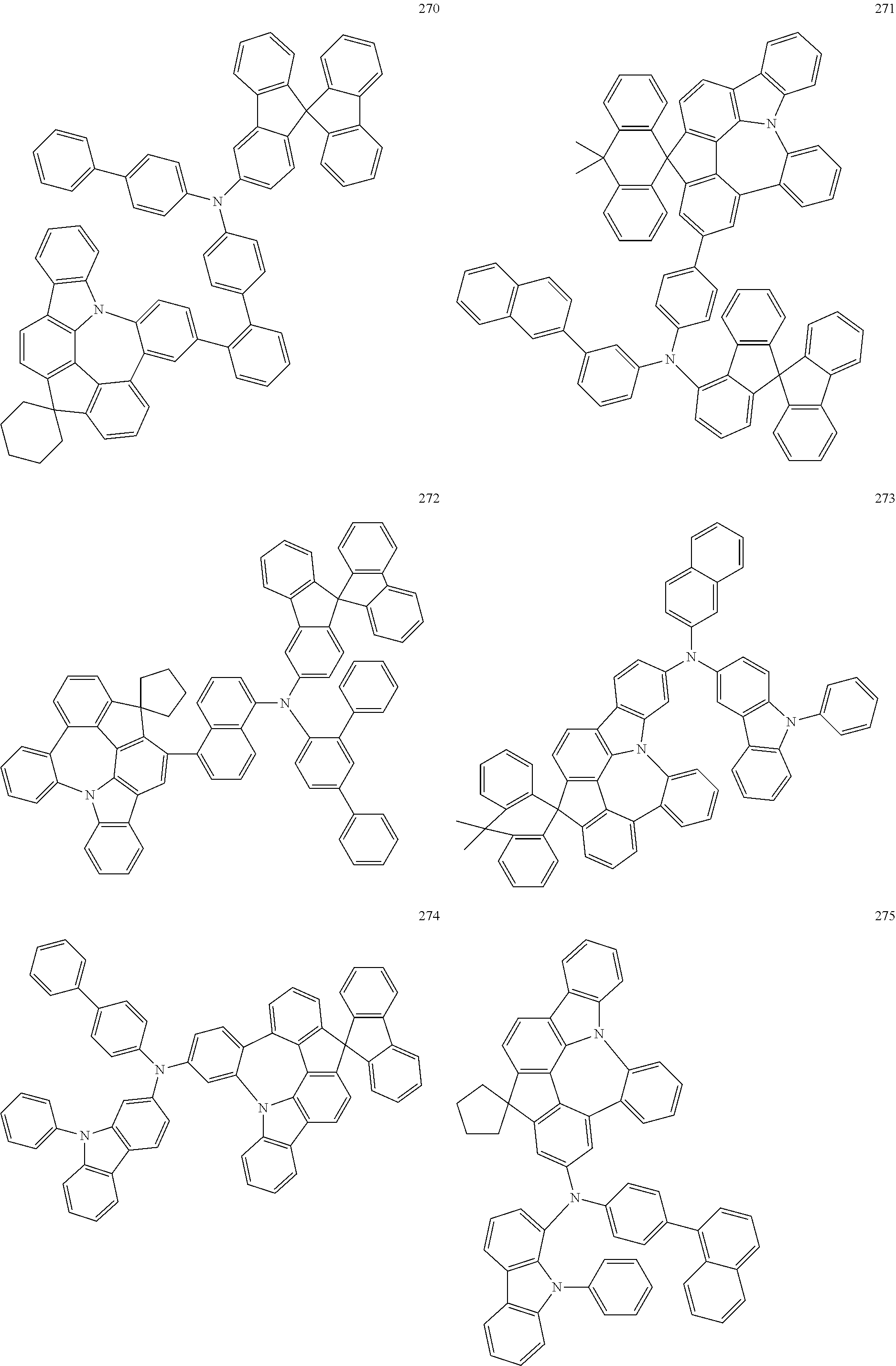

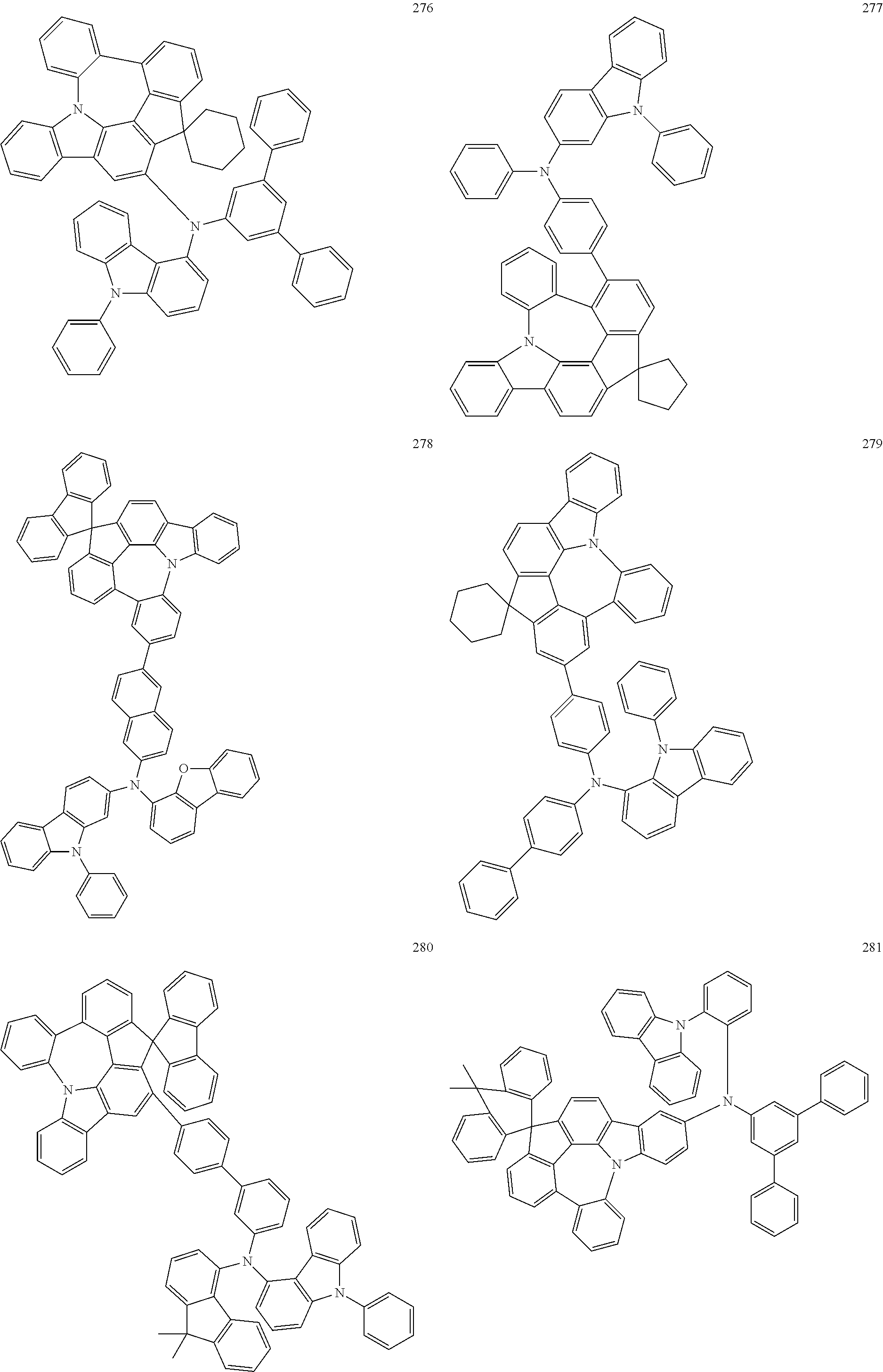

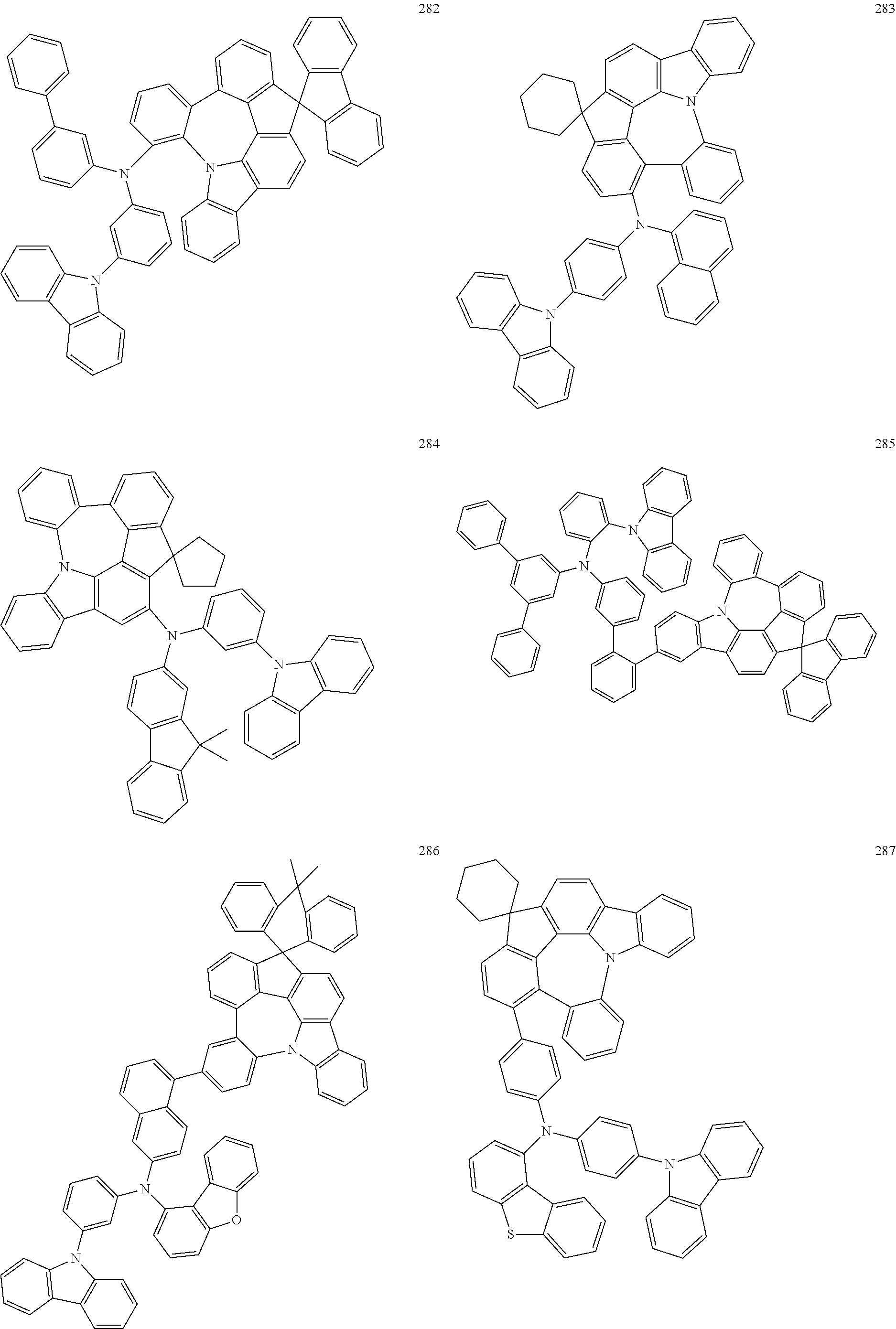

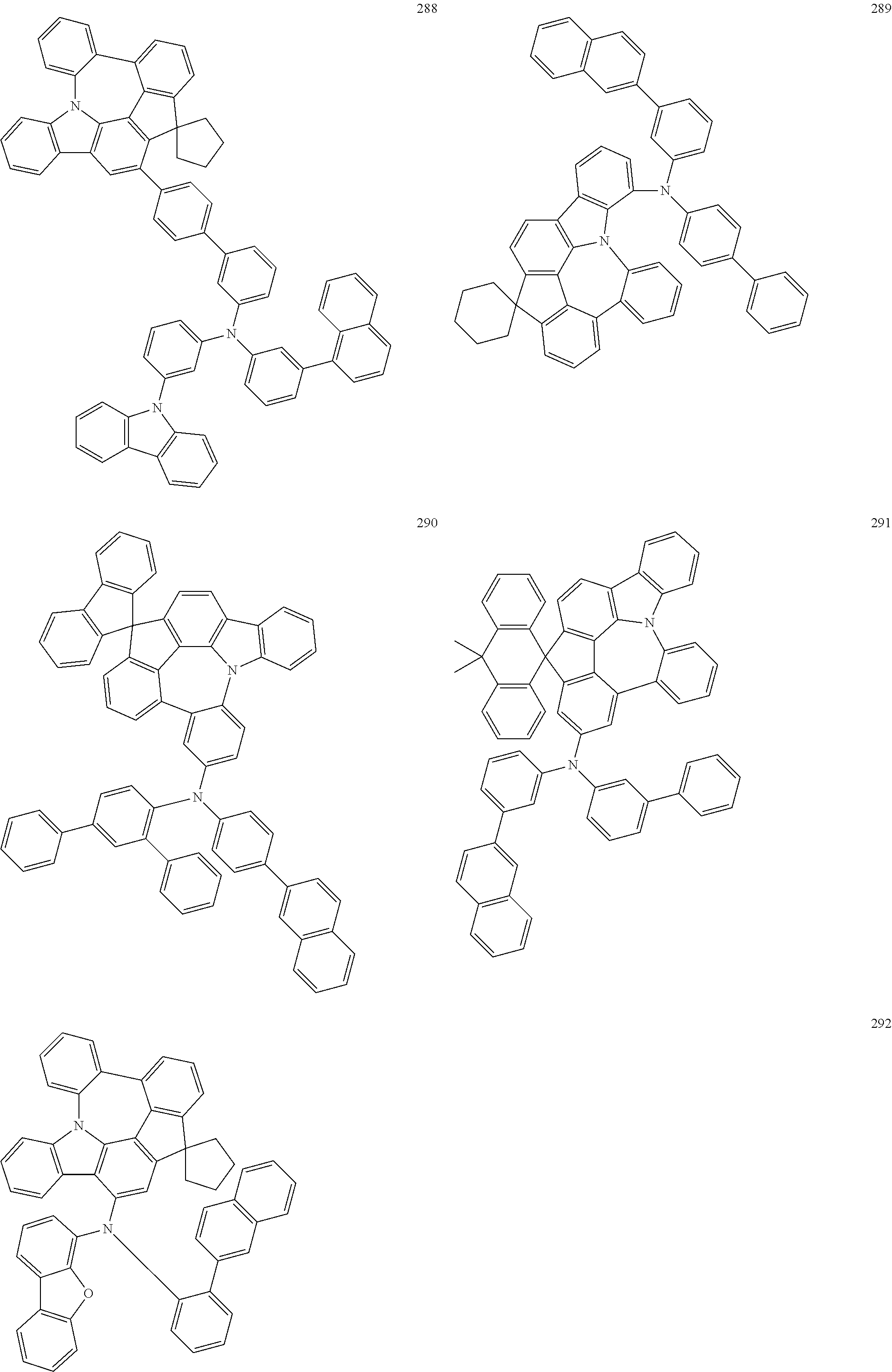

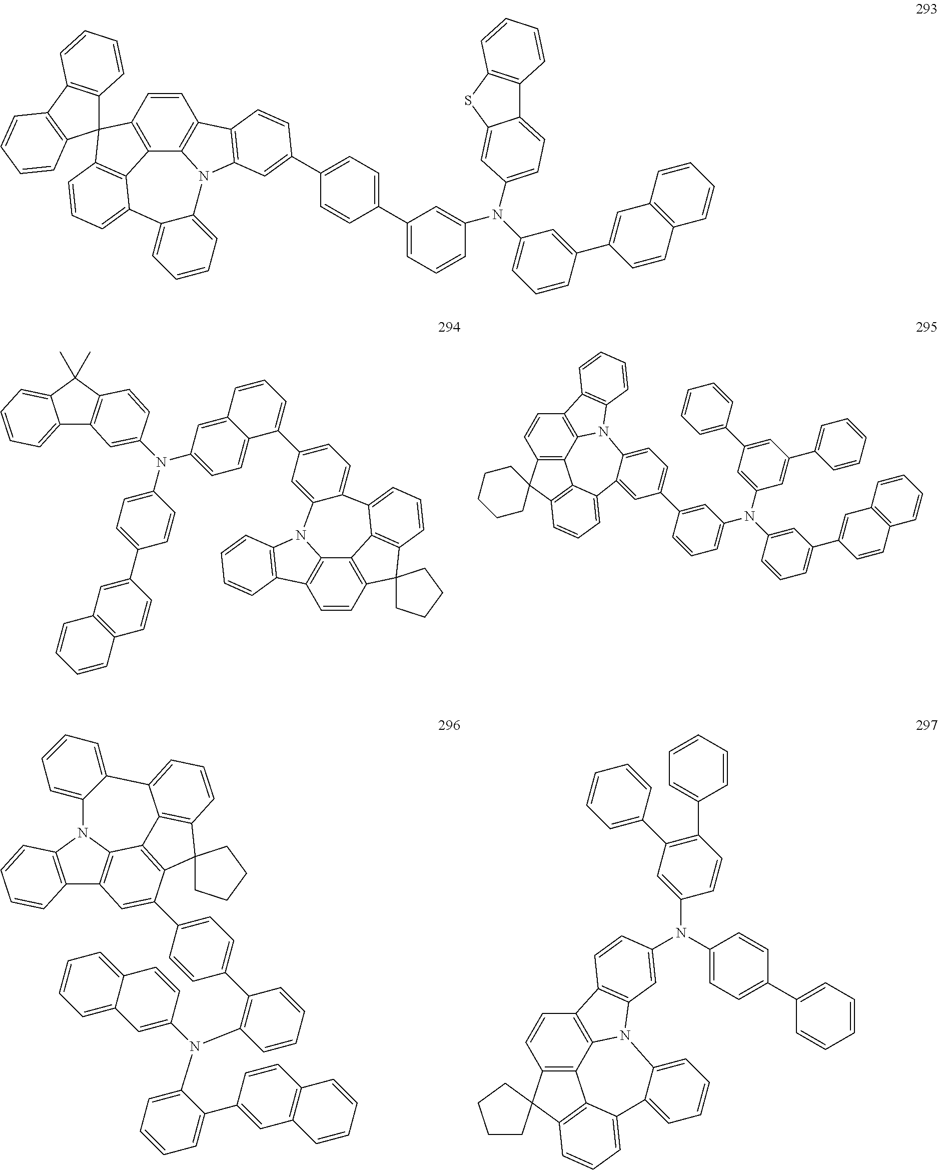

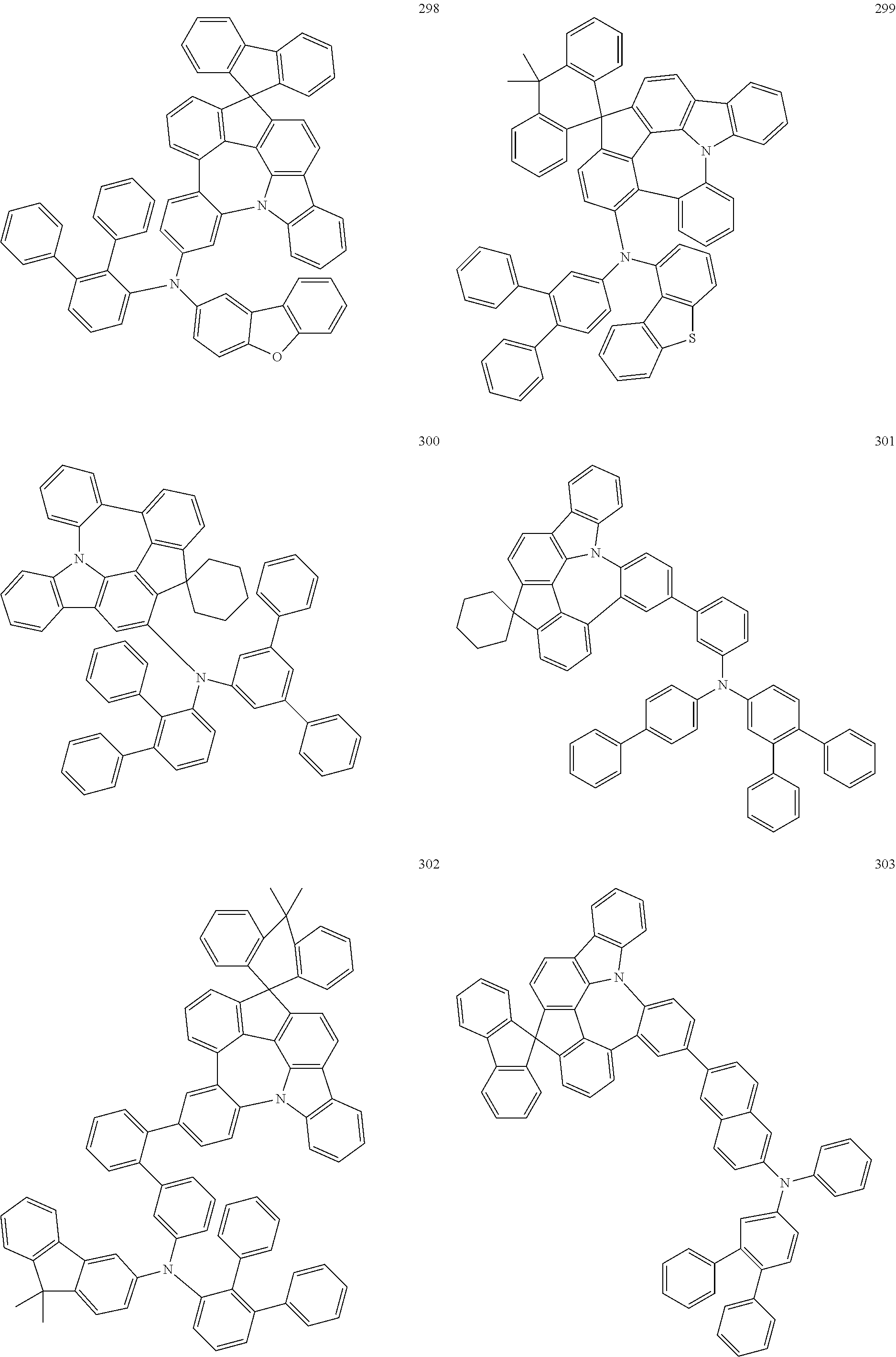

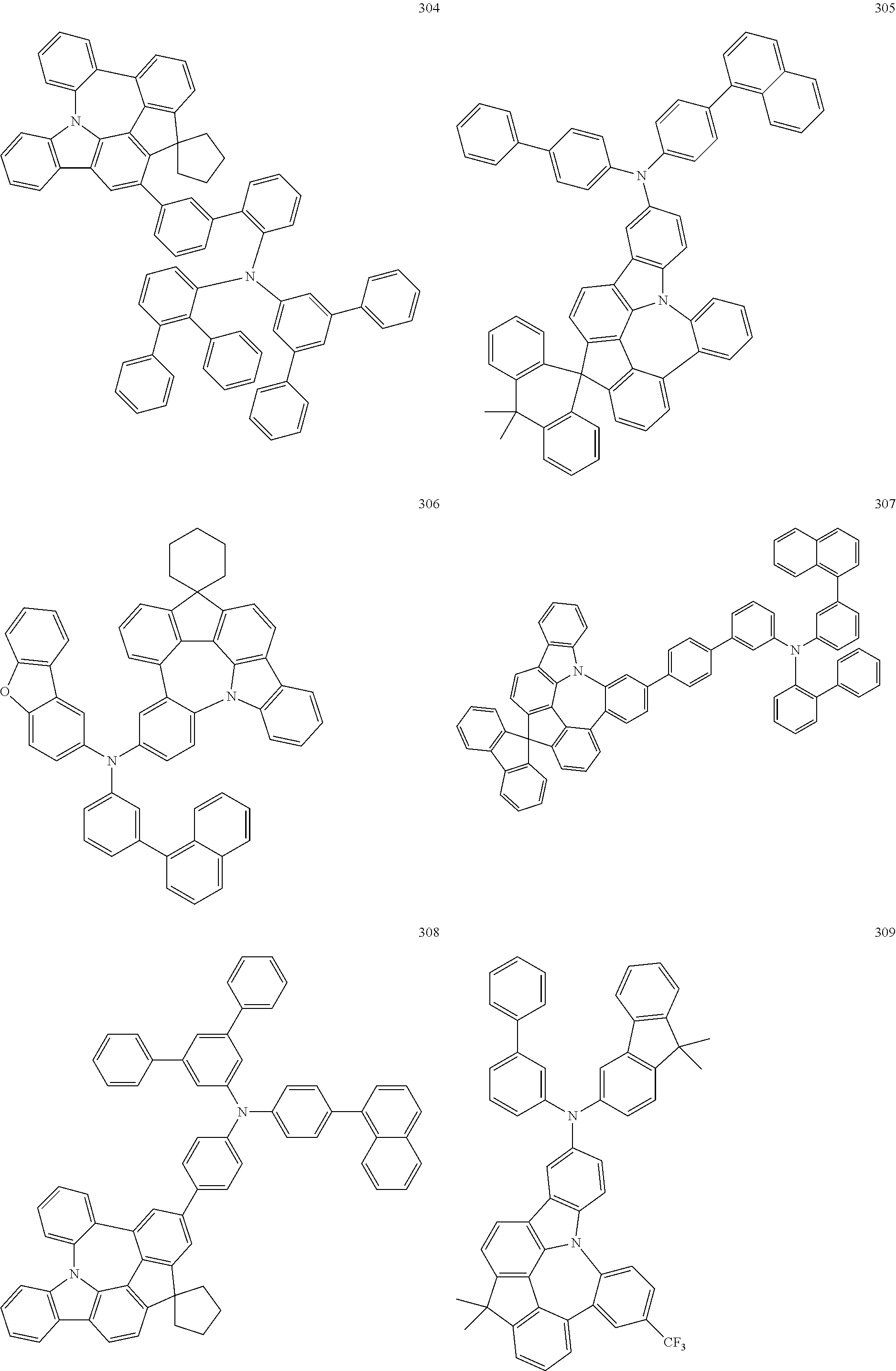

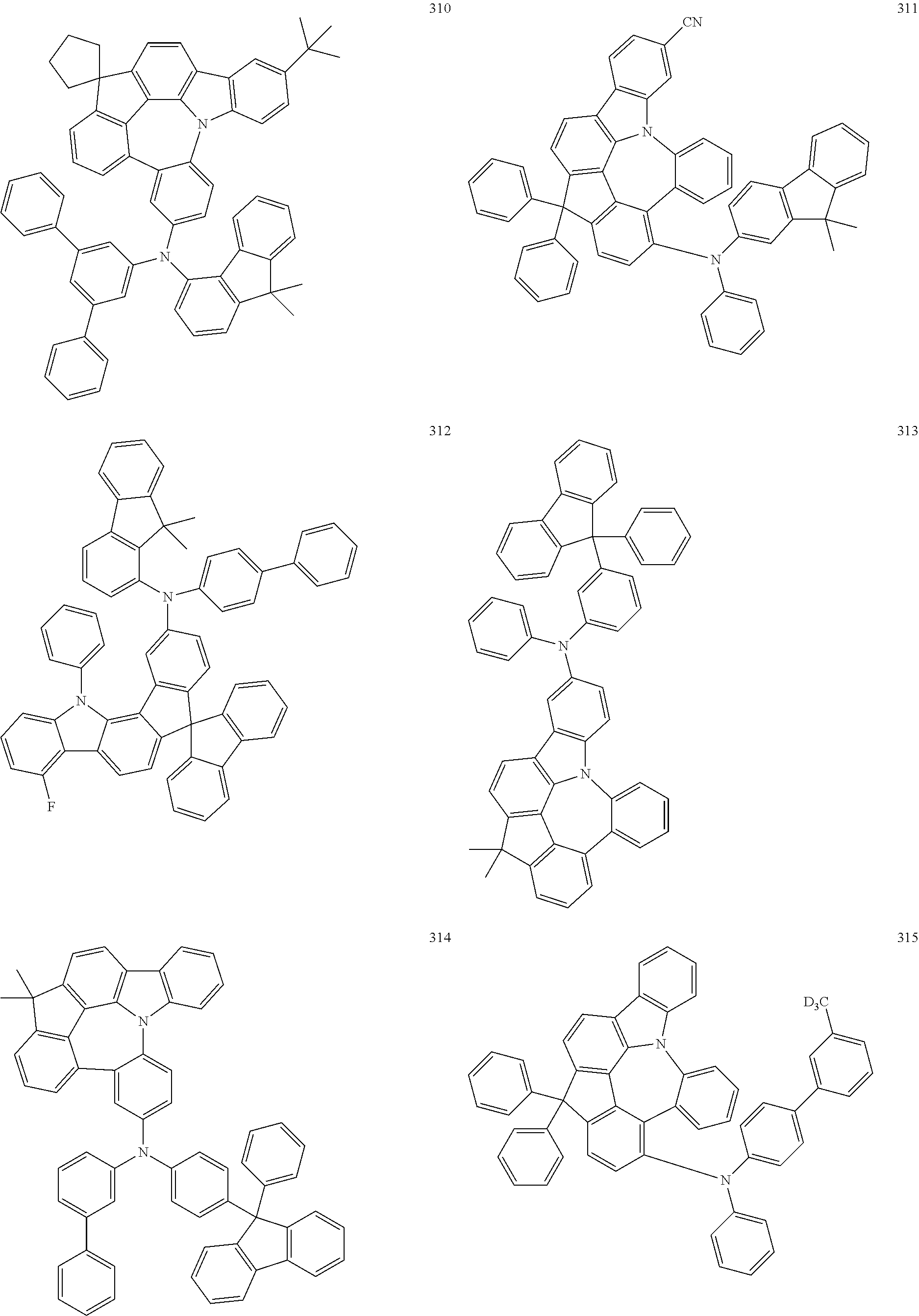

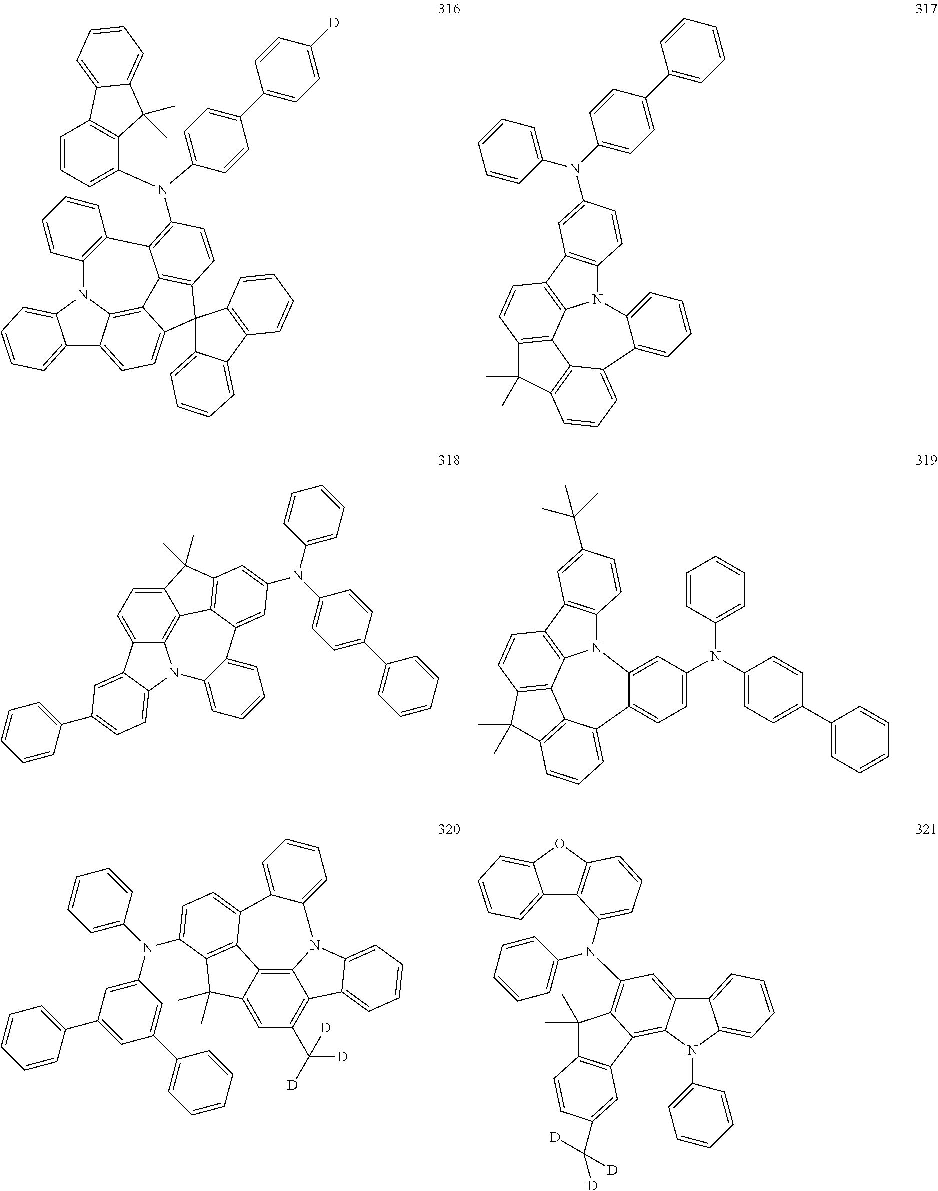

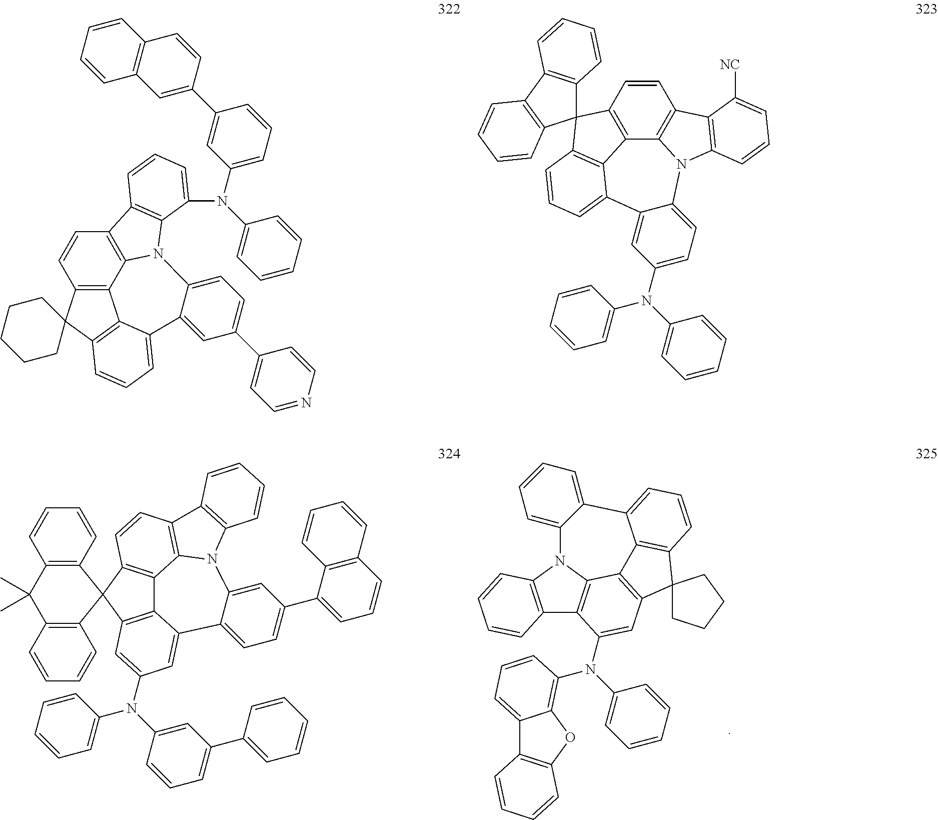 [SMILES: c1ccc(N(c2cccc(-c3ccc4ccccc4c3)c2)c2cccc3c4ccc5c6c4n(c23)-c2ccc(-c3ccncc3)cc2-c2cccc(c2-6)C52CCCCC2)cc1 ; N#Cc1cccc2c1c1ccc3c4c1n2-c1ccc(N(c2ccccc2)c2ccccc2)cc1-c1cccc(c1-4)C31c2ccccc2-c2ccccc21 ; CC1(C)c2ccccc2C2(c3ccccc31)c1cc(N(c3ccccc3)c3cccc(-c4ccccc4)c3)cc3c1-c1c2ccc2c4ccccc4n(c12)-c1cc(-c2cccc4ccccc24)ccc1-3 ; c1ccc(N(c2cccc3c2oc2ccccc23)c2cc3c4c5c2c2ccccc2n5-c2ccccc2-c2cccc(c2-4)C32CCCC2)cc1]

14. An electronic element comprising an anode, a cathode, and at least one functional layer between the anode and the cathode, wherein the functional layer comprises the organic compound according to claim 1; and
optionally, the functional layer comprises an electron blocking layer (EBL) and/or a hole transport layer (HTL), and the EBL or the HTL comprises the organic compound.

15. The electronic element according to claim 14, wherein the electronic element is an organic light-emitting element (OLED) or a photoelectric conversion element; and
optionally, the OLED is a red light-emitting OLED or a green light-emitting OLED.

16. An electronic device having the electronic element according to claim 14.
